SEQUENCE LISTING (3 sequences)

SEQ ID NO: 1 - protein, 17 aa, Artificial Sequence
  peptide portion of compound, as shown in formula (7a) in the application as filed
  MOD_RES (1)..(1): Other groups or atoms, Z1 and Y1, as described on p. XY-XY of the application as filed
  VARIANT (2)..(2): Represents a single covalent bond, or a trifunctional amino acid such as diamino-carboxylic acid
  VARIANT (3)..(3): Represents an amino acid, a dicarboxylic acid, or a peptide moiety represented by formula (8a) on p. XY of the application as filed
  DISULFID (4)..(14)
  VARIANT (5)..(5): Represents represents an amino acid as described on p. XY of the application as filed
  VARIANT (6)..(10): Each independently represent an amino acid
  VARIANT (15)..(15): Represents an amino acid, or a peptide moiety represented by formula (8b) on p. XY of the application as filed
  VARIANT (16)..(16): Represents a single covalent bond, or a trifunctional amino acid such as diamino-carboxylic acid
  MOD_RES (17)..(17): Other groups or atoms, Z2 and Y2, as described on p. XY-XY of the application as filed
XXXCXXXXXXLVWCXXX

SEQ ID NO: 2 - protein, 13 aa, Artificial Sequence
  Fc-III-FAM, as shown on p.XYZ of the application as filed
  DISULFID (2)..(12)
  MOD_RES (13)..(13): FAM label, as shown on p. XYZ of the application as filed
DCAWHLGELVWCT

SEQ ID NO: 3 - protein, 17 aa, Artificial Sequence
  reference vector, as shown on page XYZ of the application as filed
  MOD_RES (1)..(1): ACETYLATION
  VARIANT (8)..(8): Represents lysine or ornithine
  MOD_RES (17)..(17): AMIDATION
RGNCAYHXGQIIWCTYH

(19) United States
(12) Patent Application Publication
(10) Pub. No.: US 2023/0381327 A1
(43) Pub. Date: Nov. 30, 2023

(54) REACTIVE CONJUGATES

(71) Applicant: DEBIOPHARM RESEARCH & MANUFACTURING S.A., Martigny (CH)

(72) Inventors: Léo MARX, Martigny (CH); Viktoriia POSTUPALENKO, Martigny (CH); Origéne Franz NYANGUILE, Grimisuat (CH); Jean-Manuel SEGURA, Vevey (CH); Mathilde Lucile Colette PANTIN, Thonon-les-bains (FR); Patrick GARROUSTE, Saxon (CH); Frederic LEVY, Lausanne (CH)

(21) Appl. No.: 18/248,674

(22) PCT Filed: Oct. 12, 2021
(86) PCT No.: PCT/EP2021/078181

§ 371 Date: Apr. 11, 2023

(30) Foreign Application Priority Data
Oct. 12, 2020 (WO) PCT/EP2020/078571

(51) Int. Cl. A61K 47/64; A61K 47/60; A61K 49/10; A61K 49/14; A61K 49/00; A61K 47/68

(52) U.S. Cl. CPC A61K 47/64; A61K 47/60; A61K 47/6415; A61K 49/106; A61K 49/14; A61K 49/0043; A61K 49/0056; A61K 47/6855; A61K 47/6803

(57) ABSTRACT

The present invention relates to compounds (reactive conjugates) for the chemical modification of therapeutic antibodies or proteins. The compounds enable the regioselective attachment of payloads to an antibody or antibody fragment in one single step, thereby producing a modified antibody or modified antibody fragment, which can be used for diagnosing, monitoring, imaging or treating disease.

### FIELD OF THE INVENTION

The present invention relates to compounds (hereinafter sometimes referred to as “reactive conjugates”) for the chemical modification of antibodies or antibody fragments, e.g. therapeutic antibodies. The compounds enable the regioselective attachment of one or more payloads to an antibody or antibody fragment in one single step, thereby producing a modified antibody or modified antibody fragment, which can be used for diagnosing, monitoring, imaging or treating disease and/or monitoring or imaging a treatment of said disease.

### BACKGROUND OF THE INVENTION

Traditional cancer treatments, e.g. chemotherapy, can be extremely grueling (because of the severe side effects caused by their toxicity) and also extremely hit and miss, with treatments effective in one patient being completely ineffective in another. As a result, the development of new less toxic and/or more effective treatments is an ever-present need, as is the ability to monitor the effectiveness of a treatment, e.g. enabling the distinction between “responder” and “non-responder” patients.

In response to these needs, a new class of therapeutic agents referred to as “Antibody-Drug-Conjugates” (ADCs) has emerged. ADCs harness the targeting power of antibodies, e.g., monoclonal antibodies (mAbs), to deliver a payload, e.g. a cytotoxic agent or labelling agent, directly to a cancer cell. The specific targeting of cancer cells enables the therapeutic effects of the payload to be maximized, whilst toxic effects on healthy cells are minimized. Depending on the payload, ADCs can fulfill a variety of roles, e.g. diagnostic, monitoring and/or therapeutic.

ADCs can be prepared by various methods. However, the majority of these methods lead to heterogeneous mixtures of chemically distinct ADCs having varying conjugation sites and/or payload (drug) antibody ratios (or “DAR”, or “drug load(ing)”; referring to the average number of drug molecule(s) attached to one antibody moiety). This heterogeneity can complicate manufacturing resulting in high batch-to-batch variability and sometimes unpredictable safety and efficacy. Furthermore, the achievement of ADCs having a broad variety of payloads and/or high DAR value is often limited due to payload hydrophobicity and increased tendency for aggregation of the resulting conjugates.

Consequently, methods that can result in the preparation of homogeneous mixtures, e.g. regioselective or site-specific conjugation methods, and/or conjugates having a broad range of payloads are of growing interest. Such methods can drastically increase the predictability of the DAR, and the payload (drug) conjugation site, and can serve to simplify the development and manufacturing of more defined ADCs products having more predictable safety and/or improved efficacy, e.g. a homogenous high DAR distribution. Site specific conjugations methods can also ensure antibody epitopes are not altered during the conjugation process, resulting in ADCs with retained high binding affinity to the target.

Several approaches have been developed for the regiospecific and site-specific conjugation of payloads to antibodies. However, often known approaches require the modification/engineering of the antibody, for instance through the incorporation of non-natural amino acids or through the modification of carbohydrate moieties. Such modifications may negatively affect the therapeutic efficacy and/or safety of a corresponding ADC, e.g. because of undesirable effects with regard to activity, targeting, metabolism, and/or excretion of the antibody, as well as the immune response to the antibody. Other approaches involve multiple steps, e.g. the approach set out in WO 2018/199337. Such multi-step approaches may be costly and/or laborious, making them less attractive or even unsuitable for applications where a quick and simple antibody modification process is desirable, e.g. for “point-of-care” (POC) diagnostic applications.

Accordingly, there is still a need to find alternative ways for the region- or site-specific conjugation of payloads to antibodies or antibody fragments. In particular, there is a need to find ways that do not require the engineering of the antibody or antibody fragment therebefore, and that enable the preparation of antibody drug conjugates in as few steps as possible, preferably in one single step, for example, for point of care diagniostic applications, there is a need for a simple procedure that allows site specific conjugation of payloads, e.g. signaling units such as metal chelators or fluorescent probes, to therapeutic antibodies. In particular, there is a need for a simple conjugation method that preserves the antibody binding affinity and minimizes the alteration of the distribution of the antibody in vivo. Furthermore, there is a need for ways of preparing ADCs having a broad variety of payloads and/or high drug antibody ratios.

In view of the foregoing, it is an object of the present invention to provide compounds (reactive conjugates), enabling the regioselective conjugation of payloads to an antibody or antibody fragment in one single step, without the need to engineer and/or modify the antibody or antibody fragment therebefore. It is a further object to provide kits comprising such compounds.

It is yet another object of the present invention to provide a method for producing a modified antibody or modified antibody fragment, e.g. ADCs, which can be used in methods of diagnosing, monitoring, imaging or treating disease.

### SUMMARY OF THE PRESENT INVENTION

The present invention provides a compound, which enables the regioselective attachment of one or more payloads to an antibody, e.g. to a therapeutic antibody, or to an antibody fragment that is optionally incorporated into an Fc-fusion protein. This regioselective attachment can be accomplished in one single (conjugation) step. The resulting modified antibody or modified antibody fragment, e.g. ADC or antibody-radionuclide conjugate, can be used in a method of diagnosing, monitoring, imaging or treating disease, in particular cancer.

The compound (reactive conjugate) of the present invention is represented by the following formula (1):

V—(Y—P)ₙ (1)
wherein,
V is a peptide comprising a vector that is capable of interacting with the fragment crystallizable (Fc) region of an antibody or fragment thereof, said antibody fragment optionally being incorporated into an Fc-fusion protein;
P is a group comprising one or more payloads P¹;
Y is a reactive moiety capable of reacting with the side chain of an amino acid, preferably lysine, wherein Y is covalently attached to the side chain of an amino acid comprised in V; and
n is an integer from 1 to 3, preferably 1 or 2, and most preferably 1.

The present invention also relates to a kit for the regioselective modification of an antibody or antibody fragment, the antibody fragment being optionally incorporated into an Fc-fusion protein, wherein said kit comprises the compound as described hereinbefore, optionally immobilized on a solid phase matrix, e.g. on beads, and a buffer.

Furthermore, the present invention relates to a method for the regioselective modification of an antibody or antibody fragment, the antibody fragment being optionally incorporated into an Fc-fusion protein, wherein said method employs the compound described hereinbefore.

Additionally, the present invention relates to a modified antibody or modified antibody fragment (e.g. obtainable or obtained by the method described hereinbefore), the antibody fragment being optionally incorporated into an Fc-fusion protein, for use in a method of diagnosing, monitoring, imaging and/or treating disease, especially cancer.

The present invention, in particular, includes the following embodiments (“Items”):
1. A compound of formula (1):

V—(Y—P)ₙ (1)
wherein,
V is a peptide comprising a vector that is capable of interacting with the fragment crystallizable (Fc) region of an antibody or fragment thereof, said antibody fragment optionally being incorporated into an Fc-fusion protein;
P is a group comprising one or more payloads P¹;
Y is a reactive moiety capable of reacting with the side chain of an amino acid, preferably lysine, wherein Y is covalently attached to the side chain of an amino acid comprised in V; and
n is an integer from 1 to 3, preferably 1 or 2, and most preferably 1.
2. The compound of item 1, wherein P is P¹, or P is represented by one of the following formulae (2a), (2b) and (2c):

P¹-L-* (2a)

(P¹-L)ₙ′-K—* (2b)

(P¹)ₙ′-K—* (2c)
wherein,
P¹ is a payload;
L is a linker, wherein said linker is optionally cleavable, and wherein preferably the linker comprises one or more atoms selected from carbon, nitrogen, oxygen, phosphorus and sulfur;
K is a branching group that is covalently bonded to group Y and to two or more linkers (L) in formula (2b), or two or more payloads (P¹) in formula (2c), so as to form a dendrimeric structure;
n′ is an integer of from 2 to 8, preferably from 2 to 4, and more preferably 2; and
* indicates covalent attachment to the reactive moiety (Y).
3. The compound of item 1 or 2, wherein P¹ is selected from:
   (i) a moiety derived from
      a chromophore, wherein said chromophore is preferably selected from
         a phosphorophore, and
         a fluorophore such as fluorescein or rhodamine,
      a labelling moiety which may include a radionuclide, wherein said labelling moiety is preferably a moiety containing, or capable of containing, a radionuclide, and more preferably selected from
         a labelling moiety containing, or capable of containing a non-metallic radionuclide such as ¹²⁵I, ¹²³I, ¹³¹I, ¹¹C, ¹⁵O, ¹⁸F, such as a moiety derived from 4-hydroxyphenylpropionate containing a radionuclide such as ¹²⁵I, ¹²³I or ¹³¹I, and
         a chelating agent optionally comprising a chelated radionuclide such as a chelating agent derived from diethylenetriamine pentaacetic acid (DTPA), cyclohexyl diethylenetriamine pentaacetic acid (CH-X-DTPA), desferrioxamine (DFO), N1-(27-amino-11,22-dihydroxy-7,10,18,21-tetraoxo-6,11,17,22-tetraazaheptacosyl)-N1-hydroxy-N4-(5-(N-hydroxyacetamido)pentyl)succinimide (DFO′), N1-(5-(3-(4-aminobutyl)-1-hydroxy-2-oxopiperidine-3-carboxamido)pentyl)-N1-hydroxy-N4-(5-(N-hydroxy-4-((5-(N-hydroxyacetamido)pentyl)amino)-4-oxobutanamido)pentyl)succinimide (DFO-cyclo′), 1-(1,3-carboxypropyl)-4,7-carboxymethyl-1,4,7-tetraacetic acid (NODAGA), 1,4,7,10-tetraazacyclododecane-1-glutaric acid-4,7,10-triacetic acid (DOTAGA), 2,2′-(1,4,7-triazacyclononane-1,4-diyl)diacetate (NO2A), 1,4,7,10-tetraatacyclododecane-1,4,7,10-tetraacetic acid (DOTA), mercaptoacetyl-glycyl-glycyl-glycine (maGGG), mercaptoacetyl-serine-serine-serine (maSSS), 1,4,7,10-tetraatacyclododecane-1,4,7,10-tetraacetic acid-methonine (DOTA-Met), 1,4,7-triazacyclononane-1,4,7-triacetic acid (NOTA), ethylenediaminetetraacetic acid (EDTA), ethylenediaminediacetic acid, triethylenetetraminehexaacetic acid (TTHA), 1,4,8,11-tetraazacyclotetradecane (CYCLAM), 1,4,8,11-tetraazacyclotetradecane-1,4,8,11-tetraacetic acid (TETA), 1,4,8,11-tetraazabicyclo[6.6.2]hexadecane-4,11-diaceticacid (CB-TE2A), 2,2′,2″-(1,4,7,10-tetraazacyclododecane-1,4,7-triyl)triacetamide (DO3AM), 1,4,7,10-tetraazacyclododecane-1,7-diacetic acid (DO2A), 1,5,9-triazacyclododecane (TACD), (3a1s,5a1s)-dodecahydro-3a,5a,8a,10a-tetraazapyrene (cis-glyoxal-cyclam), 1,4,7-triazacyclononane (TACN), 1,4,7,10-tetraazacyclododecane (cyclen), tri(hydroxypyridinone) (THP), 3-(((4,7-bis((hydroxy(hydroxymethyl)phosphoryl)methyl)-1,4,7-triazonan-1-yl)methyl)(hydroxy)phosphoryl)propanoic acid (NOPO), 3,6,9,15-Tetraazabicyclo[9.3.1]pentadeca-1(15),11,13-triene-3,6,9-triacetic acid (PCTA), 2,2′,2″,2′″-(1,4,7,10-tetraazacyclotridecane-1,4,7,10-tetrayl)tetraacetic acid (TRITA), 2,2′,2″,2′″-(1,4,7,10-tetraazacyclotridecane-1,4,7,10-tetrayl)tetraacetamide (TRITAM), 2,2′,2″-(1,4,7,10-tetraazacyclotridecane-1,4,7-triyl)triacetamide (TRITRAM), trans-N-dimethyl-cyclam, 2,2′,2″-(1,4,7-triazacyclononane-1,4,7-triyl)triacetamide (NOTAM), oxocyclam, dioxocyclam, 1,7-dioxa-4,10-diazacyclododecane, cross-bridged-cyclam (CB-cyclam), triazacyclononane phosphinate (TRAP), dipyridoxyl diphosphate (DPDP), meso-tetra-(4-sulfanotophenyl)porphine (TPPS₄), ethylenebishydroxyphenylglycine (EHPG), hexamethylenediaminetetraacetic acid, dimethylphosphinomethane (DMPE), methylenediphosphoric acid, dimercaptosuccinic acid (DMPA), 1,4,7,10-tetraaza-1,4,7,10-tetra(2-carbamoylmethyl)cyclododecane (TCMC), or derivatives thereof;
   (ii) a moiety comprising a conjugation group selected from an optionally substituted conjugated diene, an optionally substituted tetrazine (TZ), an optionally substituted alkyne or azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, an optionally substituted halogenoacetamide, an optionally substituted maleimide, and an optionally substituted or protected thiol, said thiol being preferably protected with a monomethoxytrityl group;
   (iii) a moiety derived from a drug selected from
      an antineoplastic agent such as
         a DNA-alkylating agent e.g. duocarmycin,
         a topoisomerase inhibitor e.g. doxorubicin,
         an RNA-polymerase II inhibitor e.g. alpha-amanitin,
         a DNA cleaving agent e.g. calicheamicin,
         an antimitotic agent or microtubule disruptor e.g. a taxane an auristatin or a maytansinoid,
         an anti-metabolite, such as a derivatives of gemcitabine,
         a Kinesin spindle protein inhibitor such as Filanesib,
         a kinase inhibitor such as ipatasertib or gefitinib,
         nicotinamide phosphoribosyltransferase inhibitor e.g. 2241014-82-2,
         a matrix metallopeptidase 9 inhibitor such as derivatives of CGS27023A,
         a phosphatase inhibitor such as mycrocystin-LR
      an immunomodulatory agent such as fluticasone,
      an anti-infectious disease agent such as rifamycin, clindamycin or reptamulin, and
      radioisotopes, metabolites, pharmaceutically acceptable salts, and/or prodrugs of any of the foregoing;
   (iv) a moiety comprising one or more solubilizing groups, each solubilizing group being preferably independently selected from the group consisting of moieties comprising one or more ionic groups, such as ammonium, sulfate, or sulfonate groups, and polyalkylene oxide groups; wherein the moiety preferably comprises one or more C₂₋₃ polyalkylene oxide groups, and wherein preferably each C₂₋₃ polyalkylene oxide group independently comprises from 4 to 600, preferably from 10 to 200, more preferably from 15 to 80 repeating units;
      if more than one payload (P¹) is present, each P¹ is independently selected from the aforementioned moieties (i) to (iii) or (i) to (iv), the payloads P¹ being preferably identical to each other.
4. The compound of any one of items 1 to 3, wherein P¹ is a chelating agent that optionally comprises a chelated radionuclide, said chelating agent preferably being a moiety derived from DTPA, DOTA, DFO, NOTA, PCTA, CH-X-DTPA, NODAGA, DOTAGA, maSSS, maGGG or DOTA-Methionine, and more preferably a moiety derived from DOTA, DTPA, CH-X-DTPA, PCTA, NOTA or DFO.
5. The compound of item 3 or 4, wherein the radionuclide is selected from ¹²⁴I, ¹³¹I, ⁸⁶Y, ⁹⁰Y, ¹⁷⁷Lu, ¹¹¹In, ¹⁸⁸Re, ⁵⁵G, ⁶⁴Cu, ⁶⁷Cu, ⁶⁸Ga, ⁸⁹Zr, ²⁰³Pb, ²¹²Pb, ²¹²Bi, ²¹³Bi, ⁷²As, ²¹¹At, ²²⁵Ac, ²²³Ra, ⁹⁷Ru, ¹⁴⁹Tb, ¹⁵²Tb, ¹⁶¹Tb, ⁹⁹mTc, ²²⁶Th, ²²⁷Th, ²⁰¹Tl, ⁸⁹Sr, ^{44/43}Sc, ⁴⁷Sc, ¹⁵³Sm, ¹³³Xe, and Al¹⁸F, preferably from ⁸⁹Zr, ¹¹¹In, ⁶⁴Cu, ¹⁷⁷Lu, ⁶⁸Ga, ⁹⁹mTc, ²⁰³Pb, ⁷²As, ⁵⁵Co, ⁹⁷Ru, ²⁰¹Tl, ¹⁵²Tb, ¹³³Xe, ⁸⁶Y, and Al¹⁸F, more preferably from ⁸⁹Zr, ¹¹¹In, ⁶⁴Cu, ¹⁷⁷Lu, ⁶⁸Ga, and ^{99m}Tc, and most preferably from ⁶⁴Cu, ⁹⁹mTc, and ¹¹¹In.
6. The compound of any one of items 1 to 3, wherein P¹ is a moiety derived from exatecan, DM4, PNU-159682, amanitin, duocarmycin, auristatin, maytansine, tubulysin, calicheamicin, SN-38, taxol, tubulysin, daunomycin, vinblastine, doxorubicine, methotrexate, pyrrolobenzodiazepine, pyrrole-based kinesin spindle protein (KSP) inhibitors, indolino-benzodiazepine dimers, or radioisotopes and/or pharmaceutically acceptable salts thereof;
   if more than one payload (P¹) is present, each P¹ is independently selected from the aforementioned moieties (i) to (iii) or (i) to (iv) of item 3, the payloads being preferably identical to each other.
7. The compound of any one of items 1 to 3, wherein at least one P¹ is a moiety derived from a compound comprising one or more polyethylene oxide groups, wherein preferably each polyethylene oxide group independently comprises from 4 to 600, more preferably from 10 to 200, even more preferably from 15 to 80 repeating units;
   P¹ being preferably a moiety represented by the following formula (12c):

—X⁶—(CH₂CH₂O)ₙ₁₉′—X⁷ (12c)
wherein,
n19′ is an integer of 4 to 600, preferably 10 to 200, more preferably 15 to 80;
X⁶ is selected from a single covalent bond, —(C═O)—, and —N(R)— in which R represents a hydrogen atom, an alkyl group or a cycloalkyl group;
X⁷ represents an alkyl group having 1 to 6 carbon atoms such as a methyl group, a carbonyl-containing group such an acetyl group or a group of formula —(CH₂)ₙ₄—CO₂H, a thiocarbonyl-containing group, a group of formula —(CH₂)ₙ₄OR, a group of formula —(CH₂)ₙ₄—SO₃H, or an amino-containing group such as a group of formula —(CH₂)ₙ₄—(C═X)—N(R′)(R) or —(CH₂)ₙ₄—N(R′)(R), in which X is O or S, R and R′ are each independently selected from a hydrogen atom, an alkyl group or a cycloalkyl group, and n4 is an integer of 1 to 6;
X⁷ being preferably —CH₃, or a group represented by the following formula (12a′):

—(CH₂)ₙ₅—(C═X)N(R)—(CH₂)ₙ₆—(C═X)N(H)(R) (12a′)
wherein,
each X is independently selected from O and S, preferably O,
each R is independently selected from a hydrogen atom, an alkyl group and a cycloalkyl group, and
n5 and n6 are each independently an integer of 1 to 6, preferably 1 or 2; and
X⁷ being most preferably —CH₃;
if more than one payload (P¹) is present, each P¹ is independently selected from the aforementioned moieties (i) to (iv) of item 3 and is more preferably a moiety of formula (12c) above, the payloads being preferably identical to each other.
8. The compound of any one of items 2 to 7, wherein the linker (L) is selected from
   (a1) an alkylene group having from 1 to 12 carbon atoms, preferably an alkylene group having from 2 to 6 carbon atoms such as a propylene group;
   (b1) a polyalkylene oxide group with 2 or 3 carbon atoms having from 1 to 36 repeating units; preferably a group represented by the formula

—NH—(CH₂CH₂O)ₙ₁—CH₂CH₂—
wherein n1 is an integer of 0 to 35, e.g. 1 to 20;
(c1) a peptidic group comprising 2 to 12 amino acids;
the linker (L) being preferably a group (b1) as defined above;
if the compound of formula (1) comprises more than one linker (L), each L is independently selected from the aforementioned groups (a1) to (c1), each linker (L) being preferably a group (b1).
9. The compound of any one of items 2 to 8, wherein the branching group (K) is represented by the formula *—CH(R₁—**)(R₂—**) (3a), or the following formula (3b),

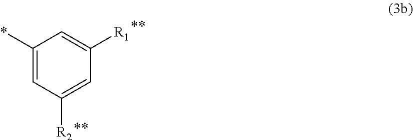

wherein,
R₁ and R₂ are each independently selected from the group consisting of —(CH₂)ₘ₁—** and —(CH₂)ₘ₁R₃—**;
R₃ is selected from the group consisting of —NH—**, —(C═X)R₄—** and —NH(C═X)R₄—**, and is preferably —NH—** or —NH(C═X)R₄—**;
R₄ is —(CH₂)ₘ₂—**, —(CH₂)ₘ₂S—**, —CH(R₇**)₂, —(CH₂)ₘ₂NH—**, or an aryl group of formula (3c):

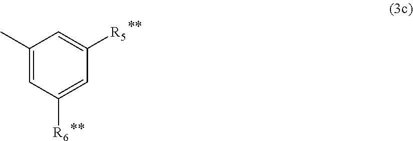

R₅ and R₆ are each independently selected from —(CH₂)ₘ₂—**, —(CH₂)ₘ₂S**—, —CH(CH₂S—**)₂, —(CH₂)ₘ₂NH—**, —CH(R₇—**)₂, —(CH₂)ₘ₂NH(C═X)R₈—**, and —CH₂(CH₂)ₘ₂H—**;
R₇ is —CH₂S—**, (CH₂)ₘ₂—**, or —(CH₂)ₘ₂R₉—*;
R₈ is —(CH₂)ₘ₃S—**;
R₉ is —NH(C═X)ₘ₃S—**;
each X is independently selected from O and S, and is preferably O;
* indicates covalent attachment to the reactive moiety (Y), optionally via a linker (L2);
** indicates covalent attachment to linker (L) in formula (2b), or to payload (P¹) in formula (2c);
m1, m2 and m3 are each independently selected from 0, 1, 2 and 3, with the proviso that if K is of formula (3a) m1 is not 0; each m1, m2 and m3 being preferably 1.
10. The compound of any one of items 2 to 9, wherein the branching group (K) is represented by one of the following formulae (3d) to (31):

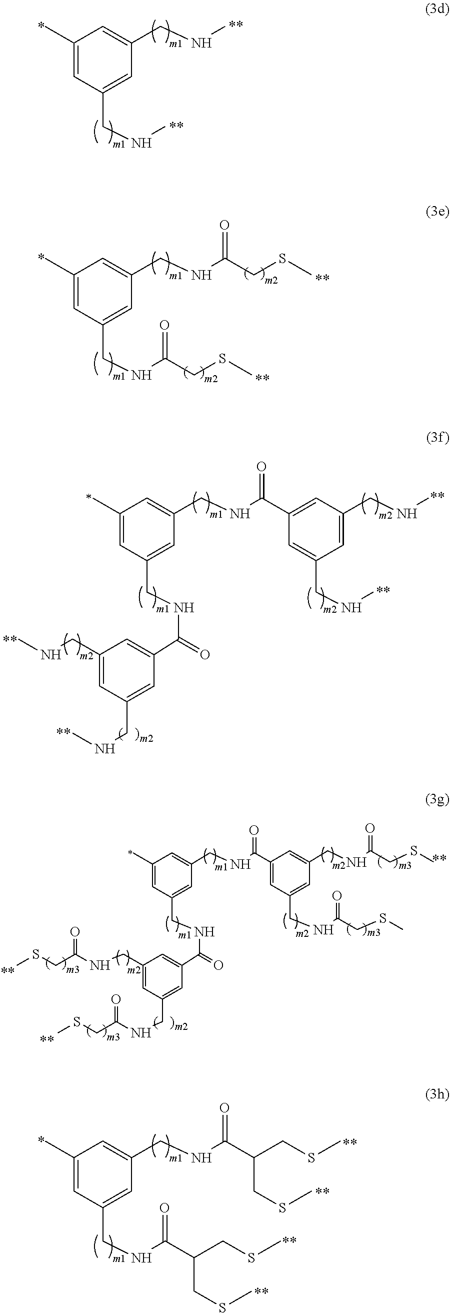

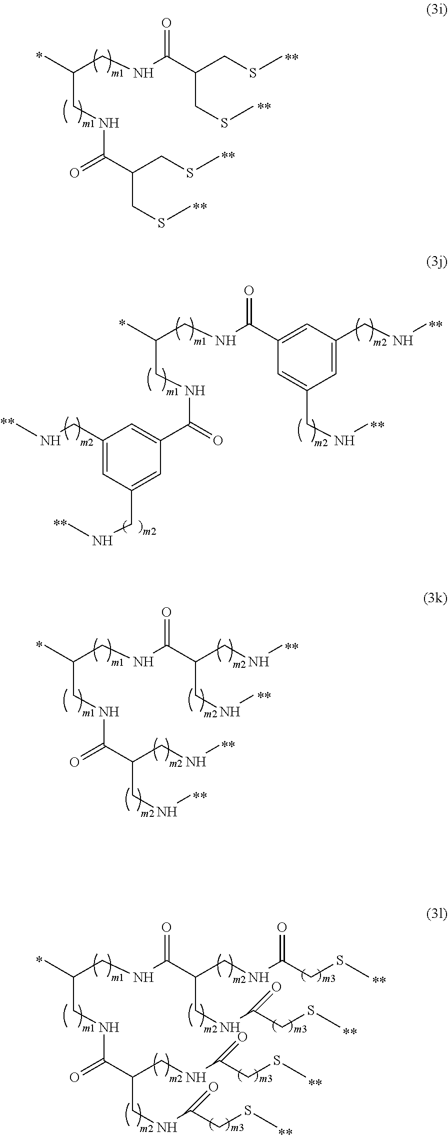

wherein
m1 is 0, 1, 2 or 3, preferably 1;
m2 is 1, 2, 3 or 4, preferably 1;
m3 is 0, 1, 2 or 3, preferably 1;
   * indicates covalent attachment to the reactive moiety (Y); and
   ** indicates covalent attachment to linker (L) or payload (P¹); the branching group (K) being preferably represented by formula (3d).
11. The compound of any one of items 2 to 8, wherein the branching group (K) is represented by the following formula (3m):

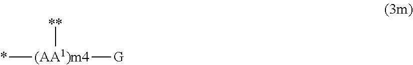

wherein,
m4 is an integer of 1 to 10, preferably 1 to 6, more preferably 1, 2 or 3;
each AA1 is independently a moiety derived from a trifunctional amino acid, such as a diamino-carboxylic acid, wherein preferably each AA1 is independently a moiety derived from Orn, Lys, Dab or Dap, more preferably from Orn or Lys; the side chain originating from the trifunctional amino acid is covalently attached to a linker (L) in formula (2b) or a payload (P¹) in formula (2c);
G is absent, or represents one selected from:
   a hydrogen atom,
   a group of formula —N(H)(R), wherein R is selected from a hydrogen atom, an alkyl group and a cycloalkyl group; and
   a group of formula —N(H)(R)-(CH₂)ₙ₁₃—(C═O)N(H)(R), wherein each R is independently selected from a hydrogen atom, an alkyl group and a cycloalkyl group, and n13 is an integer of 1 to 6, preferably 1 or 2;
If G is absent, the resulting free valence forms a covalent bond to a linker (L) in formula (2b) or to a payload (P¹) in formula (2c);
* indicates covalent attachment to the reactive moiety (Y); and
** indicates covalent attachment to linker (L) or payload (P¹); with the proviso that if m4 is 1, G is absent.
12. The compound of any one of items 1 to 11, wherein the reactive moiety (Y) is represented by one of the following formulae (4a) and (4b):

*′-F1-RC-F2-*** (4a)

*′-(F1-RC-F2)-M-*** (4b)
wherein,
RC is a reactive centre, preferably an electrophilic reactive centre, and more preferably a group selected from C═O and C═S;
F1 is a single covalent bond, an atom, or a group of atoms; preferably a CH₂ or NH group of atoms or an atom selected from O and S, or a group of atoms comprising one or more atoms selected from C, N, O, and S; more preferably a CH₂ group of atoms or an atom selected from O and S;
F2 represents an atom, or a group of atoms; preferably an atom selected from O, and S, or a group of atoms comprising one or more atoms selected from C, N, O, and S; more preferably an atom selected from O and S;
M is a group capable of modulating the electron density and stability of F2, preferably a group capable of withdrawing electrons;
*′ indicates covalent attachment to the group (P); and
*** indicates covalent attachment to the peptide (V);
the reactive moiety (Y) being preferably represented by formula (4b).
13. The compound of item 12, wherein the group (M) capable of modulating the electron density and stability of F2 is represented by the following formula (4c):

***‘--M’-B-E--*** (4c)
wherein,
M′ is a moiety derived from succinimide or an aryl group having 6, 10 or 14 ring members and 1, 2 or 3 condensed rings, respectively, or a heteroaryl group having 5 to 20 ring members, 1, 2 or 3 condensed rings and 1 to 4 heteroatoms independently selected from N, O and S, each group being optionally substituted with one or more substituents; preferably a divalent group derived from a phenyl group, a naphthyl group, a pyridyl group, a quinolinyl group, an isoquinolinyl group or a benzotriazolyl group, each group being optionally substituted with one or more substituents and each substituent being preferably selected from —F, —Br, —Cl, —I, —NO₂, —CN, —C₁₋₆-alkyl, —C₁₋₆-alkoxy, —C₁₋₆-amido such as —C(O)NH₂, and combinations thereof such as —CCl₃, —CF₃ or —CH₂NO₂;
B is selected from:
   a single covalent bond, O, S, or a group of atoms NR′, wherein R′ represents a hydrogen atom, —OH, an alkyl group, a cycloalkyl group, a C₂₋₆-alkenylene group, or a C₂₋₆-alkynylene group;
   a group represented by the general formula (4d):

♦—X¹—(CH₂CH₂O)ₙ₂—CH₂CH₂—^{♦♦} (4d)
wherein
X¹ is —C(═O)NH—, —C(═O)—, —NH— or —S—; preferably —C(═O)NH—;
n2 is an integer of 1 to 24, preferably 1 to 10, more preferably 1 to 3;
♦ indicates covalent attachment to M′, and
♦♦ indicates covalent attachment to E;
a peptidic group having 6 to 25 amino acids in the main chain, e.g. 9 amino acids in the main chain, each amino acid being preferably selected from Pro, Gly, Ala, Asn, Asp, Thr, Glu, Gln and Ser; more preferably from Pro, Gly and Ser;
and any combinations thereof;
wherein, B is preferably a single covalent bond or a group having the general formula (4d), more preferably a single covalent bond;
E is C═O, C═S or C(═NR″), wherein R″ represents a hydrogen atom, OH, an alkyl group, a cycloalkyl group, S═O, or S(═O)₂; preferably E is C═O;
***′ indicates covalent attachment to F2, and
*** indicates covalent attachment to the peptide (V).
14. The compound of item 12 or 13, wherein the moiety (F1-RC-F2) is represented by one of the following formulae (4a′) to (41′) and/or the group (M) is represented by one of the following formulae (5a) to (5i′):

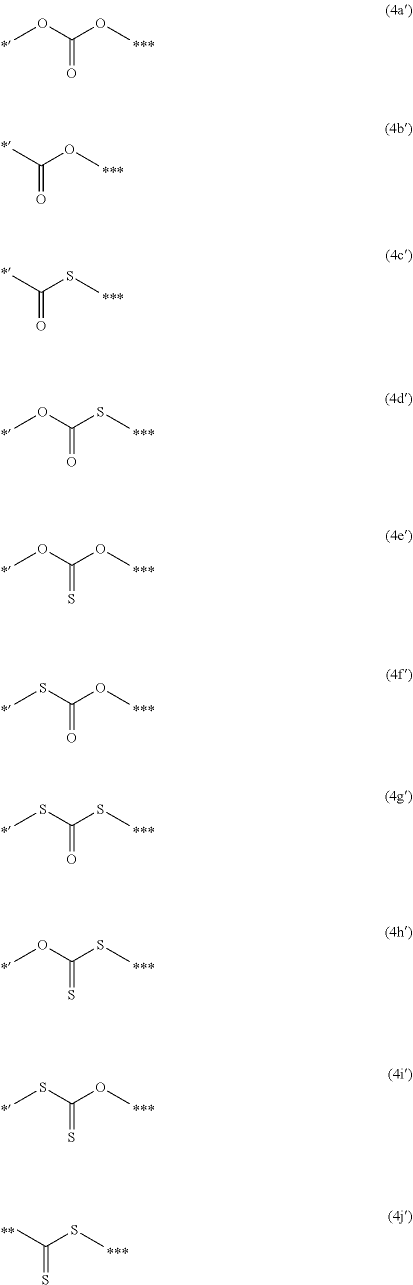

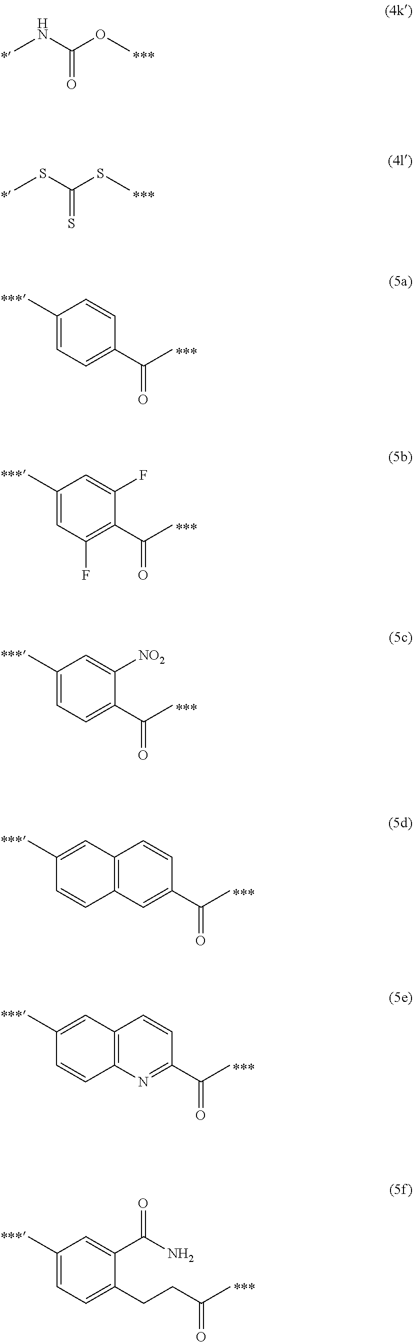

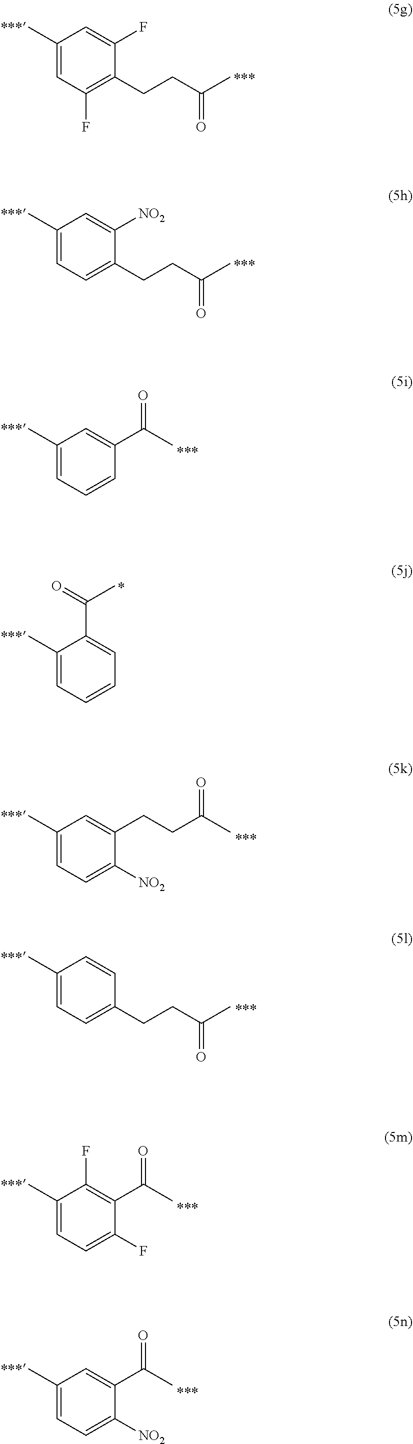

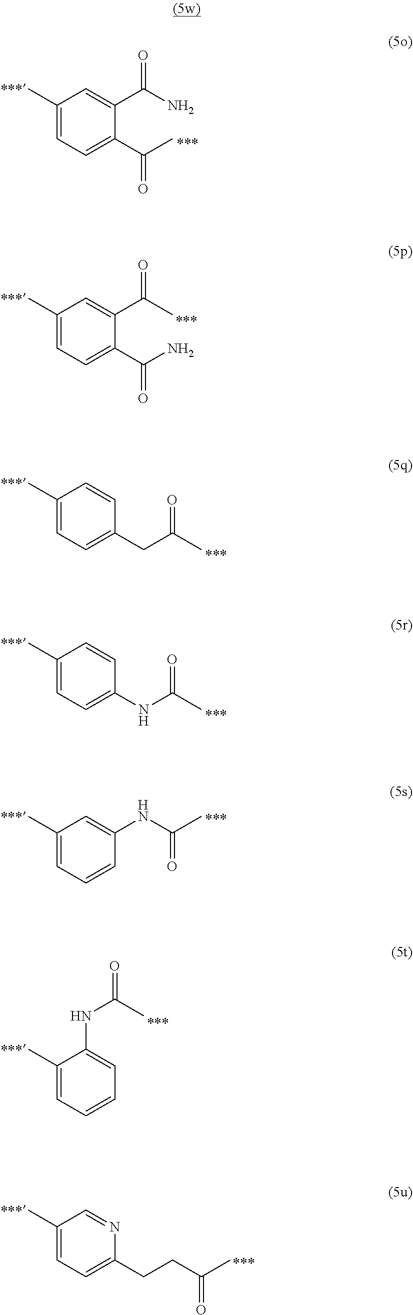

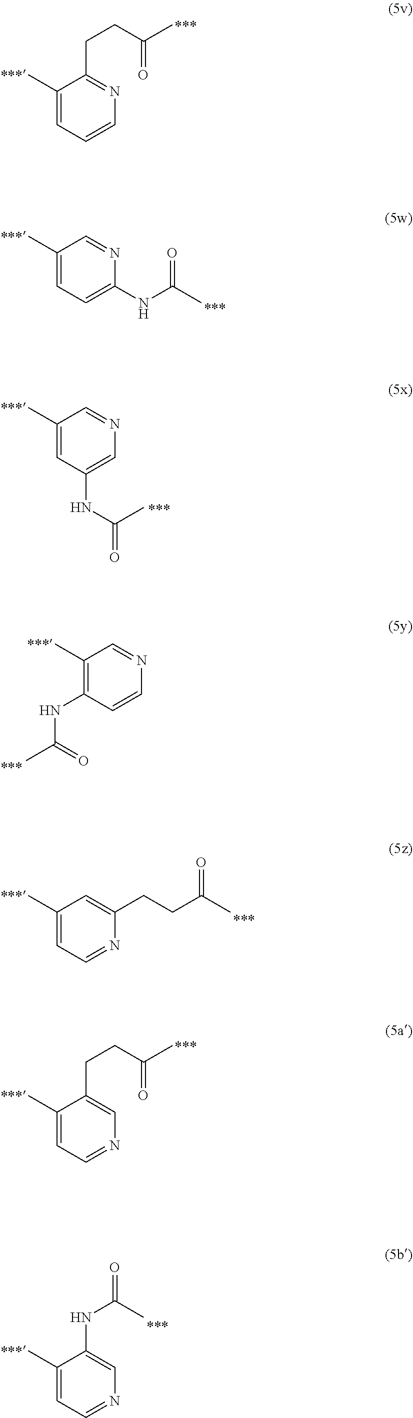

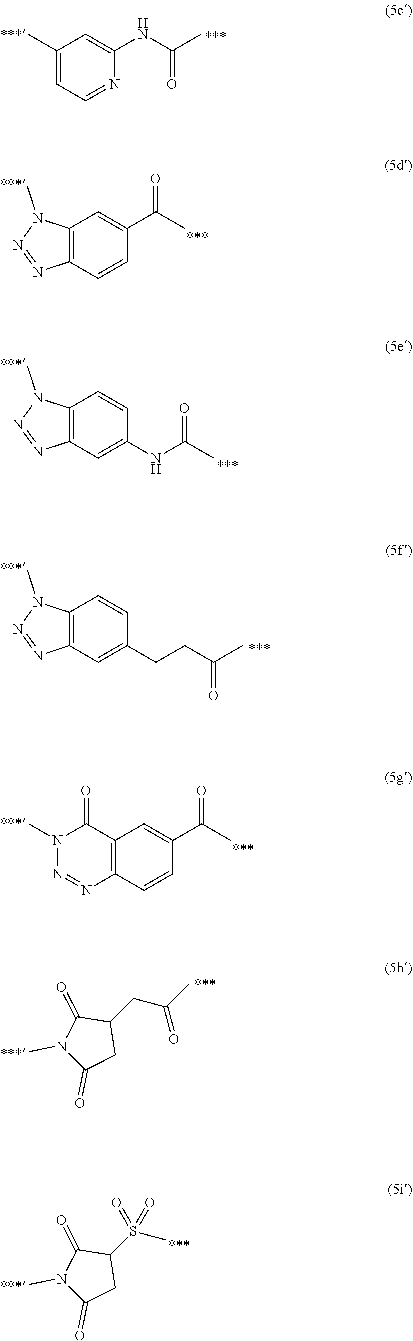

wherein,
*′ indicates covalent attachment to the group (P),
*** indicates covalent attachment to the peptide (V), or to the group (M) if M is present, and
***′ indicates covalent attachment to F2 of the reactive moiety (Y).
15. The compound of any one of items 1 to 14, wherein the reactive moiety (Y) is represented by one of the following formulae (6a) to (6l′):

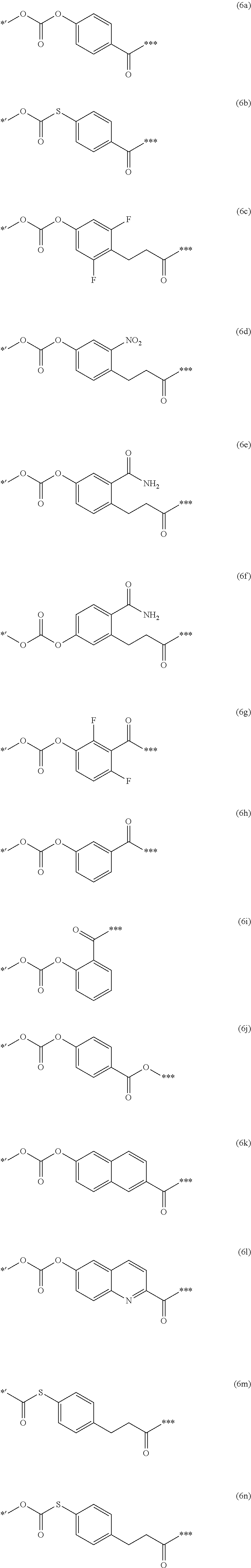

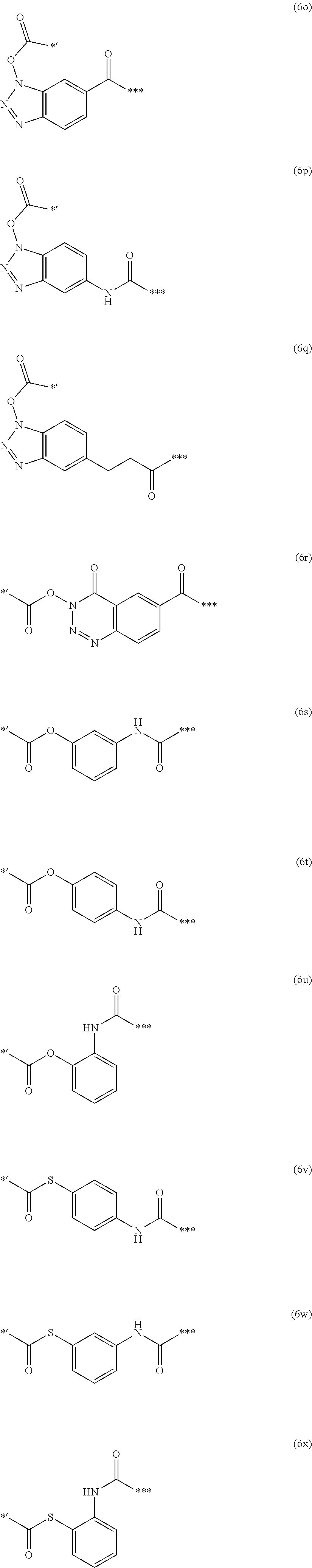

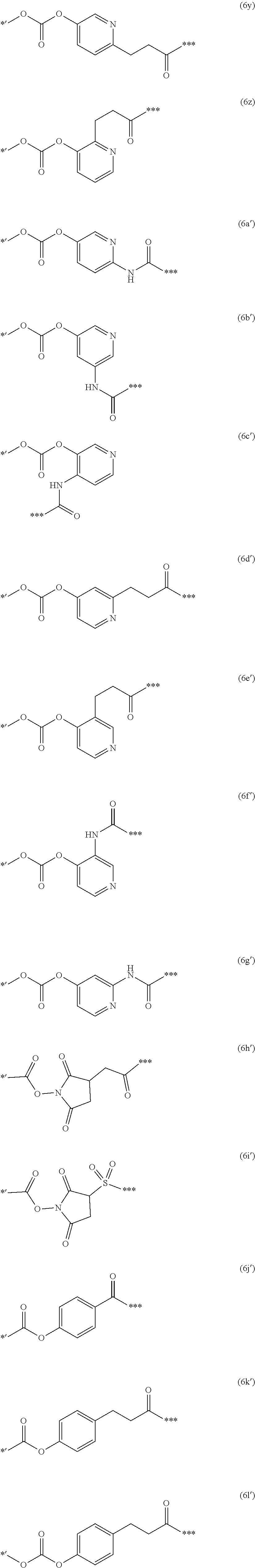

wherein,
indicates covalent attachment to the group (P); and
*** indicates covalent attachment to the peptide (V).
16. The compound of any one of items 1 to 15, wherein the peptide (V) comprises a sequence of 11 to 17 amino acids, e.g. 13 to 17 amino acids, which is preferably cyclic, and more preferably represented by the following formula (7a):

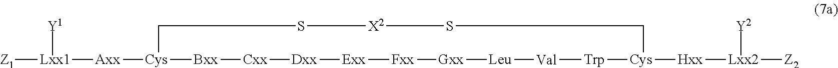

wherein,
   Bxx represents an amino acid selected from an amino acid with an amino group-containing side chain, Ala, Tyr, homo-tyrosine (hTyr), and meta-tyrosine (mTyr); preferably an amino acid selected from Lys, homo-lysine (hLys), ornithine (Orn), 2,3-diaminopropionic acid (Dap), 2,4-diaminobutyric acid (Dab), Ala, Tyr, hTyr, and mTyr; more preferably Ala;
   Cxx, Dxx, Exx, Fxx, Gxx each independently represent an amino acid;
   Axx represents an amino acid, a dicarboxylic acid, or a peptide moiety represented by the following formula (8a):

---Axx1-Axx2-Axx3--- (8a)
wherein, in formula (8a),
Axx1 represents a single covalent bond, or an amino acid such as Arg;
Axx2 represents an amino acid such as Gly or Cys, preferably Gly; and
Axx3 represents an amino acid such Asp or Asn;
Hxx represents an amino acid, or a peptide moiety represented by the following formula (8b):

---Hxx1-Hxx2-Hxx3--- (8b)
wherein, in formula (8b),
Hxx1 represents an amino acid such as Thr;
Hxx2 represents a single covalent bond or an amino acid such as Tyr or Cys; and
Hxx3 represents a single covalent bond, or an amino acid such as His; and the side chain of Axx2 may be covalently attached to the side chain of Hxx2 to form a ring;
wherein preferably both Hxx2 and Hxx3 represent single covalent bonds;
if Axx2 is Cys and Hxx2 is Cys, preferably the side chains of Axx2 and Hxx2 are linked together to form a group of formula —(S—X³—S)—, wherein X³ represents a single covalent bond or a divalent group comprising one or more atoms selected from carbon, nitrogen and oxygen such as a divalent maleimide group, a divalent acetone group or a divalent arylene group; preferably X³ represents a single covalent bond;
Lxx1 and Lxx2 each independently represent a single covalent bond or a trifunctional amino acid such as a diamino-carboxylic acid; with the proviso that at least one of Lxx1 and Lxx2 is a single covalent bond;
Z₁ represents a group selected from:
   a group covalently attached to the N-terminus of Axx, if Lxx1 is a single covalent bond, which is selected from a hydrogen atom, a carbonyl-containing group such as an acetyl group, a C₂₋₃ polyalkylene oxide-containing group, and a group derived from a compound containing a conjugation group such as biotin, DBCO, TCO, TZ, BCN, an alkyne, an azide, a bromoacetamide, a maleimide and a thiol, wherein said conjugation group is optionally attached via a spacer (S¹);
   a group covalently attached to the N-terminus of Lxx1, if Lxx1 is a trifunctional amino acid and Y¹ is attached to the side chain of Lxx1, which is selected from a hydrogen atom, a carbonyl-containing group such as an acetyl group, and a C₂₋₃ polyalkylene oxide-containing group; and
   a hydrogen atom or a C₂₋₃ polyalkylene oxide-containing group covalently attached to the side chain of Lxx1, if Lxx1 is a trifunctional amino acid and Y¹ is covalently attached to the N-terminus of Lxx1;
Z₂ represents a group selected from:
   a group covalently attached to the C-terminus of Hxx, if Lxx2 is a single covalent bond, which is selected from —N(H)(R) wherein R represents a hydrogen atom, an alkyl or cycloalkyl group, a C₂₋₃ polyalkylene oxide-containing group, and a group derived from a compound containing a conjugation group such as biotin, DBCO, TCO, TZ, BCN, an alkyne, an azide, a bromoacetamide, a maleimide or a thiol, wherein said conjugation group is optionally attached via a spacer (S¹);
   a group covalently attached to the C-terminus of Lxx2, if Lxx2 is a trifunctional amino acid and Y² is covalently attached to the side chain of Lxx2; preferably a C₂₋₃ polyalkylene oxide-containing group, a group represented by N(H)(R) wherein R represents a hydrogen atom, an alkyl group, or a cycloalkyl group; and
   a hydrogen atom or a C₂₋₃ polyalkylene oxide-containing group, covalently attached to the side chain of Lxx2, if Lxx2 is trifunctional amino acid and Y² is covalently attached to the C-terminus of Lxx2;
Y¹ represents a moiety that is only present if Lxx1 is a trifunctional amino acid, wherein Y¹ is covalently attached to the side chain of Lxx1 if Z₁ is attached to the N-terminus of Lxx1, or to the N-terminus of Lxx1 if Z₁ is attached to the side chain of Lxx1;
   wherein Y¹ is derived from a compound containing a conjugation group, which is preferably selected from biotin, DBCO, TCO, TZ, BCN, an alkyne, an azide, a bromoacetamide, a maleimide, and a thiol, and wherein said conjugation group is optionally attached via a spacer (S¹);
Y² represents a moiety that is only present if Lxx2 is a trifunctional amino acid, wherein Y² is covalently attached to the side chain of Lxx2 if Z₂ is attached to the C-terminus of Lxx2, or to the C-terminus of Lxx2 if Z₂ is attached to the side chain of Lxx2;
   wherein Y² is derived from a compound containing a conjugation group, which is preferably selected from biotin, DBCO, TCO, TZ, BCN, an alkyne, an azide, a bromoacetamide, a maleimide, and a thiol, and wherein said conjugation group is optionally attached via a spacer (S¹);
S¹ is a spacer represented by the following formula (8c):

α-X⁴—(CH₂CH₂O)ₙ₂—CH₂CH₂—X⁵-β (8c)
wherein
X⁴ is NH, O or S; preferably NH;
X⁵ is NH or C═O, preferably C═O if X⁵ is covalently attached to the peptide (V);
n2 is an integer of 1 to 46, preferably 1 to 24, most preferably 1 to 12; and
α indicates covalent attachment to Y¹ or Y² if X⁵ is covalently attached to the peptide (V), or covalent attachment to the peptide (V) if X5 is covalently attached to Y¹ or Y²; and
β indicates covalent attachment to the peptide (V) if X⁴ is covalently attached to Y¹ or Y², or covalent attachment to Y¹ or Y² if X⁴ is covalently attached to the peptide (V);
X² represents a single covalent bond or a divalent group comprising one or more atoms selected from carbon, nitrogen and oxygen such as a divalent maleimide group, a divalent acetone group or a divalent arylene group; preferably a single covalent bond;
wherein at least one of Axx, Bxx, Cxx, Dxx, Exx, Fxx, Gxx and Hxx, preferably one or more of Bxx, Dxx, Exx, Fxx, and Gxx, more preferably one or more of Bxx, Exx, and Gxx, and most preferably Bxx and/or Exx, represents an amino acid with an amino group-containing side chain, Tyr, hTyr, or mTyr, preferably an amino acid with an amino group-containing side chain, more preferably an amino acid selected from Lys, hLys, Orn, Dap, and Dab, which is covalently attached to the reactive moiety (Y) via its side chain; with the proviso that the peptide (V) does not comprise more than three amino acids with an amino group-containing side chain; and
if the reactive moiety (Y) is attached to the peptide (V) via a Tyr, hTyr, or mTyr, the reactive moiety (Y) is preferably a moiety of formula (4a) as defined in item 10.
17. The compound of item 16, wherein at least one of Axx, Cxx, Dxx, Exx, Fxx, Gxx, Hxx, Lxx1 and Lxx2 is defined as follows:
   Axx represents an amino acid selected from Ala, 2,3-diamino-propionic acid (Dap), Asp, Glu, 2-amino suberic acid, α-amino butyric acid, Asn and Gln, a dicarboxylic acid selected from succinic acid, glutaric acid and adipic acid; preferably Ala, Asp or Asn; more preferably Asp; or a peptide moiety of formula (8a), wherein Axx1 is a single covalent bond, Axx2 is Cys, and Axx3 is Asp;
   Cxx represents an amino acid selected from Trp, Phe, Tyr, phenylglycine (Phg), 3-benzothiopen-2-yl-L-alanine, 3-naphthalen-2-yl-L-alanine, 3-biphenyl-4-yl-L-alanine and 3-naphthalen-1-yl-L-alanine; preferably Trp;
   Dxx represents an amino acid selected from His, Ala, 3-pyridin-2-yl-L-alanine, mTyr and Phe; preferably His, Ala or mTyr; more preferably His;
   Exx represents an amino acid selected from Ala, 2-amino-butyric acid (Abu), Gly, Leu, Ile, Val, Met, cyclohexyl alanine (Cha), Phe, Thr, Cys, Tyr, and norleucine (Nle); preferably Ala, Nle or Leu; more preferably Leu;
   Fxx represents an amino acid selected from Ala, Gly, Asn, Ser, Abu, and Asp; preferably Ala or Gly; more preferably Gly;
   Gxx represents an amino acid selected from Ala, Glu, Asp, Gln, His, Arg, Ser, and Asn; preferably Asp or Glu; more preferably Glu;
   Hxx represents an amino acid selected from Thr, Ser, Ala, Asn, Val, Abu, Ile, Met, Leu, Pro, Gln, and Cys; preferably Thr or Ser; more preferably Thr; or a peptide moiety of formula (8b), wherein Hxx1 is Thr, Hxx2 is Cys, and Hxx3 is a single covalent bond;
   Lxx1 and Lxx2 each independently represent an amino acid selected from Dap, Dab, Lys, Orn and hLys, preferably an amino acid selected from Dap, Dab, Lys, Orn and hLys; and
   if Dxx or Exx represents an amino acid with an amino group-containing side chain, Tyr, hTyr, or mTyr, which is covalently attached to the reactive moiety (Y) via its side chain, Gxx is preferably Glu, Gln, His, Arg or Asn, and more preferably Gln.
18. The compound of any of items 1 to 17, wherein the peptide (V) is represented by the following formula (9a):

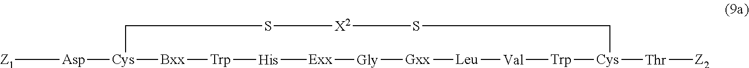

wherein,
   Z₁, Z₂, Bxx, Exx, Gxx and X² are as defined in item 16; and
   at least one of Bxx, Exx and Gxx, preferably Bxx and/or Exx, represents an amino acid with an amino-group containing side chain, Tyr, hTyr, or mTyr; preferably an amino acid with an amino group containing side chain; and more preferably an amino acid selected from Lys, hLys, Orn, Dap, and Dab; said amino acid being covalently bonded to the reactive moiety (Y) via its side chain; and
   if Exx represents an amino acid with an amino group-containing side chain, Tyr, hTyr, or mTyr, preferably an amino acid with an amino group containing side chain; and more preferably an amino acid selected from Lys, hLys, Orn, Dap, and Dab, which is covalently attached to the reactive moiety (Y) via its side chain, Gxx is preferably Glu, Gln, His, Arg or Asn, and more preferably Gln;
and preferably one or two of Bxx, Exx and Gxx is defined as follows:
   Bxx represents an amino acid selected from an amino acid with an amino group-containing side chain, Ala, Tyr, hTyr, and mTyr; preferably Lys, hLys, Orn, Dap, Dab, Ala, Tyr, hTyr, or mTyr; more preferably Ala;
   Exx represents an amino acid selected from Ala, Abu, Gly, Leu, Ile, Val, Met, Cha, Phe, Thr, Cys, Tyr, and Nle; preferably Ala, Nle or Leu; more preferably Leu; and
   Gxx represents an amino acid selected from Ala, Glu, Asp, Gln, His, Arg, Ser, and Asn; preferably Asp or Glu; more preferably Glu.
19. The compound of any one of items 1 to 18, wherein the peptide (V) is represented by one of the following formulae (10a) to (10v), (10b′) and (10g′):

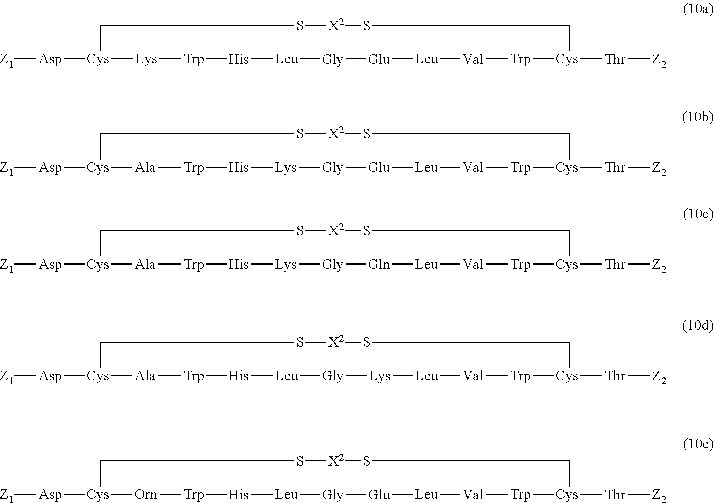

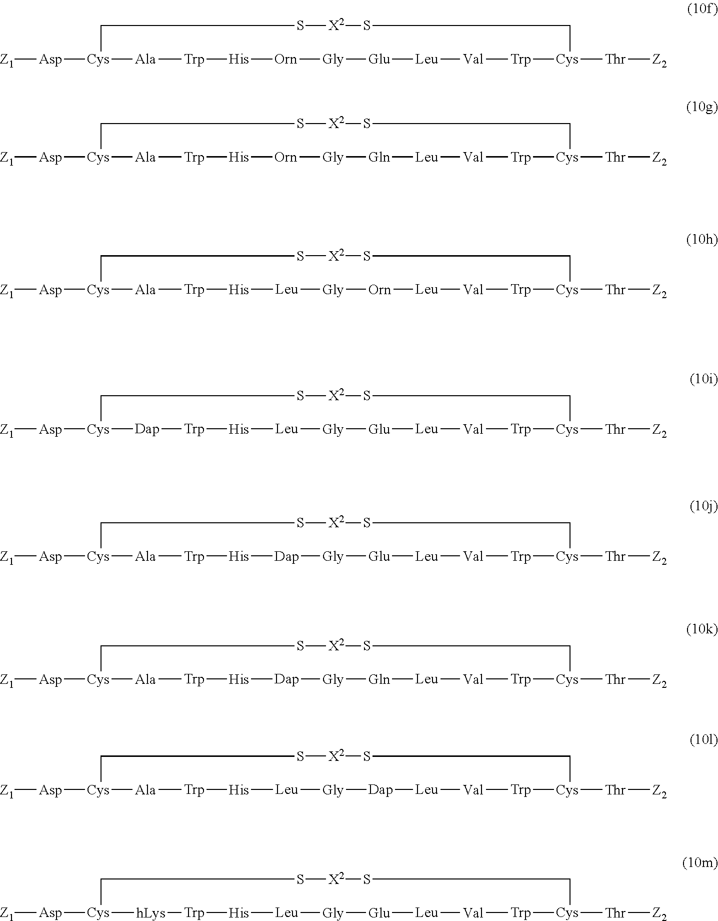

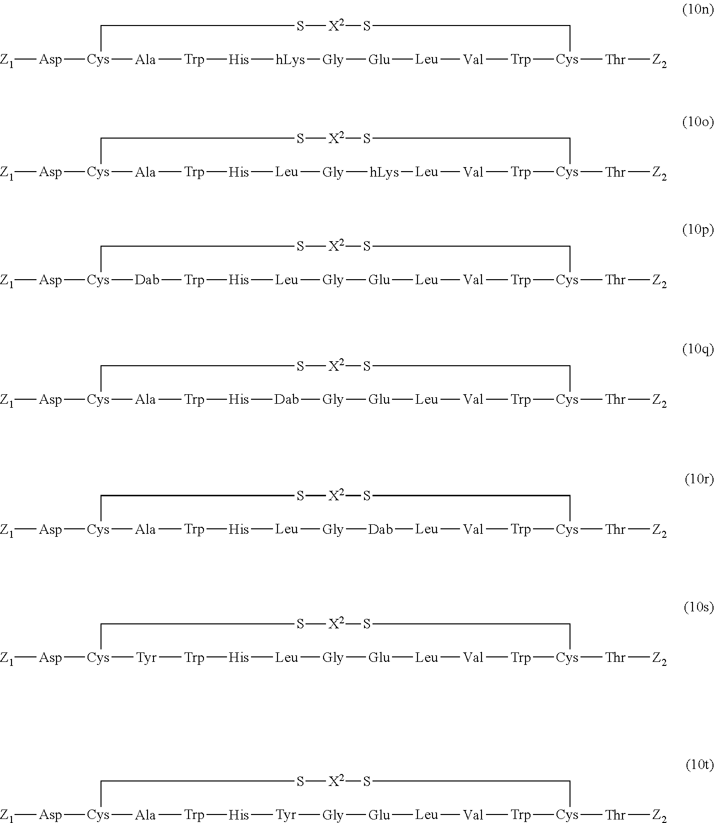

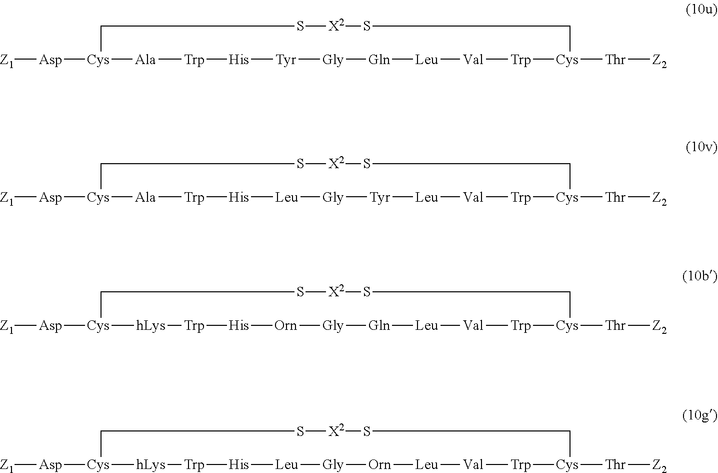

wherein, Z₁, Z₂, and X² are as defined in item 14; and
in the formulae (10a) to (10v), (10b′) and (10g′), the peptide (V) is covalently attached to the reactive moiety (Y) via the side chain of Tyr, Lys, hLys, Orn, Dap, or Dab comprised in V;
the peptide (V) being preferably represented by any one of the formulae (10a), (10b), (10b′) (10c), (10e), (10f), (10g), (10g′) (10h), (10i), (10j), (10k), (10m), (10n), (10p), (10q), (10s), (10t) and (10u), more preferably by any one of the formulae (10e), (10f), (10g), (10h), (10i), (10j), (10k), (10t) and (10u), and even more preferably by any one of the formulae (10e), (10f), (10g), (10h), (10i), (10j), and (10k).
20. The compound of any one of items 16 to 19, wherein at least one of Z₁ and Z₂ is a C₂₋₃ polyalkylene oxide-containing group, preferably a polyethylene oxide-containing group, which preferably comprises from 4 to 600, more preferably from 10 to 200, even more preferably from 15 to 80 repeating units;
   wherein,
   Z₁ is preferably represented by the following formula (13a):

X⁸—(OCH₂CH₂)ₙ₇—X⁹— (13a)
wherein
X⁸ represents an alkyl group having 1 to 6 carbon atoms such as an ethyl group, or an amino-containing group, preferably a group of formula —(CH₂)ₙ₈—N(H)(R), in which R is selected from a hydrogen atom, an alkyl group, a cycloalkyl group and a carbonyl-containing group such as an acetyl group, and n8 is an integer of 1 to 6, preferably 2;
X⁹ represents —(C═X)—, X being selected from O and S, preferably O; and n7 is an integer of 4 to 100, preferably 10 to 80, more preferably 15 to 40, most preferably 20 or 24;
and/or
Z₂ is preferably represented by the following formula (13b):

—X¹⁰—(CH₂CH₂O)ₙ₉—X¹¹ (13b)
wherein
n9 is an integer of 4 to 100, preferably 10 to 80, more preferably 15 to 40;
X¹⁰ is a single covalent bond, NH, O or S; preferably NH;
X¹¹ represents an alkyl group having 1 to 6 carbon atoms such as a methyl group, a carbonyl-containing group such an acetyl group or a group of formula —(CH₂)ₙ₁₀—CO₂H, a thiocarbonyl-containing group, a group of formula —(CH₂)ₙ₁₀OR, a group of formula —(CH₂)ₙ₁₀—SO₃H, or an amino-containing group such as a group of formula —(CH₂)ₙ₁₀—(C═X)—N(R′)(R) or —(CH₂)ₙ₁₀—N(R′)(R), in which X is O or S, R and R′ are each independently selected from a hydrogen atom, an alkyl group or a cycloalkyl group, and n10 is an integer of 1 to 6;
X¹¹ being preferably a methyl group, or a group represented by —CH₃ or the following formula (13b′):

—(CH₂)ₙ₁₁—(C═X)N(R)—(CH₂)ₙ₁₂—(C═X)N(H)(R) (13b′)
wherein
each X is independently selected from O and S, preferably O;
each R is independently selected from a hydrogen atom, an alkyl group and a cycloalkyl group; and
n11 and n12 are each independently an integer of 1 to 6, preferably 1 or 2, more preferably 2;
X¹¹ being most preferably-CH₃.
21. The compound of item 20, wherein Z₁ is a polyethylene oxide-containing group comprising from 10 to 200, preferably from 15 to 80 repeating units, more preferably a group of formula (13a) as defined in item 20, and Z₂ is a group represented by —N(H)(R) wherein R represents a hydrogen atom, an alkyl group, or a cycloalkyl group; and
   wherein the peptide (V) is preferably represented by any one of the formulae (10e), (10f), (10g), (10h), (10i), (10j), (10k), (10t) and (10u), more preferably by any one of the formulae (10e), (10f), (10g), (10h), (10i), (10j), and (10k), and even more preferably by formula (10f).
22. The compound of any one of items 1 to 21, which is selected from the group consisting of

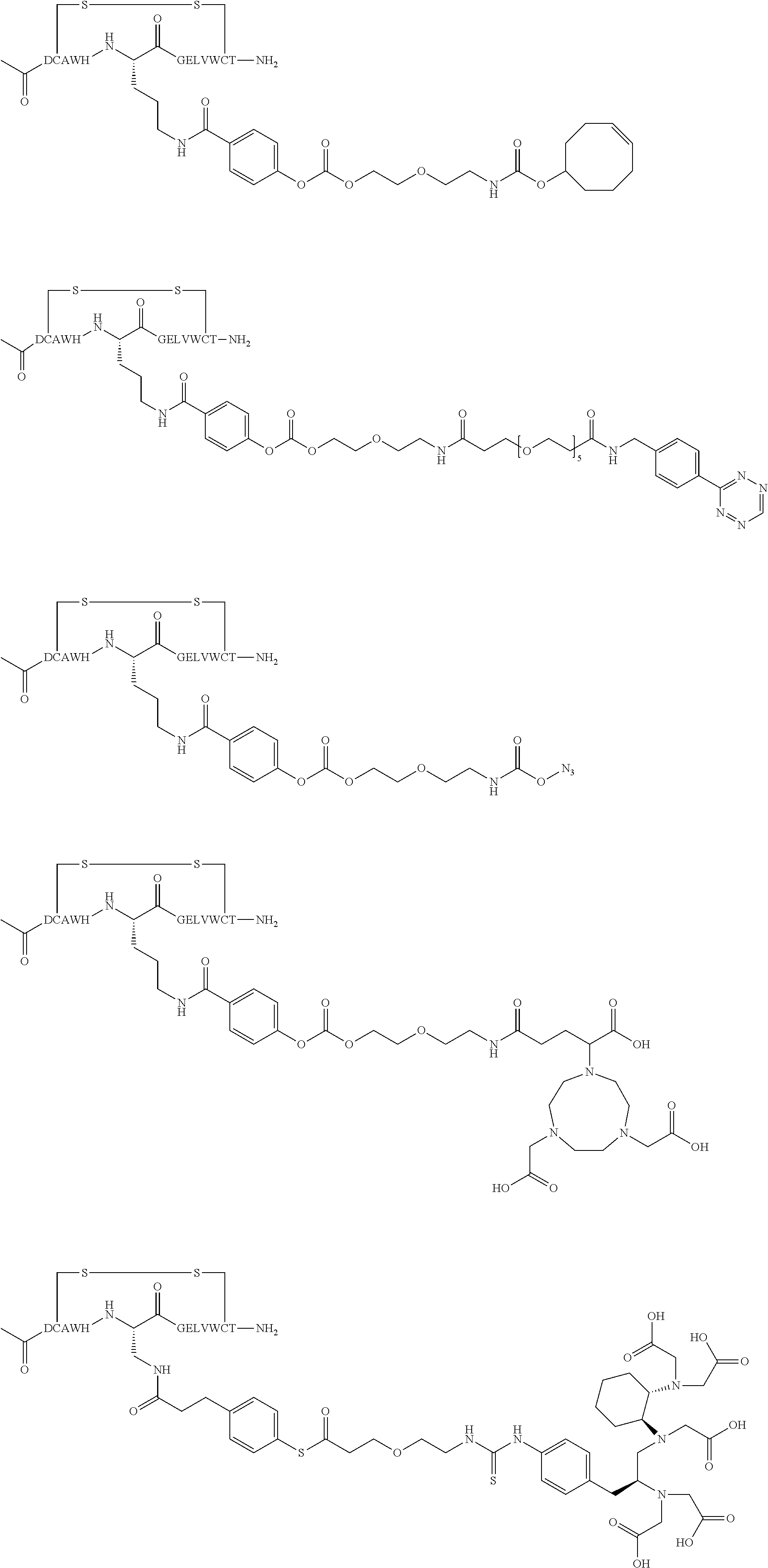

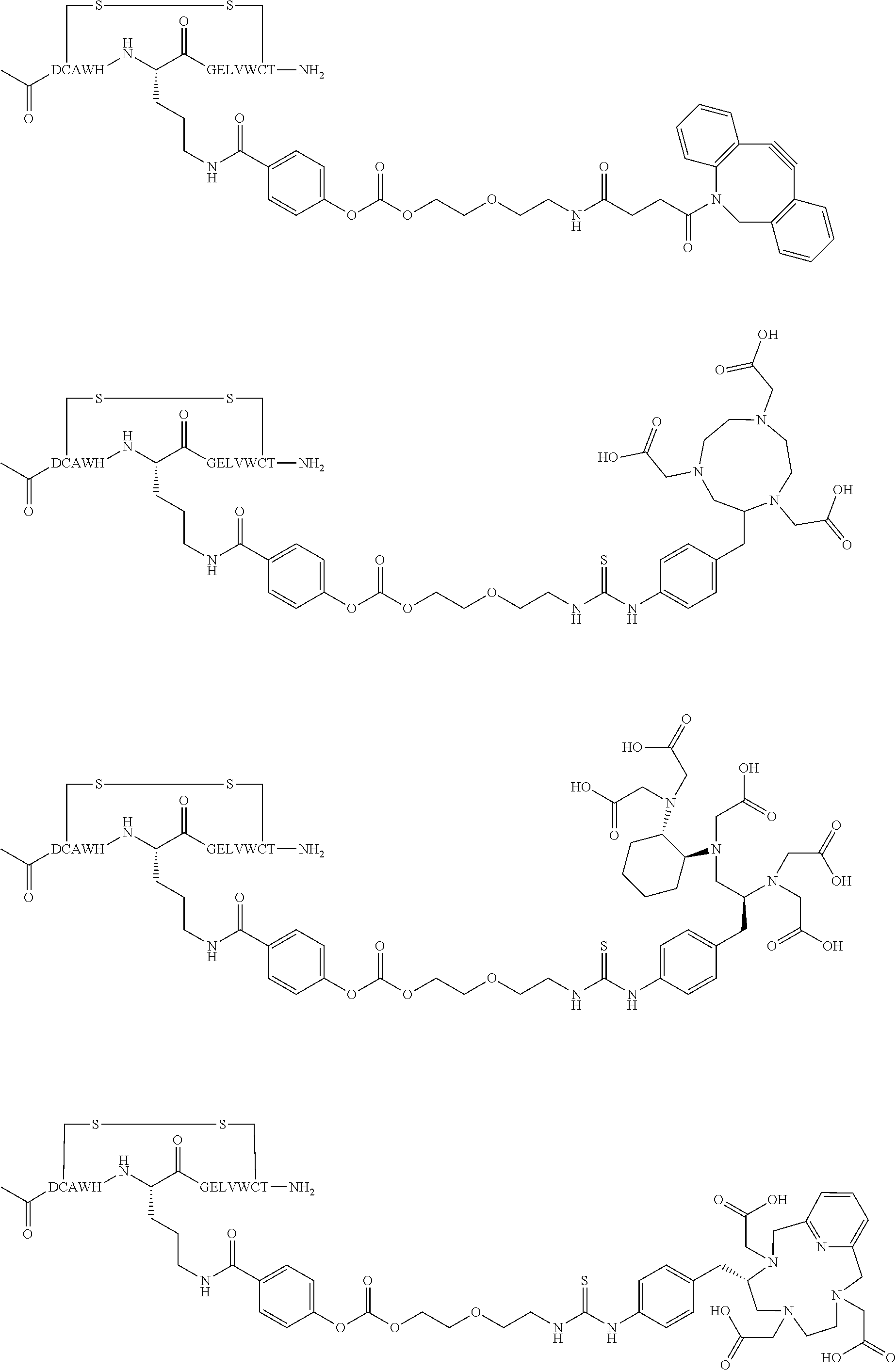

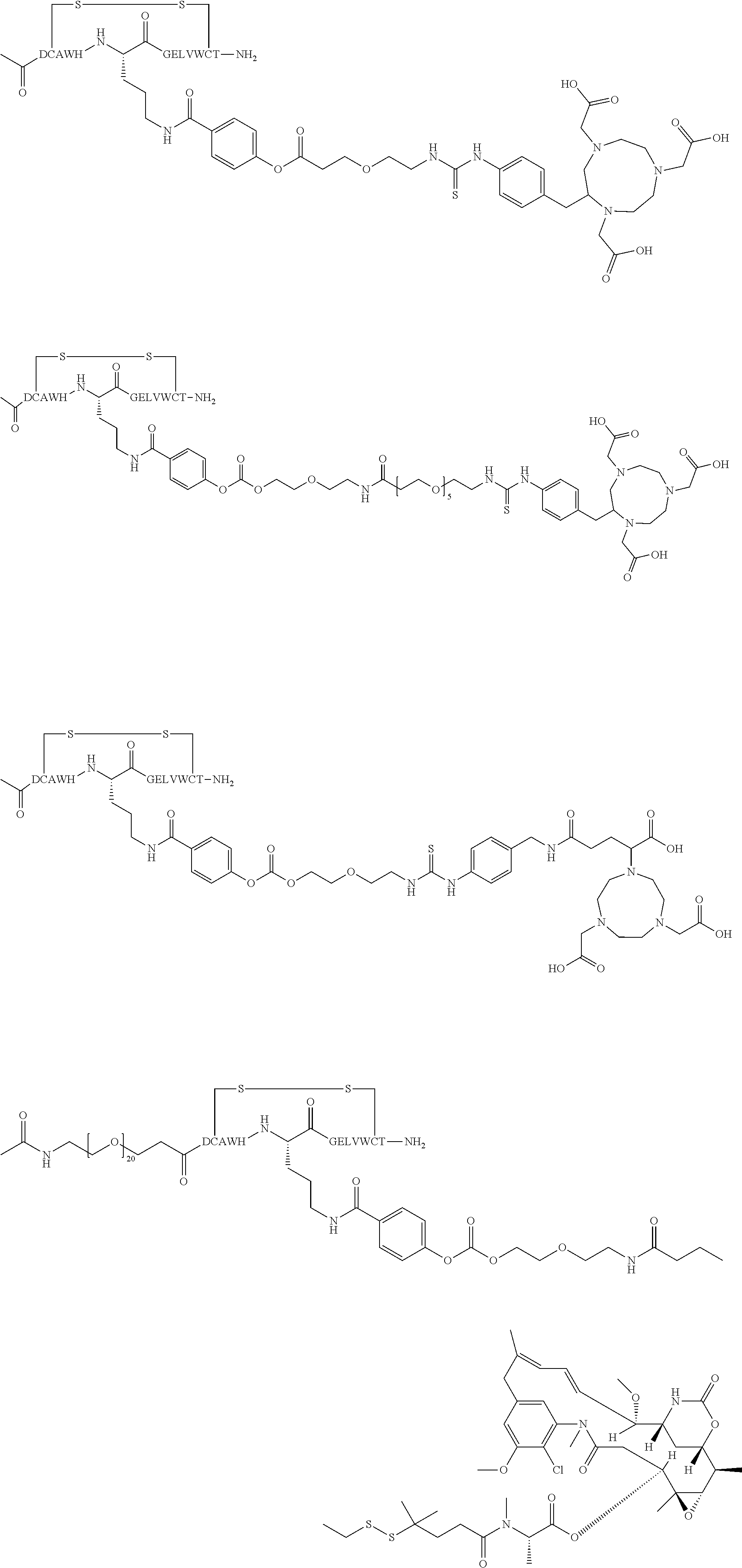

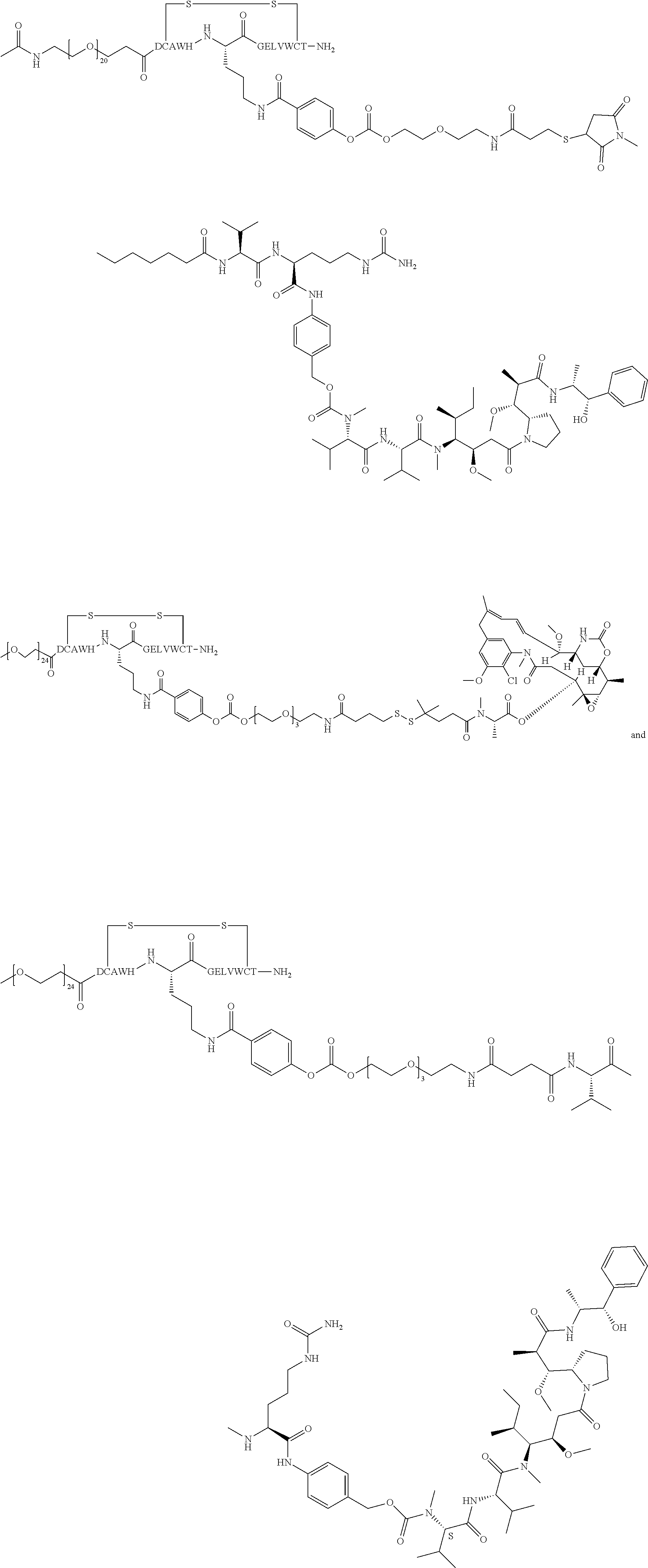

23. Kit for the regioselective modification of an antibody or fragment thereof, the antibody fragment being optionally incorporated into an Fc-fusion protein, comprising the compound of any of items 1 to 22 and a buffer; wherein the buffer has preferably a pH of 5.5 to 11, more preferably 7.0 to 9.5.
24. The kit for the regioselective modification of an antibody or fragment thereof of item 23, wherein the compound is immobilized on a solid phase matrix, preferably wherein the compound is immobilized on the solid support via a biotin-streptavidin interaction, via a covalent linkage obtained by click reaction between an alkyne and azide, via covalent linkage obtained by reaction between a thiol and an acetamide, via covalent linkage obtained by reaction between a derivative of TCO and a derivative of TZ, or via covalent linkage obtained by reaction between a thiol and maleimide.
25. Method for the regioselective modification of an antibody or fragment thereof, the antibody fragment being optionally incorporated into an Fc-fusion protein, comprising reacting an antibody or fragment thereof with a compound according to any of items 1 to 22.
26. The method of item 25, wherein
   the antibody is a monoclonal antibody, preferably an antibody selected from the group consisting of adalimumab, aducanumab, alemtuzumab, altumomab pentetate, atezolizumab, anetumab, avelumab, bapineuzumab, basiliximab, bectumomab, bermekimab, besilesomab, bevacizumab, bezlotoxumab, brentuximab, brentuximab vedotin, brodalumab, catumaxomab, cemiplimab, cetuximab, cinpanemab, clivatuzumab, crenezumab, tetraxetan, daclizumab, daratumumab, denosumab, dinutuximab, durvalumab, edrecolomab, elotuzumab, emapalumab, enfortumab, enfortumab vedotin, epratuzumab, epratuzumab-SN-38, etaracizumab, gemtuzumab, gemtuzumab ozogamycin, girentuximab, gosuranemab, ibritumomab, inebilizumab infliximab, inotuzumab, inotuzumab ozogamicin, ipilimumab, isatuximab ixekizumab, J591 PSMA-antibody, labetuzumab, lecanemab, mogamulizumab, necitumumab, nimotuzumab, natalizumab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, sacituzumab, sacituzumab govitecan, semorinemab, siltuximab, solanezumab, tacatuzumab, teprotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably atezolizumab, durvalumab, pembrolizumab, rituximab or trastuzumab; or
   the antibody fragment is incorporated into an Fc-fusion protein, which is preferably selected from belatacept, aflibercept, ziv-aflibercept, dulaglutide, rilonacept, romiplostim, abatacept, and alefacept.
27. Modified antibody or modified antibody fragment obtainable by reacting an antibody or antibody fragment, the antibody fragment being optionally incorporated into an Fc-fusion protein, with a compound according to any of items 1 to 22, wherein the antibody or antibody fragment is preferably the same as in item 26.
28. The modified antibody or modified antibody fragment of item 27, which is represented by the following formula (11):

(P—W)ₚ-A (11)
wherein,
P is a group comprising one or more payloads (P¹) as specified in any one of claims 3 to 7, wherein P is preferably a group of formula (2a);
W is F1-RC′, wherein F1 is attached to P and RC′ is a moiety derived from a reactive center (RC) attached to A, F1 and RC being as defined in formulae (4a) and (4b);
A is moiety derived from an antibody or an antibody fragment optionally incorporated into an Fc-fusion protein, said antibody or antibody fragment being as specified in items 25 or 26 above; and
p is an integer of 1 to 5, preferably, p is 1 to 3, more preferably 1 or 2.
29. Modified antibody or modified antibody fragment as defined in item 27 or 28 for use in a method of diagnosing, monitoring, imaging or treating a disease and/or monitoring or imaging a treatment thereof, the method comprising administering the modified antibody or modified antibody fragment to a subject.
30. Method for diagnosing, monitoring, imaging or treating a disease comprising administering the modified antibody or modified antibody fragment according to item 27 or 28 to a subject in need thereof.
31. The modified antibody or modified antibody fragment for use according to item 29, or the method according to item 30, wherein the disease is a neurological disease, a cardiovascular disease, an auto-immune disease or a cancer.
32. The modified antibody or modified antibody fragment for use according to item 29 or 31, or the method according to item 30 or 31, wherein the disease or treatment thereof is selected from the group consisting of Alzheimer's Disease, Amyotrophic Lateral Sclerosis, Cerebral Arteriosclerosis, Encephalopathy, Huntington's Disease, Multiple Sclerosis, Parkinson's Disease, Progressive Multifocal Leukoencephalopathy, Systemic Lupus Erythematosus, systemic sclerosis, Angina including unstable angina, Aortic aneurysm, Atherosclerosis, Cardiac transplant, Cardiotoxicity diagnosis, Coronary artery bypass graft, Heart failure including atrial fibrillation terminated systolic heart failure, hypercholesterolaemia, Ischemia, Myocardial infarction, Thromboembolism, Thrombosis, Ankylosing spondylitis, Autoimmune cytopenias, Autoimmune myocarditis, Crohn's disease, Graft Versus Host disease, Granulomatosis with Polyangiitis, Idiopathic thrombocytopenic purpura, Juvenile arthritis, Juvenile diabetes (Type 1 diabetes), Lupus, Microscopic polyangiitis, Multiple sclerosis, Plaque psoriasis, Psoriasis, Psoriatic arthritis, Rheumatoid arthritis, Ulcerative colitis (UC), Uveitis, and Vasculitis.
33. The modified antibody or modified antibody fragment for use according to item 29 or 31, or the method according to item 30 or 31, wherein the disease is cancer and involves cells selected from lymphoma cells, myeloma cells, renal cancer cells, breast cancer cells, prostate cancer cells, ovarian cancer cells, colorectal cancer cells, gastric cancer cells, squamous cancer cells, lung cancer cells, testicular cancer cells, pancreatic cancer cells, liver cancer cells, melanoma, head-and-neck cancer cells, and any cells growing and dividing at an unregulated and quickened pace to cause cancers; preferably selected from breast cancer cells, lung cancer cells, lymphoma cells, colorectal cancer cells, and head-and-neck cancer cells.

In some other aspects, the present invention includes the following embodiments (“Items”):
1. A compound of formula (1):

V—(Y—P)ₙ (1)
wherein,
V is a peptide comprising a vector that is capable of interacting with the fragment crystallizable (Fc) region of an antibody or fragment thereof, said antibody fragment optionally being incorporated into an Fc-fusion protein;
P is a group comprising one or more payloads P¹;
Y is a reactive moiety capable of reacting with the side chain of an amino acid, preferably lysine, wherein Y is covalently attached to the side chain of an amino acid comprised in V; and
n is an integer from 1 to 3, preferably 1 or 2, and most preferably 1.
2. The compound of item 1, wherein P is P¹, or P is represented by one of the following formulae (2a), (2b) and (2c):

P¹-L-* (2a)

(P¹-L)ₙ′-K—* (2b)

(P¹)ₙ′-K—* (2c)
wherein,
P¹ is a payload;
L is a linker, wherein said linker is optionally cleavable, and wherein preferably the linker comprises one or more atoms selected from carbon, nitrogen, oxygen, phosphorus and sulfur;
K is a branching group that is covalently bonded to group Y and to two or more linkers (L) in formula (2b), or two or more payloads (P¹) in formula (2c), so as to form a dendrimeric structure;
n′ is an integer of from 2 to 8, preferably from 2 to 4, and more preferably 2; and
* indicates covalent attachment to the reactive moiety (Y).
3. The compound of item 1 or 2, wherein P¹ is selected from:
   (i) a moiety derived from
      a chromophore, wherein said chromophore is preferably selected from
         a phosphorophore, and
         a fluorophore such as fluorescein or rhodamine,
      a labelling moiety which may include a radionuclide, wherein said labelling moiety is preferably a moiety containing, or capable of containing, a radionuclide, and more preferably selected from
         a labelling moiety containing, or capable of containing a non-metallic radionuclide such as ¹²⁵I, ¹²³I, ¹³¹I, ¹¹C, ¹⁵O, ¹⁸F, such as a moiety derived from 4-hydroxyphenylpropionate containing a radionuclide such as ¹²⁵I, ¹²³I or ¹³¹I, and
         a chelating agent optionally comprising a chelated radionuclide such as a chelating agent derived from diethylenetriamine pentaacetic acid (DTPA), cyclohexyl diethylenetriamine pentaacetic acid (CH-X-DTPA), desferrioxamine (DFO), N1-(27-amino-11,22-dihydroxy-7,10,18,21-tetraoxo-6,11,17,22-tetraazaheptacosyl)-N1-hydroxy-N4-(5-(N-hydroxyacetamido)pentyl)succinimide (DFO′), N1-(5-(3-(4-aminobutyl)-1-hydroxy-2-oxopiperidine-3-carboxamido)pentyl)-N1-hydroxy-N4-(5-(N-hydroxy-4-((5-(N-hydroxyacetamido)pentyl)amino)-4-oxobutanamido)pentyl)succinimide (DFO-cyclo′), 1-(1,3-carboxypropyl)-4,7-carboxymethyl-1,4,7-tetraacetic acid (NODAGA), 1,4,7,10-tetraazacyclododecane-1-glutaric acid-4,7,10-triacetic acid (DOTAGA), 2,2′-(1,4,7-triazacyclononane-1,4-diyl)diacetate (NO2A), 1,4,7,10-tetraatacyclododecane-1,4,7,10-tetraacetic acid (DOTA), 1,4,7-triazacyclononane-1,4,7-triacetic acid (NOTA), mercaptoacetyl-glycyl-glycyl-glycine (maGGG), mercaptoacetyl-serine-serine-serine (maSSS), 1,4,7,10-tetraatacyclododecane-1,4,7,10-tetraacetic acid-methonine (DOTA-Met), ethylenediaminetetraacetic acid (EDTA), ethylenediaminediacetic acid, triethylenetetraminehexaacetic acid (TTHA), 1,4,8,11-tetraazacyclotetradecane (CYCLAM), 1,4,8,11-tetraazacyclotetradecane-1,4,8,11-tetraacetic acid (TETA), 1,4,8,11-tetraazabicyclo[6.6.2]hexadecane-4,11-diaceticacid (CB-TE2A), 2,2′,2″-(1,4,7,10-tetraazacyclododecane-1,4,7-triyl)triacetamide (DO3AM), 1,4,7,10-tetraazacyclododecane-1,7-diacetic acid (DO2A), 1,5,9-triazacyclododecane (TACD), (3a1s,5a1s)-dodecahydro-3a,5a,8a,10a-tetraazapyrene (cis-glyoxal-cyclam), 1,4,7-triazacyclononane (TACN), 1,4,7,10-tetraazacyclododecane (cyclen), tri(hydroxypyridinone) (THP), 3-(((4,7-bis((hydroxy(hydroxymethyl)phosphoryl)methyl)-1,4,7-triazonan-1-yl)methyl)(hydroxy)phosphoryl)propanoic acid (NOPO), 3,6,9,15-Tetraazabicyclo[9.3.1]pentadeca-1(15),11,13-triene-3,6,9-triacetic acid (PCTA), 2,2′,2″,2′″-(1,4,7,10-tetraazacyclotridecane-1,4,7,10-tetrayl)tetraacetic acid (TRITA), 2,2′,2″,2′″-(1,4,7,10-tetraazacyclotridecane-1,4,7,10-tetrayl)tetraacetamide (TRITAM), 2,2′,2″-(1,4,7,10-tetraazacyclotridecane-1,4,7-triyl)triacetamide (TRITRAM), trans-N-dimethyl-cyclam, 2,2′,2″-(1,4,7-triazacyclononane-1,4,7-triyl)triacetamide (NOTAM), oxocyclam, dioxocyclam, 1,7-dioxa-4,10-diazacyclododecane, cross-bridged-cyclam (CB-cyclam), triazacyclononane phosphinate (TRAP), dipyridoxyl diphosphate (DPDP), meso-tetra-(4-sulfanotophenyl)porphine (TPPS₄), ethylenebishydroxyphenylglycine (EHPG), hexamethylenediaminetetraacetic acid, dimethylphosphinomethane (DMPE), methylenediphosphoric acid, dimercaptosuccinic acid (DMPA), or derivatives thereof;
   (ii) a moiety comprising a conjugation group selected from an optionally substituted conjugated diene, an optionally substituted tetrazine (TZ), an optionally substituted alkyne or azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, an optionally substituted halogenoacetamide, an optionally substituted maleimide, and an optionally substituted or protected thiol, said thiol being preferably protected with a monomethoxytrityl group;
   (iii) a moiety derived from a drug selected from
      an antineoplastic agent such as
         a DNA-alkylating agent e.g. duocarmycin,
         a topoisomerase inhibitor e.g. doxorubicin,
         an RNA-polymerase II inhibitor e.g. alpha-amanitin,
         a DNA cleaving agent e.g. calicheamicin,
         an antimitotic agent or microtubule disruptor e.g. a taxane an auristatin or a maytansinoid,
         an anti-metabolite, such as a derivatives of gemcitabine,
         a Kinesin spindle protein inhibitor such as Filanesib,
         a kinase inhibitor such as ipatasertib or gefitinib,
         nicotinamide phosphoribosyltransferase inhibitor e.g. 2241014-82-2,
         a matrix metallopeptidase 9 inhibitor such as derivatives of CGS27023A,
         a phosphatase inhibitor such as mycrocystin-LR
      an immunomodulatory agent such as fluticasone,
      an anti-infectious disease agent such as rifamycin, clindamycin or reptamulin, and
      radioisotopes, metabolites, pharmaceutically acceptable salts, and/or prodrugs of any of the foregoing,
   if more than one payload (P¹) is present, each P¹ is independently selected from the aforementioned moieties (i) to (iii), the payloads P¹ being preferably identical to each other.
4. The compound of any one of items 1 to 3, wherein P¹ is a chelating agent that optionally comprises a chelated radionuclide, said chelating agent preferably being a moiety derived from DTPA, DOTA, DFO, NOTA, PCTA, CH-X-DTPA, NODAGA, DOTAGA, maSSS, maGGG or DOTA-Methionine, and more preferably a moiety derived from DOTA, DTPA, CH-X-DTPA, PCTA, NOTA or DFO.
5. The compound of item 3 or 4, wherein the radionuclide is selected from ¹²⁴I, ¹³¹I, ⁸⁶Y, ⁹⁰Y, ¹⁷⁷Lu 1 In, ¹⁸⁸Re, ⁵⁵Co, ⁶⁴Cu, ⁶⁷Cu, ⁶⁸Ga, ⁸⁹Zr, ²⁰³Pb, ²¹²Pb, ²¹²Bi, ²¹³Bi, ⁷²As, 211At, ²²⁵Ac, ²²³Ra, ⁹⁷Ru, ¹⁴⁹Tb, ¹⁵²Tb, ¹⁶¹Tb, ⁹⁹mTc, ²²⁶Th, ²²⁷Th, ²⁰¹Tl, ⁸⁹Sr, ⁴/3Sc, ⁴⁷Sc, ¹⁵³Sm, ¹³³Xe, and Al¹⁸F, preferably from ⁸⁹Zr, ¹¹¹In, ⁶⁴Cu, ¹⁷⁷Lu, ⁶⁸Ga, ⁹⁹mTc, ²⁰³Pb, ⁷²As, ⁵⁵Co, ⁹⁷Ru, ²⁰¹Tl, ¹⁵²Tb, ¹³³Xe, ⁸⁶Y, and Al¹⁸F, more preferably from ⁸⁹Zr, ¹¹¹In, ⁶⁴Cu, ¹⁷⁷Lu, ⁶⁸Ga, and ^{99m}Tc, and most preferably from ⁶⁴Cu, ⁹⁹mTc, and ¹¹¹In.
6. The compound of any one of items 1 to 3, wherein P¹ is a moiety derived from exatecan, PNU-159682, amanitin, duocarmycin, auristatin, maytansine, tubulysin, calicheamicin, SN-38, taxol, tubulysin, daunomycin, vinblastine, doxorubicine, methotrexate, pyrrolobenzodiazepine, pyrrole-based kinesin spindle protein (KSP) inhibitors, indolino-benzodiazepine dimers, or radioisotopes and/or pharmaceutically acceptable salts thereof;
   if more than one payload (P¹) is present, each P¹ is independently selected from the aforementioned moieties (i) to (iii) of item 3, the payloads being preferably identical to each other.
7. The compound of any one of items 2 to 6, wherein the linker (L) is selected from
   (a1) an alkylene group having from 1 to 12 carbon atoms, preferably an alkylene group having from 2 to 6 carbon atoms such as a propylene group;
   (b1) a polyalkylene oxide group with 2 or 3 carbon atoms having from 1 to 36 repeating units; preferably a group represented by the formula

—NH—(CH₂CH₂O)ₙ₁—CH₂CH₂—
wherein n1 is an integer of 0 to 35, e.g. 1 to 20;
(c1) a peptidic group comprising 2 to 12 amino acids;
the linker (L) being preferably a group (b1) as defined above; if the compound of formula (1) comprises more than one linker (L), each L is independently selected from the aforementioned groups (a1) to (c1), each linker (L) being preferably a group (b1).
8. The compound of any one of items 2 to 7, wherein the branching group (K) is represented by the formula *—CH(R₁—**)(R₂—**) (3a), or the following formula (3b),

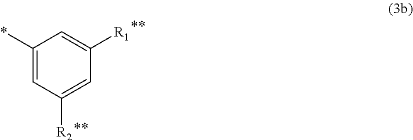

wherein,
R₁ and R₂ are each independently selected from the group consisting of —(CH₂)ₘ₁—** and —(CH₂)ₘ₁R₃—**;
R₃ is selected from the group consisting of —NH—**, —(C═X)R₄—** and —NH(C═X)R₄—**, and is preferably —NH—** or —NH(C═X)R₄—**;
R₄ is —(CH₂)ₘ₂—**, —(CH₂)ₘ₂S—**, —CH(R₇**)₂, —(CH₂)ₘ₂NH—**, or an aryl group of formula (3c):

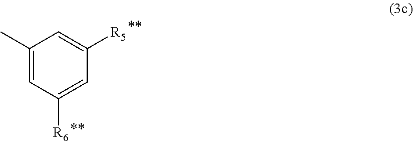

R₅ and R₆ are each independently selected from —(CH₂)ₘ₂—**, —(CH₂)ₘ₂S**—, —CH(CH₂S—**)₂, —(CH₂)ₘ₂NH—**, —CH(R₇—**)₂, —(CH₂)ₘ₂NH(C═X)R₈—**, and —CH₂(CH₂)ₘ₂H—**;
R₇ is —CH₂S—**, (CH₂)ₘ₂—**, or —(CH₂)ₘ₂R₉—*;
R₈ is —(CH₂)ₘ₃S—**;
R₉ is —NH(C═X)ₘ₃S—**;
each X is independently selected from O and S, and is preferably O;
* indicates covalent attachment to the reactive moiety (Y);
** indicates covalent attachment to linker (L) in formula (2b), or to payload (P¹) in formula (2c);
m1, m2 and m3 are each independently selected from 0, 1, 2 and 3, with the proviso that if K is of formula (3a) m1 is not 0; each m1, m2 and m3 being preferably 1.
9. The compound of any one of items 2 to 8, wherein the branching group (K) is represented by one of the following formulae (3d) to (31):

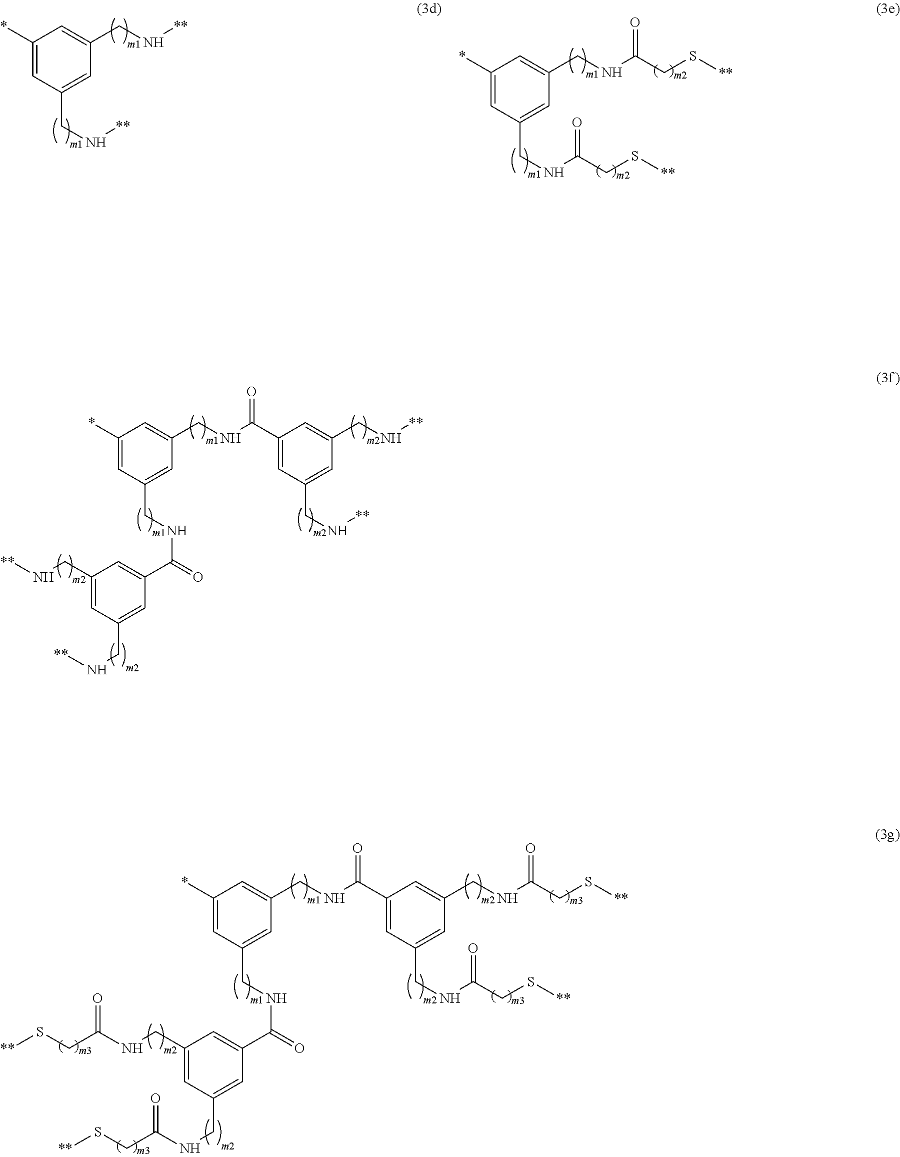

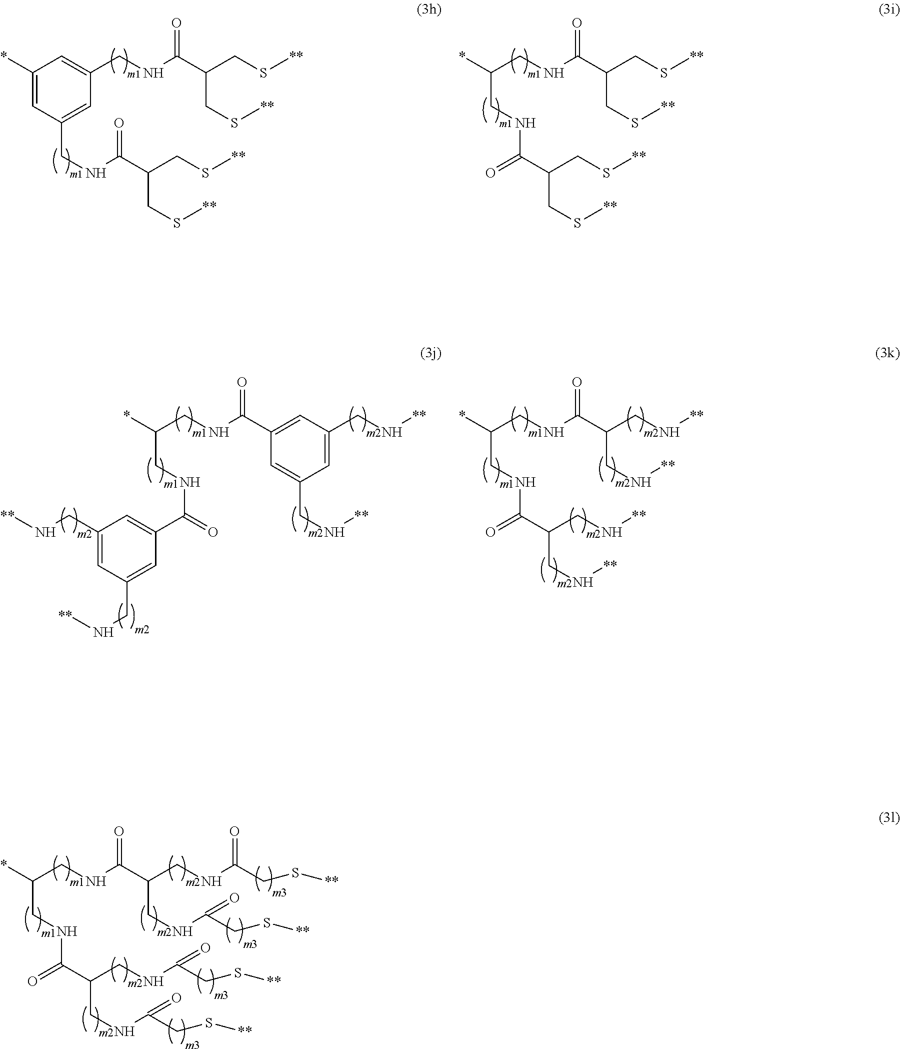

wherein
m1 is 0, 1, 2 or 3, preferably 1;
m2 is 1, 2, 3 or 4, preferably 1;
m3 is 0, 1, 2 or 3, preferably 1;
* indicates covalent attachment to the reactive moiety (Y); and
** indicates covalent attachment to linker (L) or payload (P¹);
the branching group (K) being preferably represented by formula (3d).
10. The compound of any one of items 1 to 9, wherein the reactive moiety (Y) is represented by one of the following formulae (4a) and (4b):

*′-F1-RC-F2-*** (4a)

*′-(F1-RC-F2)-M-*** (4b)
wherein,
RC is a reactive centre, preferably an electrophilic reactive centre, and more preferably a group selected from C═O and C═S;
F1 is a single covalent bond, an atom, or a group of atoms; preferably a CH₂ or NH group of atoms or an atom selected from O and S, or a group of atoms comprising one or more atoms selected from C, N, O, and S; more preferably a CH₂ group of atoms or an atom selected from O and S;
F2 represents an atom, or a group of atoms; preferably an atom selected from O, and S, or a group of atoms comprising one or more atoms selected from C, N, O, and S; more preferably an atom selected from O and S;
M is a group capable of modulating the electron density and stability of F2, preferably a group capable of withdrawing electrons;
*′ indicates covalent attachment to the group (P); and
*** indicates covalent attachment to the peptide (V);
the reactive moiety (Y) being preferably represented by formula (4b).
11. The compound of item 10, wherein the group (M) capable of modulating the electron density and stability of F2 is represented by the following formula (4c):

***‘--M’-B-E--*** (4c)
wherein,
M′ is a moiety derived from succinimide or an aryl group having 6, 10 or 14 ring members and 1, 2 or 3 condensed rings, respectively, or a heteroaryl group having 5 to 20 ring members, 1, 2 or 3 condensed rings and 1 to 4 heteroatoms independently selected from N, O and S, each group being optionally substituted with one or more substituents; preferably a divalent group derived from a phenyl group, a naphthyl group, a pyridyl group, a quinolinyl group, an isoquinolinyl group or a benzotriazolyl group, each group being optionally substituted with one or more substituents and each substituent being preferably selected from —F, —Br, —Cl, —I, —NO₂, —CN, —C₁₋₆-alkyl, —C₁₋₆-alkoxy, —C₁₋₆-amido such as —C(O)NH₂, and combinations thereof such as —CCl₃, —CF₃ or —CH₂NO₂;
B is selected from:
   a single covalent bond, O, S, or a group of atoms NR′, wherein R′ represents a hydrogen atom, —OH, an alkyl group, a cycloalkyl group, a C₂₋₆-alkenylene group, or a C₂₋₆-alkynylene group;
   a group represented by the general formula (4d):

^{♦}—X¹—(CH₂CH₂O)ₙ₂—CH₂CH₂—^{♦♦} (4d)
wherein
X¹ is —C(═O)NH—, —C(═O)—, —NH— or —S—; preferably —C(═O)NH—;
n2 is an integer of 1 to 24, preferably 1 to 10, more preferably 1 to 3;
♦ indicates covalent attachment to M′, and
♦♦ indicates covalent attachment to E;
a peptidic group having 6 to 25 amino acids in the main chain, e.g. 9 amino acids in the main chain, each amino acid being preferably selected from Pro, Gly, Ala, Asn, Asp, Thr, Glu, Gln and Ser; more preferably from Pro, Gly and Ser;
and any combinations thereof;
wherein, B is preferably a single covalent bond or a group having the general formula (4d), more preferably a single covalent bond;
E is C═O, C═S or C(═NR″), wherein R″ represents a hydrogen atom, OH, an alkyl group, a cycloalkyl group, S═O, or S(═O)₂; preferably E is C═O;
   **′ indicates covalent attachment to F2, and
   ** indicates covalent attachment to the peptide (V).
12. The compound of item 10 or 11, wherein the moiety (F1-RC-F2) is represented by one of the following formulae (4a′) to (41′) and/or the group (M) is represented by one of the following formulae (5a) to (5i′):

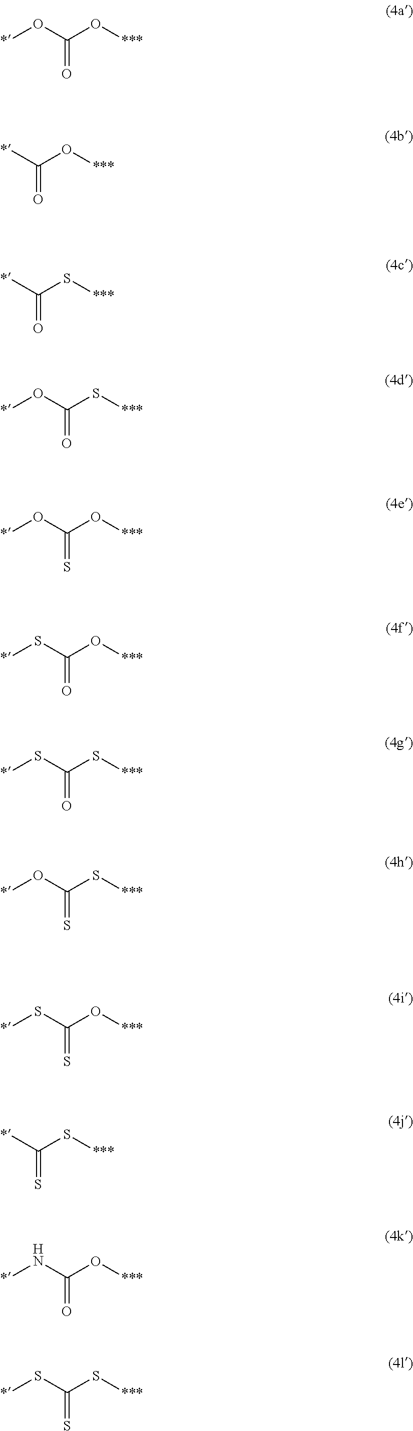

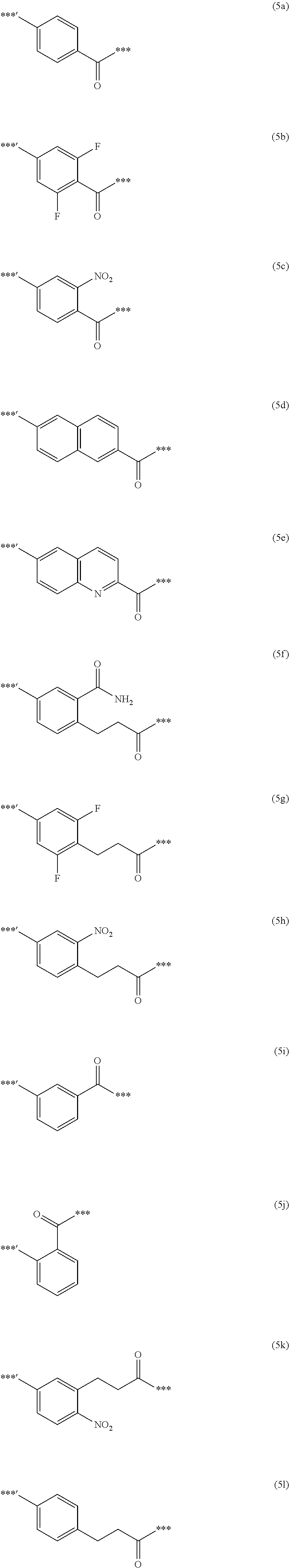

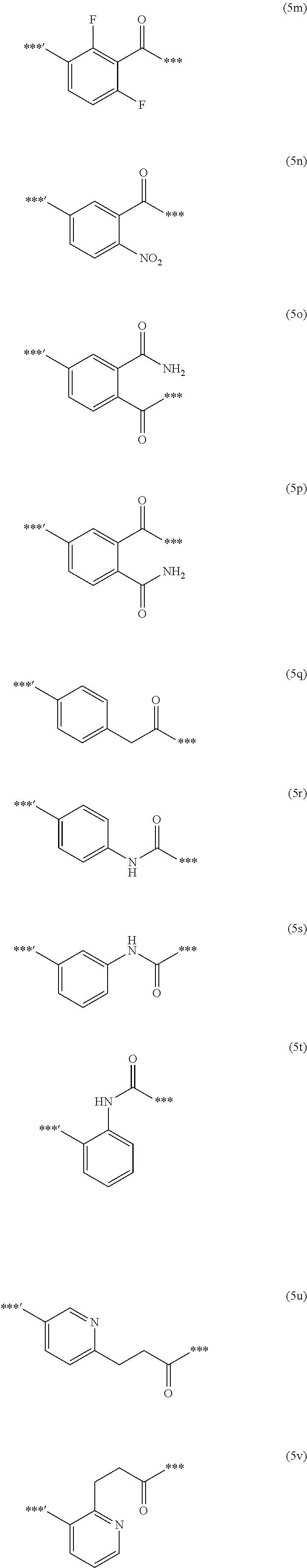

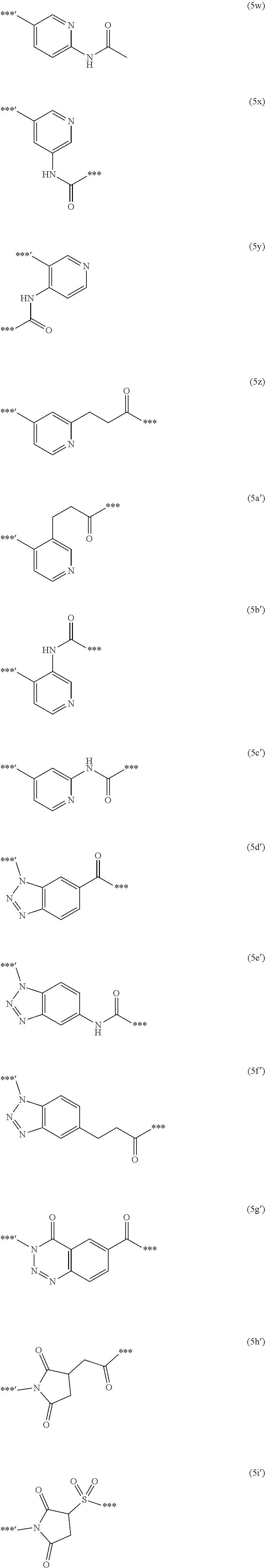

wherein,
*′ indicates covalent attachment to the group (P),
*** indicates covalent attachment to the peptide (V), or to the group (M) if M is present, and
***′ indicates covalent attachment to F2 of the reactive moiety (Y).
13. The compound of any one of items 1 to 12, wherein the reactive moiety (Y) is represented by one of the following formulae (6a) to (6l′):

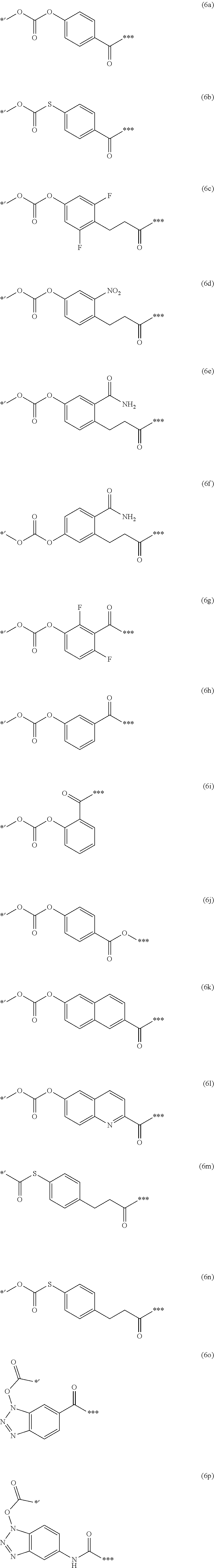

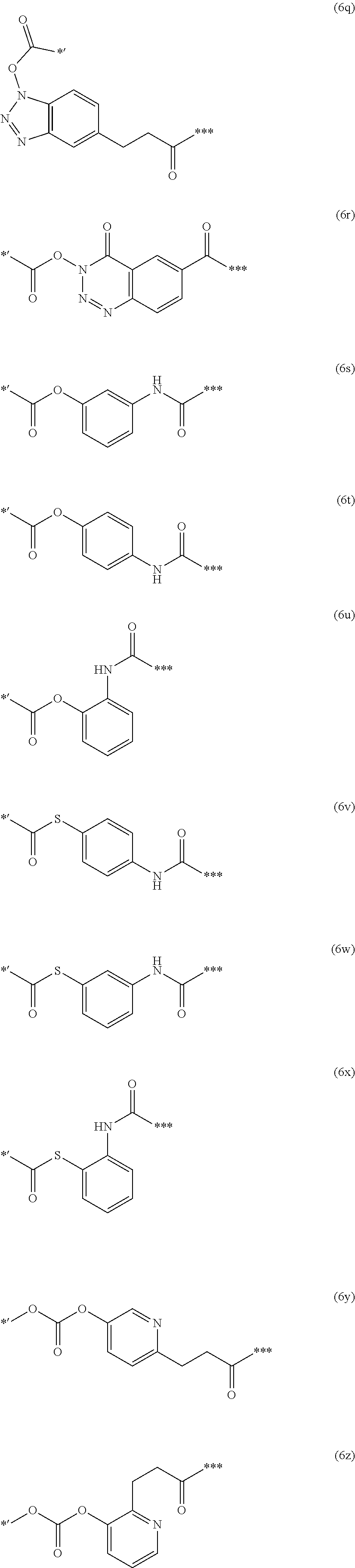

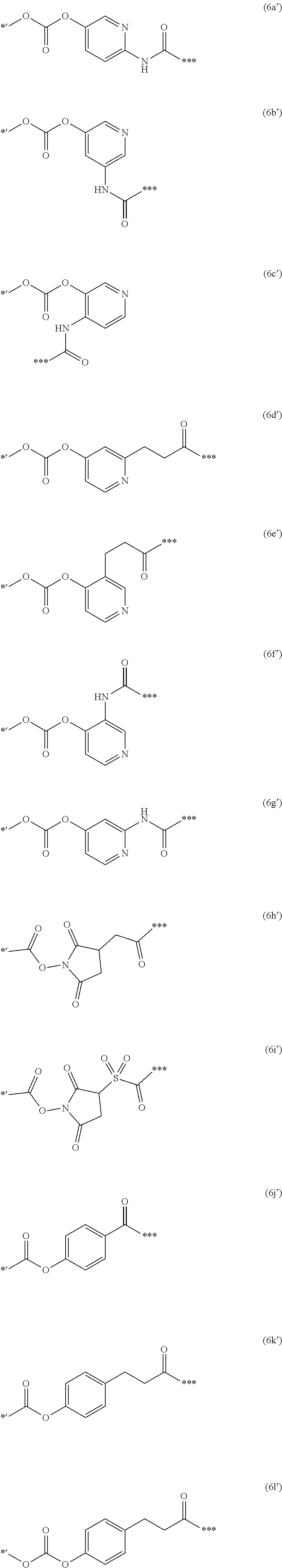

wherein,
*′ indicates covalent attachment to the group (P); and
*** indicates covalent attachment to the peptide (V).
14. The compound of any one of items 1 to 13, wherein the peptide (V) comprises a sequence of 11 to 17 amino acids, e.g. 13 to 17 amino acids, which is preferably cyclic, and more preferably represented by the following formula (7a):

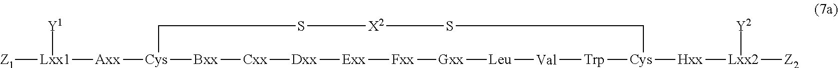

wherein,
   Bxx represents an amino acid selected from an amino acid with an amino group-containing side chain, Ala, Tyr, homo-tyrosine (hTyr), and meta-tyrosine (mTyr); preferably an amino acid selected from Lys, homo-lysine (hLys), ornithine (Orn), 2,3-diaminopropionic acid (Dap), 2,4-diaminobutyric acid (Dab), Ala, Tyr, hTyr, and mTyr; more preferably Ala;
   Cxx, Dxx, Exx, Fxx, Gxx each independently represent an amino acid;
   Axx represents an amino acid, a dicarboxylic acid, or a peptide moiety represented by the following formula (8a):

---Axx1-Axx2-Axx3--- (8a)
wherein, in formula (8a),
Axx1 represents a single covalent bond, or an amino acid such as Arg;
Axx2 represents an amino acid such as Gly or Cys, preferably Gly; and
Axx3 represents an amino acid such Asp or Asn;
Hxx represents an amino acid, or a peptide moiety represented by the following formula (8b):

---Hxx1-Hxx2-Hxx3--- (8b)
wherein, in formula (8b),
Hxx1 represents an amino acid such as Thr;
Hxx2 represents a single covalent bond or an amino acid such as Tyr or Cys; and
Hxx3 represents a single covalent bond, or an amino acid such as His; and the side chain of Axx2 may be covalently attached to the side chain of Hxx2 to form a ring;
wherein preferably both Hxx2 and Hxx3 represent single covalent bonds;
if Axx2 is Cys and Hxx2 is Cys, preferably the side chains of Axx2 and Hxx2 are linked together to form a group of formula —(S—X³—S)—, wherein X³ represents a single covalent bond or a divalent group comprising one or more atoms selected from carbon, nitrogen and oxygen such as a divalent maleimide group, a divalent acetone group or a divalent arylene group; preferably X³ represents a single covalent bond;
Lxx1 and Lxx2 each independently represent a single covalent bond or a trifunctional amino acid such as a diamino-carboxylic acid; with the proviso that at least one of Lxx1 and Lxx2 is a single covalent bond;
Z₁ represents a group selected from:
   a group covalently attached to the N-terminus of Axx, if Lxx1 is a single covalent bond, which is selected from a hydrogen atom, a carbonyl-containing group such as an acetyl group, and a group derived from a compound containing a conjugation group such as biotin, DBCO, TCO, TZ, BCN, an alkyne, an azide, a bromoacetamide, a maleimide and a thiol, wherein said conjugation group is optionally attached via a spacer (S¹);
   a group covalently attached to the N-terminus of Lxx1, if Lxx1 is a trifunctional amino acid and Y¹ is attached to the side chain of Lxx1, which is selected from a hydrogen atom and a carbonyl-containing group such as an acetyl group; and
   a hydrogen atom covalently attached to the side chain of Lxx1, if Lxx1 is a trifunctional amino acid and Y¹ is covalently attached to the N-terminus of Lxx1;
Z₂ represents a group selected from:
   a group covalently attached to the C-terminus of Hxx, if Lxx2 is a single covalent bond, which is selected from —N(H)(R) wherein R represents a hydrogen atom, an alkyl or cycloalkyl group, and a group derived from a compound containing a conjugation group such as biotin, DBCO, TCO, TZ, BCN, an alkyne, an azide, a bromoacetamide, a maleimide or a thiol, wherein said conjugation group is optionally attached via a spacer (S¹);
   a group covalently attached to the C-terminus of Lxx2, if Lxx2 is a trifunctional amino acid and Y² is covalently attached to the side chain of Lxx2; preferably a group represented by N(H)(R) wherein R represents a hydrogen atom, an alkyl group, or a cycloalkyl group; and
   a hydrogen atom covalently attached to the side chain of Lxx2, if Lxx2 is trifunctional amino acid and Y² is covalently attached to the C-terminus of Lxx2;
Y¹ represents a moiety that is only present if Lxx1 is a trifunctional amino acid, wherein Y¹ is covalently attached to the side chain of Lxx1 if Z₁ is attached to the N-terminus of Lxx1, or to the N-terminus of Lxx1 if Z₁ is attached to the side chain of Lxx1;
   wherein Y¹ is derived from a compound containing a conjugation group, which is preferably selected from biotin, DBCO, TCO, TZ, BCN, an alkyne, an azide, a bromoacetamide, a maleimide, and a thiol, and wherein said conjugation group is optionally attached via a spacer (S¹);
Y² represents a moiety that is only present if Lxx2 is a trifunctional amino acid, wherein Y² is covalently attached to the side chain of Lxx2 if Z₂ is attached to the C-terminus of Lxx2, or to the C-terminus of Lxx2 if Z₂ is attached to the side chain of Lxx2;
   wherein Y2 is derived from a compound containing a conjugation group, which is preferably selected from biotin, DBCO, TCO, TZ, BCN, an alkyne, an azide, a bromoacetamide, a maleimide, and a thiol, and wherein said conjugation group is optionally attached via a spacer (S1);
S1 is a spacer represented by the following formula (8c):

α-X⁴—(CH₂CH₂O)ₙ₂—CH₂CH₂—X⁵—β (8c)
wherein
X⁴ is NH, O or S; preferably NH;
X⁵ is NH or C═O, preferably C═O if X⁵ is covalently attached to the peptide (V);
n2 is an integer of 1 to 46, preferably 1 to 24, most preferably 1 to 12; and
α indicates covalent attachment to Y¹ or Y² if X⁵ is covalently attached to the peptide (V), or covalent attachment to the peptide (V) if X⁵ is covalently attached to Y¹ or Y²; and
β indicates covalent attachment to the peptide (V) if X⁴ is covalently attached to Y¹ or Y², or covalent attachment to Y¹ or Y² if X⁴ is covalently attached to the peptide (V);
X² represents a single covalent bond or a divalent group comprising one or more atoms selected from carbon, nitrogen and oxygen such as a divalent maleimide group, a divalent acetone group or a divalent arylene group; preferably a single covalent bond;
wherein at least one of Axx, Bxx, Cxx, Dxx, Exx, Fxx, Gxx and Hxx, preferably one or more of Bxx, Dxx, Exx, Fxx, and Gxx, more preferably one or more of Bxx, Exx, and Gxx, and most preferably Bxx and/or Exx, represents an amino acid with an amino group-containing side chain, Tyr, hTyr, or mTyr, preferably an amino acid with an amino group-containing side chain, more preferably an amino acid selected from Lys, hLys, Orn, Dap, and Dab, which is covalently attached to the reactive moiety (Y) via its side chain; with the proviso that the peptide (V) does not comprise more than three amino acids with an amino group-containing side chain; and
if the reactive moiety (Y) is attached to the peptide (V) via a Tyr, hTyr, or mTyr, the reactive moiety (Y) is preferably a moiety of formula (4a) as defined in item 10.
15. The compound of item 14, wherein at least one of Axx, Cxx, Dxx, Exx, Fxx, Gxx, Hxx, Lxx1 and Lxx2 is defined as follows:
   Axx represents an amino acid selected from Ala, 2,3-diamino-propionic acid (Dap), Asp, Glu, 2-amino suberic acid, α-amino butyric acid, Asn and Gln, a dicarboxylic acid selected from succinic acid, glutaric acid and adipic acid; preferably Ala, Asp or Asn; more preferably Asp; or a peptide moiety of formula (8a), wherein Axx1 is a single covalent bond, Axx2 is Cys, and Axx3 is Asp;
   Cxx represents an amino acid selected from Trp, Phe, Tyr, phenylglycine (Phg), 3-benzothiopen-2-yl-L-alanine, 3-naphthalen-2-yl-L-alanine, 3-biphenyl-4-yl-L-alanine and 3-naphthalen-1-yl-L-alanine; preferably Trp;
   Dxx represents an amino acid selected from His, Ala, 3-pyridin-2-yl-L-alanine, mTyr and Phe; preferably His, Ala or mTyr; more preferably His;
   Exx represents an amino acid selected from Ala, 2-amino-butyric acid (Abu), Gly, Leu, Ile, Val, Met, cyclohexyl alanine (Cha), Phe, Thr, Cys, Tyr, and norleucine (Nle); preferably Ala, Nle or Leu; more preferably Leu;
   Fxx represents an amino acid selected from Ala, Gly, Asn, Ser, Abu, and Asp; preferably Ala or Gly; more preferably Gly;
   Gxx represents an amino acid selected from Ala, Glu, Asp, Gln, His, Arg, Ser, and Asn; preferably Asp or Glu; more preferably Glu;
   Hxx represents an amino acid selected from Thr, Ser, Ala, Asn, Val, Abu, Ile, Met, Leu, Pro, Gln, and Cys; preferably Thr or Ser; more preferably Thr; or a peptide moiety of formula (8b), wherein Hxx1 is Thr, Hxx2 is Cys, and Hxx3 is a single covalent bond;
   Lxx1 and Lxx2 each independently represent an amino acid selected from Dap, Dab, Lys, Orn and hLys, preferably an amino acid selected from Dap, Dab, Lys, Orn and hLys; and
   if Dxx or Exx represents an amino acid with an amino group-containing side chain, Tyr, hTyr, or mTyr, which is covalently attached to the reactive moiety (Y) via its side chain, Gxx is preferably Glu, Gln, His, Arg or Asn, and more preferably Gln.
16. The compound of any of items 1 to 15, wherein the peptide (V) is represented by the following formula (9a):

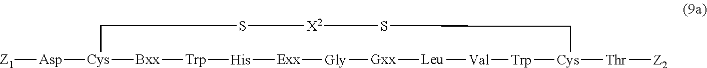

wherein,
   Z₁, Z₂, Bxx, Exx, Gxx and X² are as defined in item 14; and
   at least one of Bxx, Exx and Gxx, preferably Bxx and/or Exx, represents an amino acid with an amino-group containing side chain, Tyr, hTyr, or mTyr; preferably an amino acid with an amino group containing side chain; and more preferably an amino acid selected from Lys, hLys, Orn, Dap, and Dab; said amino acid being covalently bonded to the reactive moiety (Y) via its side chain; and
   if Exx represents an amino acid with an amino group-containing side chain, Tyr, hTyr, or mTyr, preferably an amino acid with an amino group containing side chain; and more preferably an amino acid selected from Lys, hLys, Orn, Dap, and Dab, which is covalently attached to the reactive moiety (Y) via its side chain, Gxx is preferably Glu, Gln, His, Arg or Asn, and more preferably Gln;
and preferably one or two of Bxx, Exx and Gxx is defined as follows:
   Bxx represents an amino acid selected from an amino acid with an amino group-containing side chain, Ala, Tyr, hTyr, and mTyr; preferably Lys, hLys, Orn, Dap, Dab, Ala, Tyr, hTyr, or mTyr; more preferably Ala;
   Exx represents an amino acid selected from Ala, Abu, Gly, Leu, Ile, Val, Met, Cha, Phe, Thr, Cys, Tyr, and Nle; preferably Ala, Nle or Leu; more preferably Leu; and
   Gxx represents an amino acid selected from Ala, Glu, Asp, Gln, His, Arg, Ser, and Asn; preferably Asp or Glu; more preferably Glu.
17. The compound of any one of items 1 to 16, wherein the peptide (V) is represented by one of the following formulae (10a) to (10v), (10b′) and (10g′):

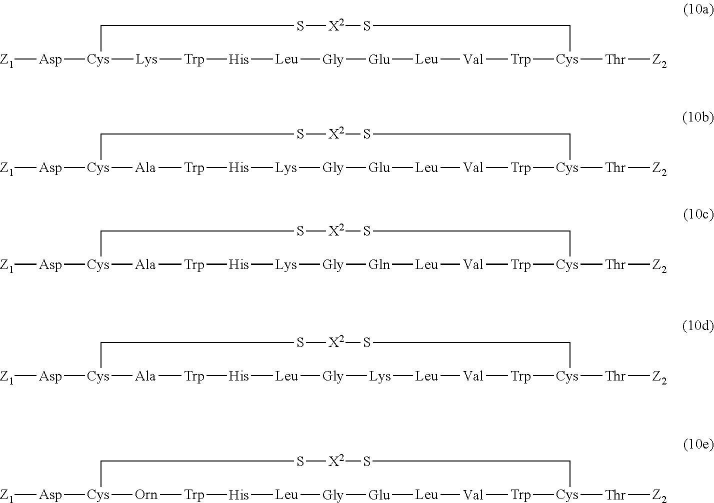

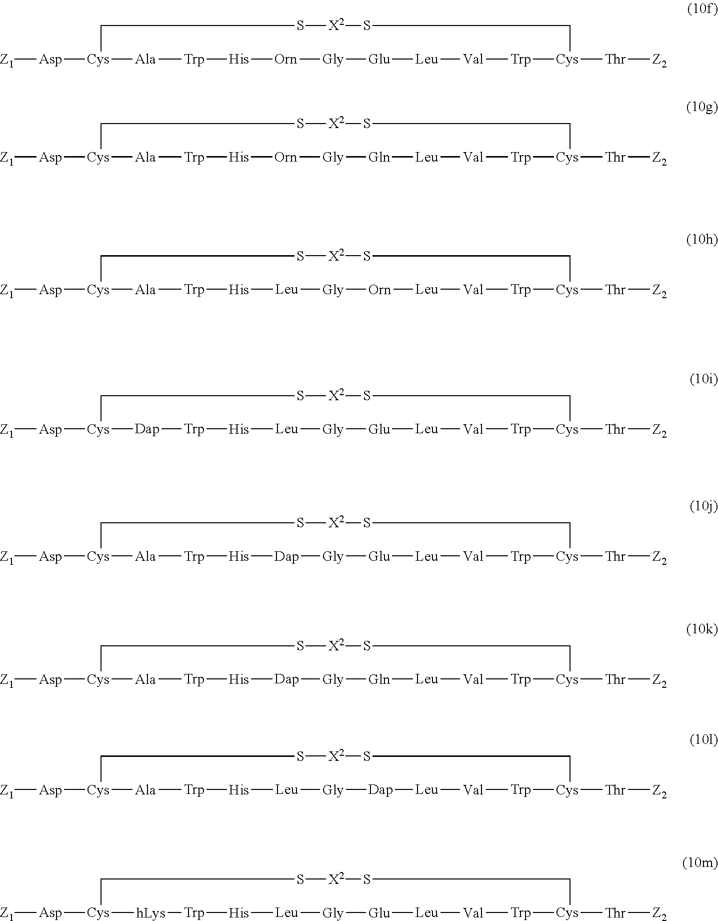

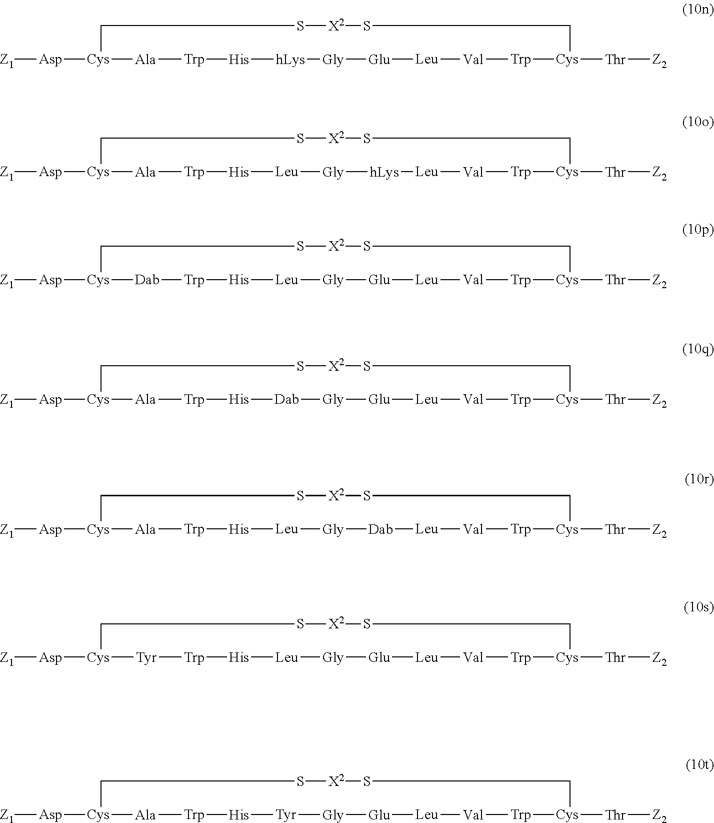

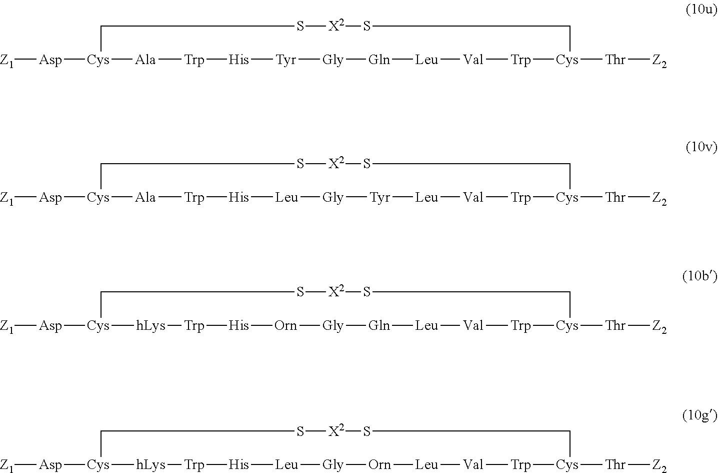

wherein,
Z₁, Z₂, and X² are as defined in item 14; and in the formulae (10a) to (10v), (10b′) and (10g′), the peptide (V) is covalently attached to the reactive moiety (Y) via the side chain of Tyr, Lys, hLys, Orn, Dap, or Dab comprised in V;
the peptide (V) being preferably represented by any one of the formulae (10a), (10b), (10b′) (10c), (10e), (10f), (10g), (10g′) (10h), (10i), (10j), (10k), (10m), (10n), (10p), (10q), (10s), (10t) and (10u), more preferably by any one of the formulae (10e), (10f), (10g), (10h), (10i), (10j), (10k), (10t) and (10u), and even more preferably by any one of the formulae (10e), (10f), (10g), (10h), (10i), (10j), and (10k).
18. The compound of any one of items 1 to 17, which is selected from the group consisting of

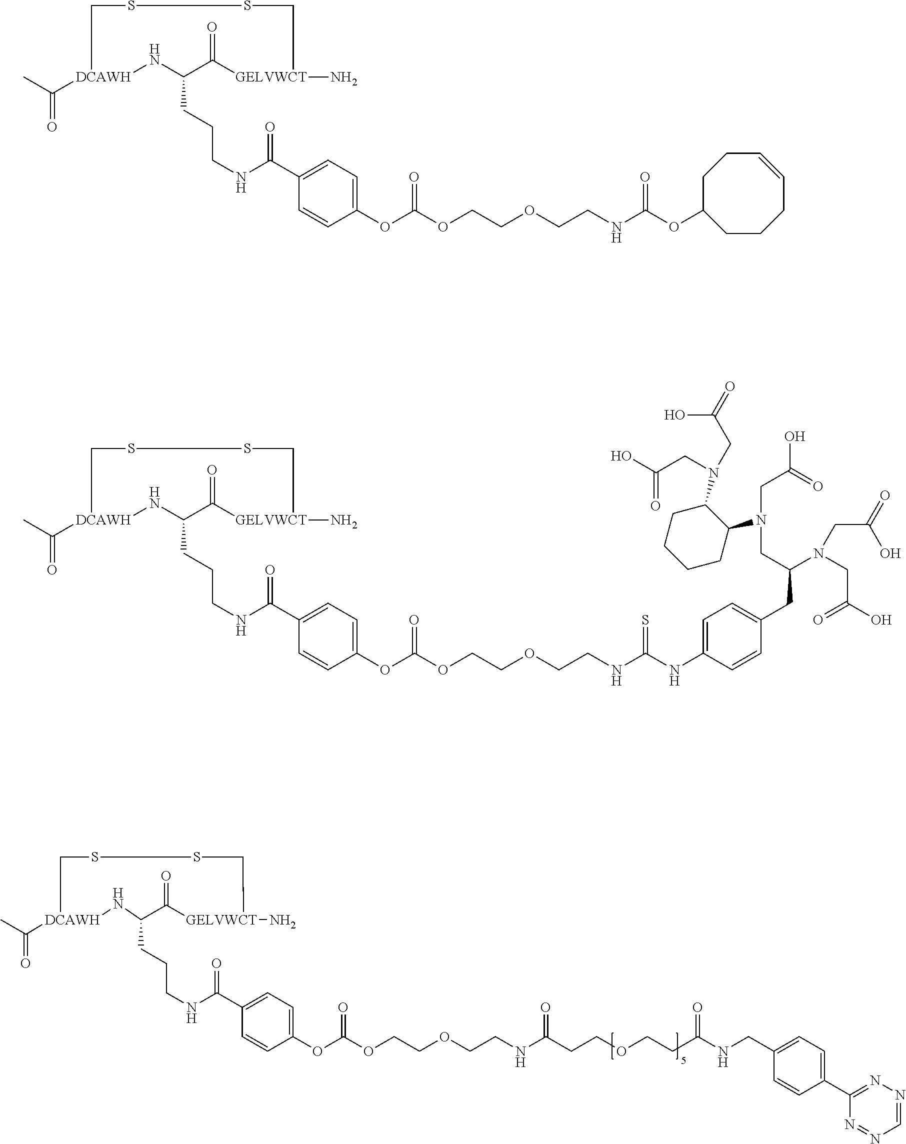

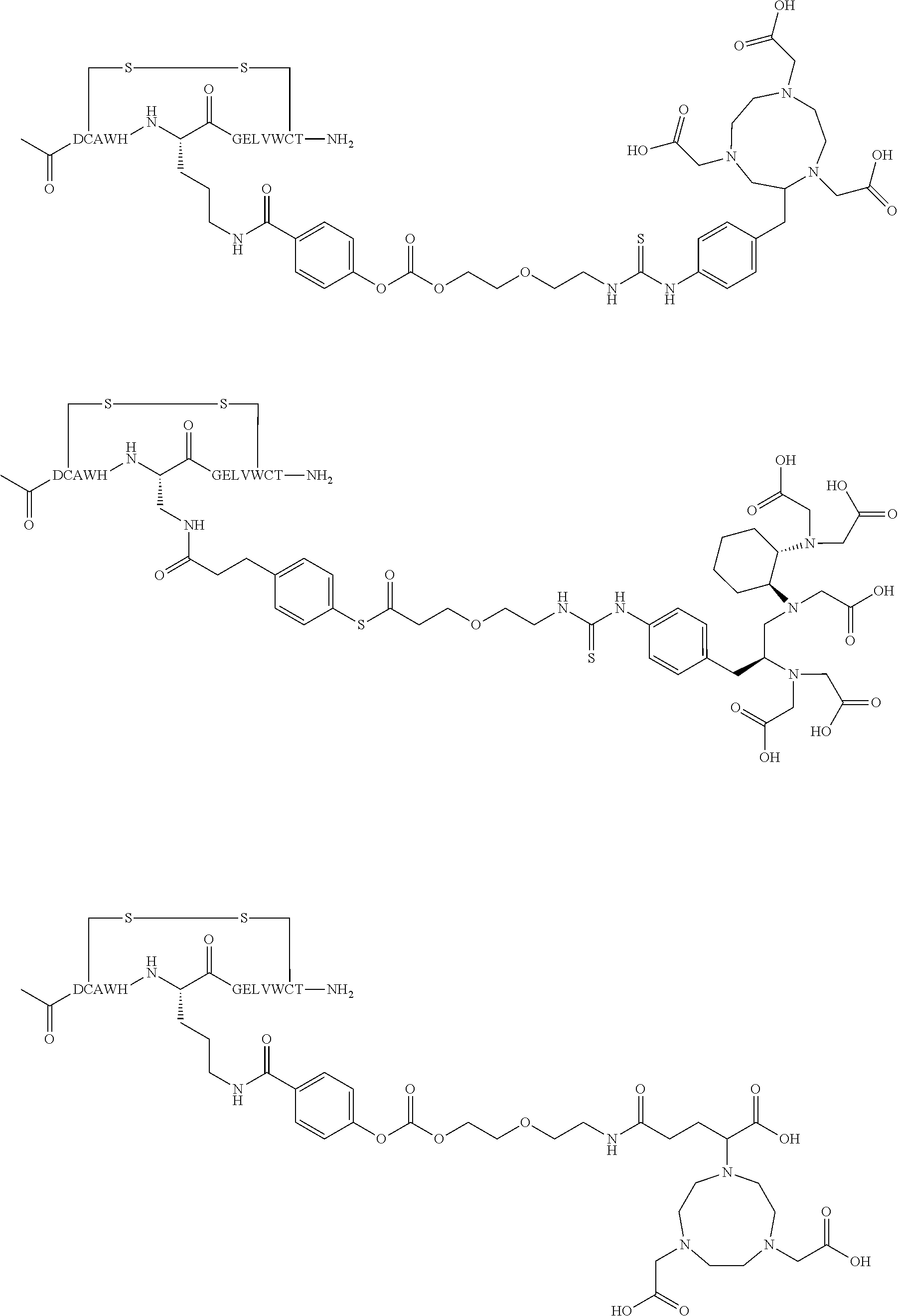

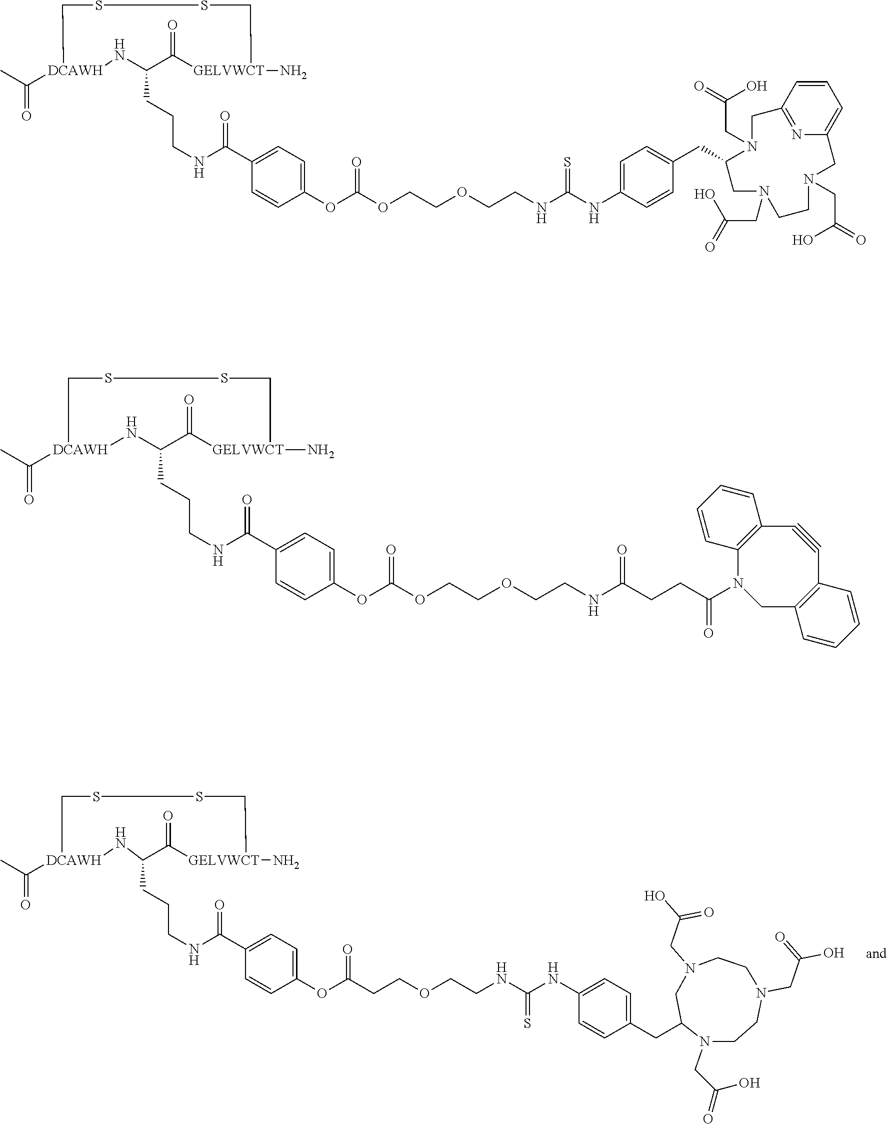

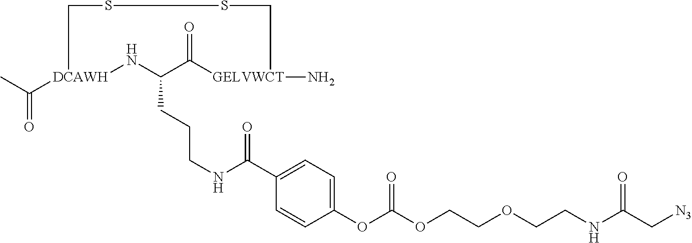

19. Kit for the regioselective modification of an antibody or fragment thereof, the antibody fragment being optionally incorporated into an Fc-fusion protein, comprising the compound of any of items 1 to 18 and a buffer; wherein the buffer has preferably a pH of 5.5 to 11, more preferably 7.0 to 9.5.
20. The kit for the regioselective modification of an antibody or fragment thereof of item 19, wherein the compound is immobilized on a solid phase matrix, preferably wherein the compound is immobilized on the solid support via a biotin-streptavidin interaction, via a covalent linkage obtained by click reaction between an alkyne and azide, via covalent linkage obtained by reaction between a thiol and an acetamide, via covalent linkage obtained by reaction between a derivative of TCO and a derivative of TZ, or via covalent linkage obtained by reaction between a thiol and maleimide.
21. Method for the regioselective modification of an antibody or fragment thereof, the antibody fragment being optionally incorporated into an Fc-fusion protein, comprising reacting an antibody or fragment thereof with a compound according to any of items 1 to 18.
22. The method of item 21, wherein
   the antibody is a monoclonal antibody, preferably an antibody selected from the group consisting of adalimumab, aducanumab, alemtuzumab, altumomab pentetate, atezolizumab, anetumab, avelumab, bapineuzumab, basiliximab, bectumomab, bermekimab, besilesomab, bevacizumab, bezlotoxumab, brentuximab, brentuximab vedotin, brodalumab, catumaxomab, cemiplimab, cetuximab, cinpanemab, clivatuzumab, crenezumab, tetraxetan, daclizumab, daratumumab, denosumab, dinutuximab, durvalumab, edrecolomab, elotuzumab, emapalumab, enfortumab, enfortumab vedotin, epratuzumab, epratuzumab-SN-38, etaracizumab, gemtuzumab, gemtuzumab ozogamycin, girentuximab, gosuranemab, ibritumomab, inebilizumab infliximab, inotuzumab, inotuzumab ozogamicin, ipilimumab, isatuximab ixekizumab, J591 PSMA-antibody, labetuzumab, lecanemab, mogamulizumab, necitumumab, nimotuzumab, natalizumab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, sacituzumab, sacituzumab govitecan, semorinemab, siltuximab, solanezumab, tacatuzumab, teprotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably atezolizumab, durvalumab, pembrolizumab, rituximab or trastuzumab; or
   the antibody fragment is incorporated into an Fc-fusion protein, which is preferably selected from belatacept, aflibercept, ziv-aflibercept, dulaglutide, rilonacept, romiplostim, abatacept, and alefacept.
23. Modified antibody or modified antibody fragment obtainable by reacting an antibody or antibody fragment, the antibody fragment being optionally incorporated into an Fc-fusion protein, with a compound according to any of items 1 to 18, wherein the antibody or antibody fragment is preferably the same as in item 22.
24. Modified antibody or modified antibody fragment as defined in item 23 for use in a method of diagnosing, monitoring, imaging or treating a disease and/or monitoring or imaging a treatment thereof, the method comprising administering the modified antibody or modified antibody fragment to a subject.
25. Method for diagnosing, monitoring, imaging or treating a disease comprising administering the modified antibody or modified antibody fragment according to item 23 to a subject in need thereof.
26. The modified antibody or modified antibody fragment for use according to item 24, or the method according to item 25, wherein the disease is a neurological disease, a cardiovascular disease, an auto-immune disease or a cancer.
27. The modified antibody or modified antibody fragment for use according to item 24 or 26, or the method according to item 25 or 26, wherein the disease or treatment thereof is selected from the group consisting of Alzheimer's Disease, Amyotrophic Lateral Sclerosis, Cerebral Arteriosclerosis, Encephalopathy, Huntington's Disease, Multiple Sclerosis, Parkinson's Disease, Progressive Multifocal Leukoencephalopathy, Systemic Lupus Erythematosus, systemic sclerosis, Angina including unstable angina, Aortic aneurysm, Cardiac transplant, Cardiotoxicity, Coronary artery bypass graft, Heart failure including atrial fibrillation terminated systolic heart failure, hypercholesterolaemia, Ischemia, Myocardial infarction, Thromboembolism, Thrombosis, Ankylosing spondylitis, Autoimmune cytopenias, Autoimmune myocarditis, Crohn's disease, Graft Versus Host disease, Granulomatosis with Polyangiitis, Idiopathic thrombocytopenic purpura, Juvenile arthritis, Juvenile diabetes (Type 1 diabetes), Lupus, Microscopic polyangiitis, Multiple sclerosis, Plaque psoriasis, Psoriasis, Psoriatic arthritis, Rheumatoid arthritis, Ulcerative colitis (UC), Uveitis, and Vasculitis.
28. The modified antibody or modified antibody fragment for use according to item 24 or 26, or the method according to item 25 or 26, wherein the disease is cancer and involves cells selected from lymphoma cells, myeloma cells, renal cancer cells, breast cancer cells, prostate cancer cells, ovarian cancer cells, colorectal cancer cells, gastric cancer cells, squamous cancer cells, lung cancer cells, testicular cancer cells, pancreatic cancer cells, liver cancer cells, melanoma, head-and-neck cancer cells, and any cells growing and dividing at an unregulated and quickened pace to cause cancers; preferably selected from breast cancer cells, lung cancer cells, lymphoma cells, colorectal cancer cells, and head-and-neck cancer cells.

### FIGURES

FIG. 1—Schematic representation of the antibody conjugation approach using the compound of the present invention. A vector capable of interacting with the Fc region of an antibody binds to the Fc region, thereby bringing the reactive moiety in close proximity to the side chain of an amino acid, e.g. a lysine residue, exposed at the surface of the antibody. The reaction between the side chain of the amino acid, e.g. of the lysine residue, and the reactive moiety leads to covalent attachment of the payload (optionally via a linker) to the antibody, and to the concomitant release of the vector.

FIG. 2—Schematic representation of the antibody conjugation approach using the compound of the present invention, which is immobilized on a solid support by click chemistry. A vector capable of interacting with the Fc region of an antibody binds to the Fc region, thereby bringing the reactive moiety in close proximity to the side chain of an amino acid, e.g. a lysine residue, exposed at the surface of the antibody. The reaction between the side chain of the amino acid, e.g. of the lysine residue, and the reactive moiety leads to covalent attachment of the payload (optionally via a linker) to the antibody, and to the concomitant release of the vector-solid support construct.

FIG. 3—Schematic representation of the antibody conjugation approach using the compound of the present invention comprising a branching group and two payload moieties 1 and 2, which are different from each other. A vector capable of interacting with the Fc region of an antibody binds to the Fc region, thereby bringing the reactive moiety in close proximity to the side chain of an amino acid, e.g. a lysine residue, exposed at the surface of the antibody. The reaction between the side chain of the amino acid, e.g. of the lysine residue, and the reactive moiety leads to covalent attachment of the branching group and payloads (optionally via a linker) to the antibody, and to the concomitant release of the vector.

FIG. 4—Schematic representation of the antibody conjugation approach using the compound of the present invention comprising a branching group and two payload moieties (identical to each other). A vector capable of interacting with the Fc region of an antibody binds to the Fc region, thereby bringing the reactive moiety in close proximity to the side chain of an amino acid, e.g. a lysine residue, exposed at the surface of the antibody. The reaction between the side chain of the amino acid, e.g. of the lysine residue, and the reactive moiety leads to covalent attachment of the branching group and payloads (optionally via a linker) to the antibody, and to the concomitant release of the vector.

### DETAILED DESCRIPTION OF THE PRESENT INVENTION

### 1. Definitions

The term “payload” as used herein characterizes a substance (e.g. a naturally occurring or synthetic substance) which can confer a novel functionality when it is attached (conjugated) to an antibody or antibody fragment. In some embodiments, the term “payload” as used herein is to be understood as a labeling moiety (e.g. chromophore, fluorophore, radiolabeled moiety) that enables and/or facilitates the detection and/or visualization of a complementary moiety (e.g. an antibody) to which it is attached. For instance, the labeling moiety can be detected and/or visualized by functional (physiological) imaging techniques known in the art such as computed tomography (CT), positron emission tomography (PET), etc. In some embodiments, the term “payload” as used herein is to be understood as a pharmacologically active substance which can inhibit or prevent the function of cells and/or kill cells. In some embodiments, the term “payload” is to be understood as being synonymous with other terms commonly used in the art such as “cytotoxic agent”, “toxin” or “drug” used in the field of cancer therapy. Alternatively, the payload may be a conjugation group.

The payload may include a group derivable from a functional group that allows covalent attachment of the payload to the remainder of the compound (e.g. to reactive moiety Y in formula (1)) such as a carboxylic acid, a primary amine, a secondary amine, a hydroxyl group, a thiol group, or the like. In some embodiments, the term “payload” as used herein is to be understood as a (solubilizing) moiety which can enhance (improve) the water solubility of the compound to which it is attached. Non-limiting examples of moieties which can enhance water solubility include (a) polyol polymers, such as polymers of glycerol, erythritol, pentaerythritol, or the like, (b) polysaccharides, such as polymers of sucrose, glucose, fructose, sorbitol, or the like, (c) sarcosine polymers (polysarcosines), i.e., polymers of general formula —(N(CH₃)—CH₂—(C═O))ₙ— in which n indicates the number of sarcosine repeating units, (d) moieties comprising one or more solubilizing groups (as described further below), and any combinations or derivatives thereof.

The term “solubilizing group” or “solubilizing moiety” as used herein refers to a hydrophilic group or moiety, which can enhance (improve) the water solubility of the moiety or compound to which it is attached. The solubilizing group can be, for example, a polyalkylene oxide group, such as a polyethylene oxide (PEO) or a polypropylene oxide (PPO) group, or a moiety comprising one or more ionic groups, i.e., functional groups which are charged (anionic or cationic) at physiological pH (7.4), such as moieties derived from amino acids, e.g., from Lys, Glu, Asp, His, Arg, diaminopropionic acid (Dap), diaminobutyric acid (Dab), 2-aminoadipic acid (Aad), Orn. Examples of ionic groups include ammonium groups, guanidinium groups, sulfate groups, phosphate groups, phosphonate groups, and sulfonate groups. In some embodiments, the term “solubilizing moiety” refers to a moiety comprising one or more solubilizing groups. In further embodiments, the solubilizing moiety can consist of one or more solubilizing groups, e.g., amino acids, PEO groups.

The term “polyalkylene oxide” (or “polyalkylene glycol”, “polyoxyalkylene”) as used herein refers to substances of the general structure HO—(X-G)ₙ—H, wherein X represents an akylene group having 2 or 3 carbons atoms, and n indicates the number of repeating units, e.g., 2 to 2000, 4 to 600, 10 to 200, or 15 to 80 repeating units, such as 20 or 40 repeating units, e.g., 16, 20, 24 or 32 PEO repeating units. Thus, the term “polyalkylene oxide group” is to be understood as a divalent group of formula *—O—(X—O)ₙ—**, wherein X and n are as defined above, and * and ** indicate covalent attachment to adjacent moieties. In some embodiments, the term “polyalkylene oxide” can refer to polyethylene oxide (or polyethylene glycol, C₂-polyalkylene oxide), or polypropylene oxide (or polypropylene glycol, C₃-polyalkylene oxide). It is also possible to provide a polyalkylene oxide group, in which two or more different alkylene groups, as defined above, are arranged in a random or block-wise manner.

The term “peptide” as used herein refers to a compound comprising a continuous sequence of at least three amino acids linked to each other via peptide linkages. The term “peptide linkage” in this connection is meant to encompass (backbone) amide bonds as well as modified linkages, which can be obtained if non-natural amino acids are introduced in the peptidic sequence. In this case, the modified linkage replaces the (backbone) amide bond which is formed in the continuous peptide sequence by reacting the amino group and the carboxyl group of two amino acid residues. For instance, the modified linkage may be an ester, a thioester, a carbamide, a thiocarbamide or a triazole linkage. Preferably, the amino acids forming the continuous peptide sequence are linked to each other via backbone amide bonds. The peptide may be linear or branched. In one aspect, the peptide may be cyclic, for instance made of a linear chain of amino acids that has been modified to form a cycle, e.g. “head-to-tail” cyclization, or made of a linear chain of amino acids having side chains covalently attached to each other, e.g. by disulfide bond formation or any other modification. Here, the amino acids include both naturally occurring amino acids as well as non-natural (synthetic) amino acids, as described below.

The term “amino acid” as used herein refers to a compound that contains or is derived from a compound containing at least one amino group and at least one acidic group, preferably a carboxyl group. The distance between amino group and acidic group is not particularly limited. α-, β-, and γ-amino acids are suitable but α-amino acids and especially α-amino carboxylic acids are particularly preferred. The term “amino acid” encompasses both naturally occurring amino acids such as the naturally occurring proteinogenic amino acids, as well as synthetic amino acids that are not found in nature. In the following, a reference to amino acids may be made by means of the 3-letter amino acid code (Arg, Phe, Ala, Cys, Gly, Gln, etc.), or by means of the 1-letter amino acid code (R, F, A, C, G, Q, etc.). Hereinafter, amino acid sequences are written from the N-terminus to the C-terminus (left to right).

The expression “side chain of an amino acid” as used herein may refer to a moiety attached to the α-carbon of an amino acid. For example, the side chain of Ala is methyl, the side chain of Phe is phenylmethyl, the side chain of Cys is thiomethyl, the side chain of Tyr is 4-hydroxyphenylmethyl, etc. Both naturally occurring side chains and non-naturally occurring side chains are included by this definition.

The term “trifunctional” as used herein refers to a compound or moiety having three functional groups that can form or have formed three covalent bonds to adjacent moieties. Thus, the term “trifunctional amino acid” refers to a compound that contains or is derived from a compound containing at least an amino group, an acid group (e.g. a carboxyl group) and another functional group such as an amino group or a carboxyl group.

The term “C-terminal” as used herein refers to the C-terminal end of the amino acid (peptide) chain. Binding to the “C-terminus” means that a covalent bond is formed between the acid group in the main chain (backbone) of the amino acid residue and the binding partner. For instance, binding of group “X” to the C-terminus of amino acid residue “Axx” yields an ester or amide-type structural element *—C(O)—X, wherein the carbonyl group is derived from the acid group of Axx and (*) indicates attachment to main chain.

The term “N-terminal” as used herein refers to the N-terminal end of the amino acid (peptide) chain. Binding to the “N-terminus” means that a covalent bond is formed between the amino group in the main chain (backbone) of the amino acid residue and the binding partner (which replaces one hydrogen atom). For instance, binding of group “X” to the N-terminus of amino acid residue “Axx” yields a structural element X—NH—*, wherein the amino group is derived from Axx and (*) indicates attachment to main chain.

The expression “capable of interacting with the fragment crystallizable (Fc) region of an antibody or fragment thereof” as used herein indicates that the vector can bind to the Fc region of an antibody or antibody fragment as defined hereinbelow. Said interaction/binding can give rise to a targeting effect i.e. to a local increase of the concentration of reactive moiety in proximity to the side chain of an amino acid (e.g. a lysine residue) of the antibody or antibody fragment. The interaction (binding) of a vector with the Fc region of an antibody or antibody fragment can be assessed by using fluorescence polarization (FP) techniques known in the art and described further below. In some aspects, the expression “compound capable of interacting with the Fc region of an antibody or fragment thereof” refers to a compound that retains at least 20%, preferably at least 50%, more preferably at least 80% of the binding affinity of ligand “Fc-III” for the Fc-region of IgG as described by DeLano et al. (Science 2000, 287, 1279-1283) and measured by fluorescence polarization. The compound capable of interacting with the Fc region of an antibody or fragment thereof may have superior bind binding affinity for the Fc region as compared with Fc-III.

The term “antibody” (also synonymously called “immunoglobulin” (Ig)) as used herein covers monoclonal antibodies, polyclonal antibodies, dimers, multimers, multi-specific antibodies (e.g. bispecific antibodies), veneered antibodies, and small immune proteins, provided that it comprises at least one fragment crystallizable (Fc) region. An antibody is a protein generated by the immune system that is capable of recognizing and binding to a specific antigen. A target antigen generally has numerous binding sites, also called epitopes, recognized by complementary-determining regions on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e. a molecule that contains an antigen-binding site that immuno-specifically binds an antigen of a target of interest or part thereof. The antibodies may be IgG e.g. IgG1, IgG2, IgG3, IgG4. Preferably, the antibody is an IgG protein and more preferably an IgG1, IgG2 or IgG4 protein. Most preferably the antibody is an IgG1 protein. The antibody can be human or derived from other species. Preferably the antibody is a human antibody.

The term “monoclonal antibodies” as used herein characterizes antibodies that are identical because they are produced by one type of immune cell and are all clones of a single parent cell.

The term “antibody fragment” as used herein refers to a molecule comprising at least one polypeptide chain derived from an antibody that is not full length and has at least a fragment crystallizable region enabling interaction with a vector.

The term “commercially formulated antibody” as used herein refers to a marketed formulation comprising a therapeutic antibody and one or more excipients. Preferably, the commercially formulated antibody is a formulation marketed in the European Union. Examples of commercially formulated antibodies include Humira®, Lemtrada®, Campath®, Tecentriq®, Bavencio®, Simulect®, LymphoScan®, Xilonix®, Scintimun®, Avastin®, Zinplava®, Blincyto®, Libtayo®, Erbitux®, hPAM4-Cide®, Zenapax®, Darzalex®, Prolia®, Unituxin®, Imfinzi®, Panorex®, Empliciti®, Gamifant®, Rencarex®, Remicade®, Besponsa®, Yervoy®, CEA-Cide®, Poteligeo®, Tysabri®, Portrazza®, Theracim®, Opdivo®, Arzerra®, Lartruvo®, Omnitarg®, Vaxira®, Cyramza®, MabThera®, Rituxan®, Sylvant®, Bexxar®, Herceptin®, Kadcyla®, Stelara®, HuMax-EGFr®, HuMax-CD4®, and biosimilars thereof. Information on commercially formulated antibodies can be found, for instance, in Allgemeine and Spezielle Pharmakologie und Toxicologie, Thomas Karow and Ruth Lang-Roth, Karow, 27^{th} ed. (2018).

Preferably, the commercially formulated antibody is Herceptin® (trastuzumab-containing formulation) as approved for marketing in the European Union by the European Medicines Agency (EMA) under authorization numbers EU/1/00/145/001 and EU/1/00/145/002 (available from Roche), or MabThera® (rituximab-containing formulation) as approved for marketing in the European Union by the EMA under authorization numbers EU/1/98/067/001, EU/1/98/067/002, EU/1/98/067/003 and EU/1/98/067/004.

The term “Fc-fusion protein” as used herein refers to a protein comprising at least an Fc-containing antibody fragment—i.e. an immunoglobulin-derived moiety comprising at least one Fc region—and a moiety derived from a second, non-immunoglobulin protein. The Fc-containing antibody fragment forms part of the Fc-fusion protein and therefore is incorporated into the Fc-fusion protein. The Fc-containing antibody fragment can be derived from an antibody as described hereinabove, in particular from IgG e.g. IgG1, IgG2, IgG3, IgG4. Preferably, the Fc-containing moiety is derived from an IgG1 protein, more preferably from a human IgG1 protein. The non-Ig protein can be a therapeutic protein, for instance a therapeutic protein derived from erythropoietin (EPO), thrombopoietin (THPO) such as THPO-binding peptide, growth hormone, interferon (IFN) such as IFNα, IFNβ or IFNγ, platelet-derived growth factor (PDGF), interleukin (IL) such as IL1α or IL1β, transforming growth factor (TGF) such as TGFα or TGFβ, or tumor necrosis factor (TNF) such as TNFα or TNFβ, or a therapeutic protein derived from a receptor, in particular from a ligand-binding fragment of the extracellular domain of a receptor, for instance derived from cluster of differentiation 2 (CD2), CD4, CD8, CD11, CD14, CD18, CD20, CD22, CD23, CD25, CD33, CD40, CD44, CD52, CD58 (LFA3), CD80, CD86, CD147, CD164, IL2 receptor, IL4 receptor, IL6 receptor, IL12 receptor, epidermal growth factor (EGF) receptor, vascular endothelial growth factor (VEGF) receptor, epithelial cell adhesion molecule (EpCAM), or cytotoxic T-lymphocyte-associated protein 4 (CTLA4). Examples of Fc-fusion proteins include belatacept (Nulojix®), aflibercept (Eyla®), rilonacept (Arcalyst®), romiplostim (NPlate®), abtacept (Orencia®), alefacept (Amevine®), and etanercept (Enbrel®).

The term “reactive moiety” as used herein refers to a moiety that can readily react with a binding partner on another molecule, e.g. with a nucleophile. This is in contrast to moieties that require the addition of catalysts or highly impractical reaction conditions to react (i.e. “non-reactive” or “inert” moieties). Particularly, the expression “reactive moiety” refers to a moiety of a reactive conjugate, which readily reacts with the side chain of Lys of an antibody, preferably trastuzumab (Herceptin® available from Roche) at a molar ratio conjugate to trastuzumab of 2 to 1 when stirred at 1000 rpm in 50 mM NaHCO₃ pH 9.0 at room temperature for 2 hours, leading to the reaction (e.g. attachment of a payload to trastuzumab) of at least 25% of the conjugate, preferably at least 50% of the conjugate, more preferably at least 70% of the conjugate. The attachment of a payload to trastuzumab can be determined by high-resolution mass spectrometry (HRMS) according to the method described in section 9.3.4 below.

The term “chromophore” as used herein refers to an organic or metal-organic compound which is able to absorb electromagnetic radiation in the range of from 350 nm to 1100 nm, or a subrange thereof, e.g. 350-500 nm or 500-850 nm, or 350-850 nm.

The term “phosphorophore” as used herein refers to a compound which, when excited by exposure to a particular wavelength of light, emits light at a different wavelength and lower intensity over a prolonged period of time, e.g. up to several hours.

The term “fluorophore” as used herein refers to a compound which, when excited by exposure to a particular wavelength of light, emits light at a different (higher) wavelength. Fluorophores are usually described in terms of their emission profile or “color”. For example, green fluorophores such as Cy3 or FITC generally emit at wavelengths in the range of 515-540 nm, while red fluorophores such as Cy5 or tetramethylrhodamine generally emit at wavelengths in the range of 590-690 nm. The term “fluorophore” as used herein is to be understood as encompassing, in particular, organic fluorescent dyes such as fluorescein, rhodamine, AMCA, Alexa Fluor dyes (e.g., Alexa Fluor 647), and biological fluorophores.

The term “labelling moiety” (or synonymously “label” or “label group”) as used herein refers to a moiety containing a group which enables and/or facilitates the detection and/or visualization by visual or instrumental means of a complementary moiety (e.g. an antibody) to which it is attached. Examples of labeling moieties include radioactive labels (e.g. radionuclides), contrast agents for magnetic resonance imaging (MRI), and chemicals that absorb or emit light, e.g. chromophores and fluorophores.

The term “chelating agent” as used herein refers to a molecule containing two or more electron donor atoms that can form coordinate bonds to a single central metal ion, e.g. to a radionuclide. Typically, chelating agents coordinate metal ions through oxygen or nitrogen donor atoms, or both. After the first coordinate bond is formed, each successive donor atom that binds creates a ring containing the metal ion. A chelating agent may be bidentate, tridentate, tetradentate, etc., depending on whether it contains 2, 3, 4, or more donor atoms capable of binding to the metal ion.

However, the chelating mechanism is not fully understood and depends on the chelating agent and/or radionuclide. For example, it is believed that DOTA can coordinate a radionuclide via carboxylate and amino groups (donor groups) thus forming complexes having high stability (Dai et al. Nature Com. 2018, 9, 857). The term “chelating agent” is to be understood as including the chelating agent as well as salts thereof. Chelating agents having carboxylic acid groups, e.g. DOTA, TRITA, HETA, HEXA, EDTA, DTPA etc., may, for example, be derivatized to convert one or more carboxylic acid groups to amide groups for attachment to the compound, i.e. to the reactive moiety or the linker, alternatively, for example, said compounds may be derivatized to enable attachment to the compound via one of the CH₂ groups in the chelate ring.

The term “radionuclide” as used herein refers to an atom with an unstable nucleus, which is a nucleus characterized by excess energy available to be imparted either to a newly created radiation particle within the nucleus or to an atomic electron. Radionuclides occur naturally or can be artificially produced. In some embodiments, the radionuclide to be used in the present invention is a medically useful radionuclide including, for example, positively charged ions of radiometals such as Y, In, Cu, Lu, Tc, Re, Co, and Fe. The radionuclide may be selected from ¹²⁴I, ¹³¹I, ⁸⁶Y, ⁹⁰Y, ¹⁷⁷Lu, ¹¹¹In, ¹⁸⁸Re, ⁵⁵Co, ⁶⁴Cu, ⁶⁷Cu, ⁶⁸Ga, ⁸⁹Zr, ²⁰³Pb, ²¹²Pb, ²¹²Bi, ²¹³Bi, ⁷²As, ²¹¹At, ²²⁵Ac, ²²³Ra, ⁹⁷Ru, ¹⁴⁹Tb, ¹⁵²Tb, ¹⁶¹Tb, ⁹⁹mTc, ²²⁶Th, ²²⁷Th, ²⁰¹Tl, ⁸⁹Sr, ^{44/43}Sc, ⁴⁷Sc, ¹⁵³Sm, ¹³³Xe, and Al¹⁸F. Preferably, the radionuclide is selected from ⁸⁹Zr, ¹¹¹In, ⁶⁴Cu, ¹⁷⁷Lu, ⁶⁸Ga, ⁹⁹mTc, ²⁰³Pb, ⁷²As, ⁵⁵Co, ⁹⁷Ru, ²⁰¹Tl, ¹⁵²Tb, ¹³³Xe, ⁸⁶Y, and Al¹⁸F, more preferably from ⁸⁹Zr, ¹¹¹In, ⁶⁴Cu, ¹⁷⁷Lu, ⁶⁸Ga, and ^{99m}Tc, and most preferably from ⁶⁴Cu, ⁹⁹mTc, and ¹¹¹In, in particular ¹¹¹In.

The expression or term “moiety derived from a drug” as used herein refers to a moiety corresponding to a native drug, which differs from the native drug only by the structural modification required for bonding to adjacent moieties, e.g. for bonding to the reactive moiety, linker or branching group comprised in the compound of the present invention. This may include covalent bonds formed by existing functional groups (available in the native drug) or covalent bonds and adjacent functional groups newly introduced for this purpose. By consequence, the drug can be used in its non-modified form (except for the replacement of a hydrogen atom by a covalent bond), or it can be chemically modified in order to incorporate one functional group allowing covalent attachment to the reactive moiety, linker, or branching group comprised in the compound of the present invention. The expression or term “moiety derived from a drug” as used herein is meant to encompass both meanings.

In an analogous manner, the term “derivative” is used to characterize moieties bonded to adjacent moieties, which moieties differ from the molecules from which they are derived only by the structural elements responsible for bonding to adjacent moieties. This may include covalent bonds formed by existing functional groups or covalent bonds and adjacent functional groups newly introduced for this purpose.

The term “native drug” characterizes a compound, for which therapeutic efficacy has been established by in vitro and/or in vivo tests. In a preferred embodiment, the native drug is a compound for which therapeutic efficacy has been established by clinical trials. Most preferably, the native drug is a drug that is already commercially available. The type of therapeutic efficacy to be established and suitable tests to be applied depend of course on the type of medical indication to be treated.

When referring to specific classes of drug molecules, such as an antineoplastic agent, a topoisomerase inhibitor, an RNA-polymerase II inhibitor, a DNA cleaving agent, an antimitotic agent or microtubule disruptor, an anti-metabolite, a kinase inhibitor, an immunomodulatory agent, or an anti-infectious disease agent, these terms are intended to have the meaning generally accepted in the field of medicine, as reflected, for instance, in the Mosby's Medical Dictionary, Mosby, Elsevier 10^{th} ed. (2016), or in Oxford Textbook of Oncology, David J. Kerr, OUP Oxford 3^{rd} ed. (2016).

The term “hydrophobic payload” (or “hydrophobic drug”) as used herein refers to a drug having a calculated CLogP>0, or a hydrophobicity comparable to or greater than monomethyl auristatin E (MMAE), whereby “comparable” means that the hydrophobicity of the drug is within 20% of the hydrophobicity of MMAE. Hydrophobicity can be measured using SlogP, which is defined as the log of the octanol/water partition coefficient (including implicit hydrogens). It can be calculated using the program MOE™ from the Chemical Computing group (see Wildman et al. J Chem Inf Comput Sci. 1999, 39(5), 868-873).

The term “pharmaceutically acceptable salts” as used herein refers to derivatives of disclosed compounds (including the reactive conjugates) wherein the parent compound is modified by making acid or base salts thereof. The pharmaceutically acceptable salts include the non-toxic salts or the quaternary ammonium salts of the parent compound formed, for example, from non-toxic inorganic or organic acids or bases. Lists of suitable salts can be found in Remington's Pharmaceutical Sciences, 17^{th} ed., Mack Publishing Company, Easton, PA, 1985, page 1418, S. M. Berge, L. M. Bighley, and D. C. Monkhouse, “Pharmaceutical Salts,” J. Pharm. Sci. 66 (1), 1-19 (1977); P. H. Stahl and C. G. Wermuth, editors, Handbook of Pharmaceutical Salts: Properties, Selection and Use, Weinheim/Züirich, Wiley-VCH, 2008 and in A. K. Bansal et al., Pharmaceutical Technology, 3(32), 2008. The pharmaceutical salts can be synthesized from the parent compound which contains a basic or acidic moiety by conventional chemical methods. For the reactive conjugates, this can be done before or after incorporating the drug moiety into the compound of the present invention. Unless the context dictates otherwise, all references to compounds (conjugates, modified antibodies, etc.) of the invention are to be understood also as references to pharmaceutically acceptable salts of the respective compounds.

The expression “group capable of modulating the electron density and stability of X” as used herein refers to a group which can modulate (increase or decrease) the properties (electron density/stability) of the neighboring group (X), e.g. moiety (F2) in formulae (4a) and (4b). The modulating group (M) may withdraw or donate electrons to the neighboring group, for instance by an inductive effect and/or a mesomeric effect (see International Union of Pure and Applied Chemistry, Compendium of Chemical Technology, Gold Book 2012, 477-480). Preferably, inductive and mesomeric effects may lead to a displacement of the electronic density distribution towards the modulating group, thereby modulating the electron density and stability of the neighboring group (e.g. F2). The modulation of the electron density can be determined by ¹³C NMR spectroscopy, for instance by measuring the shifts of the carbon atom of the carbonate group and comparing the same with the shift of a reference compound, e.g. compound L6K-carbonate-DOTA described in Example 5 below. A change of the NMR shift of the carbonate signal to higher ppm values (compared to the shift of the reference compound) is indicative of a reduction of the electron density and thus a reduction of stability. A change of the NMR shift of the carbonate signal to lower ppm values (compared to the shift of the reference compound) is indicative of an increase of the electron density and increase of stability. Said modulation of electron density can be used to optimize reactivity and stability of the conjugate of the invention.

According to an embodiment of the present invention, the “group capable of modulating the electron density and stability of X” is selected such that, in the absence of further reagents, the conjugate is stable to degradation (e.g. hydrolysis) which means that the conjugate exhibits less than 50% degradation, preferably less than 25% degradation, more preferably less than 10% degradation, in particular less than 5% degradation, when being mixed with water/DMSO (95/5, v/v) at pH 9 at a concentration of 1 mg/mL and stirred at 500 rpm for 1 hour at 25° C., as determined by HPLC.

The term “electron-withdrawing group” as used herein refers to a group or substituent that can withdraw electrons from the moiety to which it is bonded, i.e. reduce the electron density of this moiety in comparison with the same moiety carrying a hydrogen atom instead of the electron-withdrawing group. Typical electron withdrawing groups include, but are not limited to cyano, nitro, haloalkyl, carboxyl, aryl, sulfonyl, etc. The electron-withdrawing group can exert its electron-withdrawing effect by inductive effect and/or mesomeric effect (as indicated above). The term “electron-withdrawing” as used herein is meant to encompass both meanings. Electron-withdrawing groups/substituents are known in the art and described e.g. by Carey & Sundberg in Advanced Organic Chemistry, Part A: Structure and Mechanisms, 4^{th} Edition.

The term “leaving group” as used herein refers to an atom or group (which may be charged or uncharged) that becomes detached from an atom or a molecule in what is considered to be the residual or main part of the molecule taking part in a specific reaction, for instance a nucleophilic substitution reaction (Pure Appl. Chem. 1994, 66, 1134). Examples of leaving groups include thiophenolates, phenolates, carboxylates, sulfonates.

The term “solid phase matrix” (or synonymously “solid support”, “solid phase” or “solid phase material”) as used herein characterizes a material that is insoluble or can be made insoluble by a subsequent reaction. Representative examples of solid phase material include polymeric or glass beads, microparticles, tubes, sheets, plates, slides, wells, and tapes.

The term “cancer” as used herein means the physiological condition in mammals that is characterized by unregulated cell growth. A tumor comprises one or more cancer cells. Examples of cancer include carcinoma, lymphoma, blastoma, sarcoma, and leukemia or lymphoid malignancies. Further examples of cancer include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, gastrointestinal stromal tumor, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, thyroid cancer and hepatic cancer.

The term “alkyl group” as used herein refers to a linear or branched hydrocarbon group having from 1 to 20 carbon atoms, preferably from 1 to 5 carbon atoms, more preferably a methyl or an ethyl group, or to a cycloalkyl group having from 3 to 20 carbon atoms, preferably from 5 to 8 carbon atoms. The cycloalkyl group may consist of a single ring, but it may also be formed by two or more condensed rings.

The term “aryl” as used herein refers to a radical of a monocyclic or polycyclic (e.g. bicyclic or tricyclic) 4n+2 aromatic ring system (e.g. having 6, 10, or 14 π electrons shared in a cyclic array) having 6-14 ring carbon atoms and zero heteroatoms provided in the aromatic ring system. In some embodiments, an aryl group has 6 ring carbon atoms (e.g. phenyl). In some embodiments, an aryl group has 10 ring carbon atoms (e.g. naphthyl such as 1-naphthyl and 2-naphthyl). In some embodiments, an aryl group has 14 ring carbon atoms (e.g. anthracyl). The term “aryl” as used herein is meant to encompass ring systems, wherein the aryl ring is fused with one or more carbocyclyl or heterocyclyl groups wherein the radical or point of attachment is on the aryl ring (in such instances, the number of carbon atoms designates the number of carbon atoms in the aryl ring system). Unless otherwise specified, the aryl group can be unsubstituted (an “unsubstituted aryl”) or substituted (a “substituted aryl”) with one or more (e.g. 1 to 5) substituents. Non-limiting examples of aryl groups include radicals derived from benzene, naphthalene, anthracene, biphenyl, etc. The term “carbocyclyl” in the context of this disclosure refers to a radical of a non-aromatic cyclic hydrocarbon group having from 3 to 14 ring carbon atoms and zero heteroatoms in the non-aromatic ring system. The term “heterocyclyl” refers to a radical of a 3- to 14-membered non-aromatic ring system having ring carbon atoms and 1 to 4 ring heteroatoms, each heteroatom being independently selected from N, O and S. In heterocyclyl groups that contain one or more nitrogen atoms, the point of attachment can be a carbon atom or nitrogen atom as valency permits.

The term “heteroaryl” as used herein refers to a radical of a 5-14 membered monocyclic or polycyclic (bicyclic, tricyclic) 4n+2 aromatic ring system (e.g. having 6, 10, or 14 π electrons shared in a cyclic array) having ring carbon atoms and 1 to 4 heteroatoms provided in the aromatic ring system, wherein each heteroatom is independently selected from nitrogen, oxygen, and sulfur. In heteroaryl groups that contain one or more nitrogen atoms, the point of attachment can be a carbon atom or a nitrogen atom, as valency permits. Heteroaryl polycyclic ring systems can include one or more heteroatoms in one or both rings. The term “heteroaryl” as used herein is meant to encompass ring systems wherein the heteroaryl ring is fused with one or more aryl, carbocyclyl or heterocyclyl groups wherein the point of attachment is on the heteroaryl ring (in such instances, the number of ring members designates the number of ring members in the heteroaryl ring system). The term “heteroaryl” is also meant to include ring systems, wherein the heteroaryl ring is fused with one or more aryl groups wherein the point of attachment is either on the aryl or heteroaryl ring (in such instances, the number of ring members designates the number of ring members in the fused polycyclic (aryl/heteroaryl) ring system).

The term “substituted aryl group” as used herein means an aryl group in which one or more hydrogen atoms are each independently replaced with a substituent. Non-limiting examples of substituents include —Z, —R, —OR, —SR, —NR₂, —NR₃, —CZ₃, —CN, —OCN, —SCN, —NO₂, —C(O)R, —C(O)NR₂, —SO₃, —S(O)₂R, —C(S)R, —C(O)OR, —C(O)SR, where each Z is independently a halogen (i.e. —F, —Cl, —Br, or —I), and each R is independently —H, —C₁₋₂₀ alkyl, —C₁₋₂₀ alkoxy such as a methoxy group or an ethoxy group, unsubstituted —C₆₋₂₀ aryl, or unsubstituted —C₅₋₁₄ heteroaryl. The heteroaryl group described above may be similarly substituted.

The term “divalent arylene group” refers to a divalent moiety derived from an optionally substituted aryl or heteroaryl group, as defined above, wherein one further hydrogen atom is replaced by covalent bonds allowing attachment to adjacent moieties. A divalent arylene-type disulfide bridge (e.g. a divalent group of formula —S—X²—S—/—S—X³—S— wherein X²/X³ represents a divalent arylene group) can be obtained by side-chain-to-side-chain cyclization according to techniques known in the art (see Stefanucci et al. in ACS Med. Chem. Lett. 2017, 8, 449-454, and Beard et al. Bioorg. & Med. Chem. 2018, 26, 3039-3045).

The term “divalent xylene group” as used herein refers to a divalent moiety derived from one of the three isomers of dimethylbenzene (i.e. ortho-xylene, meta-xylene, para-xylene), in which one hydrogen atom of each methyl group is replaced by a covalent bond allowing attachment to an adjacent moiety. Preferably, the divalent xylene group is a divalent meta-xylene group. A divalent xylene-type disulfide bridge (e.g. a divalent group of formula —S—X²—S—/—S—X³—S— wherein X²/X³ represents a divalent xylene group) can be obtained by side-chain-to-side-chain cyclization in the presence of e.g. dibromo-xylene as described by Stefanucci et al. in ACS Med.Chem. Lett. 2017, 8, 449-454.

The term “divalent maleimide group” as used herein refers to a divalent moiety derived from maleimide, in which the hydrogen atoms at positions 2 and 3 are each replaced by a covalent bond allowing attachment to an adjacent moiety. A divalent maleimide-type disulfide bridge (e.g. a divalent group of formula —S—X²—S—/—S—X³—S— wherein X²/X³ represents a divalent maleimide group) can be obtained by side-chain-to-side-chain cyclization in the presence of e.g. 2,3-dibromomaleimide or another suitable reagent as described by Kuan et al. in Chem. Eur. J. 2016, 22, 17112-17129.

The term “divalent acetone group” as used herein refers to a divalent moiety derived from acetone (ACE), in which one hydrogen atom of each methyl group is replaced by a covalent bond allowing attachment to an adjacent moiety. A divalent ACE-type disulfide bridge (e.g. a divalent group of formula —S—X²—S—/—S—X³—S— wherein X²/X³ represents a divalent ACE group) can be obtained by side-chain-to-side-chain cyclization in the presence of e.g. dibromoacetone or dichlororoacetone (see e.g. Assem et al. Angew. Chem. Int. Ed. Engl. 2015, 54(30), 8665-8668).

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful.

Unless specified otherwise or the context dictates otherwise, references to groups being “substituted” or “optionally substituted” are to be understood as references to the presence (or optional presence, as the case may be) of at least one substituent selected from F, Cl. Br, I, CN, NO₂, NH₂, NH—C₁₋₆-alkyl, N(C₁₋₆-alkyl)₂, —X—C₁₋₆-alkyl, —X—C₂₋₆-alkenyl, —X—C₂₋₆-alkynyl, —X—C₆₋₁₄-aryl, —X-(5-14-membered heteroalkyl with 1-3 heteroatoms selected from N, O, S), wherein X represents a single bond, —(CH₂)—, —O—, —S—, —S(O)—, —S(O)₂—, —NH—, —CO—, or any combination thereof including, for instance, —C(O)—NH—, —NH—C(O)—. The number of substituents is not particularly limited and may range from 1 to the maximum number of valences that can be saturated with substituents. It is typically 1, 2 or 3 and usually 1 or 2, most typically 1.

Unless specified otherwise, all valencies of the individual atoms of the compounds or moieties described herein are saturated. In particular, they are saturated by the indicated binding partners. If no binding partner or a too small number of binding partners is indicated, the remaining valencies of the respective atom are saturated by a corresponding number of hydrogen atoms.

Unless specified otherwise, chiral compounds and moieties may be present in the form of a pure stereoisomer or in the form of a mixture of stereoisomers, including the 50:50 racemate. In the context of the present invention, references to specific stereoisomers are to be understood as references to compounds or moieties, wherein the designated stereoisomer is present in at least 90% enantiomeric excess (ee), more preferably at least 95% ee and most preferably 100% ee, wherein % ee is defined as (|R−S|)/(R+S)*100% with R and S representing the amount of moles of the respective enantiomers.

Unless specified otherwise or dictated otherwise by the context, all connections between adjacent amino acid groups are formed by peptide (amide) bonds.

Unless the context dictates otherwise, and/or alternative meanings are explicitly provided herein, all terms are intended to have meanings generally accepted in the art, as reflected by IUPAC Gold Book (status of 1 Aug. 2020), or the Dictionary of Chemistry, Oxford, 6^{th} Ed.

### 2. Overview

The present invention is based on the surprising finding that the regioselective attachment of payloads to an antibody or antibody fragment can be accomplished using the compound of the present invention, and more particularly that the said regioselective attachment can be accomplished in one single step, e.g. without need for further chemical reaction to cleave a covalent bond between the vector and the antibody or antibody fragment.

An efficient conjugation process was achieved by introducing a reactive group capable of remaining stable upon storage and synthesis, but able to form a covalent bond with the side chain of an amino acid at the surface of the antibody or antibody fragment, while simultaneously cleaving another covalent bond to release the vector.

The resulting modified antibody or modified antibody fragment, e.g. ADC or antibody-radionuclide conjugate, can be used in a method of diagnosing, monitoring, imaging or treating disease, in particular cancer.

### 3. Compound of Formula (1)

The present invention relates to a compound represented by the general formula (1):

V—(Y—P)ₙ (1)

The compound of formula (1) contains a peptide comprising a vector capable of interacting with (having binding affinity for) the Fc region of an antibody or fragment thereof, said antibody fragment being optionally incorporated into an Fc-fusion protein, one, two or three (n=1, 2 or 3) reactive moieties capable of reacting with the side chain of an amino acid, each reactive moiety being attached to the side chain of an amino acid comprised in the peptide, and the groups (P) attached thereto, which may each comprise one or more payloads P¹.

In one preferred embodiment, the compound contains one or two (n=1 or 2) reactive moieties capable of reacting with the side chain of an amino acid, each reactive moiety being attached to the side chain of an amino acid comprised in the peptide, and one or two (n=1 or 2) groups (P) attached thereto, which may each comprise one or more payloads P¹. Most preferably, the compound contains one (n=1) reactive moiety capable of reacting with the side chain of an amino acid, the reactive moiety being attached to the side chain of an amino acid comprised in the peptide, and one (n=1) group (P) which comprises one or more payloads P¹.

### 3.1 Group (P)

The group P is a group comprising one or more payloads P¹. This group is not particularly limited and any group comprising a payload such as a labelling and/or pharmaceutically active molecule can be employed as long as it can be covalently attached to the reactive moiety Y.

In one embodiment, P is a payload P¹ as described in section 3.2 below.

In another embodiment, the attachment of the group (P) to the reactive moiety may be made via a linking group (or “linker”) and/or via a branching group. In the context of this disclosure, the linking group and/or branching group may be considered as being part of the group (P). Accordingly, in an embodiment, the group (P) is represented by one of the following formulae (2a), (2b) and (2c):

P¹-L-* (2a)

(P¹-L)ₙ′-K—* (2b)

(P¹)ₙ′-K—* (2c)
wherein,
P¹ is a payload as described further below, e.g. a chelating agent optionally comprising a chelated radionuclide such as ¹⁷⁷Lu-DOTA, or a moiety derived from a drug;
L is a linker;
n′ is an integer of from 2 to 8, preferably 2 to 4, and more preferably 2;
K is a branching group that is covalently bonded to the reactive moiety (Y) and to two or more linkers (L) in formula (2b), or two or more payloads (P¹) in formula (2c), so as to form a dendrimeric structure; and
* indicates covalent attachment to the reactive moiety (Y).

In one preferred embodiment, P is P¹ or a group represented by formula (2a) or (2c). More preferably, P is P¹ or a group represented by formula (2a), and most preferably by formula (2a).

Accordingly, the compound of the present invention may be represented by one of the following formulae (2a′) to (2d′):

V—(Y—P¹)ₙ (2a′)

V—(Y-L-P¹)ₙ (2b′)

V—(Y—K-(L-P¹)ₙ′)ₙ (2c′)

V—(Y—K—(P¹)ₙ′)ₙ (2d′)
wherein,
V, Y, L, K, n and n′ are as described above, and
P¹ is a payload as described further below.

In a preferred embodiment, the compound of the present invention is represented by formula (2a′) or (2b′). Most preferably, the compound the present invention is represented by formula (2b′).

The linker comprised in the group of formulae (2a) and (2b) is a divalent group, preferably comprising one or more atoms selected from carbon, nitrogen, oxygen, phosphorus and sulfur.

In one embodiment, the linker can be selected from
(a1) an alkylene group having from 1 to 12 carbon atoms, preferably an alkylene group having from 2 to 6 carbon atoms such as an ethylene group or a propylene group;
(b1) a polyalkylene oxide group with 2 or 3 carbon atoms having from 1 to 36 repeating units optionally bonded to one or both of the adjacent moieties via an amino group or an additional alkylene group; preferably a group represented by the formula —NH—(CH₂CH₂O)ₙ₁—CH₂CH₂— wherein n1 is an integer of 0 to 35, e.g. 1 to 20; and
(c1) a peptidic group comprising 2 to 12 amino acids.

The linker may be a cleavable or non-cleavable linker. In an embodiment, the linker is a non-cleavable linker. In another embodiment, the linker is a cleavable linker, for instance a cleavable peptidic group (c1).

The cleavable linker may be a linker capable of specifically releasing the payload upon internalization in a target cell. It may utilize an inherent property of the target cell, e.g. a tumor cell, for selectively releasing the payload from the modified antibody or modified antibody fragment, namely (1) protease-sensitivity (enzyme-triggered release linker system), (2) pH-sensitivity, (3) glutathione-sensitivity, or (4) glucoronidase sensitivity. In a specific embodiment, the linker is a cleavable linker comprising a valine-citrulline (Val-Cit) or valine-alanine (Val-Ala) dipeptide that can serve as a substrate for intracellular cleavage by Cathepsin B (Cat B).

In another specific embodiment, the linker is a cleavable linker comprising a self-immolative moiety capable of releasing the payload by elimination- or cyclization-based mechanism. An example of a cleavable linker comprising a self-immolative moiety is the para-amino benzyloxycarbonyl (PABC) linker as used e.g. in the bremtuximab-vedotin conjugate Adcetris® (Younes et al. N. Engl. J. Med. 2010, 363, 1812-1821; Jain et al. Pharm. Res. 2015, 32(11), 3526-3540). The PABC-containing linker comprises a protease-sensitive Val-Cit-PABC dipeptide linker unit, which can be recognized and cleaved by Cat B. The linker unit can be attached to the reactive moiety (and after antibody modification to the antibody) by means of a maleimidocaproyl moiety. Such a linker can help avoid steric conflicts in substrate recognition by Cat B. After enzymatic cleavage of the citrulline-PABC amide bond, the resulting PABC-substituted payload spontaneously undergoes a 1,6-elimination that releases the free payload as the product into the target cell. Accordingly, the group according to formula (2a) may represent vedotin, i.e. a group consisting of a payload moiety derived from monomethyl auristatin E attached to the reactive moiety via a linker comprising a Val-Cit-PABC unit.

In another specific embodiment, the linker is a cleavable linker comprising a C-terminal dipeptide unit capable of acting as a highly specific substrate for the exopeptidase activity of Cat B (exo-Cat B). Examples of exo-Cat B-cleavable linkers systems are described in WO 2019/096867 A1. In particular, the linker can comprise a C-terminal dipeptide unit (“Axx-Ayy” or “Ayy-Axx”) as defined in claim 1, 2 or 3 of WO 2019/096867 A1.

The branching group is a multivalent group, e.g. a trivalent or tetravalent group, which is covalently attached to the reactive moiety as well as to two or more payloads or linkers thereby forming a branched (tree-like) dendrimeric structure. The branching group may be a group derived from a core molecule containing a functional group that allows covalent attachment to the reactive moiety and, additionally, two or more branches, preferably two branches, wherein each branch contains a functional group that allows covalent attachment of further moieties, e.g. payloads or linkers. In one embodiment, each branch may be sub-branched in two or more further branches, wherein each terminal branch contains a functional group that allows covalent attachment of further moieties, e.g. payloads or linkers. Each successive repeating unit along all the branches starting from the core molecule forms a “branching generation” until the terminating generation results. For example, if the core molecule is covalently attached to the reactive moiety and contains two branches, each being bonded to a further moiety, there is one repeating unit along all branches starting from the core molecule and the branching group is of Generation 1 (G1) (the core molecule being designated as Generation 0). If each branch of Generation 1 is sub-branched in two branches, each being bonded to a further moiety, there are two repeating units along all branches starting from the core molecule and each of the two branching groups attached to the Generation 1 branch is of Generation 2 (G2), and so on. The branching group as used herein is preferably of G1 or G2 generation.

In one embodiment, the branching group is represented by one of the following formulae (3a) and (3b):

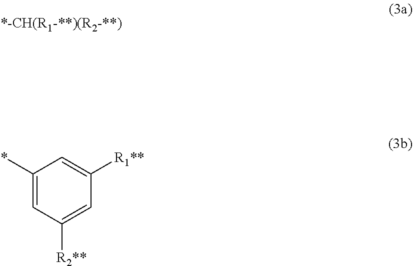

wherein,
R₁ and R₂ are each independently selected from the group consisting of —(CH₂)ₘ₁—** and —(CH₂)ₘ₁R₃—**;
R₃ is selected from the group consisting of —NH—**, —(C═X)R₄—** and —NH(C═X)R₄—**, and is preferably —NH—** or —NH(C═X)R₄—**;
R₄ is —(CH₂)ₘ₂—**, —(CH₂)ₘ₂S—**, —CH(R₇—**)₂, —(CH₂)ₘ₂NH—**, or an aryl group of formula (3c):

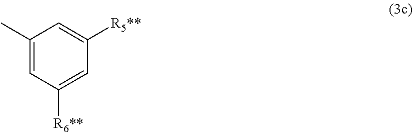

R₅ and R₆ are each independently selected from —(CH₂)ₘ₂—**, —(CH₂)ₘ₂S**—, —CH(CH₂S—**)₂, —(CH₂)ₘ₂NH—**, —CH(R₇—**)₂, —(CH₂)ₘ₂NH(C═X)R₈—**, and —CH₂(CH₂)ₘ₂H—**;
R₇ is —CH₂S—**, —(CH₂)ₘ₂—**, or —(CH₂)ₘ₂R₉**;
R₈ is —(CH₂)ₘ₃S—**;
R₉ is —NH(C═X)ₘ₃S—**;
each X is independently selected from O and S, and is preferably O;
* indicates covalent attachment to the reactive moiety (Y), optionally via a linker (L2);
** indicates covalent attachment to linker (L) in formula (2b), or to payload (P¹) in formula (2c);
m1, m2 and m3 are each independently selected from 0, 1, 2 and 3, with the proviso that if K is of formula (3a) m1 is not 0; each m1, m2 and m3 being preferably 1.

In one embodiment, the branching group is represented by one of the following formulae (3d) to (31):

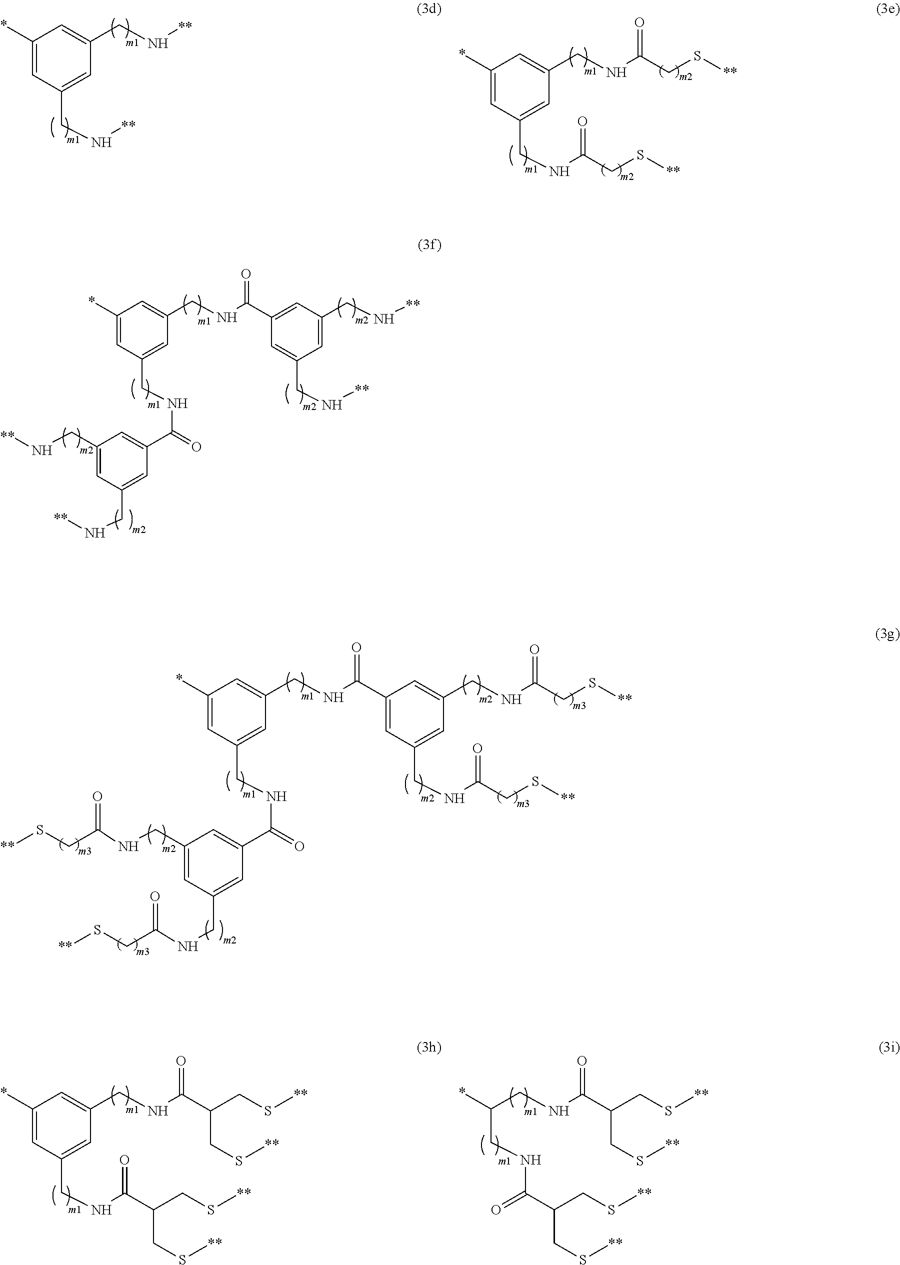

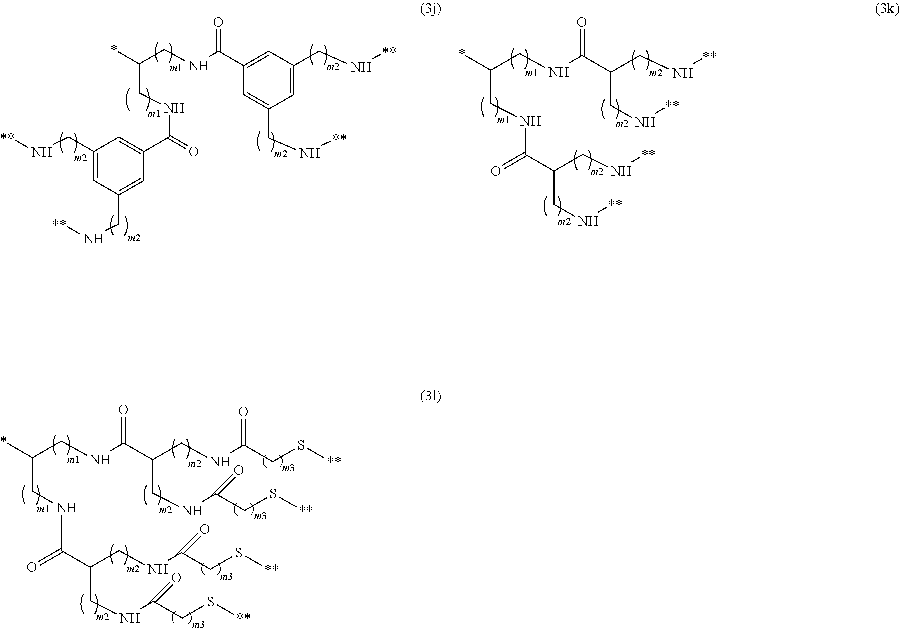

wherein,
m1 is 0, 1, 2 or 3, preferably 1;
m2 is 1, 2, 3 or 4, preferably 1;
m3 is 0, 1, 2 or 3, preferably 1;
* indicates covalent attachment to the reactive moiety, and
** indicates covalent attachment to the linker or payload.

In a preferred embodiment, the branching group is represented by formula (3d) or formula (3e). Accordingly, if a branching group is present, the compound of the present invention may be represented by formulae (2c′) or (2d′) in which K is a branching group of formula (3d) or formula (3e). More preferably, the compound of the present invention is represented by formula (2d′) in which K is a branching group of formula (3d) or (3e), in particular a branching group of formula (3d).

If the branching group (K) is attached to the reactive moiety via a linker (L²), the linker is a divalent group, preferably a divalent group comprising one or more atoms selected from carbon, nitrogen, oxygen, phosphorus and sulfur, and more preferably a divalent group selected from
(a2) an alkylene group having from 1 to 12 carbon atoms, preferably an alkylene group having from 2 to 6 carbon atoms such as an ethylene group or a propylene group; and
(b2) a polyalkylene oxide group with 2 or 3 carbon atoms having from 1 to 6 repeating units optionally bonded to one or both of the adjacent moieties via an amino group or an additional alkylene group; preferably a group represented by the formula η-(C═X)—NH—(CH₂CH₂O)ₙ₃—CH₂CH₂—θ, wherein n3 is an integer of 0 to 10, e.g. 1 to 4, X is selected from O and S, η indicates covalent attachment to K and 6 indicates covalent attachment to Y.

In one embodiment, the branching group (K) is a moiety derived from a compound comprising one or more trifunctional amino acids, such as Lys, Orn, Dab, or Dap, wherein the one or more trifunctional amino acids provide functional groups allowing covalent attachment to the reactive moiety (Y) and at least two branches, wherein each branch contains a functional group allowing covalent attachment to further moieties, e.g., payloads, linkers, or amino acids. For example, the branching group may be a peptidic moiety comprising two or more trifunctional amino acids, e.g. 2, 3 or 4 trifunctional amino acids, in which the peptide N-terminus is covalently attached to a reactive moiety (Y), the peptide C-terminus is covalently attached to a payload (P¹) (as described further below) or a chain terminating group (G) (as described further below), such as NH₂, and the side chain of each trifunctional amino acid is covalently attached to a payload (P¹) in formula (2c) or to a linker (L) in formula (2b).

If the peptidic moiety contains three or more amino acid residues, these residues may be arranged in a linear fashion, wherein each amino acid is bonded to no more than two adjacent amino acids. Alternatively, they may form a branched, e.g., dendrimeric, structure, wherein at least one amino acid is bonded to a side-chain of another amino acid, such that there is at least one amino acid that is bonded to three or more amino acids.

In one embodiment, the branching group (K) is represented by the following formula (3m):

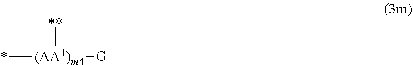

In formula (3m), m4 represents an integer of 1 to 10, preferably 1 to 6, more preferably 1, 2 or 3, * indicates covalent attachment to the reactive moiety (Y) in formula (1), and ** indicates covalent attachment to a linker (L) in formula (2b) or to a payload (P¹) in formula (2c).

Each AA1 is independently a moiety derived from a trifunctional amino acid, such as a diamino-carboxylic acid, e.g., Orn, Lys, Dab, or Dap. Preferably, each AA1 is independently selected from Orn, Lys, Dab and Dap, more preferably from Orn and Lys, even more preferably Lys. The side chain of the trifunctional amino acid is attached to a linker (L) in formula (2b) or to a payload (P¹) in formula (2c).

In another aspect, AA¹ can comprise further linkers and/or amino acids in addition to the trifunctional amino acid mentioned above. Such further linkers and/or amino acids can, for example, be selected from a polyethylene oxide group having from 1 to 20, e.g., 1 to 10, repeating units and/or one or more amino acids (which do not contribute to branching), e.g., 2, 3 or 4 amino acids, wherein preferably each amino acid is independently selected from Arg, Cit, homo-Phe (hPHe) and Phe. In one embodiment, AA1 comprises, in addition to the trifunctional amino acid mentioned above, a peptidic linker comprising 2 to 12 amino acids, which is optionally cleavable, preferably a cleavable peptidic linker comprising a Val-Cit unit, a Val-Ala unit, a Val-Cit-PABC, or a Val-Cit-PABC-DMEA unit.

G is absent, or represents one selected from:
a hydrogen atom,
a group of formula —N(H)(R), wherein R is selected from a hydrogen atom, an alkyl group and a cycloalkyl group; and
a group of formula —N(H)(R)-(CH₂)ₙ₁₃—(C═O)N(H)(R), wherein each R is independently selected from a hydrogen atom, an alkyl group and a cycloalkyl group, and n13 is an integer of 1 to 6, preferably 1 or 2;

If G is absent, the resulting free valence forms a covalent bond to a linker (L) in formula (2b) or to a payload (P¹) in formula (2c);

with the proviso that if m4 is 1, G is absent.

In a preferred embodiment, the branching group (K) is represented by the following formula (3n):

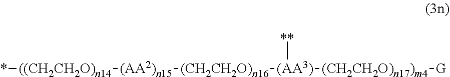

In formula (3n), *, ** m4 and G are as defined in formula (3m).

Each AA² represents an amino acid, preferably an amino acid independently selected from Arg, Cit, hPhe and Phe.

AA³ represents a trifunctional amino acid, which is preferably selected from Dap, Dab, Orn and Lys, more preferably from Orn and Lys. The side chain of the trifunctional amino acid is attached to a linker (L) in formula (2b) or to a payload (P¹) in formula (2c).

Each n14 is independently an integer of 0 to 10, preferably 1 to 8, more preferably 5; each n15 is an integer of 0 to 5, preferably 0 to 3, more preferably 0 or 1; each n16 and each n17 is independently an integer of 0 to 10, preferably 0 to 5, more preferably 0 or 1.

In a more preferred embodiment, the branching group (K) is represented by the following formula (3o):

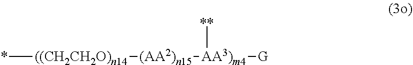

In formula (3o), *, ** m4 and G are as defined in formula (3m).

Each AA² represents an amino acid, preferably an amino acid independently selected from Arg, Cit, hPhe and Phe.

AA³ represents a trifunctional amino acid, which is preferably selected from Dap, Dab, Orn and Lys, more preferably from Orn and Lys, even more preferably Lys. The side chain of the trifunctional amino acid is attached to a linker (L) in formula (2b), or to a payload (P¹) in formula (2c).

Each n14 is independently an integer of 0 to 10, preferably 1 to 8, more preferably 5; and each n15 is an integer of 0 to 5, preferably 0 to 3, more preferably 0 or 1.

### 3.2 Payload (P¹)

The payload (P¹) to be used is not particularly limited and any payload such as a labelling and/or pharmaceutically active molecule can be employed.

According to one embodiment, the payload is selected from:
(i) a moiety derived from
   a chromophore, wherein said chromophore is preferably selected from
      a phosphorophore, and
      a fluorophore such as fluorescein or rhodamine,
   a labelling moiety which may include a radionuclide, wherein said labelling moiety is preferably a moiety containing, or capable of containing, a radionuclide, and more preferably selected from
      a labelling moiety containing, or capable of containing a non-metallic radionuclide such as ¹²⁵I, ¹²³I, ¹³¹I, ¹¹C, ¹⁵O, ¹⁸F, such as a moiety derived from 4-hydroxyphenylpropionate containing a radionuclide such as ¹²⁵I, ¹²³I or ¹³¹I, and
         a chelating agent optionally comprising a chelated radionuclide such as a chelating agent derived from diethylenetriamine pentaacetic acid (DTPA), cyclohexyl diethylenetriamine pentaacetic acid (CH-X-DTPA), desferrioxamine (DFO), N1-(27-amino-11,22-dihydroxy-7,10,18,21-tetraoxo-6,11,17,22-tetraazaheptacosyl)-N1-hydroxy-N4-(5-(N-hydroxyacetamido)pentyl)succinimide (DFO′), N1-(5-(3-(4-aminobutyl)-1-hydroxy-2-oxopiperidine-3-carboxamido)pentyl)-N1-hydroxy-N4-(5-(N-hydroxy-4-((5-(N-hydroxyacetamido)pentyl)amino)-4-oxobutanamido)pentyl)succinimide (DFO-cyclo′), 1-(1,3-carboxypropyl)-4,7-carboxymethyl-1,4,7-tetraacetic acid (NODAGA), 1,4,7,10-tetraazacyclododecane-1-glutaric acid-4,7,10-triacetic acid (DOTAGA), 2,2′-(1,4,7-triazacyclononane-1,4-diyl)diacetate (NO2A), 1,4,7,10-tetraatacyclododecane-1,4,7,10-tetraacetic acid (DOTA), mercaptoacetyl-glycyl-glycyl-glycine (maGGG), mercaptoacetyl-serine-serine-serine (maSSS), 1,4,7,10-tetraatacyclododecane-1,4,7,10-tetraacetic acid-methonine (DOTA-Met), 1,4,7-triazacyclononane-1,4,7-triacetic acid (NOTA), ethylenediaminetetraacetic acid (EDTA), ethylenediaminediacetic acid, triethylenetetraminehexaacetic acid (TTHA), 1,4,8,11-tetraazacyclotetradecane (CYCLAM), 1,4,8,11-tetraazacyclotetradecane-1,4,8,11-tetraacetic acid (TETA), 1,4,8,11-tetraazabicyclo[6.6.2]hexadecane-4,11-diaceticacid (CB-TE2A), 2,2′,2″-(1,4,7,10-tetraazacyclododecane-1,4,7-triyl)triacetamide (DO3AM), 1,4,7,10-tetraazacyclododecane-1,7-diacetic acid (DO2A), 1,5,9-triazacyclododecane (TACD), (3a1s,5a1s)-dodecahydro-3a,5a,8a,10a-tetraazapyrene (cis-glyoxal-cyclam), 1,4,7-triazacyclononane (TACN), 1,4,7,10-tetraazacyclododecane (cyclen), tri(hydroxypyridinone) (THP), 3-(((4,7-bis((hydroxy(hydroxymethyl)phosphoryl)methyl)-1,4,7-triazonan-1-yl)methyl)(hydroxy)phosphoryl)propanoic acid (NOPO), 3,6,9,15-Tetraazabicyclo[9.3.1]pentadeca-1 (15),11,13-triene-3,6,9-triacetic acid (PCTA), 2,2′,2″,2′″-(1,4,7,10-tetraazacyclotridecane-1,4,7,10-tetrayl)tetraacetic acid (TRITA), 2,2′,2″,2′″-(1,4,7,10-tetraazacyclotridecane-1,4,7,10-tetrayl)tetraacetamide (TRITAM), 2,2′,2″-(1,4,7,10-tetraazacyclotridecane-1,4,7-triyl)triacetamide (TRITRAM), trans-N-dimethyl-cyclam, 2,2′,2″-(1,4,7-triazacyclononane-1,4,7-triyl)triacetamide (NOTAM), oxocyclam, dioxocyclam, 1,7-dioxa-4,10-diazacyclododecane, cross-bridged-cyclam (CB-cyclam), triazacyclononane phosphinate (TRAP), dipyridoxyl diphosphate (DPDP), meso-tetra-(4-sulfanotophenyl)porphine (TPPS4), ethylenebishydroxyphenylglycine (EHPG), hexamethylenediaminetetraacetic acid, dimethylphosphinomethane (DMPE), methylenediphosphoric acid, dimercaptosuccinic acid (DMPA), 1,4,7,10-tetraaza-1,4,7,10-tetra(2-carbamoylmethyl)cyclododecane (TCMC), or derivatives thereof;
(ii) a moiety comprising a conjugation group selected from an optionally substituted conjugated diene, an optionally substituted tetrazine (TZ), an optionally substituted alkyne or azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, an optionally substituted halogenoacetamide, an optionally substituted maleimide, and an optionally substituted or protected thiol, said thiol being preferably protected with a monomethoxytrityl group;
(iii) a moiety derived from a drug selected from
   an antineoplastic agent such as
      a DNA-alkylating agent, e.g. duocarmycin,
      a topoisomerase inhibitor, e.g. doxorubicin,
      an RNA-polymerase II inhibitor, e.g. alpha-amanitin,
      a DNA cleaving agent, e.g. calicheamicin,
      an antimitotic agent or microtubule disruptor, e.g. a taxane, an auristatin or a maytansinoid,
      an anti-metabolite, such as a derivatives of gemcitabine,
      a Kinesin spindle protein inhibitor such as Filanesib,
      a kinase inhibitor such as ipatasertib or gefitinib,
      nicotinamide phosphoribosyltransferase inhibitor, e.g. 2241014-82-2,
      a matrix metallopeptidase 9 inhibitor such as derivatives of CGS27023A, or
      a phosphatase inhibitor such as mycrocystin-LR,
   an immunomodulatory agent such as fluticasone,
   an anti-infectious disease agent such as rifamycin, clindamycin or reptamulin, and
   radioisotopes, metabolites, pharmaceutically acceptable salts, and/or prodrugs of any of the foregoing; and
(iv) a moiety comprising one or more solubilizing groups, e.g., 2, 3, 4, or 5 solubilizing groups, each solubilizing group being preferably independently selected from moieties comprising one or more ionic groups and polyalkylene oxide groups; wherein the moiety preferably comprises one or more C₂₋₃ polyalkylene oxide groups, and wherein preferably each C₂₋₃ polyalkylene oxide group independently comprises from 4 to 600, preferably from 10 to 200, more preferably from 15 to 80 repeating units.

If the compound of formula (1) contains more than one payload, each P¹ can be independently selected from the aforementioned moieties (i) to (iii) or (i) to (iv). Preferably, the payloads are identical to each other.

In one embodiment, the payload is a chelating agent that optionally comprises a chelated radionuclide, said chelating agent preferably being a moiety derived from DTPA, DOTA, DFO, NOTA, PCTA, CH-X-DTPA, NODAGA, DOTAGA, maSSS, maGGG or DOTA-Methionine, and more preferably a moiety derived from DOTA, DTPA, CH-X-DTPA, PCTA, NOTA or DFO.

In one embodiment, the radionuclide is selected from ¹²⁴I, ¹³¹I, ⁸⁶Y, ⁹⁰Y, ¹⁷⁷Lu, ¹¹¹In, ¹⁸⁸Re, ⁵⁵Co, ⁶⁴Cu, ⁶⁷Cu, ⁶⁸Ga, ⁸⁹Zr, ²⁰³Pb, ²¹²Pb, ²¹²Bi, ²¹³Bi, ⁷²As, ²¹¹At, ²²⁵Ac, ²²³Ra, ⁹⁷Ru ¹⁴⁹Tb, ¹⁵²Tb ¹⁶¹Tb, ⁹⁹mTc ²²⁶Th, ²²⁷Th, ²⁰¹Tl, ⁸⁹Sr, ^{44/43}Sc, ⁴⁷Sc, ¹⁵³Sm, ¹³³Xe, and Al¹⁸F, preferably from ⁸⁹Zr, ¹¹¹In, ⁶⁴Cu, ¹⁷⁷Lu, ⁶⁸Ga, ⁹⁹mTc, ²⁰³Pb, ⁷²As, ⁵⁵Co, ⁹⁷Ru, ²⁰¹Tl, ¹⁵²Tb, ¹³³Xe, ⁸⁶Y, and Al¹⁸F, more preferably from ⁸⁹Zr, ¹¹¹In, ⁶⁴Cu, ¹⁷⁷Lu, ⁶⁸Ga, and ^{99m}Tc, and most preferably from ⁸⁹Zr, ⁶⁴Cu, ^{99m}Tc, and ¹¹¹In.

In one preferred embodiment, the payload is selected from:
a moiety (i) derived from DOTA, PCTA, DTPA or CH-X-DTPA that chelates ¹¹¹In, most preferably CH-X-DTPA that chelates ¹¹¹In;
a moiety (i) derived from NOTA, NODAGA or PCTA that chelates ⁶⁴Cu, most preferably NOTA that chelates ⁶⁴Cu;
a moiety (i) derived from DOTA, DFO, DFO′ or DFO-cyclo′ that chelates ⁸⁹Zr, most preferably DFO that chelates ⁸⁹Zr.

In one embodiment, the payload is (ii) a moiety selected from a moiety comprising a conjugation group (as listed above) to allow later attachment of a further payload as specified under items (i) and (iii) herein. This may be a moiety comprising a conjugation group suitable for “click chemistry” that generates covalent bonds quickly and reliably by reacting with another moiety comprising a “click chemistry” partner group (i.e. a payload comprising a conjugation partner group), for instance, via strain-promoted cycloaddition, [2+3] dipolar cycloaddition, or Diels-Alder cycloaddition.

In one embodiment, the moiety is a moiety comprising a conjugation group that can react to form covalent bonds in the absence of a metal catalyst (“metal-free”) as described e.g. by Becer et al. in “Click Chemistry beyond Metal-Catalysed Cycloaddition” Angewandte Chemie Int. Ed. 2009, 48(27), 4900-4908. Examples of conjugation groups which can react in the absence of a metal catalyst include electron-deficient alkynes, strained alkynes such as cyclooctynes, tetrazines, and azides.

In a preferred embodiment, the moiety is (ii) a moiety comprising a conjugation group selected from an azide (N₃), TZ, TCO, BCN and DBCO, more preferably BCN or DBCO, most preferably DBCO.

According to one embodiment, the payload is (iii) a moiety derived from a drug. Hereinafter are exemplary drugs that can be used as payloads in the compound of the present invention:
(A) Antineoplastic agents such as
   (A1) DNA-alkylating agents, e.g. duocarmycin (including synthetic analogues thereof: adozelesin, carzelesin, bizelesin, KW-2189 and CBI-TMI), nitrogen mustard analogues (e.g. cyclophosphamide chlorambucil, melphalan, chlormethine, ifosfamide, trofosfamide, prednimustine, bendamustine, chlornaphazine, estramustine, mechlorethamine, mechlorethamine oxide hydrochloride, mannomustine, mitolactol, novembichin, phenesterine, uracil mustard), alkyl sulphonates (e.g. busulfan, treosulfan, mannosulfan, improsulfan and piposulfan), ethylene imines (e.g. thiotepa, triaziquone, carboquone), nitrosoureas (e.g. carmustine, lomustine, semustine, streptozocin, chlorozotocin, fotemustine, nimustine, ranimustine), epoxides (e.g. etoglucid), other alkylating agents (e.g. mitobronitol, pipobroman, temozolomide, dacarbazine);
   (A2) Topoisomerase inhibitors, e.g. doxorubicin, morpholino-doxorubicin, cyanomorpholino-doxorubicin, 2-pyrrolino-doxorubicin, deoxydoxorubicin, etoposide, etoposide phosphate, irinotecan and metabolites thereof such as SN-38, teniposide, topotecan, resveratrol, epipodophyllins (e.g. 9-aminocamptothecin, camptothecin, crisnatol, daunomycin, mitoxantrone, novantrone, retinoic acids (retinols), 9-nitrocamptothecin (RFS 2000));
   (A3) RNA-polymerase II inhibitors, e.g. alpha-amanitin, other amatoxins;
   (A4) DNA-cleaving agents, e.g. calicheamicin;
   (A5) Antimitotic agents or microtubule disruptors, e.g. vinca alkaloids (e.g. vincristine, vinblastine, vindesine, vinorelbine, navelbin, vinflunide, vintafolide), taxanes (e.g. paclitaxel, docetaxel, paclitaxel polyglumex, cabazitaxel) and their analogs, maytansinoids (e.g. DM1, DM2, DM3, DM4, maytansine and ansamitocins) and their analogs, cryptophycins (e.g. cryptophycin 1 and cryptophycin 8), epothilones, eleutherobin, discodermolide, bryostatins, dolostatins, auristatins (e.g. monomethyl auristatin E (MMAE), monomethyl auristatin F), tubulysins, cephalostatins; pancratistatin, sarcodictyin, spongistatin, demecolcine, mitomycins;
   (A6) Anti-metabolites, e.g. DHFR inhibitors (e.g. methotrexate, trimetrexate, denopterin, pteropterin, aminopterin (4-aminopteroic acid) or other folic acid analogues such as raltitrexed, pemetrexed, pralatrexate), IMP dehydrogenase inhibitors (e.g. mycophenolic acid, tiazofurin, ribavirin, EICAR), ribonucleotide reductase inhibitors (e.g. hydroxyurea, deferoxamine), pyrimidine analogs (e.g. cytarabine, fluorouracil, 5-fluorouracil and metabolites thereof, tegafur, carmofur, gemcitabine, capecitabine, azacitidine, decitabine, fluorouracil combinations, tegafur combinations, trifluridine combinations, cytosine arabinoside, ancitabine, floxuridine, doxifluridine), uracil analogs (e.g. 6-azauridine, deoxyuridine), cytosine analogs (e.g. enocitabine), purine analogs (e.g. azathioprine, fludarabine, mercaptopurine, thiamiprine, thioguanine, cladribine, clofarabine, nelarabine), folic acid replenisher such as folinic acid;
   (A7) A kinesin spindle protein inhibitor, e.g. filanesib;
   (A8) Kinase inhibitors, e.g. ipatasertib, BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, afatinib, vemurafenib, crizotinib, regorafenib, masitinib, dabrafenib, trametinib, ibrutinib, ceritinib, lenvatinib, nintedanib, cediranib, palbocidib, osimertinib, alectinib, alectinib, rociletinib, cobimetinib, midostaurin, olmutinib, E7080 (anti-VEGFR2), mubritinib, ponatinib (AP24534), bafetinib (INNO-406), bosutinib (SKI-606), cabozantinib, vismodegib, iniparib, ruxolitinib, CYT387, tivozanib, ispinesib, temsirolimus, everolimus, ridaforolimus;
   (A9) A nicotinamide phosphoribosyltransferase inhibitor, e.g. CAS No. 2241014-82-2;
   (A10) A matrix metallopeptidase 9 inhibitor, e.g. derivatives of CGS27023A;
   (A11) A phosphatase inhibitor, e.g. mycrocystin-LR;
(B) Immunomodulatory agents including immunostimulants, immunosuppressants, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, prednisone, triamcinolone acetonide, beclometasone dipropionate), DHEA, hydroxychloroquine, meloxicam, methotrexate, mofetil, mycophenylate, sirolimus, tacrolimus, everolimus, fingolimod, ibrutinib, imiquimod, resiquimod, cytokines, peptidic immunomodulators such as TLR agonists (e.g. CpG oligonucleotides);
(C) Anti-infectious disease agents including antibacterial drugs, antimycobacterial drugs and antiviral drugs. A non-limiting example of antibiotic used in an antibiotic-antibody drug conjugate is rifalogue, i.e. a rafamycin derivative;
(D) Radioisotopes, metabolites, pharmaceutically acceptable salts, and/or prodrugs of any of the aforementioned agents (A) to (C).

According to one embodiment, the payload is a moiety derived from exatecan, PNU-159682, DM4, amanitin, duocarmycin, auristatin, maytansine, tubulysin, calicheamicin, SN-38, taxol, daunomycin, vinblastine, doxorubicine, methotrexate, pyrrolobenzodiazepine, pyrrole-based kinesin spindle protein (KSP) inhibitors, indolino-benzodiazepine dimers, or radioisotopes and/or pharmaceutically acceptable salts thereof. If the compound of formula (1) contains more than one payload, each P¹ can be independently selected from the aforementioned moieties. Preferably, the payloads are identical to each other.

In one embodiment, the payload is (iv) a (solubilizing) moiety comprising one or more solubilizing groups, e.g., 1 to 10 solubilizing groups, preferably 1, 2, 3, 4, or 5 solubilizing groups.

The covalent attachment of one or more solubilizing moieties can significantly enhance the water solubility of the compound, e.g., antibody or antibody fragment, to which it is attached. The solubilizing effect exerted by the one or more solubilizing moieties may enable to achieve higher DAR-values while keeping favourable pharmacokinetic (PK) properties. In some aspects, the solubilizing moiety can mask the hydrophobicity of a payload, such as vedotin (MMAE) or DM4, whereby the physical association characteristics and/or PK properties of the resulting modified antibody or antibody fragment are improved. In some further aspects, linkage of the one or more solubilizing moieties to an antibody can result in changes including, for example, increased (prolonged) serum half-life, and modulated physical association characteristics, such as reduced aggregation and multimer formation.

The solubilizing group(s) can be any group(s) capable of enhancing water solubility. Preferably, each solubilizing group is independently selected from the group consisting of polyalkylene oxide groups, such as PEO or PPO groups, and moieties comprising one or more ionic groups, such as amino acids, e.g., Lys, Glu, Asp, His, Arg, Dap, Dab, Orn, Aad. Preferably, the solubilizing moiety comprises one or more C₂₋₃ polyalkylene oxide groups, e.g., PEO groups, comprising from 4 to 600, preferably from 10 to 200, more preferably from 15 to 80 repeating units.

In a preferred embodiment, the solubilizing moiety comprises one or more PEO groups, wherein preferably each PEO group independently comprises from 4 to 600, more preferably from 10 to 200, even more preferably from 15 to 80 repeating units, such as 30 to 50, e.g., 40 repeating units.

There is no particular limitation as to the general arrangement of the solubilizing groups in the solubilizing moiety. Hence, the solubilizing moiety can have a linear structure, e.g., in which several solubilizing groups are arranged in a random or block-wise manner, or a branched structure, e.g., in which several solubilizing groups are attached to a core molecule, such as pentaerythritol or glycerol, in a graft or dendrimeric manner. The solubilizing moiety can also comprise several blocks, each block independently having a linear or branched structure.

In one aspect, the solubilizing moiety comprises one or more solubilizing groups arranged in a linear, block-wise manner. For example, the solubilizing moiety can comprise a structure represented by -(So¹)-(So²)-[ . . . ](Soⁿ), wherein each So¹ to Soⁿ represents a solubilizing group, such as a polyalkylene oxide group, e.g., a PEO group having from 4 to 600 repeating units, or a moiety comprising one or more ionic groups, such as Arg, and n is an integer of 1 to 20, e.g., 1 to 10, with the proviso that directly connected polyalkylene oxide groups of the same structure are to be regarded as multiple repeating units of the same solubilizing group (and not as adjacent So groups). That is, adjacent polyalkylene oxide groups must be of different structure and/or be connected via a functional group like —C(G)-G- or the like in order to be treated as separate So groups.

In one embodiment, P¹ is a moiety represented by the following formula (12a):

—X⁶—(So-(X⁶)ₙ₂₀-)ₙ₁₈-X⁷ (12a)

In formula (12a), n18 is an integer of 1 to 20, preferably 1 to 10, more preferably 1 to 6, such 2 or 4 and n20 is 0 or 1.

Each So represents a solubilizing group, wherein preferably each So is independently selected from a polyalkylene oxide group, e.g., a PEO group having from 4 to 600 repeating units, and a moiety comprising one or more ionic groups, e.g., Arg.

Each X⁶ is independently selected from a single covalent bond, —(C═O)—, and —N(R)— in which R represents a hydrogen atom, an alkyl group or a cycloalkyl group.

X⁷ represents an alkyl group having 1 to 6 carbon atoms such as a methyl group, a carbonyl-containing group such an acetyl group or a group of formula —(CH₂)ₙ₄—CO₂H, a thiocarbonyl-containing group, a group of formula —(CH₂)ₙ₄₀R, a group of formula —(CH₂)ₙ₄—SO₃H, or an amino-containing group such as a group of formula —(CH₂)ₙ₄—(C═X)—N(R′)(R) or —(CH₂)ₙ₄—N(R′)(R), in which X is 0 or S, R and R′ are each independently selected from a hydrogen atom, an alkyl group or a cycloalkyl group, and n4 is an integer of 1 to 6.

Preferably, X⁷ is a methyl group, or a group represented by the following formula (12a′):

—(CH₂)ₙ₅—(C═X)N(R)—(CH₂)ₙ₆—(C═X)N(H)(R) (12a′)
wherein,
each X is independently selected from O and S, preferably O,
each R is independently selected from a hydrogen atom, an alkyl group and a cycloalkyl group, and
n5 and n6 are each independently an integer of 1 to 6, preferably 1 or 2.

Most preferably, X⁷ is a methyl group.

In one embodiment, P¹ is a moiety represented by the following formula (12b):

—X⁶—((CH₂CH₂O)ₙ₁₉-(AA⁴)ₙ₂₀)ₙ₂₁—X⁷ (12b)

In formula (12b), X⁶ and X⁷ are as defined in formula (12a).

AA⁴ represents a moiety comprising one or more ionic groups, preferably an amino acid selected from Asp, Glu, Lys, Arg, His and, more preferably Arg.

n19 is an integer of 0 to 600, preferably 10 to 200, more preferably 15 to 80, such as 30 to 50, e.g., 40; n20 is 0 or 1; and n21 is 1 to 10, preferably 1 to 5, more preferably 1 to 3.

In a preferred embodiment, P¹ is a moiety represented by the following formula (12c):

—X⁶—(CH₂CH₂O)ₙ₁₉′—X⁷ (12c)

In formula (12c), X⁶ and X⁷ are as defined in formula (12a), and n19′ is an integer of 4 to 600, preferably 10 to 200, more preferably 15 to 80, such as 30 to 50, e.g., 40.

In another aspect, the solubilizing moiety comprises one or more solubilizing groups attached to a core molecule, such as pentaerythritol or glycerol, in an untethered, graft or dendrimeric manner. For example, the solubilizing moiety can have a graft structure represented by:

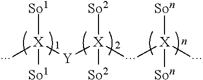

wherein X is a multivalent, e.g., tri- or tetravalent, group, Y is a divalent group, each So¹ to Soⁿ is independently selected to be a solubilizing group, such as a polyalkylene oxide group, e.g., a PEO group having from 4 to 600 repeating units, or a moiety comprising one or more ionic groups, and n is an integer of 1 to 20, e.g., 1 to 10;
or a tree-like, dendrimeric structure represented by:

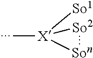

wherein X′ is a multivalent (branching) group, each So¹ to Soⁿ is independently selected to be a solubilizing group, such as a polyalkylene oxide group, e.g., a PEO group having from 4 to 600 repeating units, or a moiety comprising one or more ionic groups, and n is an integer of 1 to 20, e.g., 1 to 10.

In one embodiment, P¹ is a moiety represented by the following formula (12d):

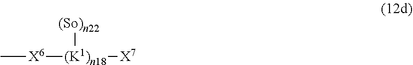

In formula (12d), X⁶, X⁷, and n18 are as defined in formula (12a); and n22 is 1 or 2, preferably 1.

Each So is independently selected to be a solubilizing group, wherein preferably each So is independently selected from a polyalkylene oxide group, e.g., a PEO group having from 4 to 600 repeating units, and a moiety comprising one or more ionic groups. Most preferably, each So represents a PEO group having from 4 to 600 repeating units

Each K¹ is independently selected to be a tri- or tetravalent group, e.g., a group derived from a core molecule containing one or two functional groups each allowing covalent attachment to a solubilizing group and two further functional groups allowing covalent attachment to adjacent moieties.

In another embodiment, P¹ is a moiety represented by the following formula (12e):

—X⁶—K²(-So)ₙ₁₈ (12e)

In formula (12e), X⁶ and n18 are as defined in formula (12a).

Each So is independently selected to be a solubilizing group, wherein preferably each So is independently selected from a polyalkylene oxide group, e.g., a PEO group having from 4 to 600 repeating units, and a moiety comprising one or more ionic groups. Most preferably, each So represents a PEO group having from 4 to 600 repeating units

K² represents a (n18+1)-valent group, such as a trivalent or tetravalent group, which is covalently attached to X⁶, as well as to two or more solubilizing groups thereby forming a branched (tree-like or dendrimeric) structure. Preferably, K² is represented by the following formula (12e′):

*—CH(R₁₀—**)ₙ₁₈ (12e′)
wherein,
n18 is as defined above;
R₁₀ is a group of formula —(CH₂)ₘ₅—** or —(CH₂)ₘ₅R₁₁—**, wherein R₁₁ is selected from the group consisting of —NH—**, —(C═X)R₁₂—** and —NH(C═X)R₁₂—**, and is preferably —NH—**;
R₁₂ is —(CH₂)ₘ₆—**, —(CH₂)ₘ₆S—**, —CH(R₁₃—**)₂, or —(CH₂)ₘ₆NH—**;
R₁₃ is —CH₂S—**, (CH₂)ₘ₇—**, or —(CH₂)ₘ₇R₁₄—**;
R₁₄ is —NH(C═X)ₘ₁₅S—**;
each X is independently selected from O and S, and is preferably O;
* indicates covalent attachment to X⁶;
** indicates covalent attachment to a solubilizing group So;
m5 is 1, 2 or 3; m6 and m7 are each independently selected from 0, 1, 2 and 3;
each m5, m6 and m7 being preferably 1.

In one embodiment, P¹ is a moiety represented by one of the following formulae: —(CH₂CH₂O)ₙ₁₉—CH₂CH₃, —(CH₂CH₂O)ₙ₁₉—(C═O)CH₃, —(CH₂CH₂O)ₙ₁₉—(C═O)NH₂, —(CH₂CH₂O)ₙ₁₉—CH₂—N(H)(R), —(CH₂CH₂O)ₙ₁₉—(CH₂)₂—N(H)(R), —(CH₂CH₂O)ₙ₁₉—(CH₂)₂—(C═O)N(R)—CH₂—(C═O)N(H)(R), —(CH₂CH₂O)ₙ₁₉—(CH₂)₂—(C═O)N(R)—(CH₂)₂—(C═O)N(H)(R), —(C═O)—(CH₂CH₂O)ₙ₁₉—CH₂CH₃, —(C═O)—(CH₂CH₂O)ₙ₁₉—(C═O)CH₃, —(C═O)—(CH₂CH₂O)ₙ₁₉—(C═O)NH₂, —(C═O)—(CH₂CH₂O)ₙ₁₉—CH₂—N(H)(R), —(C═O)—(CH₂CH₂O)ₙ₁₉g-(CH₂)₂—N(H)(R), —(C═O)—(CH₂CH₂O)ₙ₁₉—(CH₂)₂—(C═O)N(R)—CH₂—(C═O)N(H)(R), —(C═O)—(CH₂CH₂O)ₙ₁₉—(CH₂)₂—(C═O)N(R)—(CH₂)₂—(C═O)N(H)(R), —N(H)(R)-(CH₂CH₂O)ₙ₁₉—CH₂CH₃, —N(H)(R)-(CH₂CH₂O)ₙ₁₉—(C═O)CH₃, —N(H)(R)-(CH₂CH₂O)ₙ₁₉—(C═O)NH₂, —N(H)(R)-(CH₂CH₂O)ₙ₁₉—CH₂—N(H)(R), —N(H)(R)-(CH₂CH₂O)ₙ₁₉—(CH₂)₂—N(H)(R), —N(H)(R)-(CH₂CH₂O)ₙ₁₉—(CH₂)₂—(C═O)N(R)—CH₂—(C═O)N(H)(R), and —N(H)(R)-(CH₂CH₂O)ₙ₁₉—(CH₂)₂—(C═O)N(R)—(CH₂)₂—(C═O)N(H)(R), wherein n19 and R are as defined above. Preferably, R is a hydrogen atom and n19 is an integer of 15 to 80, such as 30 to 50, or 35 to 45, in particular 40.

### 3.3 Reactive Moiety (Y)

The compound of the present invention comprises a reactive moiety (Y) which can react, e.g. via a nucleophilic substitution reaction, with the side chain of an amino acid exposed at the surface of an antibody or antibody fragment. Preferably, the reactive moiety is capable of reacting with the side chain of a lysine residue. This reaction leads to the covalent attachment of the group (P)—which comprises one or more payloads (P¹)—to said antibody or antibody fragment, with the concomitant release of the peptide (V). When the reactive moiety (Y) reacts with the side chain of an amino acid exposed at the surface of an antibody or antibody fragment to form a covalent bond, a covalent bond within Y or between Y and V is spontaneously cleaved to release the peptide (without need for further chemical reaction such as hydrolysis or reduction).

The reactive moiety comprises a reactive center (RC) that is capable of reacting with the side chain of an amino acid, preferably with the side chain of a lysine residue, for instance via a nucleophilic substitution reaction. Preferably, the reactive center is electrophilic. Non-limiting examples of electrophilic reactive centers capable of reacting with the side chain of an amino acid include C═O and C═S. A preferred reactive center is carbonyl (C═O) or thiocarbonyl (C═S), and particularly preferred is carbonyl (C═O).

Covalently attached to one side of the reactive center is a moiety (F1) through which the reactive center is attached to the group (P). Covalently attached to the other side of said reactive center is a moiety (F2) through which the reactive center is attached to the peptide (V), optionally via a spacer (S). Accordingly, the reactive moiety may be represented by the following formula (4a):

*′-F1-RC-F2-*** (4a)
wherein,
RC is a reactive centre, preferably an electrophilic reactive centre, and more preferably a group selected from C═O and C═S;
F1 is a single covalent bond, an atom, or a group of atoms; preferably a CH₂ or NH group of atoms or an atom selected from O and S, or a group of atoms comprising one or more atoms selected from C, N, O, and S; more preferably a CH₂ group of atoms or an atom selected from O and S;
F2 represents an atom, or a group of atoms; preferably an atom selected from
O, and S, or a group of atoms comprising one or more atoms selected from C, N, O, and S; more preferably an atom selected from O and S;
*′ indicates covalent attachment to the group (P), and
*** indicates covalent attachment to the peptide (V).

F1 and F2 may be identical atoms or groups of atoms. However, preferably the atom or group of atoms that constitute F2 make it a better/preferred leaving group than/to F1 in a nucleophilic substitution reaction. This ensures that when the reactive center (RC) reacts with the side chain of an amino acid residue, for instance with the side chain of a lysine residue, on the antibody or antibody fragment via a nucleophilic substitution reaction, F2 is the preferred leaving group; resulting in the payload being attached to the antibody or antibody fragment and not the vector/spacer construct.

To ensure that F2 is a better or more preferred leaving group than F1 in a nucleophilic substitution reaction, especially if F1 and F2 are the same atom or group of atoms, F2 may be linked to a modifying group (M) wherein, M is a group capable of modulating the electronegativity and/or stability of the neighbouring moiety F2 e.g. by withdrawing or donating electrons (and thereby also the reactivity of the RC). Accordingly, in an embodiment, the reactive moiety may be represented by the following formula (4b):

*′-(F1-RC-F2)-M-*** (4b)
wherein,
RC, F1, F2, *′, and *** are as defined in formula (4a) above; and
M is a group capable of modulating the electron density and stability of F2, preferably a group capable of withdrawing electrons.

In one preferred embodiment, the reactive moiety is represented by formula (4b).

If a modifying group (M) is present in the reactive conjugate of the invention, the peptide released during the one step reaction of Y (specifically the RC) with the side chain of an amino acid exposed at the surface of the antibody or antibody fragment comprises the moiety H-F2-M-***.

In an embodiment, M is represented by the following formula (4c):

***‘--M’-B-E--*** (4c)
wherein,
M′ is a moiety derived from succinimide or an aryl group having 6, 10 or 14 ring members and 1, 2 or 3 condensed rings, respectively, or a heteroaryl group having 5 to 14 ring members, 1, 2 or 3 condensed rings and 1 to 4 heteroatoms independently selected from N, O and S, each of the aforementioned groups being optionally substituted with one or more substituents. Preferably, M′ is a divalent group derived from a phenyl group, a naphthyl group, a pyridyl group, a quinolinyl group, an isoquinolinyl group and a benzotriazolyl group, each of the aforementioned groups being optionally substituted with one or more substituents and each substituent being preferably selected from —F, —Br, —Cl, —I, —NO₂, —CN, —C₁₋₆-alkyl, —C₁₋₆-alkoxy such as —O—CH₃ or —O—CH₂CH₃, —C₁₋₆-amido such as —CH₂—C(O)NH₂, and combinations thereof such as —CCl₃, —CF₃ or —CH₂NO₂;
B is selected from:
   a single covalent bond, O, S, or a group of atoms NR′, wherein R′ represents a hydrogen atom, —OH, an alkyl group, a cycloalkyl group, a C₂₋₆-alkenylene group, or a C₂₋₆-alkynylene group;
   a group represented by the following formula (4d):

—X¹—(CH₂CH₂O)ₙ₂—CH₂CH₂—^{♦♦} (4d)
wherein,
X¹ is —C(O)NH—, —CO—, —NH— or —S—; preferably —C(O)NH—;
n2 is an integer of 1 to 24, preferably 1 to 10, more preferably 1 to 3;
♦ indicates covalent attachment to M′, and
♦♦ indicates covalent attachment to E;
a peptidic group having 6 to 25 amino acids in the main chain, e.g. 9 amino acids in the main chain, each amino acid being preferably selected from Pro, Gly, Ala, Asn, Asp, Thr, Glu, Gln and Ser; more preferably from Pro, Gly and Ser;
and any combinations thereof;
wherein, B is preferably a single covalent bond or a group having the general formula (4d), more preferably a single covalent bond;
E is C═O, C═S or C(═NR″), wherein R″ represents a hydrogen atom, OH, an alkyl group, a cycloalkyl group, S═O, or S(═O)₂; preferably E is C═O;
***′ indicates covalent attachment to F2; and
*** indicates covalent attachment to the peptide (V).

If a spacer (S) is present between F2 and the peptide (V), the spacer is a divalent group, preferably comprising one or more atoms selected from carbon, nitrogen, oxygen, phosphorus and sulfur.

In an embodiment, the spacer comprises a polyalkylene oxide group having from 1 to 24 repeating units and 2 or 3 carbon atoms per repeating unit, preferably 1 to 10 repeating units; and preferably a group represented by the formula —NH—(CH₂CH₂O)ₙ₁—CH₂CH₂-E′- wherein n1 is an integer of 0 to 24, e.g. 1 to 10, and E′ is (C═O) or (C═S).

According to one embodiment, the moiety (F1-RC-F2) is represented by one of the formulae (4a′) to (4m′) and/or M is independently represented by one of the following formulae (5a) to (5h′):

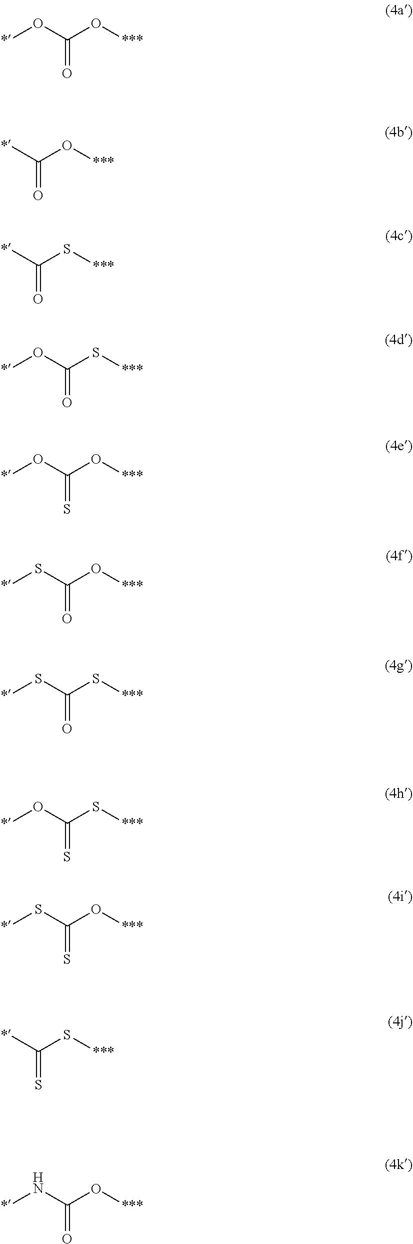

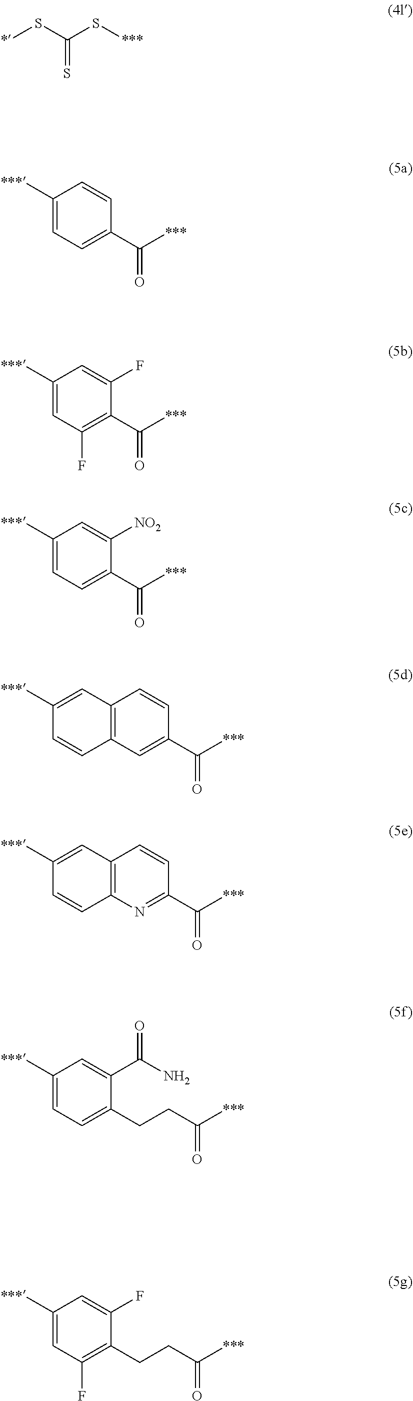

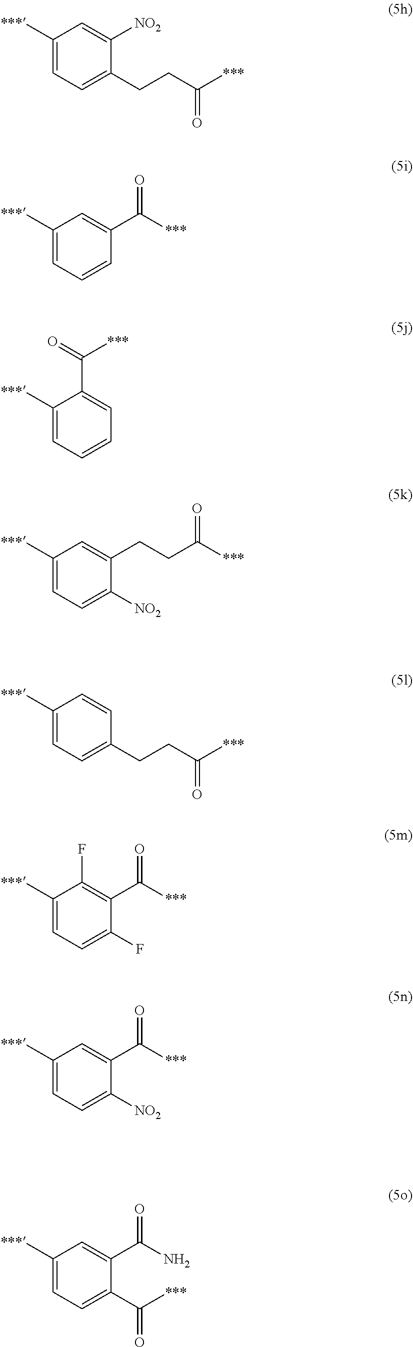

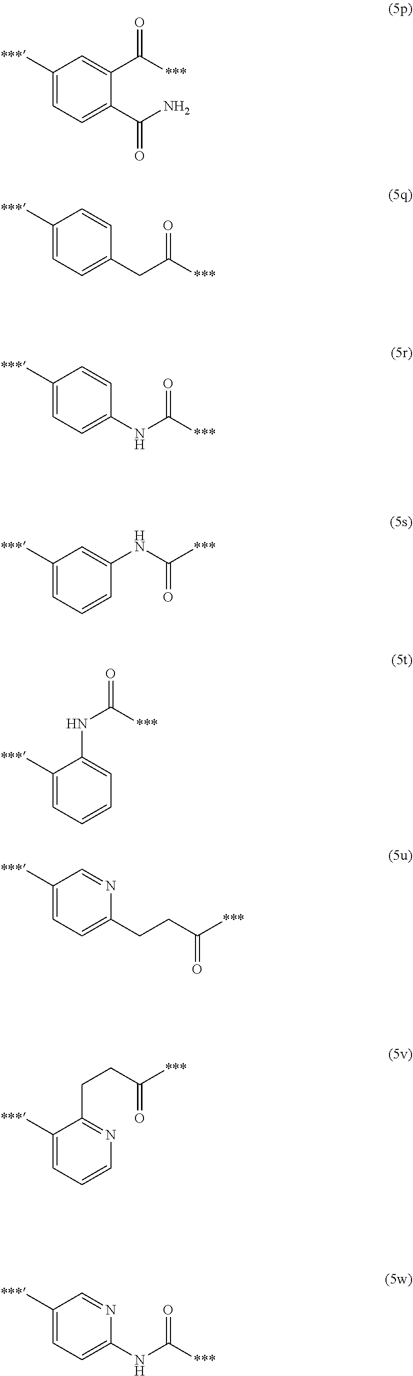

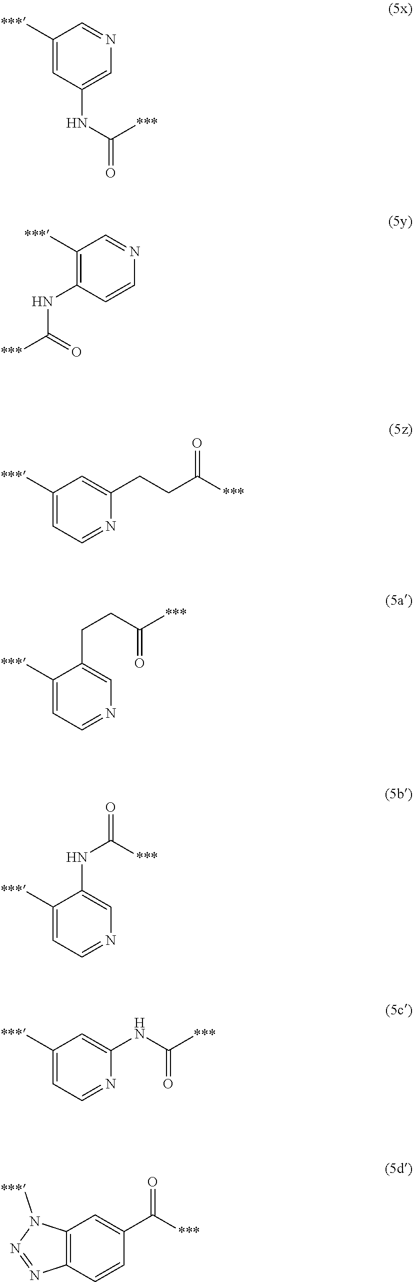

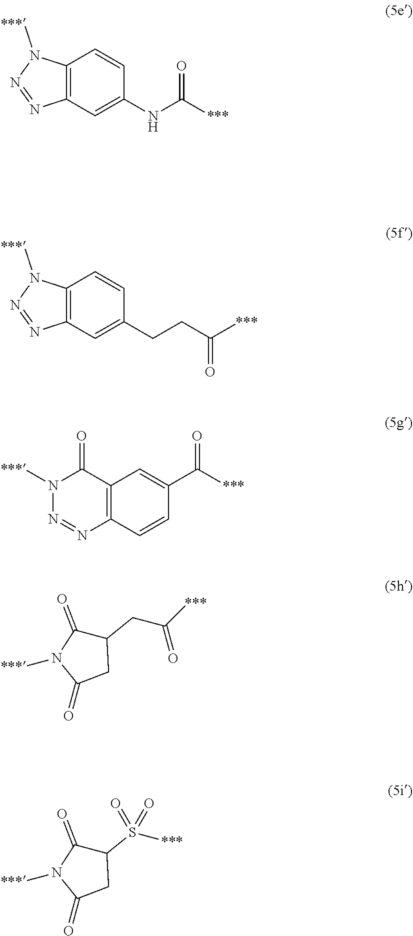

wherein,
*′ indicates covalent attachment to the group (P),
*** indicates covalent attachment to the peptide (V), or to the group (M) if M is present, and
***′ indicates covalent attachment to F2 of the reactive moiety (Y).

In preferred embodiments, the reactive moiety is represented by one of the following formulae (6a) to (6l′):

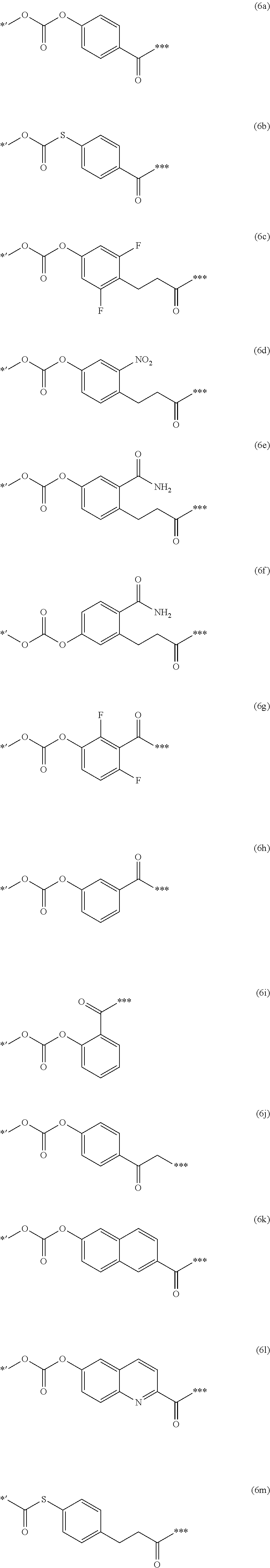

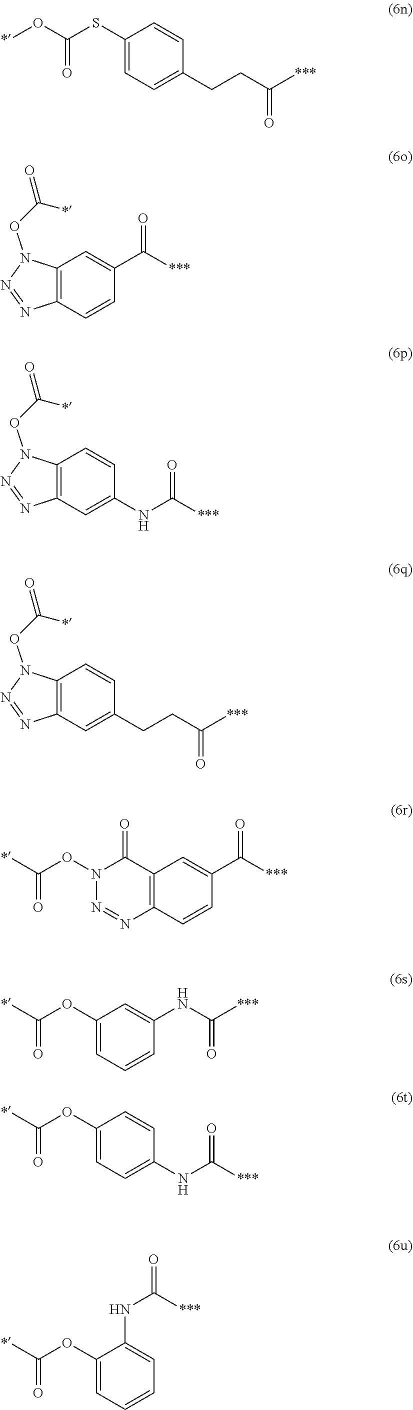

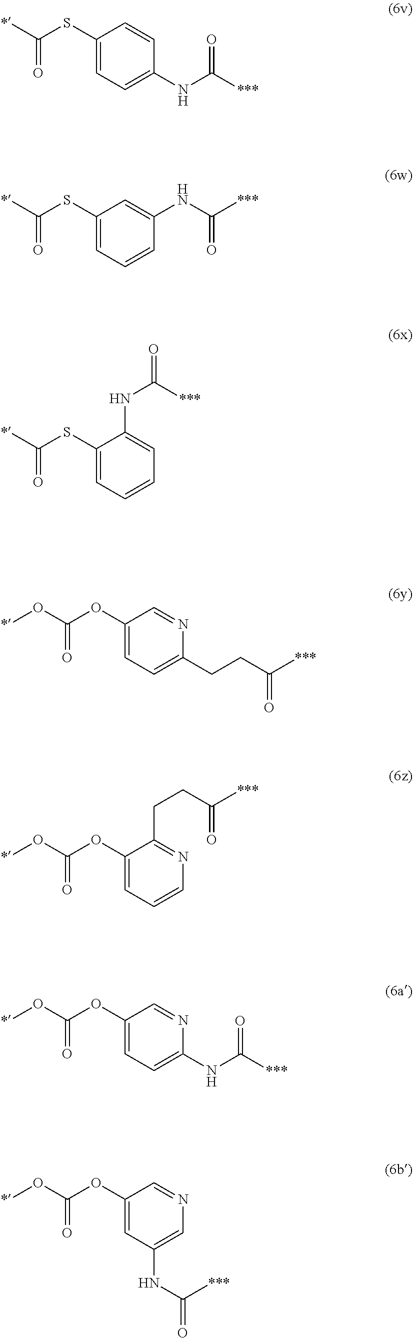

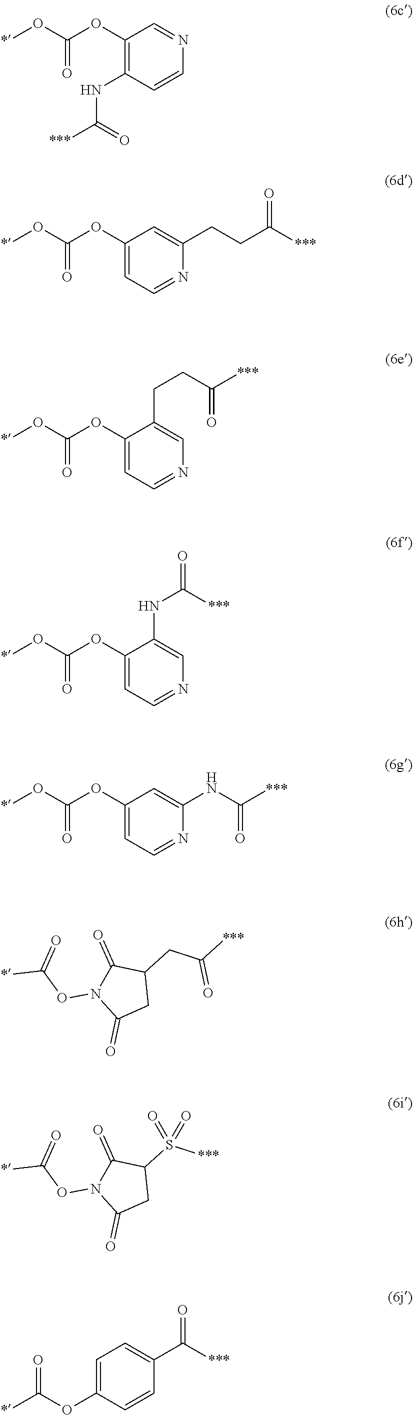

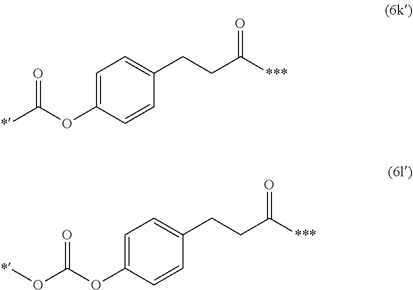

wherein,
*′ indicates covalent attachment to the group (P), and
*** indicates covalent attachment to the peptide (V).

More preferably, the reactive moiety is represented by one of the formulae (6a), (6l′), (6m) and (6j′). Even more preferably, the reactive moiety is represented by formula (6a) or (6l′). Most preferably, the reactive moiety is represented by formula (6a).

### 3.4 Peptide (V)

The compound of the present invention comprises a peptide (V) comprising a vector (or “ligand”) capable of interacting with (binding to) the fragment crystallizable (Fc) region of an antibody or fragment thereof, the antibody fragment being optionally incorporated into an Fc-fusion protein. The interaction of the vector with the Fc region leads to an increase in the concentration of the reactive moiety in proximity to the side chain of an amino acid exposed at the surface of the antibody or antibody fragment, leading to covalent attachment of the payload to the side chain. In some aspects, the interaction of the vector with the Fc region leads to a targeting effect insofar that the reactive moiety will react with the side chain of a specific amino acid exposed at the surface of the antibody or antibody fragment (e.g. the lysine residue at position 248), leading to the regioselective attachment of the group (P)—which may comprise one or more payloads—to the antibody or antibody fragment. In one embodiment, the peptide is a vector capable of interacting with the Fc region of an antibody or fragment thereof, the antibody fragment being optionally incorporated into an Fc-fusion protein.

Vectors capable of interacting with the Fc region of antibodies or fragments thereof are known in the art and are described e.g. in Choe et al. Materials 2016, 9, 994. Suitable vectors are also disclosed in WO 2018/199337 A1. Non-limiting examples of vectors capable of interacting with the Fc region of antibodies or fragments thereof include protein Z and Fc-III. In particular, the cyclic peptide Fc-III has been described as a peptidic vector/ligand having high affinity for the Fc region of IgG proteins with a reported dissociation constant K_{d} of about 16 nm (DeLano et al. Science 2000, 287, 1279-1283).

In one embodiment, the peptide comprises a sequence of 11 to 17 amino acids, e.g. 13 to 17 amino acids, which is preferably cyclic.

In one preferred embodiment, the peptide is represented by the following formula (7a):

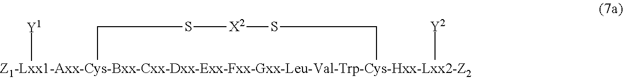

wherein,
   Bxx represents an amino acid selected from an amino acid with an amino group-containing side chain, Ala, Tyr, homo-tyrosine (hTyr), and meta-tyrosine (mTyr); preferably an amino acid selected from Lys, homo-lysine (hLys), ornithine (Orn), 2,3-diaminopropionic acid (Dap), 2,4-diamiobutyric acid (Dab), Ala, Tyr, hTyr, and mTyr; more preferably Ala;
   Cxx, Dxx, Exx, Fxx, Gxx each independently represent an amino acid;
   Axx represents an amino acid, a dicarboxylic acid, or a peptide moiety represented by the following formula (8a):

---Axx1-Axx2-Axx3--- (8a)
wherein, in formula (8a),
Axx1 represents a single covalent bond, or an amino acid such as Arg;
Axx2 represents an amino acid such as Gly or Cys, preferably Gly; and
Axx3 represents an amino acid such Asp or Asn;
Hxx represents an amino acid, or a peptide moiety represented by the following formula (8b):

---Hxx1-Hxx2-Hxx3--- (8b)
wherein, in formula (8b),
Hxx1 represents an amino acid such as Thr;
Hxx2 represents a single covalent bond or an amino acid such as Tyr or Cys; and
Hxx3 represents a single covalent bond, or an amino acid such as His; and the side chain of Axx2 may be covalently attached to the side chain of Hxx2 to form a ring;
wherein preferably both Hxx2 and Hxx3 represent single covalent bonds;
if Axx2 is Cys and Hxx2 is Cys, preferably the side chains of Axx2 and Hxx2 are linked together to form a group of formula —(S—X³—S)—, wherein X³ represents a single covalent bond or a divalent group comprising one or more atoms selected from carbon, nitrogen and oxygen such as a divalent maleimide group, a divalent acetone group or a divalent arylene group; preferably X³ represents a single covalent bond;
Lxx1 and Lxx2 each independently represent a single covalent bond or a trifunctional amino acid such as a diamino-carboxylic acid; with the proviso that at least one of Lxx1 and Lxx2 is a single covalent bond;
Z₁ represents a group selected from:
   a group covalently attached to the N-terminus of Axx, if Lxx1 is a single covalent bond, which is selected from a hydrogen atom, a carbonyl-containing group such as an acetyl group, a C₂₋₃ polyalkylene oxide-containing group, e.g., a PEG-containing group, and a group derived from a compound containing a conjugation group such as biotin, DBCO, TCO, TZ, BCN, an alkyne, an azide, a bromoacetamide, a maleimide or a thiol, wherein said conjugation group is optionally attached via a spacer (S¹);
   a group covalently attached to the N-terminus of Lxx1, if Lxx1 is a trifunctional amino acid and Y¹ is attached to the side chain of Lxx1, which is selected from a hydrogen atom, a carbonyl-containing group such as an acetyl group, and a C₂₋₃ polyalkylene oxide-containing group, e.g., a PEG-containing group; and
   a hydrogen atom or a C₂₋₃ polyalkylene oxide-containing group, e.g., a PEG-containing group, covalently attached to the side chain of Lxx1, if Lxx1 is a trifunctional amino acid and Y¹ is covalently attached to the N-terminus of Lxx1;
Z₂ represents a group selected from:
   a group covalently attached to the C-terminus of Hxx, if Lxx2 is a single covalent bond, which is selected from —N(H)(R) wherein R represents a hydrogen atom, an alkyl group or a cycloalkyl group, a C₂₋₃ polyalkylene oxide-containing group, e.g., a PEG-containing group, and a group derived from a compound containing a conjugation group derived from biotin, DBCO, TCO, TZ, BCN, an alkyne, an azide, a bromoacetamide, a maleimide or a thiol, wherein said conjugation group may optionally be attached via a spacer (S1);
   a group covalently attached to the C-terminus of Lxx2, if Lxx2 is a trifunctional amino acid and Y² is covalently attached to the side chain of Lxx2; preferably a C₂₋₃ polyalkylene oxide-containing group, e.g., a PEG-containing group, a group represented by N(H)(R) wherein R represents a hydrogen atom, an alkyl group, or a cycloalkyl group; and
   a hydrogen atom or a C₂₋₃ polyalkylene oxide-containing group, e.g., a PEG-containing group, covalently attached to the side chain of Lxx2, if Lxx2 is trifunctional amino acid and Y² is covalently attached to the C-terminus of Lxx2;
Y¹ represents a moiety that is only present if Lxx1 is a trifunctional amino acid, wherein Y¹ is covalently attached to the side chain of Lxx1 if Z₁ is attached to the N-terminus of Lxx1, or to the N-terminus of Lxx1 if Z₁ is attached to the side chain of Lxx1;
   wherein Y¹ is derived from a compound containing a conjugation group, which is preferably selected from biotin, DBCO, TCO, TZ, BCN, an alkyne, an azide, a bromoacetamide, a maleimide, and a thiol, and wherein said conjugation group is optionally attached via a spacer (S¹);
Y² represents a moiety that is only present if Lxx2 is a trifunctional amino acid, wherein Y² is covalently attached to the side chain of Lxx2 if Z₂ is attached to the C-terminus of Lxx2, or to the C-terminus of Lxx2 if Z₂ is attached to the side chain of Lxx2;
   wherein Y² is derived from a compound containing a conjugation group, which is preferably selected from biotin, DBCO, TCO, TZ, BCN, an alkyne, an azide, a bromoacetamide, a maleimide, and a thiol, and wherein said conjugation group is optionally attached via a spacer (S¹);
S¹ is a spacer represented by the following formula (8c):

α-X⁴—(CH₂CH₂O)ₙ₂—CH₂CH₂—X⁵—β (8c)
wherein
X⁴ is NH, O or S; preferably NH;
X⁵ is NH or C═O, preferably C═O if X⁵ is covalently attached to the peptide (V);
n2 is an integer of 1 to 46, preferably 1 to 24, most preferably 1 to 12; and
α indicates covalent attachment to Y¹ or Y² if X⁵ is covalently attached to the peptide (V), or covalent attachment to the peptide (V) if X⁵ is covalently attached to Y¹ or Y²; and
β indicates covalent attachment to the peptide (V) if X⁴ is covalently attached to Y¹ or Y², or covalent attachment to Y¹ or Y² if X⁴ is covalently attached to the peptide (V);
X² represents a single covalent bond or a divalent group comprising one or more atoms selected from carbon, nitrogen and oxygen such as a divalent maleimide group, a divalent acetone group or a divalent arylene group; preferably a single covalent bond;
wherein at least one of Axx, Bxx, Cxx, Dxx, Exx, Fxx, Gxx and Hxx, preferably one or more of Bxx, Dxx, Exx, Fxx, and Gxx, more preferably one or more of Bxx, Exx, and Gxx, and most preferably Bxx and/or Exx, represents an amino acid with an amino group-containing side chain, Tyr, hTyr, or mTyr, preferably an amino acid with an amino group-containing side chain, more preferably an amino acid selected from Lys, hLys, Orn, Dap, and Dab, which is covalently attached to the reactive moiety (Y) via its side chain; with the proviso that the peptide (V) does not comprise more than three amino acids with an amino group-containing side chain;
and wherein,
and wherein, if the reactive moiety (Y) is attached to the peptide (V) via a Tyr, hTyr, or mTyr, the reactive moiety (Y) is preferably a moiety of formula (4a) as defined above.

In an embodiment, the moiety Y¹ or Y² is represented by the following formula (8d):

Y³-L¹--**** (8d)
wherein,
Y³ is a moiety derived from a conjugation group selected from biotin, DBCO, TCO, BCN, an alkyne, an azide, a bromoacetamide, a maleimide, and a thiol;
L¹ is a divalent group, preferably comprising one or more atoms selected from C, N, O and S, more preferably comprising a polyethylene oxide group having 1 to 12 repeating units e.g. 4 repeating units; and
**** indicates covalent attachment to Lxx1 or Lxx2.

The linker L¹ is a divalent group, preferably comprising one or more atoms selected from carbon, nitrogen, oxygen, phosphorus and sulfur.

In an embodiment, the linker L¹ can be selected from
(a1) an alkylene group having from 1 to 12 carbon atoms, preferably an alkylene group having from 2 to 6 carbon atoms such as an ethylene group or propylene group;
(b1) a polyalkylene oxide group with 2 or 3 carbon atoms having from 1 to 36 repeating units; preferably a group represented by the formula —NH—(CH₂CH₂O)ₙ₁—CH₂CH₂— wherein n1 is an integer of 0 to 35, e.g. 1 to 20; and
(c1) a peptidic group having 2 to 12 amino acids.

According to one preferred embodiment, at least one of Axx, Cxx, Dxx, Exx, Fxx, Gxx, Hxx, Lxx1 and Lxx2 in formula (7a) is defined as follows:
Axx represents an amino acid selected from Ala, 2,3-diamino-propionic acid (Dap), Asp, Glu, 2-amino suberic acid, α-amino butyric acid, Asn and Gln, a dicarboxylic acid selected from succinic acid, glutaric acid and adipic acid; preferably Ala, Asp or Asn; more preferably Asp; or a peptide moiety of formula (8a), wherein Axx1 is a single covalent bond, Axx2 is Cys, and Axx3 is Asp;
Cxx represents an amino acid selected from Trp, Phe, Tyr, phenyl glycine (Phg), 3-benzothiopen-2-yl-L-alanine, 3-naphthalen-2-yl-L-alanine, 3-biphenyl-4-yl-L-alanine and 3-naphthalen-1-yl-L-alanine; preferably Trp;
Dxx represents an amino acid selected from His, Ala, 3-pyridin-2-yl-L-alanine, meta-tyrosine (mTyr) and Phe; preferably His, Ala or mTyr; more preferably His;
Exx represents an amino acid selected from Ala, 2-amino-butyric acid (Abu), Gly, Leu, Ile, Val, Met, cyclohexyl alanine (Cha), Phe, Thr, Cys, Tyr, and norleucine (Nle); preferably Ala, Nle or Leu; more preferably Leu;
Fxx represents an amino acid selected from Ala, Gly, Asn, Ser, Abu, and Asp; preferably Ala or Gly; more preferably Gly;
Gxx represents an amino acid selected from Ala, Glu, Asp, Gln, His, Arg, Ser, and Asn; preferably Asp or Glu; more preferably Glu;
Hxx represents an amino acid selected from Thr, Ser, Ala, Asn, Val, Abu, Ile, Met, Leu, Pro, and Cys; preferably Thr or Ser; more preferably Thr; or a peptide moiety of formula (8b), wherein Hxx1 is Thr, Hxx2 is Cys, and Hxx3 is a single covalent bond; and
Lxx1 and Lxx2 each independently represent an amino acid selected from Dap, Dab, Lys, Orn and homo-lysine (hLys), preferably an amino acid selected from Dap, Dab, Lys, Orn and hLys.
According to one embodiment, if Dxx or Exx represents an amino acid with an amino group-containing side chain, Tyr, hTyr, or mTyr, which is covalently attached to the reactive moiety (Y) via its side chain, Gxx is preferably Glu, Gln, His, Arg or Asn, and more preferably Gln.

According to one embodiment, the peptide is represented by the following formula (9a):

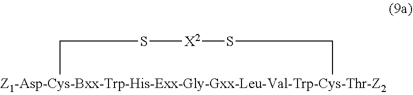

wherein,
   Z₁, Z₂, Bxx, Exx, Gxx and X² are as defined above with respect to formula (7a); and
   at least one of Bxx, Exx and Gxx, preferably Bxx and/or Exx, represents an amino acid with an amino-group containing side chain, Tyr, hTyr, or mTyr; preferably an amino acid with an amino group-containing side chain, and more preferably an amino acid selected from Gln, Lys, hLys, Orn, Dap and Dab, said amino acid being covalently attached to a reactive moiety (Y) via its side chain; and
   if Bxx and/or Exx represents an amino acid with an amino group-containing side chain, Tyr, hTyr, or mTyr, preferably an amino acid with an amino group containing side chain; and more preferably an amino acid selected from Gln, Lys, hLys, Orn, Dap, and Dab, which is covalently attached to the reactive moiety (Y) via its side chain, Gxx is preferably Gln;
and preferably one or two of Bxx, Exx and Gxx is defined as follows:
   Bxx represents an amino acid selected from an amino acid with an amino group-containing side chain, Ala, Tyr, hTyr, and mTyr; preferably Lys, hLys, Orn, Dap, Dab, Ala, Tyr, hTyr, or mTyr; more preferably Ala;
   Exx represents an amino acid selected from Ala, Abu, Gly, Leu, Ile, Val, Met, Cha, Phe, Thr, Cys, Tyr, and Nle; preferably Ala, Nle or Leu; more preferably Leu; and
   Gxx represents an amino acid selected from Ala, Glu, Asp, Gln, His, Arg, Ser, and Asn; preferably Asp or Glu; more preferably Glu.

In one embodiment, the peptide is represented by one of the following formulae (10a) to (10k′):

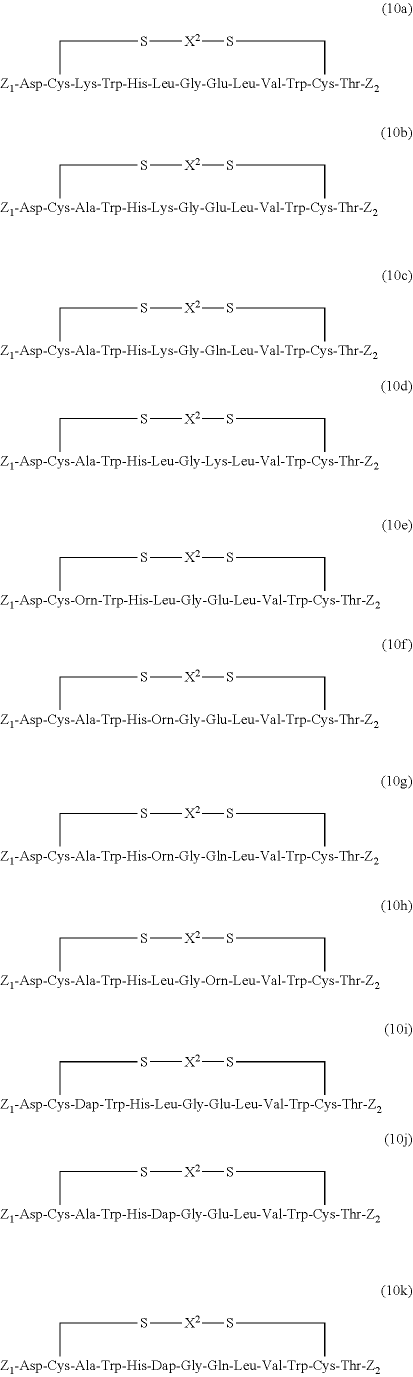

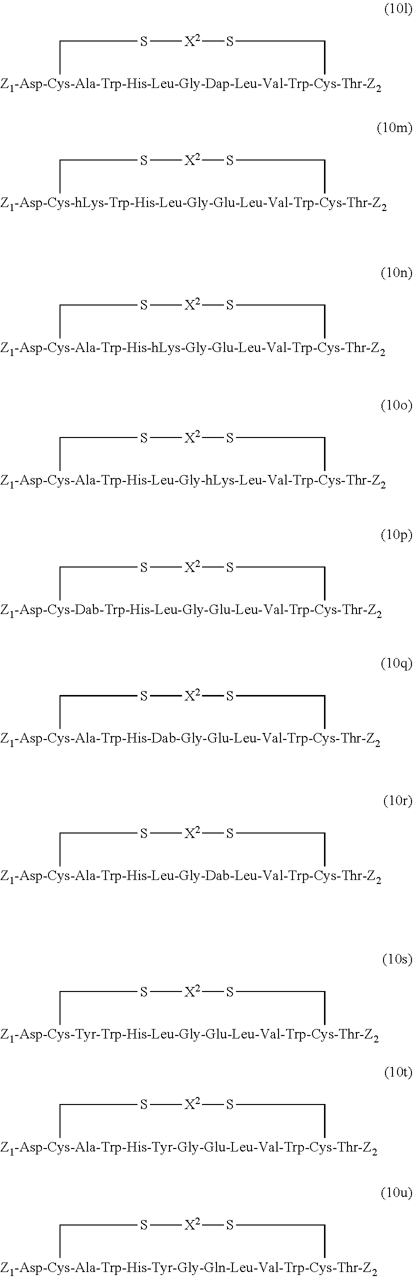

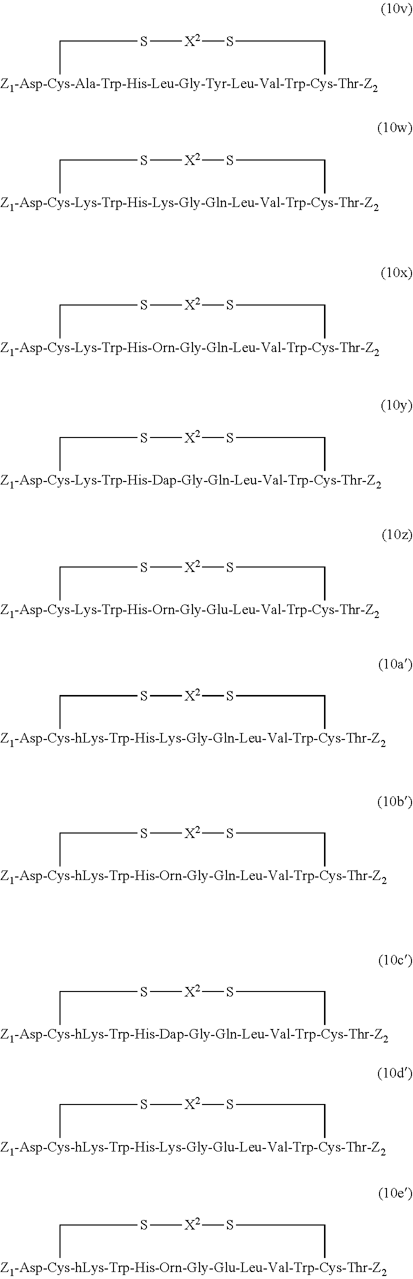

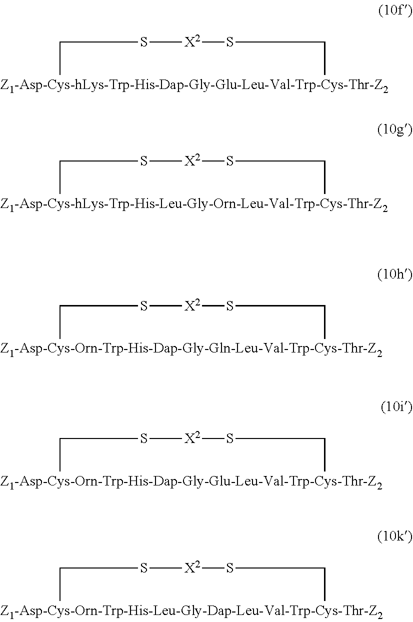

In the formulae (10a) to (10k′) above, Z₁, Z₂, and X² are as described above with respect to formula (7a). In these formulae, the peptide is covalently attached to the reactive moiety via the side chain of Tyr, Lys, hLys, Orn, Dap or Dab.

Preferably, the peptide is represented by any one of the formulae (10a) to (10v), (10b′) and (10g′).

In a preferred embodiment, the peptide (V) is represented by any one of the formulae (10a), (10b), (10b′) (10c), (10e), (10f), (10g), (10g′) (10h), (10i), (10j), (10k), (10m), (10n), (10p), (10q), (10s), (10t) and (10u), more preferably by any one of the formulae (10e), (10f), (10g), (10h), (10i), (10j), (10k), (10t) and (10u), and even more preferably by any one of the formulae (10e), (10f), (10g), (10h), (10i), (10j), and (10k). Most preferably, the peptide is represented by one of formulae (10f), (10g), (10j) and (10k).

In one embodiment, in the formulae (7a), (9a), and (10a) to (10k′) above at least one of Z₁ and Z₂ is a C₂₋₃ polyalkylene oxide-containing group, preferably a polyethylene oxide-containing group, which preferably comprises from 4 to 600, more preferably from 10 to 200, even more preferably from 15 to 80 repeating units.

The covalent attachment of a C₂₋₃ polyalkylene oxide-containing group, e.g., a polyethylene oxide-containing group, to the N- and/or C-terminus of the peptide (V) is advantageous as it significantly enhances the water solubility of the reactive conjugate and prevents unspecific lipophilic interaction between the reactive conjugate and the antibody during the conjugation reaction. This enables to achieve higher selectivity and DAR values and/or to use a broader range of payloads, in particular hydrophobic payloads such as vedotin (MMAE) or DM4.

In one embodiment, in the formulae (7a), (9a), and (10a) to (10k′) above, Z₁ is represented by the following formula (13a):

X⁸—(OCH₂CH₂)ₙ₇—X⁹— (13a)
wherein
X⁸ represents an alkyl group having 1 to 6 carbon atoms, such as a methyl or an ethyl group, or an amino-containing group, preferably a group of formula —(CH₂)ₙ₈—N(H)(R), in which R is selected from a hydrogen atom, an alkyl group, a cycloalkyl group and a carbonyl-containing group, such as an acetyl group, and n8 is an integer of 1 to 6, preferably 2;
X⁹ represents —(C═X)—, X being selected from O and S, preferably O; and n7 is an integer of 4 to 100, preferably 10 to 80, more preferably 15 to 40, most preferably 20 or 24; and/or
Z₂ is represented by the following formula (13b):

—X¹⁰—(CH₂CH₂O)ₙ₉—X¹¹ (13b)
wherein
n9 is an integer of 4 to 100, preferably 10 to 80, more preferably 15 to 40;
X¹⁰ is a single covalent bond, NH, O or S; preferably NH;
X¹¹ represents an alkyl group having 1 to 6 carbon atoms such as a methyl group, a carbonyl-containing group such an acetyl group or a group of formula —(CH₂)ₙ₁₀—CO₂H, a thiocarbonyl-containing group, a group of formula —(CH₂)ₙ₁₀OR, a group of formula —(CH₂)ₙ₁₀—SO₃H, or an amino-containing group such as a group of formula —(CH₂)ₙ₁₀—(C═X)—N(R′)(R) or —(CH₂)ₙ₁₀—N(R′)(R), in which X is O or S, R and R′ are each independently selected from a hydrogen atom, an alkyl group or a cycloalkyl group, and n10 is an integer of 1 to 6; X¹¹ being preferably a methyl group, or a group represented by the following formula (13b′):

—(CH₂)ₙ₁₁—(C═X)N(R)—(CH₂)ₙ₁₂—(C═X)N(H)(R) (13b′)
wherein
each X is independently selected from O and S, preferably O;
each R is independently selected from a hydrogen atom, an alkyl group and a cycloalkyl group; and
n11 and n12 are each independently an integer of 1 to 6, preferably 1 or 2, more preferably 2;
X¹¹ being most preferably a methyl group.

In one embodiment, in the above formulae (7a), (9a), and (10a) to (10k′), Z₁ represents a polyethylene oxide-containing group comprising from 10 to 200, preferably from 15 to 80 polyethylene oxide repeating units, more preferably a group of formula (13a) as defined above, and Z₂ is a group represented by N(H)(R) wherein R represents a hydrogen atom, an alkyl group, or a cycloalkyl group. Preferably, the peptide (V) is represented by any one of the formulae (10e), (10f), (10g), (10h), (10i), (10j), (10k), (10t) and (10u), more preferably by any one of the formulae (10e), (10f), (10g), (10h), (10i), (10j), and (10k), and even more preferably by formula (10f).

In a more preferred embodiment, the peptide is represented by one of the following formulae (14a) to (14k′):

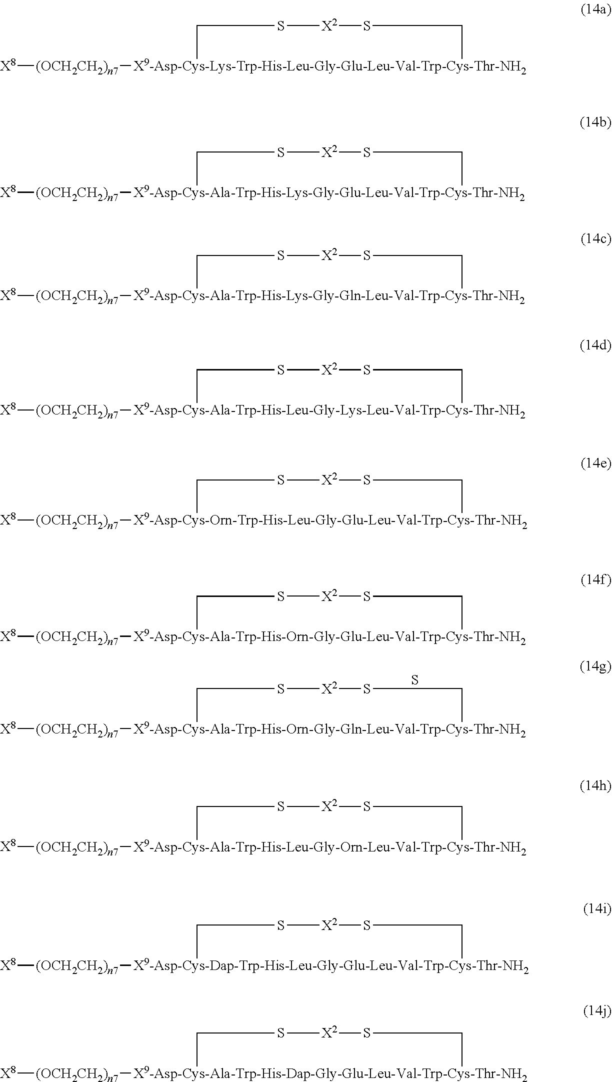

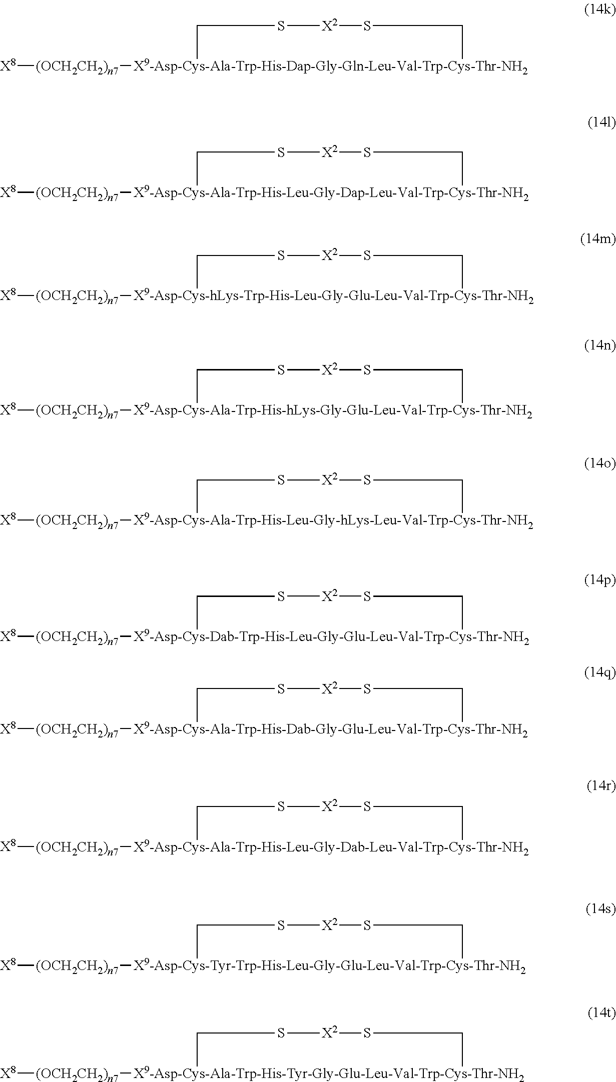

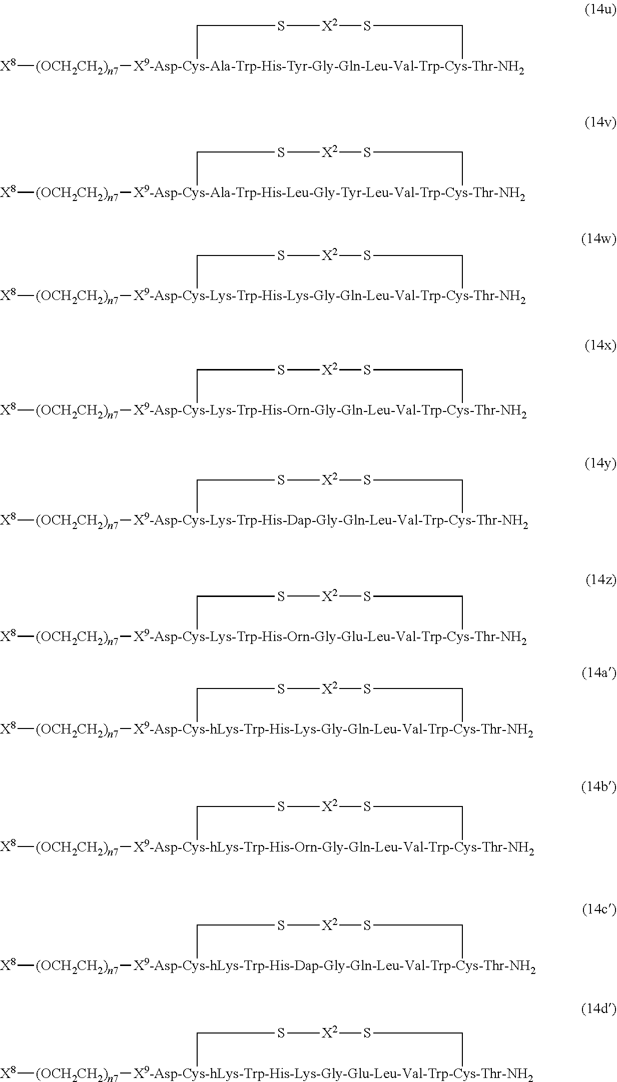

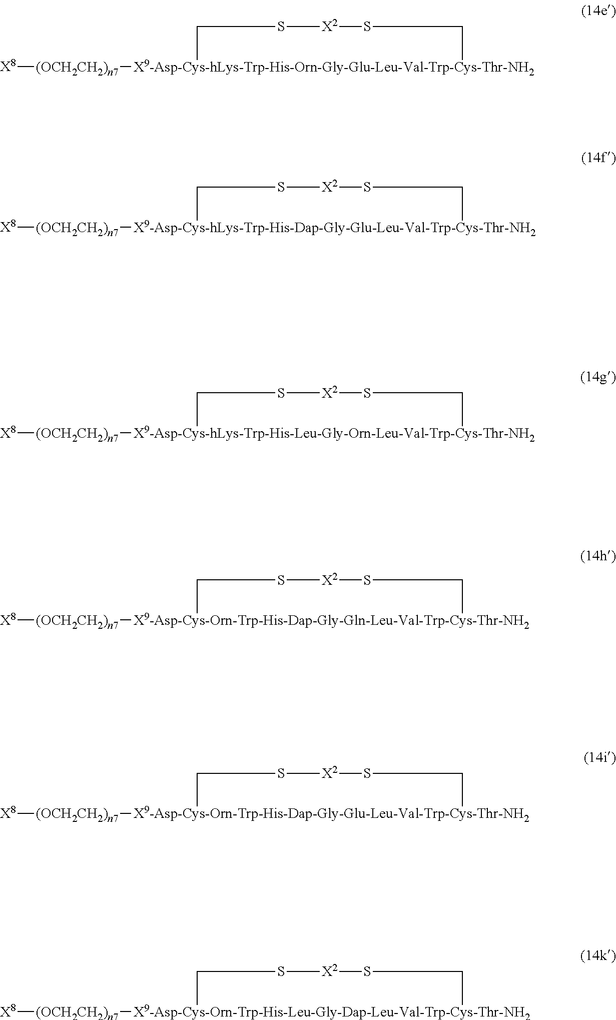

In the formulae (14a) to (14k′) above, X² is as described above with respect to formula (7a), X⁸, X⁹, and n7 are as described above with respect to formula (13a). Preferably, X⁸ is a methyl group or a group represented by the formula —(CH₂)ₙ₈—NH₂ with n8=1 to 6, X⁹ is —(C═O)—, and n7 is an integer of 15 to 40, such as 20 or 24. In these formulae, the peptide is covalently attached to the reactive moiety via the side chain of Tyr, Lys, hLys, Orn, Dap or Dab.

Preferably, the peptide is represented by any one of the formulae (14a) to (14v), (14b′) and (14g′).

In a preferred embodiment, the peptide (V) is represented by any one of the formulae (14a), (14b), (14b′) (14c), (14e), (14f), (14g), (14g′) (14h), (14i), (14j), (14k), (14m), (14n), (14p), (14q), (14s), (14t) and (14u), more preferably by any one of the formulae (14e), (14f), (14g), (14h), (14i), (14j), (14k), (14t) and (14u), and even more preferably by any one of the formulae (14e), (14f), (14g), (14h), (14i), (14j), and (14k). Most preferably, the peptide is represented by one of formulae (14f), (14g), (14j) and (14k).

In an embodiment, the disulfide bridge(s) between cysteine residues in the above formulae (i.e. the disulfide bridges of formula —(S—X²—S)— or —(S—X³—S)—) can each independently be replaced by a divalent group suitable for side-chain-to-side-chain cyclization (sometimes called “cysteine re-bridging”; see e.g. Stefanucci et al. Scientific Reports 2019, 9:5771). Examples of suitable divalent groups include divalent xylene groups, divalent maleimide groups, divalent triazole-containing groups, divalent carbonyl-containing groups (e.g. a divalent acetone group), divalent succinimide groups (which can be obtained by reacting the cysteine side chains with e.g. an aryloxymaleimide reagent; see Marculescu et al. Chem. Commun. 2014, 50, 7139), divalent thioether groups (which can be obtained by reacting the cysteine side chains with e.g. a bis-sulfone or an allyl sulfone reagent; see Brocchini et al. Nat. Protoc. 2006, 1, 2241-2252), and divalent pyridazinedione groups (which can be obtained by reacting the cysteine side chains with e.g. a dibromopyridazinedione reagent; see Chudamasa et al. Chem. Commun. 2011, 47, 8781-8783). In particular, the disulfide bridge(s) can each independently be replaced by a divalent triazole-containing group, which may be obtained by “click” chemistry. In this case, the cysteine residues (forming a bridge in the above formulae) can be replaced by amino acids having a side chain containing a functional group suitable for click chemistry, i.e. an alkyne group or an azido group, which can be reacted to from a divalent triazole moiety (e.g. a 1,4-disubstituted-1,2,3-triazole moiety).

According to one embodiment, the compound of the present invention is a compound represented by the formula V—(O—(C═O)—O—P¹)ₙ, V—(O—(C═O)—P¹)ₙ, V—(S—(C═O)—P¹)ₙ, V—(S—(C═O)—O—P¹)ₙ, V—(O—(C═S)—O—P¹)ₙ, V—(O—(C═O)—S—P¹)ₙ, V—(S—(C═O)—S—P¹)ₙ, V—(S—(C═S)—O—P¹)ₙ, V—(O—(C═S)—S—P¹)ₙ, V—(S—(C═S)—P¹)ₙ, V—(O—(C═O)—NH—P¹)ₙ, V—(S—(C═S)—S—P¹)ₙ, V—(O—(C═O)—O-L-P¹)ₙ, V—(O—(C═O)-L-P¹)ₙ, V—(S—(C═O)-L-P¹)ₙ, V—(S—(C═O)—O-L-P¹)ₙ, V—(O—(C═S)—O-L-P¹)ₙ, V—(O—(C═O)—S-L-P¹)ₙ, V—(S—(C═O)—S-L-P¹)ₙ V—(S—(C═S)—O-L-P¹)ₙ, V—(O—(C═S)—S-L-P¹)ₙ, V—(S—(C═S)-L-P¹)ₙ, V—(O—(C═O)—NH-L-P¹)ₙ, V—(S—(C═S)—S-L-P¹)ₙ, V—(O—(C═O)—O—K(-L-P¹)ₙ′)ₙ, V—(O—(C═O)—K(-L-P¹)ₙ′)ₙ, V—(S—(C═O)—K(-L-P¹)ₙ′)ₙ, V—(S—(C═O)—O—K(-L-P¹)ₙ′)ₙ, V—(—(C═S)—O—K(-L-P¹)ₙ′)ₙ, V—(O—(C═O)—S—K(-L-P¹)ₙ′)ₙ, V—(S—(C═O)—S—K(-L-P¹)ₙ′)ₙ, V—(S—(C═S)—O—K(-L-P¹)ₙ′)ₙ, V—(O—(C═S)—S—K(-L-P¹)ₙ′)ₙ, V—(S—(C═S)—K(-L-P¹)ₙ′)ₙ, V—(O—(C═O)—NH—K(-L-P¹)ₙ′)ₙ, V—(S—(C═S)—S—K(-L-P¹)ₙ′)ₙ, V—(O—(C═O)—O—K(—P¹)ₙ′)ₙ, V—(O—(C═O)—K(—P¹)ₙ′)ₙ, V—(S—(C═O)—K(—P¹)ₙ′)ₙ, V—(S—(C═O)—O—K(—P¹)ₙ′)ₙ, V—(O—(C═S)—O—K(—P¹)ₙ′)ₙ, V—(O—(C═O)—S—K(—P¹)ₙ′)ₙ, V—(S—(C═O)—S—K(—P¹)ₙ′)ₙ, V—(S—(C═S)—O—K(—P¹)ₙ′)ₙ, V—(O—(C═S)—S—K(—P¹)ₙ′)ₙ, V—(S—(C═S)—K(—P¹)ₙ′)ₙ, V—(O—(C═O)—NH—K(—P¹)ₙ′)ₙ, V—(S—(C═S)—S—K(—P¹)ₙ′)ₙ V-(M-O—(C═O)—O—P¹)ₙ, V-(M-O—(C═O)—P¹)ₙ, V-(M-S—(C═O)—P¹)ₙ, V-(M-S—(C═O)—O—P¹)ₙ, V-(M-O—(C═S)—O—P¹)ₙ, V-(M-O—(C═O)—S—P¹)ₙ, V-(M-S—(C═O)—S—P¹)ₙ, V-(M-S—(C═S)—O—P¹)ₙ, V-(M-O—(C═S)—S—P¹)ₙ, V-(M-S—(C═S)—P¹)ₙ, V-(M-O—(C═O)—NH—P¹)ₙ V-(M-S—(C═S)—S—P¹)ₙ, V-(M-O—(C═O)—O-L-P¹)ₙ, V-(M-O—(C═O)-L-P¹)ₙ, V-(M-S—(C═O)-L-P¹)ₙ, V-(M-S—(C═O)—O-L-P¹)ₙ, V-(M-O—(C═S)—O-L-P¹)nV-(M-O—(C═O)—S-L-P¹)ₙ, V-(M-S—(C═O)—S-L-P¹)ₙ, V-(M-S—(C═S)—O-L-P¹)ₙ, V-(M-O—(C═S)—S-L-P¹)ₙ, V-(M-S—(C═S)-L-P¹)ₙ, V-(M-O—(C═O)—NH-L-P¹)ₙ, V-(M-S—(C═S)—S-L-P¹)ₙ, V-(M-O—(C═O)—O—K(-L-P¹)ₙ′)ₙ, V-(M-O—(C═O)—K(-L-P¹)ₙ′)ₙ, V-(M-S—(C═O)—K(-L-P¹)ₙ′)ₙ, V-(M-S—(C═O)—O—K(-L-P¹)ₙ′)ₙ, V-(M-O—(C═S)—O—K(-L-P¹)ₙ′)ₙ, V-(M-O—(C═O)—S—K(-L-P¹)ₙ′)ₙ, V-(M-S—(C═O)—S—K(-L-P¹)ₙ′)ₙ, V-(M-S—(C═S)—O—K(-L-P¹)ₙ′)ₙ, V-(M-O—(C═S)—S—K(-L-P¹)ₙ′)ₙ, V-(M-S—(C═S)—K(-L-P¹)ₙ′)ₙ, V-(M-O—(C═O)—NH—K(-L-P¹)ₙ′)ₙ, V-(M-S—(C═S)—S—K(-L-P¹)ₙ′)ₙ, V-(M-O—(C═O)—O—K(—P¹)ₙ′)ₙ, V-(M-O—(C═O)—K(—P¹)ₙ′)ₙ, V-(M-S—(C═O)—K(—P¹)ₙ′)ₙ, V-(M-S—(C═O)—O—K(—P¹)ₙ′)ₙ, V-(M-O—(C═S)—O—K(—P¹)ₙ′)ₙ, V-(M-O—(C═O)—S—K(—P¹)ₙ′)ₙ, V-(M-S—(C═O)—S—K(—P¹)ₙ′)ₙ, V-(M-S—(C═S)—O—K(—P¹)ₙ′)ₙ, V-(M-O—(C═S)—S—K(—P¹)ₙ′)ₙ, V-(M-S—(C═S)—K(—P¹)ₙ′)ₙ, V-(M-O—(C═O)—NH—K(—P¹)ₙ′)ₙ or V-(M-S—(C═S)—S—K(—P¹)ₙ′)ₙ, wherein V, P¹, L, K, M, n and n′ are as defined above, and wherein preferably at least one—e.g. two, three, four, or more than four—of V, P¹, L, K, M, n and n′ is/are defined as follows:
(α) V is a peptide of formula (10e), (10f), (14e) or (14f), most preferably (10f) or (14f);
(β) P¹ is a moiety derived from NOTA, DOTA, NODAGA, DTPA, N3, TZ, TCO, DBCO, BCN, auristatin (e.g. MMAE), DM4 and PNU-159582;
(γ) L is a linker selected from
   (a1) an alkylene group having 2 to 6 carbons (—(CH₂)₂₋₆—),
   (b1) a polyalkylene group of formula —NH—(CH₂CH₂O)ₙ₁—CH₂CH₂—, n1 being an integer of 0-35, and
   (c1) a peptidic linker comprising 2 to 12 amino acids, which is optionally cleavable, preferably a cleavable peptidic linker comprising a Val-Cit unit, a Val-Ala unit, a Val-Cit-PABC or a Val-Cit-PABC-DMEA unit;
(δ) K is a branching group of formula (3d) or (3e), wherein preferably m1 is 1 and/or m2 is 1;
(ε) M is a group of formula (5a) or (5l), preferably (5a);
(ζ) n is 1; and
(η) n′ is 2.

According to a preferred embodiment, in the above formulae, all of V, K, M, n and n′ are defined as follows while one or both of P¹ and L are preferably as defined above under items (β) and (γ):
(α) V is a peptide of formula (10e), (10f) or (14f), most preferably (10f) or (14f);
(δ) K is a branching group of formula (3d) or (3e), wherein m1 is 1 and/or m2 is 1;
(ε) M is a group of formula (5a) or (5l), preferably (5a);
(ζ) n is 1; and
(η) n′ is 2.

If (β) P¹ is a moiety derived from auristatin, e.g. MMAE, (γ) L represents preferably a cleavable linker comprising a Val-Cit unit, a Val-Ala unit or a Val-Cit-PABC unit, more preferably a Val-Cit-PABC unit. If (β) P¹ is a moiety derived from PNU-159582, (γ) L represents preferably a cleavable linker comprising a Val-Cit-PABC-DMEA unit.

According to one embodiment, the compound of the present invention is a compound represented by the formula V—(O—(C═O)—O—P¹)ₙ, V—(O—(C═O)—P¹)ₙ, V—(S—(C═O)—P¹)ₙ, V—(S—(C═O)—O—P¹)ₙ, V—(O—(C═S)—O—P¹)ₙ, V—(O—(C═O)—S—P¹)ₙ, V—(S—(C═O)—S—P¹)ₙ, V—(S—(C═S)—O—P¹)ₙ, V—(O—(C═S)—S—P¹)ₙ, V—(S—(C═S)—P¹)ₙ, V—(O—(C═O)—NH—P¹)ₙ, V—(S—(C═S)—S—P¹)ₙ, V—(O—(C═O)—O—K(—P¹)ₙ′)ₙ, V—(O—(C═O)—K(—P¹)ₙ′)ₙ, V—(S—(C═O)—K(—P¹)ₙ′)ₙ, V—(S—(C═S)—O—K(—P¹)ₙ′)ₙ, V—(O—(C═S)—O—K(—P¹)ₙ′)ₙ, V—(O—(C═O)—S—K(—P¹)ₙ′)ₙ, V—(S—(C═O)—S—K(—P¹)ₙ′)ₙ, V—(S—(C═S)—O—K(—P¹)ₙ′)ₙ, V—(O—(C═S)—S—K(—P¹)ₙ′)ₙ, V—(S—(C═S)—K(—P¹)ₙ′)ₙ, V—(O—(C═O)—NH—K(—P¹)ₙ′)ₙ, V—(S—(C═S)—S—K(—P¹)ₙ′)ₙ, V-(M-O—(C═O)—O—P¹)ₙ, V-(M-O—(C═O)—P¹)ₙ, V-(M-S—(C═O)—P¹)ₙ, V-(M-S—(C═O)—O—P¹)ₙ, V-(M-O—(C═S)—O—P¹)ₙ, V-(M-O—(C═O)—S—P¹)ₙ, V-(M-S—(C═O)—S—P¹)ₙ, V-(M-S—(C═S)—O—P¹)ₙ, V-(M-O—(C═S)—S—P¹)ₙ, V-(M-S—(C═S)—P¹)ₙ, V-(M-O—(C═O)—NH—P¹)ₙ, V-(M-S—(C═S)—S—P¹)ₙ, V-(M-O—(C═O)—O—K(—P¹)ₙ′)ₙ, V-(M-O—(C═O)—K(—P¹)ₙ′)ₙ, V-(M-S—(C═O)—K(—P¹)ₙ′)ₙ, V-(M-S—(C═O)—O—K(—P¹)ₙ′)ₙ, V-(M-O—(C═S)—O—K(—P¹)ₙ′)ₙ, V-(M-O—(C═O)—S—K(—P¹)ₙ′)ₙ, V-(M-S—(C═O)—S—K(—P¹)ₙ′)ₙ, V-(M-S—(C═S)—O—K(—P¹)ₙ′)ₙ, V-(M-O—(C═S)—S—K(—P¹)ₙ′)ₙ, V-(M-S—(C═S)—K(—P¹)ₙ′)ₙ, V-(M-O—(C═O)—NH—K(—P¹)ₙ′)ₙ or V-(M-S—(C═S)—S—K(—P¹)ₙ′)ₙ, wherein V, P¹, K, M, n and n′ are as defined above, and preferably wherein at least one—e.g. two, three, four, or more than four—of V, P¹, K, M, n and n′ is/are defined as follows:
(α) V is a peptide of formula (10e), (10f) or (14f), most preferably (10f) or (14f);
(β) P¹ is a moiety derived from NOTA, DOTA, NODAGA, DTPA, N3, TZ, TCO, DBCO, and BCN;
(δ) K is a branching group of formula (3d) or (3e), wherein preferably m1 is 1 and/or m2 is 1;
(ε) M is a group of formula (5a) or (5l), preferably (5a);
(ζ) n is 1; and
(η) n′ is 2.

According to a preferred embodiment, in the above formulae, all of V, K, M, n and n′ are defined as follows while P¹ is preferably as defined above under item (β):
(α) V is a peptide of formula (10e), (10f) or (14f), most preferably (10f) or (14f);
(δ) K is a branching group of formula (3d) or (3e), wherein m1 is 1 and/or m2 is 1;
(ε) M is a group of formula (5a) or (5l), preferably (5a);
(ζ) n is 1; and
(η) n′ is 2.

According to one embodiment, the compound of the present invention is a compound represented by the formula V-O-(C═O)—O—P¹, V-O-(C═O)—P¹, V—(S—(C═O)—P¹, V-S-(C═O)—O—P¹, V-O-(C═S)—O—P¹, V-O-(C═O)—S—P¹, V—(S—(C═O)—S—P¹, V—(S—(C═S)—O—P¹, V-O-(C═S)—S—P¹, V—(S—(C═S)—P¹, V-O-(C═O)—NH—P¹, V—(S—(C═S)—S—P¹, V-O-(C═O)—O—K(—P¹)₂, V-O-(C═O)—K(—P¹)₂, V—(S—(C═O)—K(—P¹)₂, V—(S—(C═O)—O—K(—P¹)₂, V-O-(C═S)—O—K(—P¹)₂, V-O-(C═O)—S—K(—P¹)₂, V—(S—(C═O)—S—K(—P¹)₂, V—(S—(C═S)—O—K(—P¹)₂, V-O-(C═S)—S—K(—P¹)₂, V—(S—(C═S)—K(—P¹)₂, V-O-(C═O)—NH—K(—P¹)₂, V—(S—(C═S)—S—K(—P¹)₂, V-M-O—(C═O)—O—P¹, V-M-O—(C═O)—P¹, V-M-S—(C═O)—P¹, V-M-S—(C═O)—O—P¹, V-M-O—(C═S)—O—P¹, V-M-O—(C═O)—S—P¹, V-M-S—(C═O)—S—P¹, V-M-S—(C═S)—O—P¹, V-M-O—(C═S)—S—P¹, V-M-S—(C═S)—P¹, V-M-O—(C═O)—NH—P¹, V-M-S—(C═S)—S—P¹, V-M-O—(C═O)—O—K(—P¹)₂, V-M-O—(C═O)—K(—P¹)₂, V-M-S—(C═O)—K(—P¹)₂, V-M-S—(C═O)—O—K(—P¹)₂, V-M-O—(C═S)—O—K(—P¹)₂, V-M-O—(C═O)—S—K(—P¹)₂, V-M-S—(C═O)—S—K(—P¹)₂, V-M-S—(C═S)—O—K(—P¹)₂, V-M-O—(C═S)—S—K(—P¹)₂, V-M-S—(C═S)—K(—P¹)₂, V-M-O—(C═O)—NH—K(—P¹)₂ or V-M-S—(C═S)—S—K(—P¹)₂, wherein V, P¹, K and M are as defined above, and preferably wherein at least one—e.g. two, three, or four—of V, P¹, K and M is/are defined as follows:
(α) V is a peptide of formula (10e), (10f) or (14f), most preferably (10f) or (14f);
(β) P¹ is a moiety derived from NOTA, DOTA, NODAGA, DTPA, N3, TZ, TCO, DBCO, and BCN;
(δ) K is a branching group of formula (3d) or (3e), wherein preferably m1 is 1 and/or m2 is 1; and
(ε) M is a group of formula (5a) or (5l), preferably (5a).

According to a preferred embodiment, in the above formulae, all of V, K and M are defined as follows while P¹ is preferably as defined above under item (β):
(α) V is a peptide of formula (10e), (10f) or (14f), most preferably (10f) or (14f);
(δ) K is a branching group of formula (3d) or (3e), wherein m1 is 1 and/or m2 is 1; and
(ε) M is a group of formula (5a) or (5l), preferably (5a).

According to one embodiment, the compound of the present invention is a compound represented by the formula V—(O—(C═O)—O—(CH₂)₂₋₆—P¹)ₙ, V—(O—(C═O)—(CH₂)₂₋₆—P¹)ₙ, V—(S—(C═O)—(CH₂)₂₋₆—P¹)ₙ, V—(S—(C═O)—O—(CH₂)₂₋₆—P¹)ₙ, V—(O—(C═S)—O—(CH₂)₂₋₆—P¹)ₙ, V—(O—(C═O)—S—(CH₂)₂₋₆—P¹)ₙ, V—(S—(C═O)—S—(CH₂)₂₋₆—P¹)ₙ, V—(S—(C═S)—O—(CH₂)₂₋₆—P¹)ₙ, V—(O—(C═S)—S—(CH₂)₂₋₆—P¹)ₙ, V—(S—(C═S)— (CH₂)₂₋₆—P¹)ₙ, V—(O—(C═O)—NH—(CH₂)₂₋₆—P¹)ₙ, V—(S—(C═S)—S—(CH₂)₂₋₆—P¹)ₙ, V—(O—(C═O)—O—K(—(CH₂)₂₋₆—P¹)ₙ′)ₙ, V—(O—(C═O)—K(—(CH₂)₂₋₆—P¹)ₙ′)ₙ, V—(S—C═O)—K(—(CH₂)₂₋₆—P¹)ₙ′)ₙ, V—(S—(C═O)—O—K(—(CH₂)₂₋₆—P¹)ₙ′)ₙ, V—(O—(C═S)—O—K(—(CH₂)₂₋₆—P¹)ₙ′)ₙ, V—(O—(C═O)—S—K(—(CH₂)₂₋₆—P¹)ₙ′)ₙ, V—(S—(C═O)—S—K(—(CH₂)₂₋₆—P¹)ₙ′)ₙ, V—(S—(C═S)—O—K(—(CH₂)₂₋₆—P¹)ₙ′)ₙ, V—(O—(C═S)—S—K(—(CH₂)₂₋₆—P¹)ₙ′)ₙ, V—(S—(C═S)—K(—(CH₂)₂₋₆—P¹)ₙ′)ₙ, V(O—(C═O)—NH—K(—(CH₂)₂₋₆—P¹)ₙ′)ₙ, V—(S—(C═S)—S—K(—(CH₂)₂₋₆—P¹)ₙ′)ₙ, V-(M-O—(C═O)—O—(CH₂)₂₋₆—P¹)ₙ, V O-M-O(C═O)— (CH₂)₂₋₆—P¹)ₙ, V-(M-S—(C═O)—(CH₂)₂₋₆—P¹)ₙ, V-(M-S—(C═O)—O—(CH₂)₂₋₆—P¹)ₙ, V—O-M-(C═S)—O—(CH₂)₂₋₆—P¹)ₙ, V-(M-O—(C═O)—S—(CH₂)₂₋₆—P¹)ₙ, V-(M-S—(C═)—S—(CH₂)₂₋₆—P¹)ₙ, V-(M-S—(C═S)—O—(CH₂)₂₋₆—P¹)ₙ, V-(M-O—(C═S)—S—(CH₂)₂₋₆—P¹)ₙ, V-(M-S—(C═S)—(CH₂)₂₋₆—P¹)ₙ, V-(M-O—(C═O)—NH—(CH₂)₂₋₆—P¹)ₙ, V-(M-S—(C═S)—S—(CH₂)₂₋₆—P¹)ₙ, V-(M-O—(C═O)—O—K(—(CH₂)₂₋₆—P¹)ₙ′)ₙ, V-(M-O—(C═O)—K(—(CH₂)₂₋₆—P¹)ₙ′)ₙ, V-(M-S—(C═O)—K(—(CH₂)₂₋₆—P¹)ₙ′)ₙ, V-(M-S—(C═)—O—K(—(CH₂)₂₋₆—P¹)ₙ′)ₙ, V—(—(C═S)—O—K(—(CH₂)₂₋₆—P¹)ₙ′)ₙ, V-(M-O—(C═O)—S—K(—(CH₂)₂₋₆—P¹)ₙ′)ₙ, V-(M-S—(C═O)—S—K(—(CH₂)₂₋₆—P¹)ₙ′)ₙ, V-(M-S—(C═S)—O—K(—(CH₂)₂₋₆—P¹)ₙ′)ₙ, V-(M-O—(C═S)—S—K(—(CH₂)₂₋₆—P¹)ₙ′)ₙ, V-(M-S—(C═S)—K(—(CH₂)₂₋₆—P¹)ₙ′)ₙ, V-(M-O—(C═O)—NH—K(—(CH₂)₂₋₆—P¹)ₙ′)ₙ, V-(M-S—(C═S)—S—K(—(CH₂)₂₋₆—P¹)_{n′})ₙ, V—(O(C═O)—O—NH—(CH₂CH₂O)ₙ₁—CH₂CH₂—P¹)ₙ, V—(O—(C═O)—NH—(CH₂CH₂O)ₙ₁—CH₂CH₂—P¹)ₙ, V—(S—(C═O)—NH—(CH₂CH₂O)ₙ₁—CH₂CH₂—P¹)ₙ, V—(S—(C═O)—O—NH—(CH₂CH₂O)ₙ₁—CH₂CH₂—P¹⁾n, V—(S—(C═S)—O—NH—(CH₂CH₂O)ₙ₁—CH₂CH₂—P¹)ₙ, V—(O—(C═O)—S—NH—(CH₂CH₂O)ₙ₁—CH₂CH₂—P¹)ₙ, V—(S—(C═O)—S—NH—(CH₂CH₂O)ₙ₁—CH₂CH₂—P¹)ₙ, V—(S—(C═S)—O—NH—(CH₂CH₂O)ₙ₁—CH₂CH₂—P¹)ₙ, V—(O—(C═S)—S—NH—(CH₂CH₂O)ₙ₁—CH₂CH₂—P¹)ₙ, V—(S—(C═S)—NH—(CH₂CH₂O)ₙ₁—CH₂CH₂—P¹)ₙ, V—(O—(C═O)—NH—NH—(CH₂CH₂O)ₙ₁—CH₂CH₂—P¹)ₙ, V—(S—(C═S)—S—NH—(CH₂CH₂O)ₙ₁—CH₂CH₂—P¹)ₙ, V—(O—(C═)—O—K(—NH—(CH₂CH₂O)ₙ₁—CH₂CH₂—P¹)ₙ′)ₙ, V—(O—(C═O)—K(—NH—(CH₂CH₂O)ₙ₁—CH₂CH₂—P¹)ₙ′)ₙ, V—(S—(C═O)—K(—NH—(CH₂CH₂O)ₙ₁—CH₂CH₂—P¹)ₙ′)ₙ, V—(S—(C═O)—O—K(—NH—(CH₂CH₂O)ₙ₁—CH₂CH₂—P¹)ₙ′)ₙ, V—(O—(C═S)—O—K(—NH—(CH₂CH₂O)ₙ₁—CH₂CH₂—P¹)ₙ′)ₙ, V—(O—(C═O)—S—K(—NH—(CH₂CH₂O)ₙ₁—CH₂CH₂—P¹)ₙ′)ₙ, V—(S—(C═O)—S—K(—NH—(CH₂CH₂O)ₙ₁—CH₂CH₂—P¹)ₙ′)ₙ, V—(S—(C═S)—O—K(—NH—(CH₂CH₂O)ₙ₁—CH₂CH₂—P¹)ₙ′)ₙ, V—(O—(C═S)—S—K(—NH—(CH₂CH₂O)ₙ₁—CH₂CH₂—P¹)ₙ′)ₙ, V—(S—(C═S)—K(—NH—(CH₂CH₂O)ₙ₁—CH₂CH₂—P¹)ₙ′)ₙ, V—(O—(C═O)—NH—K(—NH—(CH₂CH₂O)ₙ₁—CH₂CH₂—P¹)ₙ′)ₙ, V—(S—(C═S)—S—K(—NH—(CH₂CH₂O)ₙ₁—CH₂CH₂—P¹)ₙ′)ₙ, V-(M-O—(C═O)—O—NH—(CH₂CH₂O)ₙ₁—CH₂CH₂—P¹)ₙ, V-(M-O—(C═O)—NH—(CH₂CH₂O)ₙ₁—CH₂CH₂—P¹)ₙ, V-(M-S—(C═O)—NH—(CH₂CH₂O)ₙ₁—CH₂CH₂—P¹)ₙ, V-(M-S—(C═O)—O—NH—(CH₂CH₂O)ₙ₁—CH₂CH₂—P¹)ₙ, V-(M-O—(C═S)—O—NH—(CH₂CH₂O)ₙ₁—CH₂CH₂—P¹)ₙ, V-(M-O—(C═O)—S—NH—(CH₂CH₂O)ₙ₁—CH₂CH₂—P¹)ₙ, V-(M-S—(C═O)—S—NH—(CH₂CH₂O)ₙ₁—CH₂CH₂—P¹)ₙ, V-(M-S—(C═S)—O—NH—(CH₂CH₂O)ₙ₁—CH₂CH₂—P¹)ₙ, V-(M-O—(C═S)—S—NH—(CH₂CH₂O)ₙ₁—CH₂CH₂—P¹)ₙ, V-(M-S—(C═S)—NH—(CH₂CH₂O)ₙ₁—CH₂CH₂—P¹)ₙ, V-(M-O—(C═O)—NH—NH—(CH₂CH₂O)ₙ₁—CH₂CH₂—P¹)ₙ, V-(M-S—(C═S)—S—NH—(CH₂CH₂O)ₙ₁—CH₂CH₂—P¹)ₙ, V-(M-O—(C═O)—O—K(—NH—(CH₂CH₂O)ₙ₁—CH₂CH₂—P¹)ₙ′)ₙ, V-(M-O—(C═O)—K(—NH—(CH₂CH₂O)ₙ₁—CH₂CH₂—P¹)ₙ′)ₙ, V-(M-S—(C═O)—K(—NH—(CH₂CH₂O)ₙ₁—CH₂CH₂—P¹)ₙ′)ₙ, V-(M-S—(C═O)—O—K(—NH—(CH₂CH₂O)ₙ₁—CH₂CH₂—P¹)ₙ′)ₙ, V-(M-O—(C═S)—O—K(—NH—(CH₂CH₂O)ₙ₁—CH₂CH₂—P¹)ₙ′)ₙ, V- (M-O—(C═O)—S—K(—NH—(CH₂CH₂O)ₙ₁—CH₂CH₂—P¹)ₙ′)ₙ, V-(M-S—(C═O)—S—K(—NH—(CH₂CH₂O)ₙ₁—CH₂CH₂—P¹)ₙ′)ₙ, V-(M-S—(C═S)—O—K(—NH—(CH₂CH₂O)ₙ₁—CH₂CH₂—P¹)ₙ′)ₙ, V-(M-O—(C═S)—S—K(—NH—(CH₂CH₂O)ₙ₁—CH₂CH₂—P¹)ₙ′)ₙ, V-(M-S—(C═S)—K(—NH—(CH₂CH₂O)ₙ₁—CH₂CH₂—P¹)ₙ′)ₙ, V-(M-O—(C═O)—NH—K(—NH—(CH₂CH₂O)ₙ₁—CH₂CH₂—P¹)ₙ′)ₙ, V-(M-S—(C═S)—S—K(—NH—(CH₂CH₂O)ₙ₁—CH₂CH₂—P¹)ₙ′)ₙ, V—(O—(C═O)—O-AA₂₋₁₂-P¹)ₙ, V—(O—(C═O)-AA₂₋₁₂-P¹)ₙ, V—(C═O)-AA₂₋₁₂-P¹)ₙ, V—(S—(C═O)—O-AA₂₋₁₂—P¹)ₙ, V—(O—(C═S)—O-AA₂₋₁₂—P¹)ₙ, V—(O—(C═O)—S-AA₂₋₁₂—P¹)ₙ, V—(S—(C═O)—S-AA₂₋₁₂-P¹)ₙ, V—(S—(C═S)—O-AA₂₋₁₂-P1)ₙ, V—(O—(C═S)—S-AA₂₋₁₂—P¹)ₙ, V—(S—(C═S)-AA₂₋₁₂—P¹)ₙ, V—(O—(C═O)—NH-AA₂₋₁₂—P¹)ₙ, V—(S—(C═S)—S-AA₂₋₁₂—P¹)ₙ, V—(O—(C═O)—O—K(-AA₂₋₁₂—P¹)ₙ′)ₙ, V—(O—(C═O)—K(-AA₂₋₁₂—P¹)ₙ′)ₙ, V—(S—(C═O)—K(-AA₂₋₁₂—P¹)ₙ′)ₙ, V—(S—(C═O)—O—K(-AA₂₋₁₂—P¹)ₙ′)ₙ, V—(O—(C═S)—O—K(-AA₂₋₁₂-P¹)ₙ′)ₙ, V—(O—(C═O)—S—K(-AA₂₋₁₂-P¹)ₙ′)ₙ, V—(S—(C═O)—S—K(-AA₂₋₁₂-P¹)ₙ′)ₙ, V—(S—(C═S)—O—K(-AA₂₋₁₂—P¹)ₙ′)ₙ, V—(O—(C═S)—S—K(-AA₂₋₁₂-P¹)ₙ′)ₙ, V—(S—(C═S)—K(-AA₂₋₁₂-P¹)ₙ′)ₙ, V—(O—(C═O)—NH—K(-AA₂₋₁₂-P¹)ₙ′)ₙ, V—(S—(C═S)—S—K(-AA₂₋₁₂-P¹)ₙ′)ₙ, V-(M-O—(C═O)—O-AA₂₋₁₂-P¹)ₙ, V-(M-O—(C═O)-AA₂₋₁₂-P¹)ₙ, V-(M-S—(C═O)-AA₂₋₁₂-P¹)ₙ, V-(M-S—(C═O)—O-AA₂₋₁₂-P¹)ₙ, V-(M-O—(C═S)—O-AA₂₋₁₂-P¹)ₙ, V-(M-O—(C═O)—S-AA₂₋₁₂-P¹)ₙ, V-(M-S—(C═O)—S-AA₂₋₁₂-P¹)ₙ, V-(M-S—(C═S)—O-AA₂₋₁₂-P¹)ₙ, V-(M-O—(C═S)—S-AA₂₋₁₂-P¹)ₙ, V-(M-S—(C═S)-AA₂₋₁₂-P¹)ₙ, V-(M-O—(C═O)—NH-AA₂₋₁₂-P¹)ₙ, V-(M-S—(C═S)—S-AA₂₋₁₂-P¹)ₙ, V-(M-O—(C═O)—O—K(-AA₂₋₁₂-P¹)ₙ′)ₙ, V-(M-O—(C═O)—K(-AA₂₋₁₂—P¹)ₙ′)ₙ, V-(M-S—(C═O)—K(-AA₂₋₁₂-P¹)ₙ′)ₙ, V-(M-S—(C═O)—O—K(-AA₂₋₁₂-P¹)ₙ′)ₙ, V-(M-O—(C═S)—O—K(-AA₂₋₁₂-P¹)ₙ′)ₙ, V-(M-O—(C═O)—S—K(-AA₂₋₁₂-P¹)ₙ′)ₙ, V-(M- S—(C═O)—S—K(-AA₂₋₁₂-P¹)ₙ′)ₙ, V-(M-S—(C═S)—O—K(-AA₂₋₁₂-P¹)ₙ′)ₙ, V-(M-O—(C═S)—S—K(-AA₂₋₁₂-P¹)ₙ′)ₙ, V-(M-S—(C═S)—K(-AA₂₋₁₂-P¹)ₙ′)ₙ, V-(M-O—(C═O)—NH—K(-AA₂₋₁₂—P¹)ₙ′)ₙ or V-(M-S—(C═S)—S—K(-AA₂₋₁₂-P¹)ₙ′)ₙ, wherein V, P¹, K, M, n1, n and n′ are as defined above and AA₂₋₁₂ is a linker (c1) as defined above, and preferably wherein at least one—e.g. two, three, four, or more than four—of V, P¹, K, M, n and n′ is/are defined as follows:
(α) V is a peptide of formula (10e), (10f) or (14f), most preferably (10f) or (14f);
(β) P¹ is a moiety derived from NOTA, DOTA, NODAGA, DTPA, N3, TZ, TCO, DBCO, BCN, auristatin (e.g. MMAE), DM4 and PNU-159582;
(δ) K is a branching group of formula (3d) or (3e), wherein preferably m1 is 1 and/or m2 is 1;
(ε) M is a group of formula (5a) or (5l), preferably (5a);
(ζ) n is 1; and
(η) n′ is 2.

If (β) P¹ is a moiety derived from auristatin, e.g. MMAE, (γ) AA₂₋₁₂ represents preferably a cleavable linker comprising a Val-Cit unit, a Val-Ala unit or a Val-Cit-PABC unit, more preferably a Val-Cit-PABC unit. If (β) P¹ is a moiety derived from PNU-159582, (γ) AA₂₋₁₂ represents preferably a cleavable linker comprising a Val-Cit-PABC-DMEA unit.

According to a preferred embodiment, in the above formulae, all of V, K, M, n and n′ are defined as follows while P¹ is preferably as defined above under item (β):
(α) V is a peptide of formula (10e), (10f) or (14f), most preferably (10f) or (14f);
(δ) K is a branching group of formula (3d) or (3e), wherein m1 is 1 and/or m2 is 1;
(ε) M is a group of formula (5a) or (5l), preferably (5a);
(ζ) n is 1; and
(η) n′ is 2.

According to one embodiment, the compound of the present invention is a compound represented by the formula V—O—(C═O)—O—(CH₂)₂₋₆—P¹, V—O—(C═O)—(CH₂)₂₋₆—P¹, V—S—(C═O)—(CH₂)₂₋₆—P¹, V—(S—(C═O)—O—(CH₂)₂₋₆—P¹, V—O—(C═S)—O—(CH₂)₂₋₆—P¹, V—O—(C═O)—S—(CH₂)₂₋₆—P¹, V—(S—(C═O)—S—(CH₂)₂₋₆—P¹, V—(S—(C═S)—O—(CH₂)₂₋₆—P¹, V—O—(C═S)—S—(CH₂)₂₋₆—P¹, V—(S—(C═S)—(CH₂)₂₋₆—P¹, V—O—(C═O)—NH—(CH₂)₂₋₆—P¹, V—S—(C═S)—S—(CH₂)₂₋₆—P¹, V—O—(C═O)—O—K(—(CH₂)₂₋₆—P¹)₂, V—O—(C═O)—K(—(CH₂)₂₋₆—P¹)₂, V—(S—(C═O)—K(—(CH₂)₂₋₆—P¹)₂, V—(S—(C═O)—O—K(—(CH₂)₂₋₆—P¹)₂, V—O—(C═S)—O—K(—(CH₂)₂₋₆—P¹)₂, V—O—(C═O)—S—K(—(CH₂)₂₋₆—P¹)₂, V—(S—(C═O)—S—K(—(CH₂)₂₋₆—P¹)₂, V—(S—(C═S)—O—K(—(CH₂)₂₋₆—P¹)₂, V—O—(C═S)—S—K(—(CH₂)₂₋₆—P¹)₂, V—(S—(C═S)—K(—(CH₂)₂₋₆—P¹)₂, V—O—(C═O)—NH—K(—(CH₂)₂₋₆—P¹)₂, V—(S—(C═S)—S—K(—(CH₂)₂₋₆—P¹)₂, V-M-O—(C═O)—O—(CH₂)₂₋₆—P¹, V-M-O—(C═O)—(CH₂)₂₋₆—P¹, V-M-S—(C═O)—(CH₂)₂₋₆—P¹, V-M-S—(C═O)—O—(CH₂)₂₋₆—P¹, V-M-O—(C═S)—O—(CH₂)₂₋₆—P¹, V-M-O—(C═O)—S—(CH₂)₂₋₆—P¹, V-M-S—(C═O)—S—(CH₂)₂₋₆—P¹, V-M-S—(C═S)—O—(CH₂)₂₋₆—P¹, V-M-O—(C═S)—S—(CH₂)₂₋₆—P¹, V-M-S—(C═S)—(CH₂)₂₋₆—P¹, V-M-O—(C═O)—NH—(CH₂)₂₋₆—P¹, V-M-S—(C═S)—S—(CH₂)₂₋₆—P¹, V-M-O—(C═)—O—K(—(CH₂)₂₋₆—P¹)₂, V-M-O—(C═O)—K(—(CH₂)₂₋₆—P¹)₂, V-M-S—(C═O)—K(—(CH₂)₂₋₆—P¹)₂, V-M-S—H(—)OK(CH₂)₂₋₆—P¹)₂, V-M-O—(C═S)—O—K(—(CH₂)₂₋₆—P¹)₂, V-M-O—(C═O)—S—K(—(CH₂)₂₋₆—P¹)₂, V-M-S—(C═O)—S—K(—(CH₂)₂₋₆—P¹)₂, V-M-S—(C═S)—O—K(—CH₂)₂₋₆—P¹)₂, V-M-O—(C═S)—S—K(—(CH₂)₂₋₆—P¹)₂, V-M-S—(C═S)—K(—(CH₂)₂₋₆—P¹)₂, V-M-O—(C═O)—NH—K(—(CH₂)₂₋₆—P¹)₂, V-M-S—(C═S)—S—K(—(CH₂)₂₋₆—P¹)₂, V—O—(O)—O—NH—CH₂CH₂O)ₙ₁—CH₂CH₂—P¹, V—O—(C═O)—(CH₂CH₂O)ₙ₁—CH₂CH₂—P¹, V—(S—(C═O)—NH—(CH₂CH₂O)ₙ₁—CH₂CH₂—P¹, V—(S—(C═O)—O—NH—(CH₂CH₂O)ₙ₁—CH₂CH₂—P¹, V—O—(C═S)—O—NH—(CH₂CH₂O)ₙ₁—CH₂CH₂—P¹, V—O—(C═O)—S—NH—(CH₂CH₂O)ₙ₁—CH₂CH₂—P¹, V—S—(C═O)—S—NH—(CH₂CH₂O)ₙ₁—CH₂CH₂—P¹, V—(S—(C═S)—O—NH—(CH₂CH₂O)ₙ₁—CH₂CH₂—P¹, V—O—(C═S)—S—NH—(CH₂CH₂O)ₙ₁—CH₂CH₂—P¹, V—(S—(C═S)—NH—(CH₂CH₂O)ₙ₁—CH₂CH₂—P¹, V—O—(C═O)—NH—NH—(CH₂CH₂O)ₙ₁—CH₂CH₂—P¹, V—S—(C═S)—S—NH—(CH₂CH₂O)ₙ₁—CH₂CH₂—P¹, V—O—(C═O)—O—K(—NH—(CH₂CH₂O)ₙ₁—CH₂CH₂—P¹)₂, V—O—(C═O)—K(—NH—(CH₂CH₂O)ₙ₁—CH₂CH₂—P¹)₂, V—(S—(C═O)—K(—NH—(CH₂CH₂O)ₙ₁—CH₂CH₂—P¹)₂, V—(S—(C═O)—O—K(—NH—(CH₂CH₂O)ₙ₁—CH₂CH₂—P¹)₂, V—O—(C═S)—O—K(—NH—(CH₂CH₂O)ₙ₁—CH₂CH₂—P¹)₂, V—O—(C═O)—S—K(—NH—(CH₂CH₂O)ₙ₁—CH₂CH₂—P¹)₂, V—(S—(C═O)—S—K(—NH—(CH₂CH₂O)ₙ₁—CH₂CH₂—P¹)₂, V—(S—(C═S)—O—K(—NH—(CH₂CH₂O)ₙ₁—CH₂CH₂—P¹)₂, V—O—(C═S)—S—K(—NH—(CH₂CH₂O)ₙ₁—CH₂CH₂—P¹)₂, V—(S—(C═S)—K(—NH—(CH₂CH₂O)ₙ₁—CH₂CH₂—P¹)₂, V—O—(C═O)—NH—K(—NH—(CH₂CH₂O)ₙ₁—CH₂CH₂—P¹)₂, V—(S—(C═S)—S—K(—NH—(CH₂CH₂O)ₙ₁—CH₂CH₂—P¹)₂, V-M-O—(C═O)—O—NH—(CH₂CH₂O)ₙ₁—CH₂CH₂—P¹, V-M-O—(C═O)—NH—(CH₂CH₂O)ₙ₁—CH₂CH₂—P¹, V-M-S—(C═O)—NH—(CH₂CH₂O)ₙ₁—CH₂CH₂—P¹, V-M-S—(C═O)—O—NH—(CH₂CH₂O)ₙ₁—CH₂CH₂—P¹, V-M-O—(C═S)—O—NH—(CH₂CH₂O)ₙ₁—CH₂CH₂—P¹, V-M-O—(C═O)—S—NH—(CH₂CH₂O)ₙ₁—CH₂CH₂—P¹, V-M-S—(C═O)—S—NH—(CH₂CH₂O)ₙ₁—CH₂CH₂—P¹, V-M-S—(C═S)—O—NH—(CH₂CH₂O)ₙ₁—CH₂CH₂—P¹, V-M-O—(C═S)—S—NH—(CH₂CH₂O)ₙ₁—CH₂CH₂—P¹, V-M-S—(C═S)—NH—(CH₂CH₂O)ₙ₁—CH₂CH₂—P¹, V-M-O—(C═O)—NH—NH—(CH₂CH₂O)ₙ₁—CH₂CH₂—P¹, V-M-S—(C═S)—S—NH—(CH₂CH₂O)ₙ₁—CH₂CH₂—P¹, V-M-O—(C═O)—O—K(—NH—(CH₂CH₂O)ₙ₁—CH₂CH₂—P¹)₂, V-M-O—(C═O)—K(—NH—(CH₂CH₂O)ₙ₁—CH₂CH₂—P¹)₂, V-M-S—(C═O)—K(—NH—(CH₂CH₂O)ₙ₁—CH₂CH₂—P¹)₂, V-M-S—(C═O)—O—K(—NH—(CH₂CH₂O)ₙ₁—CH₂CH₂—P¹)₂, V-M-O—(C═S)—O—K(—NH—(CH₂CH₂O)ₙ₁—CH₂CH₂—P¹)₂, V-M-O—(C═O)—S—K(—NH—(CH₂CH₂O)ₙ₁—CH₂CH₂—P¹)₂, V-M-S—(C═O)—S—K(—NH—(CH₂CH₂O)ₙ₁—CH₂CH₂—P¹)₂, V-M-S—(C═S)—O—K(—NH—(CH₂CH₂O)ₙ₁—CH₂CH₂—P¹)₂, V-M-O—(C═S)—S—K(—NH—(CH₂CH₂O)ₙ₁—CH₂CH₂—P¹)₂, V-M-S—(C═S)—K(—NH—(CH₂CH₂O)ₙ₁—CH₂CH₂—P¹)₂, V-M-O—(C═O)—NH—K(—NH—(CH₂CH₂O)ₙ₁—CH₂CH₂—P¹)₂, V-M-S—(C═S)—S—K(—NH—(CH₂CH₂O)ₙ₁—CH₂CH₂—P¹)₂, V—O—(C═O)—O-AA₂₋₁₂-P¹, V—O—(C═O)-AA₂₋₁₂-P¹, V—(S—(C═O)-AA₂₋₁₂-P¹, V—(S—(C═O)—O-AA₂₋₁₂-P¹, V—O—(C═S)—O-AA₂₋₁₂-P¹, V—O—(C═O)—S-AA₂₋₁₂-P¹, V—(S—(C═O)—S-AA₂₋₁₂-P¹, V—(S—(C═S)—O-AA₂₋₁₂—P¹, V—O—(C═S)—S-AA₂₋₁₂-P¹, V—(S—(C═S)-AA₂₋₁₂-P¹, V—O—(C═O)—NH-AA₂-12—P¹, V—(S—(C═S)—S-AA₂₋₁₂-P¹, V—O—(C═O)—O—K(-AA₂₋₁₂-P¹)₂, V—O—(C═O)—K(-AA₂₋₁₂-P¹)₂, V—(S—(C═O)—K(-AA₂₋₁₂-P¹)₂, V—(S—(C═O)—O—K(-AA₂₋₁₂-P¹)₂, V—O—(C═S)—O—K(-AA₂₋₁₂—P¹)₂, V—O—(C═O)—S—K(-AA₂₋₁₂-P¹)₂, VS—O)—(—S—K(-AA₂₋₁₂-P¹)₂, V—S—(C═S)—O—K(-AA₂₋₁₂-P¹)₂, V—O—(C═S)—S—K(-AA₂₋₁₂-P¹)₂, V—(S—(C═S)—K(-AA₂₋₁₂-P¹)₂, V—O—(C═O)—NH—K(-AA₂₋₁₂-P¹)₂, V—(S—(C═S)—S—K(-AA₂₋₁₂-P¹)₂, V-M-O—(C═O)—O-AA₂₋₁₂—P¹, V-M-O—(C═O)-AA₂₋₁₂-P¹, V-M-S—(C═O)-AA₂₋₁₂-P¹, V-M-S—(C═O)—O-AA₂₋₁₂—P¹, V-M-O—(C═S)—O-AA₂₋₁₂-P¹, V-M-O—(C═O)—S-AA₂₋₁₂-P¹, V-M-S—(C═O)—S-AA₂₋₁₂—P¹, V-M-S—(C═S)—O-AA₂₋₁₂-P¹, V-M-O—(C═S)—S-AA₂₋₁₂-P¹, V-M-S—(C═S)-AA₂₋₁₂-P¹, V-M-O—(C═O)—NH-AA₂₋₁₂-P¹, V-M-S—(C═S)—S-AA₂₋₁₂-P¹, V-M-O—(C═O)—O—K(-AA₂₋₁₂-P¹)₂, V-M-O—(C═O)—K(-AA₂₋₁₂-P¹)₂, V-M-S—(C═O)—K(-AA₂-12—P¹)₂, V-M-S—(C═O)—O—K(-AA₂₋₁₂-P¹)₂, V-M-O—(C═S)—O—K(-AA₂₋₁₂-P¹)₂, V-M-O—(C═O)—S—K(-AA₂₋₁₂-P¹)₂, V-M-S—(C═O)—S—K(-AA₂₋₁₂-P¹)₂, V-M-S—(C═S)—O—K(-AA₂-12—P¹)₂, V-M-O—(C═S)—S—K(-AA₂₋₁₂-P¹)₂, V-M-S—(C═S)—K(-AA₂₋₁₂-P¹)₂, V-M-O—(C═O)—NH—K(-AA₂₋₁₂-P¹)₂, V-M-S—(C═S)—S—K(-AA₂₋₁₂-P¹)₂, wherein V, P¹, K, M and n1 are as defined above and AA₂₋₁₂ is a linker (c1) as defined above, and preferably wherein at least one—e.g. two, three, four, or more than four—of V, P¹, K and M is/are defined as follows:
(α) V is a peptide of formula (10e), (10f) or (14f), most preferably (10f) or (14f);
(β) P¹ is a moiety derived from NOTA, DOTA, NODAGA, DTPA, N3, TZ, TCO, DBCO, BCN, auristatin (e.g. MMAE), DM4 and PNU-159582;
(δ) K is a branching group of formula (3d) or (3e), wherein preferably m1 is 1 and/or m2 is 1; and
(ε) M is a group of formula (5a) or (5l), preferably (5a).

If (β) P¹ is a moiety derived from auristatin, e.g. MMAE, (γ) AA₂₋₁₂ represents preferably a cleavable linker comprising a Val-Cit unit, a Val-Ala unit or a Val-Cit-PABC unit, more preferably a Val-Cit-PABC unit. If (β) P¹ is a moiety derived from PNU-159582, (γ) AA₂₋₁₂ represents preferably a cleavable linker comprising a Val-Cit-PABC-DMEA unit.

According to a preferred embodiment, in the above formulae, all of V, K and M are defined as follows while P¹ is preferably as defined above under item (β):
(α) V is a peptide of formula (10e), (10f) or (14f), most preferably (10f) or (14f);
(δ) K is a branching group of formula (3d) or (3e), wherein m1 is 1 and/or m2 is 1; and
(ε) M is a group of formula (5a) or (5l), preferably (5a).

According to a preferred embodiment, the compound of the present invention is a compound represented by the formula V-(M-O—(C═O)—O—P¹)ₙ, V-(M-O—(C═O)—P¹)ₙ, V-(M-S—(C═O)—P¹)ₙ, V-(M-S—(C═O)—O—P¹)ₙ, V-(M-O—(C═S)—O—P¹)ₙ, V-(M-O—(C═O)—S—P¹)ₙ, V-(M-S—(C═O)—S—P¹)ₙ, V-(M-S—(C═S)—O—P¹)ₙ, V-(M-O—(C═S)—S—P¹)ₙ, V-(M-S—(C═S)—P¹)ₙ, V-(M-O—(C═O)—NH—P¹)ₙ, V-(M-S—(C═S)—S—P¹)ₙ, V-(M-O—(C═O)—O-L-P¹)ₙ, V-(M-O—(C═O)-L-P¹)ₙ, V-(M-S—(C═O)-L-P¹)ₙ, V-(M-S—(C═O)—O-L-P¹)ₙ, V-(M-O—(C═S)—O-L-P¹)ₙ, V-(M-O—(C═O)—S-L-P¹)ₙ, V-(M-S—(C═O)—S-L-P¹)ₙ, V-(M-S—(C═S)—O-L-P¹)ₙ, V-(M-O—(C═S)—S-L-P¹)ₙ, V-(M-S—(C═S)-L-P¹)ₙ, V-(M-O—(C═O)—NH-L-P¹)ₙ, V-(M-S—(C═S)—S-L-P¹)ₙ, V-(M-O—(C═O)—O—K(-L-P¹)ₙ′)ₙ, V-(M-O—(C═O)—K(-L-P¹)ₙ′)ₙ, V-(M-S—(C═O)—K(-L-P¹)ₙ′)ₙ, V-(M-S—(C═O)—O—K(-L-P¹)ₙ′)ₙ, V-(M-O—(C═S)—O—K(-L-P¹)ₙ′)ₙ, V-(M-O—(C═O)—S—K(-L-P¹)ₙ′)ₙ, V-(M-S—(C═O)—S—K(-L-P¹)ₙ′)ₙ, V-(M-S—(C═S)—O—K(-L-P¹)ₙ′)ₙ, V-(M-O—(C═S)—S—K(-L-P¹)ₙ′)ₙ, V-(M-S—(C═S)—K(-L-P¹)ₙ′)ₙ, V-(M-O—(C═O)—NH—K(-L-P¹)ₙ′)ₙ, V-(M-S—(C═S)—S—K(-L-P¹)ₙ′)ₙ, V-(M-O—(C═O)—O—K(—P¹)ₙ′)ₙ, V-(M-O—(C═O)—K(—P¹)ₙ′)ₙ, V-(M-S—(C═O)—K(—P¹)ₙ′)ₙ, V-(M-S—(C═O)—O—K(—P¹)ₙ′)ₙ, V-(M- O—(C═S)—O—K(—P¹)ₙ′)ₙ, V-(M-O—(C═O)—S—K(—P¹)ₙ′)ₙ, V-(M-S—(C═O)—S—K(—P¹)ₙ′)ₙ, V-(M-S—(C═S)—O—K(—P¹)ₙ′)ₙ, V-(M-O—(C═S)—S—K(—P¹)ₙ′)ₙ, V-(M-S—(C═S)—K(—P¹)ₙ′)ₙ, V-(M-O—(C═O)—NH—K(—P¹)ₙ′)ₙ or V-(M-S—(C═S)—S—K(—P¹)ₙ′)ₙ, wherein V, P¹, L, K, M, n and n′ are as defined above, and wherein preferably at least one—e.g. two, three, four, or more than four—of V, P¹, L, K, M, n and n′ is/are defined as follows:
(α) V is a peptide of formula (10e), (10f) or (14f), most preferably (10f) or (14f);
(β) P¹ is a moiety derived from NOTA, DOTA, NODAGA, DTPA, N3, TZ, TCO, DBCO, BCN, auristatin (e.g. MMAE), DM4 and PNU-159582;
(γ) L is a linker selected from
   (a1) an alkylene group having 2 to 6 carbons (—(CH₂)₂₋₆-),
   (b1) a polyalkylene group of formula —NH—(CH₂CH₂O)ₙ₁—CH₂CH₂—, n1 being an integer of 0-35, and
   (c1) a peptidic linker comprising 2 to 12 amino acids, which is optionally cleavable, preferably a cleavable peptidic linker comprising a Val-Cit unit, a Val-Ala unit, a Val-Cit-PABC unit or a Val-Cit-PABC-DMEA unit;
(δ) K is a branching group of formula (3d) or (3e), wherein preferably m1 is 1 and/or m2 is 1;
(ε) M is a group of formula (5a) or (5l), preferably (5a);
(ζ) n is 1; and
(η) n′ is 2.

If (β) P¹ is a moiety derived from auristatin, e.g. MMAE, (γ) L represents preferably a cleavable linker comprising a Val-Cit unit, a Val-Ala unit or a Val-Cit-PABC unit, more preferably a Val-Cit-PABC unit. If (β) P¹ is a moiety derived from PNU-159582, (γ) L represents preferably a cleavable linker comprising a Val-Cit-PABC-DMEA unit.

More preferably, in the above formulae, all of V, K, M, n and n′ are defined as follows while one or both of P¹ and L are preferably as defined above under items (β) and (γ):
(α) V is a peptide of formula (10e), (10f) or (14f), most preferably (10f) or (14f);
(δ) K is a branching group of formula (3d) or (3e), wherein m1 is 1 and/or m2 is 1;
(ε) M is a group of formula (5a) or (5l), preferably (5a);
(ζ) n is 1; and
(η) n′ is 2.

In one embodiment, the compound of formula (1) is selected from:

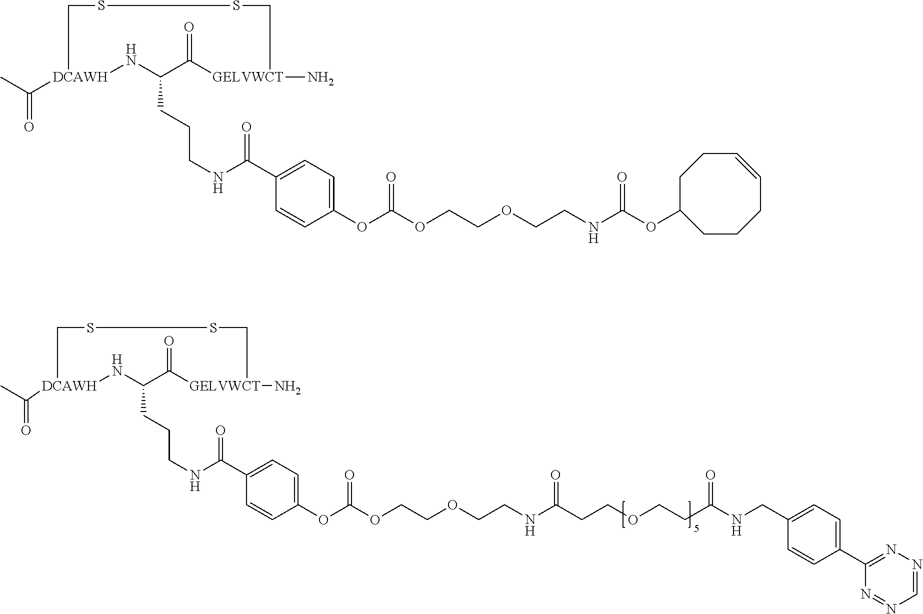

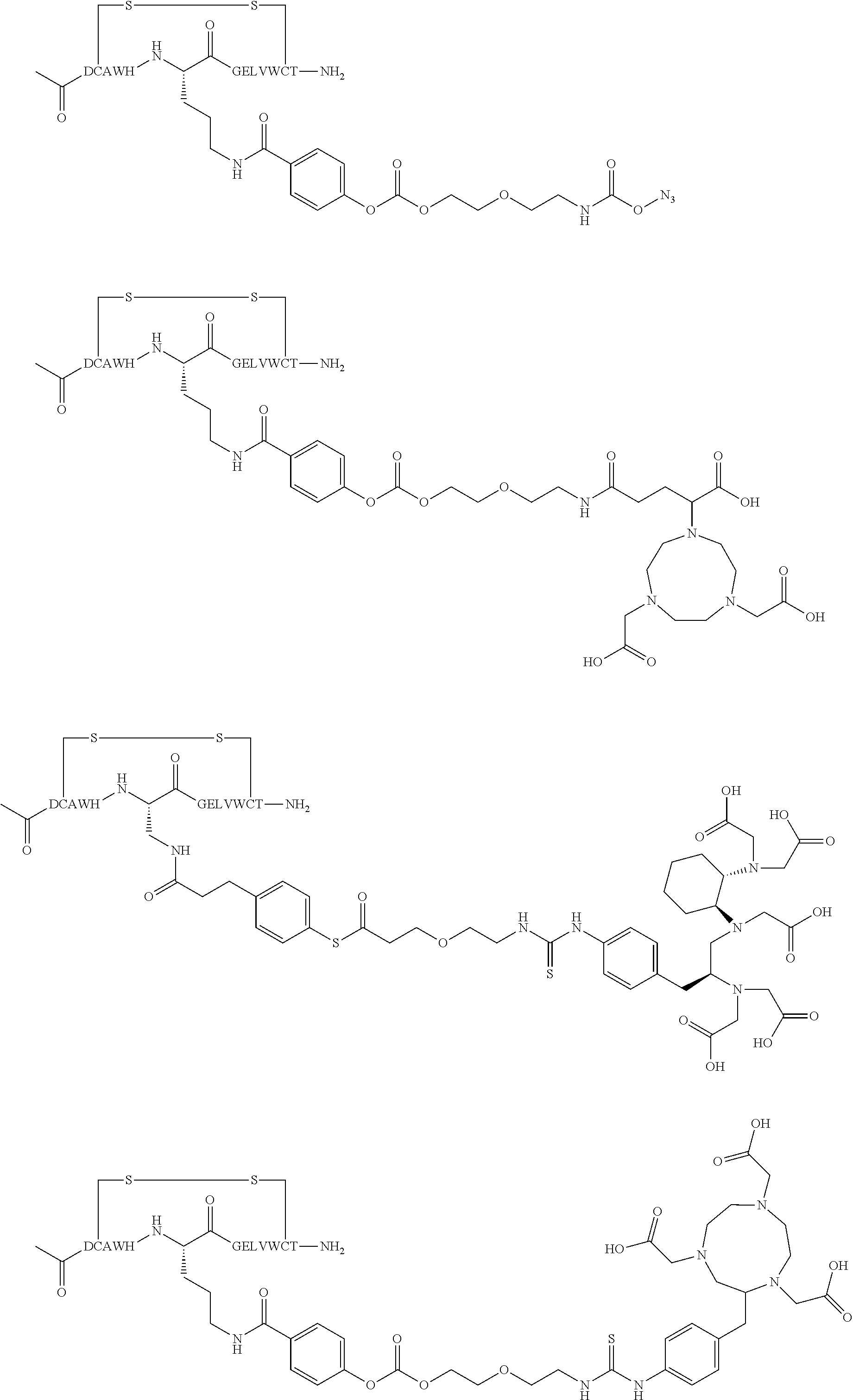

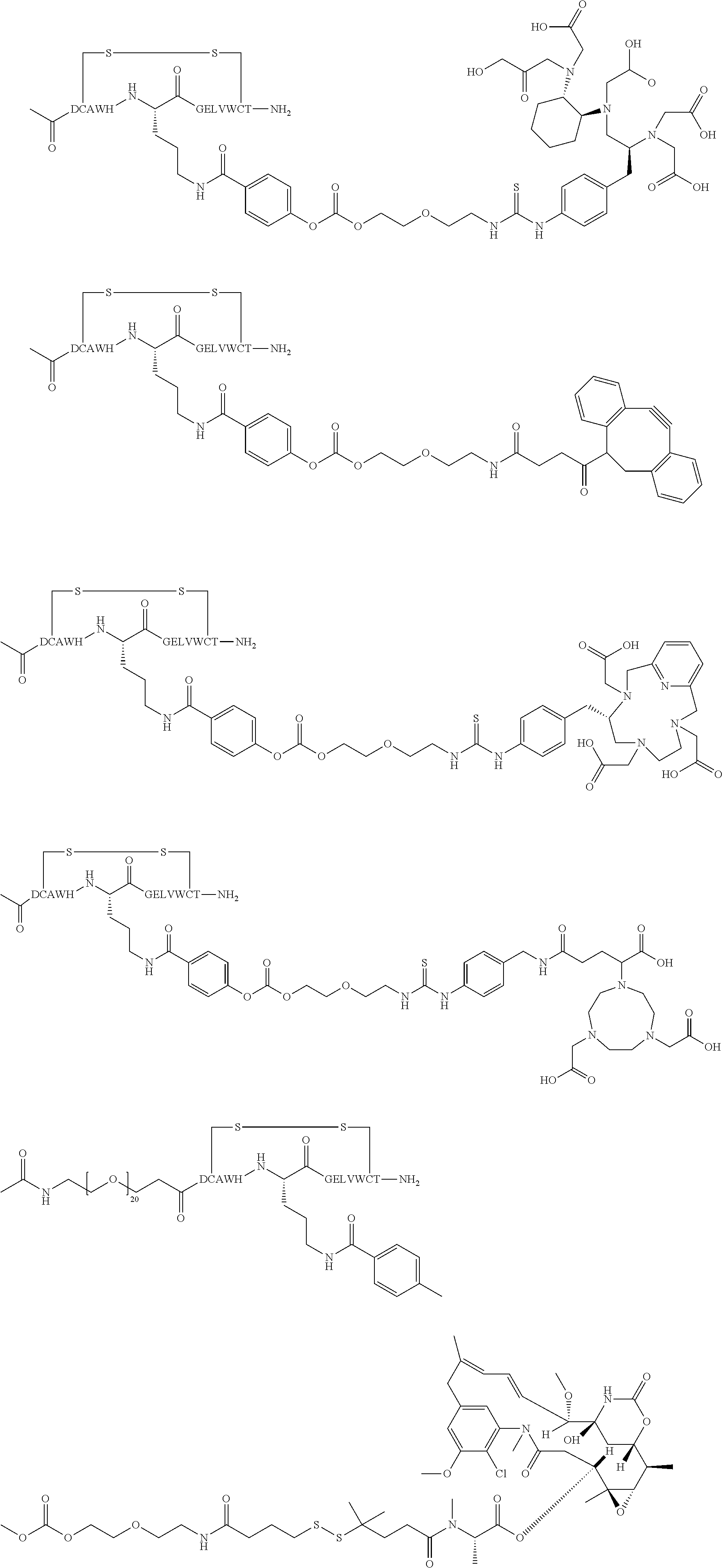

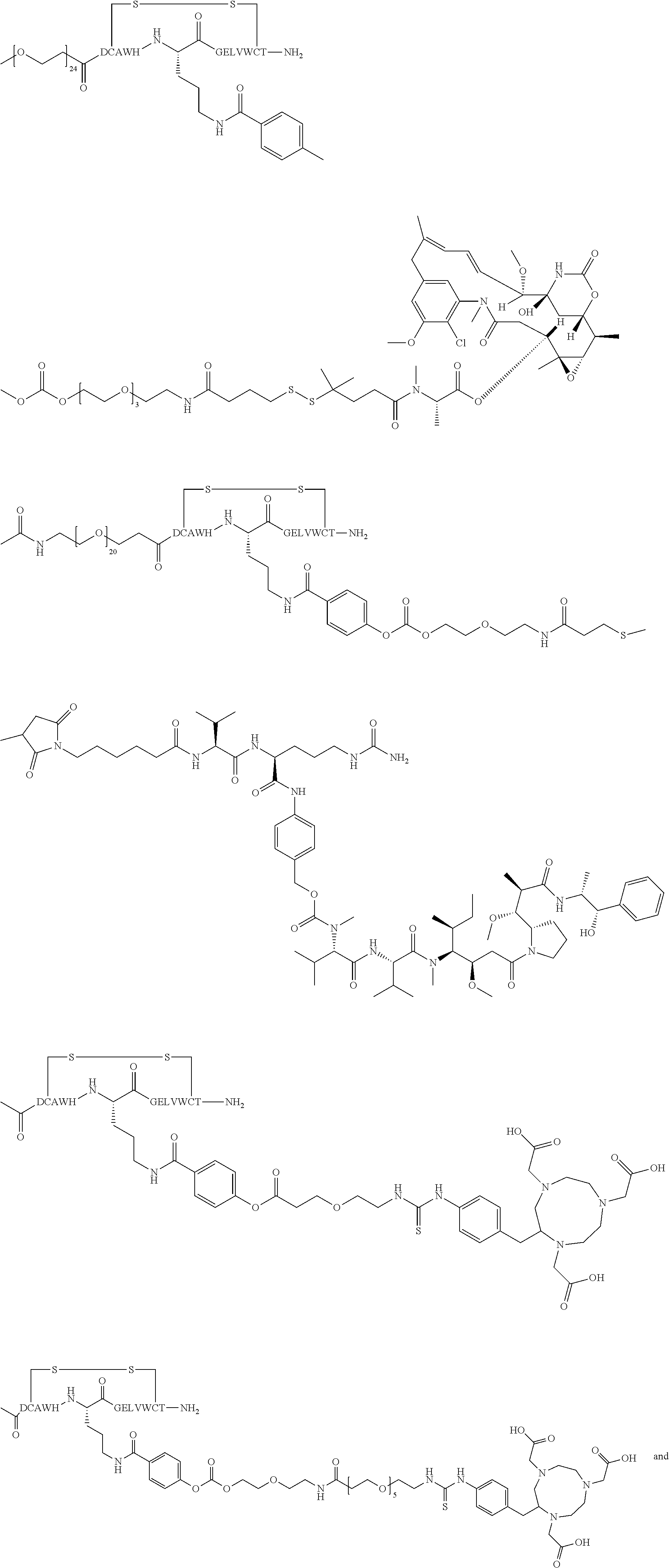

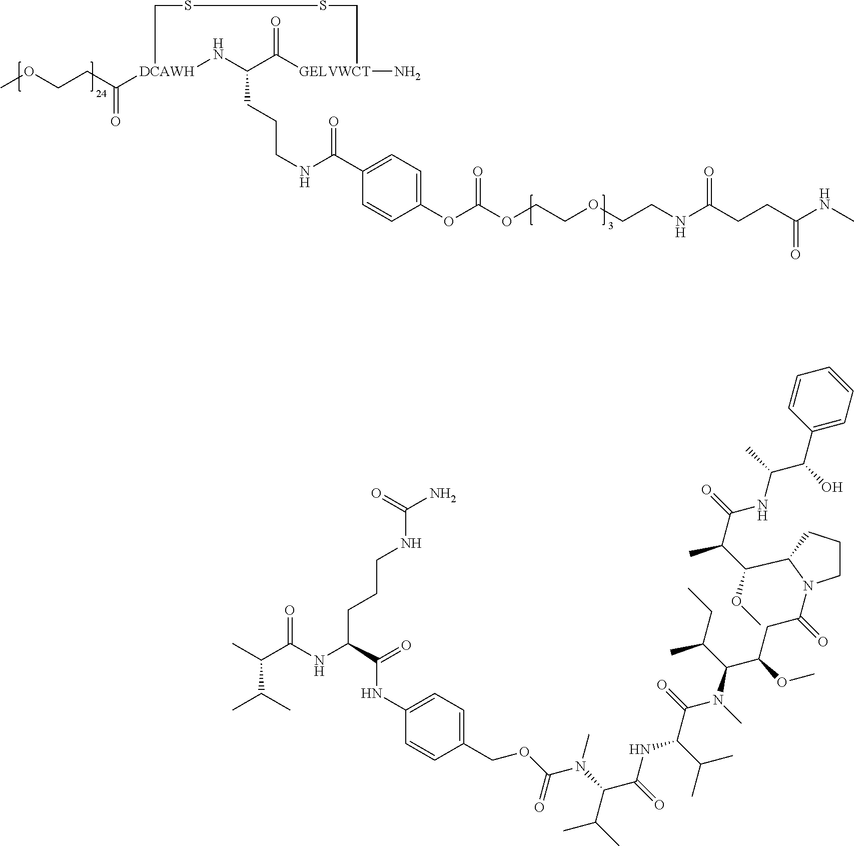

### 4. Kit for the Site-Specific Modification of Antibodies or Antibody Fragments

In some aspects, the present invention relates to a kit comprising the compound described hereinbefore and a buffer, which can be used for the regioselective modification (e.g. for the labelling) of antibodies or fragments thereof, the antibody fragments being optionally incorporated into Fc-fusion proteins, in particular for the regioselective modification of therapeutic antibodies.

The compound of the present invention and the buffer (together forming the kit) can be presented individually, e.g. in separate primary containers (which may be shipped to the customer in a single box), which can be stored for a prolonged period, without degradation. The compound and buffer can be formulated and proportioned for a given amount of antibody or fragment thereof to be modified. In some aspects, the compound of the present invention is presented as a solid (e.g. as a lyophilized powder, or non-covalently adsorbed or covalently bound to a solid phase matrix as described further below), or as a solution in a suitable solvent, such as a water-miscible, polar aprotic solvent (e.g. DMF, DMSO), which can be mixed with the buffer shortly prior to antibody or antibody fragment modification.

The buffer to be used in the kit of the present invention is not particularly limited. Preferably, the buffer has a pH of 5.5 to 11, more preferably of 7.0 to 9.5. The buffer can be selected from e.g. 2-bis(2-hydroxyethyl)amino acetic acid (Bicine), carbonate-bicarbonate, tris(hydroxymethyl)methylamino propane sulfonic acid (TAPS), 4-(2-hydroxyethyl)-1-piperazineethane sulfonic acid (HEPES). Preferably, the buffer is a carbonate-bicarbonate or bicine buffer with a pH of 7.0 to 9.5, e.g. a pH of about 9.0.

According to one embodiment, the compound of the present invention is immobilized on a solid phase matrix (solid support), e.g. immobilized on beads. The compound can be immobilized using methods known in the art such as high-affinity (e.g. biotin-streptavidin, biotin-neutravidin) binding, “click” chemistry (as defined by Kolb et al. in “Click Chemistry: Diverse Chemical Function from a Few Good Reactions” Angewandte Chemie Int. Ed. 2001, 40(11), 2004-2021), hydrazone ligation etc. Preferably, the solid phase matrix is an inert matrix, such as a polymeric gel, comprising a three-dimensional structure, lattice or network of material. More preferably, the solid phase matrix is a material used for affinity chromatography such as a xerogel. Such gels shrink on drying to a compact solid comprising only the gel matrix. When the dried xerogel is resuspended in a liquid, the gel matrix imbibes the liquid, swells and returns to the gel state. Examples of xerogels which can be suitably used in the present invention include polymeric gels, such as cellulose, crosslinked-dextran gels (e.g. Sephadex®), agarose, cross-linked agarose, polyacrylamide gels, polyacrylamide-agarose gels.

Preferably, the compound is immobilized on the solid support via a biotin-streptavidin interaction, via a covalent linkage obtained by click reaction between an alkyne and azide, via covalent linkage obtained by reaction between a thiol and an acetamide, via covalent linkage obtained by reaction between a derivative of TCO and a derivative of TZ, or via covalent linkage obtained by reaction between a thiol and maleimide.

In one embodiment, the compound is immobilized on the solid support by means of the conjugation group Y¹ or Y² in formula (7a), e.g. by high-affinity binding such as biotin-streptavidin or biotin-neutravidin binding (in this case, Y¹/Y² represents e.g. a biotin-containing group), by click chemistry (in this case, Y¹/Y² represents e.g. a DBCO—, azide- or alkyne-containing group).

### 5. Use of Reactive Conjugates in Methods for the Regioselective Modification of Antibodies or Antibody Fragments

The compound of the present invention can be used in a method for the regioselective modification of an antibody or fragment thereof, the antibody fragment being optionally incorporated into an Fc-fusion protein. The method produces a modified antibody or modified antibody fragment, e.g. an ADC, which can be used in a method of diagnosing, monitoring e.g. monitoring the effectiveness of a treatment e.g. over time, imaging or treating disease as described further below. The antibody or fragment thereof to be modified can be selected by a physician depending on the disease to be treated based on established medical guidelines.

In one embodiment, the method comprises the step of reacting (contacting) an antibody or fragment thereof with the compound, which may be comprised in the kit described hereinbefore. The reaction mixture can be purified by techniques known in the art such as gel permeation chromatography using a suitable solvent. Examples of suitable stationary phases for isolating the clean conjugate include polyacrylamide gels such as Bio-Gel® P-30 and crosslinked dextrans such as Sorbadex®, Zetadex® or Sephadex®.

When the compound of the present invention is immobilized on a solid support, the immobilized compound is contacted with a sample containing the antibody or antibody fragment to be modified, and thereafter the solid support is washed with a suitable solvent that will remove substantially all the material in the sample except the antibody, which is bound to the solid support. Finally, the solid support is washed with another suitable solvent, such as a glycine buffer at pH 2.5 that will release the modified antibody/antibody fragment, e.g. the ADC, from the solid support.

The method of the present invention can be applied to any antibody (e.g. IgG protein), antibody fragment, or Fc-fusion protein provided that it comprises an Fc region for interaction with the vector. In one embodiment, the antibody to be modified is a monoclonal antibody (mAb), preferably an antibody selected from the group consisting of adalimumab, aducanumab, alemtuzumab, altumomab pentetate, atezolizumab, anetumab, avelumab, bapineuzumab, basiliximab, bectumomab, bermekimab, besilesomab, bevacizumab, bezlotoxumab, brentuximab, brentuximab vedotin, brodalumab, catumaxomab, cemiplimab, cetuximab, cinpanemab, clivatuzumab, crenezumab, tetraxetan, daclizumab, daratumumab, denosumab, dinutuximab, durvalumab, edrecolomab, elotuzumab, emapalumab, enfortumab, enfortumab vedotin, epratuzumab, epratuzumab-SN-38, etaracizumab, gemtuzumab, gemtuzumab ozogamycin, girentuximab, gosuranemab, ibritumomab, inebilizumab infliximab, inotuzumab, inotuzumab ozogamicin, ipilimumab, isatuximab ixekizumab, J591 PSMA-antibody, labetuzumab, lecanemab, mogamulizumab, necitumumab, nimotuzumab, natalizumab, nivolumab, ocrelizumab, ofatumumab, olaratumab, oregovomab, panitumumab, pembrolizumab, pertuzumab, polatuzumab, polatuzumab vedotin, prasinezumab, racotumomab, ramucirumab, rituximab, sacituzumab, sacituzumab govitecan, semorinemab, siltuximab, solanezumab, tacatuzumab, teprotumumab, tilavonemab, tocilizumab, tositumomab, trastuzumab, trastuzumab deruxtecan, trastuzumab emtansine, TS23, ustekinumab, vedolizumab, votumumab, zagotenemab, zalutumumab, zanolimumab, fragments and derivatives thereof; more preferably atezolizumab, durvalumab, pembrolizumab, rituximab or trastuzumab.

In one embodiment, the antibody or fragment thereof to be modified is contained in a commercially formulated antibody, preferably contained in a commercially formulated antibody having a marketing authorization delivered by the EMA or the Food and Drug Administration (FDA) of the United States of America. According to one embodiment, the commercially formulated antibody is selected from Humira®, Lemtrada®, Campath®, Tecentriq®, Bavencio®, Simulect®, LymphoScan®, Xilonix®, Scintimun®, Avastin®, Zinplava®, Blincyto®, Libtayo®, Erbitux®, hPAM4-Cide®, Zenapax®, Darzalex®, Prolia®, Unituxin®, Imfinzi®, Panorex®, Empliciti®, Gamifant®, Rencarex®, Remicade®, Besponsa®, Yervoy®, CEA-Cide®, Poteligeo®, Tysabri®, Portrazza®, Theracim®, Opdivo®, Arzerra®, Lartruvo®, Omnitarg®, Vaxira®, Cyramza®, MabThera®, Rituxan®, Sylvant®, Bexxar®, Herceptin®, Kadcyla®, Stelara®, HuMax-EGFr®, HuMax-CD4®, and biosimilars thereof; preferably from MabThera® and Herceptin®.

Commercially available antibodies are often formulated with Histidine for stability. When a commercially available antibody is mixed with a reactive conjugate, histidine would be expected to react in competitive manner with the reactive center and therefore degrade the reactive conjugate, which would lead to decreased yields with respect to the ADCs. However, the inventors surprisingly found that with the compounds of the invention the yield is not impacted, or significantly impacted. Without wishing to be bound by theory the inventors believe that this is due to an increased rate of reaction between the compounds of the invention and the amino acids on the side chain of the antibody or antibody fragment e.g. lysine or cysteine. This favorable kinetic is likely linked to the dramatic increase of local concentration of the reactive centers in the vicinity of the targeted amino acid upon binding of the vector to the Fc fragment.

In one embodiment, the antibody fragment to be modified is incorporated into an Fc-fusion protein, which is preferably selected from belatacept, aflibercept, ziv-aflibercept, dulaglutide, rilonacept, romiplostim, abatacept and alefacept.

### 6. Modified Antibodies or Modified Antibody Fragments

The modified antibodies and modified antibody fragments obtained by (or obtainable by) reacting the compound of the present invention with antibodies or antibody fragments (the antibody fragments being optionally incorporated into Fc-fusion proteins) comprise one or more payloads attached thereto via a divalent group, which is a group derived from the reactive moiety (Y) of formula (1) (i.e. it corresponds to the reactive moiety of formula (1) which has been reacted with the side chain of an amino acid exposed at the surface of an antibody or fragment thereof).

According to one embodiment, the modified antibody or modified antibody fragment is represented by the following formula (11):

(P—W)ₚ-A (11)
wherein,
P is a group comprising one or more payloads (P¹) as described above, wherein P is preferably a group of formula (2a);
W is F1-RC′, wherein F1 is attached to P and RC′ is a moiety derived from a reactive center (RC) attached to A, F1 and RC being as defined in formulae (4a) and (4b);
A is moiety derived from an antibody or an antibody fragment optionally incorporated into an Fc-fusion protein, said antibody or antibody fragment being as defined above; and
p is an integer of 1 to 5. Preferably, p is 1 to 3, more preferably 1 or 2.

In those instances where a reactive moiety reacts with the side chain of a lysine residue, the attachment of the group (P) to the antibody or antibody fragment occurs via a nitrogen atom-containing group such as an amide group, an urethane group, a thiourethane group, a dithiourethane group, etc. For example, if the compound of the invention includes a reactive moiety of formula (4a′) or (6a), the divalent group (W) in formula (11) is an urethane group in which the nitrogen atom forms part of the Lys side chain. If the compound includes a reactive moiety of formula (4e′), the divalent group (W) is a thiourethane group. According to one embodiment, the modified antibody or modified antibody fragment is represented by one of the following formulae (12a) to (12c):

(P¹-L-W)ₚ-A (12a)

((P¹-L)ₙ′-K—W)ₚ-A (12b)

((P¹)ₙ′-K—W)ₚ-A (12c)
wherein,
P¹, L, K, W, A, n′ and p are as defined above.

In a preferred embodiment, the modified antibody or modified antibody fragment is represented by the formula (12a) or (12b), more preferably by formula (12a).

In the above formulae, p represents the Degree of Conjugation (DoC) of the modified antibody or modified antibody fragment. The compound of the invention may produce modified antibodies or modified antibody fragments, e.g. an ADC, having high DAR values. In particular, the antibody or antibody fragment may be modified by attachment of a payload-containing group at multiple conjugation sites (p>1), wherein each payload-containing group may comprise multiple payload molecules (n′>1).

### 7. Use of Modified Antibodies or Modified Antibody Fragments for Diagnostic and/or Therapeutic Purposes

The modified antibodies and modified antibody fragments obtained by (or obtainable by) reacting the compound of the present invention with antibodies or antibody fragments (the antibody fragments being optionally incorporated into Fc-fusion proteins) can be used to diagnose, monitor, image or treat a disease, in particular cancer, and/or monitor or image a treatment of said disease. The treatment can be a therapeutic and/or prophylactic treatment, with the aim being to prevent, reduce or stop an undesired physiological change or disorder. In some instances, the treatment can prolong survival of a subject as compared to expected survival if not receiving the treatment.

The disease that is treated by the modified antibody or modified antibody fragment (e.g. the ADC) can be any disease that benefits from the treatment, including chronic and acute disorders or diseases and also those pathological conditions which predispose to the disorder. In some instances, the disease is a neoplastic disease such as cancer that can be treated via the targeted destruction of tumor cells. Non-limiting examples of cancers that may be treated include benign and malignant tumors, either solid or liquid; leukemia and lymphoid malignancies, as well as breast, ovarian, stomach, endometrial, salivary gland, lung, kidney, colon, thyroid, pancreatic, prostate or bladder cancer. The disease may be a neuronal, glial, astrocytal, hypothalamic or other glandular, macrophagal, epithelial, stromal and blastocoelic disease; or inflammatory, angiogenic or an immunologic disease. An exemplary disease is a solid, malignant tumor.

According to one embodiment, the disease or treatment thereof is selected from the group consisting of Alzheimer's Disease, Amyotrophic Lateral Sclerosis, Cerebral Arteriosclerosis, Encephalopathy, Huntington's Disease, Multiple Sclerosis, Parkinson's Disease, Progressive Multifocal Leukoencephalopathy, Systemic Lupus Erythematosus, systemic sclerosis, Angina (including unstable angina), Aortic aneurysm, Atherosclerosis, Cardiac transplant, Cardiotoxicity diagnosis, Coronary artery bypass graft, Heart failure (including atrial fibrillation terminated systolic heart failure), hypercholesterolaemia, Ischemia, Myocardial infarction, Thromboembolism, Thrombosis, Ankylosing spondylitis, Autoimmune cytopenias, Autoimmune myocarditis, Crohn's disease, Graft Versus Host disease, Granulomatosis with Polyangiitis, Idiopathic thrombocytopenic purpura, Juvenile arthritis, Juvenile diabetes (Type 1 diabetes), Lupus, Microscopic polyangiitis, Multiple sclerosis, Plaque psoriasis, Psoriasis, Psoriatic arthritis, Rheumatoid arthritis, Ulcerative colitis (UC), Uveitis, and Vasculitis.

According to one embodiment, the disease to be treated involves cells selected from lymphoma cells, myeloma cells, renal cancer cells, breast cancer cells, prostate cancer cells, ovarian cancer cells, colorectal cancer cells, gastric cancer calls, squamous cancer cells, small-cell lung cancer cells, testicular cancer cells, pancreatic cancer cells, liver cancer cells, melanoma, head-and-neck cancer cells, and any cells growing and dividing at an unregulated and quickened pace to cause cancers; preferably selected from breast cancer cells, small-cell lung cancer cells, lymphoma cells, colorectal cancer cells, and head-and-neck cancer cells.

According to one embodiment, the modified antibody or modified antibody fragment is used in a method of diagnosing, monitoring e.g. monitoring the effectiveness of a treatment e.g. over time, imaging and/or treating a disease (e.g. cancer) by administering the modified antibody or modified antibody fragment to a subject (e.g. a patient).

The molecule can be administered to a subject at one time or over a series of treatments. Depending on the type and severity of the disease, and/or on the payload, and/or on the antibody or antibody fragment, between about 0.1 μg/kg to 1 mg/kg of drug may be used as an initial candidate dosage for first administration in a first-in-human trial, e.g. by one or more separate administrations, or by continuous infusion. A typical daily dosage can range from about 0.1 mg/kg to 50 mg/kg or more, or from about 0.5 to about 30 mg/kg e.g. 0.5 to about 25 mg/kg of patient weight. However, typical dosages will depend on a variety of factors including the specific payload (active agent), the age, body weight, general health, sex and diet of the subject; whether administration is for imaging, monitoring or treatment purposes, and other factors well known in the medical art.

When treating cancer, the therapeutic effect that is observed can be a reduction in the number of cancer cells; a reduction in tumor size; inhibition or retardation of cancer cell infiltration into peripheral organs; inhibition of tumor growth; and/or relief of one or more of the symptoms associated with cancer.

According to a preferred embodiment, the modified antibody or modified antibody fragment is administered by injection, such as parenterally, intravenously, subcutaneously, intramuscularly.

According to one further embodiment, the modified antibody or modified antibody fragment is used in a method of diagnosing, monitoring e.g. monitoring the effectiveness of a treatment e.g. over time, imaging and/or treating a cancer, and is administered concurrently with one or more other therapeutic agents such as chemotherapeutic agents, radiation therapy, immunotherapy agents, autoimmune disorder agents, anti-infectious agents, or one or more other modified antibodies or modified antibody fragments. It is also possible to administer the other therapeutic agent before or after the modified antibody or modified antibody fragment.

### 8. Preparation of the Compounds of the Invention

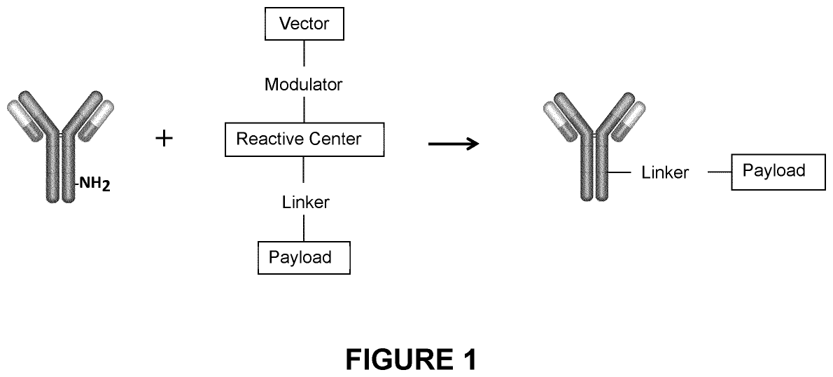

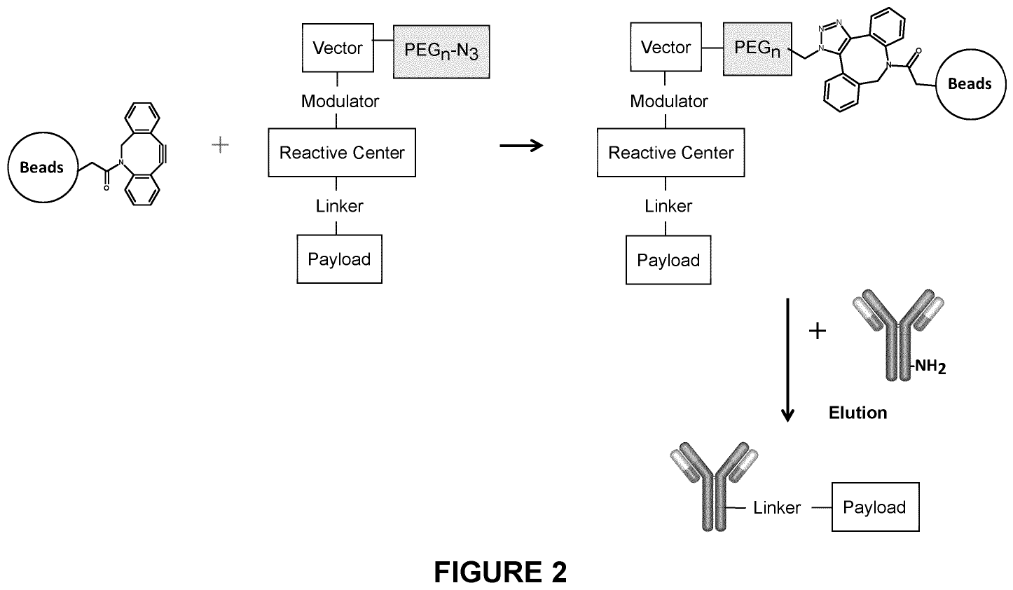

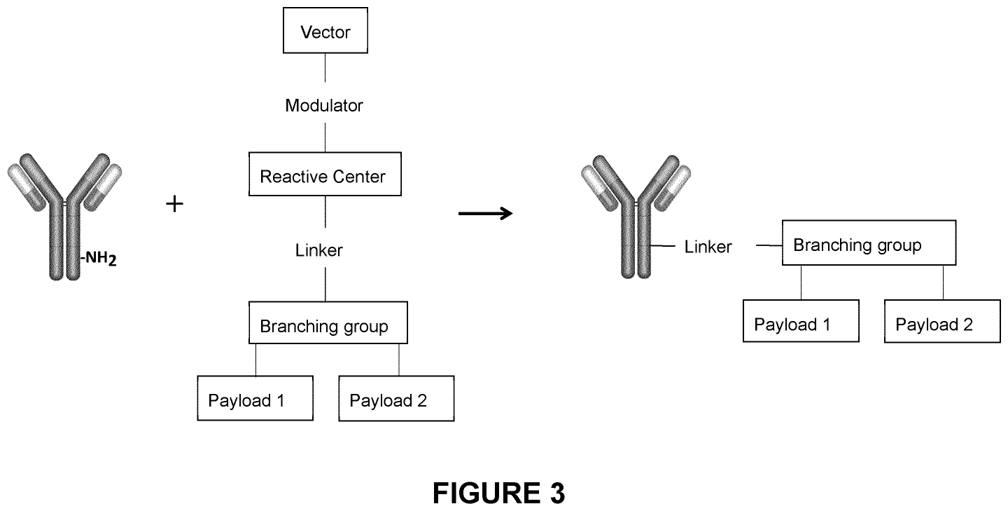

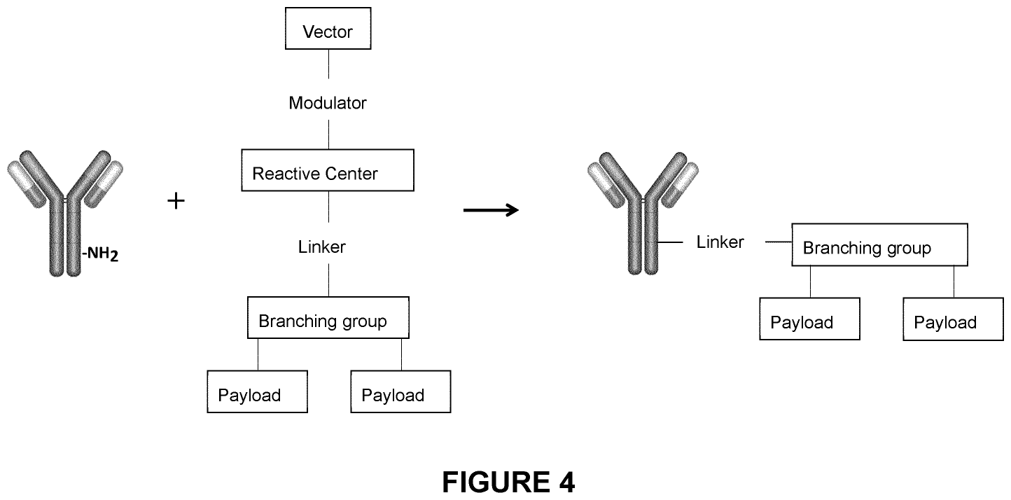

In the following, methods are provided for the preparation of vectors (ligands), spacers, payload-containing groups, and compounds (reactive conjugates), as well as for their use in the regioselective modification of therapeutic antibodies or therapeutic proteins (e.g. Fc-fusion proteins). The compounds of the present invention can be synthesized relying on standard chemical methods and Fmoc-based solid-phase peptide synthesis (SPPS), including on-resin peptide coupling and convergent strategies. The introduction of various payloads, as well as compound immobilization on a solid phase matrix, are also exemplified below. General strategies and methodologies which can be used for the regioselective modification of therapeutic antibodies, or therapeutic proteins (e.g. Fc-fusion proteins), using the compounds of the present invention are known to the skilled person and illustrated in FIGS. 1-4.

### 9. Examples

### 9.1 List of Abbreviations Used in the Examples

Ac: Acetyl
ACN: acetonitrile
BCN: bicyclo[6.1.0]nonyne
Bn: benzyl
Boc: tert-butyloxycarbonyl
CV: column volume
DBCO: Dibenzocyclooctyne
DCC: N,N′-dicyclohexylcarbodiimide
DCM: dichloromethane
DFO: desferrioxamine B
DIC: diisopropylcarbodiimide
DIEA: diisopropylethylamine
DM1: Mertansine
DM4: Ravtansine
DMF: dimethyl formamide
DMSO: dimethyl sulfoxide
DOTA: 1,4,7,10-Tetraazacyclododecane-1,4,7,10-tetraacetic acid
DOTA-GA: 2-(4,7,10-tris(carboxymethyl)-1,4,7,10-tetraazacyclododecan-1-yl)pentanedioic acid
DTPA: diethylenetriaminepentaacetic acid
EA: ethyl acetate
EDC: 1-Ethyl-3-(3-dimethylaminopropyl)carbodiimide
eq.: equivalent
FAM: 5(6)-carboxyfluorescein
FITC: fluorescein isothiocyanate
Fmoc: fluorenylmethoxycarbonyl
HATU·HPF₆: 1-[Bis(dimethylamino)methylene]-1H-1,2,3-triazolo[4,5-b]pyridinium 3-oxide hexafluorophosphate
HPLC: high-performance liquid chromatography
HRMS: high resolution mass spectrometry
Hz: hertz
ma: maleimidoacetic acid
min: minute
Mmt: Monomethoxytrityl
mol: molar
MS: mass spectroscopy
NHS: N-hydroxysuccinimide
NMR: nuclear magnetic resonance
NODA-GA: 2-(4,7-bis(carboxymethyl)-1,4,7-triazonan-1-yl)pentanedioic acid
NOTA: 2,2′,2″-(1,4,7-triazonane-1,4,7-triyl)triacetic acid
PBS: phosphate-buffered saline
PCTA: 2,2′,2″-(3,6,9-triaza-1(2,6)-pyridinacyclodecaphane-3,6,9-triyl)triacetic acid
PEG: polyethylene glycol
pH: potential for hydrogen
Poc: Propargyloxycarbonyl
rt: room temperature
Rt: retention time
SMCC: Succinimidyl 4-(N-maleimidomethyl)cyclohexane-1-carboxylate
SPPS: solid-phase peptide synthesis
TCO: trans-cyclooctene
TEA: triethylamine
TFA: trifluoroacetic acid
THPTA: Tris(3-hydroxypropyltriazolylmethyl)amine
TIS: triisopropylsilane
Tz: tetrazine
UPLC: ultra-performance liquid chromatography
UV: ultraviolet

### 9.2 Starting Materials and Chemicals

The main starting materials and chemicals used in the following examples are listed below:
Fmoc-Rink Amide AM resin, N,N-diisopropylcarbodiimide (DIC), piperazine from Novabiochem (Switzerland) unless indicated otherwise;
Solvents for synthesis, deprotection reagents, cleavage reagents from Merck or Fischer Scientific AG (Switzerland);
TFA, TIS, DIEA, N-hydroxysuccinimide, tri(ethylene glycol) bis(chloroformate), NaN₃, DBU, Boc₂O, DCC, HF·Pyr, 2-iminothiolane hydrochloride, N-hydroxymaleimide, 4-(dimethylamino)pyridine, pyridine, piperidine, dihydrofuran-2,5-dione, copper iodide from Sigma-Aldrich (Switzerland);
Amino acids from Bachem AG (Switzerland), Novabiochem and Aapptec (USA);
Fmoc-L-HTyr(tBu)-OH, Fmoc-VC-PAB-OPNP, THPTA from Iris Biotech GmbH (Germany);
Solvents and chemicals for high-performance liquid chromatography (HPLC), ultra-performance liquid chromatography mass spectrometry (UPLC-MS) from Macherey-Nagel (Switzerland);
2-Aminoethyl-mono-amide-DOTA-tris(t-Bu ester), p-SCN-Bn-CHX-A″-DTPA.3HCl, p-SCN-Bn-PCTA.3HCl, NOTA-Bn-NCS, ma-DFO, DBCO-DFO, p-SCN-Bn-TCMC from Macrocyclics (USA);
Fmoc-N-amido-PEG_{1/2/3/10}-acids, biotin-PEG₁₂-NHS ester, azido-PEG₁₂-NHS ester, DBCO-NHS ester, TCO-NHS ester, Tz-PEG₅-NHS ester, N₃—PEG₁₁-NH₂, Fmoc-N-amido-PEG_{20/24}-acids, m-PEG₂₄-acid, m-PEG₃₇-NHS ester, 3-(3-(tert-butoxy)-3-oxopropoxy)propanoic acid, 2,5-dioxopyrrolidin-1-yl 2-azidoacetate from BroadPharm (USA);
2,5-Dioxopyrrolidin-1-yl3-(2-(2-(3-(2,5-dioxo-2h-pyrrol-1(5h)-yl)propanamido)ethoxy)ethoxy)propanoate, tert-butyl 4-hydroxybenzoate, bis(2,5-dioxopyrrolidin-1-yl) carbonate, 2-azidoethanol from Fluorochem (USA);
tert-butyl 4-hydroxybenzoate, bis(perfluorophenyl) carbonate, HATU from Combi-Blocks (USA);
DFO-SCN, DOTA-NHS ester, tert-butyl 2-[4,10-bis(2-tert-butoxy-2-oxo-ethyl)-7-[2-(2,5-dioxopyrrolidin-1-yl)oxy-2-oxo-ethyl]-1,4,7,10-tetrazacyclododec-1-yl]acetate; hexafluorophosphate, p-SCN-Bn-NODA-GA, DOTA-GA(tBu)₄, NODA-GA-NHS ester from Chematech (France);
ma-template.3HCl, H-Cit-ε-azido-Nle-Tyr(tBu)-OtBu·HCl, 1-hydroxypyrrolidine-2,5-dione, ma-PEG₅-(Lys(Poc)-PEG₅-Arg)-(Lys(Poc)-PEG₁₅-Arg)-Gly-CONH₂*2TFA, H-Cit-ε-azido-Nle-Tyr-OH from Bachem (Switzerland);
DM1-SMCC, Vedotin, MMAE, PNU-159682 from Enovation Chemicals (USA);
3,5-Bis-(((tert-butoxycarbonyl)amino)methyl)benzoic acid from Activate Scientific GmbH (Germany);
EDC.HCl from Apollo Chemical (USA);
DM1 from Immunogen Inc (USA);
3-(4-mercaptophenyl)propanoic acid, 3-(2-((tert-butoxycarbonyl)amino)ethoxy)propanoic acid, 5-FITC-NHS ester from Fluorochem (UK);
DM4 from Carbosynth (USA);
SPDB from MedChemExpress (USA);
AcBr, 3-(4-hydroxyphenyl)propanoic acid from Acros Organics (Belgium);
3-(((4-methoxyphenyl)diphenylmethyl)thio)propanoic acid from LabNetworkCompounds (China);
Dihydroasparagusic acid from Synthonix (USA);
BCN—NHS ester from Berry & Associates Inc (USA);
Fluorescently labeled peptide Fc-III-FAM from Genscript (USA);
Fc-III—(OtBu)₂-L6Orn, Cit-Lys(PEG₅-ma)-Tyr, compound 025 from Ambiopharm (USA)
Herceptin® (trastuzumab), KADCYLA® (ado-trastuzumab emtansine), Tecentriq® (atezolizumab), RITUXAN® (rituximab) from Roche (Switzerland);
Fc region from BioCell (USA);
KEYTRUDA® (pembrolizumab) from MSD Merck Sharp & Dohme;
ZALTRAP® (Aflibercept) from Sanofi-Avents;
VECTIBIX® (panitumumab) from Amgen;
GingisKhan® and Fabricator® proteases from Genovis (Sweden);
EndoS from New England Biolabs (USA);>
HOBt from Fluka (USA).

Biosimilar monoclonal IgG1 antibodies (trastuzumab, alemtuzumab, bevacizumab, rituximab) were produced by cultivation of recombinant CHO cell lines in Dr. G.

Hagens laboratory at the University of Applied Sciences (HES-SO Valais/Wallis, Switzerland).

GingisKhan is a cysteine protease which site-specifically cleave IgG1 above the hinge, thereby generating two Fab fragments and one Fc fragment. Fabricator is a cysteine protease that site-specifically digest antibodies below the hinge, generating F(ab′)₂ and Fc/2 fragments.

### 9.3 Methods

The following methods were used to evaluate the compounds and conjugates of the present invention:

### 9.3.1 Saturation FP Binding Assays

The saturation fluorescence polarization (FP) measurements were performed on SpectraMax Paradigm Multi-Mode Detection Platform (available from Molecular Devices) in flat-bottom 384-well Corning microplates (Merck KGaA), using excitation and emission wavelengths of 485 nm and 535 nm, respectively. The acquisition time was 700 milliseconds and the read height was 1 mm. All reagents used in the assay were diluted in PBS containing 0.05% Tween 20.

Fluorescently labeled peptide Fc-III-FAM (structure shown below) was mixed with a series of IgG1 dilutions in PBS with 0.05% tween to a final peptide concentration of 5 nM. Samples were incubated at 27° C. for 15 min and the fluorescence anisotropy was measured in triplicate.

Fc-III is a 13-mer cyclic peptide known to bind with high affinity to the Fc region of IgG antibodies (DeLano et al. Science 2000, 287, 1279-1283; Nilsson et al. Protein Eng. 1987, 1, 107-113). The fluorescently labeled peptide Fc-III-FAM was prepared by GenScript® using standard SPPS techniques and convergent strategies.

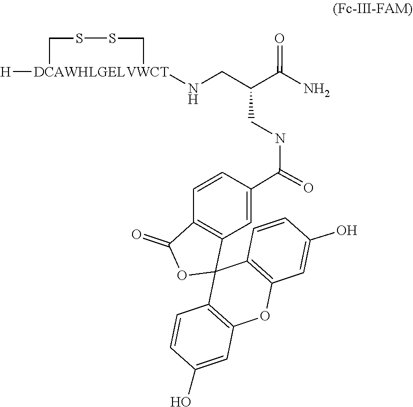

### 9.3.2 Competitive FP Binding Assays

The competitive FP measurements were performed on SpectraMax Paradigm Multi-Mode Detection Platform (Molecular Devices) in flat-bottom 384-well Corning microplates (Merck KGaA), using excitation and emission wavelengths of 485 nm and 535 nm, respectively. The acquisition time was 700 milliseconds and the read height was 1 mm. All reagents used in the assay were diluted in PBS containing 0.05% Tween 20.

Increasing concentrations of the peptide to be measured were mixed with the Fc-III-FAM peptide and added to the IgG1 in a total volume of 80 μL. The final concentration of Fc-III-FAM was kept constant at 5 nM and the final concentration of IgG1 was of 30 nM. The mixture was incubated at 27° C. for 15 min and the fluorescence signal was read on a Spectramax Paradigm. All sample preparations were done in PBS pH 7.0 containing 0.05% Tween. Each experiment was performed in triplicate.

### 9.3.3 Peptide and Conjugate Concentration Determination

Peptide samples were prepared by dissolving the purified peptide, reactive conjugate, or antibody-payload conjugate in DMSO. The concentrations were determined in 1×PBS pH 7.0 using the absorbance of the Trp (E=5500 M⁻¹ cm⁻¹), Tyr (ε=1490 M⁻¹ cm⁻¹), Cys (ε=125 M⁻¹ cm⁻¹) residues, carbonate reactivity modulator (ε=1510 M⁻¹ cm⁻¹), thioester reactivity modulator (ε=1142 M⁻¹ cm⁻¹), p-SCN-Bn-CHX-A″-DTPA (ε=13775 M⁻¹ cm⁻¹), p-SCN-Bn-PCTA (ε=13625 M⁻¹ cm⁻¹), p-SCN-Bn-NOTA (ε=16160 M⁻¹ cm⁻¹), p-SCN-Bn-NODA-GA (ε=14235 M⁻¹ cm⁻¹), DOTA-GA(OtBu)₄—NHS (ε=100 M⁻¹ cm⁻¹), DFO-SCN (ε=21000 M⁻¹ cm⁻¹), DBCO-DFO (ε=9590 M⁻¹ cm⁻¹), DBCO-NHS ester (ε=12560 M⁻¹ cm⁻¹), BCN—NHS (ε=3050 M⁻¹ cm⁻¹), TCO-NHS ester (ε=3300 M⁻¹ cm⁻¹), Tz-PEG₅-NHS ester (ε=16870 M⁻¹ cm⁻¹), Vedotin (ε=1870 M⁻¹ cm⁻¹), SPDB-DM4 (ε=11200 M⁻¹ cm⁻¹), SMCC-DM1 (ε=5700 M⁻¹ cm⁻¹), multi-(DM1)₂ (ε=16332 M⁻¹ cm⁻¹) at 280 nm or the absorbance of FITC (ε=73000 M⁻¹ cm⁻¹), FAM (ε=83000 M⁻¹ cm⁻¹) at 496 nm.

9.3.4 High-resolution mass spectrometry

Deglycosylation of the conjugates was achieved by incubating 1 unit of Endo S per μg of conjugate in the formulation buffer (37° C.-1 hour).

Direct injection HRMS for peptide/conjugate analysis was performed on a QExactive HF Orbitrap-FT-MS, (Thermo Fisher Scientific, Germany) coupled to an automated chip-based nanoelectrospray device (Triversa Nanomate, Advion, USA).

Electrospray ionization was conducted at a capillary voltage of 1.4 kV and nitrogen nanoflow of 0.15 psi. MS experiments were performed with a nominal resolution of 45000 and in the positive ion mode. Data deconvolution was performed with Protein Deconvolution (Thermo Fischer Scientific, USA) using the Xtract algorithm with a 90% fit factor.

For both intact mass measurement (LC-MS) and middle-down analysis (LC-HCDMS/MS), samples were separated onto an Acquity UPLC Protein column BEH C4 (300 Å, 1.7 μm, 1×150 mm, Waters, USA) using a Dionex Ultimate 3000 analytical RSLC system (Dionex, Germany) coupled to a HESI source (Thermo Fisher Scientific, Germany). The separation was performed with a flow rate of 90 μL/min by applying a gradient of solvent B from 15 to 45% in 2 min, then from 45 to 60% within 10 min, followed by column washing and re-equilibration steps. Solvent A was composed of water with 0.1% formic acid, while solvent B consisted of acetonitrile with 0.1% TFA.

Eluting proteoforms were analyzed on a high-resolution QExactive HF-HT-Orbitrap-FTMS benchtop instrument (Thermo Fisher Scientific, Germany). For intact mass measurements MS1, the scan was performed in protein mode with 15000 resolution and averaging 10 pscans. Middle-down analysis for binding site localization was performed in PRM mode isolating species at 1356 m/z for Fc/2-mod, with 300 Th isolation window, 240000 resolution and averaging 10 pscans. HCD (high energy collision-induced dissociation) was used as a fragmentation method with normalized collision energy of 12, 15 and 18%.

Intact mass measurement data were analyzed with Protein Deconvolution (Thermo Fischer Scientific, USA) using a Respect algorithm with 99% noise rejection confidence and 20 ppm accuracy of average mass identification. Middle-down data were deconvoluted using a MASH Suite software (Ge research group, University of Wisconsin). Data obtained with 3 different NCE values were combined together to create a fragmentation map with assigned b- and y-fragments using ProSight Lite software (Kelleher research group, Northwestern University) with 15 ppm mass tolerance.

9.3.5 Determination of the Degree of Conjugation from HRMS Analysis

The average Degree of Conjugation (DoC) values were calculated using the HRMS data and the Equation 1 (Eq. 1) below. These results were derived from the relative peak intensities in deconvoluted mass spectra.

$$D o C = \frac{{\sum }_{k = 0}^{k = n} k * I ( D o {C}_{k} )}{{\sum }_{k = 0}^{k = n} I ( D o {C}_{k} )} \qquad Eq . 1$$

where I(DoCₖ) is the relative peak intensity of conjugates with k add-on molecules per antibody.

### Example 1: Preparation and Characterization of Fc-Binding Vectors

The Fc-binding vectors and vector spacer constructs were prepared using standard Fmoc/tBu-based SPPS, including on-resin coupling and convergent strategies. The ligands prepared in Example 1 are shown in Table 1 below (amino acid substitution numbering refers to the sequence of Fc-III from left to right with N-term D=1 and C-term T=13, e.g. “D1K” indicates that D at position 1 has been replaced by K; bold-underlined indicates that a disulfide bond is present between the side chains of the respective Cys residues).

TABLE 1
Fc-binding vectors

| Compound | Structure |
|---|---|
| Fc-III | H-DCAWHLGELVWCT-NH₂ |
| D1K | Ac-KCAWHLGELVWCT-NH₂ |
| A3K | Ac-DCKWHLGELVWCT-NH₂ |
| W4K | Ac-DCAKHLGELVWCT-NH₂ |
| H5K | Ac-DCAWKLGELVWCT-NH₂ |
| L6K | Ac-DCAWHKGELVWCT-NH₂ |
| G7K | Ac-DCAWHLKELVWCT-NH₂ |
| E8K | Ac-DCAWHLGKLVWCT-NH₂ |
| L9K | Ac-DCAWHLGEKVWCT-NH₂ |
| V10K | Ac-DCAWHLGELKWCT-NH₂ |
| W11K | Ac-DCAWHLGELVKCT-NH₂ |
| T13K | Ac-DCAWHLGELVWCK-NH₂ |

TABLE 2
Fc binding vectors

| Compound | Structure |
|---|---|
| A3Dap | Ac-DCDapWHLGELVWCT-NH₂ |
| A3Dap-PEG₁ | 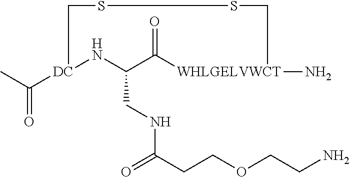 |
| A3Om | Ac-DCOrnWHLGELVWCT-NH₂ |
| A3K-L6Orn | Ac-DCDapWHOrnGELVWCT-NH₂ |
| A3hK | Ac-DChKWHLGELVWCT-NH₂ |
| H5Dap-E8Q | Ac-DCAWDapLGQLVWCT-NH₂ |
| H5Dab | Ac-DCAWDabLGELVWCT-NH₂ |
| H5Dab-E8Q | Ac-DCAWDabLGQLVWCT-NH₂ |
| H5Orn-E8Q | Ac-DCAWOrnLGQLVWCT-NH₂ |
| H5K-E8Q | Ac-DCAWKLGQLVWCT-NH₂ |
| L6Dap | Ac-DCAWHDapGELVWCT-NH₂ |
| L6-(D-Dap) | Ac-DCAWH(D-Dap)GELVWCT-NH₂ |
| L6Dap-E8Q | Ac-DCAWHDapGQLVWCT-NH₂ |
| L6Dab | Ac-DCAWHDabGELVWCT-NH₂ |
| L6Orn | Ac-DCAWHOrnGELVWCT-NH₂ |
| L6-(D-Orn) | Ac-DCAWH(D-Orn)GELVWCT-NH₂ |
| L6-(D-Orn)* | Ac-tcwvleg(D-Orn)hwacd-NH₂ |
| L6Orn-E8Q | Ac-DCAWHOrnGQLVWCT-NH₂ |
| L6K-PEG₁ | 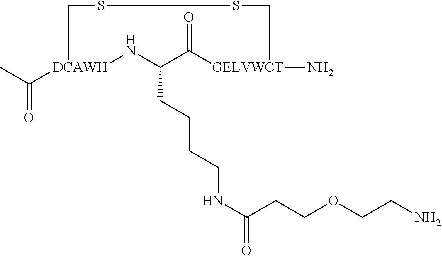 |
| L6K-PEG₂ | 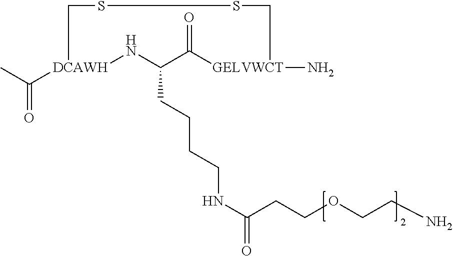 |
| L6K-PEG₃ | 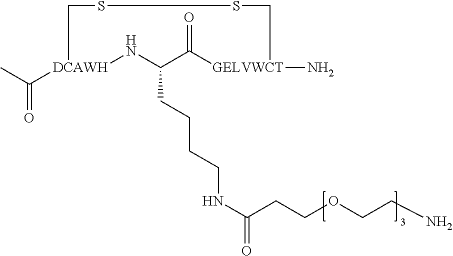 |
| L6K-PEG₁₀ | 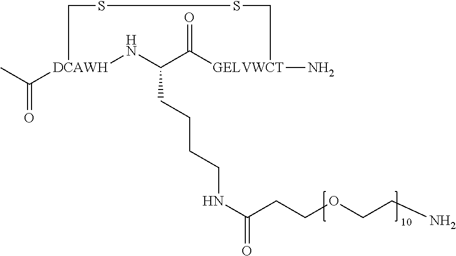 |
| E8Dap | Ac-DCAWHLGDapLVWCT-NH₂ |
| L6Orn-PEG₂₄ | Ac-DCAWHOrnGELVWCT-PEG₂₄-X-NH₂ |
| PEG₂₀-L6Orn | Ac-PEG₂₀-DCAWHOrnGELVWCT-NH₂ |
| PEG₂₄-L6Orn | mPEG₂₄-DCAWHOrnGELVWCT-NH₂ |
| RGNCAYHKGQIIWCTYH** | Ac-RGNCAYHKGQIIWCTYH-NH₂ |
| RGNCAYHOrnGQIIWCTYH** | Ac-RGNCAYHOrnGQIIWCTYH-NH₂ |

*sequence consists of D amino acids
**reference vector
X - —NH(CH₂)(C═O)—
m - methyl

The peptides were prepared by standard Fmoc/tBu-based SPPS using a Rink Amide AM resin (loading: 0.57 mmol/g) and a Liberty Blue™ automated microwave peptide synthesizer (available from CEM Corp., Germany).

Coupling reactions for amide bond formation were performed over 4 min at room temperature using 0.2 M of Fmoc-amino-acids pre-activated with 0.5 M DIC and 1 M OxymaPure® in DMF. Fmoc deprotection was performed with 10% piperazine in DMF (v/v).

After completion of the synthesis, the peptides were cleaved from the resin manually under gentle agitation over 1.5 hour at room temperature by treatment with TFA/TIS/water (90/5/5, v/v/v). After filtration and evaporation of the cleavage mixture with a nitrogen stream, the crude peptides were precipitated with cold diethyl ether, centrifuged, and washed with cold diethyl ether. The peptides were dried, dissolved in ultrapure water/ACN, frozen and lyophilized.

For the synthesis of compounds L6Orn-PEG₂₄, PEG₂₀-L6Orn, and PEG₂₄-L6Orn a solution of Fmoc-N-amido-PEG_{20/24}—COOH or mPEG₂₄-COOH (1.6 eq, 0.16 mmol) and HATU·HPF₆ (1.4 eq, 0.14 mmol) in DMF was stirred for 1 min, and DIEA (6 eq, 0.6 mmol) was added. After 3 min of pre-activation, the peptide on the resin (1 eq, 0.1 mmol, swollen in 3 mL of DMF) was added to the reaction mixture and stirred for 23-27 hours at room temperature. The reaction completion was monitored by ULPC-MS. The resin was washed with DMF and DCM. In the case of L6Orn-PEG₂₄ and PEG₂₀-L6Orn, Fmoc deprotection was performed with 20% piperidine in DMF (v/v) for 30 min at room temperature. The L6Orn-PEG₂₄ synthesis was continued by using Liberty Blue™ automated microwave peptide synthesizer.

For disulfide bond formation, the crude peptides (0.1 mmol) were resuspended in 10 m1 DMSO, then 2 eq of 2 M NH₃ in MeOH and 50 eq of hydrogen peroxide were added and stirred at room temperature for 30 min. The progress of the oxidation was monitored via analytical UPLC-MS. 10 ml of 0.1% TFA aqueous solution was added to the solution to stop the reaction.

The peptides were purified by Preparative Reversed Phase-HPLC on a Kinetex® XB-C18 column (100 Å, 5 μm, 100×21.2 mm; Phenomenex Helvetia) using solvent system A (0.1% TFA in water) and B (0.1% TFA in ACN) at a flow rate of 35 mL/min and a gradient in a range of 15-55% of B over 25 min. Peptide elution was monitored at a wavelength of 214 nm. The appropriate fractions were analyzed by UPLC-MS prior to lyophilization.

For the synthesis of compounds A3Dap-PEG₁, L6K-PEG₁, L6K-PEG₂, L6K-PEG₃ and L6K-PEG₁₀, a solution of Fmoc-NH—(CH₂—CH₂—O)ₙ—CH₂—CH₂—COOH (with n=1, 2, 3, 10; 1.5 eq, 8.2 μmol) and HATU·HPF₆ (1.4 eq, 7.6 μmol) in DMF was stirred for 1 min, and DIEA (2 eq, 10.9 μmol) was added. After 3 min of pre-activation, the Fc-binding peptide (compound L6K) in DMF (1 eq, 5.4 μmol) was added to the reaction mixture and stirred for 1-3 hours at room temperature. Reaction completion was monitored by ULPC-MS. The peptide was then precipitated with cold diethyl ether. Fmoc deprotection was performed with 20% piperidine in DMF (v/v) for 30 min at room temperature, followed by precipitation of the peptide with cold diethyl ether.

The peptides were isolated after HPLC purification (as described in the former paragraph).

The purity of the peptides was determined on UPLC-MS systems:
Method 1: Waters Acquity UPLC system coupled to a Micromass Quattro micro API mass spectrometer with a Kinetex® XB-C18 column (100 Å, 1.7 μm, 50×2.1 mm; Phenomenex Helvetia) using solvent system using solvent system A (0.1% TFA in water) and B (0.1% TFA in ACN) at a flow rate of 0.6 mL/min and a 2-98% gradient of B over 4 min. Peptide elution was monitored at a wavelength of 214 nm.
Method 2: Agilent InfinityLab Liquid Chromatography/Mass Selective Detector XT (LC/MSD XT) system with a Kinetex® XB-C18 column (100 Å, 1.7 μm, 50×2.1 mm; Phenomenex Helvetia) using solvent system using solvent system A (0.03% TFA in water) and B (0.03% TFA in ACN) at a flow rate of 0.62 mL/min and a 5-95% gradient of B over 4 min. Peptide elution was monitored at wavelengths of 214 and 280 nm.
Method 3: Waters Acquity UPLC System coupled to a Waters SQD mass spectrometer, BEH C18 1.7 μm 50×2.1 mm column heated at 40° C. and fitted with 2 μm insert filter pre-columns (available from Waters), and solvent systems A1 (water+0.1% FA) and B1 (ACN+0.1% FA) at a flow rate of 0.9 mL/min and a 5-100% gradient of B1 over 2.9 min.
Method 4: Waters Acquity UPLC System coupled to a Waters SQD mass spectrometer with a CSH C18 column (130 Å, 1.7 μm, 2.1 mm×50 mm) heated at 40° C. using solvent system A (water+0.1% FA) and B (ACN+0.1% FA) at a flow rate of 0.6 mL/min and a 5-85% gradient of B over 5 min.
Method 5: Waters Acquity UPLC System coupled to a Waters SQD mass spectrometer with a CSH C18 column (130 Å, 1.7 μm, 2.1 mm×50 mm) heated at 40° C. using solvent systems A (water+0.1% FA) and B (ACN+0.1% FA) at a flow rate of 0.9 mL/min and a 5-100% gradient of B over 2.7 min.
Method 6: Waters Acquity UPLC System coupled to a Waters SQD mass spectrometer with a CSH Fluoro-phenyl column (130 Å, 1.7 μm, 2.1 mm x 50 mm) heated at 40° C. using solvent system A (water+0.1% FA) and B (ACN+0.1% FA) at a flow rate of 0.9 mL/min and a 5-100% gradient of B over 2.9 min.

The results are shown in the table below.

TABLE 3
Characterization of peptides

| | Cal- | Cal- | | | | |
|---|---|---|---|---|---|---|
| | culated | culated | Found | Retention | | UPLC- |
| | Exact | [M + | [M + | time | Purity | MS |
| Compound | Mass | 2H]²⁺ | 2H]²⁺ | (min) | (%) | method |
| Fc-III | 1529.73 | 765.87 | 765.81 | 1.67 | >95 | 1 |
| D1K | 1584.87 | 793.44 | 793.10 | 1.67 | 98 | 1 |
| A3K | 1628.88 | 815.44 | 815.11 | 1.67 | 99 | 1 |
| W4K | 1513.74 | 757.87 | 757.40 | 2.159 | 99 | 2 |
| H5K | 1562.81 | 782.41 | 782.62 | 1.78 | 98 | 1 |
| L6K | 1586.80 | 794.40 | 794.00 | 2.125 | 100 | 2 |
| G7K | 1642.90 | 822.45 | 822.29 | 1.62 | 86 | 1 |
| E8K | 1570.84 | 786.42 | 786.02 | 1.61 | 99 | 1 |
| L9K | 1586.80 | 794.40 | 794.33 | 1.46 | 99 | 1 |
| V10K | 1600.82 | 801.41 | 801.00 | 2.335 | 100 | 2 |
| W11K | 1513.74 | 757.87 | 757.92 | 1.48 | 98 | 1 |
| T13K | 1598.85 | 800.43 | 799.81 | 1.64 | 99 | 1 |
| A3Dap | 1586.88 | 794.44 | 794.20 | 2.332 | 98 | 2 |
| A3Dap- | 1702.28 | 852.14 | 851.7 | 2.306 | 90 | 2 |
| PEG₁ | | | | | | |
| A3Orn | 1614.88 | 808.44 | 808.10 | 2.31 | 98 | 2 |
| A3K-L6Orn | 1629.89 | 815.95 | 815.40 | 1.952 | 92 | 2 |
| A3hK | 1642.88 | 822.44 | 822.20 | 2.286 | 94 | 2 |
| H5Dap-E8Q | 1519.81 | 760.91 | 760.60 | 2.512 | 99 | 2 |
| H5Dab | 1534.81 | 768.41 | 768.16 | 1.81 | 100 | 1 |
| H5Dab-E8Q | 1533.81 | 767.91 | 767.50 | 2.49 | 99 | 2 |
| H5Orn-E8Q | 1547.81 | 774.91 | 775.16 | 1.76 | 98 | 1 |
| H5K-E8Q | 1561.81 | 781.91 | 781.50 | 2.454 | 99 | 2 |
| L6Dap | 1544.80 | 773.40 | 774.09 | 1.41 | 100 | 1 |
| L6-(D-Dap) | 1544.80 | 773.40 | 773.20 | 2.089 | 95 | 2 |
| L6Dap-E8Q | 1543.81 | 772.91 | 772.50 | 2.078 | 100 | 2 |
| L6Dab | 1558.80 | 780.40 | 780.00 | 2.115 | 90 | 2 |
| L6Orn | 1572.80 | 787.40 | 787.75 | 1.45 | 100 | 1 |
| L6-(D-Orn) | 1572.80 | 787.40 | 787.00 | 2.030 | 94 | 2 |
| L6-(D-Orn)* | 1572.80 | 787.40 | 787.00 | 2.057 | 96 | 2 |
| L6Orn-E8Q | 1571.81 | 786.91 | 786.50 | 2.092 | 54 | 2 |
| L6K-PEG₁ | 1702.23 | 852.12 | 851.60 | 1.47 | 100 | 1 |
| L6K-PEG₂ | 1746.23 | 874.12 | 873.50 | 2.142 | 100 | 2 |
| L6K-PEG₃ | 1790.33 | 896.17 | 895.60 | 1.49 | ND | 1 |
| L6K-PEG₁₀ | 2098.20 | 1050.10 | 1050.00 | 2.238 | 100 | 2 |
| E8Dap | 1528.84 | 765.42 | 765 | 2.267 | 98 | 2 |
| E8Orn | 1556.84 | 779.42 | 779 | 1.949 | 100 | 2 |
| L6Orn- | 2758.39 | 1380.20 | 1379.5 | 2.30 | 98 | 2 |
| PEG₂₄ | | | | | | |
| PEG₂₀- | 2525.94 | 1263.97 | 1263.3 | 1.09 | 88 | 5 |
| L6Orn | | | | | | |
| PEG₂₄- | 2630.06 | 1316.03 | 1315.5 | 2.36 | 98 | 2 |
| L6Orn | | | | | | |
| RGNCAYH | 2089.41 | 1045.71 | 1045.3 | 1.87 | 99 | 2 |
| KGQIIWCT | | | | | | |
| YH | | | | | | |
| RGNCAYH | | | | | | |
| OrnGQIIWC | 2075.41 | 1038.71 | 1038.7 | 1.82 | 98 | 2 |
| TYH | | | | | | |

*sequence consists of D amino acids

### Example 2: Saturation FP Binding Assay

The propensity of the Fc-binding ligand Fc-III-FAM (structure shown above) to bind the Fc region of IgG1 antibodies, i.e. trastuzumab, alemtuzumab, bevacizumab and rituximab, was evaluated in the saturation FP binding assay. It was confirmed that the Fc-binding ligand Fc-III-FAM binds the respective antibodies with high affinity (trastuzumab: 14 nM, alemtuzumab: 13 nm, bevacizumab: 7 nM, rituximab: 11 nM).

### Example 3: Competitive FP Binding Assay

The propensity of the Fc-binding ligands prepared in Example 1 to bind the Fc region of trastuzumab against Fc-III-FAM was evaluated in the competitive FP binding assay described above. The results are given in Table 4 below.

TABLE 4
IC₅₀ values of Fc-binding ligands in competitive FP
binding assay for trastuzumab against Fc-III-FAM

| Compound | IC₅₀ (nM) |
|---|---|
| Fc-III | 161 ± 12 |
| D1K | 672 ± 94 |
| A3K | 1721 ± 263 |
| W4K | 7115 ± 2060 |
| H5K | 1506 ± 92 |
| L6K | 2359 ± 2 |
| G7K | 448 ± 89 |
| E8K | 293 ± 30 |
| L9K | 84 ± 17 |
| V10K | 3272 ± 439 |
| W11K | 12074 ± 944 |
| T13K | 300 ± 81 |

These results confirm that the Fc-binding ligands of Example 1 and Fc-III-FAM compete for the same binding site on the Fc region of trastuzumab. The results demonstrate that lysine mutations of the Fc-III peptide at D1, G7, E8, L9, T13 positions do not significantly alter binding of the mutated peptide to the Fc region of the antibody compared to Fc-III. Mutants A3K, H5K, L6K, and V10K show moderate binding efficiency. However, modification of the Fc-III in compounds W4K and W11K (i.e. replacement of W4 or W11 by lysine) has significant impact on peptide binding to the antibody. Compound W11K was not used in further studies.

### Example 4: Preparation of Building Blocks and Payload-carbonate Derivatives—Compounds 001-016

Compounds 001-027 were prepared according to the procedures described below.

### Preparation of 2-[2-(tert-butoxycarbonylamino)ethoxy]ethyl (4-fluorocarbonylphenyl) carbonate (Compound 001)

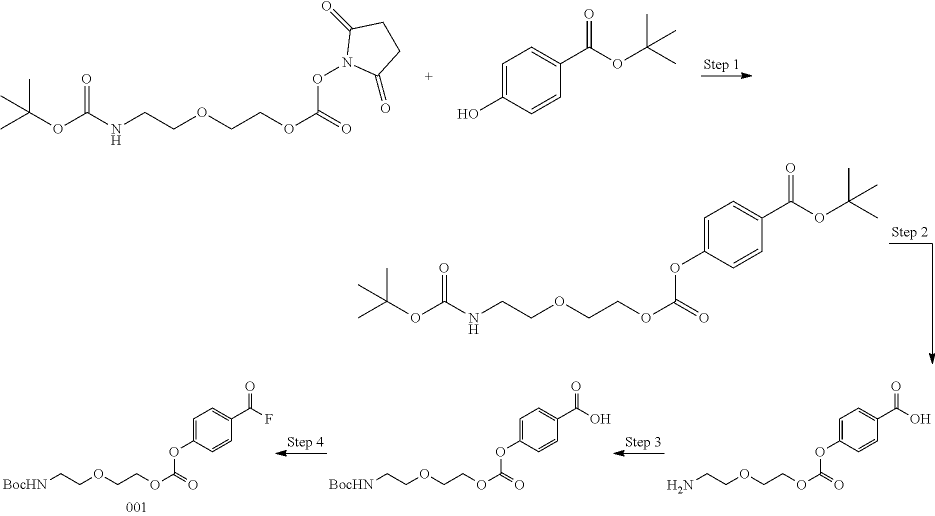 [SMILES: CC(C)(C)OC(=O)NCCOCCOC(=O)ON1C(=O)CCC1=O ; CC(C)(C)OC(=O)c1ccc(O)cc1 ; CC(C)(C)OC(=O)NCCOCCOC(=O)Oc1ccc(C(=O)OC(C)(C)C)cc1 ; CC(C)(C)OC(=O)NCCOCCOC(=O)Oc1ccc(C(=O)F)cc1 ; CC(C)(C)OC(=O)NCCOCCOC(=O)Oc1ccc(C(=O)O)cc1 ; NCCOCCOC(=O)Oc1ccc(C(=O)O)cc1]

Step 1. To a solution of 2-[2-(tert-butoxycarbonylamino)ethoxy]ethyl (2,5-dioxopyrrolidin-1-yl) carbonate (6.61 g, 19.1 mmol, 2.0 eq.) in DCM (70 mL) was added tert-butyl 4-hydroxybenzoate (1.85 g, 9.55 mmol, 1.0 eq.) and then 4-(Dimethylamino)pyridine (2.33 g, 19.1 mmol, 2.0 eq.). The reaction mixture was stirred for 1.5 h at rt then quenched with water. The aqueous phase was extracted three times with DCM. The combined organic layers were dried over MgSO₄ and concentrated in vacuo. Purification by flash chromatography (100 g Cartridge, heptane: EA 4:1 over 3 CV then heptane: EA 4:1 to 1:4 over 9 CV) afforded tert-butyl 4-[2-[2-(tert-butoxycarbonylamino)ethoxy]ethoxycarbonyloxy]benzoate (2.87 g, 6.61 mmol, 69% yield) as a colorless oil. UPLC-MS (method 3): Rt=2.17 min m/z=326 [M-Boc+H]⁺, 448 [M+Na]+. ¹H NMR (400 MHz, DMSO) δ 7.96 (d, 2H), 7.38 (d, 2H), 6.83 (s, 1H), 4.41-4.28 (m, 2H), 3.75-3.60 (m, 2H), 3.43 (t, J=6.0 Hz, 2H), 3.09 (q, 2H), 1.54 (s, 9H), 1.37 (s, 9H).

Step 2. TFA (11 mL) was added to a solution of tert-butyl 4-[2-[2-(tert-butoxycarbonylamino)ethoxy]ethoxycarbonyloxy]benzoate (2.87 g, 5.94 mmol, 1.0 eq.) in DCM (11 mL) a rt. The reaction was stirred at rt for 35 min then concentrated in vacuo and re-evaporated from toluene to afford the corresponding crude TFA salt (3.35 g, quantitative yield assumed). UPLC-MS (method 3): Rt=0.60 min, m/z=270 [M+H]+. ¹H NMR (DMSO) d 8.01 (d, 1H), 7.87 (s, 2H), 7.37 (d, 2H), 4.38 (dd, 2H), 3.75 (dd, 2H), 3.65 (t, 2H), 3.06-2.97 (m, 2H).

Step 3. Crude 4-[2-(2-aminoethoxy)ethoxycarbonyloxy]benzoic acid; 2,2,2-trifluoroacetic acid (1.42 g, 2.22 mmol, 1.0 eq) and by di-tert-butyl dicarbonate (1.50 g, 6.69 mmol, 3 eq.) were dissolved in DCM (24 mL) and DMF (2.4 mL) at rt. Triethylamine (0.6 mL, 4.45 mmol, 2.0 eq.) was added to the reaction mixture at rt. After stirring at rt for 2 h, the reaction mixture was concentrated under vacuum. Purification by flash chromatography (50 g Cartridge, heptane:EA 8:2 to 3:7 over 12 CV followed by 98% EA over 2 CV) afforded 4-[2-[2-(tert-butoxycarbonylamino)ethoxy]ethoxycarbonyloxy]benzoic acid (1.17 g, 3.17 mmol, 70% purity, quantitative yield).

Step 4. Dicyclohexylmethanediimine (158 mg, 0.76 mmol, 1.0 eq.) followed by pyridine hydrofluoride (80 μL, 0.83 mmol, 1.1 eq.) were added to a mixture of 4-[2-[2-(tert-butoxycarbonylamino)ethoxy]ethoxycarbonyloxy]benzoic acid (400 mg, 0.76 mmol, 1.0 eq.) and pyridine (0.15 mL, 1.90 mmol, 2.5 eq.) in DCM (4 mL) in a plastic vessel at rt. After stirring at rt for 2 h, 0.5 equiv. of HF pyridine, 0.5 equiv. of DCC and 1 equiv. of pyridine were added to the reaction mixture. After stirring at rt for 4 h, 0.5 equiv. of HF pyridine, 0.5 equiv. of DCC and 1 equiv. of pyridine were added to the reaction mixture. After stirring at rt for 5 h, the reaction mixture was filtrated through a pad of Celite and concentrated in vacuo. Purification by flash chromatography (25 g Cartridge, heptane:EA 9:1 to 3:7 over 12 CV) afforded 2-[2-(tert-butoxycarbonylamino)ethoxy]ethyl (4-fluorocarbonylphenyl) carbonate (compound 001) (257.6 mg, 0.69 mmol, 92% yield) as a colourless oil. UPLC-MS (method 3): Rt=1.86 min, m/z=272 [M+H]⁺, 394 [M+Na]+; ¹H NMR (400 MHz, CDCl₃) δ 8.05-7.99 (m, 2H), 7.36-7.28 (m, 2H), 4.83 (br s, 1H), 4.40-4.32 (m, 2H), 3.75-3.66 (m, 2H), 3.52 (t, J=5.2 Hz, 2H), 3.28 (q, J=5.1 Hz, 2H), 1.38 (s, 9H).

### Preparation of 3-[4-[3-(2-aminoethoxy)propionylsulfanyl]phenyl]propanoic (Compound 002)

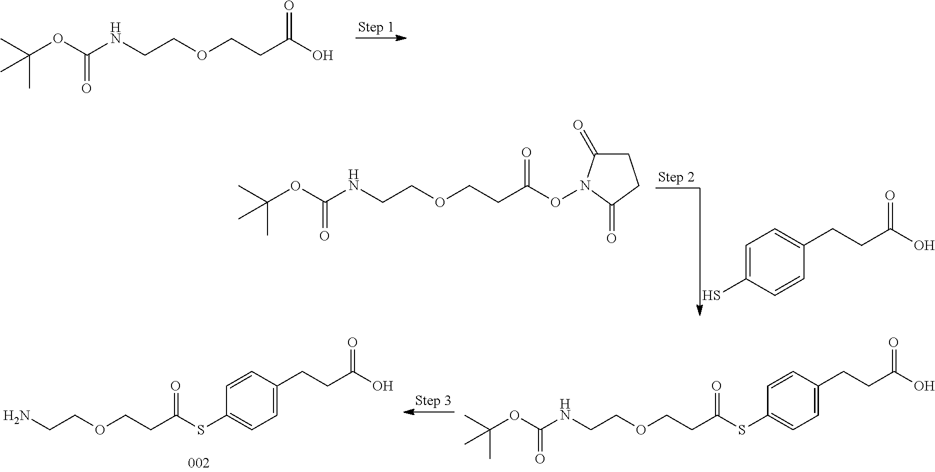 [SMILES: CC(C)(C)OC(=O)NCCOCCC(=O)O ; CC(C)(C)OC(=O)NCCOCCC(=O)ON1C(=O)CCC1=O ; O=C(O)CCc1ccc(S)cc1 ; NCCOCCC(=O)Sc1ccc(CCC(=O)O)cc1 ; CC(C)(C)OC(=O)NCCOCCC(=O)Sc1ccc(CCC(=O)O)cc1]

Step 1. To a solution of 3-(2-((tert-butoxycarbonyl)amino)ethoxy)propanoic acid (1.50 g, 6.24 mmol, 1.0 eq.) in DCM (25 mL) at rt was added EDC.HCl (4.78 g, 24.95 mmol, 4.0 eq.) followed by 1hydroxypyrrolidine-2,5-dione (2.87 g, 24.95 mmol, 4.0 eq.). After stirring at rt for 16 h, purification on C18 (60 g, 20 to 80% of ACN+0.1% TFA in water+0.1% TFA over 10 CV) afforded (2,5-dioxopyrrolidin-1-yl) 3-[2-(tertbutoxycarbonylamino)ethoxy]propanoate (1.77 g, 5.37 mmol, 94% UV purity, 81% yield) as a colourless oil after freeze-drying. UPLC-MS (method 3): Rt=1.26 min, m/z=331 [M+H]*, 232 [M-Boc+H]⁺, 375 [M+FA-H]⁻; ¹H NMR (CDCl₃, 400 MHz): δ 5.24 (br s, 1H), 3.78 (t, J=6.1 Hz, 2H), 3.51 (t, J=5.0 Hz, 2H), 3.36-3.20 (m, 2H), 2.90-2.73 (m, 6H), 1.41 (s, 9H); ¹³C NMR (CDCl₃, 100 MHz): b 169.1, 166.6, 156.3, 79.2, 70.4, 65.6, 40.4, 32.4, 28.5, 25.7.

Step 2. DIEA (0.12 mL, 0.67 mmol, 1.0 eq.) was added to a mixture of (2,5-dioxopyrrolidin-1-yl) 3-[2-(tert-butoxycarbonylamino)ethoxy]propanoate (221 mg, 0.67 mmol, 1.0 eq.) and 3-(4-mercaptophenyl)propanoic acid (128 mg, 0.67 mmol, 1.0 eq.) in DMF (3 mL) at rt. After stirring at rt for 2 days, purification on C18 (30 g, 20 to 80% of ACN+0.1% TFA in water+0.1% TFA over 12 CV) afforded 3[4[3[2(tertbutoxycarbonylamino)ethoxy]propanoylsulfanyl]phenyl]propanoic acid (99.6 mg, 0.24 mmol, 95% UV purity, 36% yield) as a white solid after freeze-drying. UPLC-MS (method 3): Rt=1.67 min, m/z=298 [M-Boc+H]⁺, 396 [M−H]—; ¹H NMR (CDCl₃, 400 MHz): δ 7.36 (d, J=7.9 Hz, 2H), 7.27 (d, J=7.9 Hz, 2H), 3.75 (t, J=6.2, 2H), 3.49 (t, J=5.0, 2H), 3.36-3.24 (m, 2H), 2.99 (t, J=7.6, 2H), 2.88 (t, J=5.7, 2H), 2.69 (t, J=7.6, 2H), 1.44 (s, 9H); ¹³C NMR (CDCl₃, 100 MHz): δ 176.8, 142.1, 134.9, 133.2, 129.4, 125.4, 70.2, 66.3, 43.8, 35.0, 30.4, 28.6.

Step 3. TFA (1 mL) was added to 3-[4-[3-[2-(tert-butoxycarbonylamino)ethoxy]propanoylsulfanyl]phenyl]propanoic acid (75.6 mg, 0.19 mmol, 1.0 eq.) at rt. The reaction mixture was stirred at rt for 10 min then concentrated in vacuo affording 3-[4-[3-(2-aminoethoxy)propanoylsulfanyl]phenyl]propanoic acid (compound 002) (51.4 mg, 0.17 mmol, 98% UV purity, 89% yield) as a white solid. UPLC-MS (method 3): Rt=0.81 min, m/z=299 [M+H]⁺, 296 [M−H]⁻. ¹H NMR (DMSO-d₆, 400 MHz): δ 5.14 (d, J=8.4, 2H), 5.12 (d, J=8.8 Hz, 2H), 1.60 (t, J=5.8 Hz, 2H), 1.47 (t, J=5.1 Hz, 2H), 0.92 (t, J=4.8 Hz, 2H), 0.78 (t, J=5.7 Hz, 2H), 0.72 (t, J=7.4 Hz, 2H), 0.47 (t, J=7.4 Hz, 2H); ¹³C NMR (DMSO-d₆, 100 MHz): b 199.7, 175.5, 140.9, 132.6, 127.4, 121.8, 64.1, 40.6, 36.7, 32.7, 27.7.

### Preparation of 4-[3-[2-(tert-butoxycarbonylamino)ethoxy]propanoyloxy]benzoic acid (Compound 003)

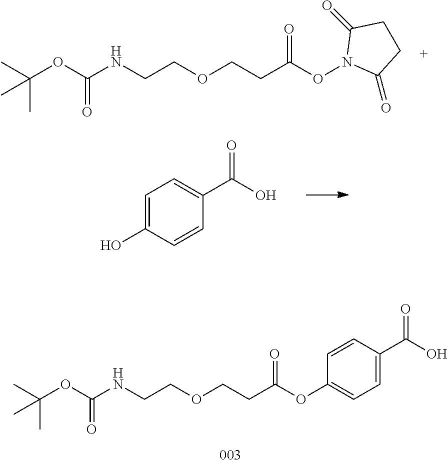 [SMILES: CC(C)(C)OC(=O)NCCOCCC(=O)ON1C(=O)CCC1=O ; O=C(O)c1ccc(O)cc1 ; CC(C)(C)OC(=O)NCCOCCC(=O)Oc1ccc(C(=O)O)cc1]

4-hydroxybenzoic acid (65.4 mg, 0.47 mmol, 1.0 eq.) was added to a mixture of (2,5-dioxopyrrolidin-1-yl) 3-[2-(tert-butoxycarbonylamino)ethoxy]propanoate (158.1 mg, 0.47 mmol, 1.0 eq.) and DBU (0.28 mL, 1.88 mmol, 4.0 eq.) in DMF (1.6 mL) at rt. After stirring at rt for 1 h, purification on C18 (30 g, 20 to 80% of ACN+0.1% TFA in water+0.1% TFA over 10 CV) afforded 4-[3-[2-(tert-butoxycarbonylamino)ethoxy]propanoyloxy]benzoic acid (compound 003) (9.2 mg, 25.0 μmol, 96% UV purity, 5% yield) as a white solid after freeze-drying. UPLC-MS (method 3): Rt=1.46 min, m/z=254 [M+H-Boc]+, 352 [M−H]⁻.

### Preparation of 3-[4-[3-[2-(tert-butoxycarbonylamino)ethoxy]propanoyloxy]phenyl]propanoic acid (Compound 004)

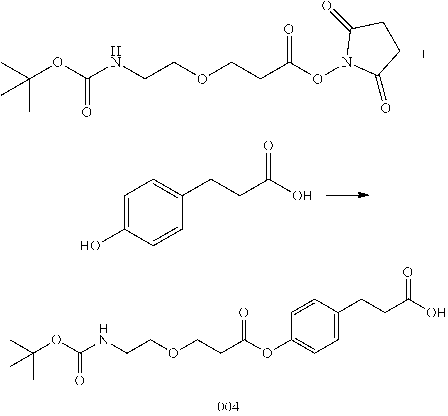 [SMILES: CC(C)(C)OC(=O)NCCOCCC(=O)ON1C(=O)CCC1=O ; O=C(O)CCc1ccc(O)cc1 ; CC(C)(C)OC(=O)NCCOCCC(=O)Oc1ccc(CCC(=O)O)cc1]

3-(4-hydroxyphenyl)propanoic acid (160.5 mg, 0.96 mmol, 1.0 eq.) was added to a mixture of (2,5-dioxopyrrolidin-1-yl) 3-[2-(tert-butoxycarbonylamino)ethoxy]propanoate (322.4 mg, 0.96 mmol, 1.0 eq.) and DIEA (0.17 mL, 0.96 mmol, 1.0 eq.) in DMF (4 mL) at rt. After stirring at rt for 16 h, purification on 018 (30 g, 20 to 80% of ACN+0.1% TFA in water+0.1% TFA over 12 CV) afforded 3-[4-[3-[2-(tert-butoxycarbonylamino)ethoxy]propanoyloxy]phenyl]propanoic acid (compound 004) (15.1 mg, 39.6 μmol, 100% UV purity, 4% yield) as a white solid after freeze-drying. UPLC-MS (method 4): Rt=1.55 min, m/z=282 [M+H-Boc]*, 380 [M−H]⁻.

### Preparation of 3-(((4-methoxyphenyl)diphenylmethyl)thio)propanoic acid (Compound 005)

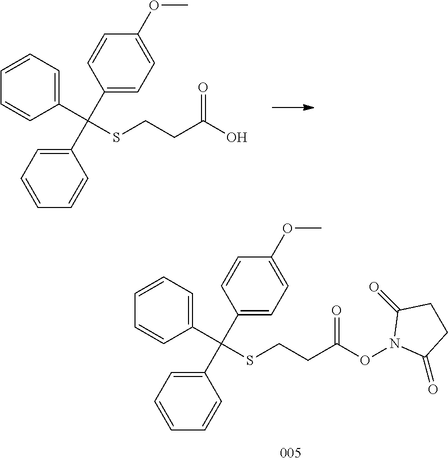 [SMILES: COc1ccc(C(SCCC(=O)O)(c2ccccc2)c2ccccc2)cc1 ; COc1ccc(C(SCCC(=O)ON2C(=O)CCC2=O)(c2ccccc2)c2ccccc2)cc1]

EDC·HCl (289 mg, 1.51 mmol, 3.0 eq.) and 1-hydroxypyrrolidine-2,5-dione (173 mg, 1.51 mmol, 3.0 eq.) were added to a solution of 3-(((4-methoxyphenyl)diphenylmethyl)thio)propanoic acid (200 mg, 0.50 mmol, 1.0 eq.) in DCM (6 mL) at rt. After stirring at rt for 2 h, purification by silica gel flash chromatography (25 g, 20 to 80% of EA in cyclohexane over 12 CV) afforded compound 005 (202 mg, 0.42 mmol, 85% yield) as a white solid after concentration in vacuo. ¹H NMR (CDCl₃, 400 MHz): δ 7.47-7.39 (m, 4H), 7.37-7.32 (m, 2H), 7.32-7.24 (m, 4H), 7.24-7.17 (m, 2H), 6.86-6.79 (m, 2H), 3.78 (s, 3H), 2.76 (br s, 4H), 2.55 (t, J=7.4 Hz, 2H), 2.42 (t, J=7.4, 2H); ¹³C NMR (CDCl₃, 100 MHz): b 169.0, 167.2, 158.3, 144.7, 136.5, 130.8, 129.5, 128.1, 126.9, 113.4, 66.7, 55.3, 30.6, 26.2, 25.6.

### Preparation of (4-[2-[2-[[2-[4,7,10-tris(2-tert-butoxy-2-oxo-ethyl)-1,4,7,10-tetrazacyclododec-1-yl]acetyl]amino]ethoxy]ethoxycarbonyloxy]benzoic acid) (Compound 006)

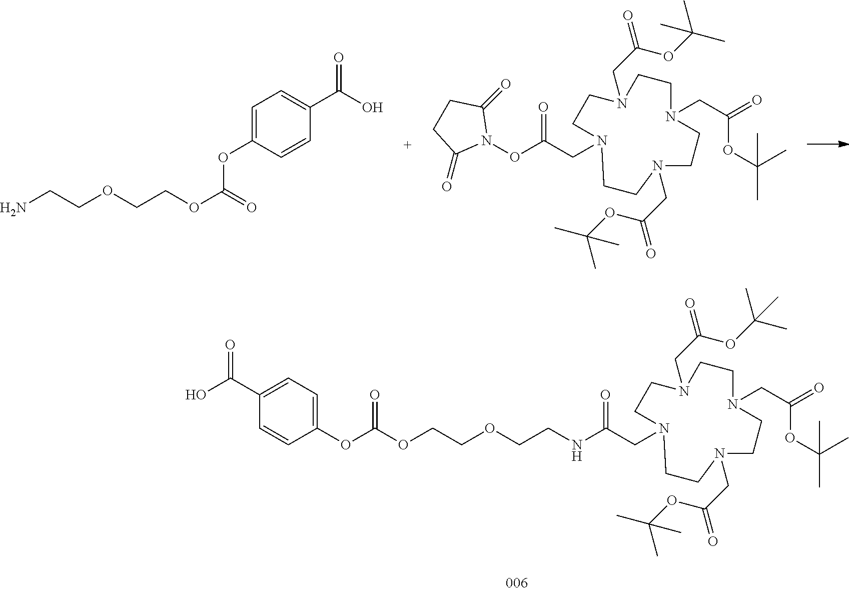

To a solution of 0.70 g of DOTA-tris(tBu)ester NHS ester (0.83 mmol) in 3.5 mL of ACN were added 0.88 mL of DIEA (5.0 mmol, 6.0 eq) then 0.44 g of the TFA salt of 4-[2-(2-aminoethoxy)ethoxycarbonyloxy]benzoic acid (0.92 mmol, 1.1 eq) and the reaction mixture was stirred at room temperature for 10 min (a solid appeared immediately which was solubilized after sonification). The solution was diluted in 3.5 mL of water and purified by C18 cartridge Flash Chromatography (water/ACN, 90/10 to 0/100). Fractions were gathered, concentrated in vacuo and lyophilized to afford 0.66 g of compound 006 (4-[2-[2-[[2-[4,7,10-tris(2-tert-butoxy-2-oxo-ethyl)-1,4,7,10-tetrazacyclododec-1-yl]acetyl]amino]ethoxy]ethoxycarbonyloxy]benzoic acid) as a white solid (Purity>95%, Yield: 93%). LCMS: m/z=824 [M+H]⁺, 413 [M/2+H]+. ¹H NMR (DMSO) δ 8.56 (s, 1H), 7.95 (d, 2H), 7.27 (d, 2H), 4.31 (s, 2H), 3.66 (s, 2H), 3.48-3.42 (m, 2H), 3.35-3.25 (m, 8H), 3.00 (s, 2H), 2.75 (s, 8H), 2.63 (s, 4H), 1.37 (s, 27H).

### Preparation of Compound 3-[4-[2-[2-[[2-[4,7,10-tris(2-tert-butoxy-2-oxo-ethyl)-1,4,7,10-tetrazacyclododec-1-yl]acetyl]amino]ethoxy]ethoxycarbonyloxy]phenyl]propanoic acid (Compound 007)

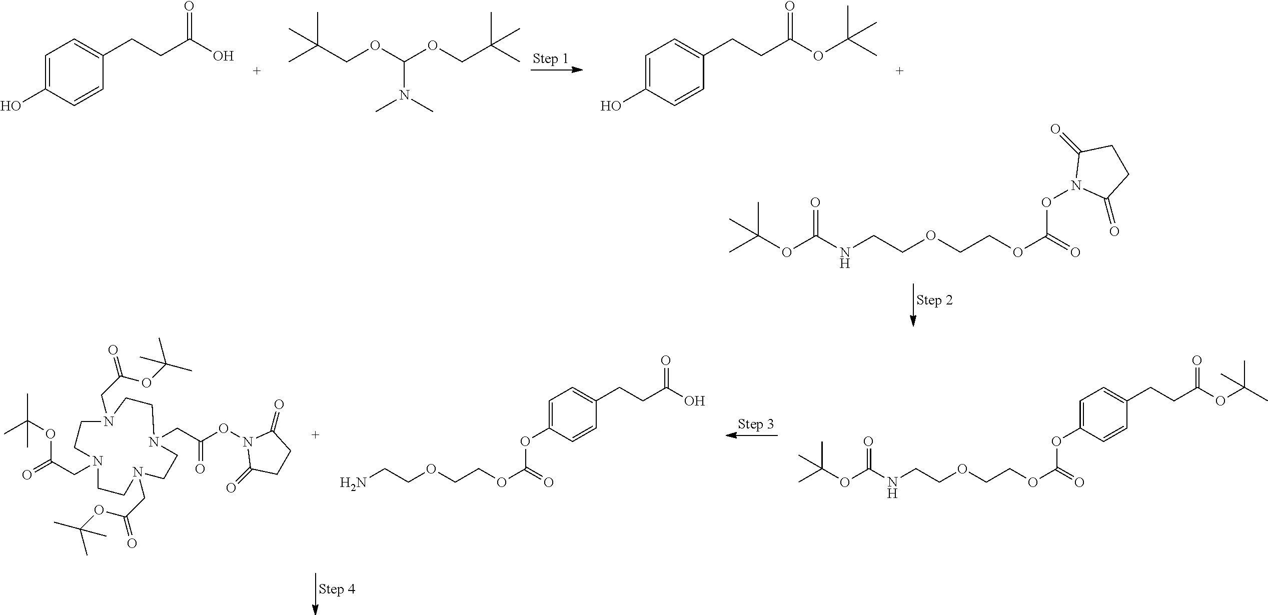

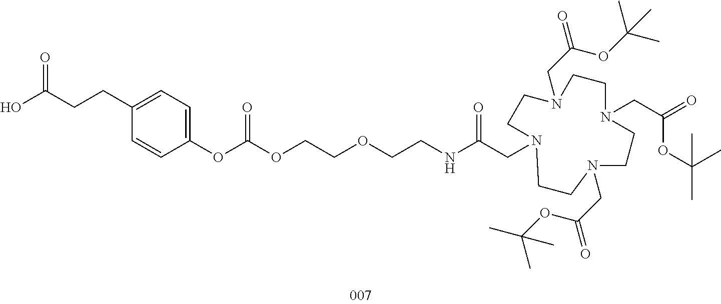

Step 1. To a solution of 0.70 g of 3-(4-hydroxyphenyl)propanoic acid (4.1 mmol) in 11 mL of toluene was added 4.6 g of 2-methylpropan-2-ol (62 mmol, 15 eq.) and the reaction mixture was heated at 85° C. 4.7 mL of N,N-dimethylformamide dineopentyl acetal (17 mmol, 4.0 eq.) was slowly added and the reaction mixture was stirred at 85° C. for 6 h. 50 mL of an aqueous solution of saturated NaHCO₃ was added and the aqueous layer was extracted with 3×10 mL of EA. Combined organic layers were washed with 10 mL of water and concentrated under vacuum to yield 0.74 g of tert-butyl 3-(4-hydroxyphenyl)propanoate as clear pink oil which was used for next step without further purification (83% UV purity, 67% yield). LCMS: m/z=221 [M−H]⁻.

Step 2. A solution of 0.35 g of tert-butyl 3-(4-hydroxyphenyl)propanoate (1.3 mmol) in 11 mL of DCM was added on 0.91 g of compound 2 (2.6 mmol, 2.0 eq.) then 0.32 g of 4-(dimethylamino)pyridine (2.6 mmol, 2.0 eq.). The reaction mixture was stirred at room temperature for 2 h. 50 mL of water was added and the aqueous layer was extracted with 3×10 mL of DCM. The combined organic layers were concentrated under vacuum and the residue was purified by Flash Chromatography (cyclohexane/ethyl acetate 90/10 to 60/40) to yield 0.55 g of tert-butyl 3-[4-[2-[2-(tert-butoxycarbonylamino)ethoxy]ethoxycarbonyloxy]phenyl]propanoate as a colorless oil (97% UV purity, 90% yield). LCMS: m/z=298 [M-Boc-(t-Bu)+H]⁺, 341 [M-2(t-Bu)+H]⁺, 354 [M-Boc+H]⁺, 397 [M-(t-Bu)+H]⁺, 476 [M+Na]+. ¹H NMR (DMSO): δ 7.27 (d, 2H), 7.12 (d, 2H), 6.83 (t, 1H), 4.35-4.24 (m, 2H), 3.65 (dd, 2H), 3.42 (t, 2H), 3.08 (q, 2H), 2.81 (t, 2H), 1.37 (s, 9H), 1.35 (s, 9H).

Step 3. To a solution of 1.7 mL of TFA (22 mmol, 20 eq.) in 5.1 mL of DCM was added 0.52 g of tert-butyl 3-[4-[2-[2-(tert-butoxycarbonylamino)ethoxy]ethoxycarbonyloxy]phenyl]propanoate (1.1 mmol) at 0° C. and the reaction mixture was stirred at room temperature for 3 h. DCM and TFA were evaporated under vacuum to yield 0.63 g of 3-[4-[2-(2-aminoethoxy)ethoxycarbonyloxy]phenyl]propanoic acid; 2,2,2-trifluoroacetic acid as a colorless oil which was used for next step without further purification (70% UV purity, quantitative yield). LCMS: m/z=296 [M−H]⁻, 298 [M+H]+. ¹H NMR (DMSO): δ 7.84 (s, 3H), 7.28 (d, 2H), 7.12 (d, 2H), 4.38-4.32 (m, 2H), 3.78-3.71 (m, 2H), 3.64 (t, 2H), 3.01 (q, 2H), 2.83 (t, 2H).

Step 4. To a solution of 0.54 g of 3-[4-[2-(2-aminoethoxy)ethoxycarbonyloxy]phenyl]propanoic acid; 2,2,2-trifluoroacetic acid (0.92 mmol, 1.1 eq.) in 3.5 mL of acetonitrile were added 0.70 g of tri-tert-butyl 2,2′,2″-(10-(2-((2,5-dioxopyrrolidin-1-yl)oxy)-2-oxoethyl)-1,4,7,10-tetraazacyclododecane-1,4,7-triyl)triacetate (0.83 mmol) followed by 0.88 mL of N,N-diisopropylethylamine (5.0 mmol, 6.0 eq.) and the reaction mixture was stirred at room temperature for 10 min. 3.0 mL of water was added and the solution was purified by C18 Flash Chromatography (water/acetonitrile 95/5 to 0/1) to yield 0.62 mg of compound 007 (3-[4-[2-[2-[[2-[4,7,10-tris(2-tert-butoxy-2-oxo-ethyl)-1,4,7,10-tetrazacyclododec-1-yl]acetyl]amino]ethoxy]ethoxycarbonyloxy]phenyl]propanoic acid) as a white solid (Purity=98%, Yield: 85%). LCMS: m/z=342.7 [(M-3(t-Bu))/2+H]⁺, 370.8 [(M-2(t-Bu))/2+H]⁺, 398.9 [(M-(t-Bu))/2+H]⁺, 427 [M/2+H]⁺, 852 [M+H]+. ¹H NMR (DMSO): δ 8.56 (t, 1H), 7.27 (d, 2H), 7.10 (d, 2H), 4.33-4.25 (m, 2H), 3.69-3.65 (m, 2H), 2.93 (s, 2H), 2.85-2.70 (m, 10H), 2.68-2.59 (m, 4H), 1.41 (s, 27H).

### Preparation of Compound 3-(4-((3-(2-(2-(4,7,10-tris(2-(tert-butoxy)-2-oxoethyl)-1,4,7,10-tetraazacyclododecan-1-yl)acetamido)ethoxy)propanoyl)thio)phenyl)propanoic acid (Compound 008)

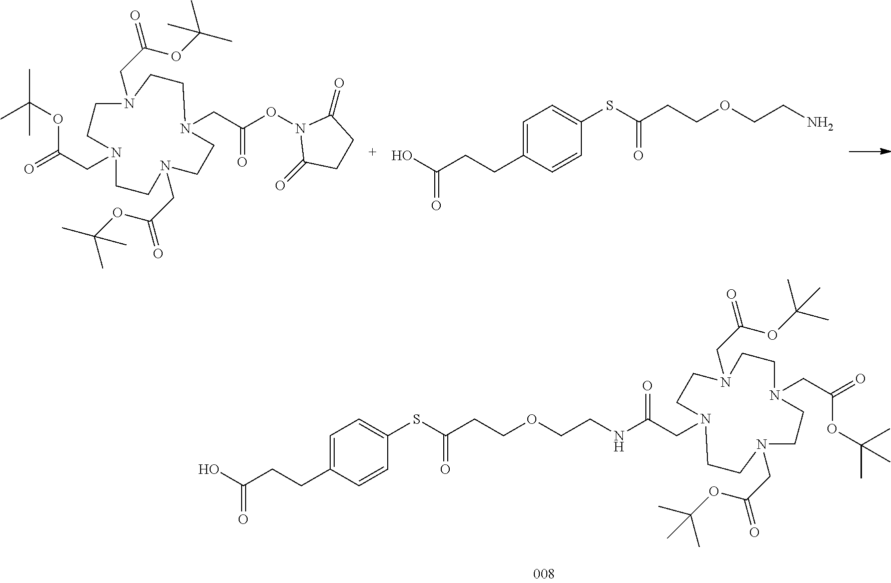 [SMILES: CC(C)(C)OC(=O)CN1CCN(CC(=O)ON2C(=O)CCC2=O)CCN(CC(=O)OC(C)(C)C)CCN(CC(=O)OC(C)(C)C)CC1 ; NCCOCCC(=O)Sc1ccc(CCC(=O)O)cc1 ; CC(C)(C)OC(=O)CN1CCN(CC(=O)NCCOCCC(=O)Sc2ccc(CCC(=O)O)cc2)CCN(CC(=O)OC(C)(C)C)CCN(CC(=O)OC(C)(C)C)CC1]

tert-butyl 2-[4,10-bis(2-tert-butoxy-2-oxo-ethyl)-7-[2-(2,5-dioxopyrrolidin-1-yl)oxy-2-oxo-ethyl]-1,4,7,10-tetrazacyclododec-1-yl]acetate; hexafluorophosphate (103.3 mg, 0.13 mmol, 1.0 eq.) followed by DIEA (90 μL, 0.51 mmol, 4.0 eq.) were added to a solution of 3-[4-[3-(2-aminoethoxy)propanoylsulfanyl]phenyl]propanoic acid (37.7 mg, 0.13 mmol, 1.0 eq.) in DMF (0.6 mL) at rt. After stirring at rt for 1.5 h, TFA was added until acidic pH was reached. Purification by preparative HPLC (5-50% ACN+0.1% FA in water+0.1% FA) was achieved yielding compound 008 (3-(4-((3-(2-(2-(4,7,10-tris(2-(tert-butoxy)-2-oxoethyl)-1,4,7,10-tetraazacyclododecan-1-yl)acetamido)ethoxy)propanoyl)thio)phenyl)propanoic acid) (64.3 mg, 60.4 mmol, 80% UV purity, 48% yield) as white powder after freeze-drying. UPLC-MS (method 4): Rt=2.61 min, m/z=852 [M+H]*, 850 [M−H]⁻.

### Preparation of Compound 4-[2-[2-[(3′,6′-dihydroxy-3-oxo-spiro [isobenzofuran-1,9′-xanthene]-5-yl)carbamothioylamino]ethoxy]ethoxycarbonyloxy]benzoic acid (Compound 009)

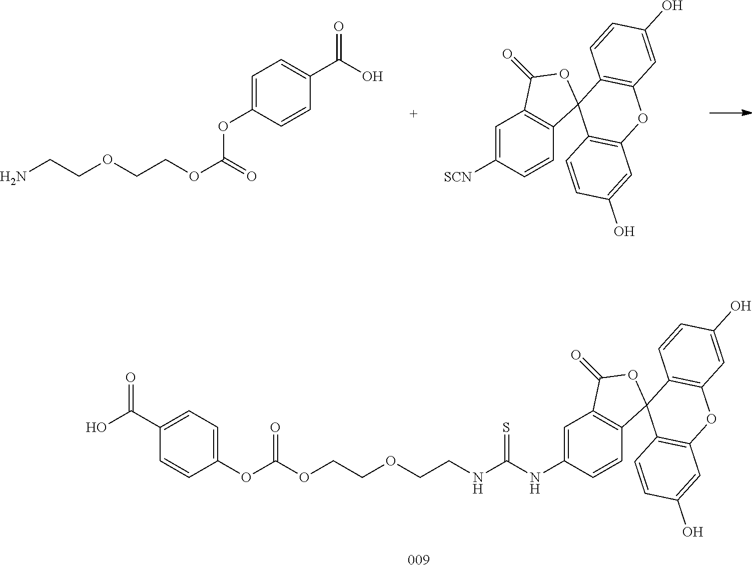

To a solution of 0.71 g of 4-[2-(2-aminoethoxy)ethoxycarbonyloxy]benzoic acid; 2,2,2-trifluoroacetic acid (1.2 mmol, 1.2 eq.) in 4.0 mL of ACN were added 0.40 g of fluorescein isothiocyanate isomer (1.0 mmol) and 4.0 mL of DMF followed by 1.1 mL of N,N-diisopropylethylamine (6.0 mmol, 6.0 eq.). This mixture was stirred for 10 min at room temperature. Solvents were evaporated under vacuum. The residue was purified by C18 Flash Chromatography (water/acetonitrile 95/5 to 0/1) to yield 0.38 g of compound 009 (4-[2-[2-[(3′,6′-dihydroxy-3-oxo-spiro [isobenzofuran-1,9′-xanthene]-5-yl)carbamothioylamino]ethoxy] ethoxycarbonyloxy] benzoic acid) as an orange solid (Purity: 98%, Yield: 56%). LCMS: m/z=657 [M−H]⁻, 659 [M+H]+. ¹H NMR (DMSO): δ 13.07 (s, 1H), 10.24-9.95 (m, 3H), 8.26 (s, 1H), 8.16 (s, 1H), 7.98 (d, 2H), 7.74 (d, 1H), 7.35 (d, 2H), 7.18 (d, 1H), 6.67 (d, 2H), 6.61-6.53 (m, 4H), 4.42-4.37 (m, 2H), 3.80-3.66 (m, 6H).

### Preparation of tri-tert-butyl 2,2′,2″-(10-(1-((2,5-dioxopyrrolidin-1-yl)oxy)-1,12,17-trioxo-2,5,8,11-tetraoxa-13,16-diazaoctadecan-18-yl)-1,4,7,10-tetraazacyclododecane-1,4,7-triyl)triacetate (Compound 010)

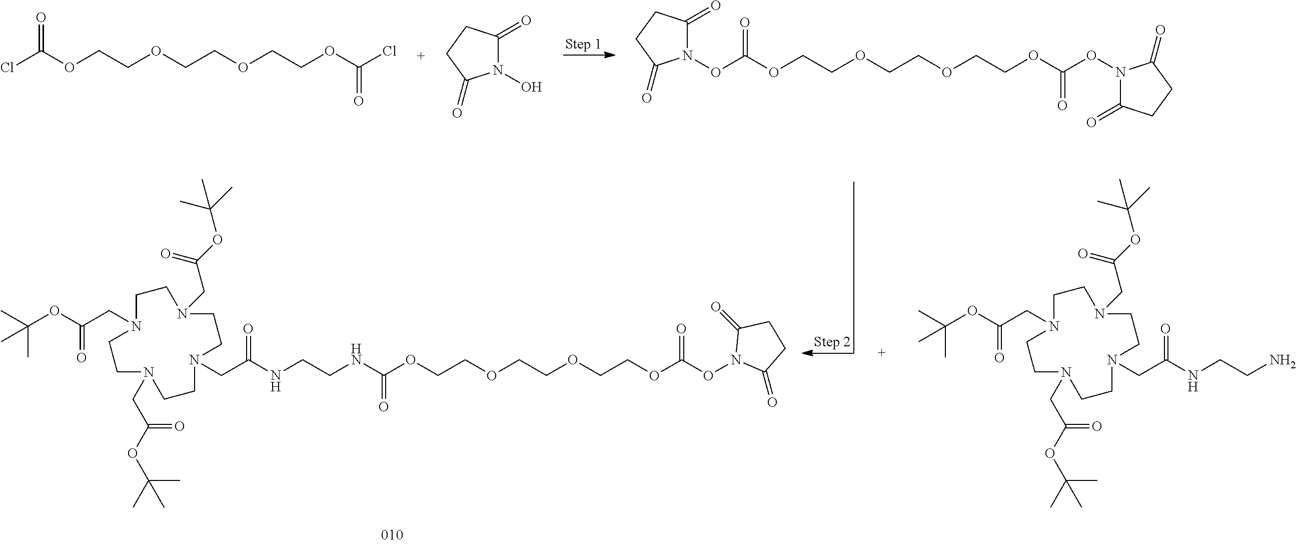 [SMILES: O=C(Cl)OCCOCCOCCOC(=O)Cl ; O=C1CCC(=O)N1O ; O=C(OCCOCCOCCOC(=O)ON1C(=O)CCC1=O)ON1C(=O)CCC1=O ; CC(C)(C)OC(=O)CN1CCN(CC(=O)NCCNC(=O)OCCOCCOCCOC(=O)ON2C(=O)CCC2=O)CCN(CC(=O)OC(C)(C)C)CCN(CC(=O)OC(C)(C)C)CC1 ; CC(C)(C)OC(=O)CN1CCN(CC(=O)NCCN)CCN(CC(=O)OC(C)(C)C)CCN(CC(=O)OC(C)(C)C)CC1]

Step 1. To a stirring solution of N-hydroxysuccinimide (1.29 g, 11 mmol) in THF (20 mL) was added tri(ethylene glycol) bis(chloroformate) (0.5 g, 1.8 mmol), and the solution was cooled in an ice bath. Triethylamine (1.866 mL, 1.41 g, 11 mmol) was added over 10 minutes, and, after 30 minutes, the reaction mixture was allowed to warm to room temperature and stir overnight. The reaction mixture was cooled in an ice bath. The precipitate was filtered, washed with EA and dried over anhydrous Na₂SO₄. The product was dissolved in 10 ml of THF and used without further purification. UPLC-MS (method 1): Rt=1.43, m/z=433.24 [M+H]+.

Step 2. To a solution of tri(ethylene glycol) bis(N-hydroxysuccinimide) (0.532 g, 1.2 mmol) in THF (16 mL) was added 2-aminoethyl-mono-amide-DOTA-tris(t-Bu ester) (311.4 mg, 0.448 mmol) in several steps, and the solution was stirred at room temperature until the conversion reached ˜80%. Then, the reaction mixture was neutralized with 16 mL of aqueous TFA and purified by HPLC, yielding compound 010 (tri-tert-butyl 2,2′,2″-(10-(1-((2,5-dioxopyrrolidin-1-yl)oxy)-1,12,17-trioxo-2,5,8,11-tetraoxa-13,16-diazaoctadecan-18-yl)-1,4,7,10-tetraazacyclododecane-1,4,7-triyl)triacetate) (71 mg, 76 μmol, UV purity 91%, 15% yield). UPLC-MS (method 2): Rt=2.611, m/z=932.5 [M+H]+.

### Preparation of dihydroasparagusic acid-(Vedotin)₂ (Compound 011)

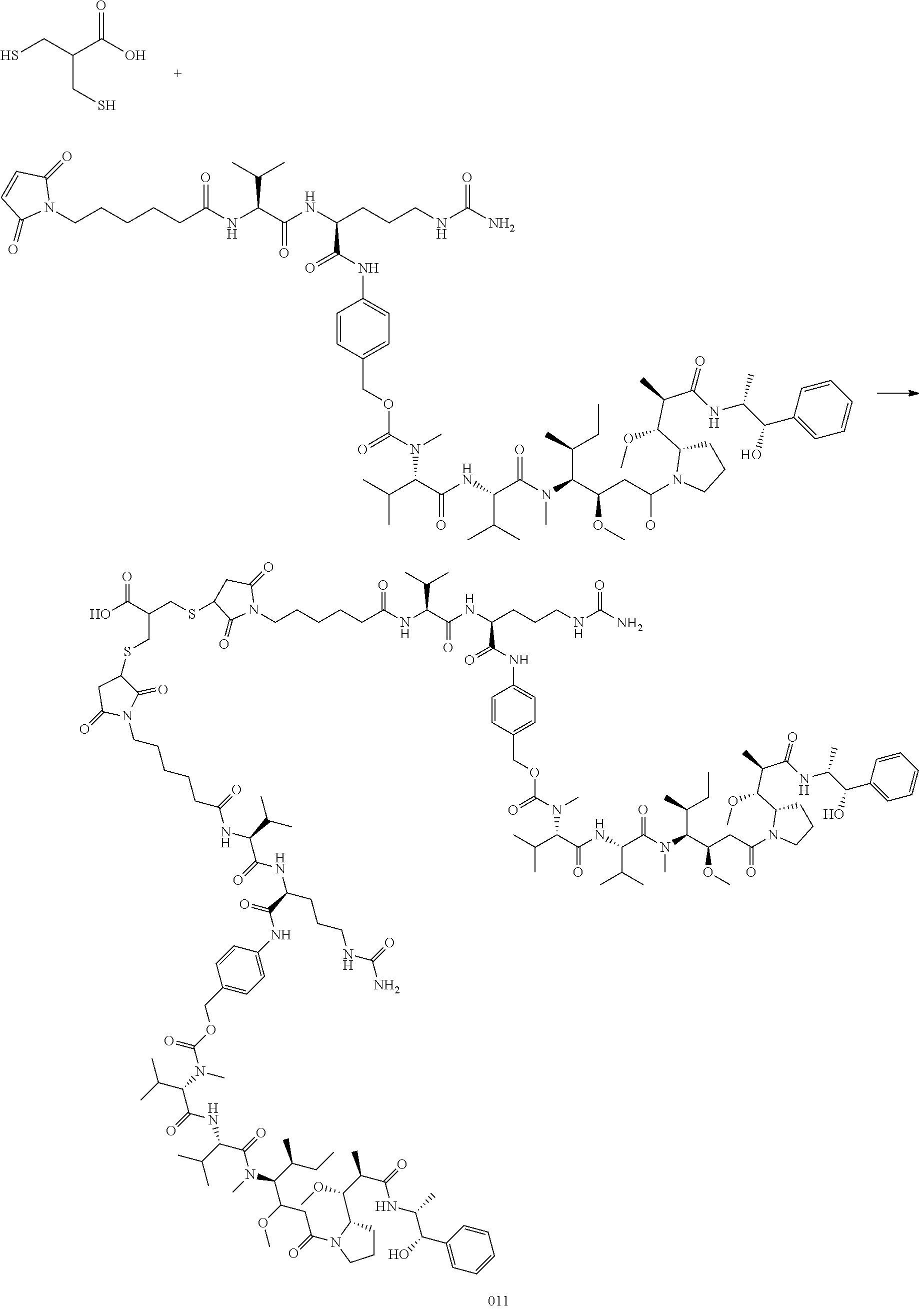

Dihydroasparagusic acid (0.5 mg, 3.0 μmol, 1.0 eq.) in solution in DMF (0.2 mL) followed by DIEA (3 μL, 17.7 μmol, 6.0 eq.) were added to a solution of Vedotin (7.8 mg, 6.0 μmol, 2.0 eq.) in DMF (0.2 mL) at rt. After stirring at rt for 20 min, TFA (3 μL) was added. Purification on 018 (12 g, 20 to 80% of ACN+0.1% TFA in water+0.1% TFA over 10 CV) afforded compound 011 (Dihydroasparagusic acid-(Vedotin)₂) (5.1 mg, 1.8 μmol, UV purity, 96%, 59% yield) as a white powder after freeze-drying. UPLC-MS (method 4): Rt=3.71 min m/z=1393 [M+2H]²⁺, 1414 [M-2H+FA]²⁻.

### Preparation of Compound 012

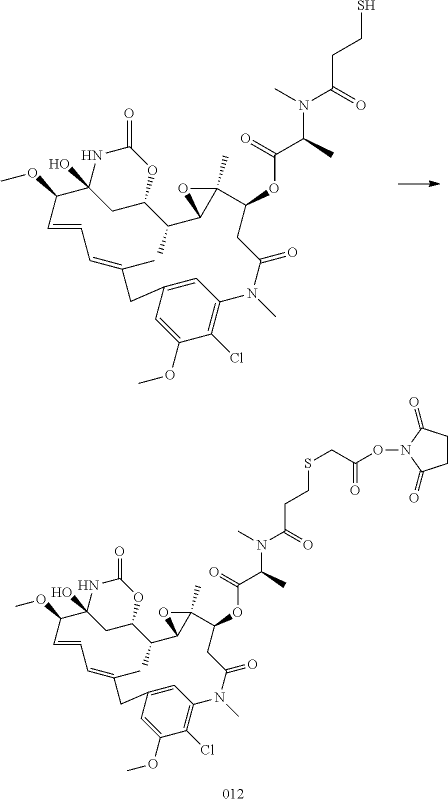 [SMILES: COc1cc2cc(c1Cl)N(C)C(=O)C[C@H](OC(=O)[C@H](C)N(C)C(=O)CCS)[C@]1(C)O[C@H]1[C@H](C)[C@@H]1C[C@@](O)(NC(=O)O1)[C@H](OC)/C=C/C=C(\C)C2 ; COc1cc2cc(c1Cl)N(C)C(=O)C[C@H](OC(=O)[C@H](C)N(C)C(=O)CCSCC(=O)ON1C(=O)CCC1=O)[C@]1(C)O[C@H]1[C@H](C)[C@@H]1C[C@@](O)(NC(=O)O1)[C@H](OC)/C=C/C=C(\C)C2]

DIEA (85 μL, 0.49 mmol, 6.0 eq.) was added to a solution of bromoacetic acid (13.7 mg, 0.14 mmol, 1.7 eq.) and DM1 (60.0 mg, 81.3 μmol, 1.0 eq.) in DMF (0.6 mL) at rt. After stirring at rt for 1 h, more bromoacetic acid (2.0 mg, 20 μmol, 0.25 eq.) was added to the reaction mixture. After stirring at rt for 10 min, a mixture of HATU·HPFn (34.0 mg, 89.4 μmol, 1.1 eq.) and 1-hydroxypyrrolidine-2,5-dione (10.3 mg, 89.4 μmol, 1.1 eq.) was added. After 30 min stirring at rt, purification by reversed phase automated flash chromatography 018 biotage column (30 g, 20 to 80% ACN+0.1% TFA in water+0.1% TFA) afforded compound 012 (36.1 mg, 40.4 μmol, UV purity 99%, 50% yield) as a white powder after freeze-drying. UPLC-MS (method 4): Rt=1.71 min, m/z=893 [M+H]*, 891 [M−H]⁻.

### Preparation of Compound 013

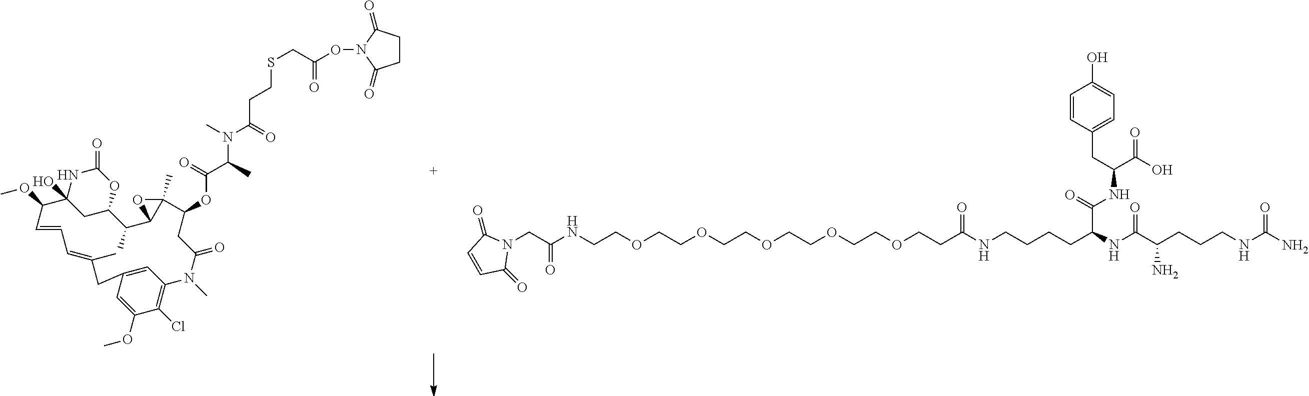 [SMILES: COc1cc2cc(c1Cl)N(C)C(=O)C[C@H](OC(=O)[C@H](C)N(C)C(=O)CCSCC(=O)ON1C(=O)CCC1=O)[C@]1(C)O[C@H]1[C@H](C)[C@@H]1C[C@@](O)(NC(=O)O1)[C@H](OC)/C=C/C=C(\C)C2 ; NC(=O)NCCC[C@H](N)C(=O)N[C@@H](CCCCNC(=O)CCOCCOCCOCCOCCOCCNC(=O)CN1C(=O)C=CC1=O)C(=O)N[C@@H](Cc1ccc(O)cc1)C(=O)O]

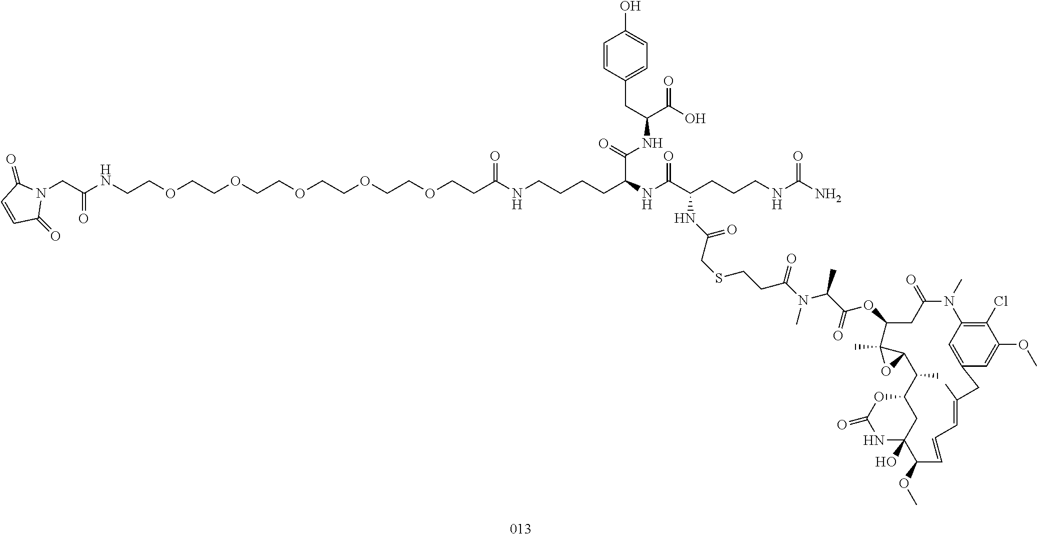 [SMILES: COc1cc2cc(c1Cl)N(C)C(=O)C[C@H](OC(=O)[C@H](C)N(C)C(=O)CCSCC(=O)N[C@@H](CCCNC(N)=O)C(=O)N[C@@H](CCCCNC(=O)CCOCCOCCOCCOCCOCCNC(=O)CN1C(=O)C=CC1=O)C(=O)N[C@@H](Cc1ccc(O)cc1)C(=O)O)[C@]1(C)O[C@H]1[C@H](C)[C@@H]1C[C@@](O)(NC(=O)O1)[C@H](OC)/C=C/C=C(\C)C2]

H-Cit-Lys(PEGs-ma)-Tyr-OH was purchased from Ambiopharm and prepared according to the following general procedure:

Peptide synthesis was performed on 2-CTC resin according to the general Fmoc/tBu strategy of solid phase peptide synthesis, with carboxyl group activation carried out by diisopropyl carbodiimide/HOBT. Sequentially, each amino acid was coupled as an active ester to the peptide chain, starting with the C-terminal amino acid. The final amino acid in the sequence was coupled with an N-terminally protected Boc group. The Lys derivative was incorporated with the side chain amino group protected by ivDde which was removed with 2% hydrazine in DMF. Following ivDde removal, the side chain was derivatized with maleimido-PEGs-OH using the activated ester. Subsequently, the peptide was treated with a TFA-based acidolytic cocktail which resulted in its cleavage from the resin and deprotection of the side chain groups. The peptide was then purified by liquid chromatography (RP-HPLC). The purified peptide TFA salt was lyophilized and obtained as a white to off-white powder.

DIEA (11.5 μL, 66.0 μmol, 2.0 eq.) was added to a mixture of H-Cit-Lys(PEG₅-ma)-Tyr-OH peptide (29.6 mg, 33.0 μmol, 1.0 eq.) and compound 012 (29.5 mg, 33.0 μmol, 1.0 eq.) in DMF (0.6 mL) at rt. After stirring at rt for 80 min, a drop of TFA was added. Purification by reversed phase automated flash chromatography on a C18 biotage column (30 g, 20 to 80% of ACN+0.1% TFA in water+0.1% TFA over 12CV) afforded compound 013 (41.4 mg, 23.8 μmol, UV purity 96%, 72% yield) as a white powder after freeze-drying. UPLC-MS (method 4): Rt=2.45 min, m/z=1673 [M+H]⁺, 1671 [M−H]⁻.

### Preparation of Compound 016

H-Cit-ε-azido-Nle-Tyr(tBu)-OtBu·HCl was purchased from Bachem and prepared according to the following generic procedure. Fmoc-Cit-Osu was coupled with H-ε-azido-Nle-OH to make the dipeptide Fmoc-Cit-ε-azido-Nle-OH, which was next reacted with H-Tyr(tBu)-OtBu to afford the tripeptide Fmoc-Cit-ε-azido-Nle-Tyr(tBu)-OtBu. The tripeptide was treated with piperidine to afford the crude tripeptide. Finally, the peptide was purified, and salt exchanged to afford final peptide H-Cit-ε-azido-Nle-Tyr(tBu)-OtBu. ma-PEG₅-[Lys(POC)-PEG₅-Arg]₂-Gly-CONH₂ was sourced from Ambiopharm and prepared according to the same procedure as H-Cit-Lys(PEG₅-ma)-Tyr-OH.

Step 1. TFA (1 mL) was added to H-Cit-ε-azido-Nle-Tyr(tBu)-OtBu·HCl (20 mg, 31.2 μmol, 1.0 eq.) at rt. After stirring at rt for 2 h, the reaction mixture was concentrated in vacuo. H₂O (2 mL) and ACN (2 mL) were added and the mixture was freeze-dried to afford H-Cit-B-azido-Nle-Tyr-OH (compound 014) (21.9 mg, 31.1 μmol, quantitative yield) as a white powder. UPLC-MS (method 3): Rt=0.73 min, m/z=493 [M+H]⁺, 491 [M−H]⁻.

Step 2. DIEA (19.5 μL, 0.11 mmol, 2.0 eq.) was added to a mixture of H-Cit-e-azido-Nle-Tyr-OH (compound 014) (39.4 mg, 56.0 μmol, 1.0 eq.) and compound 012 (50 mg, 56.0 μmol, 1.0 eq.) in DMF (2 mL) at rt. After 20 min stirring at rt, a drop of TFA was added. Purification on C18 (30 g, 20 to 80% of ACN+0.1% TFA in water+0.1% TFA over 8 CV) afforded compound 015 (45.8 mg, 31.4 μmol, UV purity 87%, 56% yield) as a white powder after freeze-drying. UPLC-MS (method 4): Rt=2.66 min, m/z=1270 [M+H]⁺, 1268 [M−H]⁻.

Step 3. A Schlenk tube previously charged with maleimide-PEG₅-(Lys(Poc)-PEG₅-Arg)₂-Gly-CONH₂*2TFA (43.4 mg, 21.2 μmol, 1.0 eq.), copper(I) iodide (4.0 mg, 21.2 μmol, 1.0 eq.), THPTA (9.2 mg, 21.2 μmol, 1.0 eq.) and compound 015 (53.9 mg, 42.4 μmol, 2.0 eq.) was purged three times with N₂. Degassed DMF (2.1 mL) and degassed DIEA (29.6 μL, 0.17 mmol, 8.0 eq.) were added to the reaction mixture. After stirring at rt for 1 h, 0.5 mL of a 0.5% TFA solution in ACN/H₂O (1:1) was added. Purification on C18 (30 g, 20 to 60% of ACN+0.1% TFA in water+0.1% TFA over 12CV) afforded compound 016 (47.7 mg, 9.3 μmol, UV purity 85%, 44% yield) as a white powder after freeze-drying. UPLC-MS (method 4): Rt=2.55 min, m/z=1454 [M+2H]²⁺, 1452 [M-2H]²⁻.

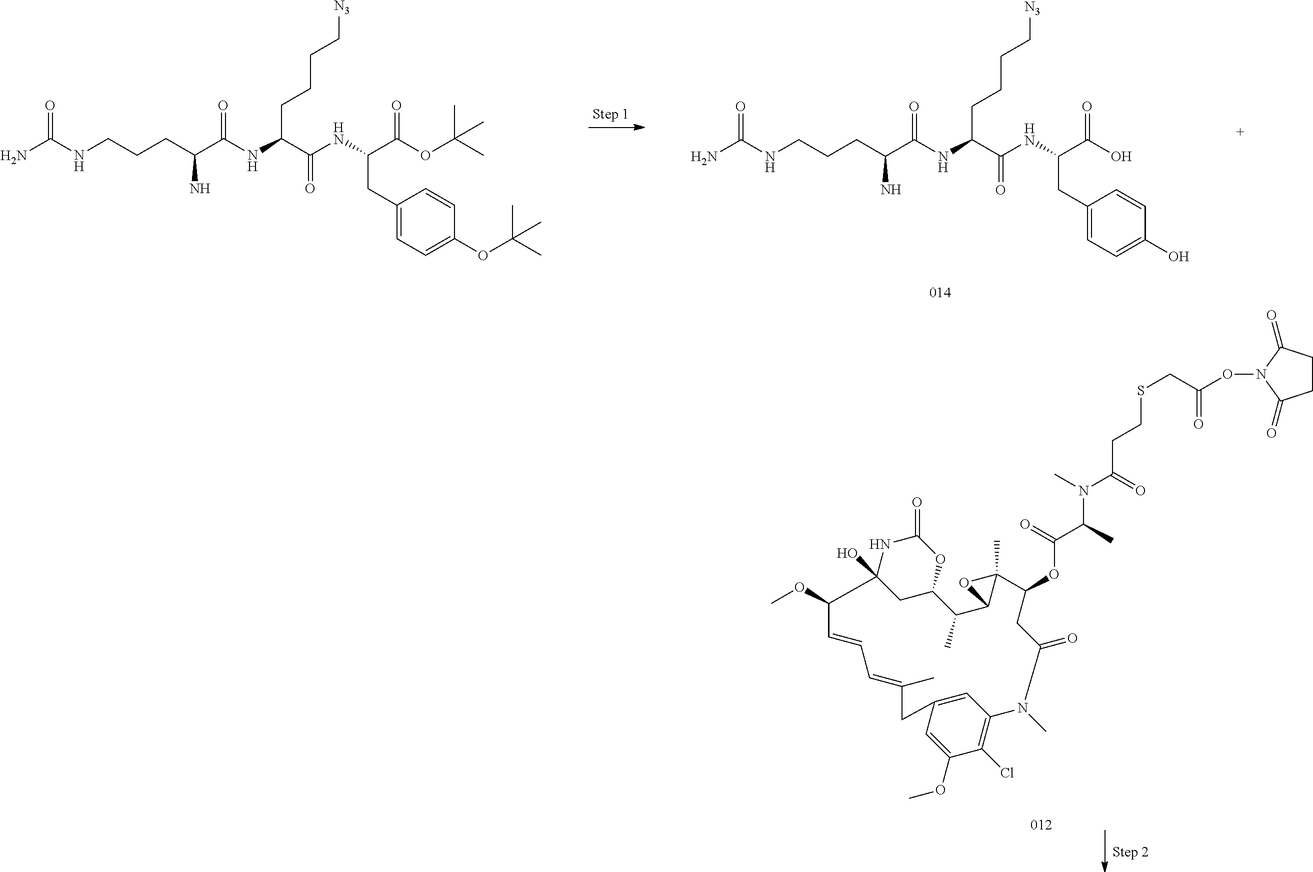 [SMILES: CC(C)(C)OC(=O)[C@H](Cc1ccc(OC(C)(C)C)cc1)NC(=O)[C@H](CCCCN=[N+]=[N-])NC(=O)[C@@H](N)CCCNC(N)=O ; [N-]=[N+]=NCCCC[C@H](NC(=O)[C@@H](N)CCCNC(N)=O)C(=O)N[C@@H](Cc1ccc(O)cc1)C(=O)O ; COc1cc2cc(c1Cl)N(C)C(=O)C[C@H](OC(=O)[C@H](C)N(C)C(=O)CCSCC(=O)ON1C(=O)CCC1=O)[C@]1(C)O[C@H]1[C@H](C)[C@@H]1C[C@@](O)(NC(=O)O1)[C@H](OC)/C=C/C=C(\C)C2]

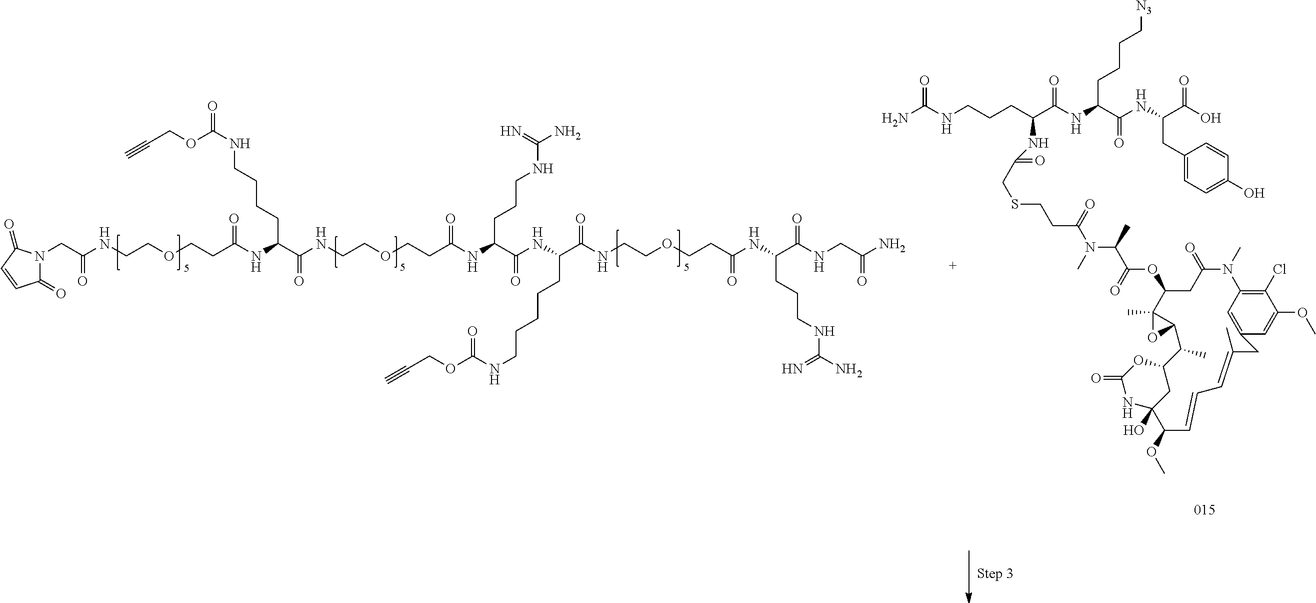

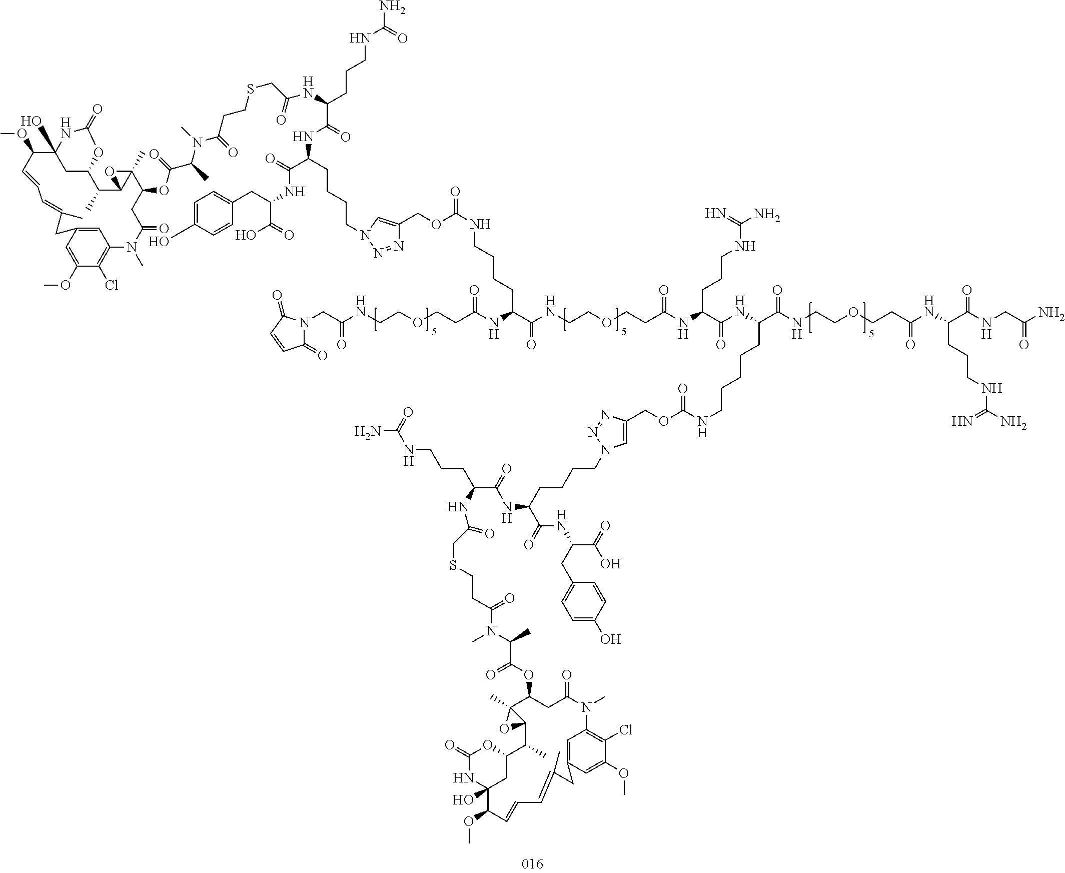

### Preparation of Boc-HN-PEG₃-carbonate-COOH (Compound 017)

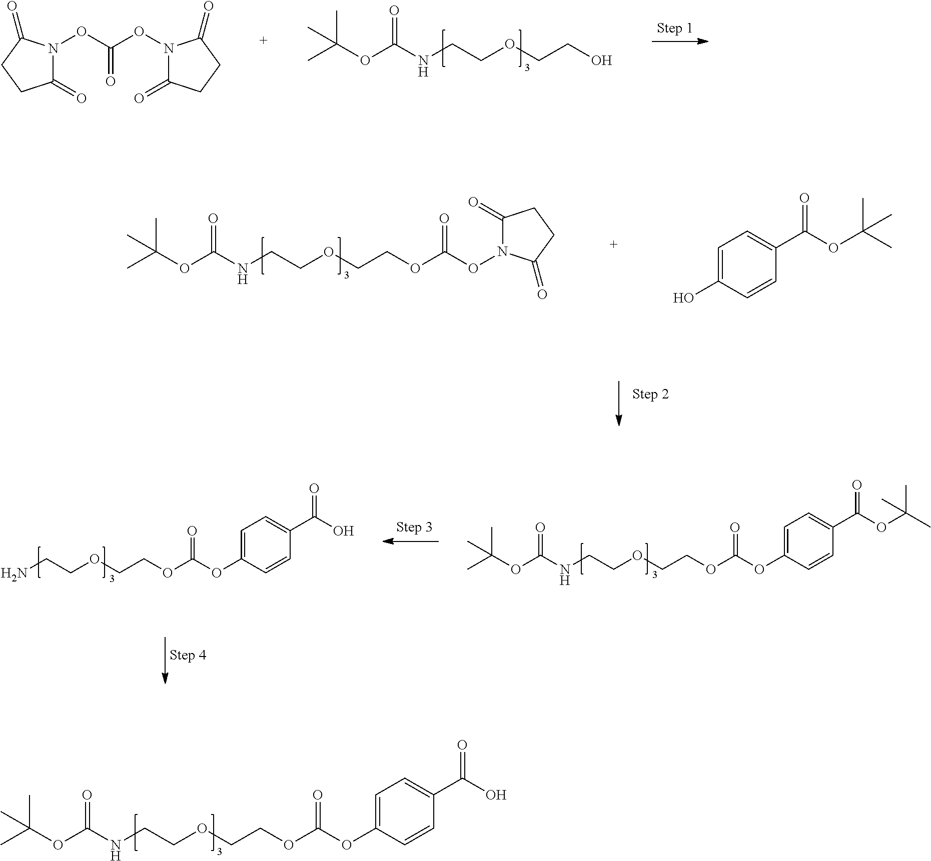

Step 1. DIEA (0.95 mL, 5.45 mmol, 4.0 eq.) was added to a mixture of bis(2,5-dioxopyrrolidin-1-yl) carbonate (1.47 g, 5.45 mmol, 4.0 eq.) and tert-butyl (2-(2-(2-(2-hydroxyethoxy)ethoxy)ethoxy)ethyl)carbonateamate (400.0 mg, 1.36 mmol, 1.0 eq.) in ACN (6 mL) at rt. After stirring at rt for 3 h, TFA was added until acidic pH was reached. Purification by preparative HPLC (10 to 50% of ACN+0.1% TFA in water+0.1% TFA) afforded 2-[2-[2-[2-(tert-butoxycarbonateonylamino)ethoxy]ethoxy]ethoxy]ethyl (2,5-dioxopyrrolidin-1-yl) carbonate (206.0 mg, 474.2 μmol, UV purity 100%, 35% yield) as a colourless oil after freeze-drying. UPLC-MS (method 4): Rt=1.98 min, m/z=336 [M-Boc+H]*, 479 [M+FA-H]⁻; 1H NMR (CDCl₃, 400 MHz) δ 5.03 (br s, 1H), 4.51-4.43 (m, 2H), 3.83-3.75 (m, 2H), 3.69-3.61 (m, 10H), 3.58-3.51 (m, 2H), 3.35-3.27 (m, 2H), 2.83 (s, 4H), 1.44 (s, 9H); 13C NMR (CDCl₃, 400 MHz) δ 171.4, 168.7, 156.2, 151.8, 71.1, 70.75, 70.70, 70.4, 70.3, 68.5, 53.6, 28.6, 25.6.

Step 2. 2-[2-[2-[2-(tert-butoxycarbonateonylamino)ethoxy]ethoxy]ethoxy]ethyl (2,5-dioxopyrrolidin-1-yl) carbonate (190.0 mg, 437.3 μmol, 1.0 eq.), tert-butyl 4-hydroxybenzoate (88.5 mg, 437.3 μmol, 1.0 eq.) and 4-(Dimethylamino)pyridine (107.9 mg, 874.7 μmol, 2.0 eq.) were stirred in DCM (2 mL) at rt for 1.5 h then the reaction mixture was concentrated in vacuo. Purification on silica (25 g, 5 to 100% of EtOAc in heptane over 12 CV) afforded tert-butyl 4-[2-[2-[2-[2-(tert-butoxycarbonateonylamino)ethoxy]ethoxy]ethoxy]ethoxycarbonateonyloxy]benzoate (161.9 mg, 31.5 μmol, UV purity 100%, 72% yield) as a colourless oil after concentration in vacuo. UPLC-MS (method 4): Rt=3.33 min, m/z=359 [M-Boc-tBu]*, 415 [M-Boc]+, 558 [M+FA-H]⁻; 1H NMR (CDCl₃, 400 MHz) δ 8.04-7.98 (m, 2H), 7.25-7.20 (m, 2H), 4.44-4.39 (m, 2H), 3.84-3.78 (m, 2H), 3.73-3.67 (m, 4H), 3.67-3.60 (m, 4H), 3.54 (t, J=5.2 Hz, 2H), 3.31 (t, J=5.1 Hz, 2H), 1.58 (s, 9H), 1.43 (s, 9H); 13C NMR (CDCl₃, 400 MHz) δ 165.0, 156.2, 154.3, 153.2, 131.2, 129.9, 120.9, 81.4, 70.9, 70.8, 70.4, 68.9, 68.0, 28.6, 28.3.

Step 3. Trifluoroacetic acid (0.7 mL) was added to a solution of tert-butyl 4-[2-[2-[2-[2-(tert-butoxycarbonateonylamino)ethoxy]ethoxy]ethoxy]ethoxycarbonateonyloxy]benzoate (161.9 mg, 315.2 μmol, 1.0 eq.) in DCM (0.7 mL) at rt. After stirring at rt for 30 min, the reaction mixture was concentrated in vacuo affording 4-[2-[2-[2-(2-aminoethoxy)ethoxy]ethoxy]ethoxycarbonateonyloxy]benzoic acid (125 mg, 314.8 μmol, UV purity 90%, 99.9% yield) as a colourless oil. The product was used in the next step without purification. UPLC-MS (method 4): Rt=1.08 min, m/z=358 [M+H]⁺, 357 [M−H]⁻.

Step 4. Triethylamine (0.18 mL, 1.26 mmol, 4.0 eq.) was added to a mixture of 4-[2-[2-[2-(2-aminoethoxy)ethoxy]ethoxy]ethoxycarbonateonyloxy]benzoic acid (125.17 mg, 0.32 mmol, 1.0 eq.) and di-tert-butyl dicarbonate (139.00 mg, 0.63 mmol, 2.0 eq.) in DCM (0.7 mL) and DMF (0.7 mL) at rt. After stirring at rt for 20 min, TFA was added until acidic pH was reached. Purification on silica (20-100% EA in heptane then neat DCM) afforded the desired product along with DMF. Purification by preparative HPLC (20 to 50% of ACN+0.1% TFA in water+0.1% TFA) afforded BocHN-PEG₃-carbonate-COOH (21.6 mg, 47.2 μmol, UV purity 100%, 15% yield) as a colourless oil after freeze-drying. UPLC-MS (method 4): Rt=2.26 min, m/z=358 [M-Boc+H]⁺, 458 [M−H]⁻.

### Preparation of acid-carbonate-N₃ (Compound 018)

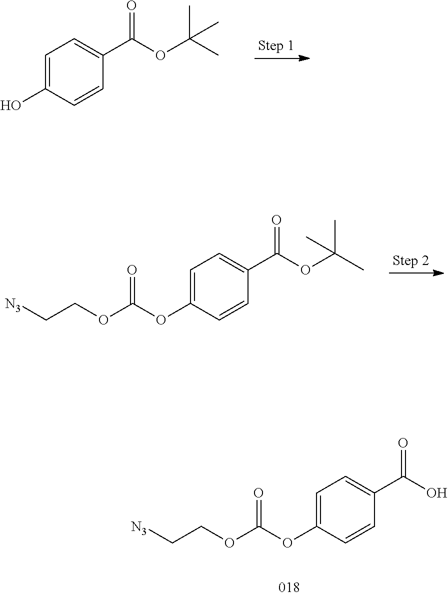 [SMILES: CC(C)(C)OC(=O)c1ccc(O)cc1 ; CC(C)(C)OC(=O)c1ccc(OC(=O)OCCN=[N+]=[N-])cc1 ; [N-]=[N+]=NCCOC(=O)Oc1ccc(C(=O)O)cc1]

Step 1. DIEA (0.3 mL, 1.7 mmol, 2.5 eq.) was added to a solution of bis(2,5-dioxopyrrolidin-1-yl) carbonate (373.20 mg, 1.4 mmol, 2.0 eq.) and 2-azidoethanol (60.30 mg, 692.5 μmol, 1.0 eq.) in anhydrous ACN (7 mL). After stirring at rt for 4.5 h, DMAP (169.20 mg, 1.4 mmol, 2.0 eq.) and tert-butyl 4-hydroxybenzoate (403.49 mg, 2.1 mmol, 3.0 eq.) were added to the reaction mixture. After stirring at rt for 25 h, TFA was added until acidic pH was reached. Purification on C18 (30g, 20 to 98% of ACN+0.1% TFA in water+0.1% TFA over 12 CV) afforded tert-butyl 4-(2-azidoethoxycarbonateonyloxy)benzoate (138.00 mg, 426.6 μmol, 95% UV purity, 62% yield) as a colorless oil after freeze-drying. ¹H NMR (CDCl₃, 400 MHz) δ 7.96 (d, J=8.9 Hz, 2H), 7.17 (d, J=8.9 Hz, 2H), 4.34 (t, J=5.1 Hz, 2H), 3.54 (3.54 Hz, J=5.1 Hz, 2H), 1.52 Hz (s, 9H); ¹³C NMR (CDCl₃, 100 MHz) δ 164.8, 154.0, 152.8, 131.1, 130.0, 120.7, 81.4, 66.9, 49.5, 28.2.

Step 2. Trifluoroacetic acid (1.5 mL) was added to a mixture of tert-butyl 4-(((2-azidoethoxy)carbonateonyl)oxy)benzoate (138.00 mg, 449.1 μmol, 1.0 eq.) in DCM (4 mL) at rt. The reaction mixture was stirred at rt for 1 h then concentrated in vacuo. Water (5 mL) and ACN (5 mL) were added and the mixture was freeze-dried to afford acid-carbonate-N₃ (71.10 mg, 283.0 μmol, 100% UV purity, 63% yield) as a white powder after freeze-drying. UPLC-MS (method 3): Rt=1.47 min, m/z=250 [M−H]—; NMR ¹H (DMSO, 400 MHz) δ 8.01 (d, J=8.7 Hz, 2H), 7.38 (d, J=8.7 Hz, 2H), 4.39 (t, J=8.7 Hz, 2H), 3.68 (t, J=8.7 Hz, 2H); ¹³C NMR (DMSO, 100 MHz) δ 166.9, 154.3, 152.8, 131.5, 129.2, 129.2, 121.9, 67.8, 49.5.

### Preparation of carbonate-PEG₅-Bn-NOTA (Compound 019)

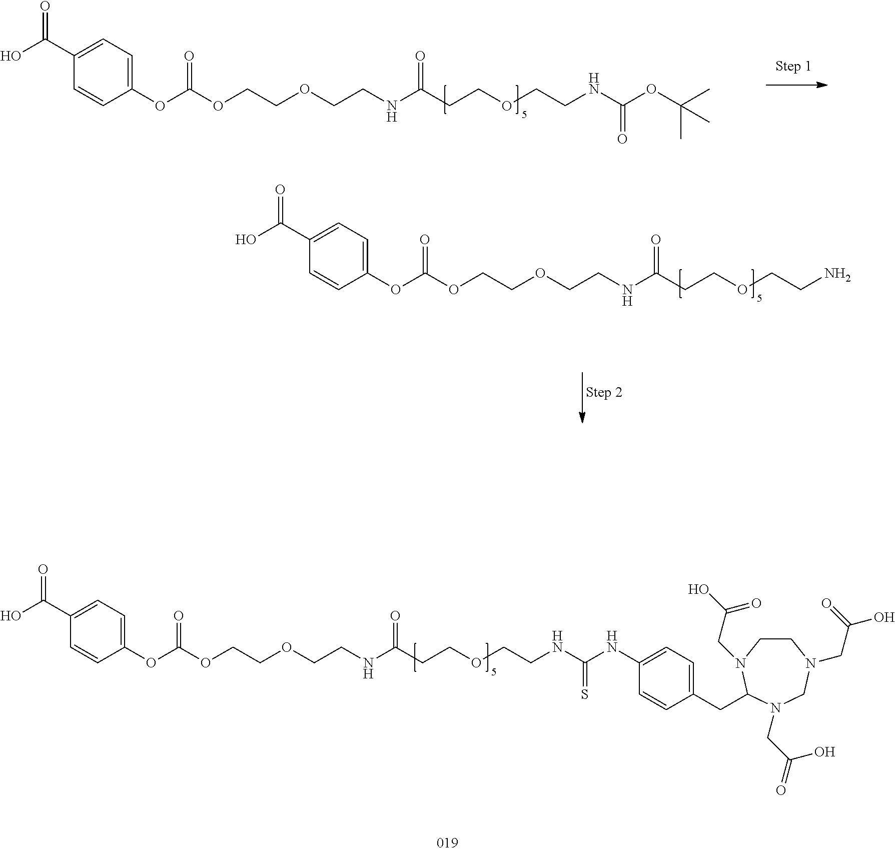

Step 1. Trifluoroacetic acid (1 mL) was added to a solution of HOOC-carbonate-PEG₁-CO-PEG₅-NH-Boc (11.00 mg, 20.0 μmol, 1.0 eq.) in DCM (2 mL) at rt. The reaction mixture was stirred at rt for 2 h then concentrated in vacuo. Water (7.5 mL) and ACN (7.5 mL) were added and the mixture was freeze-dried to afford HOOC-carbonate-PEG₁-CO-PEG₅-NH₂ (16.43 mg, 29.3 μmol, 100% UV purity, assumed quant.) as a colorless oil after freeze-drying. UPLC-MS (method 3): Rt=0.90 min, m/z=561 [M+H]*, 559 [M−H]⁻.

Step 2. DIEA (20 μL, 110 μmol, 3.9 eq.) was added to a mixture of p-SCN-Bn-NOTA (18.10 mg, 30.0 μmol, 1.1 eq.) and HOOC-carbonate-PEG₁-CO-PEG₅-NH₂ (16.43 mg, 29.3 μmol, 1.0 eq.) in DMF (1.5 mL). After stirring at rt for 4 h, TFA was added until acidic pH was reached. Purification by preparative HPLC (10 to 80% ACN+0.1% TFA in water+0.1% TFA) afforded HOOC-carbonate-PEG₁-CO-PEG₅-Bn-NOTA (14.30 mg, 14.1 μmol, 100% UV purity, 48% yield) as a white powder after freeze-drying. UPLC-MS (method 3): Rt=1.15 min, m/z=1011 [M+H]⁺, 506 [M+2H]2+, 1009 [M−H]⁻.

### Preparation of NHS-suc-NH—VC-PAB-MMAE Compound 020)

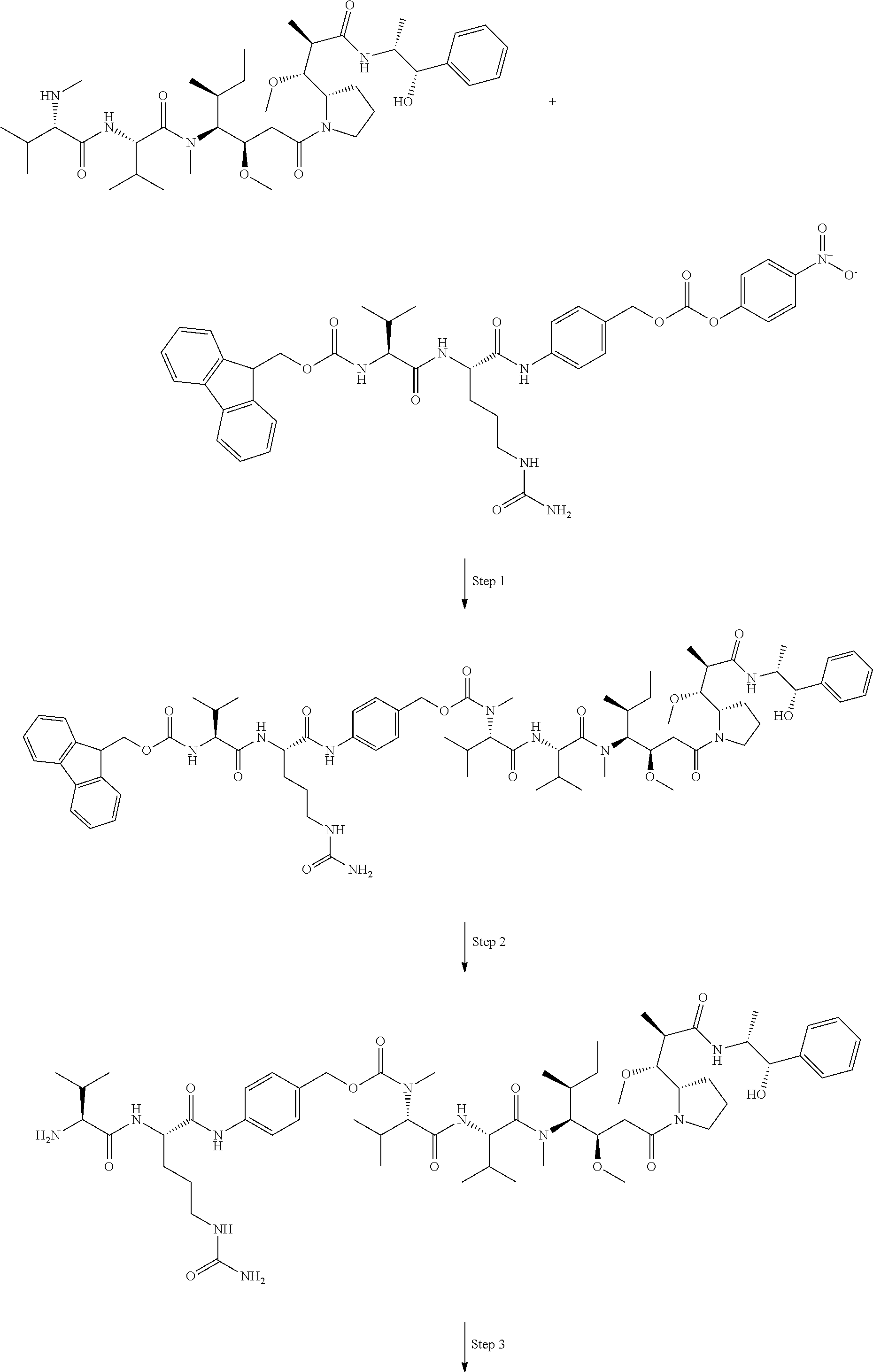

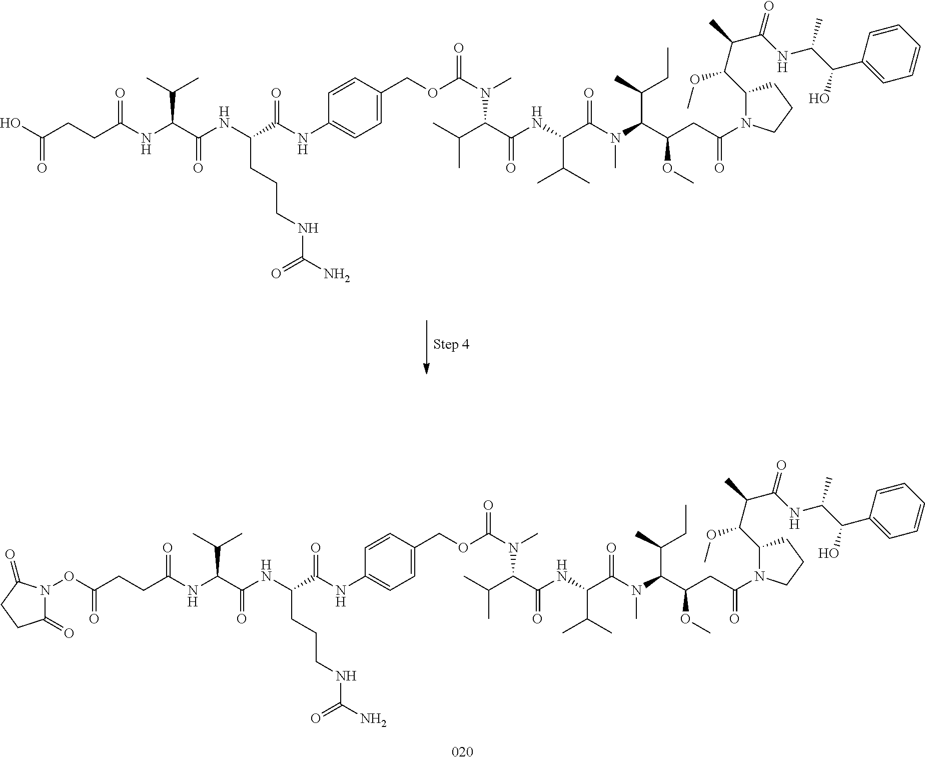 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCC(=O)O)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCC(=O)ON2C(=O)CCC2=O)C(C)C)cc1)C(C)C]

Step 1. HOBt (8.80 mg, 65.1 μmol, 0.5 eq. followed by pyridine mL, 3.7 mmol, 31.2 eq.) were added to a solution of MMAE (129.00 mg, 176.1 μmol, 1.5 eq.) and Fmoc-VC-PAB-OPNP (91.40 mg, 119.2 μmol, 1.0 eq.) in DMF (4 mL). After stirring at rt for 24 h, the mixture was poured into ethyl acetate (25 mL) and washed two times with water (20 mL). The aqueous phase was extracted two times with ethyl acetate (20 mL). The pooled organic phase was washed one time with citric acid 10% (10 mL), two times with water (15 mL) and one time with brine (15 mL). The organic layer was dried over MgSO₄, filtered, and concentrated under vacuo.

Purification by silica gel flash chromatography (25 g, 1% of MeOH in DCM over 7 CV followed by 10% of MeOH in DCM over 7 CV) afforded Fmoc-VC-PAB-MMAE (94.50 mg, 70.2 μmol, 100% UV purity, 59% yield) as a white solid after concentration in vacuo. UPLC-MS (method 5): Rt=1.97 min, 1345 [M+H]*, 1389 [M+FA-H]⁻, 1165 [M-Fmoc+CO₂—H]⁻.

Step 2. Piperidine (1.0 mL, 10.0 mmol, 107.1 eq.) was added to a solution of Fmoc-VC-PAB-MMAE (157.40 mg, 93.6 μmol, 1.0 eq.) in ACN (4 mL). After stirring at rt for 12 h, the reaction mixture was concentrated in vacuo. The resulting residue was diluted with DCM and then diethylether was added. The mixture was filtered, the cake was dissolved in DCM then concentrated in vacuo to afford NH₂—VC-PAB-MMAE (138.70 mg, 102.5 μmol, 70% UV purity, 92% yield) as a yellow oil. UPLC-MS (method 5): Rt=1.42 min, m/z=1123 [M+H]*, 1121 [M−H]⁻.

Step 3. dihydrofuran-2,5-dione (15.10 mg, 149.4 μmol, 1.2 eq.) followed by DIEA (0.2 mL, 1.2 mmol, 9.3 eq.) were added to a solution of NH₂—VC-PAB-MMAE (138.70 mg, 123.5 μmol, 1.0 eq.) in DMF (5 mL). After stirring at rt for 30 min, TFA was added until acidic pH was reached. Purification by preparative HPLC (20 to 80% of ACN+0.1% TFA in water+0.1% TFA) afforded suc-NH—VCVC-PAB-MMAE (100.00 mg, 81.7 μmol, 100% UV purity, 66% yield) as a white powder after freeze-drying. UPLC-MS (method 5): Rt=1.56 min, m/z=1223 [M+H]*, 1221 [M−H]⁻.

Step 4. DIEA (56.6 μL, 326.9 μmol, 4.0 eq.) was added to a mixture of suc-NH—VC-PAB-MMAE (100.00 mg, 81.7 μmol, 1.0 eq.) and HATU (31.08 mg, 81.7 μmol, 1.0 eq.) in DMF (4 mL) at rt. after stirring at rt for 5 min, 1-hydroxypyrrolidine-2,5-dione (9.41 mg, 81.7 μmol, 1.0 eq.) was added to the reaction mixture. After stirring at rt for 10 min, TFA was added until acidic pH was reached. Purification by preparative HPLC (20 to 80% of ACN+0.1% TFA in water+0.1% TFA) afforded NHS-suc-NH—VC-PAB-MMAE (93.70 mg, 66.7 μmol, 94% UV purity, 82% yield) as a white powder after freeze-drying. UPLC-MS (method 5): 1.60 min, m/z=1320 [M+H]*, 1364 [M+FA-H]⁻.

### Preparation of DBCO-NH-suc-NH—VC-PAB-MMAE (Compound 021)

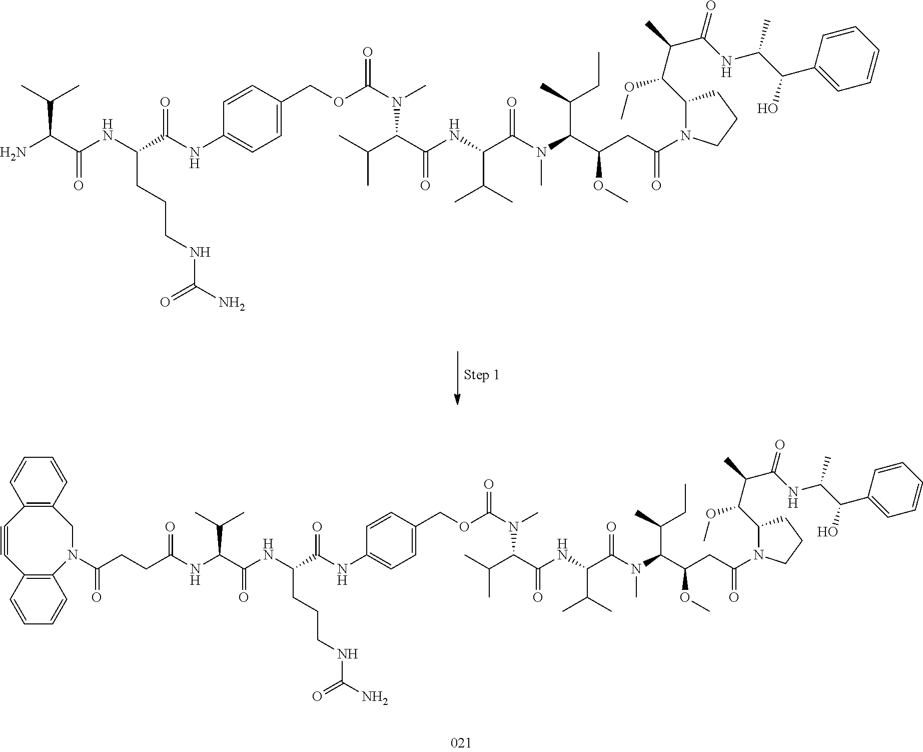 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCC(=O)N2Cc3ccccc3C#Cc3ccccc32)C(C)C)cc1)C(C)C]

H₂N-VC-PAB-MMAE (49.3 mg, 40.0 μmol, 1.0 eq.) and DBCO-NHS ester (18.00 mg, 40.0 μmol, 1.0 eq.) were diluted in DCM (1.2 mL). DIEA (30 μL, 180.0 μmol, 4.1 eq.) was added and the reaction mixture was stirred at rt for 2 h then concentrated under nitrogen flow and dried under vacuum for 30 min. Purification by preparative HPLC (30 to 100% of ACN+0.1% TFA in water+0.1% TFA) afforded DBCO-NH-suc-NH—VC-PAB-MMAE (68.00 mg, 40.9 μmol, 85% UV purity, 93% yield) after freeze-drying. UPLC-MS (method 3): Rt=2.18 min, m/z=1412 [M+H]*.

### Preparation of acid-PEG₁-VC-PAB-MMAE (Compound 022)

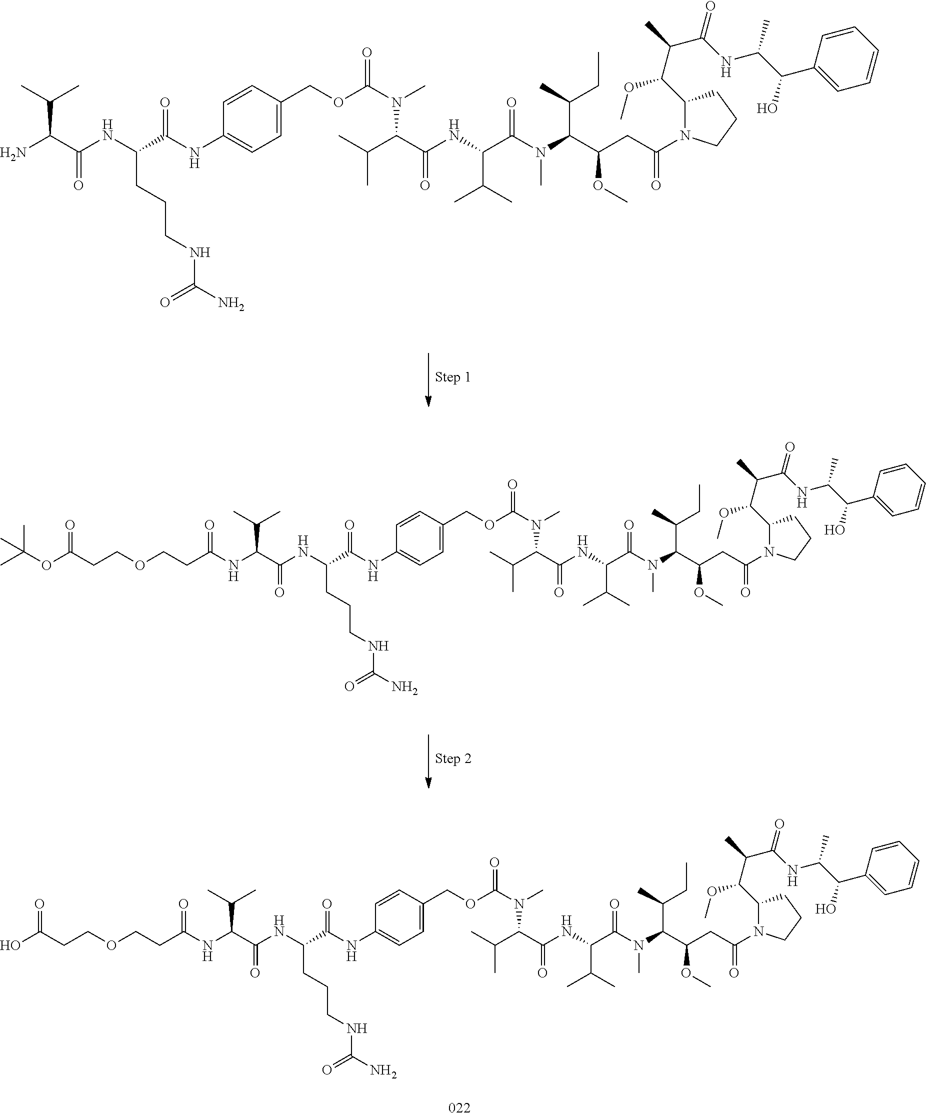

Step 1. H₂N-VC-PAB-MMAE (105.93 mg, 90.0 μmol, 1.05 eq.) and 3-(3-(tert-butoxy)-3-oxopropoxy)propanoic acid (20.0 mg, 90.0 μmol, 1.0 eq.) was diluted with DMF (1 mL). DIEA (60 μL, 360 μmol, 4.0 eq.) was added at rt followed by HATU (51.22 mg, 130 μmol, 1.5 eq.). After stirring at rt for 30 min, the reaction mixture was diluted with water. The resulting precipitate was collected by filtration. Purification by preparative HPLC (30 to 100% of ACN+0.1% TFA in water+0.1% TFA) afforded tBuOOC-PEG₁-VC-PAB-MMAE (87 mg, 64.1 μmol, 98% UV purity, 71% yield) as a white solid after freeze-drying. UPLC-MS (method 3): Rt=2.12 min m/z=1324 [M+H]*, 1322 [M−H].

Step 2. Trifluoroacetic acid (0.25 mL) was added to a solution of tBuOOC-PEG₁-VC-PAB-MMAE (78.00 mg, 60.0 μmol, 1.0 eq.) in DCM (0.5 mL). After stirring at rt for 35 min, the reaction mixture was concentrated in vacuo. Purification on 018 (30 g, 20 to 80% ACN+0.1% TFA in water+0.1% TFA) afforded acid-PEG₁-VC-PAB-MMAE (42.00 mg, 31.3 μmol, 95% UV purity, 53% yield) as a white solid after freeze-drying. UPLC-MS (method 3): Rt=1.85 min m/z=1268 [M+H]*, 1266 [M−H]⁻.

### Preparation of multi-(DM1)₂-PEG₁₅ (Compound 024)

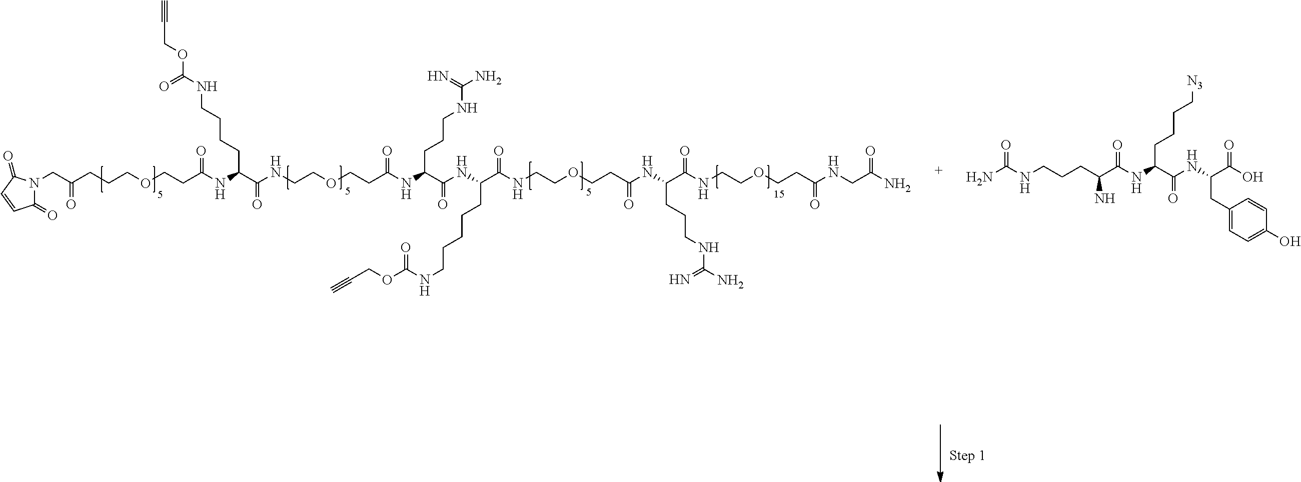

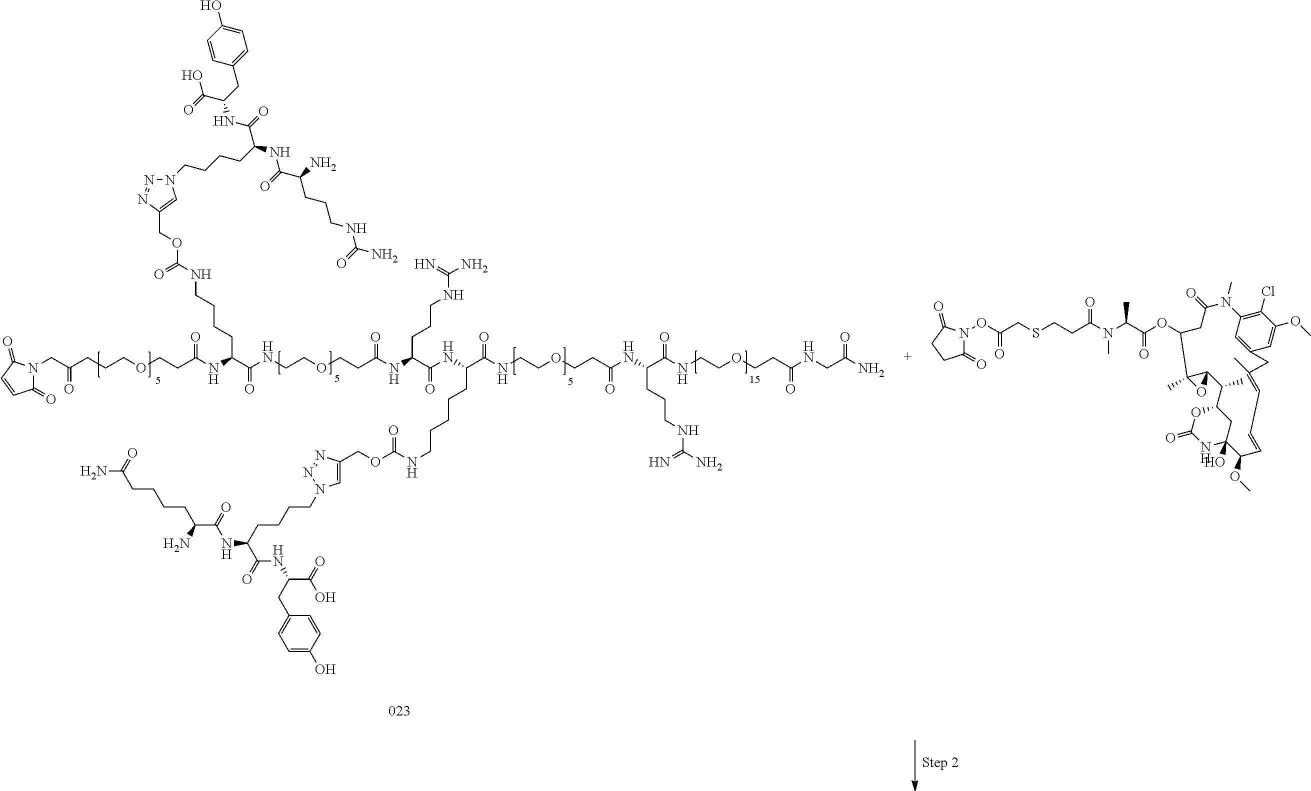

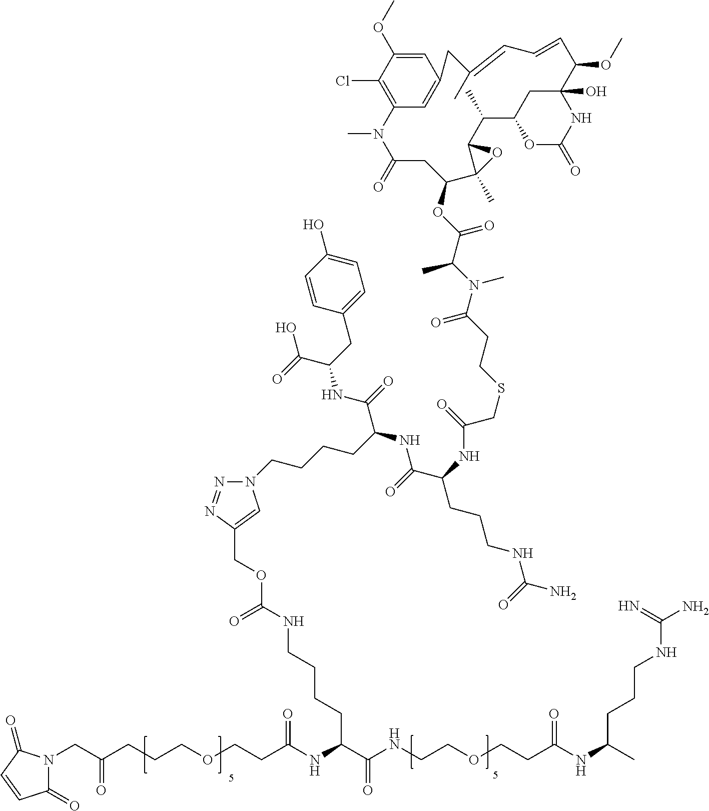

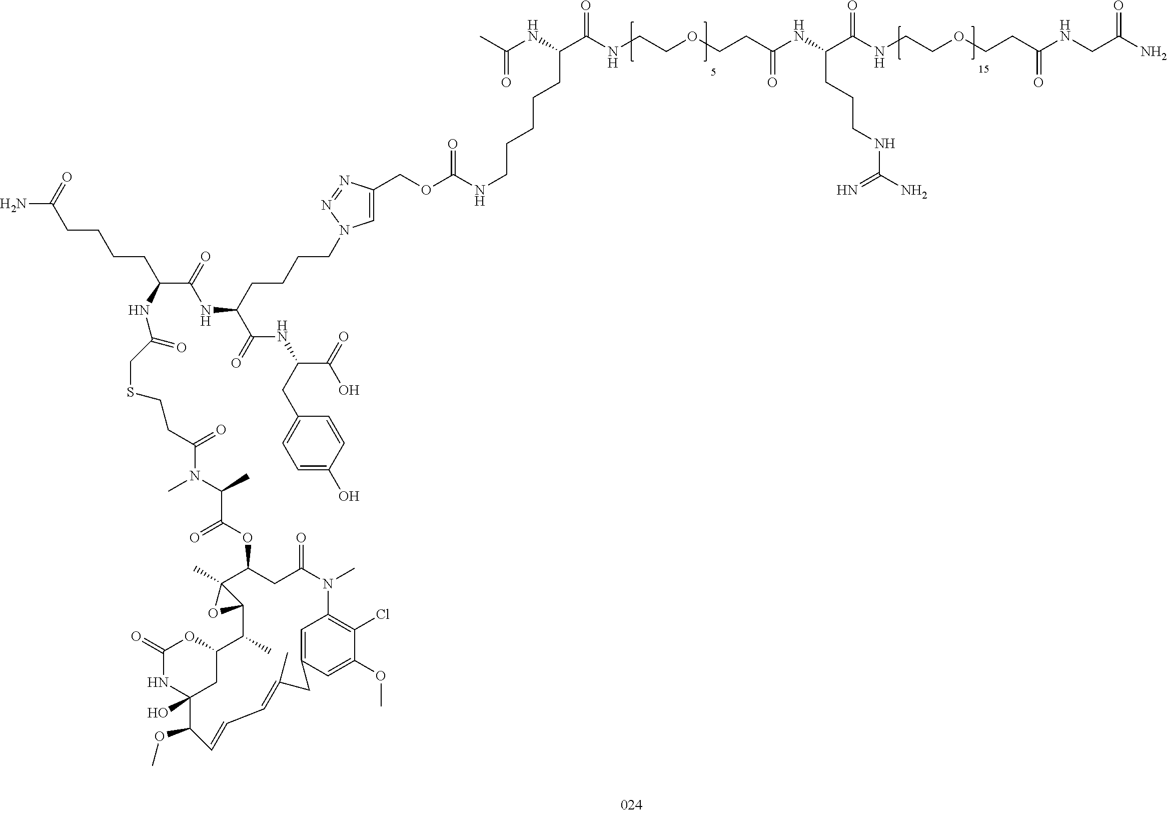

Step 1. A Schlenk tube previously charged with copper(I) iodide (5.98 mg, 31.4 μmol, 1.0 eq.), THPTA (13.63 mg, 31.4 μmol, 1.0 eq.), ma-PEG₅-(Lys(Poc)-PEG₅-Arg)-(Lys(Poc)-PEG₁₅-Arg)-Gly-CONH₂*2TFA (80.0 mg, 31.4 μmol, 1.0 eq.) and H-Cit-B-azido-Nle-Tyr-OH (54.22 mg, 62.7 μmol, 2.0 eq.) was purged three times with N₂ then degassed DMF (2 mL) was added to the reaction mixture. After stirring at rt for 16 h, a mixture of ACN/water/TFA (1:1:0.5%, 1 mL) was added. Purification by preparative HPLC (5 to 50% of ACN+0.1% TFA in water+0.1% TFA) afforded compound 023 (75.4 mg, 20.9 μmol, 98% UV purity, 67% yield) as a yellow powder after freeze-drying. UPLC-MS (method 4): Rt=1.44 min, m/z=1769 [M+2H]²⁺, 1767 [M-2H]²⁻.

Step 2. DIEA (7.7 μL, 44.4 μmol, 8.0 eq.) was added to a mixture of compound 023 (20.0 mg, 5.5 μmol, 1.0 eq.) and compound 012 (12.38 mg, 11.1 μmol, 2.0 eq.) in DMF (0.5 mL) at rt. After stirring at rt for 2 h, TFA was added until acidic pH was reached. Purification by preparative HPLC (5 to 100% of ACN+0.1% FA in water+0.1% FA) afforded compound 024 (8.50 mg, 1.7 μmol, 99% UV purity, 30% yield) as a white powder after freeze-drying. UPLC-MS (method 4): Rt=2.45 min, m/z=1698 [M+3H]³⁺, 1696 [M-3H]³⁻.

### Preparation of multi-(DM1)-K(PEG₂₄)-(DM1) (Compound 026)

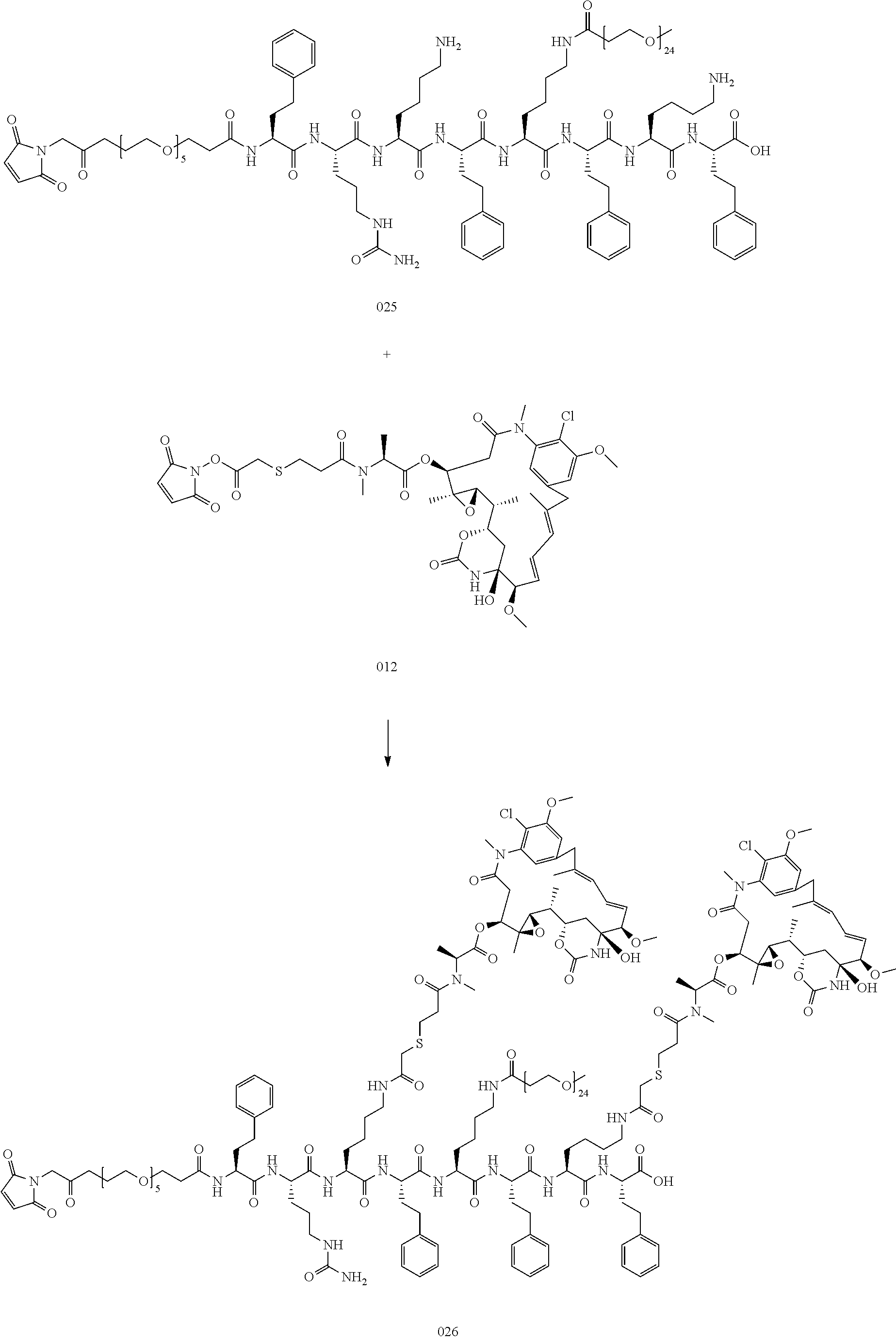

DIEA (1.3 μL, 7.20 μmol, 4.0 eq.) was added to a mixture of compound 025 (5.0 mg, 1.8 μmol, 1.0 eq.) and compound 012 (3.39 mg, 3.6 μmol, 2.0 eq.) in DMF (0.2 mL) at rt. After stirring at rt for 1.5 h, TFA was added until acidic pH was reached. Purification on C18 (12 g, 20 to 80% of ACN+0.1% TFA in water+0.1% TFA) afforded compound 026 (5.10 mg, 1.10 μmol, 97% UV purity, 63% yield) as a white powder after freeze-drying. UPLC-MS (method 4): Rt=3.59 min, m/z=1064 [M+4H]⁴*.

### Preparation of PNU-OPFP (Compound 027)

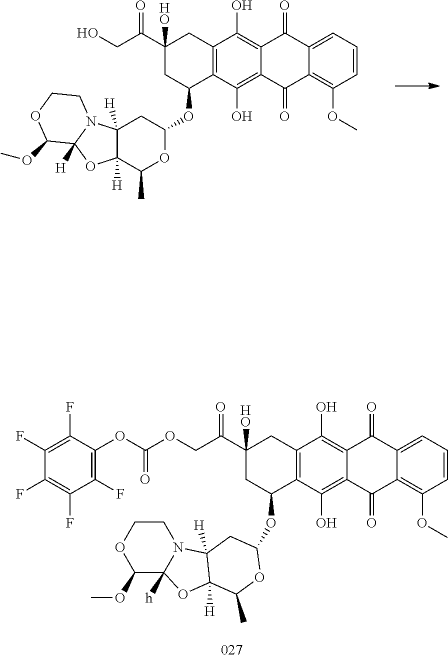

Five drops of TEA were added to a mixture of PNU-159682 (85.0 mg, 132.5 μmol, 1.0 eq.) and bis(perfluorophenyl) carbonate (242.2 mg, 59.6 μmol, 4.5 eq.) in a mixture of DCM (1.7 mL) and THF (1.7 mL) at rt. After stirring at rt for 1 h, four drops of TFA were added to the reaction mixture at rt. The reaction mixture was concentrated in vacuo (bath at 30° C.). The residue was dissolved in DMF (6 mL) then purified by preparative HPLC (40 to 100% of ACN-in water) to afford PNU-OPFP (compound 027) (87.2 mg, 102.4 μmol, 100% UV purity, 77% yield) as an orange powder after freeze-drying. UPLC-MS (method 3): Rt=2.39 min, m/z=852 [M+H]*, 850 [M−H]⁻.

### Example 5: Preparation of DOTA-carbonate-containing Reactive Conjugates

The Fc-binding vectors prepared in Example 1 were converted into reactive conjugates of formula (1) by coupling of compounds 006 or 007 to the amino group of the side chain of the respective Fc-binding vectors. Synthesis of compound D1K-DOTA is shown in a scheme below. The other DOTA-carbonate-peptide conjugates were prepared in a similar way. The structures of the DOTA-containing reactive conjugates prepared in Example 5 are shown in the table below.

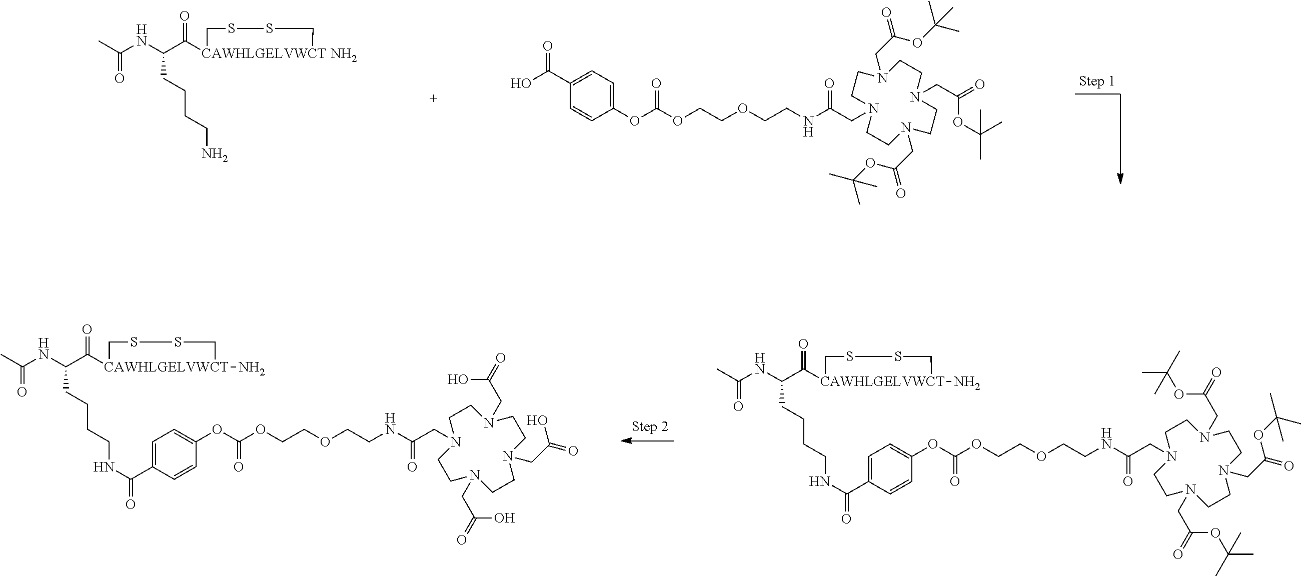

TABLE 5
Structures of DOTA-containing reactive conjugates

| Compound | Structure |
|---|---|
| D1K- carbonate- DOTA (D1K + 006) | 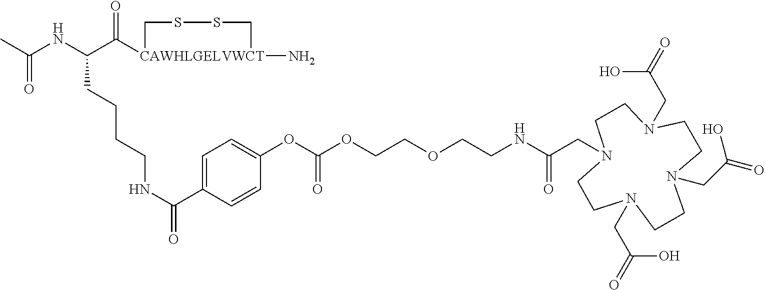 |
| A3K- carbonate- DOTA (A3K + 006) | 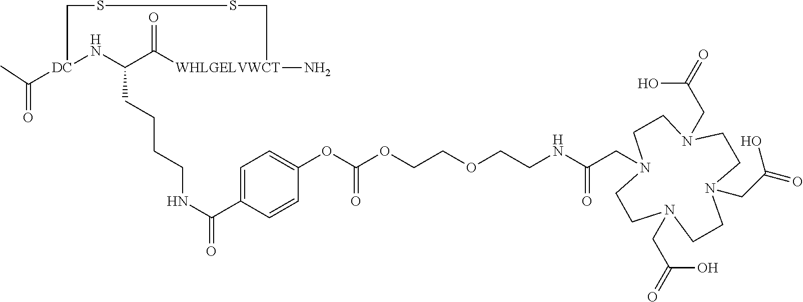 |
| W4K- carbonate- DOTA (W4K + 006) | 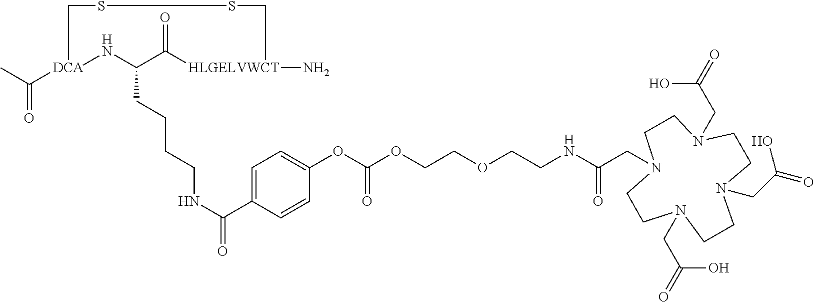 |
| H5K- carbonate- DOTA (H5K + 006) | 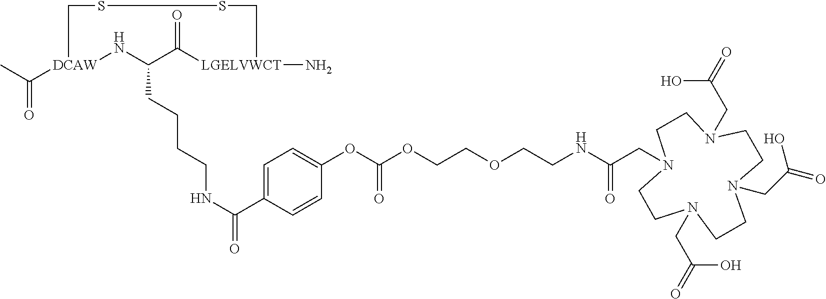 |
| L6K- carbonate- DOTA (L6K + 006) | 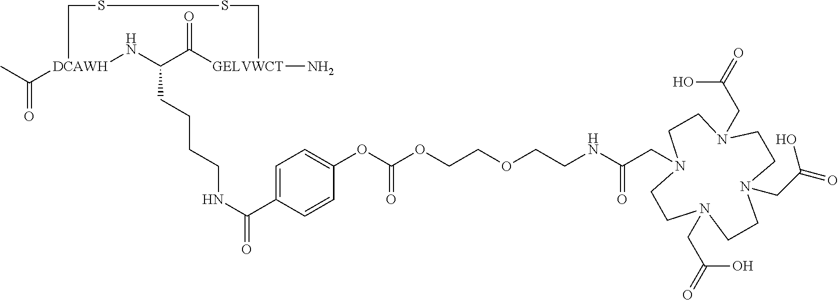 |
| G7K- carbonate- DOTA (G7K + 006) | 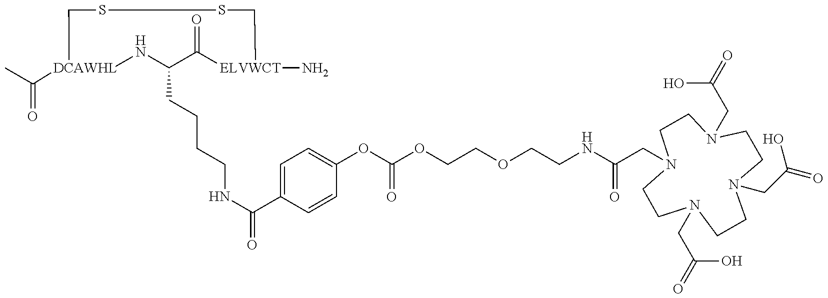 |
| E8K- carbonate- DOTA (E8K + 006) | 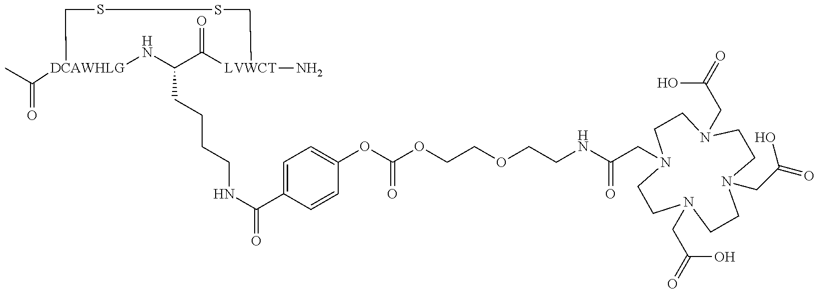 |
| L9K- carbonate- DOTA (L9K + 006) | 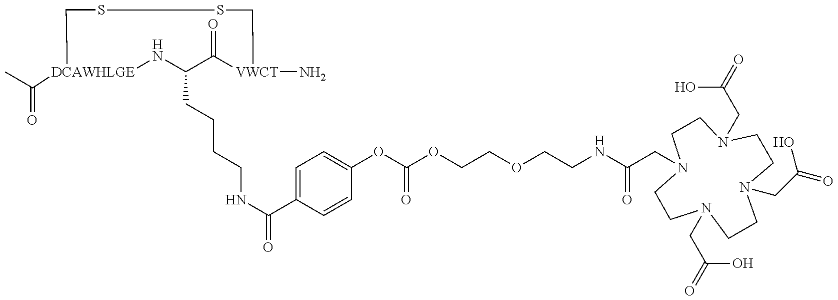 |
| V10K- carbonate- DOTA (V10K + 006) | 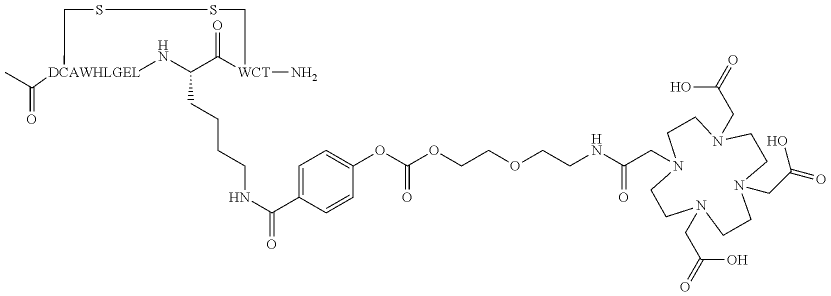 |
| T13K- carbonate- DOTA (T13K + 006) | 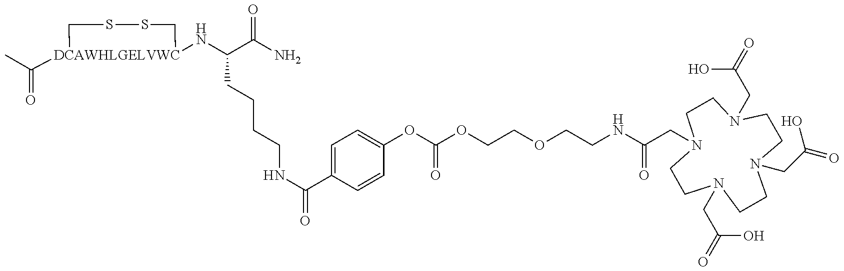 |
| A3K (-carbo- nate- DOTA)- L6Orn (-car- bonate- DOTA) (A3K- L6Orn + 006) | 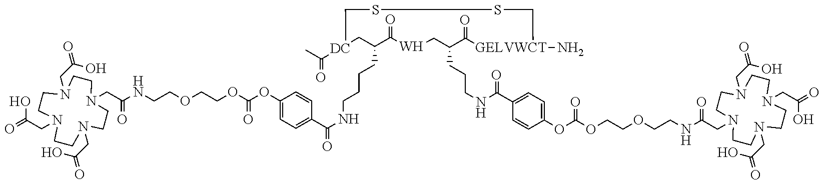 |
| H5Dap (-carbo- nate- DOTA)- E8Q (H5Dap- E8Q + 006) | 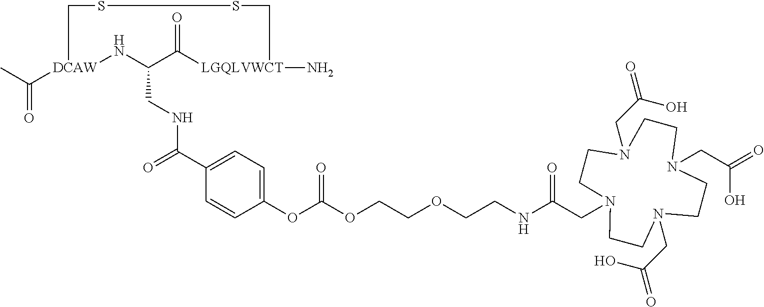 |
| H5Dab- carbonate- DOTA (H5Dab + 006) | 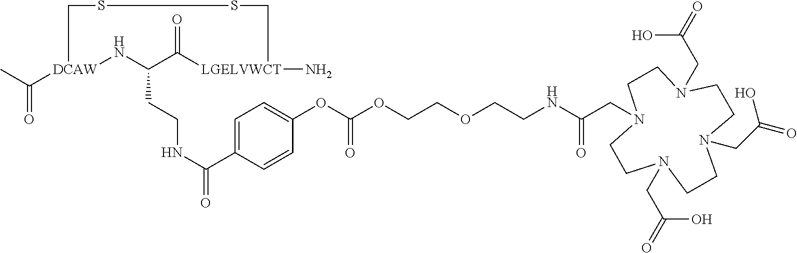 |
| H5Dab (-carbo- nate- DOTA)- E8Q (H5Dab- E8Q + 006) | 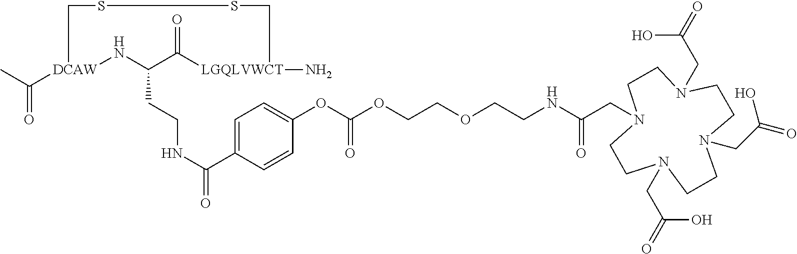 |
| H5Orn (-carbo- nate- DOTA)- E8Q (H5Orn- E8Q + 006) | 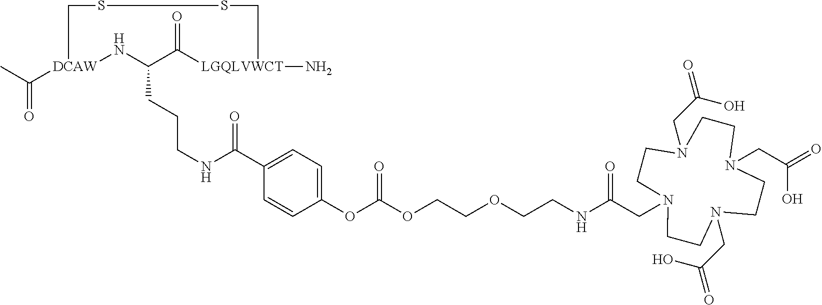 |
| H5K (-carbo- nate- DOTA)- E8Q (H5Orn + 006) | 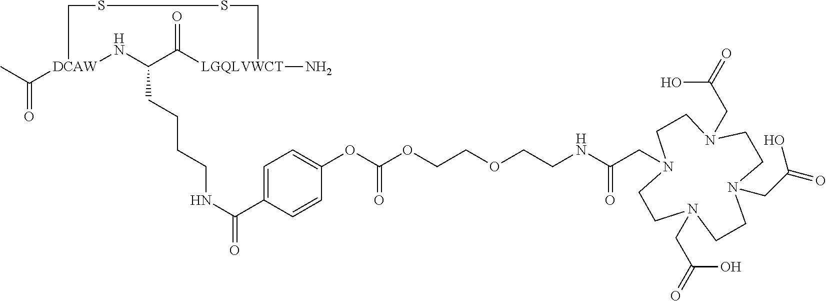 |
| L6Dap- carbonate- DOTA (L6Dap + 006) | 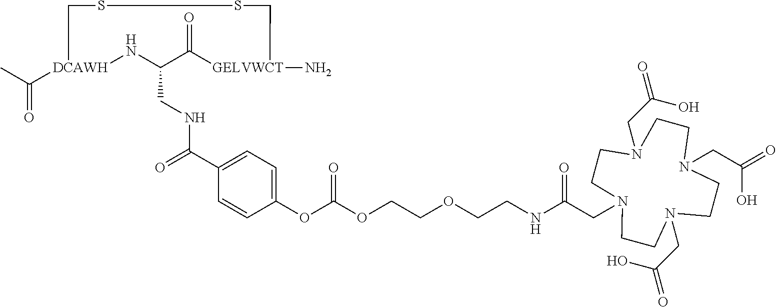 |
| L6Dap (-carbo- nate- DOTA)- E8Q (L6Dap- E8Q + 006) | 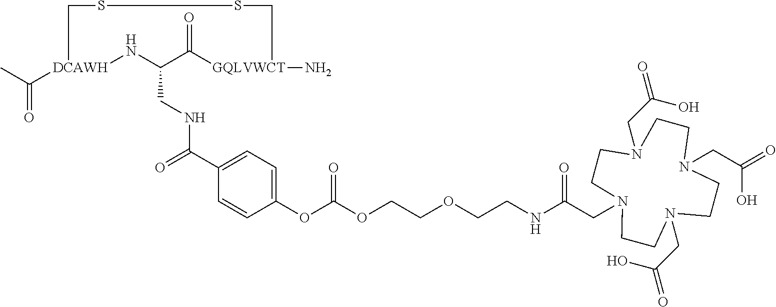 |
| L6Dap- (CH₂)₂- carbonate- DOTA (L6Dap + 007) | 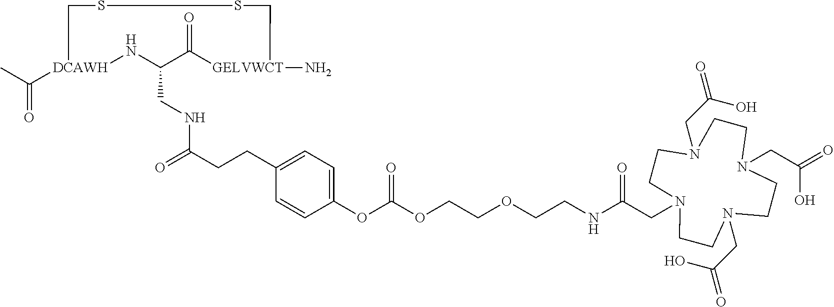 |
| L6Dab- carbonate- DOTA (L6Dab + 006) | 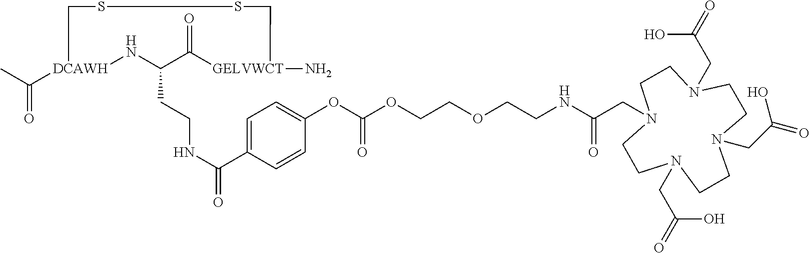 |
| L6Orn- carbonate- DOTA (L6Orn + 006) | 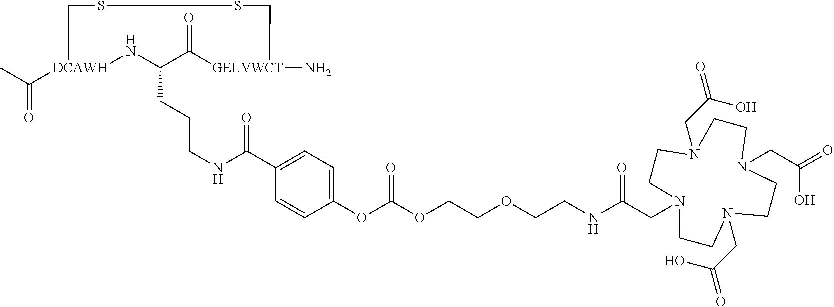 |
| L6-(D- Orn- carbo- nate- DOTA) (L6-(D- Orn) + 006) | 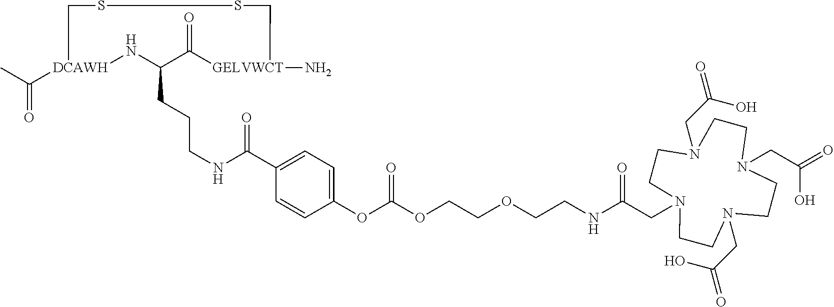 |
| L6-(D- Orn- carbo- nate- DOTA)* (L6-(D- Orn)* + 006) | 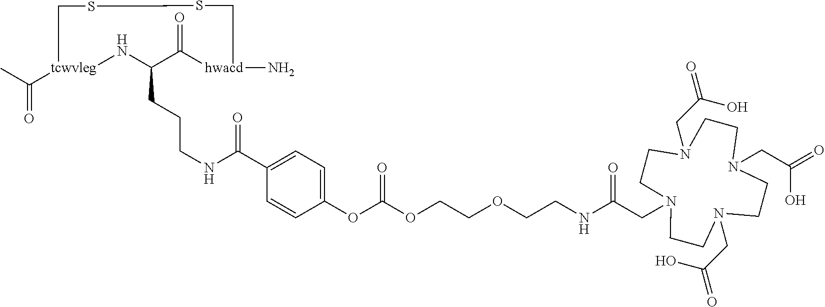 |
| L6Orn- (carbo- nate- DOTA)- E8Q (L6Orn- E8Q + 006) | 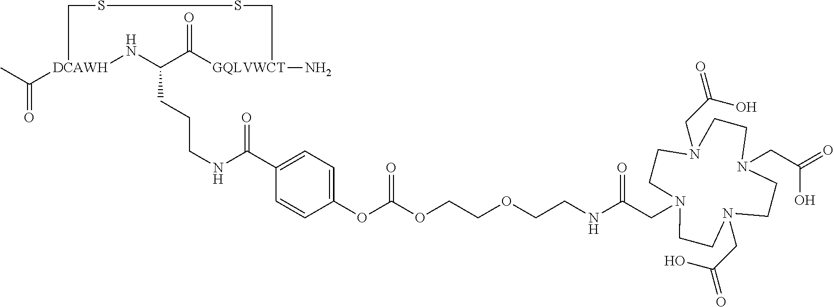 |
| L6K- PEG₁- carbonate- DOTA (L6K- PEG₁ + 006) | 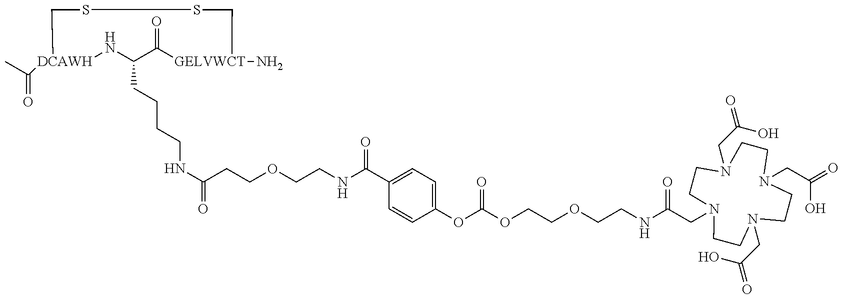 |
| L6K- PEG₂- carbonate- DOTA (L6K- PEG₂ + 006) | 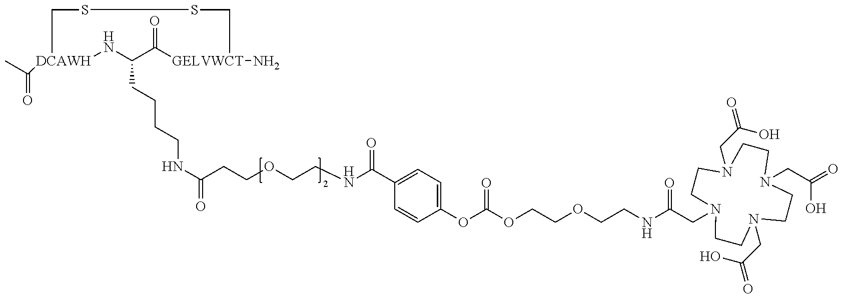 |
| L6K- PEG₃- carbonate- DOTA (L6K- PEG₃ + 006) | 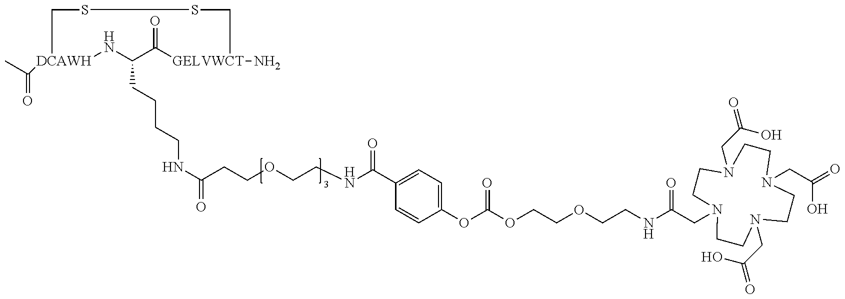 |
| L6K- PEG₁₀- carbonate- DOTA (L6K- PEG₁₀ + 006) | 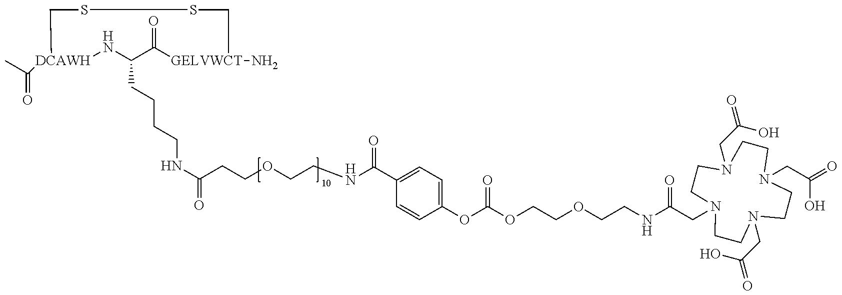 |
| E8Orn- carbonate- DOTA (E8Orn + 006) | 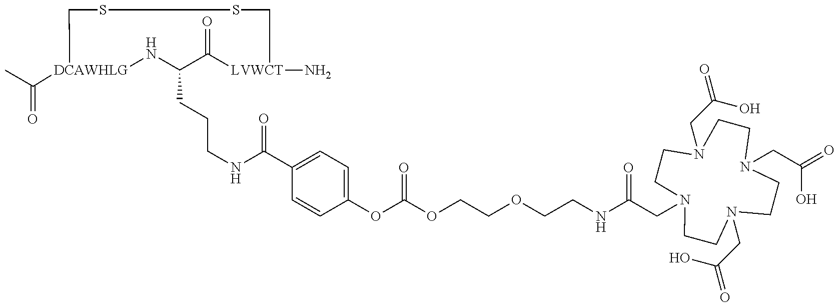 |

*sequence consists of D amino acids

To prepare the reactive conjugates listed in table 5, a solution of carbonate derivative (1.5 eq; compound 006 or 007) in DMF was added to HATU·HPF₆ (1.4 eq) and stirred for 1 min, followed by the addition of DIEA (2 eq). After 3 min, the pre-activated carbonate derivative was added to the Fc-binding vector and the reaction mixture was stirred for 1 to 4 hours at rt. Completion of the reaction was monitored by UPLC-MS. If the reaction did not go to completion, an additional amount of pre-activated carbonate derivative (about 1 eq) was added, and the mixture was further stirred for 1 to 2 hours. The reactive conjugate was precipitated with cold diethyl ether.

Subsequently, the tert-butyl protecting groups of the DOTA moiety were removed by treatment with TFA/TIS/water (95/2.5/2.5, v/v/v) over 1-3 hours at rt, followed by precipitation with cold diethyl ether.

The peptide reactive conjugates were purified by Preparative Reversed Phase-HPLC on a Kinetex® C18 column (100 Å, 5 μm, 150×10.0 mm; Phenomenex Helvetia) using solvent system A (0.1% TFA in water) and B (0.1% TFA in ACN) at a flow rate of 8 mL/min and a gradient in a range of 15-55% of B over 25 min. Peptide elution was monitored at a wavelength of 214 nm. The appropriate fractions were analyzed by UPLC-MS prior to lyophilization.

The purity of the reactive conjugates was determined by UPLC-MS (as described above). The results are shown in the table below.

TABLE 6
Characterization of reactive conjugates

| | Cal- | Cal- | | | | |
|---|---|---|---|---|---|---|
| | culated | culated | Found | Retention | | UPLC- |
| | Exact | [M + | [M + | time | Purity | MS |
| Compound | Mass | 2H]²⁺ | 2H]²⁺ | (min) | (%) | method |
| D1K-carbonate- | 2221.87 | 1111.94 | 1112.00 | 2.394 | 97 | 2 |
| DOTA | | | | | | |
| A3K-carbonate- | 2265.88 | 1133.94 | 1134.00 | 2.362 | 98 | 2 |
| DOTA | | | | | | |
| W4K-carbonate- | 2150.74 | 1076.37 | 1076.40 | 2.219 | 93 | 2 |
| DOTA | | | | | | |
| H5K-carbonate- | 2199.81 | 1100.91 | 1101.00 | 2.491 | 97 | 2 |
| DOTA | | | | | | |
| L6K-carbonate- | 2223.80 | 1112.90 | 1113.34 | 1.53 | 93 | 1 |
| DOTA | | | | | | |
| G7K-carbonate- | 2279.90 | 1140.95 | 1141.00 | 2.403 | 97 | 2 |
| DOTA | | | | | | |
| E8K-carbonate- | 2207.84 | 1104.92 | 1104.90 | 2.4 | 96 | 2 |
| DOTA | | | | | | |
| L9K-carbonate- | 2223.80 | 1112.90 | 1112.90 | 2.229 | 98 | 2 |
| DOTA | | | | | | |
| V10K- | 2237.82 | 1119.91 | 1119.90 | 2.351 | 86 | 2 |
| carbonate- | | | | | | |
| DOTA | | | | | | |
| T13K- | 2235.85 | 1118.93 | 1119.00 | 2.373 | 90 | 2 |
| carbonate- | | | | | | |
| DOTA | | | | | | |
| A3K(-carbonate- | 2903.89 | 1452.95 | 1453.0 | 2.077 | 86 | 2 |
| DOTA)-L6Orn(- | | | | | | |
| carbonate- | | | | | | |
| DOTA) | | | | | | |
| H5Dap(- | 2156.86 | 1079.43 | 1079.60 | 1.71 | 86 | 1 |
| carbonate- | | | | | | |
| DOTA)-E8Q | | | | | | |
| H5Dab- | 2171.85 | 1086.93 | 1087.1 | 1.77 | 93 | 1 |
| carbonate- | | | | | | |
| DOTA | | | | | | |
| H5Dab(- | 2170.86 | 1086.43 | 1086.40 | 2.45 | 69 | 2 |
| carbonate- | | | | | | |
| DOTA)-E8Q | | | | | | |
| H5Orn(- | 2184.86 | 1093.43 | 1093.68 | 1.71 | 90 | 1 |
| carbonate- | | | | | | |
| DOTA)-E8Q | | | | | | |
| H5K(-carbonate- | 2198.86 | 1100.43 | 1100.73 | 1.74 | 91 | 1 |
| DOTA)-E8Q | | | | | | |
| L6Dap- | 2181.86 | 1091.93 | 1091.90 | 2.181 | 68 | 2 |
| carbonate- | | | | | | |
| DOTA | | | | | | |
| L6Dap(- | 2180.81 | 1091.41 | 1091.38 | 1.45 | 97 | 1 |
| carbonate- | | | | | | |
| DOTA)-E8Q | | | | | | |
| L6Dap-(CH₂)₂- | 2209.80 | 1105.90 | 1105.80 | 2.228 | 92 | 2 |
| carbonate- | | | | | | |
| DOTA | | | | | | |
| L6Dab- | 2193.83 | 1097.92 | 1099.00 | 2.183 | 85 | 2 |
| carbonate- | | | | | | |
| DOTA | | | | | | |
| L6Orn- | 2209.83 | 1105.92 | 1106.00 | 2.193 | 98 | 2 |
| carbonate- | | | | | | |
| DOTA | | | | | | |
| L6-(D-Orn- | 2209.83 | 1105.92 | 1105.60 | 1.521 | 91 | 2 |
| carbonate- | | | | | | |
| DOTA) | | | | | | |
| L6-(D-Orn- | 2209.83 | 1105.92 | 1106.00 | 1.583 | 90 | 2 |
| carbonate- | | | | | | |
| DOTA)* | | | | | | |
| L6Orn(- | 2208.81 | 1105.41 | 1105.21 | 1.47 | 96 | 1 |
| carbonate- | | | | | | |
| DOTA)-E8Q | | | | | | |
| L6K-PEG₁(- | 2339.23 | 1170.62 | 1170.46 | 1.51 | 100 | 1 |
| carbonate- | | | | | | |
| DOTA | | | | | | |
| L6K-PEG₂- | 2383.23 | 1192.62 | 1192.40 | 2.209 | 91 | 2 |
| carbonate- | | | | | | |
| DOTA | | | | | | |
| L6K-PEG₃- | 2427.33 | 1214.67 | 1214.24 | 1.53 | 96 | 1 |
| carbonate- | | | | | | |
| DOTA | | | | | | |
| L6K-PEG₁₀- | 2735.20 | 1368.60 | 1368.70 | 2.288 | 93 | 2 |
| carbonate- | | | | | | |
| DOTA | | | | | | |

*sequence consists of D amino acids

### Example 6: Preparation of FITC-carbonate-containing Reactive Conjugates

The Fc-binding vectors prepared in Example 1 were converted into reactive conjugates of formula (1) by coupling of compound 009 to the amino group of the side chain of the respective Fc-binding ligands. Synthesis of compound H5Dab-FITC, is shown in a scheme below. The other FITC-carbonate-peptide conjugates were prepared in a similar way. The FITC-containing reactive conjugates are shown in the table below.

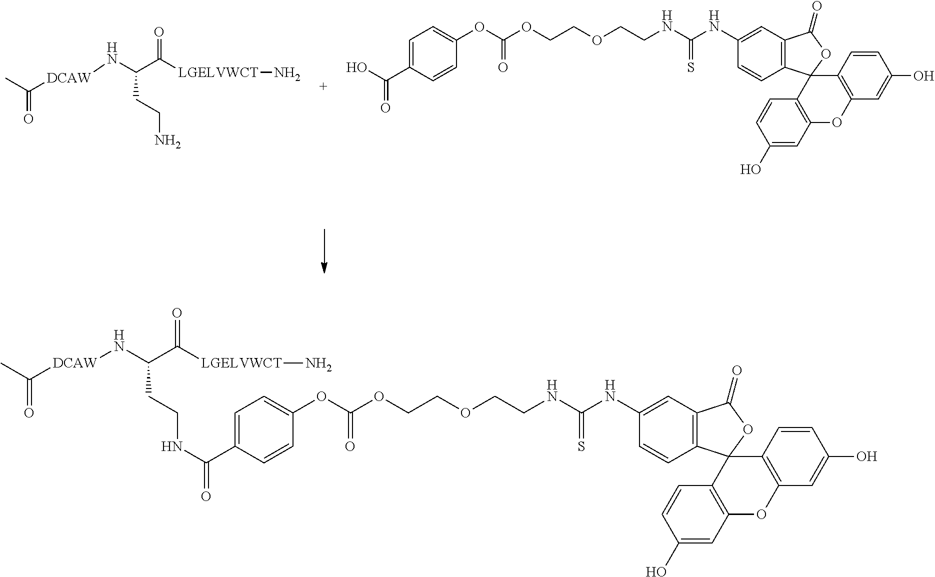

TABLE 7
Structures of FITC-carbonate-containing reactive conjugates

| Com- | |
|---|---|
| pound | Structure |
| H5Dab- carbo- nate- FITC (H5Dab + 009) | 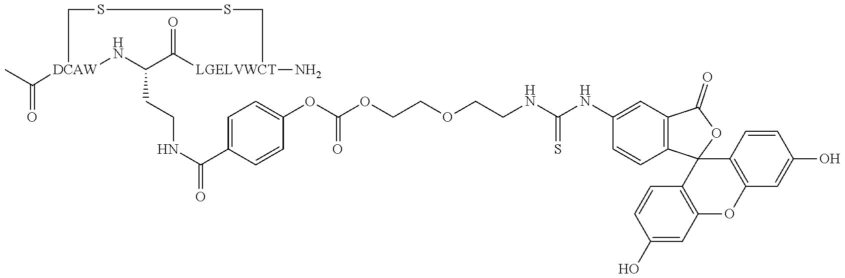 |
| H5K- carbo- nate- FITC (H5K + 009) | 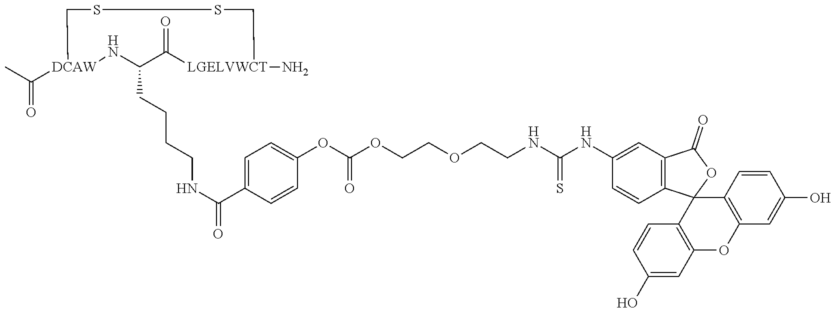 |
| L6K- carbo- nate- FITC (L6K + 009) | 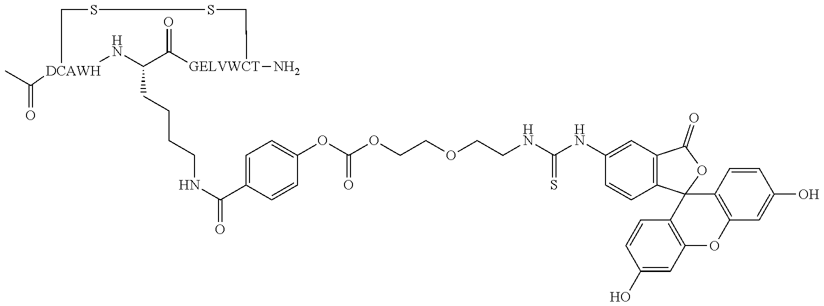 |
| L6Orn- carbo- nate- FITC (L6Orn + 009) | 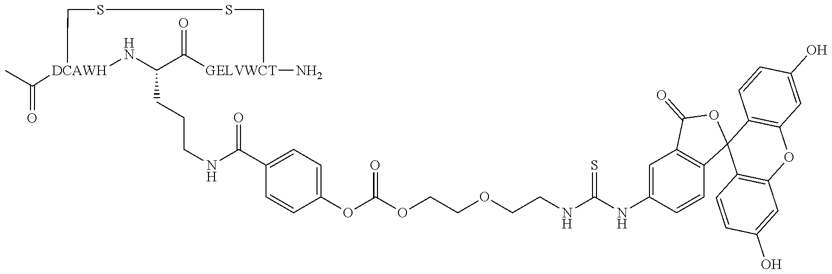 |

To prepare the reactive conjugates, a solution of compound 009 (1.4 eq) in DMF was added to HATU·HPF₆ (1.3 eq) and stirred for 1 min, followed by the addition of DIEA (1.5 eq). After 3 min, the pre-activated carbonate derivative was added to the Fc-binding vector (final concentration of the Fc-binding vector in the reaction mixture was 20-70 mg/ml) and the reaction mixture was stirred for 1 to 4 hours at rt. Completion of the reaction was monitored by UPLC-MS. If the reaction did not go to completion, an additional amount of pre-activated compound 009 (about 1 eq) prepared by mixing compound 009 with HATU·HPF₆ (0.9 eq) and DIEA (1.1 eq) in DMF was added, and the mixture was further stirred for 1 to 2 hours. The reactive conjugate was precipitated with cold diethyl ether and purified by HPLC as described in Example 5.

The purity of the reactive conjugates was determined by UPLC-MS (as described above). The results are shown in the table below.

TABLE 8
Characterization of FITC reactive conjugates

| | Cal- | Cal- | | | | |
|---|---|---|---|---|---|---|
| | culated | culated | Found | Retention | | UPLC- |
| | Exact | [M + | [M + | time | Purity | MS |
| Compound | Mass | 2H]²⁺ | 2H]²⁺ | (min) | (%) | method |
| H5Dab- | 2175.85 | 1088.93 | 1089.00 | 2.22 | 97 | 2 |
| carbonate- | | | | | | |
| FITC | | | | | | |
| H5K- | 2203.85 | 1102.93 | 1102.90 | 2.22 | 99 | 2 |
| carbonate- | | | | | | |
| FITC | | | | | | |
| L6K- | 2227.83 | 1114.92 | 1114.91 | 2 | 99 | 1 |
| carbonate- | | | | | | |
| FITC | | | | | | |
| L6Orn- | 2213.80 | 1107.90 | 1107.40 | 2.727 | 98 | 2 |
| carbonate- | | | | | | |
| FITC | | | | | | |

### Example 7: Preparation of Other Payload-carbonate-containing Reactive Conjugates

The Fc-binding vectors prepared in Example 1 were converted into reactive conjugates by coupling different payloads (DTPA, PCTA, NODA-GA, Bn-NOTA, DOTA-GA, DFO, FITC, FAM, TZ, TCO, N₃, DBCO, BCN, maleimide, DM1, DM4, Vedotin) to the amino group of the side chain of the respective Fc-binding vectors. The structures of these payload-containing reactive conjugates prepared in Example 7 are shown in the table below.

TABLE 9
Structures of other payload-carbonate-containing reactive conjugates

| Compound | Structure |
|---|---|
| L6Orn- carbonate- DTPA | 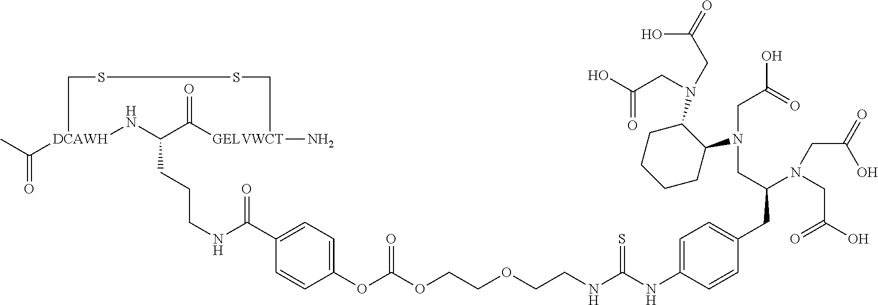 |
| L6Orn- carbonate- PCTA | 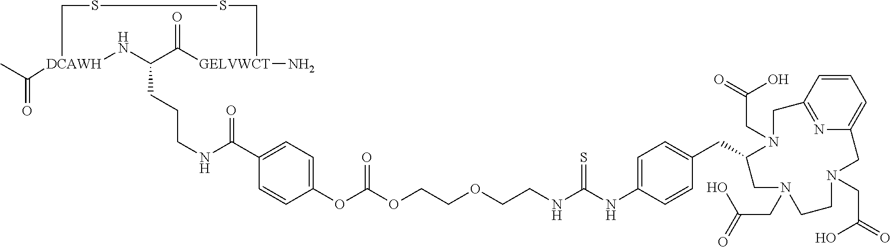 |
| L6Orn- carbonate- NODA-GA | 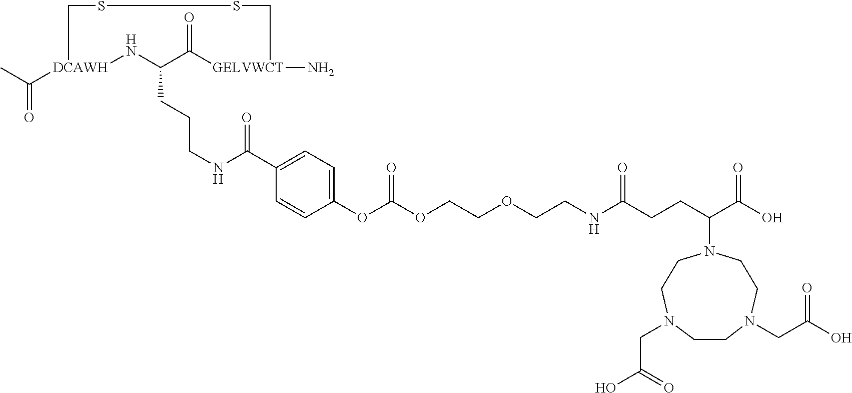 |
| L6Orn- carobnate-Bn- NOTA | 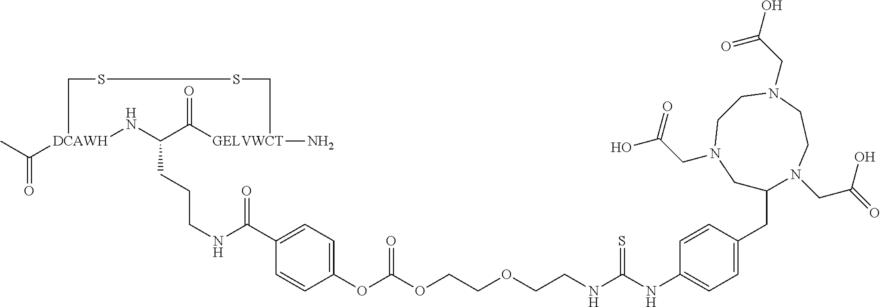 |
| L6Orn- carbonate- DOTA-GA | 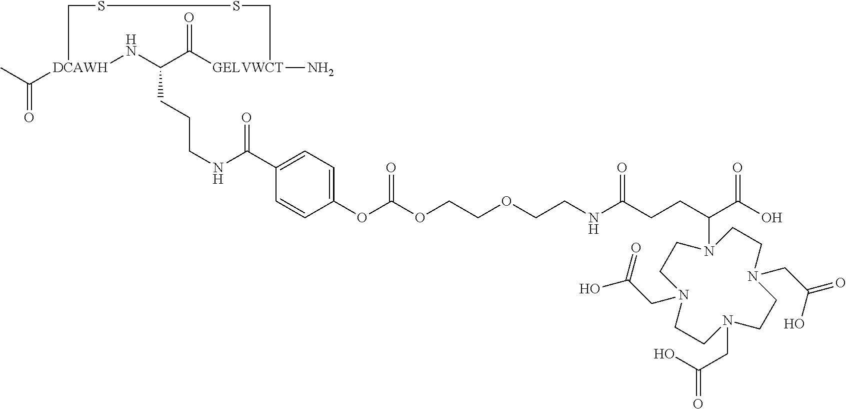 |
| L6Orn- carbonate- DFO | 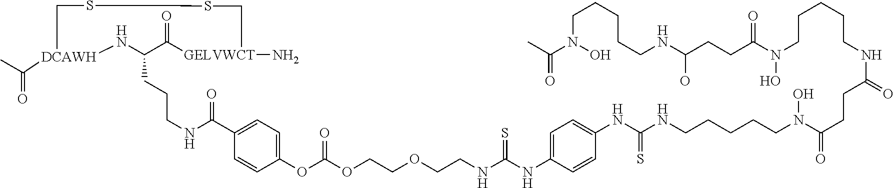 |
| L6Orn- carbonate- triazole-dbco- DFO | 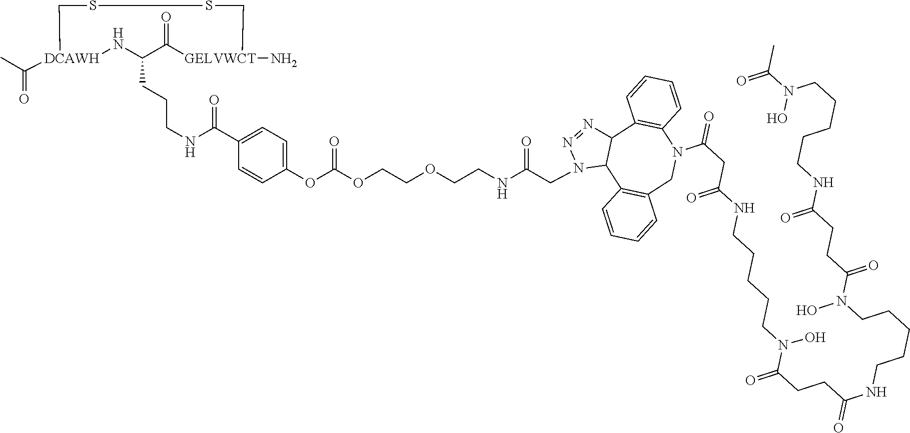 |
| L6Orn- carbonate-S- maleimide- DFO | 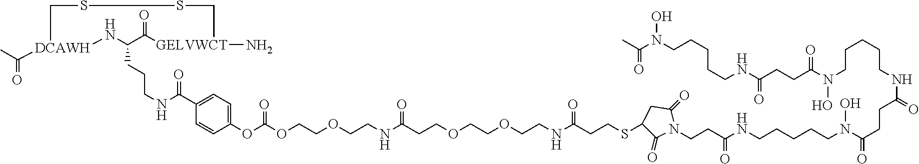 |
| L6Orn- carbonate- FAM^{§} | 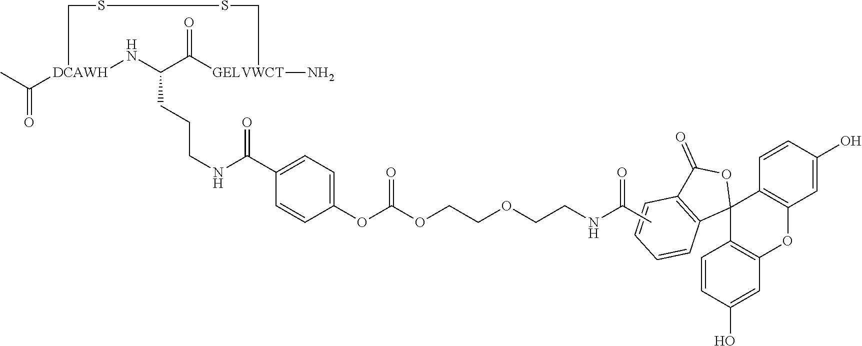 |
| L6Orn- carbonate-TZ | 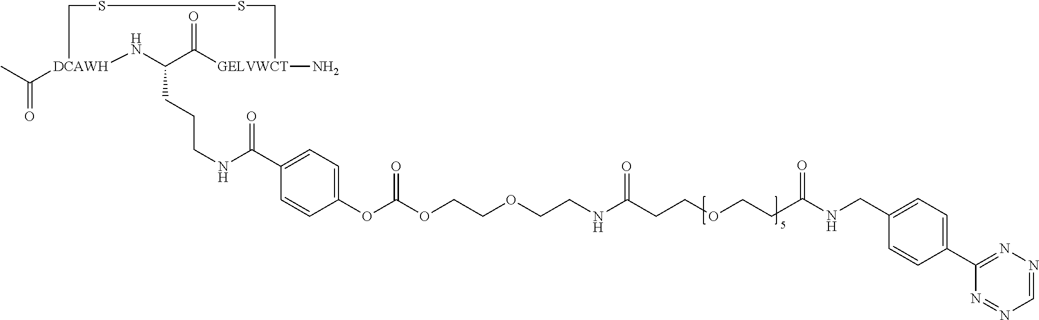 |
| L6Orn- carbonate- TCO | 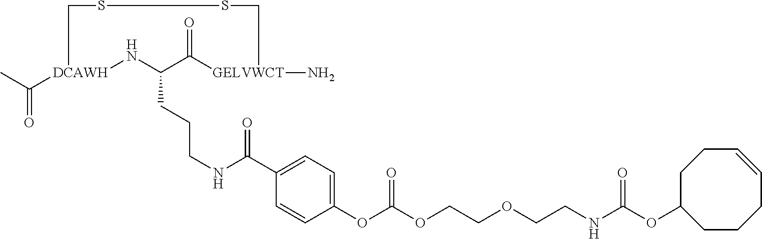 |
| L6Orn- carbonate-N₃ | 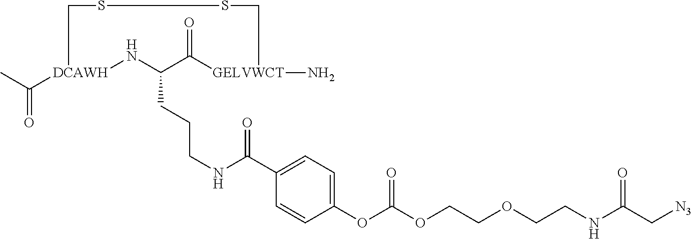 |
| L6Orn- carbonate- DBCO | 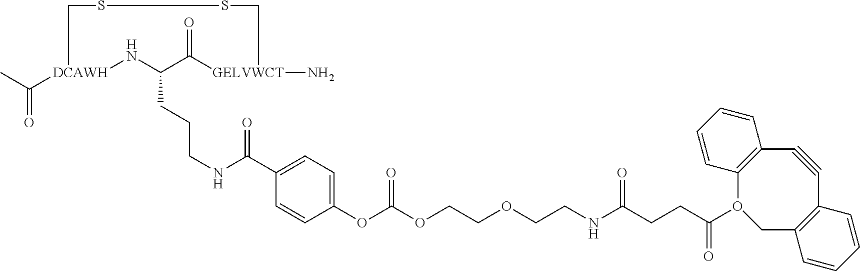 |
| L6Orn- carbonate BCN | 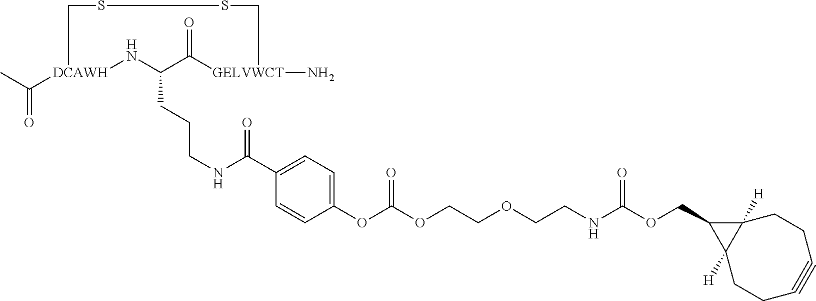 |
| L6Orn- carbonate- maleimide | 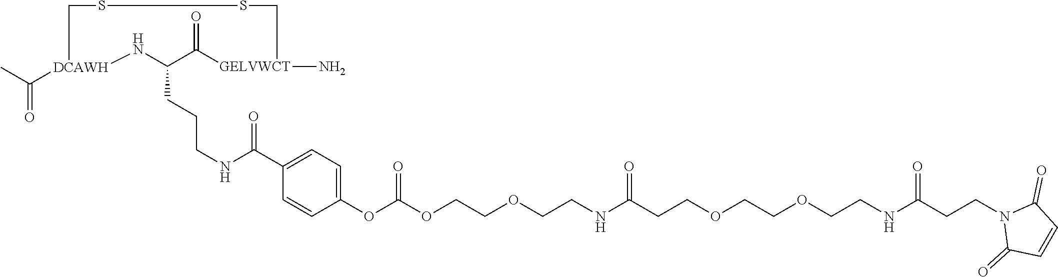 |
| L6Orn- carbonate- MCC-DM1 | 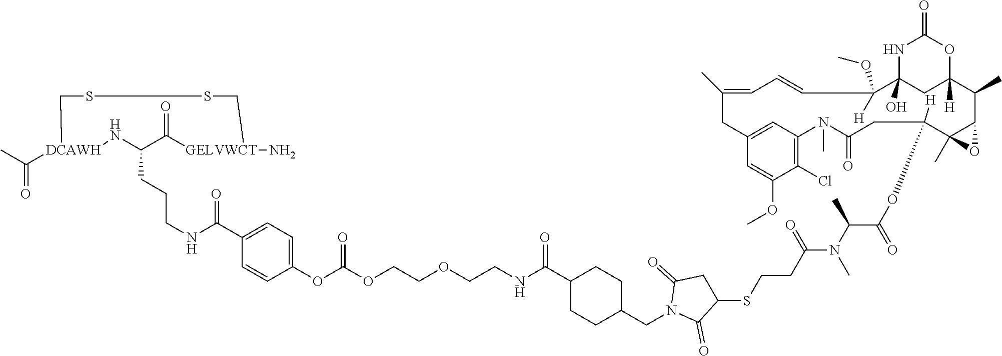 |
| L6Orn- carbonate- SPDB-DM4 | 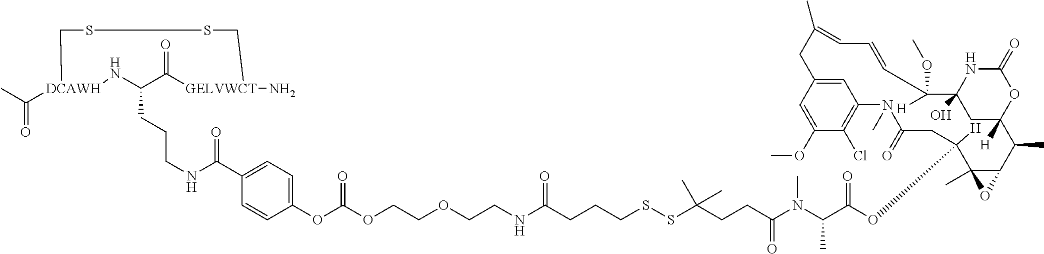 |
| L6Orn- carbonate- diamino- xylene- (SPDB-DM4)₂ | 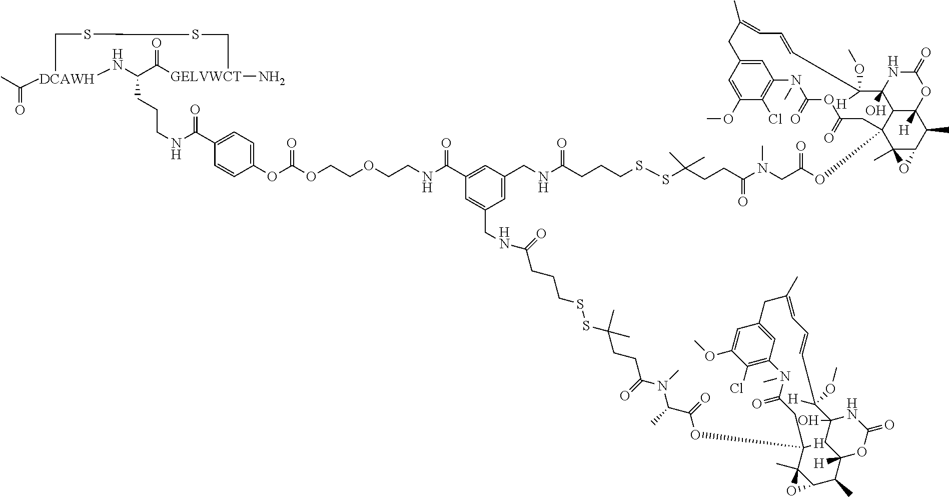 |
| L6Orn- carbonate- Vedotin | 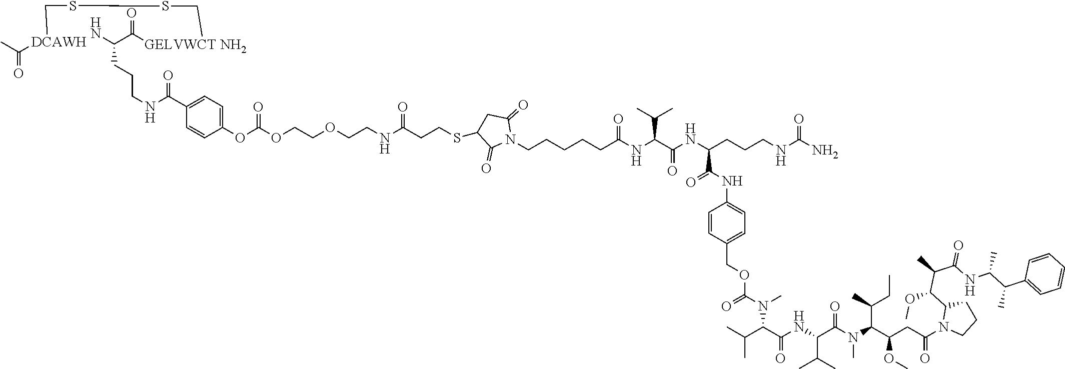 |
| L6Orn- carbonate- (Vedotin)₂ | 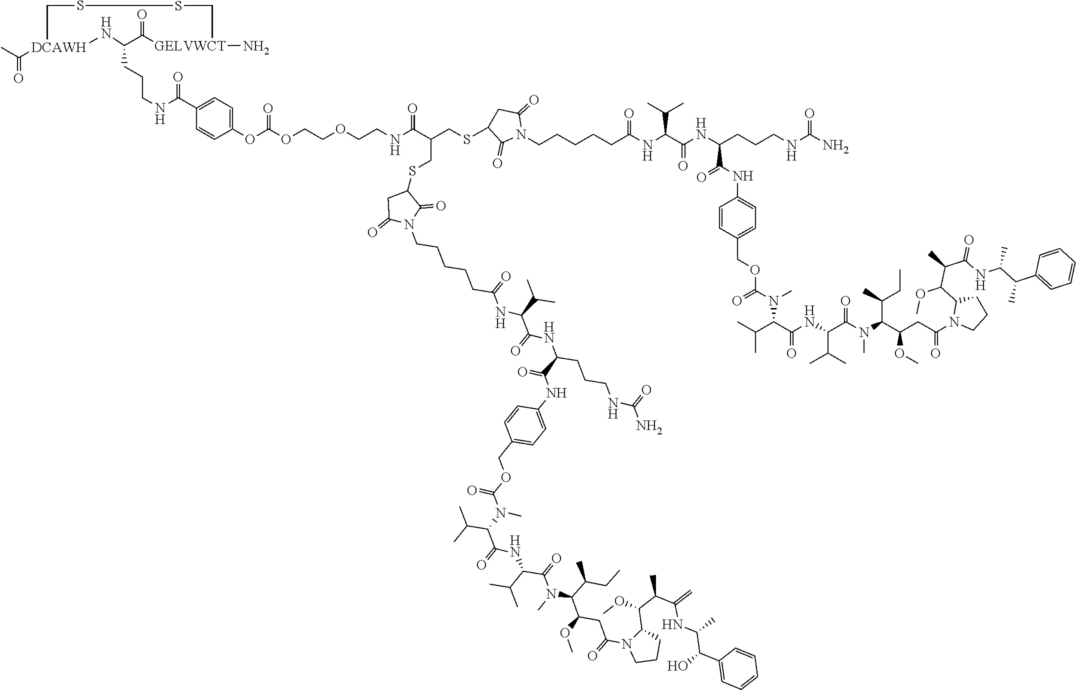 |
| L6Orn- carbonate- multi-DM1 | 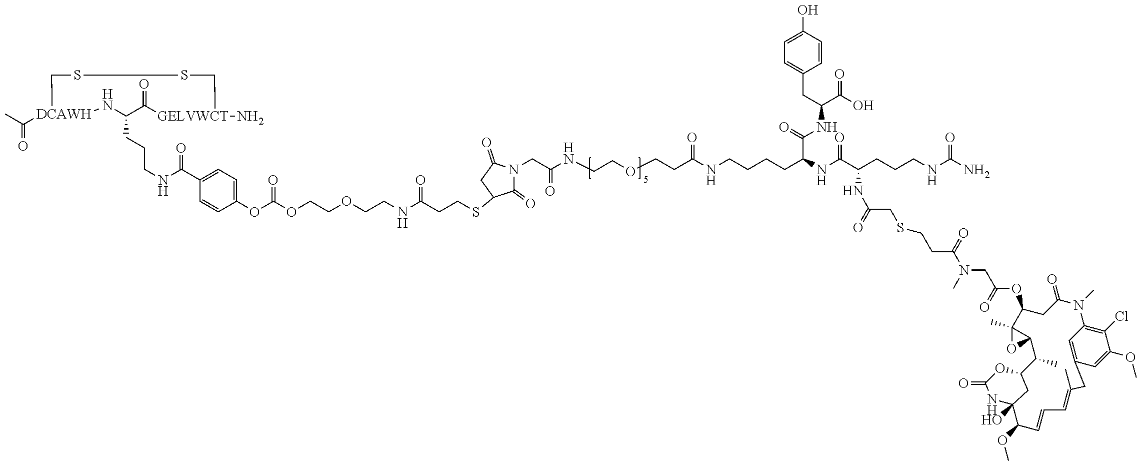 |
| L6Orn- carbonate- multi-(DM1)₂ | 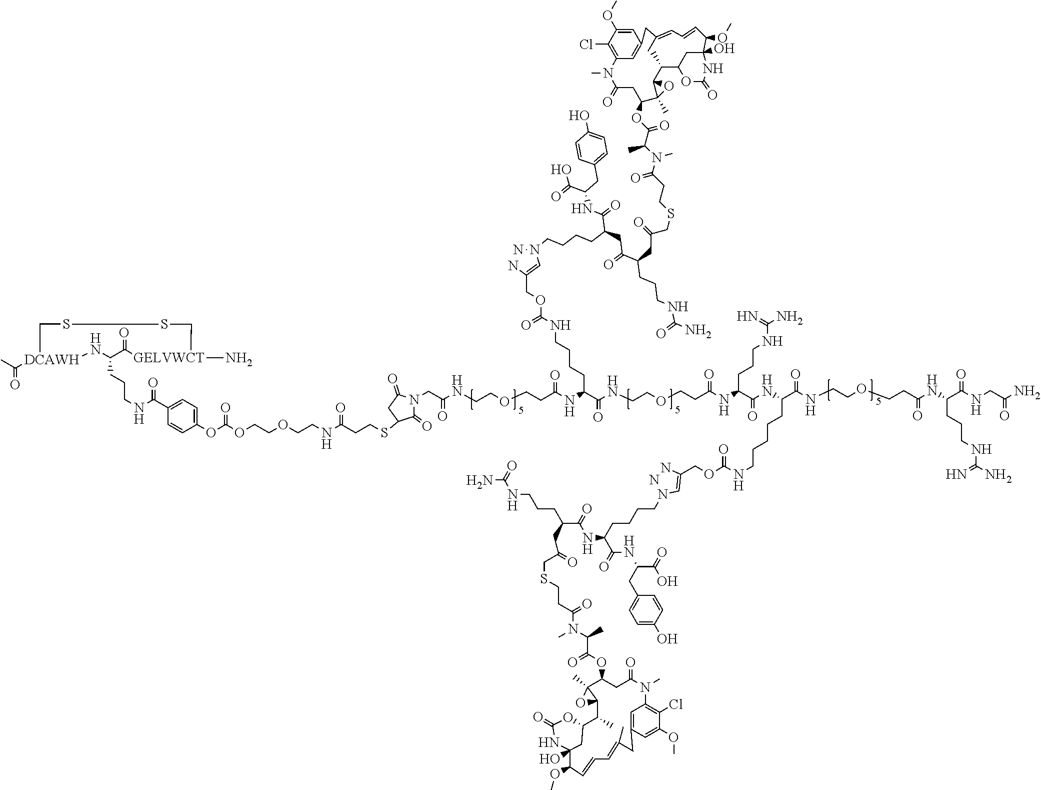 |
| L6Orn- carbonate- PEG₅-Bn- NOTA | 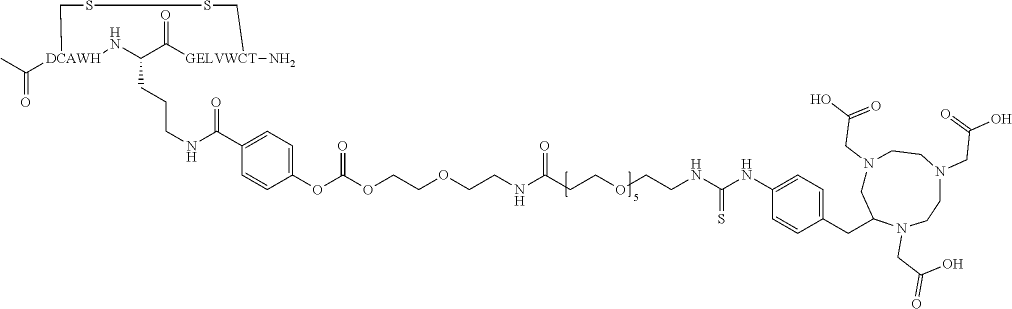 |
| A3hK(-car- bonate- DOTA)- E8Orn(-car- bonate- DOTA) | 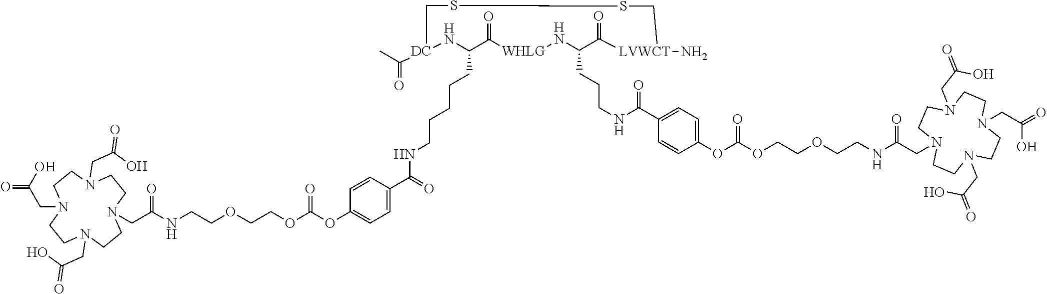 |
| L6Orn- carbonate-Bn- NODAGA | 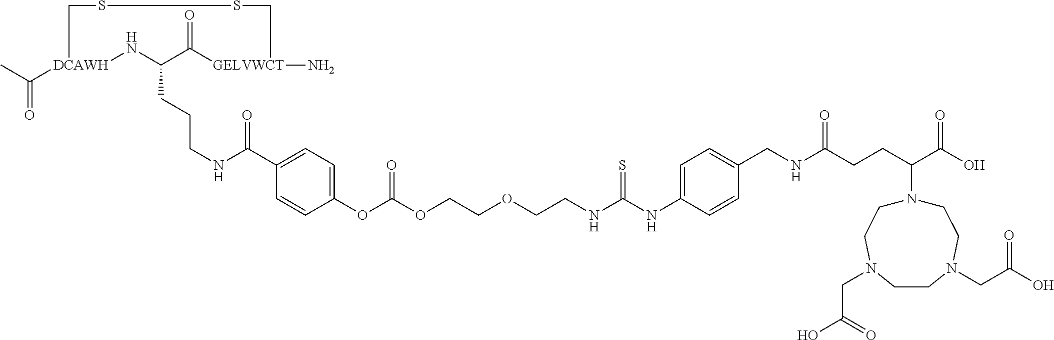 |
| L6Orn- carbonate-Bn- TCMC | 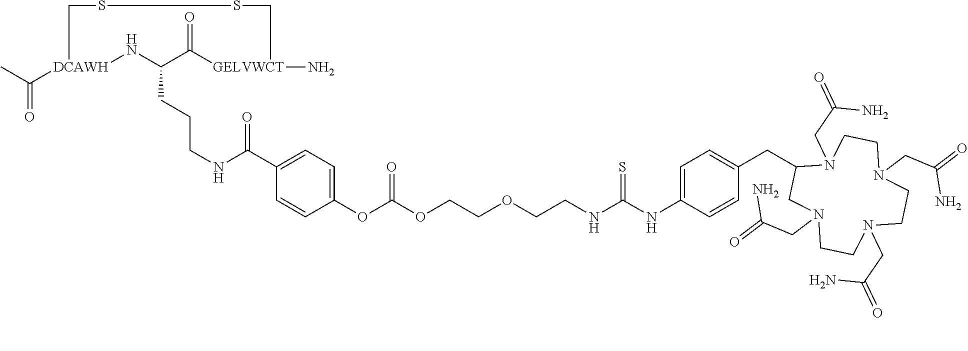 |
| A3hK(-car- bonate-N₃)- E8Orn(-car- bonate-N₃) | 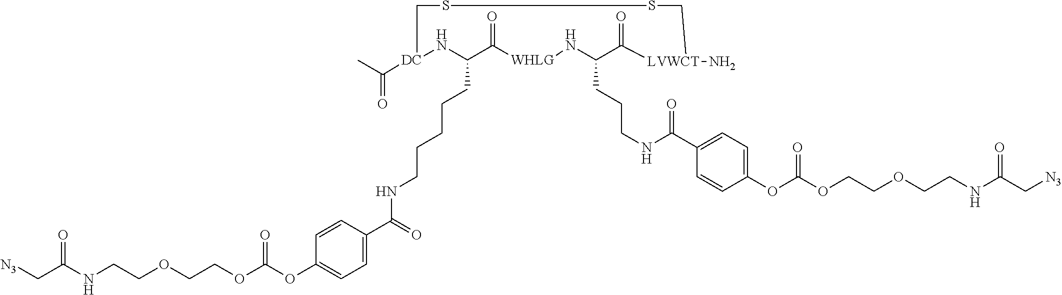 |
| L6Orn- carbonate- mPEG₃₇ | 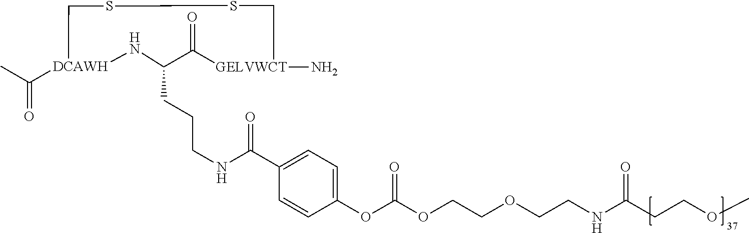 |
| L6Orn- diamino- (mPEG₃₇)₂ | 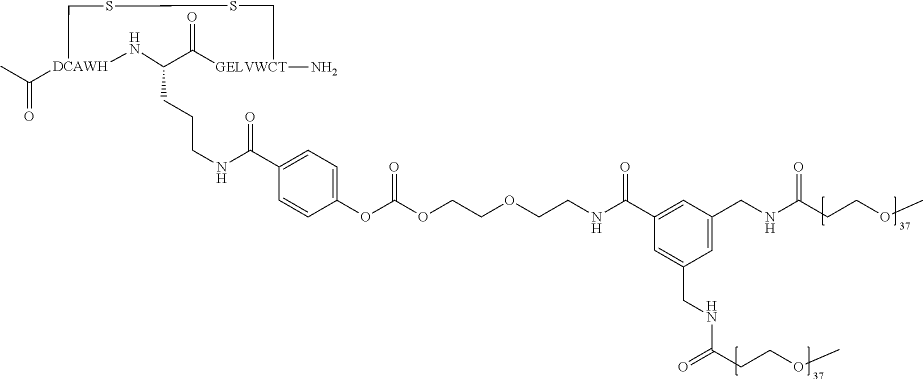 |
| L6Orn- carbonate- PEG₅-PDB- DM4 | 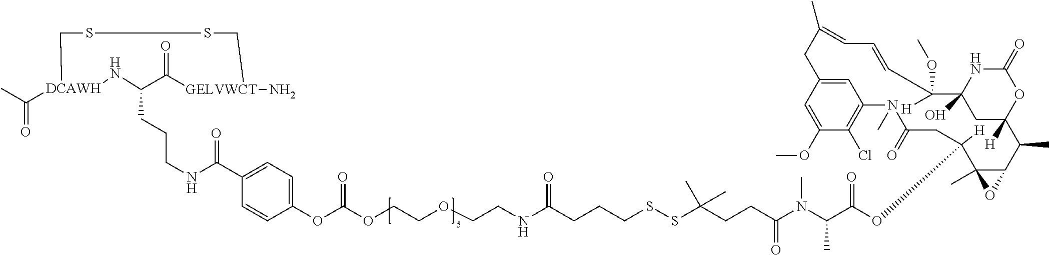 |
| L6Orn(-car- bonate- PDB-DM4)- PEG₂₄ | 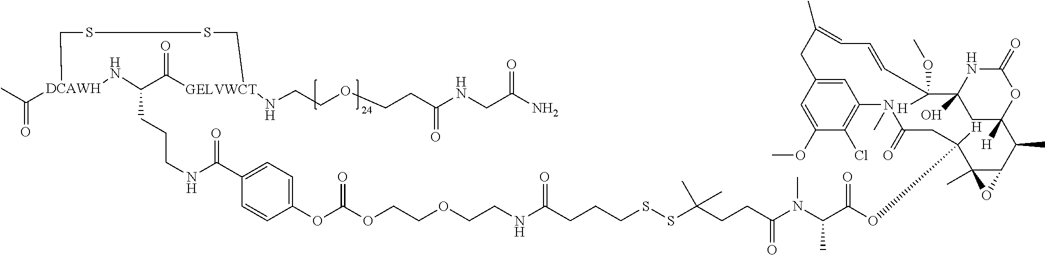 |
| L6Orn(-car- bonate- Vedotin)- PEG₂₄ | 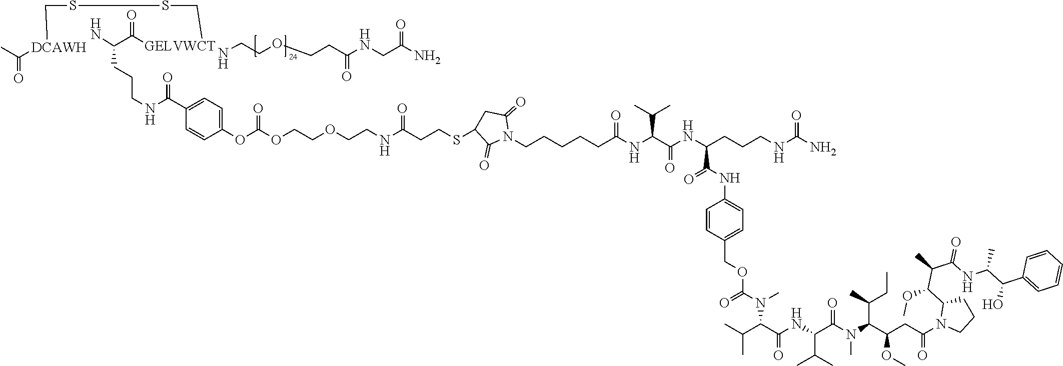 |
| PEG₂₀-L6Orn- carbonate- PDB-DM4 | 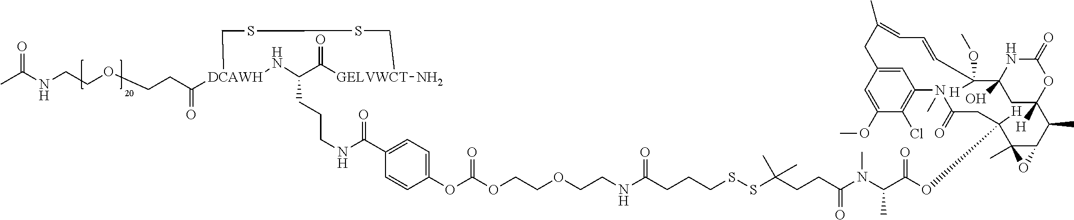 |
| PEG₂₀-L6Orn- carbonate- Vedotin | 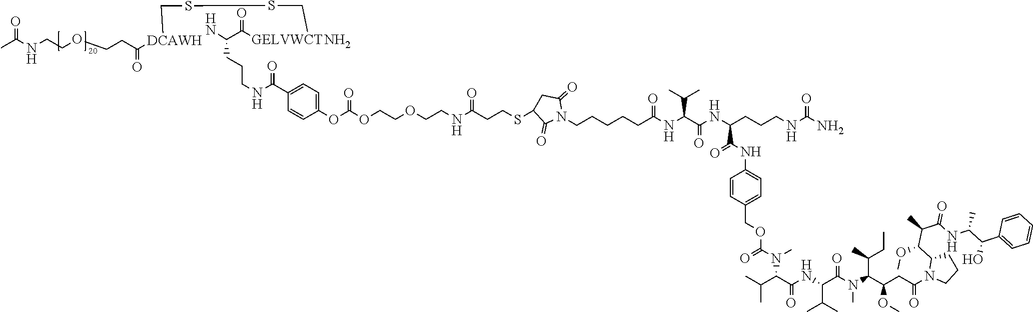 |
| PEG₂₄-L6Orn- carbonate- PEG₃-DM4 | 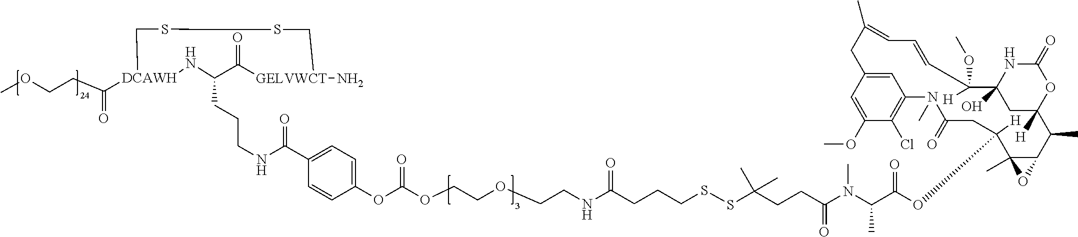 |
| PEG₂₄-L6Orn- carbonate- PEG₃-(suc- VC-PAB- MMAE) | 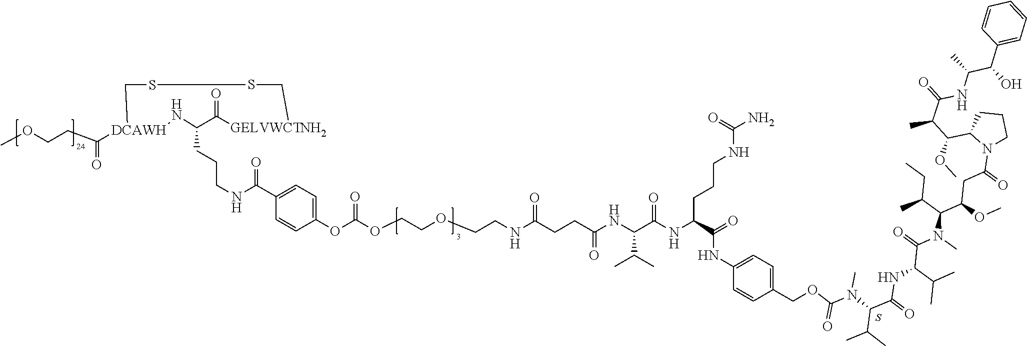 |
| L6Orn- carbonate- suc-VC-PAB- MMAE | 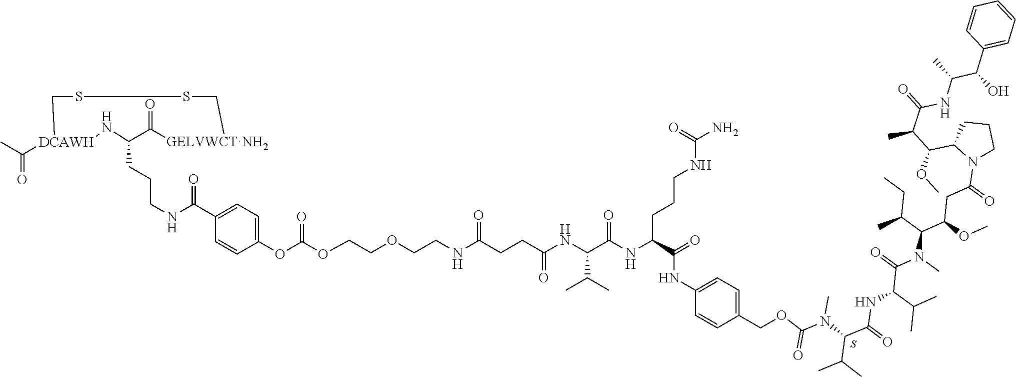 |
| PEG₂₄-L6Orn- carbonate- suc-VC-PAB- MMAE | 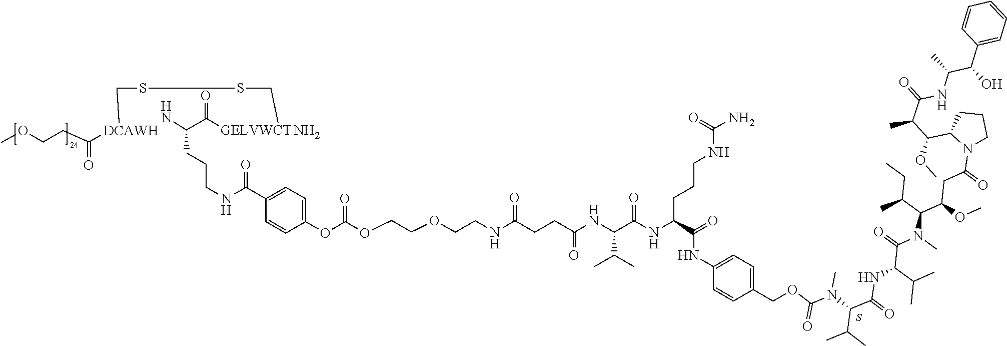 |
| L6Orn- carbonate- PNU | 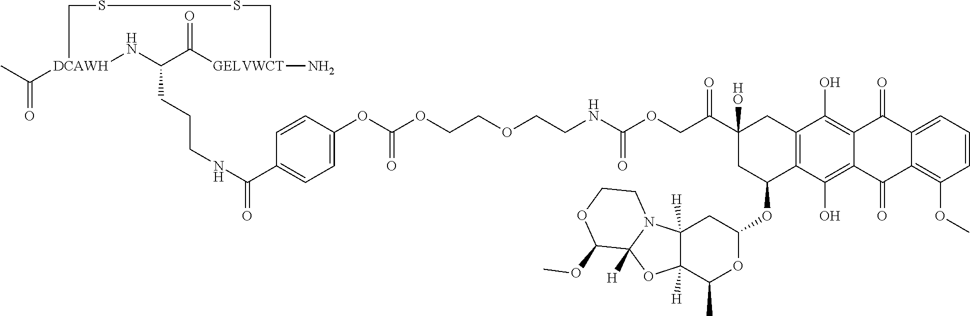 |
| PEG₂₄-L6Orn- carbonate- PNU | 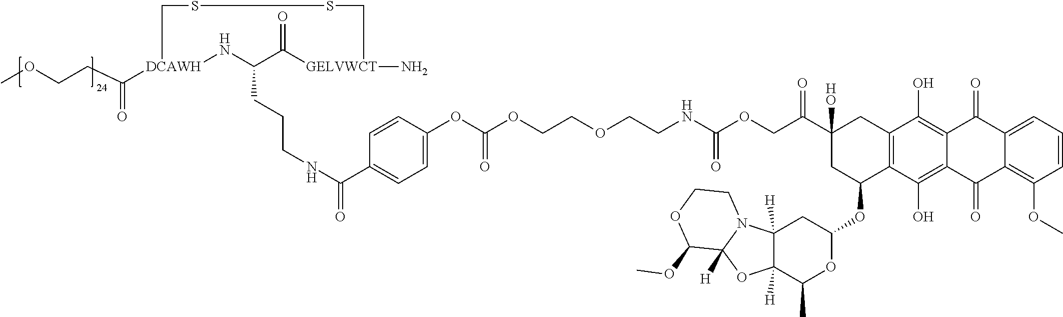 |
| PEG₂₀-L6Orn- carbonate- multi-(DM1)₂ | 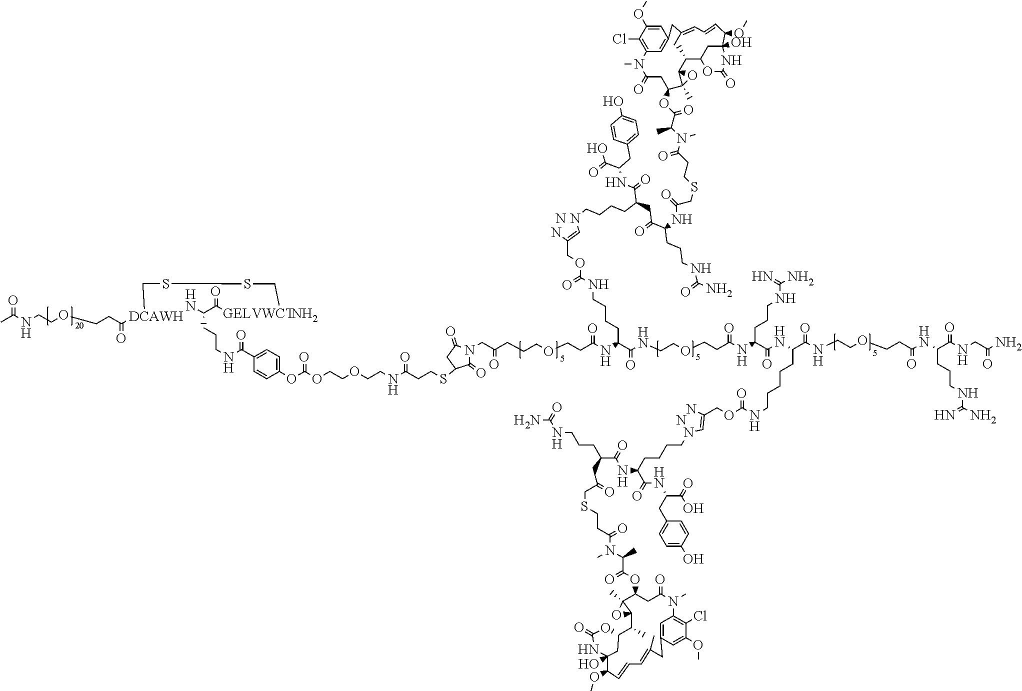 |
| L6Orn- carbonate- multi-(DM1)₂- PEG₁₅ | 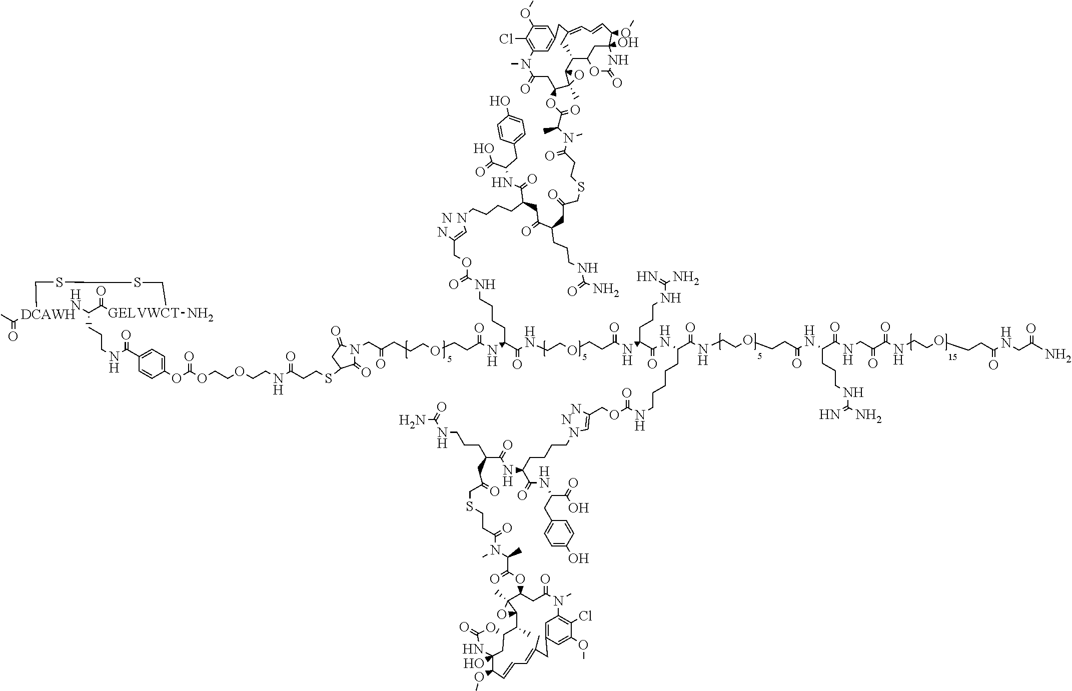 |
| L6Orn- carbonate- multi-(DM1)- K(PEG₂₄)- DM1) | 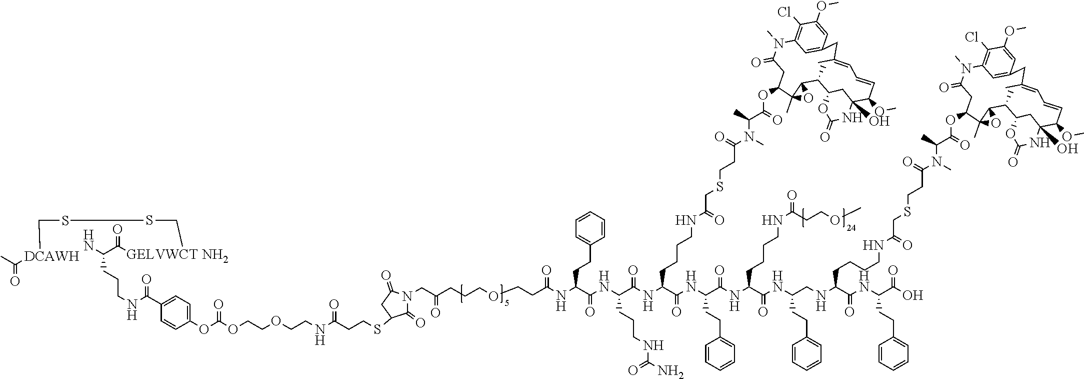 |
| RGNCAYHK (NHS-N₃- DBCO-VC- PAB- MMAE)GQIIW CTYH | 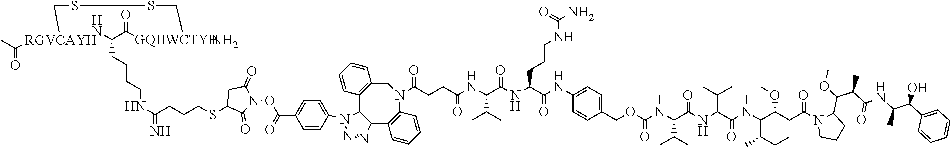 |
| RGNCAYHOrn (NHS-N₃- DBCO-VC- PAB-MMAE) GQIIWCTYH | 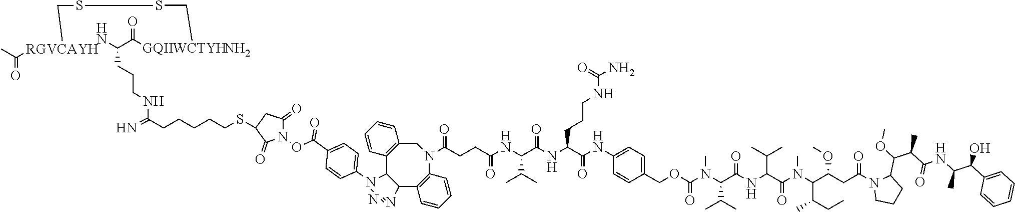 |
| RGNCAYHK (NHS-PEG₁- VC-PAB- MMAE)GQIIW CTYH | 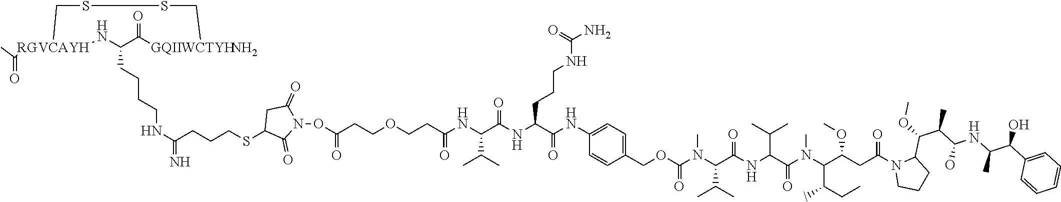 |
| RHNCAYHK (carbonate-N₃- DBCO-VC- PAB- MMAE)GQIIW CTYH | 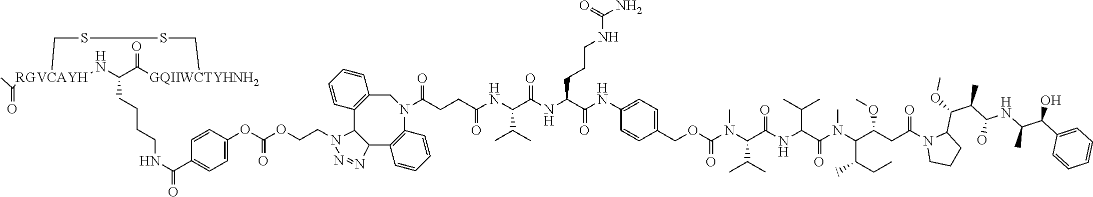 |
| RGNDAYHK (carbonate-suc- VC-PAB- MMAE)GQIIW CTYH | 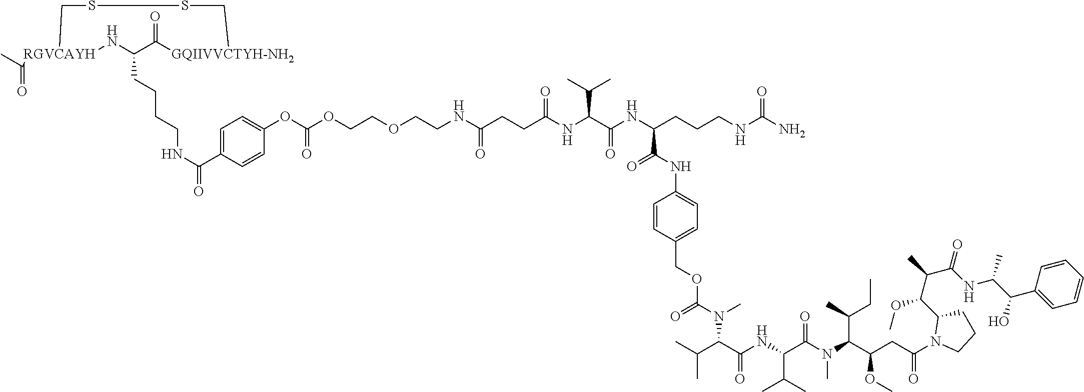 |

^{§}FAM is a mixture of 5-FAM and 6-FAM regioisomers

### Preparation of L6Orn-carbonate-NH₂

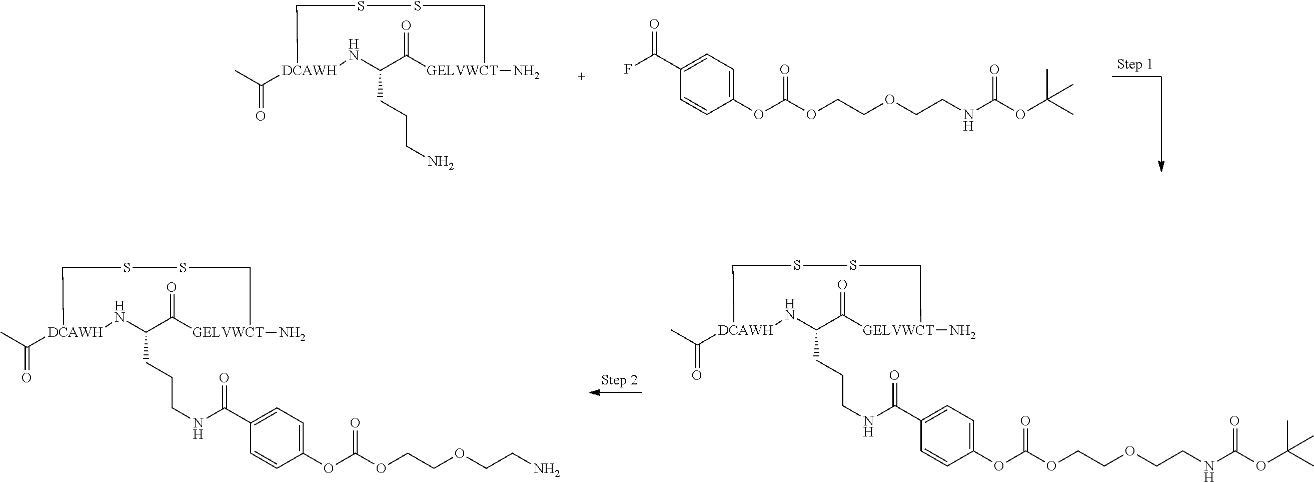

Step 1. 2-[2-(tert-butoxycarbonylamino)ethoxy]ethyl (4-fluorocarbonylphenyl) carbonate (71 mg, 0.19 mmol, 2.0 eq.) in solution in DMF (stock solution 1 mg/40 μL) was added to L6Orn-NH₂ (150 mg, 1.0 eq.) at rt. After stirring at rt for 1 min, DIEA (30 μL, 0.19 mmol, 2.0 eq.) was added to the reaction mixture. After stirring at rt for 10 min, the reaction mixture was concentrated in vacuo. Purification by preparative HPLC (5% to 95% of ACN+0.1% FA of in water+0.1% FA over 24 min) afforded L6Orn-carbonate-NHBoc (117 mg, 61 μmol, 90% UV purity, 57% yield). UPLC-MS (method 4): Rt=2.48 min, m/z=962 [M+2H]²⁺, 960 [M-2H]²⁻.

Step 2. TFA (2.1 mL) was added to L6Orn-carbonate-NHBoc (117 mg, 61 μmol, 1.0 eq.) at rt. After stirring at rt for 5 min, the reaction mixture was concentrated in vacuo. The residue was dissolved in a mixture of ACN (2 mL) and H₂O (2 mL) and freeze-dried to afford L6Orn-carbonate-NH₂ (131 mg, 90% UV purity) as a white powder. UPLC-MS (method 4): Rt=1.77 min, m/z=913 [M+2H]²⁺, 911 [M-2H]²⁻.

### Preparation of L6Orn-carbonate-diaminoxylene

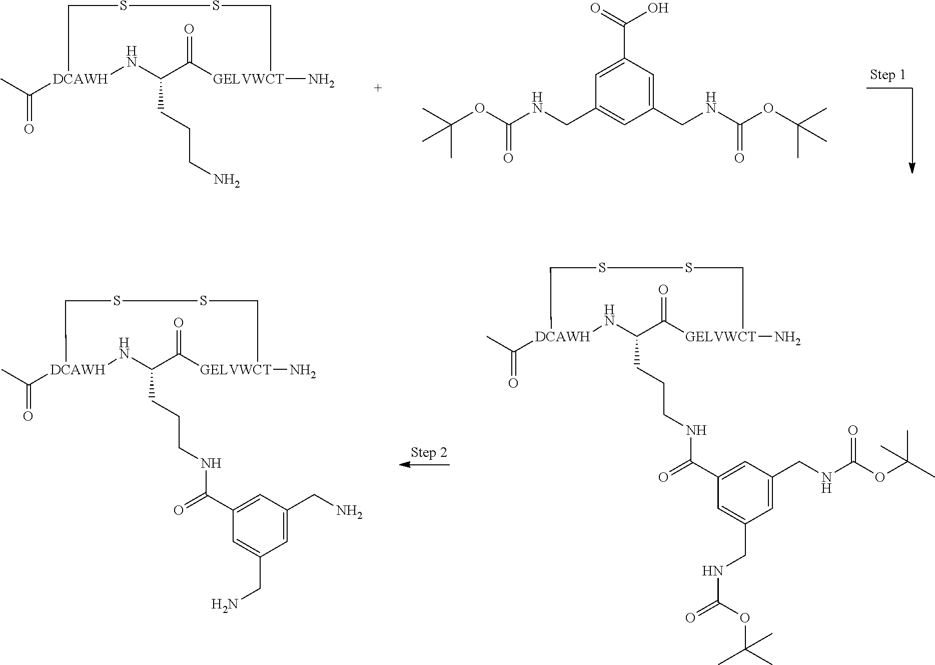

Step 1. HATU·HPF₆ (8.1 mg, 21.4 μmol, 1.3 eq.) was added to a solution of DIEA (11.5 μL, 65.8 μmol, 4.0 eq.) and 3,5-Bis-(((tert-butoxycarbonyl)amino)methyl)benzoic acid (9.9 mg, 24.7 μmol, 1.5 eq.) in DMF (0.6 mL) at rt. After stirring at rt for 7 min, L6Orn-carbonate-NH₂ (30.0 mg, 16.4 μmol, 1.0 eq.) was added to the reaction mixture. After stirring at rt for 55 min, TFA was added dropwise until acidic pH was reached. Purification by reversed phase automated flash chromatography on a C18 biotage column (30 g, 20 to 80% of ACN+0.1% TFA in water+0.1% TFA over 12 CV) afforded L6Orn-carbonate-di(boc-amino)xylene (16.7 mg, 7.6 μmol, 46% yield) as a white lyophilizate. UPLC-MS (method 4): Rt=2.99 min, m/z=1043 [M-Boc+2H]²⁺, 1091 [M-2H]²⁻.

Step 2. TFA (2 mL) was added to a solution of L6Orn-carbonate-di(boc-amino)xylene (16.7 mg, 7.6 μmol, 1.0 eq.) in DCM (2 mL) at rt. After stirring at rt for 12 min, the reaction mixture was concentrated under vacuo. A mixture of ACN/H₂O (1:1, 18 mL) was added and the mixture was freeze-dried to afford L6Orn-carbonate-diaminoxylene (19.3 mg, 7.6 μmol, UV purity 78%, 99% yield) as a white powder. UPLC-MS (method 4): Rt=1.79 min, m/z=662 [M+3H]³⁺, 992 [M-2H]²⁻.

### Preparation of L6Orn-carbonate-S-Mmt

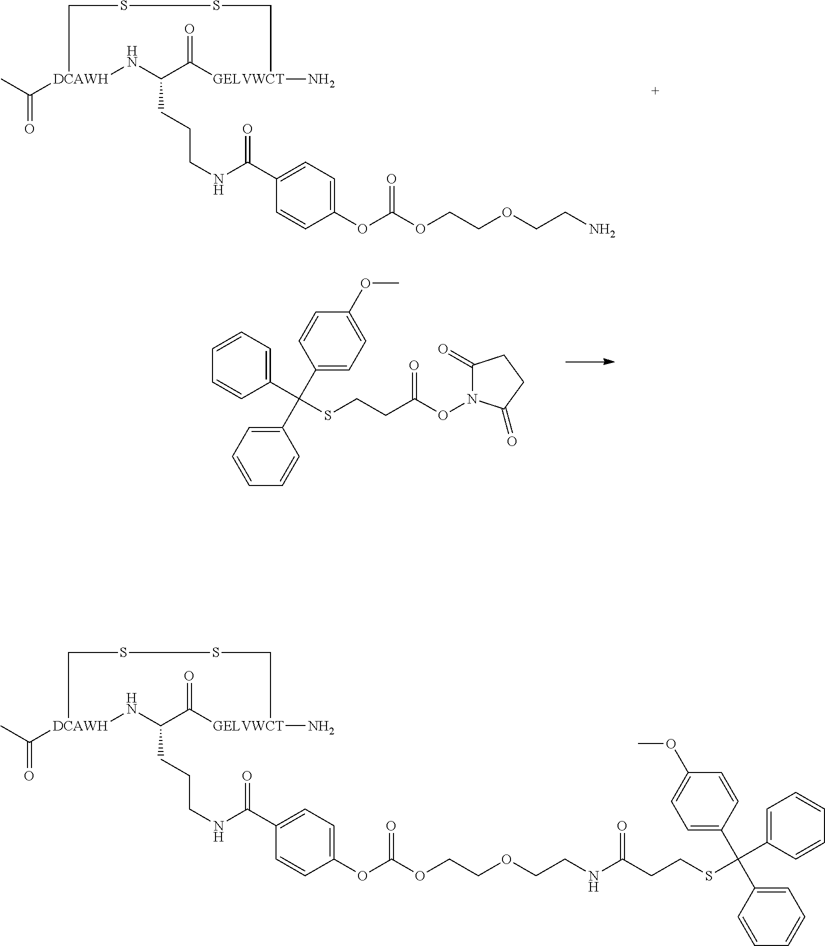

DIEA (21 μL, 123 μmol, 4.0 eq.) was added to a mixture of compound 005 (14.6 mg, 30.7 μmol, 1.0 eq.) and L6Orn-carbonate-NH₂ (81.1 mg, 30.7 μmol, 1.0 eq.) in DMF (1.6 mL) at rt. After stirring at rt for 2 h, more of DIEA (10 μL, 61 μmol, 2.0 eq) was added to the reaction mixture. After stirring at rt for 3 h, more of compound 005 (7.3 mg, 15.4 μmol, 0.5 eq) was added to the reaction mixture. After stirring at rt for 30 min, TFA was added until acidic pH was reached. Purification by reversed phase preparative HPLC (5 to 100% of ACN+0.1% FA in water+0.1% FA) afforded L6Orn-carbonate-S-Mmt (43.7 mg, 19.0 μmol, UV purity 95%, 62% yield) as a white solid after freeze-drying. UPLC-MS (method 4): Rt=3.13 min, m/z=1104 [M+H+Na]²⁺, 1090 [M-2H]²⁻.

### Preparation of L6Orn-carbonate-PEG₅-NH₂

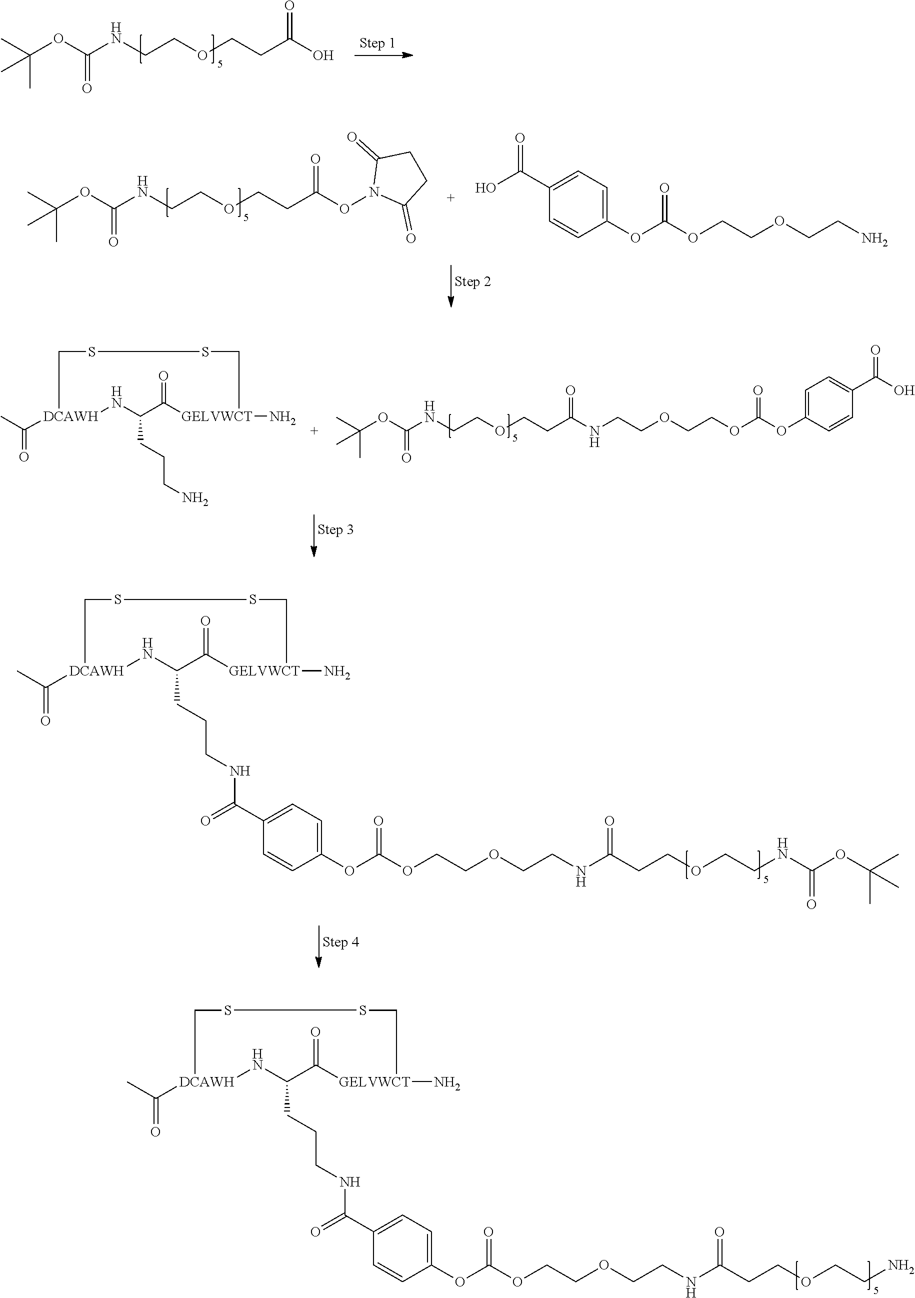

Step 1. EDC·HCl (84.20 mg, 493.2 μmol, 2.7 eq.) followed by N-hydroxysuccinimide (50.50 mg, 438.8 μmol, 2.7 eq.) were added to a solution of Boc-NH-PEGs-COOH (66.00 mg, 161.2 mmol, 1.0 eq.) in DCM (1.5 mL). The reaction mixture was stirred at rt for 2 h, then concentrated in vacuo. Purification by preparative HPLC (10 to 70% of ACN+0.1% TFA in water+0.1% TFA) afforded Boc-NH-PEGs-OSu (65.20 mg, 128.7 μmol, 100% UV purity, 80% yield) as a colourless oil after freeze-drying. UPLC-MS (method: 3): Rt=1.43 min m/z=451 [M-(tBu)+H]*, 408 [M-Boc+H]*, 505 [M−H]⁻, 551 [M+FA-H]⁻; ¹H NMR (ODCl₃, 400 MHz) δ 5.04 (br s, 1H), 3.84 (t, J=6.5 Hz, 2H), 3.65−3.59 (m, 16H), 3.53 (t, 2H), 3.30 (br s, 2H), 2.90 (t, J=6.5 Hz, 2H), 2.83 (br s, 4H), 1.44 (s, 9H); 13C NMR (CDCl₃, 100 MHz) δ 169.3, 167.0, 156.4, 79.6, 71.0, 70.98, 70.93, 70.91, 70.86, 70.84, 70.60, 70.55, 66.0, 32.5, 28.8, 25.9.

Step 2. DIEA (54 μL, 314.4 μmol, 2.0 eq.) was added to a solution of Boc-NH-PEG₅-OSu (65.20 mg, 159.2 μmol, 1.0 eq.) and 4-(((2-(2-aminoethoxy)ethoxy)carbonateonyl)oxy)benzoic acid (42.60 mg, 158.2 μmol, 1.0 eq.) in DMF (1.4 mL). After stirring at rt for 3 h, TFA was added until acidic pH was reached. Purification by preparative HPLC (5 to 70% of ACN+0.1% TFA in water+0.1% TFA) afforded HOOC-carbonate-PEG₁-CO-PEG₅-NH-Boc (59.00 mg, 89.3 μmol, 100% UV purity, 56% yield) as a colourless oil after freeze-drying. UPLC-MS (method 3): Rt=1.45, m/z=661 [M+H]*, 561 [M-Boc+H]*, 659 [M−H]—; ¹H NMR (CDCl₃, 400 MHz) δ 8.06 (d, J=8.7 Hz, 2H), 7.22 (d, J=8.7 Hz, 2H), 6.86 (br s, 1H), 5.07 (br s, 1H), 4.36-4.34 (m, 2H), 3.71-3.69 (m, 2H), 3.67 (t, J=5.8 Hz, 2H), 3.58-3.53 (m, 18H), 3.47 (t, J=5.2 Hz, 2H), 3.42 (q, J=5.4 Hz, 2H), 2.44 (t, J=5.8 Hz, 2H), 1.37 (s, 9H); ¹³C NMR (CDCl₃, 100 MHz) δ 172.5, 169.0, 154.7, 153.0, 131.8, 127.8, 121.0, 79.3, 70.54, 70.51, 70.48, 70.32, 70.23, 69.73, 68.49, 67.78, 67.1, 39.3, 36.6, 28.4.

Step 3. HATU·HPF6 (10.74 mg, 28.2 μmol, 1.1 eq.) followed by DIEA (20 μL, 114.8 μmol, 4.5 eq.) were added to a solution of HOOC-carbonate-PEG₁-CO-PEG₅-NH-Boc (20.37 mg, 30.8 μmol, 1.2 eq.) in DMF (0.9 mL) at rt. After 5 min stirring at rt, L6Orn (40.44 mg, 25.7 mmol, 1.0 eq.) and DIEA (20 μL, 114.8 μmol, 4.5 eq.) were added to the reaction mixture. After stirring at rt for 2 h, TFA was added until acidic pH was reached. Purification by preparative HPLC (5 to 100% of ACN+0.1% TFA in water+0.1% TFA) afforded L6Orn-carbonate-PEG₅-NH-Boc (33.10 mg, 13.4 μmol, 90% UV purity, 52% yield) as a white powder after freeze-drying. UPLC-MS (method: 5): Rt=1.30 min, m/z=1058 [M-Boc+2H]²⁺, 706 [M-Boc+3H]³⁺, 1106 [M-2H]²⁻.

Step 4. TFA (1 mL) was added to a solution of L6Orn-carbonate-PEG₅-NH-Boc (33.10 mg, 14.9 μmol, 1.0 eq.) in DCM (2 mL) at rt. The reaction mixture was stirred at rt for 30 min then concentrated in vacuo. Water (7.5 mL) and ACN (7.5 mL) were added and the mixture was freeze-dried to afford L6Orn-carbonate-PEG₅-NH₂ (40.00 mg, 14.2 μmol, 100% UV purity, 95% yield) as a white powder. UPLC-MS (method 5): Rt=1.03, m/z=1824 [M+H]*, 912 [M+2H]²⁺, 1822 [M−H]⁻, 910 [M-2H]²⁻.

### Preparation of PEG₂₀-L6Orn-carbonate-NH₂

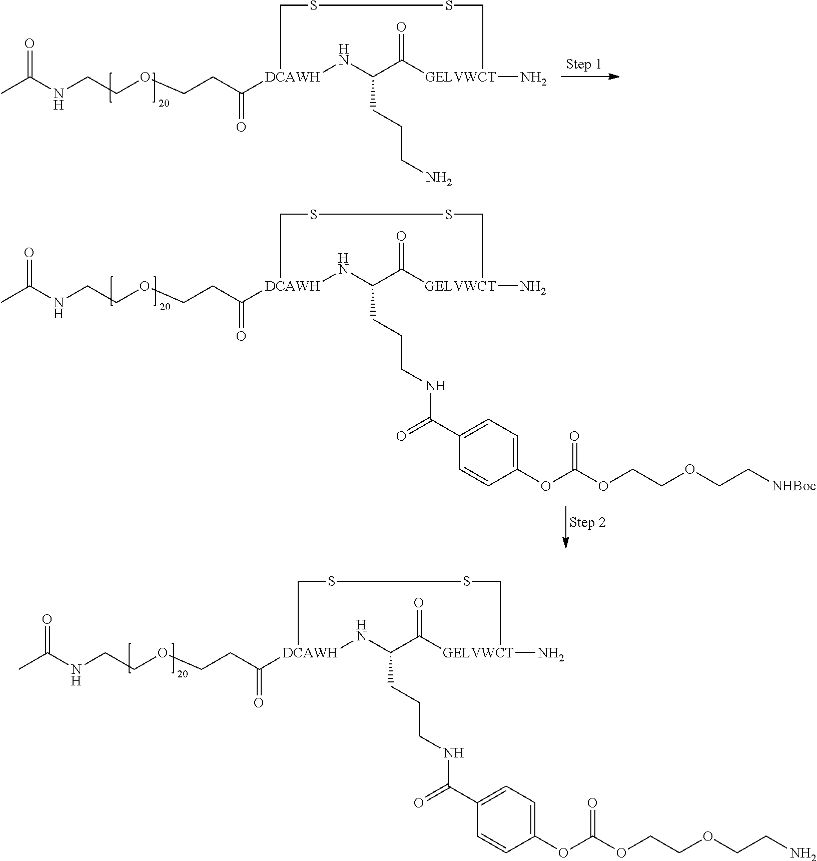

Step 1. HATU (3.97 mg, 10.5 μmol, 1.1 eq.) follower by DIEA (10 μL, 57.0 μmol, 6.0 eq.) were added to a solution of 4-[2-[2-(tert-butoxycarbonateonylamino)ethoxy]ethoxycarbonateonyloxy]benzoic acid (4.21 mg, 11.4 μmol, 1.2 eq.) in DMF (0.8 mL) at rt. After stirring at rt for 5 min, PEG₂₀-L6Orn (24.0 mg, 9.5 μmol, 1.0 eq.) was added to the reaction mixture. After stirring at rt for 10 min, TFA was added until acidic pH was reached. Purification by preparative HPLC (5 to 100% of ACN+0.1% TFA in water+0.1% TFA) afforded PEG₂₀-L6Orn-carbonate-NHBoc (19.2 mg, 6.6 μmol, 99% UV purity, 70% yield) as a white powder after freeze-drying. UPLC-MS (method 4): Rt=2.44 min m/z=1439 [M+2H]²⁺, 1437 [M-2H]²⁻.

Step 2. Trifluoroacetic acid (1 mL) was added to a solution of PEG₂₀-L6Orn-carbonate-NHBoc (19.2 mg, 6.6 μmol, 1.0 eq.) in DCM (1 mL) at rt. After stirring at rt for 30 min, the reaction mixture was concentrated in vacuo. A mixture of ACN/water (10 mL, 1:1) was added and the mixture was freeze-dried to afford PEG₂₀-L6Orn-carbonate-NH₂ (20.1 mg, 6.6 μmol, 100% UV purity, quant.) as a white powder. UPLC-MS (method 4): Rt=1.92 min m/z=1389 [M+2H]²⁺, 1387 [M-2H]²⁻.

### Preparation of PEG₂₄-L6Orn-carbonate-NH₂

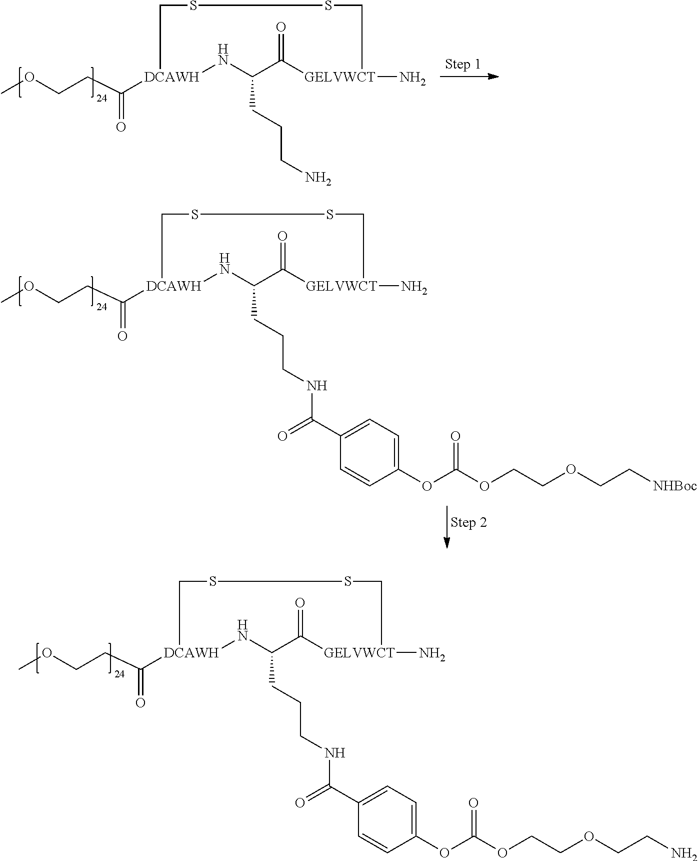

Step 1. 2-(2-aminoethoxy)ethyl (4-fluorocarbonylphenyl) carbonate (14.4 mg, 38.7 μmol, 2.0 eq.) and DIEA (19.0 μL, 38.1 μmol, 2.0 eq.) were added to a solution of PEG₂₄-L6Orn (50.9 mg, 19.0 μmol, 1.0 eq.) in 0.4 mL DMF. After stirring at rt for 4 h, the reaction mixture was precipitated with 9.0 mL of cold ether. UPLC-MS (method: 2): Rt=2.82 min, m/z=1514 [M+2H]²⁺.

Step 2. TFA/TIS/H₂O (570/15/15 μL) was added to a pellet of PEG₂₄-L6Orn-carbonate-NHBoc (57.6 mg, 19.0 μmol, 1.0 eq.) at rt. The reaction mixture was stirred at rt for 1 h, then precipitated with 5.4 mL of cold ether. UPLC-MS (method 2): Rt=2.39, m/z=976 [M+3H]³⁺.

### Preparation of PEG₂₄-L6Orn-carbonate-PEG₃-NH₂

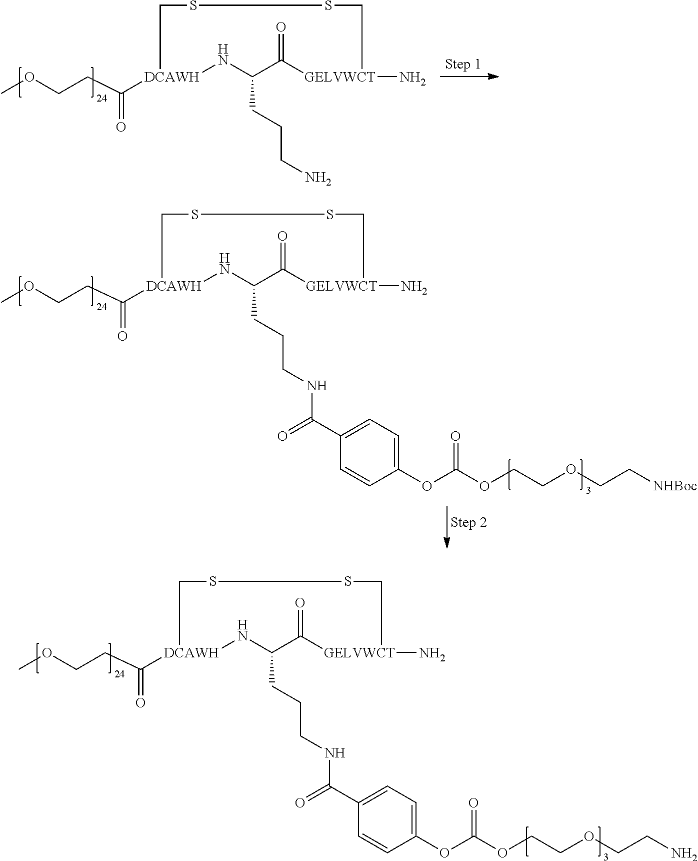

Step 1. HATU (17.49 mg, 46.0 μmol, 1.1 eq.) followed by DIEA (29 μL, 167.3 μmol, 4.0 eq.) were added to a solution of BocHN-PEG₃-carbonate-COOH (28.7 mg, 50.2 μmol, 1.2 eq.) in DMF (4 mL) at rt. After stirring at rt for 5 min, PEG₂₄-L6Orn (110.0 mg, 41.8 μmol, 1.0 eq.) was added to the reaction mixture. After stirring at rt for 10 min, TFA was added until acidic pH was reached. Purification by preparative HPLC (20-60% of ACN+0.1% TFA in water+0.1% TFA) was achieved affording PEG₂₄-L6Orn-carbonate-PEG₃-NHBoc (108.0 mg, 33.8 μmol, 96% UV purity, 81% yield) as a white powder after freeze-drying. UPLC-MS (method 5): Rt=1.42 min, m/z=1533 [M-2H]²⁻.

Step 2. Trifluoroacetic acid (1.6 mL) was added to a solution of PEG₂₄-L6Orn-carbonate-PEG₃-NHBoc (108.0 mg, 33.8 μmol, 1.0 eq.) in DCM (1.6 mL) at rt. After stirring at rt for 15 min, the reaction mixture was concentrated in vacuo. A mixture of ACN/water (12 mL, 1:1) was added then the mixture was freeze-dried to afford PEG₂₄-L6Orn-carbonate-PEG₃-NH₂ (117.2 mg, 33.5 μmol, 93% UV purity, quant.) as a white powder. UPLC-MS (method 5): Rt=1.19 min, m/z=1485 [M+2H]²⁺, 1483 [M-2H]²⁻.

### Preparation of L6Orn(-carbonate-NH₂)-PEG₂₄

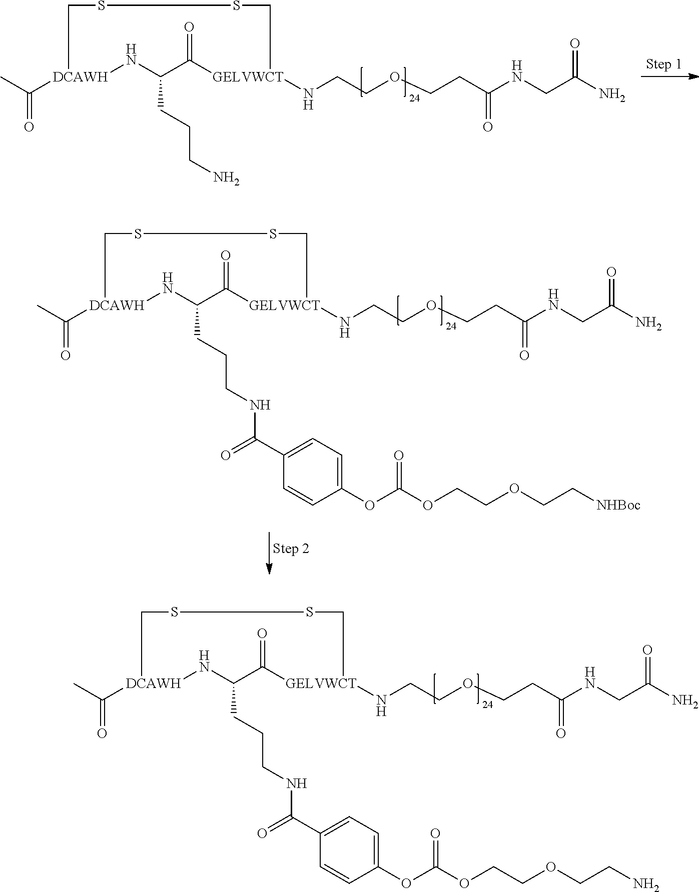

Step 1. HATU (3.79 mg, 10.0 μmol, 1.1 eq.) follower by DIEA (9.5 μL, 54.4 μmol, 6.0 eq.) were added to a solution of 4-[2-[2-(tert-butoxycarbonateonylamino)ethoxy]ethoxycarbonateonyloxy]benzoic acid (4.00 mg, 10.9 μmol, 1.2 eq.) in DMF (0.8 mL) at rt. After stirring at rt for 5 min, L6Orn-PEG₂₄ (25.0 mg, 9.1 μmol, 1.0 eq.) was added to the reaction mixture. After stirring at rt for 10 min, TFA was added until acidic pH was reached. Purification by preparative HPLC (5 to 100% of ACN+0.1% TFA in water+0.1% TFA) afforded L6Orn(-carbonate-NHBoc)-PEG₂₄ (21.8 mg, 6.9 μmol, 100% UV purity, 77% yield) as a white powder after freeze-drying. UPLC-MS (method 4): Rt=2.45 min, m/z=1555 [M+2H]²⁺, 1553 [M-2H]²⁻.

Step 2. Trifluoroacetic acid (1 mL) was added to a solution of L6Orn(-carbonate-NHBoc)-PEG₂₄ (21.8 mg, 6.9 μmol, 1.0 eq.) in DCM (1 mL) at rt. After stirring at rt for 30 min, the reaction mixture was concentrated in vacuo. A mixture of ACN/water (10 mL, 1:1) was added and the mixture was freeze-dried to afford L6Orn(-carbonate-NH₂)-PEG₂₄ (22.0 mg, 6.9 μmol, 100% UV purity, quant.) as a white powder. UPLC-MS (method 4): Rt=1.95 min, m/z=1506 [M+2H]²⁺, 1504 [M-2H]²⁻.

### Preparation of RGNCAYHK(NHS-N₃)GQIIWCTYH

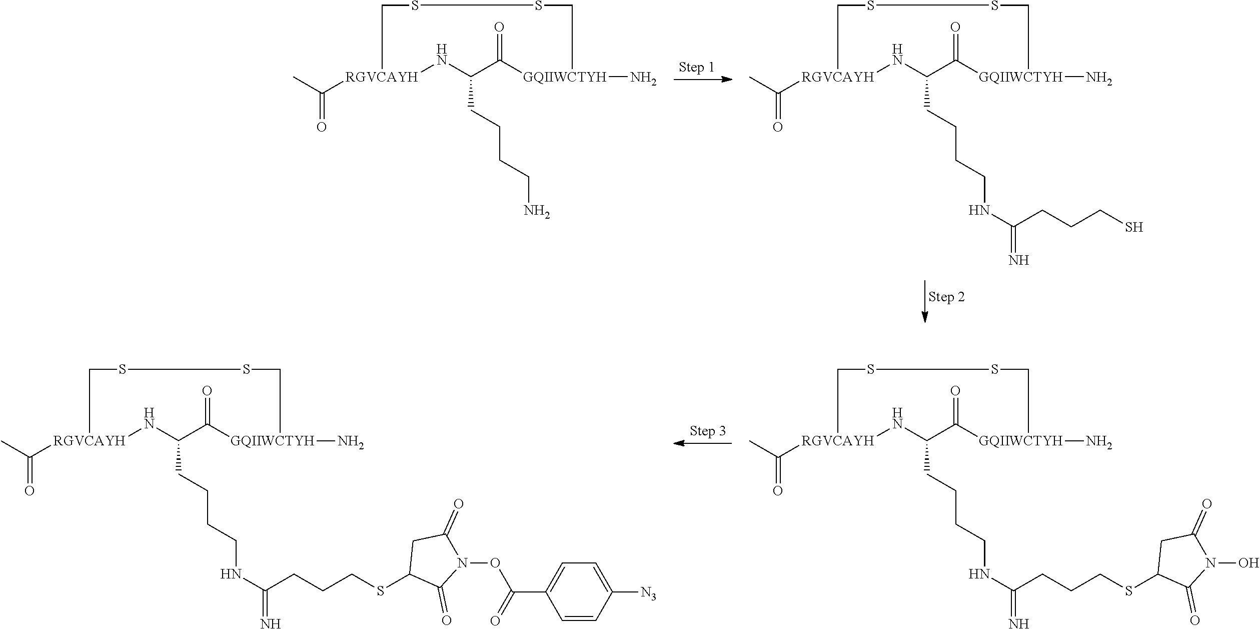

Step 1. DIEA (3.9 μL, 22.3 μmol, 1.9 eq.) was added to a solution of 2-iminothiolane hydrochloride (2.2 mg, 16.0 μmol, 1.4 eq.) followed by addition of RGNCAYHKGQIIWCTYH (24.5 mg, 11.7 μmol, 1.0 eq.) in 0.235 mL DMSO at rt. The reaction was stirring for 2h30 at rt and the product was used without further purification. UPLC-MS (method 2): Rt=1.94 min, m/z=1096.3 [M+2H]²⁺.

Step 2. N-hydroxymaleimide (3.6 mg, 32.0 μmol, 2.8 eq.) was added to a solution of RGNCAYHK(SH)GQIIWCTYH (25.5 mg, 11.7 μmol, 1.0 eq.) in DMSO (0.255 mL) and stirred for 1h at rt. Purification by preparative HPLC (12 to 32% of ACN+0.1% TFA in water+0.1% TFA) afforded RGNCAYHK(SuOH)GQIIWCTYH (11.5 mg, 5.0 μmol, 99% UV purity, 43% yield) as a white solid after freeze-drying. UPLC-MS (method 2): Rt=1.91 min, m/z=1152.8 [M+2H]²⁺.

Step 3. 6-Cl-HOBt (4.4 mg, 3.3 μmol, 1.0 eq) followed by EDCl (5.6 μL, 3.3 μmol, 1.0 eq.) were added to a solution of 4-azidobenzoic acid (0.64 mg, 3.9 μmol, 1.2 eq.) in DMF (20 μL) at rt. After stirring at rt for 1 min, RGNCAYHK(SuOH)GQIIWCTYH (7.5 mg, 3.3 μmol, 1.0 eq.) in DMF (0.16 mL) was added to the reaction mixture. The reaction was stirring at rt for 4h leading to a RGNCAYHK(NHS-N₃)GQIIWCTYH formation, which was used without further purification. UPLC-MS (method 2): Rt=2.25 min, m/z=1225 [M+2H]²⁺.

### Preparation of RGNCAYHK(carbonate-N₃)GQIIWCTYH

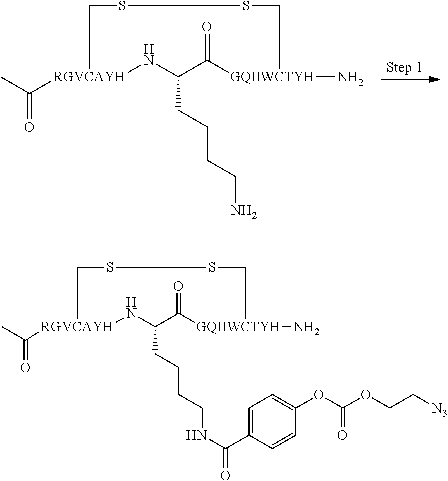

HATU (1.5 mg, 4.0 μmol, 1.4 eq) followed by DIEA (14.4 μL, 28.7 μmol, 10.0 eq.) were added to a solution of compound 018 (1.17 mg, 4.7 μmol, 1.5 eq.) in DMF (21.5 μL) at rt. After stirring at rt for 5 min, RGNCAYHKGQIIWCTYH (6 mg, 2.9 μmol, 1.0 eq.) in DMF (0.3 mL) was added to the reaction mixture. The reaction was stirring at rt for 4h. RGNCAYHK(carbonate-N₃)GQIIWCTYH was used without further purification. UPLC-MS (method 2): Rt=2.25 min, m/z=1162 [M+2H]²⁺.

### Preparation of RGNCAYHOrn(NHS-N₃)GQIIWCTYH

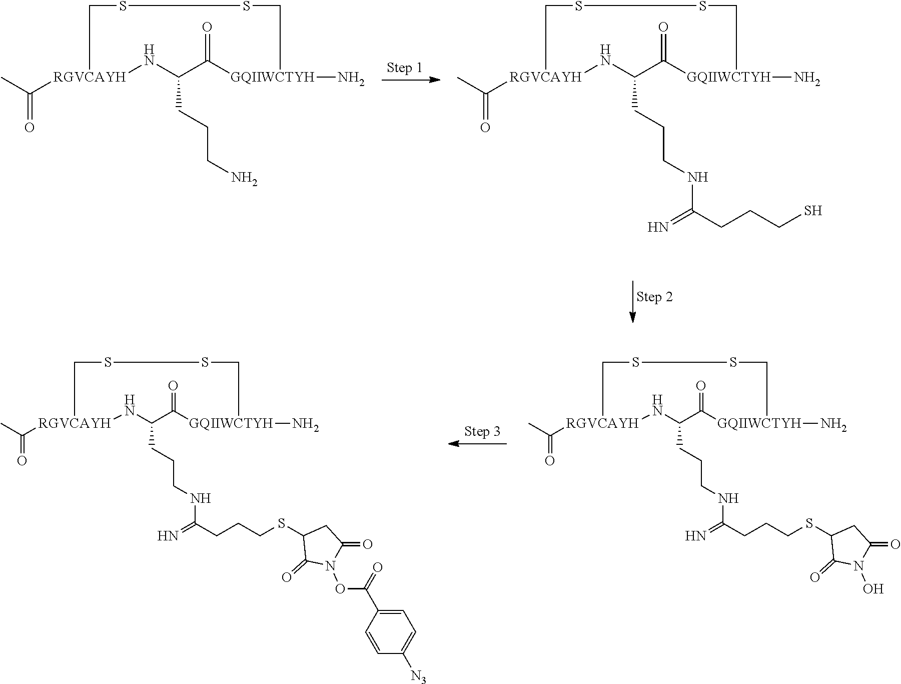

Step 1. DIEA (3.4 μL, 19.6 μmol, 2.3 eq.) was added to a solution of 2-iminothiolane hydrochloride (1.8 mg, 13.1 μmol, 1.5 eq.) followed by addition of RGNCAYHOrnGQIIWCTYH (18.1 mg, 8.7 μmol, 1.0 eq.) in 0.16 mL DMSO at rt. The reaction was stirring for 2h at rt and the product was used without further purification. UPLC-MS (method 2): Rt=1.90 min, m/z=1088.8 [M+2H]²⁺.

Step 2. N-hydroxymaleimide (3.9 mg, 34.2 μmol, 3.9 eq.) was added to a solution of RGNCAYHOrn(SH)GQIIWCTYH (19.1 mg, 8.7 μmol, 1.0 eq.) in DMSO (0.19 mL) and stirred for 1h at rt. Purification by preparative HPLC (16 to 26% of ACN+0.1% TFA in water+0.1% TFA) afforded RGNCAYHOrn(SuOH)GQIIWCTYH (7.1 mg, 3.1 μmol, 99% UV purity, 36% yield) as a white solid after freeze-drying. UPLC-MS (method 2): Rt=1.87 min, m/z=1145.4 [M+2H]²⁺.

Step 3. 6-Cl-HOBt (0.3 mg, 2.1 μmol, 1.0 eq) followed by EDCl (0.4 μL, 2.1 μmol, 1.0 eq.) were added to a solution of 4-azidobenzoic acid (0.4 mg, 2.5 μmol, 1.2 eq.) in DMF (13 μL) at rt. After stirring at rt for 1 min, RGNCAYHOrn(SuOH)GQIIWCTYH (4.8 mg, 2.1 μmol, 1.0 eq.) in DMF (0.1 mL) was added to the reaction mixture. The reaction was stirring at rt for 4h leading to a RGNCAYHOrn(NHS-N₃)GQIIWCTYH formation, which was used without further purification. UPLC-MS (method 2): Rt=2.25 min, m/z=1218.9 [M+2H]²⁺.

### Preparation of RGNCAYHK(carbonate-NH₂)GQIIWCTYH

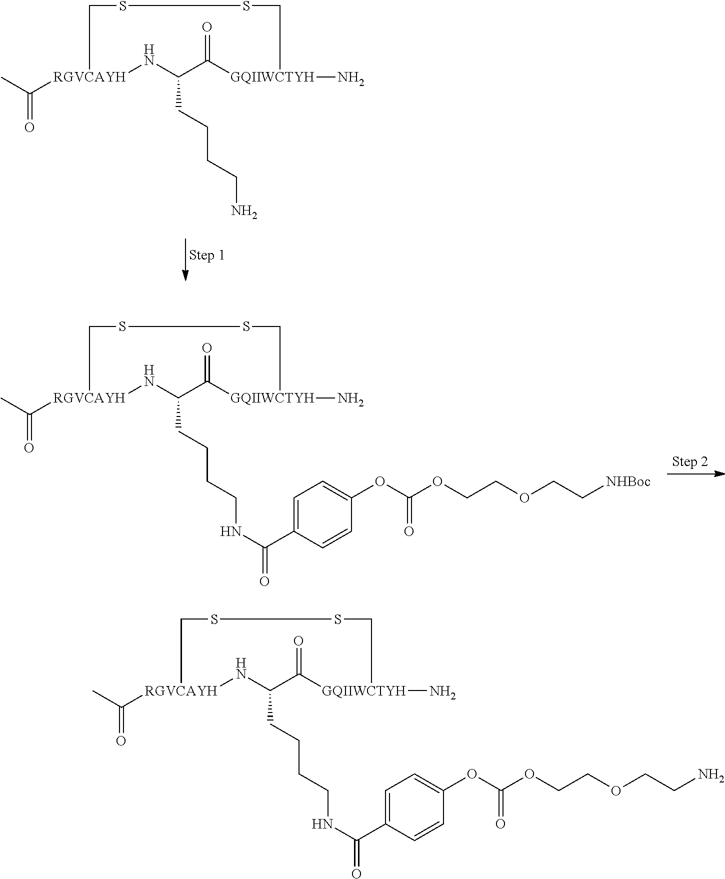

Step 1. 2-(2-aminoethoxy)ethyl (4-fluorocarbonylphenyl) carbonate (5.1 mg, 13.7 μmol, 1.9 eq.) and DIEA (12.7 μL, 25.4 μmol, 3.5 eq.) were added to a solution of RGNCAYHKGQIIWCTYH (17.5 mg, 7.2 μmol, 1.0 eq.). After stirring at rt for 5 h, the reaction mixture was precipitated with 4.7 mL of cold ether. UPLC-MS (method: 2): Rt=2.29 min, m/z=1221.2 [M+2H]²⁺.

Step 2. TFA/TIS/H₂O (190/5/5 μL) was added to a pellet of RGNCAYHK(carbonate-NHBoc)GQIIWCTYH (33.10 mg, 7.2 μmol, 1.0 eq.) at rt. The reaction mixture was stirred at rt for 1 h then precipitated with 1.8 mL of cold ether. UPLC-MS (method 2): Rt=1.90, m/z=1170.8 [M+2H]²⁺.

### Preparation of L6Orn-carbonate-DTPA

p-SCN-Bn-CHX-A″-DTPA.3HCl (19.1 mg, 27.2 μmol, 1.0 eq) was added to a solution of L6Orn-carbonate-NH₂ (49.6 mg, 27.2 μmol, 1.0 eq.) and TEA (22 μL, 160 mmol, 6.0 eq.) in DMF (0.4 mL) at rt. After stirring at rt for 3 h, TFA was added until acidic pH was reached. Purification by reversed phase automated flash chromatography on a C18 biotage column (30 g, 25 to 60% ACN+0.1% TFA in H₂O+0.1% TFA over 12 CV) afforded L6Orn-carbonate-DTPA (45.2 mg, 10.6 μmol, 90% UV purity, 62% yield). UPLC-MS (method 4): Rt=2.11 min, m/z=1210 [M+2H]²⁺, 1208 [M-2H]²⁻.

### Preparation of L6Orn-carbonate-PCTA

DIEA (1.9 μL, 10.8 μmol, 4.0 eq.) was added to a solution of p-SCN-Bn-PCTA.3HCl (1.75 mg, 2.7 μmol, 1.0 eq.) and L6Orn-carbonate-NH₂ (5.0 mg, 2.7 μmol, 1.0 eq.) in DMF (0.3 mL) at rt. After stirring at rt for 1.5 h, TFA (4 μL) was added to the reaction mixture. Purification by preparative HPLC on C18 (24 to 36% ACN+0.1% TFA in water+0.1% TFA) afforded L6Orn-carbonate-PCTA (2.26 mg, 0.96 μmol, 96% UV purity, 34% yield). UPLC-MS (method 2): Rt=2.46 min, m/z=1176.4 [M+2H]²⁺.

### Preparation of L6Orn-carbonate-NODA-GA

DIEA (1.9 μL, 10.8 μmol, 4.0 eq.) was added to a solution of NODA-GA-NHS ester·TFA·HPF₆ (1.97 mg, 2.7 μmol, 1.0 eq.) and L6Orn-carbonate-NH₂ (5.0 mg, 2.7 μmol, 1.0 eq.) in DMF (0.1 mL) at rt. After stirring at rt for 3.5 h, NODA-GA-NHS ester·TFA·HPF₆ (1.97 mg, 2.7 μmol, 1.0 eq.) was added to the reaction mixture at rt. After stirring at rt for 1 h, TFA (4 μL) was added to the reaction mixture. Purification by preparative HPLC on C18 (21 to 33% ACN+0.1% TFA in water+0.1% TFA) afforded L6Orn-carbonate-NODA-GA (1.44 mg, 0.66 μmol, 87% UV purity, 21% yield). UPLC-MS (method 2): Rt=2.303 min, m/z=1091.4 [M+2H]²⁺.

### Preparation of L6Orn-carbonate-Bn-NOTA

DIEA (1.9 μL, 11.0 μmol, 4.0 eq.) was added to a solution of p-SCN-Bn-NOTA (1.53 mg, 2.7 μmol, 1.0 eq.) and L6Orn-carbonate-NH₂ (5.0 mg, 2.7 μmol, 1.0 eq.) in DMF (0.3 mL) at rt. After stirring at rt for 1.5 h, TFA was added until acidic pH was reached. Purification on C18 (12 g, 20 to 80% of ACN+0.1% TFA in water+0.1% TFA over 12 CV) afforded L6Orn-carbonate-NOTA (4.2 mg, 1.8 μmol, UV purity 100%, 67% yield) as a white powder after freeze-drying. UPLC-MS (method 4): Rt=2.12 min, m/z=1138 [M+2H]²⁺, 1136 [M-2H]²⁻.

### Preparation of L6Orn-carbonate-DOTA-GA

DIEA (1.9 μL, 11.0 μmol, 4.0 eq.) was added to a solution of DOTA-GA(OtBu)₄ (2.1 mg, 3.0 μmol, 1.1 eq.) and HATU·HPF₆ (1.15 mg, 3.0 μmol, 1.1 eq.) in DMF (0.3 mL) at rt. After stirring at rt for 5 min, the reaction mixture was added to L6Orn-carbonate-NH₂ (5.0 mg, 2.7 μmol, 1.0 eq.) at rt. After stirring at rt for 20 min, TFA was added until acidic pH was reached. Purification on C18 (12 g, 20 to 80% of ACN+0.1% TFA in water+0.1% TFA over 12 CV) afforded L6Orn-carbonate-DOTA-GA(OtBu)₄ (4.1 mg, 1.6 μmol, UV purity 99%, 59% yield) as a white powder after freeze-drying. The latter compound was dissolved in DCM (0.5 mL) and TFA (0.5 mL) and stirred at rt. After stirring at rt for 21 h, the reaction mixture was concentrated in vacuo then purification on C18 (12 g, 20 to 50% of ACN+0.1% TFA in water+0.1% TFA over 10 CV) afforded L6Orn-carbonate-DOTA-GA (0.7 mg, 0.2 μmol, UV purity 74%, 14% yield) as a white powder after freeze-drying. UPLC-MS (method 4): Rt=1.85 min, m/z=761 [M+3H]³⁺, 1140 [M-2H]²⁻.

### Preparation of L6Orn-carbonate-DFO

To a solution of L6Orn-carbonate-NH₂ (8.0 mg, 4.4 μmol, 1.0 eq.) in DMF (0.1 mL) at rt was added DFO-SCN (3.3 mg, 4.4 μmol, 1.0 eq.) followed by DIEA (6.1 μL, 35.1 μmol, 8.0 eq.). After stirring for 2.5 h at rt, DIEA (3.0 μL, 17.5 μmol, 4.0 eq.) was added. After stirring for 45 min at rt, 5 drops of a 0.1% TFA in water solution were added and the reaction mixture was stirred at rt for 5 min. Purification by preparative HPLC (5 to 100% ACN+0.1% FA in water+0.1% FA) was afforded L6Orn-carbonate-DFO (2.0 mg, 0.6 μmol, 80% UV purity, 14% yield) as a white powder after freeze-drying. UPLC-MS (method 4): Rt=2.27 min, m/z=860 [M+3H]³⁺, 1287 [M-2H]²⁻.

### Preparation of L6Orn-carbonate-triazole-dbco-DFO

DBCO-DFO (2.13 mg, 2.5 μmol, 1.0 eq.) was added to a mixture of L6Orn-carbonate-N₃ (4.8 mg, 2.5 μmol, 1.0 eq.) in DMF (0.3 mL) at rt. After stirring at rt for 50 min, more of L6Orn-carbonate-N₃ (0.5 mg, 0.1 eq.) was added to the reaction mixture. After stirring at rt for 1.5 h, L6Orn-carbonate-N₃ (0.5 mg, 0.1 eq.) was added to the reaction mixture. After stirring at rt for 3.5 h, TFA was added until acidic pH was reached. Purification on C18 (12 g, 20 to 80% of ACN+0.1% TFA in water+0.1% TFA over 12 CV) afforded L6Orn-carbonate-triazole-dbco-DFO (0.9 mg, 0.3 μmol, UV purity 84%, 11% yield) as a white powder after freeze-drying. UPLC-MS (method 4): Rt=2.34 min, m/z=919 [M+3H]³⁺, 1376 [M-2H]²⁻.

### Preparation of L6Orn-carbonate-S-maleimide-DFO

A degassed mixture of TFA (4 μL) and DCM (196 μL) was added to a solution of L6Orn-carbonate-S-Mmt (3.0 mg, 1.4 μmol, 1.0 eq.). The reaction mixture was stirred at rt for 5 min then added to ma-DFO (1.46 mg, 2.1 μmol, 1.5 eq.) followed by DIEA (3.6 μL, 20.6 μmol, 15 eq.). After stirring at rt for 1 h, TFA was added until acidic pH was reached. Purification by reversed phase preparative HPLC (5−50% ACN+0.1% FA in water+0.1% FA) afforded L6Orn-carbonate-S-maleimide-DFO (1.3 mg, 0.5 μmol, UV purity 98%, 35% yield) as a white powder after freeze-drying. UPLC-MS (method 4): Rt=2.11 min, m/z=1313 [M+2H]²⁺, 1311 [M-2H]²⁻.

### Preparation of L6Orn-carbonate-FAM

HATU·HPF₆ (5.58 μL, 3.02 μmol, 1.1 eq.) was added to a solution of 5(6)-carboxyfluorescein (FAM) (1.24 mg, 3.3 μmol, 1.2 eq.) and stirred for 1 min, followed by the addition of DIEA (1.64 μL, 3.3 μmol, 1.2 eq.) at rt. After 3 min stirring at rt, L6Orn-carbonate-NH₂ (5.0 mg, 2.7 μmol, 1.0 eq.) in DMF (50 μL) was added to the reaction mixture and stirred at rt for 2 h. Completion of the reaction was monitored by UPLC-MS. Purification by preparative HPLC on C18 (26 to 38% ACN+0.1% TFA in water+0.1% TFA) was afforded L6Orn-carbonate-FAM (0.5 mg, 0.23 μmol, 96% UV purity, 8% yield) as a yellow powder after freeze-drying. UPLC-MS (method 1): Rt=1.9 min, m/z=1091.61 [M+2H]²⁺.

### Preparation of L6Orn-carbonate-TZ

DIEA (1.9 μL, 11.0 μmol, 4.0 eq.) was added to a solution of L6Orn-carbonate-NH₂ (5.0 mg, 2.7 μmol, 1.0 eq.) and Tz-PEG₅-NHS ester (1.66 mg, 2.7 μmol, 1.0 eq.) in DMF (0.1 mL) at rt. After stirring at rt for 2 h, more of Tz-PEG₅-NHS ester (1.66 mg, 2.7 μmol, 1.0 eq.) in DMF (50 μL) was added to the reaction mixture. After stirring at rt for 3h, TFA was added until acidic pH was reached. Purification by reversed phase automated flash chromatography on a C18 biotage column (12 g, 20 to 80% of ACN+0.1% TFA in water+0.1% TFA over 12 CV) afforded L6Orn-carbonate-Tz (6.2 mg, 2.4 μmol, UV purity 91%, 89% yield) as a pale pink powder after freeze-drying. UPLC-MS (method 4): Rt=2.29 min, m/z=1157 [M+2H]²⁺, 1155 [M-2H]²⁻.

### Preparation of L6Orn-carbonate-TCO

DIEA (1.9 μL, 10.8 μmol, 4.0 eq.) was added to a solution of TCO-NHS ester (0.72 mg, 2.7 μmol, 1.0 eq.) and L6Orn-carbonate-NH₂ (5.0 mg, 2.7 μmol, 1.0 eq.) in DMF (0.1 mL) at rt. After stirring at rt for 1.5 h, TFA (4 μL) was added to the reaction mixture. Purification on C18 (12 g, 10 to 50% of ACN+0.1% TFA in water+0.1% TFA over 12 CV) afforded L6Orn-carbonate-TCO (2.8 mg, 1.10 μmol, UV purity 80%, 41% yield) as a white powder after freeze-drying. UPLC-MS (method 4): Rt=2.75 min, m/z=988 [M+2H]²⁺, 986 [M-2H]²⁻.

### Preparation of L6Orn-carbonate-N₃

DIEA (1.9 μL, 10.8 μmol, 4.0 eq.) was added to a solution of (2,5-dioxopyrrolidin-1-yl) 2-azidoacetate (0.53 mg, 2.7 μmol, 1.0 eq.) and L6Orn-carbonate-NH₂ (5.0 mg, 2.7 μmol, 1.0 eq.) in DMF (0.1 mL) at rt. After stirring at rt for 3 h, TFA (4 μL) was added to the reaction mixture. Purification by reversed phase automated flash chromatography on a C18 biotage column (12 g, 10 to 50% of ACN+0.1% TFA in water+0.1% TFA over 12 CV) afforded L6Orn-carbonate-N₃ (2.7 mg, 1.3 μmol, UV purity 94%, 49% yield) as a white powder after freeze-drying. UPLC-MS (method 4): Rt=2.16 min, m/z=954 [M+2H]²⁺, 952 [M-2H]²⁻.

### Preparation of L6Orn-carbonate-DBCO

DIEA (1.9 μL, 10.8 μmol, 4.0 eq.) was added to a solution of DBCO-NHS ester (1.12 mg, 2.7 μmol, 1.0 eq.) and L6Orn-carbonate-NH₂ (5.0 mg, 2.7 μmol, 1.0 eq.) in DMF (0.1 mL) at rt. After stirring at rt for 1.5 h, TFA (4 μL) was added to the reaction mixture. Purification on C18 (32 to 44% of ACN+0.1% TFA in water+0.1% TFA) afforded L6Orn-carbonate-DBCO (2.27 mg, 1.07 μmol, UV purity 97%, 38% yield) as a white powder after freeze-drying. UPLC-MS (method 4): Rt=2.79 min, m/z=1056 [M+2H]²⁺, 1055 [M-2H]²⁻.

### Preparation of L6Orn-carbonate-BCN

DIEA (1.9 μL, 10.8 μmol, 4.0 eq.) was added to a solution of BCN—NHS ester (0.83 mg, 2.7 μmol, 1.0 eq.) and L6Orn-carbonate-NH₂ (5.0 mg, 2.7 μmol, 1.0 eq.) in DMF (0.1 mL) at rt. After stirring at rt for 2.5 h, TFA (4 μL) was added to the reaction mixture. Purification on C18 (34 to 46% of ACN+0.1% TFA in water+0.1% TFA) afforded L6Orn-carbonate-BCN (2.03 mg, 1.01 μmol, UV purity 95%, 35% yield) as a white powder after freeze-drying. UPLC-MS (method 2): Rt=2.903 min, m/z=1000.8 [M+2H]²⁺.

### Preparation of L6Orn-carbonate-maleimide

DIEA (1.9 μL, 10.8 μmol, 4.0 eq.) was added to a solution of 2,5-dioxopyrrolidin-1-yl 3-(2-(2-(3-(2,5-dioxo-2h-pyrrol-1(5h)-yl)propanamido)ethoxy)ethoxy)propanoate (1.2 mg, 2.7 μmol, 1.0 eq.) and L6Orn-carbonate-NH₂ (5.0 mg, 2.7 μmol, 1.0 eq.) in DMF (0.1 mL) at rt. After stirring at rt for 2 h, more of 2,5-dioxopyrrolidin-1-yl 3-(2-(2-(3-(2,5-dioxo-2h-pyrrol-1(5h)-yl)propanamido)ethoxy)ethoxy)propanoate (1.2 mg, 2.7 μmol, 1.0 eq.) in DMF (50 μL) was added to the reaction mixture. After stirring at rt for 3 h, TFA (4 mL) was added to the reaction mixture. Purification on C18 (12 g, 10 to 50% of ACN+0.1% TFA in water+0.1% TFA over 12 CV) afforded L6Orn-carbonate-maleimide (1.6 mg, 0.7 μmol, UV purity 91%, 25% yield) as a white powder after freeze-drying. UPLC-MS (method 4): Rt=2.07 min, m/z=1067 [M+2H]²⁺, 1065 [M-2H]²⁻.

### Preparation of L6Orn-carbonate-MCC-DM1

DIEA (1.9 μL, 10.8 μmol, 4.0 eq.) was added to a solution of DM1-SMCC (2.79 mg, 2.7 μmol, 1.0 eq.) and L6Orn-carbonate-NH₂ (5.0 mg, 2.7 μmol, 1.0 eq.) in DMF (0.2 mL) at rt. After stirring at rt for 70 min, TFA (4 μL) was added to the reaction mixture.

Purification by reversed phase automated flash chromatography on a C18 biotage column (12 g, 20 to 80% of ACN+0.1% TFA in water+0.1% TFA over 12 CV) afforded L6Orn-carbonate-MCC-DM1 (5.23 mg, 1.80 μmol, UV purity 96%, 69% yield) as a white powder after freeze-drying. UPLC-MS (method 4): Rt=2.80 min, 1390 [M-2H]²⁻.

### Preparation of L6Orn-carbonate-SPDB-DM4

A saturated aqueous NaHCO₃ solution (83 μL) was added to a mixture of DM4 (4.28 mg, 5.5 μmol, 1.0 eq.) and SPDB (0.89 mg, 2.7 μmol, 0.5 eq.) in DMA (0.75 mL) at rt. After stirring at rt for 110 min, SPDB (0.89 mg, 2.7 μmol, 0.5 eq.) in DMA (0.2 mL) was added to the reaction mixture at rt. After stirring at rt for 10 min, the reaction mixture was added to L6Orn-carbonate-NH₂ (10.0 mg, 5.5 μmol, 1.0 eq.) at rt. After stirring at rt for 1 h, TFA was added until acidic pH was reached. Purification on C18 (30 g, 20 to 80% of ACN+0.1% TFA in water+0.1% TFA over 12 CV) afforded L6Orn-carbonate-SPDB-DM4 (10.7 mg, 3.9 μmol, UV purity 99%, 71% yield) as a white product after freeze-drying. UPLC-MS (method 4): Rt=2.99 min, m/z=1351 [M-2H]²⁻.

### Preparation of L6Orn-carbonate-diaminoxylene(-SPDB-DM4)₂

A saturated aqueous NaHCO₃ solution (83 μL) was added to a mixture of DM4 (3.93 mg, 5.0 μmol, 2.0 eq.) and SPDB (1.64 mg, 5.0 μmol, 2.0 eq.) in DMA (0.75 mL) at rt. After stirring at rt for 5 min, the reaction mixture was added to L6Orn-carbonate-diaminoxylene (5.0 mg, peptide content 50%, 1.25 μmol, 0.5 eq.) at rt. After stirring at rt for 2 h, more of L6Orn-carbonate-diaminoxylene (5.0 mg, peptide content 50%, 1.25 mmol, 0.5 eq.) was added to the reaction mixture. After stirring at rt for 40 min, TFA was added until acidic pH was reached. Purification on C18 (30 g, 20 to 80% of ACN+0.1% TFA in water+0.1% TFA over 12 CV) afforded L6Orn-carbonate-(SPDB-DM4)₂ (2.0 mg, 0.5 mmol, UV purity 87%, 18% yield) as a white product after freeze-drying. UPLC-MS (method 4): Rt=3.49 min, m/z=1873 [M-2H]²⁻.

### Preparation of L6Orn-carbonate-Vedotin

A degassed mixture of TFA (20 μL) and DCM (80 μL) was added to a solution of L6Orn-carbonate-S-Mmt (8.0 mg, 3.6 μmol, 1.2 eq.). The reaction mixture was stirred at rt for 5 min then added to Vedotin (4.0 mg, 3.0 μmol, 1.0 eq.) followed by DIEA (37 μL, 0.21 μmol, 70 eq.). After stirring at rt for 20 min, TFA was added until acidic pH was reached. Purification by reversed phase automated flash chromatography on a C18 biotage column (12 g, 20−80% ACN+0.1% TFA in water+0.1% TFA over 13 CV) gave L6Orn-carbonate-Vedotin (6.0 mg, 1.7 μmol, UV purity 94%, 57% yield) as a white powder after freeze-drying. UPLC-MS (method 4): Rt=3.01 min, m/z=1077 [M+3H]³⁺, 1613 [M-2H]²⁻.

### Preparation of L6Orn-carbonate-(Vedotin)₂

HATU·HPF₆ (0.61 mg, 1.6 μmol, 1.1 eq.) and DIEA (1.0 mL, 5.9 μmol, 4.0 eq.) were added to a solution of dihydroasparagusic acid-(Vedotin)₂ (5.1 mg, 1.8 μmol, 1.2 eq.) in DMF (0.2 mL) at rt. After stirring at rt for 5 min, the reaction mixture was added to L6Orn-carbonate-NH₂ (4.46 mg, 1.5 μmol, 1.0 eq.). After stirring at rt for 30 min, TFA was added until acidic pH was reached. Purification by reversed phase automated flash chromatography on a C18 biotage column (12 g, 20 to 80% of ACN+0.1% TFA in water+0.1% TFA over 12 CV) afforded L6Orn-carbonate-(Vedotin)₂ (2.69 mg, 0.6 μmol, UV purity 97%, 39% yield) as a white lyophilizate. UPLC-MS (method 4): Rt=3.49 min, m/z=1148 [M+4H]⁴⁺, 1531 [M+3H]³⁺, 1529 [M-3H]³⁻.

### Preparation of L6Orn-carbonate-multi-DM1

A degassed mixture of DCM (245 μL) and TFA (5 μL) was added to a solution of L6Orn-carbonate-S-Mmt (2.35 mg, 1.1 μmol, 1.2 eq.). The reaction mixture was stirred at rt for 5 min, then compound 012 (1.5 mg, 0.9 μmol, 1.0 eq.) was added followed by DIEA (11 μL, 62.8 μmol, 70 eq.). After stirring at rt for 10 min, TFA was added until acidic pH was reached. Purification by preparative HPLC (5−100% ACN+0.1% FA in water+0.1% FA) was achieved yielding L6Orn-carbonate-multi-DM1 (0.7 mg, 0.2 μmol, UV purity 98%, 22% yield) as a white powder after freeze-drying. UPLC-MS (method 4): Rt=2.06 min, m/z=1792 [M-2H]²⁻.

### Preparation of L6Orn-carbonate-multi-(DM1)₂

A degassed mixture of TFA (10 μL) and DCM (0.49 mL) was added to a solution of L6Orn-carbonate-S-Mmt (1.25 mg, 0.6 μmol, 1.2 eq.). The reaction mixture was stirred at rt for 5 min, then compound 016 (2.45 mg, 0.5 μmol, 1.0 eq.) in solution in DMF (0.3 mL) was added followed by DIEA (14.7 μL, 0.08 mmol, 176 eq.). After stirring at rt for 10 min, TFA was added until acidic pH was reached. Purification by reversed phase preparative HPLC (5-50% ACN+0.1% FA in water+0.1% FA) gave L6Orn-carbonate-multi-(DM1)₂ (0.8 mg, 0.1 μmol, UV purity 97%, 26% yield) as white lyophilizate. UPLC-MS (method 4): Rt=2.59 min, m/z=1568.8 [M+4H]⁴⁺.

### Preparation of L6Orn-carbonate-PEGs-Bn-NOTA

DIEA (8.0 μL, 50.0 μmol, 10.0 eq.) was added to a solution of p-SCN-Bn-NOTA (3.03 mg, 5.4 μmol, 1.2 eq.) and L6Orn-carbonate-PEG₅-NH₂ (9.80 mg, 4.6 μmol, 1.0 eq.) in DMF (0.4 mL). After stirring at rt for 1.5 h, TFA was added until acidic pH was reached. Purification by preparative HPLC (5 to 100% of ACN+0.1% TFA in water+0.1% TFA) afforded L6Orn-carbonate-PEG₅-Bn-NOTA (1.83 mg, 0.6 μmol, 86% UV purity, 13% yield) as a white powder after freeze-drying. UPLC-MS (method 5): Rt=1.12 min, m/z=856 [M+3H]³⁺, 1282 [M-2H]²⁻.

### Preparation of A3hK(-carbonate-DOTA)-E8Orn(-carbonate-DOTA)

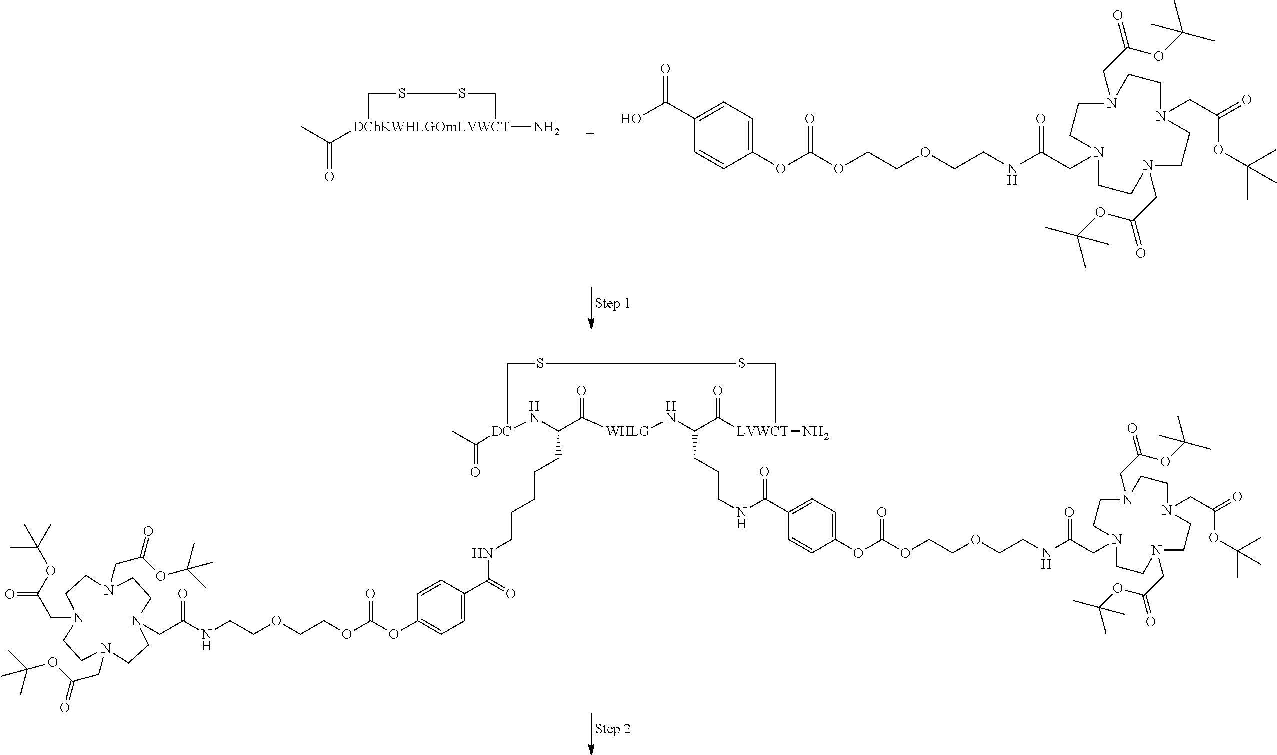

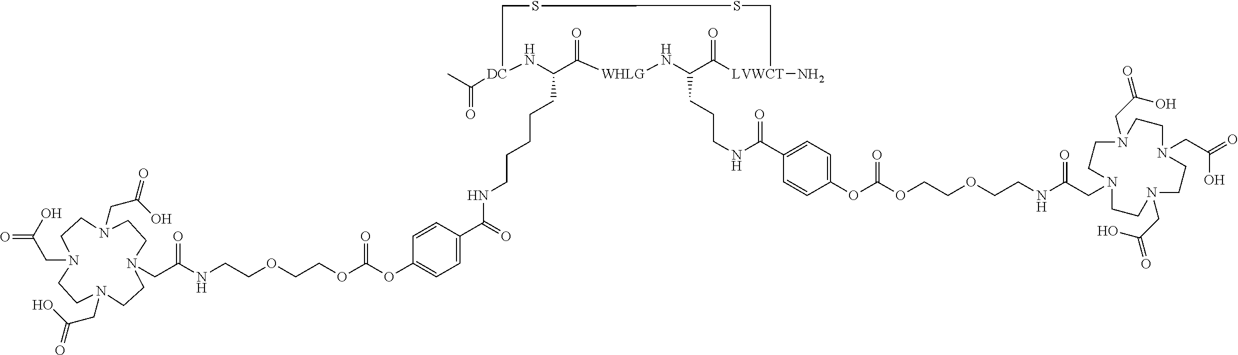

Step 1. HATU (2.57 mg, 6.8 μmol, 2.2 eq.) followed by DIEA (3.2 μL, 18.4 μmol, 6.0 eq.) were added to a solution of compound 006 (6.07 mg, 7.4 μmol, 2.4 eq.) in DMF (0.8 mL) at rt. After stirring at rt for 5 min, A3hK-E8Orn (5.00 mg, 3.1 μmol, 1.0 eq.) was added to the reaction mixture. After stirring at rt for 2.5 h, TFA was added until acidic pH was reached. Purification by preparative HPLC (10 to 100% of ACN+0.1% TFA in water+0.1% TFA) afforded A3hK(-carbonate-DOTA(OtBu)₃)-E8Orn(-carbonate-DOTA(OtBu)₃) (6.10 mg, 1.7 μmol, 90% UV purity, 55% yield) as a white powder after freeze-drying. UPLC-MS (method 4): Rt=3.02 min, m/z=1080 [M+3H]³⁺, 1619 [M-2H]²⁻.

Step 2. TFA was added to a solution of A3hK(-carbonate-DOTA(OtBu)₃)-E8Orn(-carbonate-DOTA(OtBu)₃) (6.1 mg, 1.9 μmol, 1.0 eq.) in at rt. After stirring at rt for 10 min, the reaction mixture was concentrated in vacuo. Purification by preparative HPLC (10 to 60% of ACN+0.1% TFA in water+0.1% TFA) afforded A3hK(-carbonate-DOTA)-E8Orn(-carbonate-DOTA) (3.70 mg, 1.3 μmol, 100% UV purity, 68% yield) as a white powder after freeze-drying. UPLC-MS (method 4): Rt=1.86 min, m/z=1452 [M+2H]²⁺, 1450 [M-2H]²⁻.

### Preparation of L6Orn-carbonate-Bn-NODAGA

DIEA (16.0 μL, 91.9 μmol, 8.4 eq.) was added to a solution of L6Orn-carbonate-NH₂ (20.00 mg, 11.0 μmol, 1.0 eq.) and p-SCN-Bn-NODA-GA (6.00 mg, 11.5 μmol, 1.0 eq.) in DMF (0.4 mL). After stirring at rt for 3 h, TFA was added until acidic pH was reached. Purification by preparative HPLC (5 to 100% of ACN+0.1% TFA in water+0.1% TFA) afforded L6Orn-carbonate-Bn-NODA-GA (17.16 mg, 7.3 μmol, 100% UV purity, 25% yield) as a white powder after freeze-drying. UPLC-MS (method 5): Rt=1.08 min, m/z=1173 [M+2H]²⁺, 782 [M+3H]³⁺, 1172 [M-2H]²⁻.

### Preparation of L6Orn-carbonate-Bn-TCMC

DIEA (30.12 μL, 60.2 μmol, 11.0 eq.) was added to a solution of L6Orn-carbonate-NH₂ (10.00 mg, 5.48 μmol, 1.0 eq.) and p-SCN-Bn-TCMC (5.70 mg, 8.22 μmol, 1.5 eq.) in DMF (0.13 mL). After stirring at rt for 1 h, HCl was added until acidic pH was reached. Purification by preparative HPLC (18 to 32% of ACN+0.1% TFA in water+0.1% TFA) afforded L6Orn-carbonate-Bn-TCMC (3.91 mg, 1.5 μmol, 98% UV purity, 28% yield) as a white powder after freeze-drying. UPLC-MS (method 2): Rt=2.17 min, m/z=1186 [M+2H]²⁺, 791.5 [M+3H]³⁺.

### Preparation of A3hK(-carbonate-N₃)-E8Orn(-carbonate-N₃)

HATU (4.50 mg, 11.8 μmol, 2.8 eq.) followed by DIEA (4.5 μL, 25.8 μmol, 6.0 eq.) were added to a solution of 4-[2-[2-[(2-azidoacetyl)amino]ethoxy]ethoxycarbonateonyloxy]benzoic acid (3.60 mg, 10.2 μmol, 2.4 eq.) in DMF (0.6 mL) at rt. After stirring at rt for 5 min, A3hK-E8Orn (7.00 mg, 4.3 μmol, 1.0 eq.) was added to the reaction mixture. After stirring at rt for 2.5 h, TFA was added until acidic pH was reached. Purification by preparative HPLC (5 to 50% of ACN+0.1% TFA in water+0.1% TFA) afforded A3hK(-carbonate-N₃)-E8Orn(-carbonate-N₃) (4.30 mg, 1.9 μmol, 100% UV purity, 44% yield) as a white powder after freeze-drying. UPLC-MS (method 4): Rt=2.43 min, m/z=1150 [M+2H]²+1147 [M-2H]²⁻.

### Preparation of L6Orn-carbonate-mPEG₃₇

DIEA (23.0 μL, 131.4 μmol, 8.0 eq.) was added to a solution of m-PEG₃₇-NHS ester (29.16 mg, 16.3 μmol, 1.0 eq.) and L6Orn-carbonate-NH₂ (30.06 mg, 16.5 μmol, 1.0 eq.) in DMF (0.6 mL). After stirring at rt for 2.5 h, TFA was added until acidic pH was reached. Purification by preparative HPLC (5 to 100% of ACN+0.1% TFA in water+0.1% TFA) afforded L6Orn-carbonate-mPEG₃₇ (20.80 mg, 5.4 μmol, 90% UV purity, 33% yield) as a white powder after freeze-drying. UPLC-MS (method 5): Rt=1.31 min, m/z=1746 [M-2H]²⁻.

### Preparation of L6Orn-diamino-(mPEG₃₇)₂

DIEA (6.4 μL, 36.8 μmol, 5.0 eq.) was added to a solution of mPEG₃₇-NHS ester (29.80 mg, 16.7 μmol, 2.3 eq.) and L6Orn-carbonate-diaminoxylene (14.61 mg, 7.4 μmol, 1.0 eq.) in DMF (0.5 mL). After stirring at rt for 1 h, TFA was added until acidic pH was reached. Purification by preparative HPLC (25 to 75% of ACN+0.1% TFA in water+0.1% TFA) afforded L6Orn-carbonate-diamino-(mPEG₃₇)₂ (10.20 mg, 1.90 μmol, 100% UV purity, 26% yield) as a white powder after freeze-drying. UPLC-MS (method 5): Rt=1.37 min, m/z=1066 [M+5H]⁵⁺, 889 [M+6H]⁶⁺.

### Preparation of L6Orn-carbonate-PEG₅-PDB-DM4

A saturated aqueous NaHCO₃ solution (83 μL) was added to a mixture of DM4 (2.83 mg, 3.6 μmol, 1.0 eq.) and SPDB (1.18 mg, 3.6 μmol, 1.0 eq.) in DMA (0.6 mL) at rt. After stirring at rt for 10 min, the reaction mixture was added to FcIII-L6Orn-PEG₅-NH₂ (10.1 mg, 3.6 μmol, 1.0 eq.) at rt. After stirring at rt for 50 min, TFA was added until acidic pH was reached. Purification by preparative HPLC (5 to 100% of ACN+0.1% TFA in water+0.1% TFA) afforded L6Orn-carbonate-PEG₅-PDB-DM4 (3.20 mg, 1.1 μmol, 100% UV purity, 29% yield) as a white product after freeze-drying. UPLC-MS (method 4): Rt=2.76 min, m/z=1497 [M-2H]²⁻.

### Preparation of L6Orn(-carbonate-PDB-DM4)-PEG₂₄

A saturated aqueous NaHCO₃ solution (83 μL) was added to a mixture of DM4 (1.22 mg, 1.6 μmol, 1.0 eq.) and SPDB (0.51 mg, 1.6 μmol, 1.0 eq.) in DMA (0.75 mL) at rt. After stirring at rt for 10 min, the reaction mixture was added to L6Orn(-carbonate-NH₂)-PEG₂₄ (5.00 mg, 1.6 μmol, 1.0 eq.) at rt. After stirring at rt for 1 h, TFA was added until acidic pH was reached. Purification by preparative HPLC (5 to 100% of ACN+0.1% TFA in water+0.1% TFA) afforded L6Orn(-carbonate-PDB-DM4)-PEG₂₄ (3.93 mg, 1.0 μmol, 100% UV purity, 64% yield) as a white product after freeze-drying. UPLC-MS (method 4): Rt=2.81 min, m/z=1297 [M+3H]³⁺, 1944 [M-2H]²⁻.

### Preparation of L6Orn(-carbonate-Vedotin)-PEG₂₄

L6Orn(-carbonate-NH₂)-PEG₂₄ (10.0 mg, 3.1 μmol, 1.0 eq.) was added to a mixture of compound 005 (1.49 mg, 3.1 μmol, 1.0 eq.) and DIEA (0.5 μL, 3.1 μmol, 1.0 eq.) in DMF (0.6 mL) at rt. After stirring at rt for 15 min, TFA was added until acidic pH was reached. Purification by preparative HPLC (20 to 80% of ACN+0.1% TFA in water+0.1% TFA) afforded L6Orn(-carbonate-S-Mmt)-PEG₂₄ (8.7 mg, 2.6 μmol, 100% UV purity, 82% yield) as a white product after freeze-drying. UPLC-MS (method 4): Rt=2.96 min, m/z=1684 [M-2H]²⁻.

A degassed mixture of trifluoroacetic acid (40 μL) and DCM (0.96 mL) was added to Vedotin (2.60 mg, 2.0 μmol, 1.0 eq.) at rt. The reaction mixture was stirred at rt for 5 min, then L6Orn(-carbonate-S-Mmt)-PEG₂₄ (8.65 mg, 2.6 μmol, 1.3 eq.) was added followed by DIEA (0.3 μL, 2.0 μmol, 1.0 eq.). After stirring at rt for 10 min, TFA was added until acidic pH was observed. Purification by preparative HPLC (10 to 70% of ACN+0.1% TFA in water+0.1% TFA) afforded L6Orn(-carbonate-Vedotin)-PEG₂₄ (6.70 mg, 1.5 μmol, 100% UV purity, 77% yield) as a white powder after freeze-drying. UPLC-MS (method 4): Rt=2.83 min, m/z=1472 [M+3H]³⁺, 1105 [M+4H]⁴⁺.

### Preparation of PEG₂₀-L6Orn-carbonate-PDB-DM4

A saturated aqueous NaHCO₃ solution (83 μL) was added to a mixture of DM4 (1.28 mg, 1.6 μmol, 1.0 eq.) and SPDB (0.53 mg, 1.6 μmol, 1.0 eq.) in DMA (0.75 mL) at rt. After stirring at rt for 10 min, the reaction mixture was added to PEG₂₀-L6Orn-carbonate-NH₂ (5.00 mg, 1.6 μmol, 1.0 eq.) at rt. After stirring at rt for 1 h, TFA was added until acidic pH was reached. Purification by preparative HPLC (5 to 100% of ACN+0.1% TFA in water+0.1% TFA) afforded PEG₂₀-L6Orn-carbonate-PDB-DM4 (3.45 mg, 0.9 μmol, 100% UV purity, 58% yield) as a white product after freeze-drying. UPLC-MS (method 4): Rt=2.83 min, m/z=1826 [M-2H]²⁻.

### Preparation of PEG₂₀-L6Orn-carbonate-Vedotin

DIEA (0.9 μL, 4.9 μmol, 1.0 eq.) was added to a mixture of PEG₂₀-L6Orn-carbonate-NH₂ (15.0 mg, 4.9 μmol, 1.0 eq.) and compound 005 (2.34 mg, 4.9 μmol, 1.0 eq.) in DMF (1 mL) at rt. After stirring at rt for 15 min, TFA was added until acidic pH was reached. Purification by preparative HPLC (20 to 80% of ACN+0.1% TFA in water+0.1% TFA) afforded PEG₂₀-L6Orn-carbonate-S-Mmt (13.9 mg, 4.4 μmol, 100% UV purity, 90% yield) as a white product after freeze-drying. UPLC-MS (method 4): Rt=2.97 min, m/z=1567 [M-2H]²⁻.

A degassed mixture of trifluoroacetic acid (10 μL) and DCM (0.49 mL) was added to PEG₂₀-L6Orn-carbonate-S-Mmt (6.60 mg, 2.1 μmol, 1.3 eq.) at rt. The reaction mixture was stirred at rt for 5 min, then Vedotin (2.20 mg, 1.6 μmol, 1.0 eq.) was added followed by DIEA (0.3 μL, 1.6 μmol, 1.0 eq.). After stirring at rt for 25 min, TFA was added until acidic pH was observed. Purification by preparative HPLC (10 to 80% of ACN+0.1% TFA in water+0.1% TFA) afforded PEG₂₀-L6Orn-carbonate-Vedotin (4.50 mg, 1.1 μmol, 100% UV purity, 66% yield) as a white powder after freeze-drying. UPLC-MS (method 4): Rt=2.85 min, m/z=1395 [M+3H]³⁺, 1046 [M+4H]⁴⁺.

### Preparation of PEG₂₄-L6Orn-carbonate-PEG₃-DM4

A saturated aqueous NaHCO₃ solution (83 μL) was added to a mixture of DM4 (4.47 mg, 5.7 μmol, 1.0 eq.) and SPDB (1.87 mg, 5.7 μmol, 1.0 eq.) in DMA (2 mL) at rt. After stirring at rt for 5 min, the reaction mixture was added to mPEG₂₄-L6Orn-carbonate-PEG₃-NH₂ (20.00 mg, 5.7 mmol, 1.0 eq.) at rt. After stirring at rt for 40 min, TFA was added until acidic pH was reached. Purification on C18 (30 g, 20 to 80% of ACN+0.1% TFA in water+0.1% TFA over 12 CV) afforded mPEG₂₄-L6Orn-carbonate-PEG₃-PDB-DM4 (11.7 mg, 3.0 μmol, 100% UV purity, 53% yield) as a white product after freeze-drying. UPLC-MS (method 6): Rt=1.19 min, m/z=1923 [M-2H]²⁻.

### Preparation of PEG₂₄-L6Orn-carbonate-PEG₃-(suc-VC-PAB-MMAE)

DIEA (4.1 μL, 20.0 μmol, 4.0 eq.) was added to a mixture of NHS-suc-NH—VC-PAB-MMAE (8.20 mg, 5.8 μmol, 1.0 eq.) and PEG₂₄-L6Orn-carbonate-PEG₃-NH₂ (19.6 mg, 5.6 μmol, 0.96 eq.) in DMF (1 mL) at rt. After stirring at rt for 1 h, PEG₂₄-L6Orn-carbonate-PEG₃-NH₂ (4.08 mg, 1.2 μmol, 0.2 eq.) was added to the reaction mixture. After stirring at rt for 1 h, TFA was added until acidic pH was reached. Purification by preparative HPLC (20 to 80% of ACN+0.1% TFA in water+0.1% TFA) was achieved yielding PEG₂₄-L6Orn-carbonate-PEG₃-(suc-NH—VC-PAB-MMAE) (13.2 mg, 3.2 μmol, 100% UV purity, 54% yield) as a white powder after freeze-drying. UPLC-MS (method 6): Rt=1.44 min, m/z=1392 [M+2H]²⁺.

### Preparation of L6Orn-carbonate-suc-VC-PAB-MMAE

DIEA (13 μL, 26.0 μmol, 3.0 eq.) was added to a solution of NHS-suc-NH—VC-PAB-MMAE (12.6 mg, 9.5 μmol, 1.1 eq.) in 450 μL DMF and L6Orn-carbonate-NH₂ (15.8 mg, 8.7 μmol, 1.0 eq.) in 86 μL DMF. After stirring at rt for 2 h, TFA was added until acidic pH was reached. Purification by preparative HPLC (34 to 50% of ACN in water with 0.1% TFA) afforded L6Orn-carbonate-suc-VC-PAB-MMAE (17.1 mg, 5.6 μmol, 99% UV purity, 65% yield) as a white powder after freeze-drying. UPLC-MS (method 2): Rt=2.98 min, m/z=1516 [M+2H]²⁺.

### Preparation of PEG₂₄-L6Orn-carbonate-suc-VC-PAB-MMAE

DIEA (20 μL, 114.0 μmol, 20.0 eq.) was added to NHS-suc-NH—VC-PAB-MMAE (7.4 mg, 5.6 μmol, 1.0 eq.) in 235 μL DMF and PEG₂₄-L6Orn-carbonate-NH₂ (16.5 mg, 5.6 μmol, 1.0 eq.) in 150 μL DMF. After stirring at rt for 1 h, TFA was added until acidic pH was reached. Purification by preparative HPLC (35 to 55% of ACN in water with 0.1% TFA) afforded PEG₂₄-L6Orn-carbonate-suc-VC-PAB-MMAE (8.7 mg, 2.1 μmol, 99% UV purity, 37% yield) as a white powder after freeze-drying. UPLC-MS (method 2): Rt=3.03 min, m/z=1378 [M+2H]²⁺.

### Preparation of L6Orn-carbonate-PNU

DIEA (11.5 μL, 65.8 μmol, 4.0 eq.) was added to a solution of L6Orn-carbonate-NH₂ (30.0 mg, 16.4 μmol, 1.0 eq.) and PNU-OPFP (compound 027) (14.0 mg, 16.4 μmol, 1.0 eq.) in DMF (1 mL) at rt. After stirring at rt for 2.5 h, TFA (5 μL) was added to the reaction mixture at rt. Purification by preparative HPLC (20 to 60% of ACN in water) afforded L6Orn-carbonate-PNU (18.9 mg, 5.8 μmol, 77% UV purity, 35% yield) as an orange powder after freeze-drying. UPLC-MS (method 3): Rt=1.47 min, m/z=1247 [M+2H]²⁺, 1244 [M-2H]²⁻.

### Preparation of PEG₂₄-L6Orn-carbonate-PNU

DIEA (12 μL, 68.7 μmol, 4.0 eq.) was added to a solution of PNU-OPFP (15.5 mg, 18.2 μmol, 1.05 eq.) and PEG₂₄-L6Orn-carbonate-NH₂ (49.5 mg, 17.2 μmol, 1.0 eq.) in DMF (2 mL). After stirring at rt for 1 h, TFA was added until acidic pH was reached. Purification by preparative HPLC (30 to 60% of ACN in water) afforded PEG₂₄-L6Orn-carbonate-PNU (24.3 mg, 5.7 μmol, 83% UV purity, 33% yield) as an orange powder after freeze-drying. UPLC-MS (method 3): Rt=1.52 min, m/z=1775 [M+2H]²⁺, 1773 [M-2H]²⁻.

### Preparation of PEG₂₀-L6Orn-carbonate-multi-(DM1)₂

A degassed mixture of trifluoroacetic acid (10 μL) and DCM (490 μL) was added to PEG₂₀-L6Orn-carbonate-S-Mmt (5.18 mg, 1.7 μmol, 1.2 eq.) at rt. The reaction mixture was stirred at rt for 5 min, then compound 016 (6.00 mg, 1.4 μmol, 1.0 eq.) was added followed by DIEA (20 μL, 90 μmol, 70 eq.). After stirring at rt for 25 min, TFA was added until acidic pH was reached. The reaction mixture was filtered.

Purification by preparative HPLC (10 to 80% of ACN+0.1% TFA in water+0.1% TFA) afforded PEG₂₀-L6Orn-carbonate-multi-(DM1)₂ (3.50 mg, 0.40 μmol, 90% UV purity, 32% yield) as a white powder after freeze-drying. UPLC-MS (method 4): Rt=2.52 min, m/z=1807 [M+4H]⁴⁺, 1442 [M+5H]⁵⁺, 1204 [M+6H]⁶⁺.

### Preparation of L6Orn-carbonate-multi-(DM1)₂-PEG₁s

A degassed mixture of trifluoroacetic acid (10 μL) and DCM (490 μL) was added to L6Orn-carbonate-S-Mmt (3.27 mg, 1.5 μmol, 1.2 eq.) at rt. The reaction mixture was stirred at rt for 5 min, then compound 024 (6.35 mg, 1.2 μmol, 1.0 eq.) was added followed by DIEA (20 μL, 87.3 μmol, 70 eq.). After stirring at rt for 10 min, TFA was added until acidic pH was reached. The reaction mixture was filtered. Purification by preparative HPLC (10 to 80% of ACN+0.1% TFA in water+0.1% TFA) afforded L6Orn-carbonate-multi-(DM1)₂-PEG₁₅ (3.70 mg, 0.50 μmol, 100% UV purity, 42% yield) as a white powder after freeze-drying. UPLC-MS (method 4): Rt=2.43 min, m/z=1751 [M+4H]⁴⁺, 1398 [M+5H]⁵⁺.

### Preparation of L6Orn-carbonate-multi-(DM1)-K(PEG₂₄)-(DM1)

A degassed mixture of trifluoroacetic acid (12 μL) and DCM (564 μL) was added to L6Orn-carbonate-S-Mmt (3.52 mg, 1.6 μmol, 1.2 eq.) at rt. The reaction mixture was stirred at rt for 5 min, then compound 026 (6.00 mg, 1.3 μmol, 1.0 eq.) was added followed by DIEA (16 μL, 94 μmol, 70 eq.). After stirring at rt for 10 min, TFA was added until acidic pH was reached. Purification by preparative HPLC (10 to 80% ACN+0.1% TFA in water+0.1% TFA) afforded L6Orn-carbonate-multi-(DM1)-K(PEG₂₄)-(DM1) (6.25 mg, 1.0 μmol, 98% UV purity, 73% yield) as white powder after freeze-drying. UPLC-MS (method 4): Rt=3.18 min, m/z=1542 [M+4H]⁴⁺.

### Preparation of RGNCAYHK(NHS-N₃-DBCO-VC-PAB-MMAE)GQIIWCTYH

DBCO-VC-PAB-MMAE (6.6 mg, 4.7 μmol, 1.4 eq.) was added to a solution of RGNCAYHK(NHS-N₃)GQIIWCTYH (8.0 mg, 3.3 μmol, 1.0 eq.) in DMF (190 μL). After stirring at rt for 4 h. Purification by preparative HPLC (30 to 50% ACN+0.1% TFA in water+0.1% TFA) afforded RGNCAYHK(NHS-N₃-DBCO-VC-PAB-MMAE)GQIIWCTYH (1.20 mg, 0.3 μmol, 96% UV purity, 10% yield) as a white powder after freeze-drying. UPLC-MS (method 2): Rt=2.84 min, m/z=1287.7 [M+2H]²⁺.

### Preparation of RGNCAYHOrn(NHS-N₃-DBCO-VC-PAB-MMAE)GQIIWCTYH

DBCO-VC-PAB-MMAE (6.3 mg, 4.5 μmol, 1.4 eq.) was added to a solution of RGNCAYHOrn(NHS-N₃)GQIIWCTYH (7.6 mg, 3.1 μmol, 1.0 eq.) in DMF (140 μL) and stirred overnight at rt. Purification by preparative HPLC (30 to 50% ACN+0.1% TFA in water+0.1% TFA) afforded RGNCAYHOrn(NHS-N₃-DBCO-VC-PAB-MMAE)GQIIWCTYH (2.10 mg, 0.3 μmol, 96% UV purity, 10% yield) as a white powder after freeze-drying. UPLC-MS (method 2): Rt=2.84 min, m/z=1287.7 [M+2H]²⁺.

### Preparation of RGNCAYHK(NHS-PEG₁-VC-PAB-MMAE)GQIIWCTYH

6-Cl-HOBt (0.4 mg, 3.0 μmol, 1.0 eq) followed by EDCl (0.5 mg, 3.0 μmol, 1.0 eq.) were added to a solution of compound 022 (4.6 mg, 3.6 μmol, 1.2 eq.) in DMF (73 μL) at rt. After stirring at rt for 2 min, RGNCAYHK(SuOH)GQIIWCTYH (7.0 mg, 3.0 μmol, 1.0 eq.) in DMF (0.15 mL) was added to the reaction mixture. The reaction was stirring at rt for 4h. Purification by preparative HPLC (26 to 46% ACN+0.1% TFA in water+0.1% TFA) afforded RGNCAYHK(NHS-PEG₁-VC-PAB-MMAE)GQIIWCTYH (1.60 mg, 0.5 μmol, 96% UV purity, 15% yield) as a white powder after freeze-drying. UPLC-MS (method 2): Rt=2.59 min, m/z=1185 [M+2H]²⁺.

### Preparation of RGNCAYHK(carbonate-N₃-DBCO-VC-PAB-MMAE)GQIIWCTYH

DBCO-VC-PAB-MMAE (9.1 mg, 6.5 μmol, 64.5 μL, 3.9 eq.) was added to a solution of RGNCAYHK(carbonate-N₃)GQIIWCTYH (6.6 mg, 2.9 μmol, 1.0 eq.) in DMF (70 μL). After stirring at rt for 4 h. Purification by preparative HPLC (29 to 49% ACN+0.1% TFA in water+0.1% TFA) afforded RGNCAYHK(carbonate-N₃-DBCO-VC-PAB-MMAE)GQIIWCTYH (4.40 mg, 1.2 μmol, 100% UV purity, 41% yield) as a white powder after freeze-drying. UPLC-MS (method 2): Rt=2.81 min, m/z=1245 [M+2H]²⁺.

### Preparation of RGNCAYHK(carbonate-suc-VC-PAB-MMAE)GQIIWCTYH

DIEA (66 μL, 379 μmol, 53.0 eq.) was added to NHS-suc-NH—VC-PAB-MMAE (11.4 mg, 8.6 μmol, 1.2 eq.) in 430 μL DMF and RGNCAYHK(carbonate-NH₂)GQIIWCTYH (16.8 mg, 7.2 μmol, 1.0 eq.) in 150 μL DMF. After stirring at rt for 3.5 h, TFA was added until acidic pH was reached. Purification by preparative HPLC (26 to 42% of ACN in water with 0.1% TFA) afforded RGNCAYHK(carbonate-suc-VC-PAB-MMAE)GQIIWCTYH (11.8 mg, 3.3 μmol, 99% UV purity, 46% yield) as a white powder after freeze-drying. UPLC-MS (method 2): Rt=2.62 min, m/z=1183 [M+2H]²⁺.

The MS characterization of peptide conjugates described in Example 7 are shown in the table below. The MS data was obtained by ESI measured in a positive mode.

TABLE 10
Characterization of peptide reactive conjugates

| | Cal- | | | | | |
|---|---|---|---|---|---|---|
| | culated | Cal- | | Retention | | UPLC- |
| | Exact | culated | Found | time | Purity | MS |
| Compound | Mass | m/z | m/z | (min) | (%) | method |
| L6Orn- | 2418.70 | 1210.35 | 1210 | 2.11 | 90 | 1 |
| carbonate- | | | | | | |
| DTPA | | | | | | |
| L6Orn- | 2351.64 | 1176.82 | 1176.4 | 2.46 | 96 | 2 |
| carbonate- | | | | | | |
| PCTA | | | | | | |
| L6Orn- | 2181.4 | 1091.7 | 1091.4 | 2.303 | 87 | 2 |
| carbonate- | | | | | | |
| NODA-GA | | | | | | |
| L6Orn- | 2282.53 | 761.84 | 761 | 1.85 | 74 | 4 |
| carbonate- | | | | | | |
| DOTA-GA | | | | | | |
| L6Orn- | 2576.70 | 1289.35 | 1289.4 | 2.562 | 83 | 2 |
| carbonate- | | | | | | |
| DFO | | | | | | |
| L6Orn- | 2756.03 | 919.68 | 919 | 2.34 | 84 | 4 |
| carbonate- | | | | | | |
| triazole-dbco- | | | | | | |
| DFO | | | | | | |
| L6Orn- | 2624.94 | 1313.47 | 1313 | 2.11 | 98 | 4 |
| carbonate- | | | | | | |
| maleimide- | | | | | | |
| DFO | | | | | | |
| L6Orn- | 2182.12 | 1092.06 | 1091.61 | 1.9 | 96 | 1 |
| carbonate- | | | | | | |
| FAM | | | | | | |
| L6Orn- | 2313.66 | 1157.83 | 1157 | 2.29 | 91 | 4 |
| carbonate-TZ | | | | | | |
| L6Orn- | 1976.24 | 989.12 | 988 | 2.75 | 80 | 4 |
| carbonate- | | | | | | |
| TCO | | | | | | |
| L6Orn- | 1907.10 | 954.55 | 954 | 2.16 | 94 | 4 |
| carbonate-N₃ | | | | | | |
| L6Orn- | 2111.36 | 1056.68 | 1056 | 2.79 | 97 | 4 |
| carbonate- | | | | | | |
| DBCO | | | | | | |
| L6Orn- | 2000.26 | 1001.13 | 1000.8 | 2.903 | 95 | 2 |
| carbonate- | | | | | | |
| BCN | | | | | | |
| L6Orn- | 2134.35 | 1068.18 | 1067 | 2.07 | 91 | 4 |
| carbonate- | | | | | | |
| maleimide | | | | | | |
| L6Orn- | 2781.58 | 1391.79 | 1392 | 2.8 | 96 | 4 |
| carbonate- | | | | | | |
| MCC-DM1 | | | | | | |
| L6Orn- | 2705.53 | 1353.77 | 1353 | 2.99 | 99 | 4 |
| carbonate- | | | | | | |
| SPDB-DM4 | | | | | | |
| L6Orn- | 3747.25 | 1874.63 | 1875 | 3.49 | 87 | 4 |
| carbonate- | | | | | | |
| diaminoxylene | | | | | | |
| (-SPDB- | | | | | | |
| DM4)₂ | | | | | | |
| L6Orn- | 3229.74 | 1077.58 | 1077 | 3.01 | 94 | 4 |
| carbonate- | | | | | | |
| Vedotin | | | | | | |
| L6Orn- | 4592.46 | 1531.82 | 1531 | 3.49 | 97 | 4 |
| carbonate- | | | | | | |
| (Vedotin)₂ | | | | | | |
| L6Orn- | 3586.39 | 1794.20 | 1794 | 2.06 | 98 | 4 |
| carbonate- | | | | | | |
| multi-DM1 | | | | | | |
| L6Orn- | 6272.81 | 1569.2 | 1568.8 | 2.59 | 97 | 4 |
| carbonate- | | | | | | |
| multi-(DM1)₂ | | | | | | |

### Example 8: Preparation of Other Payload-Containing Reactive Conjugates

The Fc-binding vectors prepared in Example 1 were converted into reactive conjugates by coupling of compound 008 or payload-thioester/ester to the amino group of the side chain of the respective Fc-binding vectors. The structures of the payload-thioester/ester-containing reactive conjugates prepared in Example 8 are shown in the table below.

TABLE 11
Structures of payload-thioester/ester-containing reactive conjugates

| Compound | Structure |
|---|---|
| A3K-thioester- DOTA (A3K + 008) | 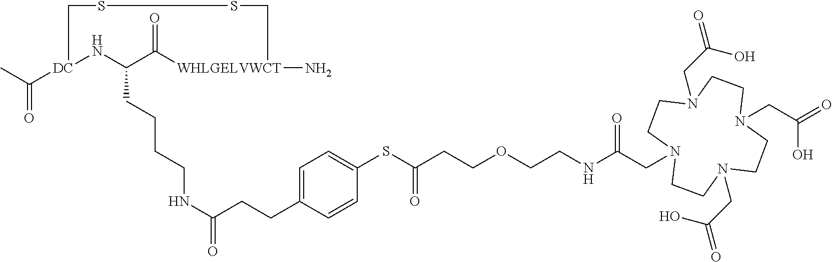 |
| L6Dap- thioester- DOTA (L6Dap + 0085) | 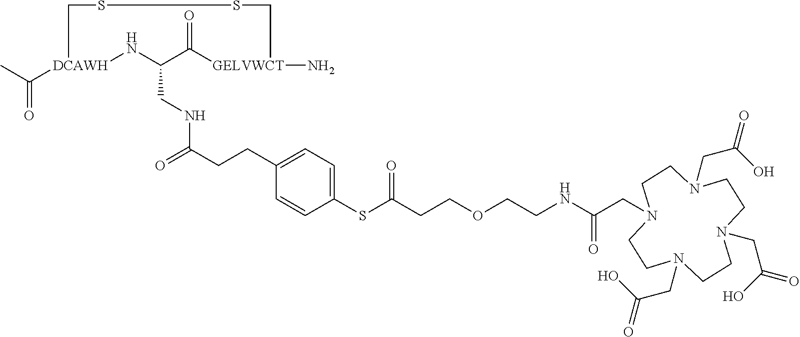 |
| L6Dap(-thio- ester- DOTA)-E8Q (L6Dap-E8Q + 008) | 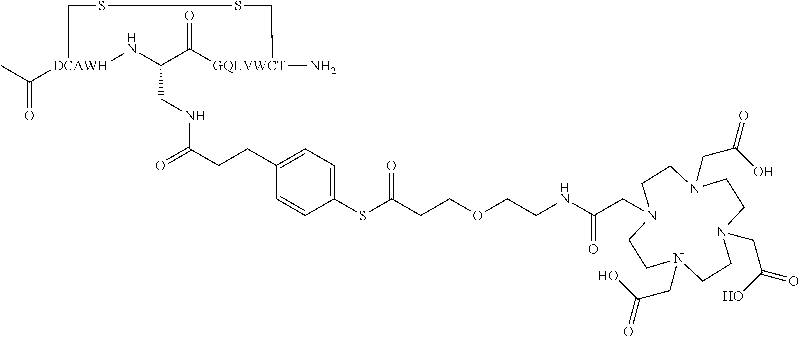 |
| L6Dap- thioester- DTPA | 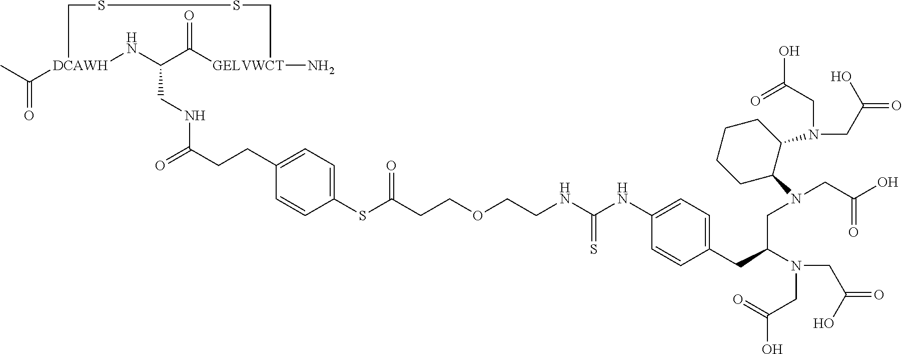 |
| L6Dap- thioester- PCTA | 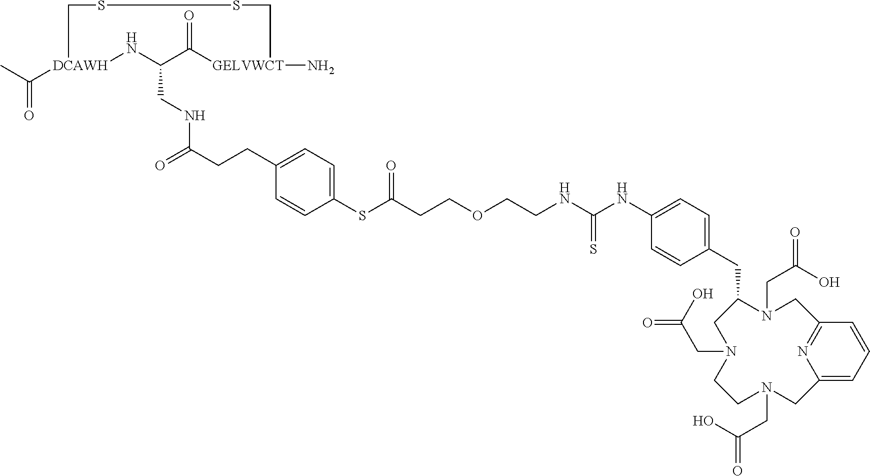 |
| L6Dap- thioester-Bn- NODA-GA | 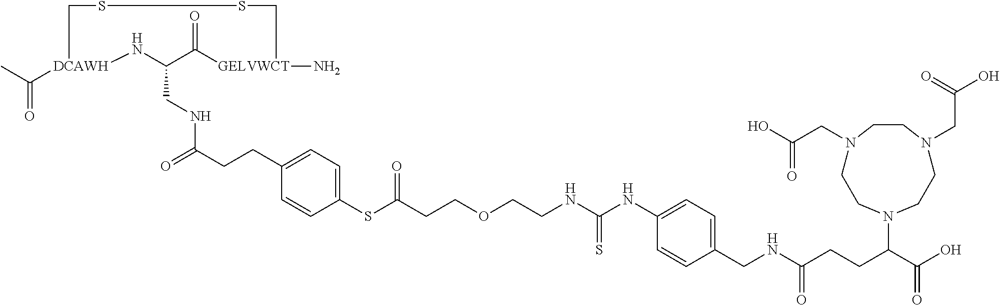 |
| L6Dap- thioester-Bn- NOTA | 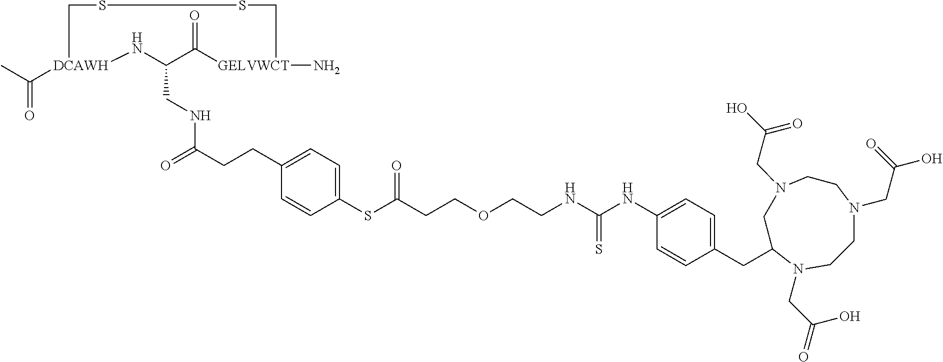 |
| L6Dap- thioester- DOTA-GA | 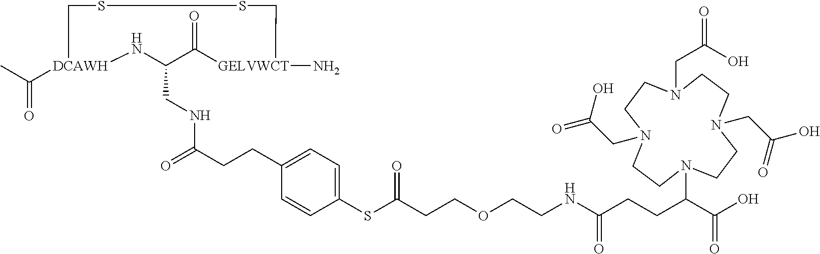 |
| L6Dap- thioester-N₃ | 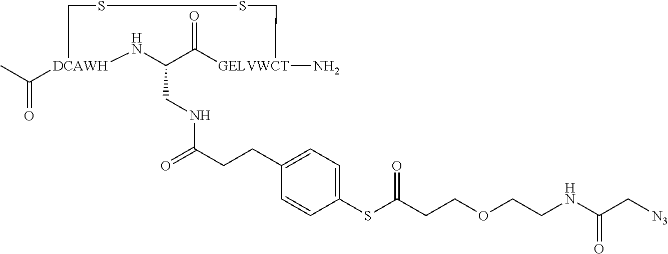 |
| L6Dap- thioester- triazole-dbco- DFO | 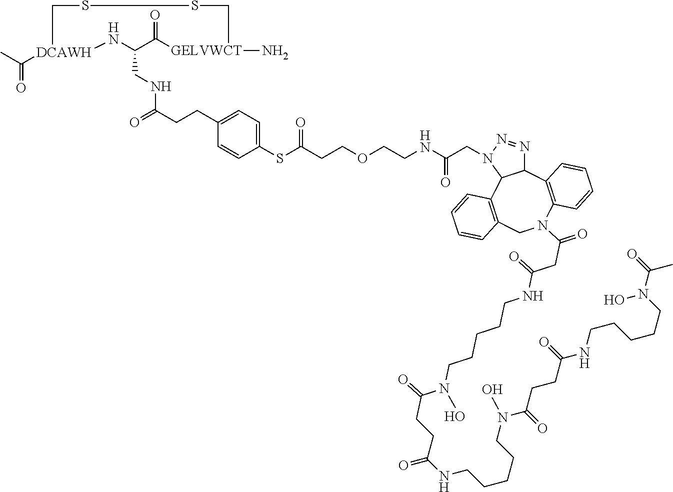 |
| L6Dap- thioester-FITC | 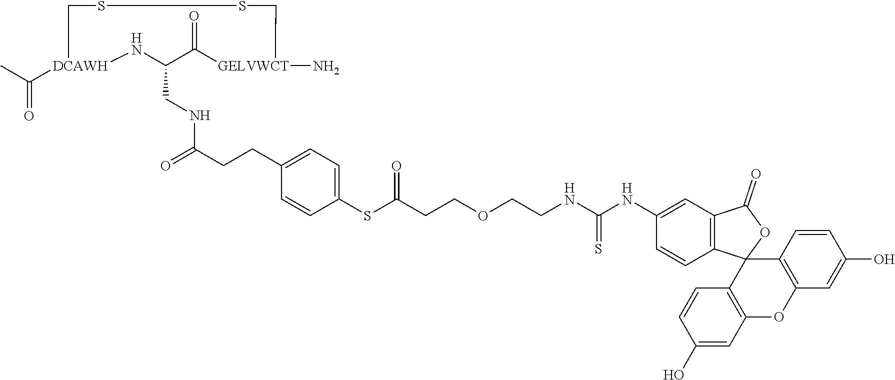 |
| L6Dap- thioester- MCC-DM1 | 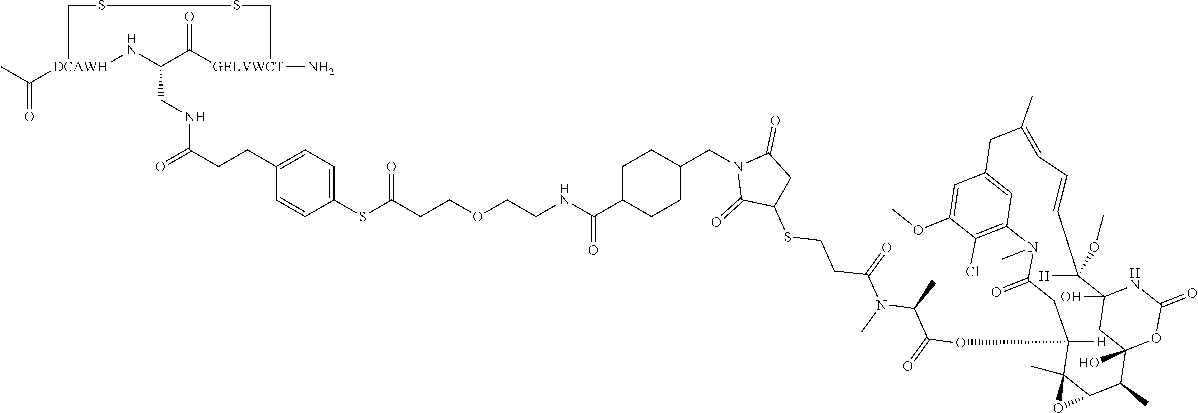 |
| L6Dap- thioester- SPDB-DM4 | 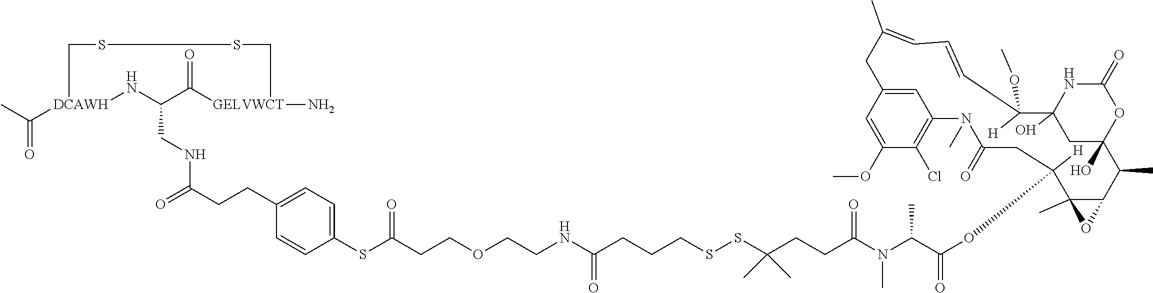 |
| L6Dab- thioester- DOTA (L6Dab + 008) | 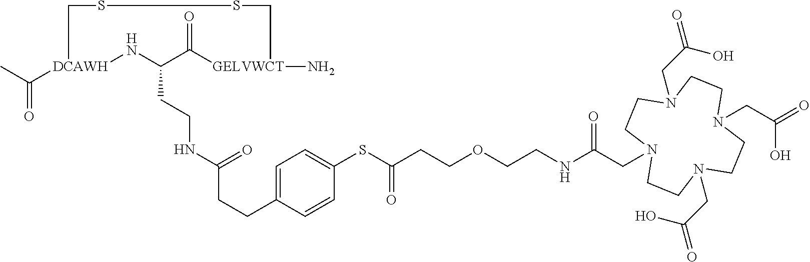 |
| L6Orn- thioester- DOTA (L60rn + 008) | 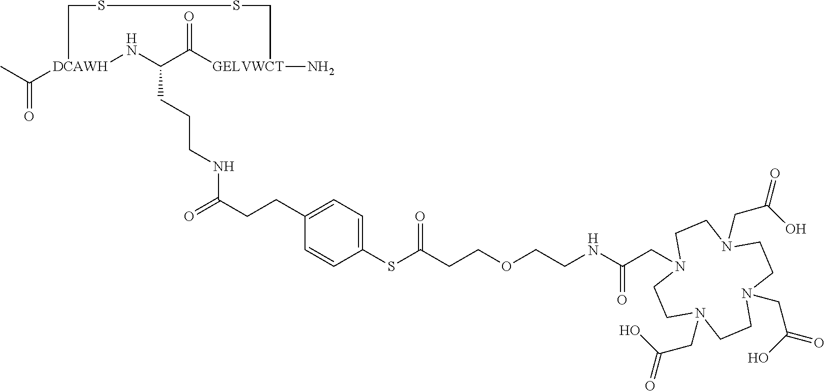 |
| L6K-thioester- DOTA (L6K + 008) | 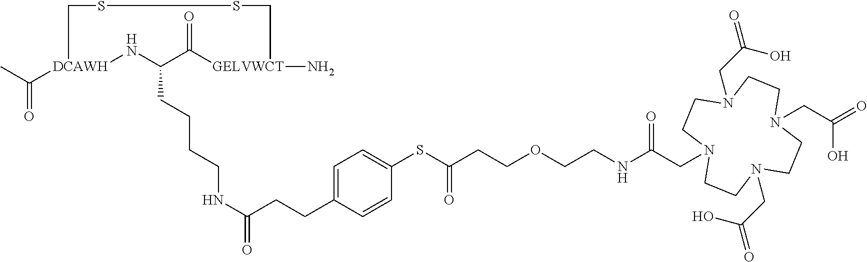 |
| L6Orn-ester- DOTA | 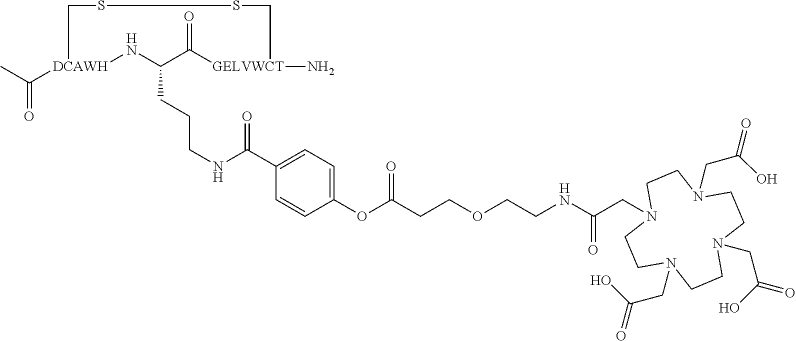 |
| L6Orn-ester- DTPA | 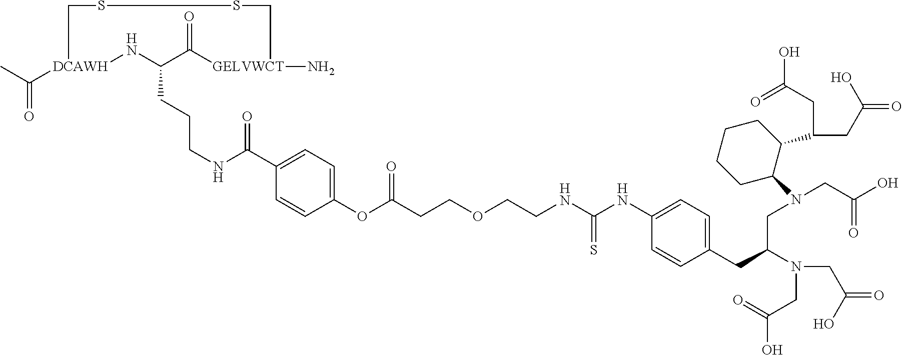 |
| L6Orn-ester- PCTA | 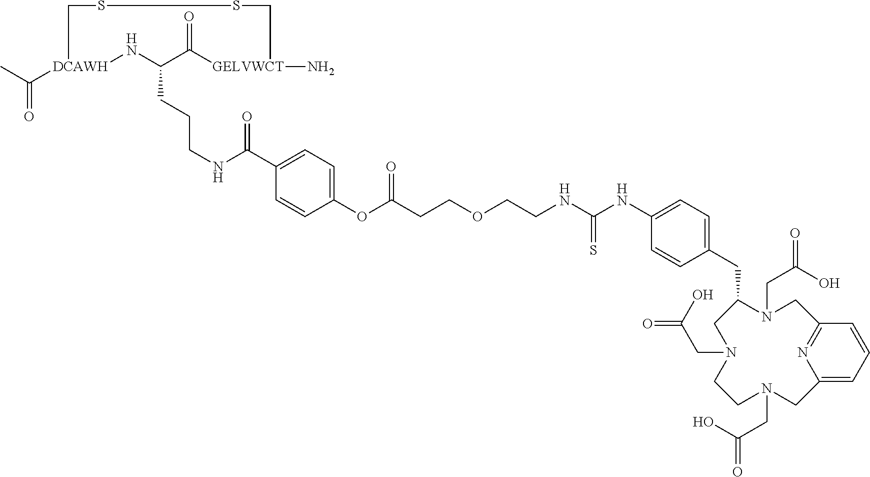 |
| L6Orn-ester- Bn-NOTA | 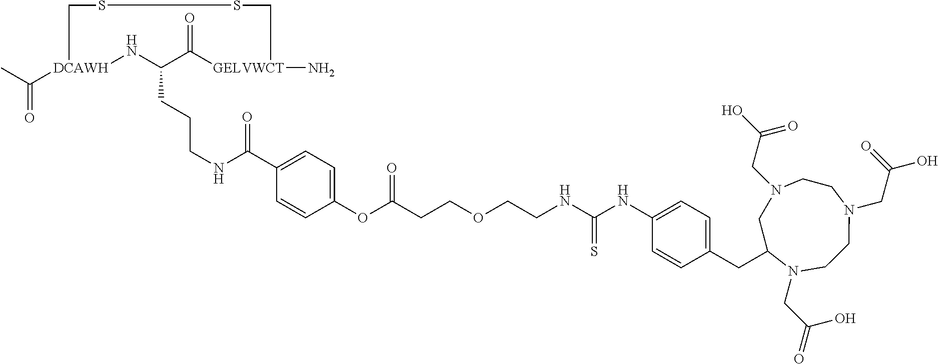 |
| L6Orn-ester- FITC | 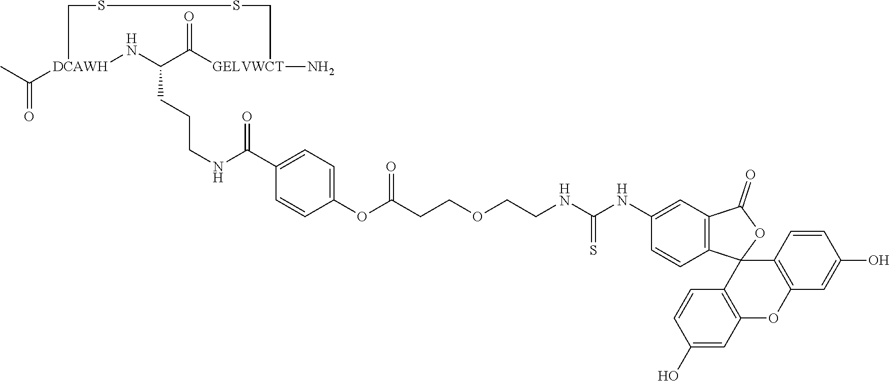 |
| L6Orn-ester- N₃ | 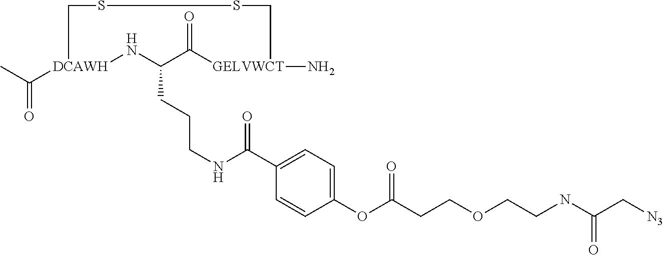 |
| L6Dap-(CH₂)₂- ester-DOTA | 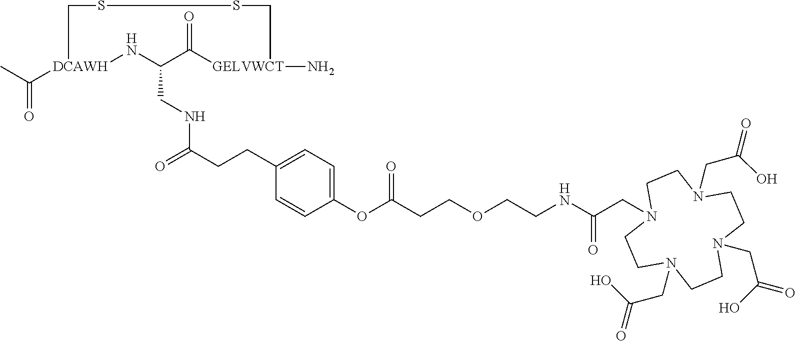 |
| L6Dap-(CH₂)₂- ester-DTPA | 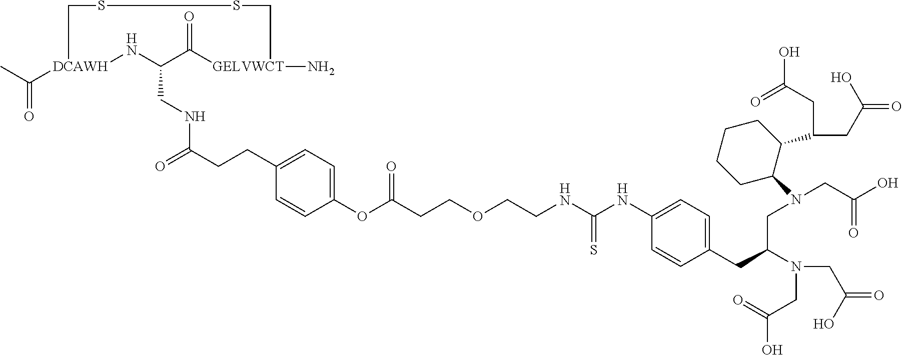 |

### Preparation of A3K/L6Dap-E8Q/L6Dab/L6Orn/L6K-thioester-DOTA

HATU·HPF₆ (1.3 eq.) was added to a solution of compound 008 (1.4 eq.) and stirred for 1 min, followed by the addition of DIEA (3.0 eq.) at rt. After 3 min stirring at rt, the pre-activated compound 008 was added to A3K/L6Dap-E8Q/L6Dab/L6Orn/L6K peptides (1.0 eq.) and stirred at rt for 1−3 h. Completion of the reaction was monitored by UPLC-MS. The reactive conjugates were precipitated with cold diethyl ether.

Subsequently, the tert-butyl protecting groups of the DOTA moiety were removed by treatment with TFA/TIS/water (95/2.5/2.5, v/v/v) over 1-3 hours at rt, followed by precipitation with cold diethyl ether and purification by HPLC (as in Example 6).

### Preparation of L6Dap-thioester-NH₂

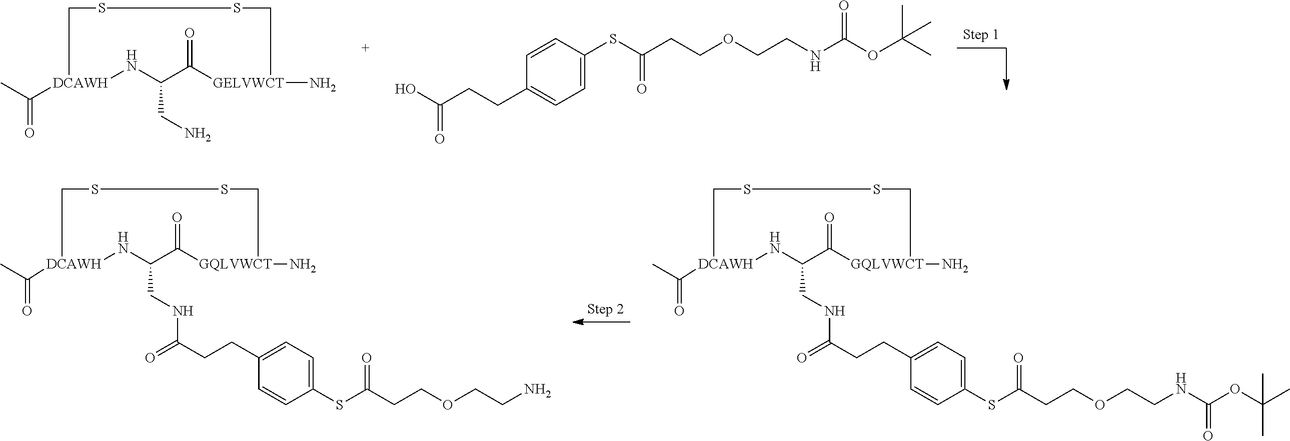

Step 1. HATU·HPF₆ (10.3 mg, 26.1 μmol, 1.2 eq.) followed by DIEA (9 μL, 52.2 μmol, 2.4 eq.) were added to a solution of 3[4[3[2(tertbutoxycarbonylamino)ethoxy]propanoylsulfanyl]phenyl]propanoic acid (9.1 mg, 21.7 μmol, 1.0 eq.) in DMF (0.22 mL) at rt. After stirring at rt for 5 min, L6Dap-thioester-NH₂ (40.3 mg, 26.1 μmol, 1.2 eq.) was added to the reaction mixture. After stirring at rt for 55 min, TFA was added until acidic pH was reached. Purification by reversed phase preparative HPLC (eluent: 5 to 100% of ACN+0.1% FA in water+0.1% FA) afforded L6Dap-thioester-NHBoc (21.8 mg, 10.8 μmol, 95% UV purity, 49% yield) as a white solid after freeze-drying. UPLC-MS (method 4): Rt=2.63 min, m/z=912 [M-Boc+2H]²⁺, 1924 [M+H]⁺, 961 [M-2H]²⁻.

Step 2. TFA (1 mL) was added to a solution of L6Dap-thioester-NHBoc (21.8 mg, 11.3 μmol, 1.0 eq.) at rt. The reaction mixture was stirred at rt for 7 min then concentrated in vacuo. Water (0.5 mL) and ACN (0.5 mL) were added and the mixture was freeze-dried to afford L6Dap-thioester-NH₂ (23.0 mg, 11.3 μmol, 90% UV purity, quantitative yield) as a white powder after freeze-drying. UPLC-MS (method 4): Rt=1.90 min, m/z=912 [M+2H]²⁺, 1824 [M+H]+.

### Preparation of L6Dap-thioester-DOTA

DIEA (3.1 μL, 18 μmol, 4.0 eq.) was added to a solution of 1,4,7,10-tetraazacyclododecane-1,4,7,10-tetraacetic acid mono-N-hydroxysuccinimide ester HPF₆ TFA (3.4 mg, 4.5 μmol, 1.0 eq.) and L6Dap-thioester-NH₂ (8.2 mg, 4.5 μmol, 1.0 eq.) in DMF (0.3 mL) at rt. The reaction mixture was stirred at rt for 1 h then two drops of TFA were added. Purification by reversed phase preparative HPLC (5-50% ACN+0.1% FA in water+0.1% FA) gave L6Dap-thioester-DOTA (1.7 mg, 0.8 μmol, 99% UV purity, 17% yield) as a white powder after freeze-drying. UPLC-MS (method 4): Rt=2.95 min, m/z=737 [M+3H]³⁺, 1104 [M-2H]²⁻.

### Preparation of L6Dap-thioester-DTPA

DIEA (3.1 μL, 17.5 μmol, 4.0 eq.) was added to a solution of L6Dap-thioester-NH₂ (8.0 mg, 4.4 μmol, 1.0 eq.) and p-SCN-Bn-CHX-A″-DTPA·3HCl (3.1 mg, 4.4 μmol, 1.0 eq.) in DMF (0.2 mL) at rt. The reaction mixture was stirred at rt for 1 h then two drops of TFA were added. Purification by preparative HPLC (5-50% ACN+0.1% FA in water+0.1% FA) afforded L6Dap-thioester-DTPA (0.13 mg, 0.04 μmol, 80% UV purity, 1% yield) as a white powder after freeze-drying. UPLC-MS (method 4): Rt=2.30 min, m/z=807 [M+3H]³⁺, 1210 [M+2H]²⁺.

### Preparation of L6Dap-thioester-PCTA

DIEA (1.9 μL, 11.0 μmol, 4.0 eq.) was added to a mixture of L6Dap-thioester-NH₂ (5.0. mg, 2.7 μmol, 1.0 eq.) and p-SCN-Bn-PCTA.3HCl (1.75 mg, 2.7 μmol, 1.0 eq.) in DMF (0.1 mL) at rt. After stirring at rt for 1 h, TFA was added until acidic pH was reached. Purification on C18 (12 g, 20 to 80% ACN+0.1% TFA in H₂O+0.1% TFA over 12 CV) afforded L6Dap-thioester-PCTA (0.5 mg, 0.21 μmol, 90% UV purity, 8% yield) as a white powder after freeze-drying. UPLC-MS (method 4): Rt=2.19 min, m/z=784 [M+3H]³⁺, 1174 [M-2H]²⁻.

### Preparation of L6Dap-thioester-Bn-NODA-GA

DIEA (1.3 μL, 7.5 μmol, 4.0 eq.) was added to a mixture of L6Dap-thioester-NH₂ (3.4 mg, 1.9 μmol, 1.0 eq.) and p-SCN-Bn-NODA-GA (0.97 mg, 1.9 μmol, 1.0 eq.) in DMF (0.3 mL) at rt. After stirring at rt for 2 h, TFA was added until acidic pH was reached. Purification on C18 (27 to 37% ACN+0.1% TFA in H₂O+0.1% TFA) afforded L6Dap-thioester-Bn-NODA-GA (0.1 mg, 42 nmol, 95% UV purity, 2.6% yield) as a white powder after freeze-drying. UPLC-MS (method 2): Rt=2.95 min, m/z=1173.3 [M+2H]²⁺.

### Preparation of L6Dap-thioester-Bn-NOTA

DIEA (1.9 μL, 11.0 μmol, 4.0 eq.) was added to a mixture of L6Dap-thioester-NH₂ (5.0 mg, 2.7 μmol, 1.0 eq.) and of p-SCN-Bn-NOTA (1.53 mg, 2.7 μmol, 1.0 eq.) in DMF (0.3 mL) at rt. After stirring at rt for 30 min, TFA was added until acidic pH was reached. Purification on C18 (12 g, 20 to 80% of ACN+0.1% TFA in water+0.1% TFA over 12 CV) afforded L6Dap-thioester-Bn-NOTA (1.1 mg, 0.5 μmol, UV purity 100%, 18% yield) as a white powder after freeze-drying. UPLC-MS (method 4): Rt=2.23 min, m/z=1138 [M+2H]²⁺, 1136 [M-2H]²⁻.

### Preparation of L6Dap-thioester-DOTA-GA

DIEA (1.9 μL, 11.0 μmol, 4.0 eq.) was added to a solution of DOTA-GA(OtBu)₄ (2.1 mg, 3.0 μmol, 1.1 eq.) and HATU·HPF₆ (1.15 mg, 3.0 μmol, 1.1 eq.) in DMF (0.3 mL) at rt. After stirring at rt for 5 min, the reaction mixture was added to L6Dap-thioester-NH₂ (5.0 mg, 2.7 μmol, 1.0 eq.) at rt. After stirring at rt for 10 min, TFA was added until acidic pH was reached. Purification on C18 (12 g, 20 to 80% of ACN+0.1% TFA in water+0.1% TFA over 12 CV) afforded L6Dap-thioester-DOTA-GA(OtBu)₄ (3.9 mg, 1.5 μmol, UV purity 98%, 56% yield) as a white powder after freeze-drying. The latter compound was dissolved in DCM (0.5 mL) and TFA (0.5 mL) and stirred at rt. After stirring at rt for 21 h, the reaction mixture was concentrated in vacuo then purification on C18 (12 g, 20 to 50% of ACN+0.1% TFA in water+0.1% TFA over 10 CV) afforded L6Dap-thioester-DOTA-GA (1.5 mg, 0.4 μmol, UV purity 61%, 27% yield) as a white powder after freeze-drying. UPLC-MS (method 4): Rt=1.96 min, m/z=761 [M+3H]³⁺, 1140 [M-2H]²⁻.

### Preparation of L6Dap-thioester-triazole-dbco-DFO

DBCO-DFO (1.53 mg, 1.8 μmol, 1.0 eq.) was added to a mixture of L6Dap-thioester-N₃ (3.5 mg, 1.8 μmol, 1.0 eq.) in DMF (0.3 mL) at rt. After stirring at rt for 75 min, TFA was added until acidic pH was reached. Purification on C18 (12 g, 20 to 80% of ACN+0.1% TFA in water+0.1% TFA over 12 CV) afforded L6Dap-thioester-triazole-dbco-DFO (3.4 mg, 1.2 μmol, UV purity 99%, 67% yield) as a white powder after freeze-drying. UPLC-MS (method 4): Rt=2.46 min, m/z=919 [M+3H]³⁺, 1376 [M-2H]²⁻.

### Preparation of L6Dap-thioester-N₃

DIEA (7.6 μL, 43.8 μmol, 4.0 eq.) was added to a mixture of L6Dap-thioester-NH₂ (20.0 mg, 11.0 μmol, 1.0 eq.) and (2,5-dioxopyrrolidin-1-yl) 2-azidoacetate (2.17 mg, 11.0 μmol, 1.0 eq.) in DMF (1.2 mL) at rt. After stirring at rt for 75 min, TFA was added until acidic pH was reached. Purification on C18 (12 g, 20 to 80% of ACN+0.1% TFA in water+0.1% TFA over 12 CV) afforded L6Dap-thioester-N3 (3.5 mg, 1.8 μmol, UV purity 92%, 15% yield) as a white powder after freeze-drying. UPLC-MS (method 4): Rt=2.30 min, m/z=954 [M+2H]²⁺, 952 [M-2H]²⁻.

### Preparation of L6Dap-thioester-FITC

DIEA (3.1 μL, 17.5 μmol, 4.0 eq.) was added to a solution of L6Dap-thioester-NH₂ (8.0. mg, 4.4 μmol, 1.0 eq.) and 5-FITC (1.7 mg, 4.4 μmol, 1.0 eq.) in DMF (0.2 mL) at rt. The reaction mixture was stirred at rt for 1 h then two drops of TFA were added. Purification by preparative HPLC (5−50% ACN+0.1% FA in water+0.1% FA) afforded L6Dap-thioester-FITC (4.4 mg, 1.6 μmol, 83% UV purity, 38% yield) as a yellow powder after freeze-drying. UPLC-MS (method 4): Rt=2.58 min, m/z=738 [M+3H]³⁺, 1107 [M+2H]²⁺.

### Preparation of L6Dap-thioester-MCC-DM1

DIEA (1.3 μL, 7.5 μmol, 4.0 eq.) was added to a mixture of L6Dap-thioester-NH₂ (3.4 mg, 1.9 μmol, 1.0 eq.) and DM1-SMCC (2.0 mg, 1.9 μmol, 1.0 eq.) in DMF (0.3 mL) at rt. After stirring at rt for 2 h, TFA was added until acidic pH was reached.

Purification on C18 (35 to 47% ACN+0.1% TFA in H₂O+0.1% TFA) afforded L6Dap-thioester-MCC-DM1 (0.27 mg, 96 nmol, 92% UV purity, 5% yield) as a white powder after freeze-drying. UPLC-MS (method 2): Rt=3.06 min, m/z=1389.6 [M-2H]²⁻.

### Preparation of L6Dap-thioester-SPDB-DM4

A saturated aqueous NaHCO₃ solution (83 μL) was added to a mixture of DM4 (1.75 mg, 2.2 μmol, 1.0 eq.) and SPDB (0.73 mg, 2.2 μmol, 1.0 eq.) in DMA (0.4 mL) at rt. After stirring at rt for 10 min, the reaction mixture was added to L6Dap-thioester-NH₂ (5.0 mg, 2.2 μmol, 1.0 eq.) at rt. After stirring at rt for 15 min, TFA was added until acidic pH was reached. Purification on C18 (12 g, 20 to 80% of ACN+0.1% TFA in water+0.1% TFA over 12 CV) afforded L6Dap-thioester-SPDB-DM4 (1.4 mg, 0.5 μmol, UV purity 97%, 22% yield) as a white product after freeze-drying. UPLC-MS (method 4): Rt=3.06 min, m/z=1351 [M-2H]²⁻.

### Preparation of L6Orn-ester-NH₂

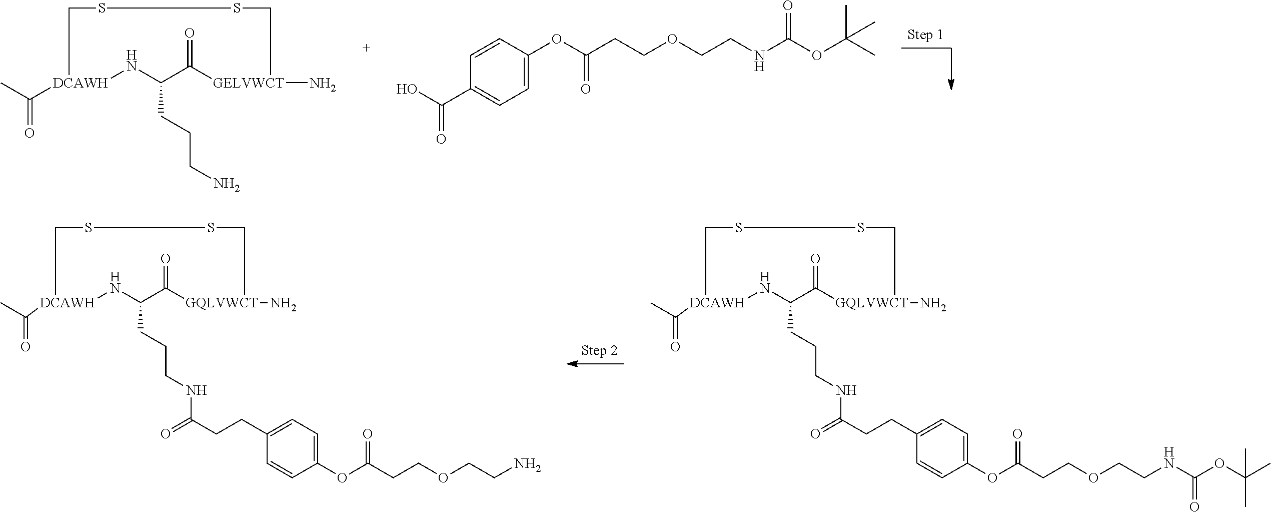

Step 1. HATU·HPF₆ (5.1 mg, 13.4 μmol, 1.1 eq.) followed by DIEA (5.1 μL, 29.3 μmol, 2.4 eq.) were added to a solution of 4-[3-[2-(tert-butoxycarbonylamino)ethoxy]propanoyloxy]benzoic acid (5.3 mg, 14.6 μmol, 1.2 eq.) in DMF (0.5 mL) at rt. After 5 min stirring at rt, L6Orn (19.2 mg, 12.2 μmol, 1.0 eq.) was added to the reaction mixture. After stirring at rt for 20 min, TFA was added until acidic pH was reached. Purification on C18 (12 g, 20 to 80% of ACN+0.1% TFA in water+0.1% TFA over 12 CV) afforded L6Orn-ester-NHBoc (21.5 mg, 7.3 μmol, UV purity 65%, 60% yield) as a white solid after freeze-drying. UPLC-MS (method 4): Rt=2.49 min, m/z=905 [M+2H-Boc]²⁺, 953 [M-2H]²⁻.

Step 2. TFA (1.0 mL) was added to a solution of L6Orn-ester-NHBoc (21.5 mg, UV purity 65%, 7.3 μmol, 1.0 eq.) at rt. The reaction mixture was stirred at rt for 10 min then concentrated in vacuo. Water (5 mL) and ACN (5 mL) were added, and then the mixture was freeze-dried to afford L6Orn-ester-NH₂ (23.5 mg, 6.8 μmol, UV purity 52%, 93% yield) as a white powder. UPLC-MS (method 4): Rt=1.80 min, m/z=905 [M+2H]²⁺, 903 [M-2H]²⁻.

### Preparation of L6Orn-ester-DOTA

DIEA (2.9 μL, 16.6 μmol, 6.6 eq.) was added to a solution of L6Orn-ester-NH₂ (5.0 mg, 91% purity, 2.5 μmol, 1.0 eq.) and DOTA-NHS ester·TFA·HPF₆ (2.1 mg, 2.8 μmol, 1.1 eq.) in DMF (0.2 mL) at rt. After stirring at rt for 2 h, TFA was added until acidic pH was reached. Purification on C18 (12 g, 20 to 80% of ACN+0.1% TFA in water+0.1% TFA over 12 CV) afforded L6Orn-ester-DOTA (4.2 mg, 1.2 μmol, UV purity 63%, 48% yield) as a white product after freeze-drying. UPLC-MS (method 4): Rt=1.80 min, m/z=1098 [M+2H]²⁺, 1096 [M-2H]²⁻.

### Preparation of L6Orn-ester-DTPA

DIEA (2.9 μL, 16.6 μmol, 6.6 eq.) was added to a solution of L6Orn-ester-NH₂ (5.0 mg, 91% purity, 2.5 μmol, 1.0 eq.) and p-SCN-Bn-CHX-A″-DTPA·3HCl (1.95 mg, 2.8 μmol, 1.1 eq.) in DMF (0.3 mL) at rt. After stirring at rt for 45 min, TFA was added until acidic pH was reached. Purification by reversed phase preparative HPLC (5 to 50% of ACN+0.1% FA in water+0.1% FA) afforded L6Orn-ester-DTPA (2.4 mg, 0.8 μmol, UV purity 78%, 32% yield) as a white product after freeze-drying. UPLC-MS (method 4): Rt=2.09 min, m/z=1202 [M+2H]²⁺, 1200 [M-2H]²⁻.

### Preparation of L6Orn-ester-PCTA

DIEA (2.9 μL, 16.6 μmol, 6.6 eq.) was added to a solution of L6Orn-ester-NH₂ (5.0 mg, 91% purity, 2.5 μmol, 1.0 eq.) and p-SCN-Bn-PCTA.3HCl (1.76 mg, 2.8 μmol, 1.1 eq.) in DMF (0.3 mL) at rt. After stirring at rt for 45 min, TFA was added until acidic pH was reached. Purification by reversed phase preparative HPLC (5 to 50% of ACN+0.1% FA in water+0.1% FA) afforded L6Orn-ester-PCTA (1.96 mg, 0.6 μmol, UV purity 73%, 24% yield) as a white product after freeze-drying. UPLC-MS (method 4): Rt=2.04 min, m/z=1169 [M+2H]²⁺, 1167 [M-2H]²⁻.

### Preparation of L6Orn-ester-Bn-NOTA

DIEA (1.6 μL, 9.6 μmol, 4.0 eq.) was added to a mixture of L6Orn-ester-NH₂ (5.0 mg, 2.4 μmol, 1.0 eq.) and p-SCN-Bn-NOTA (1.35 mg, 2.4 μmol, 1.0 eq.) in DMF (0.3 mL) at rt. After stirring at rt for 30 min, TFA was added until acidic pH was reached. Purification on C18 (12 g, 20 to 80% of ACN+0.1% TFA in water+0.1% TFA over 12 CV) afforded L6Dap-thioester-Bn-NOTA (2.9 mg, 1.2 μmol, UV purity 91%, 49% yield) as a white powder after freeze-drying. UPLC-MS (method 4): Rt=2.11 min, m/z=1130 [M+2H]²⁺, 1128 [M-2H]²⁻.

### Preparation of L6Orn-ester-FITC

DIEA (2.6 μL, 15.1 μmol, 6.0 eq.) was added to a solution of L6Orn-ester-NH₂ (5.0 mg, 91% purity, 2.5 μmol, 1.0 eq.) and 5-FITC (0.98 mg, 2.5 μmol, 1.0 eq.) in DMF (0.3 mL) at rt. After stirring at rt for 30 min, TFA was added until acidic pH was reached. Purification on C18 (12 g, 20 to 80% of ACN+0.1% TFA in water+0.1% TFA over 12 CV) afforded L6Orn-ester-FITC (3.3 mg, 1.5 μmol, UV purity 100%, 60% yield) as a white product after freeze-drying. UPLC-MS (method 4): Rt=2.49 min, m/z=1099 [M+2H]²⁺, 1097 [M-2H]²⁻.

### Preparation of L6Orn-ester-N₃

DIEA (2 μL, 11.7 μmol, 4.0 eq.) was added to a mixture of 2,5-dioxopyrrolidin-1-yl 2-azidoacetate (0.61 mg, 2.9 μmol, 1.0 eq.) and L6Orn-ester-NH₂ (8 mg, 2.9 μmol, 1.0 eq.) in DMF (0.3 mL) at rt. After stirring at rt for 35 min, TFA was added until acidic pH was reached. Purification by preparative HPLC (2 to 100% ACN+0.1% FA in water+0.1% FA) afforded L6Orn-ester-N₃ (4.00 mg, 1.7 mmol, 82% UV purity, 59% yield) as a white powder after freeze-drying. UPLC-MS (method 4): Rt=2.14 min, m/z=946 [M+2H]²⁺, 944 [M-2H]²⁻.

### Preparation of L6Orn-(CH₂)₂-ester-NH₂

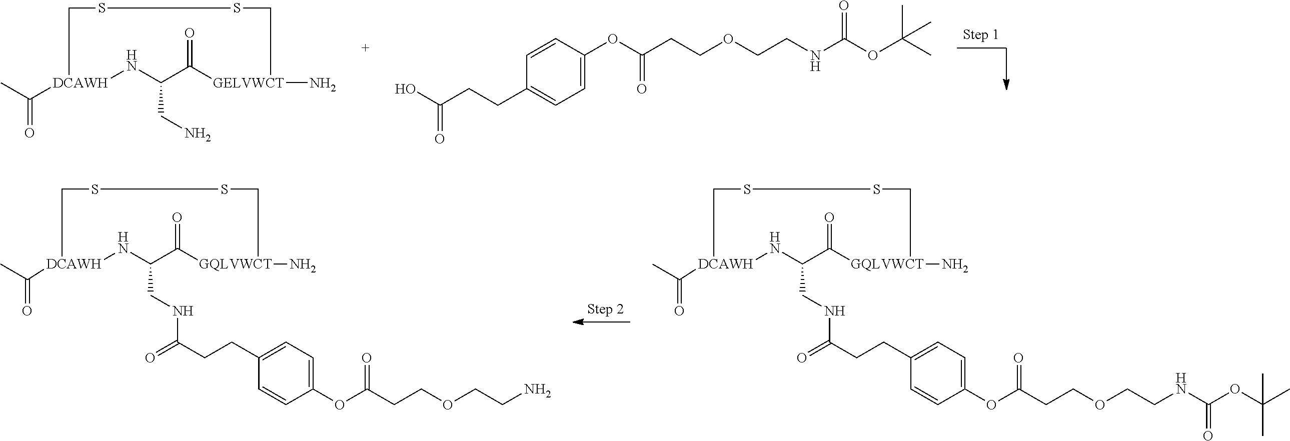

Step 1. HATU·HPF₆ (4.59 mg, 11.6 μmol, 1.2 eq.) followed by DIEA (4.1 μL, 23.3 μmol, 2.4 eq.) were added to a solution of 3-[4-[3-[2-(tert-butoxycarbonylamino)ethoxy]propanoyloxy]phenyl]propanoic acid (3.7 mg, 9.7 μmol, 1.0 eq.) in DMF (0.8 mL) at rt. After 5 min stirring at rt, L6Dap (15.0 mg, 9.7 μmol, 1.0 eq.) was added to the reaction mixture. After stirring at rt for 1.5 h, TFA was added until acidic pH was reached. Purification by reversed phase automated flash chromatography on a C18 biotage column (30 g, 20 to 80% of ACN+0.1% TFA in water+0.1% TFA over 12 CV) afforded L6Dap-(CH₂)₂-ester-NHBoc (11.4 mg, 6.0 μmol, UV purity 86%, 53% yield) as a white solid after freeze-drying. UPLC-MS (method 4): Rt=2.53 min, m/z=965 [M+2H+Na]²⁺, 952 [M-2H]²⁻.

Step 2. TFA (4.6 mL) was added to a solution of L6Dap-(CH₂)₂-ester-NHBoc (11.4 mg, 6.0 μmol, 1.0 eq.) at rt. The reaction mixture was stirred at rt for 10 min then concentrated in vacuo. Water (1 mL) and ACN (1 mL) were added, then the mixture was freeze-dried to afford L6Dap-(CH₂)₂-ester-NH₂ (13.5 mg, 5.9 μmol, UV purity 79%, 99% yield) as a white powder. UPLC-MS (method 4): Rt=1.82 min, m/z=904 [M+2H]²⁺, 903 [M-2H]²⁻.

### Preparation of L6Orn-(CH₂)₂-ester-DOTA

DIEA (2.2 μL, 12.8 μmol, 4.0 eq.) was added to a solution of L6Dap-(CH₂)₂-ester-NH₂ (5.8 mg, 3.2 μmol, 1.0 eq.) and DOTA-NHS ester·HPF₆·TFA (2.4 mg, 3.2 μmol, 1.0 eq.) in DMF (0.3 mL) at rt. The reaction mixture was stirred at rt for 1 h then two drops of TFA were added. Purification by preparative HPLC (5−50% ACN+0.1% FA in water+0.1% FA) afforded L6Dap-(CH₂)₂-ester-DOTA (4.0 mg, 1.8 μmol, UV purity 100%, 57% yield) as a white product after freeze-drying. UPLC-MS (method 4): Rt=2.82 min, m/z=732 [M+3H]³⁺, 1096 [M-2H]²⁻.

### Preparation of L6Orn-(CH₂)₂-ester-DTPA

DIEA (3.7 μL, 21.0 μmol, 6.0 eq.) was added to a solution of L6Dap-(CH₂)₂-ester-NH₂ (8.0. mg, 79% purity, 3.5 μmol, 1.0 eq.) and p-SCN-Bn-CHX-A″-DTPA·3HCl (2.46 mg, 3.5 μmol, 1.0 eq.) in DMF (0.2 mL) at rt. The reaction mixture was stirred at rt for 1.5 h then two drops of TFA were added. Purification by reversed phase preparative HPLC (5-50% ACN+0.1% FA in water+0.1% FA) afforded L6Dap-(CH₂)₂-ester-DTPA (5.9 mg, 2.1 μmol, UV purity 84%, 59% yield) as a white product after freeze-drying. UPLC-MS (method 4): Rt=2.82 min, m/z=801 [M+3H]³⁺, 1199 [M-2H]²⁻.

The MS characterization of peptide conjugates of Example 8 are shown in the table below. The MS data was obtained by ESI measured in a positive mode.

TABLE 12
Characterization of reactive conjugates

| | Cal- | | | | | |
|---|---|---|---|---|---|---|
| | culated | Cal- | | Retention | | UPLC- |
| | Exact | culated | Found | time | Purity | MS |
| Compound | Mass | m/z | m/z | (min) | (%) | method |
| A3K- | 2294.55 | 1148.28 | 1148 | 2.447 | 100 | 2 |
| thioester- | | | | | | |
| DOTA | | | | | | |
| L6Dap- | 2210.47 | 1106.24 | 1106 | 2.95 | 99 | 4 |
| thioester- | | | | | | |
| DOTA | | | | | | |
| L6Dap(- | 2209.47 | 1105.74 | 1105.6 | 2.277 | 100 | 2 |
| thioester- | | | | | | |
| DOTA)-E8Q | | | | | | |
| L6Dap- | 2418.7 | 1210.35 | 1210 | 2.3 | 80 | 4 |
| thioester- | | | | | | |
| DTPA | | | | | | |
| L6Dap- | 2351.66 | 1176.83 | 1176.4 | 2.514 | 90 | 2 |
| thioester- | | | | | | |
| PCTA | | | | | | |
| L6Dap- | 2345.76 | 1173.88 | 1173.3 | 2.954 | 95 | 2 |
| thioester- | | | | | | |
| Bn-NODA- | | | | | | |
| GA | | | | | | |
| L6Dap- | 2274.8 | 1138.4 | 1138 | 2.23 | 100 | 4 |
| thioester- | | | | | | |
| Bn-NOTA | | | | | | |
| L6Dap- | 2282.6 | 761.87 | 761 | 1.96 | 61 | 4 |
| thioester- | | | | | | |
| DOTA-GA | | | | | | |
| L6Dap- | 2755.11 | 919.37 | 919 | 2.46 | 99 | 4 |
| thioester- | | | | | | |
| triazole- | | | | | | |
| dbco-DFO | | | | | | |
| L6Dap- | 1907.12 | 956.56 | 954 | 2.30 | 92 | 4 |
| thioester-N₃ | | | | | | |
| L6Dap- | 2213.45 | 1107.73 | 1107 | 2.58 | 83 | 4 |
| thioester- | | | | | | |
| FITC | | | | | | |
| L6Dap- | 2781.65 | 1389.83* | 1389.6* | 3.056 | 92 | 2 |
| thioester- | | | | | | |
| MCC-DM1 | | | | | | |
| L6Dap- | 2704.7 | 1353.35 | 1353 | 3.06 | 97 | 4 |
| thioester- | | | | | | |
| SPDB-DM4 | | | | | | |
| L6Dab- | 2224.47 | 1113.24 | 1113 | 2.293 | 100 | 2 |
| thioester- | | | | | | |
| DOTA | | | | | | |
| L6Orn- | 2238.47 | 1120.24 | 1119.9 | 2.31 | 100 | 2 |
| thioester- | | | | | | |
| DOTA | | | | | | |
| L6K- | 2252.47 | 1127.24 | 1126.9 | 2.327 | 100 | 2 |
| thioester- | | | | | | |
| DOTA | | | | | | |
| L6Orn- | 2194.50 | 1098.25 | 1098 | 1.80 | 63 | 4 |
| ester-DOTA | | | | | | |
| L6Orn- | 2402.7 | 1202.35 | 1202 | 2.09 | 78 | 4 |
| ester-DTPA | | | | | | |
| L6Orn- | 2335.60 | 1168.80 | 1169 | 2.04 | 73 | 4 |
| ester-PCTA | | | | | | |
| L6Orn- | 2258.60 | 1130.30 | 1130 | 2.11 | 91 | 4 |
| ester-Bn- | | | | | | |
| NOTA | | | | | | |
| L6Orn- | 2197.4 | 1099.70 | 1099 | 2.49 | 100 | 4 |
| ester-FITC | | | | | | |
| L6Orn- | 1891.1 | 946.55 | 946 | 2.14 | 82 | 4 |
| ester-N₃ | | | | | | |
| L6Dap- | 2194.40 | 1098.20 | 1098 | 2.82 | 100 | 4 |
| (CH2)₂₋ | | | | | | |
| ester-DOTA | | | | | | |
| L6Dap- | 2402.63 | 1202.32 | 1202 | 2.475 | 97 | 2 |
| (CH2)₂₋ | | | | | | |
| ester-DTPA | | | | | | |

*peptide conjugate was measured in negative mode, [M − 2H]²⁻

### Example 9: Preparation and Characterization of Fc-Binding Vectors (Tyr Approach)

The Fc-binding vectors were prepared using standard Fmoc/tBu-based SPPS, including on-resin coupling and convergent strategies. The ligands prepared in Example 9 are shown in Table 13 below (bold-underlined indicates that a disulfide bond is present between the side chains of the respective Cys residues).

TABLE 13
Fc-binding vectors (Tyr approach)

| Compound | Structure |
|---|---|
| L6Y | Ac-DCAWHYGELVWCT-NH₂ |
| L6hY | Ac-DCAWHhYGELVWCT-NH₂ |
| E8hY | Ac-DCAWHLGhYLVWCT-NH₂ |

The ligands were prepared by the same protocol as in Example 1. The MS characterization of peptides of Example 9 is shown in the table below.

TABLE 14
Characterization of peptides (Tyr approach)

| | Cal- | Cal- | | | | |
|---|---|---|---|---|---|---|
| | culated | culated | Found | Retention | | UPLC- |
| | Exact | [M + | [M + | time | Purity | MS |
| Compound | Mass | 2H]²⁺ | 2H]²⁺ | (min) | (%) | method |
| L6Y | 1621.74 | 811.87 | 811.97 | 1.84 | 99 | 1 |
| L6hY | 1635.74 | 818.87 | 818.6 | 2.451 | 99 | 2 |
| E8hY | 2007.18 | 1004.59 | 1004.40 | 2.572 | 98 | 2 |

### Example 10: Preparation of DOTA-Containing Reactive Conjugates (Tyrosine Approach)

The Fc-binding vectors prepared in Example 9 were converted into reactive conjugates by coupling of a compound 010 to the hydroxyl group of the tyrosine or homo-tyrosine (hY) side chain of the respective Fc-binding vectors. Synthesis of compound L6Y-DOTA, taken as an example, is shown in a scheme below. The other DOTA-carbonate-peptide conjugates (Tyr approach) were prepared in a similar way. The structures of the DOTA-containing reactive conjugates prepared in Example 10 are shown in the table below.

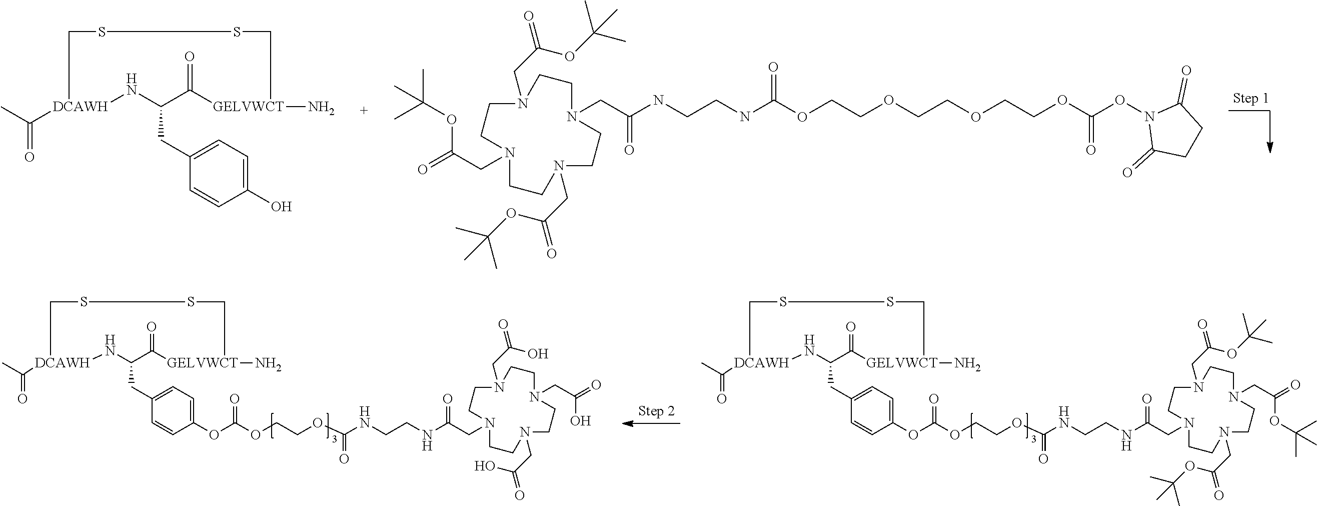

TABLE 15
Structures of DOTA-containing reactive conjugates (Tyr approach)

| Com- | |
|---|---|
| pound | Structure |
| L6Y- DOTA (L6Y + 010) | 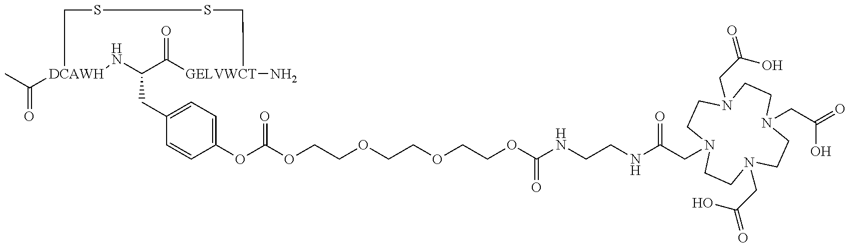 |
| L6hY- DOTA (L6hY + 010) | 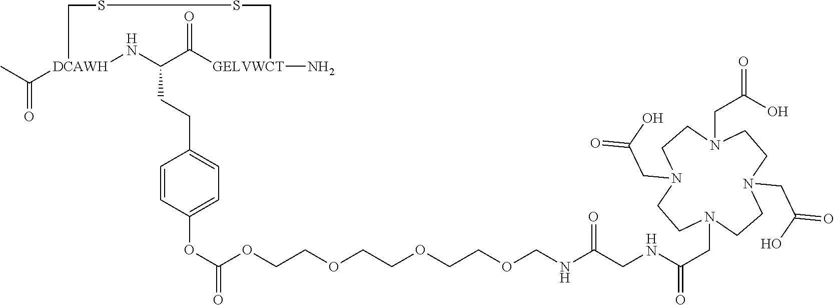 |
| E8hY- DOTA (E8hY + 010) | 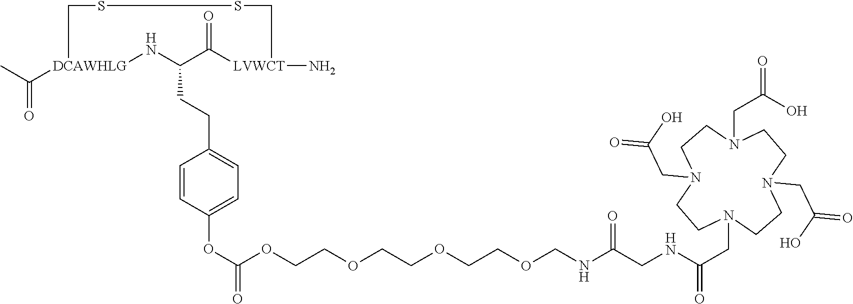 |

To prepare the reactive conjugates, 2 eq. of DMAP were added to a solution of compound 010 (2 eq.) in DMF and stirred for 1 min, followed by the addition of the Fc-binding vector (1 eq.) and DIEA (4 eq.). The reaction mixture was stirred for 1 to 4 hours at room temperature. Completion of the reaction was monitored by UPLC-MS. The reactive conjugate was precipitated with cold diethyl ether.

Subsequently, the tert-butyl protecting groups of the DOTA moiety were removed by treatment with TFA/TIS/water (95/2.5/2.5, v/v/v) over 1-3 hours at room temperature, followed by precipitation with cold diethyl ether and purification by HPLC (as described in Example 6).

The MS characterization of DOTA-peptide conjugates is shown in the table below.

TABLE 16
Characterization of DOTA-reactive conjugates

| | Cal- | Cal- | | | | |
|---|---|---|---|---|---|---|
| | culated | culated | Found | Retention | | UPLC- |
| | Exact | [M + | [M + | time | Purity | MS |
| Compound | Mass | 2H]²⁺ | 2H]²⁺ | (min) | (%) | method |
| L6Y-DOTA | 2269.74 | 1135.87 | 1136.12 | 1.58 | 54 | 1 |
| L6hY-DOTA | 2283.74 | 1142.87 | 1142.97 | 1.6 | 60 | 1 |
| E8hY-DOTA | 2655.18 | 1328.59 | 1328.14 | 1.72 | 80 | 1 |

### Example 11: Preparation of Trastuzumab-Payload Conjugates

The propensity of the reactive conjugates of Examples 5-8, 10 to react with an antibody was evaluated using trastuzumab as a model system.

To prepare the trastuzumab-payload conjugates, 2 eq of reactive conjugate prepared in Examples 5-8, 10 (2.4 nmol) in DMSO (2-7% v/v in final reaction mixture) was added to a solution of trastuzumab (1 eq, 1.2 nmol; commercial trastuzumab Herceptin® available from Roche which was buffer-exchanged into phosphate-buffered saline (PBS) prior to the conjugation) diluted in 50 mM NaHCO₃ pH 9.0 (20-28 μL) and the reaction mixture (36 μL) was stirred at room temperature for 2 hours.

After payload conjugation, the reaction buffer was diluted with 64 μL of 0.1 M glycine pH 2.5. The antibody conjugate was then purified by gel filtration chromatography using a pre-equilibrated with 0.1 M glycine pH 2.5 Bio-spin P-30 Column (bed height: 3.7 cm, overall length: 5 cm; available from Bio-Rad, USA) and then eluted with 0.1M glycine pH 2.5. The purified antibody conjugate fractions were neutralized with 1M PBS pH 8.5 (10 μL).

The conjugation of the payload moiety to trastuzumab was evaluated by HRMS analysis (as described above). The payload loading ratios (selectivity) between Fc and F(ab)₂ were evaluated by digesting the conjugates with GingisKhan protease (1 unit per μg of antibody conjugate in the presence of 2 mM cysteine, 0.1M Tris, pH 8.0 for 1 hour at 37° C.), and subsequent HRMS analysis (as described above). The Degree of Conjugation (DoC) of the trastuzumab-payload conjugates was evaluated based on the results of the HRMS analysis (as described above).

The results of the HRMS analysis are shown in Table 17 below.

TABLE 17
Characterization of trastuzumab-payload
conjugates prepared in Example 11

| | | Labeled | DoC | DoC | Selectivity |
|---|---|---|---|---|---|
| Compound | DoC | mAb (%) | Fc | F(ab)₂ | Fc/F(ab)₂ |
| D1K-carbonate- | 0.52 | 42.1 | 0.24 | 0.26 | 0.9 |
| DOTA | | | | | |
| A3K-carbonate- | 1.33 | 85.3 | 1.19 | 0.12 | 10.2 |
| DOTA | | | | | |
| W4K-carbonate- | 0.23 | 21.3 | 0.05 | 0.26 | 0.2 |
| DOTA | | | | | |
| H5K-carbonate- | 0.37 | 32.7 | 0.34 | 0.10 | 3.5 |
| DOTA | | | | | |
| L6K-carbonate- | 1.24 | 82.0 | 1.15 | 0.08 | 14.9 |
| DOTA | | | | | |
| G7K-carbonate- | 0.62 | 47.8 | 0.12 | 0.28 | 0.4 |
| DOTA | | | | | |
| E8K-carbonate- | 1.66 | 89.2 | 1.07 | 0.25 | 4.3 |
| DOTA | | | | | |
| L9K-carbonate- | 0.30 | 27.1 | 0.06 | 0.11 | 0.5 |
| DOTA | | | | | |
| V10K-carbonate- | 0.20 | 18.5 | 0.12 | 0.16 | 0.7 |
| DOTA | | | | | |
| T13K-carbonate- | 0.21 | 19.1 | 0.06 | 0.16 | 0.4 |
| DOTA | | | | | |
| A3Dap- | 0.29 | 25.5 | 0.04 | 0.18 | 0.19 |
| carbonate-DOTA | | | | | |
| A3Orn- | 0.64 | 46.6 | 0.37 | 0.04 | 8.79 |
| carbonate-DOTA | | | | | |
| A3hK-carbonate- | 1.54 | 92.4 | 1.50 | 0.03 | 46.26 |
| DOTA | | | | | |
| A3K(-carbonate- | 1.69 | 87.2 | 1.33 | 0.27 | 5.0 |
| DOTA)-L6Orn(- | | | | | |
| carbonate- | | | | | |
| DOTA) | | | | | |
| A3Dap-PEG₁- | 0.63 | 46.0 | 0.24 | 0.05 | 4.77 |
| carbonate-DOTA | | | | | |
| H5Dap(- | 0.66 | 50.1 | 0.38 | 0.27 | 1.4 |
| carbonate- | | | | | |
| DOTA)-E8Q | | | | | |
| H5Dab- | 0.61 | 48.8 | 0.43 | 0.15 | 2.9 |
| carbonate-DOTA | | | | | |
| H5Dab(- | 0.90 | 64.8 | 0.60 | 0.22 | 2.8 |
| carbonate- | | | | | |
| DOTA)-E8Q | | | | | |
| H5Dab-FITC | 0.94 | 63.8 | 0.13 | 0.29 | 0.45 |
| H5Orn(- | 1.04 | 69.9 | 0.76 | 0.21 | 3.6 |
| carbonate- | | | | | |
| DOTA)-E8Q | | | | | |
| H5K(-carbonate- | 1.04 | 71.2 | 0.74 | 0.21 | 3.5 |
| DOTA)-E8Q | | | | | |
| H5K-FITC | 0.71 | 52.0 | 0.07 | 0.17 | 0.42 |
| L6Dap- | 1.70 | 95.6 | 1.71 | 0.11 | 15.3 |
| carbonate-DOTA | | | | | |
| L6(D-Dap-DOTA) | 0.53 | 38.9 | 0.07 | 0.34 | 0.20 |
| L6Dap-(CH₂)₂- | 0.22 | 20.0 | 0.27 | 0.00 | Fully |
| carbonate-DOTA | | | | | selective |
| L6Dap(- | 1.79 | 97.9 | 1.79 | 0.10 | 17.9 |
| carbonate- | | | | | |
| DOTA)-E8Q | | | | | |
| L6Dab- | 1.11 | 76.0 | 1.03 | 0.14 | 7.5 |
| carbonate-DOTA | | | | | |
| L6Orn- | 1.87 | 99.0 | 1.93 | 0.05 | 41.5 |
| carbonate-DOTA | | | | | |
| L6(D-Orn- | 1.25 | 79.3 | 1.13 | 0.07 | 16.82 |
| carbonate- | | | | | |
| DOTA) | | | | | |
| L6(D-Orn- | 0.26 | 24.4 | 0.04 | 0.18 | 0.19 |
| carbonate- | | | | | |
| DOTA)^{#} | | | | | |
| L6Orn(- | 1.70 | 95.1 | 1.66 | 0.07 | 22.7 |
| carbonate- | | | | | |
| DOTA)-E8Q | | | | | |
| L6Orn(- | 1.53 | 92.5 | 1.52 | 0.06 | 24.6 |
| carbonate- | | | | | |
| DOTA)-E8Q* | | | | | |
| L6Orn- | 2.05 | 99.2 | 1.72 | 0.05 | 34.4 |
| carbonate-DTPA | | | | | |
| L6Orn- | 1.86 | 92.4 | 1.38 | 0.12 | 11.63 |
| carbonate-PCTA | | | | | |
| L6Orn- | 1.81 | 97 | 1.77 | 0 | Fully |
| carbonate- | | | | | selective |
| NODA-GA | | | | | |
| L6Orn- | 0.31 | 27.8 | 0.20 | 0.11 | 1.90 |
| carbonate-DFO | | | | | |
| L6Orn- | 2.31 | 96.6 | 2.21 | 0.10 | 22.1 |
| carbonate-FITC | | | | | |
| L6Orn- | 1.87 | 93.7 | 0.97 | 0.05 | 20.6 |
| carbonate-FAM | | | | | |
| L6Orn- | 0.93 | 70 | 0.45 | 0 | Fully |
| carbonate-TZ | | | | | selective |
| L6Orn- | 2.93 | 99 | 2.81 | 0.12 | 23.3 |
| carbonate-TCO | | | | | |
| L6Orn- | 2.20 | 99 | 2.02 | 0 | Fully |
| carbonate-N₃ | | | | | selective |
| L6Orn- | 1.65 | 87 | 1.56 | 0.08 | 19.46 |
| carbonate-DBCO | | | | | |
| L6Orn- | 1.97 | 92 | 1.94 | 0.03 | 64.59 |
| carbonate-BCN | | | | | |
| L6Orn- | 1.78 | 92 | 1.43 | 0 | Fully |
| carbonate- | | | | | selective |
| maleimide | | | | | |
| L6Orn- | 1.12 | 73 | 0.44 | 0.08 | 5.7 |
| carbonate-MCC- | | | | | |
| DM1 | | | | | |
| L6Orn- | 1.05 | 68 | 0.99 | 0.06 | 16.2 |
| carbonate-SPDB- | | | | | |
| DM4 | | | | | |
| L6Orn- | 1.01 | 71 | 0.94 | 0.07 | 13.43 |
| carbonate- | | | | | |
| Vedotin | | | | | |
| L6Orn- | 0.06 | 94 | 0.06 | 0 | Fully |
| carbonate- | | | | | selective |
| (Vedotin)₂ | | | | | |
| L6Orn- | 1.12 | 73 | 1.04 | 0.08 | 13.5 |
| carbonate-multi- | | | | | |
| DM1 | | | | | |
| L6Orn- | 0.46 | 39.9 | 0.16 | 0.00 | Fully |
| carbonate-multi- | | | | | selective |
| (DM1)₂ | | | | | |
| L6K-carbonate- | 1.20 | 78.0 | 0.49 | 0.09 | 5.68 |
| FITC | | | | | |
| L6K-PEG₁- | 2.22 | 98.2 | 1.74 | 0.05 | 33.3 |
| carbonate-DOTA | | | | | |
| L6K-PEG₂- | 1.70 | 94.6 | 1.95 | 0.05 | 36.0 |
| carbonate-DOTA | | | | | |
| L6K-PEG₃- | 2.28 | 100.0 | 2.12 | 0.14 | 15.3 |
| carbonate-DOTA | | | | | |
| L6K-PEG₁₀- | 1.56 | 90.3 | 1.47 | 0.16 | 9.3 |
| carbonate-DOTA | | | | | |
| A3K-thioester- | 1.31 | 91 | 1.34 | 0 | Fully |
| DOTA | | | | | selective |
| L6Dap-thioester- | 1.63 | 94.1 | 1.52 | 0.04 | 36.2 |
| DOTA | | | | | |
| L6Dap(-thioester- | 1.50 | 92 | 1.35 | 0 | Fully |
| DOTA)-E8Q | | | | | selective |
| L6Dap-thioester- | 0.18 | 18 | 0.03 | 0 | Fully |
| DTPA | | | | | selective |
| L6Dap-thioester- | 0.89 | 63 | 0.60 | 0.21 | 2.80 |
| PCTA | | | | | |
| L6Dap-thioester- | 0.10 | 10 | 0.16 | 0 | Fully |
| MCC-DM1 | | | | | selective |
| L6Dab-thioester- | 0.70 | 55 | 0.34 | 0.17 | 2.03 |
| DOTA | | | | | |
| L6Orn-thioester- | 1.48 | 94 | 1.39 | 0 | Fully |
| DOTA | | | | | selective |
| L6K-thioester- | 1.37 | 88 | 1.21 | 0.05 | 24.49 |
| DOTA | | | | | |
| L6Dap-(CH₂)₂- | 0.10 | 9.6 | 0.05 | 0.00 | Fully |
| ester-DOTA | | | | | selective |
| L6Orn-ester-Bn- | 1.25 | 78 | 0.90 | 0 | Fully |
| NOTA | | | | | selective |
| L6Y-DOTA | 1.42 | 92.3 | 1.56 | 0.0 | Fully |
| | | | | | selective |
| L6hY-DOTA | 0.32 | 29.6 | 0.33 | 0.02 | 14.9 |
| E8Dap- | 2.09 | 97.0 | 1.69 | 0.28 | 5.97 |
| carbonate-DOTA | | | | | |
| E8Orn- | 2.47 | 100.0 | 2.09 | 0.00 | Fully |
| carbonate-DOTA | | | | | selective |
| E8hY-DOTA | 0.73 | 59.8 | 0.78 | 0.00 | Fully |
| | | | | | selective |
| L6Orn- | 1.42 | 92 | 1.29 | 0 | Fully |
| carbonate-PEG₅- | | | | | selective |
| Bn-NOTA | | | | | |
| A3hK(- | 2.20 | 93 | 2.06 | 0.19 | 11.0 |
| carbonate- | | | | | |
| DOTA)-E8Orn(- | | | | | |
| carbonate- | | | | | |
| DOTA) | | | | | |
| L6Orn- | 1.44 | 83 | 1.54 | 0 | Fully |
| carbonate-Bn- | | | | | selective |
| TCMC | | | | | |
| L6Orn-ester-N₃ | 1.71 | 86 | 1.11 | 0 | Fully |
| | | | | | selective |
| A3hK(- | 1.23 | 75 | 1.15 | 0.04 | 31.0 |
| carbonate-N₃)- | | | | | |
| E8Orn(- | | | | | |
| carbonate-N₃) | | | | | |
| Alexa647-NHS | 4.40 | 100 | 2.18 | 2.22 | 1.0 |
| (random | | | | | |
| conjugation)^{a} | | | | | |
| L6Orn- | 1.59 | 92 | 1.17 | 0 | Fully |
| carbonate- | | | | | selective |
| Alexa647^{b} | | | | | |
| Carbonate-FITC | 0.83 | 59 | 0.29 | 0.54 | 0.5 |
| (random | | | | | |
| conjugation)^{c} | | | | | |
| L6Orn- | 0.90 | 67 | 1.08 | 0 | Fully |
| carbonate- | | | | | selective |
| diamino-(PEG₃₇)₂ | | | | | |
| L6Orn- | 1.52 | 89 | 1.16 | 0.03 | 36.4 |
| carbonate-PEG₃₇ | | | | | |
| L6Orn- | 1.22 | 77 | 1.15 | 0.07 | 17.5 |
| carbonate-PEG₅- | | | | | |
| PDB-DM4 | | | | | |
| L6Orn(- | 1.24 | 75 | 1.18 | 0.06 | 18.1 |
| carbonate-PDB- | | | | | |
| DM4)-PEG₂₄ | | | | | |
| L6Orn(- | 1.28 | 80 | 1.22 | 0.06 | 19.8 |
| carbonate- | | | | | |
| Vedotin)-PEG₂₄- | | | | | |
| G | | | | | |
| PEG₂₀-L6Orn- | 1.27 | 78 | 1.19 | 0.08 | 14.0 |
| carbonate-PDB- | | | | | |
| DM4 | | | | | |
| PEG₂₀-L6Orn- | 1.30 | 80 | 1.30 | 0 | Fully |
| carbonate- | | | | | selective |
| Vedotin | | | | | |
| PEG₂₀-L6Orn- | 1.63 | 89 | 1.54 | 0.07 | 21.2 |
| carbonate- | | | | | |
| Vedotin^{d} | | | | | |
| PEG₂₄-L6Orn- | 1.54 | 95 | 1.49 | 0.05 | 31.8 |
| carbonate-PEG₃- | | | | | |
| (suc-VC-PAB- | | | | | |
| MMAE) | | | | | |
| PEG₂₄-L6Orn- | 1.12 | 74 | 1.07 | 0.05 | 21.7 |
| carbonate-PEG₃- | | | | | |
| DM4 | | | | | |
| PEG₂₄-L6Orn- | 1.90 | 96 | 1.82 | 0.08 | 23.0 |
| carbonate-PEG₃-- | | | | | |
| (suc-VC-PAB- | | | | | |
| MMAE) ^{e} | | | | | |
| PEG₂₄-L6Orn- | 1.49 | 78 | 1.41 | 0.08 | 18.0 |
| carbonate-PEG₃- | | | | | |
| DM4^{e} | | | | | |
| L6Orn- | 1.87 | 98 | 1.84 | 0.03 | 73.2 |
| carbonate-VC- | | | | | |
| PAB-MMAE | | | | | |
| PEG₂₄-L6Orn- | 1.81 | 96 | 1.79 | 0.02 | 76.4 |
| carbonate-PEG1- | | | | | |
| VC-PAB-MMAE | | | | | |
| PEG₂₄-L6Orn- | 1.25 | 57 | 1.23 | 0.02 | 57.7 |
| carbonate-PNU | | | | | |
| L6Orn- | 1.05 | 46 | 1.01 | 0.04 | 24.4 |
| carbonate-PNU | | | | | |
| L6Orn- | 0.49 | 36 | 0.49 | 0 | Fully |
| carbonate-multi- | | | | | selective |
| (DM1)-K(PEG₂₄)- | | | | | |
| (DM1) | | | | | |
| L6Orn- | 0.68 | 52 | 0.53 | 0 | Fully |
| carbonate-multi- | | | | | selective |
| (DM1)₂-PEG₁₅ | | | | | |
| PEG₂₀-L6Orn- | 1.11 | 75 | 1.14 | 0 | Fully |
| carbonate-multi- | | | | | selective |
| (DM1)₂^{f} | | | | | |
| PEG₂₀-L6Orn- | 1.27 | 77 | 1.34 | 0.04 | 30.6 |
| carbonate-multi- | | | | | |
| (DM1)₂^{g} | | | | | |
| RGNCAYHK(NH | 0 | 0 | | | ND |
| S-N₃-DBCO- | | | | | |
| MMAE)GQIIWCT | | | | | |
| YH | | | | | |
| RGNCAYHK(NH | 0.10 | 10 | | | ND |
| S-PEG₁- | | | | | |
| MMAE)GQIIWCT | | | | | |
| YH | | | | | |
| RGNCAYHK(carbonate- | 0 | 0 | | | ND |
| N₃-DBCO- | | | | | |
| MMAE)GQIIWCT | | | | | |
| YH | | | | | |
| RGNCAYHK(NH | 0.03 | 3 | | | ND |
| S-N₃-DBCO- | | | | | |
| MMAE)GQIIWCT | | | | | |
| YH^{h} | | | | | |
| RGNCAYHOrn(N | 0.02 | 1 | | | ND |
| HS-N₃-DBCO- | | | | | |
| MMAE)GQIIWCT | | | | | |
| YH^{h} | | | | | |
| RGNCAYHK(NH | 0.56 | 44 | 0.55 | 0.01 | 55.0 |
| S-PEG₁- | | | | | |
| MMAE)GQIIWCT | | | | | |
| YH^{h} | | | | | |
| RGNCAYHK(carbonate- | 0 | 0 | | | ND |
| N₃-DBCO- | | | | | |
| MMAE)GQIIWCT | | | | | |
| YH^{h} | | | | | |
| RGNCAYHK(carbonate- | 0.01 | 1 | 0 | 0 | Fully |
| suc-VC-PAB- | | | | | selective |
| MMAE)GQIIWCT | | | | | |
| YH | | | | | |

#peptide sequence consists of D amino acids;
*trastuzumab with excipients;
ND—not determined;
^{a}9 eq of peptide-payload conjugate,
^{b}2.3 eq of peptide-payload conjugate,
^{c}10 eq of peptide-payload conjugate,
^{d}2.8 eq of peptide-payload conjugate,
^{e}2.4 eq of peptide-payload conjugate,
^{f}3 eq of peptide-payload conjugate,
^{g}4 eq of peptide-payload conjugate,
^{h}20 eq of peptide-payload conjugate (antibody conjugation was done in 50 mM Hepes buffer pH 7.2 during 3 h)

The antibody digestion with GingisKHAN™ or FabRICATOR™ enzymes often caused a cleavage of payload from Fc subunit, leading to a lower DoC. In these cases, the DoC of Fc were extrapolated (values in bold):

DoC Fc=DoC−DoC F(ab)₂, Selectivity Fc/F(ab)₂=(DoC−DoC F(ab)₂)/Doc (Fab)₂

These results indicate that peptide reactive conjugates could produce trastuzumab-payload conjugates with excellent selectivities, e.g. full selectivity, for the Fc region of the antibody.

The trastuzumab-payload conjugates were cleaved by specific enzyme and analyzed by tandem MS/MS analysis to determine the conjugation sites of the payload in the Fc fragment of trastuzumab. The results are shown in Table 18 below.

TABLE 18
Conjugation sites of the payload on the trastuzumab
determined by peptide mapping (lysines marked in
bold are the major labeling sites of trastuzumab).

| | | Labeled lysine |
|---|---|---|
| Compound | Enzyme | (EU numbering) |
| A3K-carbonate-DOTA | Fabricator | K392, K409/414, |
| | | K439 |
| H5Dab-carbonate-DOTA | GingisKhan + | K248/246* |
| | TCEP | |
| H5K-carbonate-DOTA | GingisKhan + | K248/246* |
| | TCEP | |
| L6Dap-carbonate-DOTA | Fabricator | K248/246*, K274 |
| L6Dap(-carbonate-DOTA)- | Fabricator | K248/246*, K274 |
| E8Q | | |
| L6Dab-carbonate-DOTA | Fabricator | K274, K248/246* |
| L6Orn-carbonate-DOTA | Fabricator | K248/246*, K274 |
| L6Orn-carbonate-DOTA- | Fabricator | K248/246*, K274 |
| Indium | | |
| L6Orn(carbonate-DOTA)- | Fabricator | K248, K246, |
| E8Q | | K274 |
| L6Orn-carbonate-DTPA | Fabricator | K274, K248/246* |
| L6Orn-carbonate-DTPA- | Fabricator | K248, K274 |
| Indium | | |
| L6Orn-carbonate-FITC | Fabricator | K248/246*, K274 |
| L6K-carbonate-DOTA | GingisKhan + | K392, K274 |
| | TCEP | |
| L6K-PEG₁-carbonate- | Fabricator | K246, K248, K274 |
| DOTA | | |
| L6K-PEG₂-carbonate- | Fabricator | K317, K326 |
| DOTA | | |
| L6Y-carbonate-DOTA | Fabricator | K246, K248, K274 |
| E8hY-carbonate-DOTA | Fabricator | K246, K248, K274 |

The lysine of the Fc region marked in bold seems to be almost quantitatively labeled, while labeling of other lysine was additionally observed in conjugates with higher DoCs bearing 3 payload moieties per Fc region.

*—It was found that, in most conjugates, the lysines 246 and 248 appeared in the same peptide fragment. According to the crystal structure information (DeLano et al. Science 2000, 287, 1279-1283), the distance between Fc-III L6 and Fc K248 (5.9 Å) was shorter than that between Fc-III L6 and K246 (17.3 Å), leading to a conclusion that the majority of the modification seems to be located at K248 but labeling of K246 cannot be excluded (K248/246).

The variation of the lysine position mutation or spacer length of L6 mutant led to the modification of different lysines in the Fc region: K392, K248, K246, K274, K317.

### Example 12: Preparation of Antibody-DOTA/FITC Conjugates Using Other Antibodies

The propensity of the reactive conjugates of the present invention to react with different antibodies was evaluated using commercially available atezolizumab, rituximab, trastuzumab emtansine, brentuximab-vedotin, aflibercept, panitumumab, pembrolizumab and Fc region only. Antibody-DOTA/FITC conjugates were prepared according to the same procedure as described in Example 11 above using peptide conjugates (indicated in Table 19) and the aforementioned antibodies.

The results of the HRMS analysis are shown in Table 19 below.

TABLE 19
Characterization of antibody-payload conjugates prepared in Example 12

| | | | Labeled mAb | | | Selectivity |
|---|---|---|---|---|---|---|
| Compound | Antibody | DoC | (%) | DoC Fc | DoC F(ab)₂ | Fc/F(ab)₂ |
| L6Orn-carbonate- | Atezolizumab | 2.12 | 100 | 2.06 | 0.06 | 34.3 |
| DOTA | | | | | | |
| L6Orn-carbonate-Bn- | Atezolizumab | 1.80 | 94 | 1.19 | 0.07 | 17.1 |
| NOTA | | | | | | |
| L6Orn-ester-Bn-NOTA | Atezolizumab | 0.96 | 68 | 0.88 | 0.08 | 10.5 |
| L6Orn-carbonate-PCTA | Atezolizumab | 1.70 | 94 | 1.45 | 0.03 | 43.6 |
| L6Orn-carbonate- | Atezolizumab | 1.36 | 84 | 1.05 | 0.06 | 18.2 |
| NOTA*ⁱ | | | | | | |
| L6Orn-carbonate- | Atezolizumab | 1.24 | 80 | 0.81 | 0.05 | 16.8 |
| NOTAⁱ | | | | | | |
| L6Orn-carbonate- | Atezolizumab | 1.76 | 97 | 1.60 | 0.02 | 79.7 |
| NODAGA | | | | | | |
| L6Orn-carbonate-Bn- | Atezolizumab | 1.66 | 94 | 1.32 | 0.06 | 23.1 |
| NODAGA | | | | | | |
| L6Orn-carbonate- | Atezolizumab | 1.55 | 95 | 1.44 | 0.03 | 41.4 |
| PEG₅-Bn-NOTA | | | | | | |
| carbonate-PEG₅-Bn- | Atezolizumab | 0.41 | 36 | 0.29 | 0.12 | 2.4 |
| NOTA^{j} | | | | | | |
| L6Orn-carbonate- | Rituximab | 1.67 | 96 | 1.48 | 0.00 | Fully selective |
| DOTA | | | | | | |
| L6Orn-carbonate- | Rituximab | 1.51 | 91 | 1.45 | 0.06 | 23.3 |
| Alexa647 | | | | | | |
| Alexa647-NHS | Rituximab | 4.65 | 100 | 1.86 | 2.79 | 0.7 |
| (random conjugation)^{a} | | | | | | |
| L6Orn-carbonate-FITC | Rituximab | 2.32 | 99 | 2.19 | 0.13 | 17.4 |
| Carbonate-FITC | Rituximab | 0.84 | 59 | 0.64 | 0.20 | 3.2 |
| (random conjugation)^{c} | | | | | | |
| L6Orn-carbonate- | Fc region only | — | 100 | 1.90 | — | — |
| DOTA | | | | | | |
| L6Orn-carbonate-FITC | Fc region only | — | 100 | 2.23 | — | — |
| L6Orn-carbonate-FITC | Trastuzumab | 1.66 | 95 | 1.60 | 0.11 | 14.8 |
| | emtansine | | | | | |
| L6Orn-carbonate- | Aflibercept | ND | ND | 1.52 | ND | ND |
| DOTA | | | | | | |
| L6Orn-carbonate- | Panitumumab | 2.13 | 99 | 1.92 | 0.01 | 158.6 |
| DOTA | | | | | | |
| L6Orn-carbonate- | Pembrolizumab | 1.90 | 97 | 1.85 | 0.02 | 85.4 |
| DOTA | | | | | | |

*atezolizumab with excipients;
ⁱ1.6 eq of peptide-payload conjugate,
^{h}-0 eq of peptide-payload conjugate (antibody conjugation was done during 4 h)

The antibody digestion with GingisKHAN™ or FabRICATOR™ enzymes often caused a cleavage of payload from Fc subunit, leading to a lower DoC. In these cases, the DoC of Fc were extrapolated (values in bold):

DoC Fc=DoC−DoC F(ab)₂, Selectivity Fc/F(ab)₂=(DoC−DoC F(ab)₂)/Doc (Fab)₂

In some cases, it was not possible to determine the labeling selectivity in case of atezolizumab due to poor stability of its Fc-payload fragment under enzyme treatment conditions.

It was found that compounds L6Orn-carbonate-DOTA, L6Orn-carbonate-FITC, L6Orn-carbonate-NOTA, L6Orn-carbonate-NODAGA led to efficient Fc region labeling with DoC comparable to trastuzumab.

### Example 13: Preparation of Immobilized FITC/DOTA-Containing Reactive Conjugate and Solid-Phase Modification of Trastuzumab

A reactive conjugate immobilized on a solid support was prepared, and its propensity to react with trastuzumab was evaluated.

TABLE 20
Structures of peptide-containing azide or biotin for solid support
immobilization.

| Compound | Structure |
|---|---|
| Biotin- L6Orn | 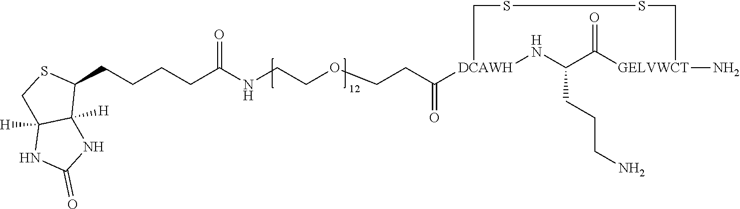 |
| L6Orn (—H)—K (-biotin) | 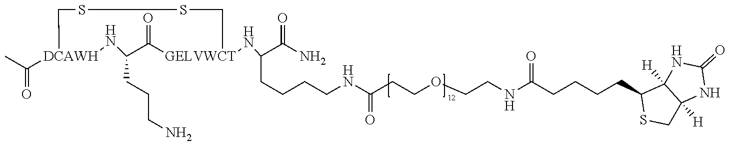 |
| N₃-L6Orn | 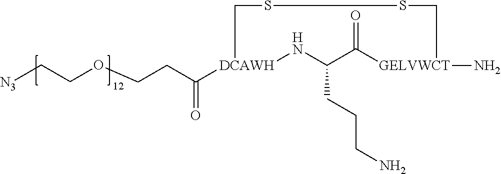 |
| L6Orn (—H)—N₃ | 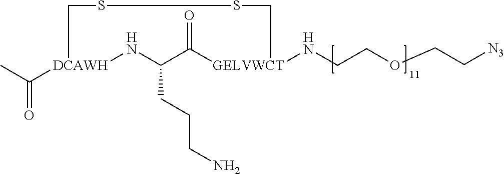 |
| N₃-L6K | 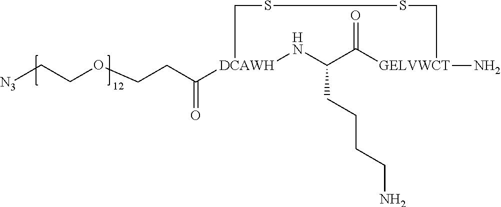 |

### Preparation of Compounds Peptides for Solid Support Immobilization

The peptides were prepared by standard Fmoc/tBu-based SPPS using a Rink Amide AM resin (loading: 0.57 mmol/g) and a Liberty Blue™ automated microwave peptide synthesizer (available from CEM Corp., Germany).

Coupling reactions for amide bond formation were performed over 4 min at room temperature using 0.2 M of Fmoc-amino-acids pre-activated with 0.5 M DIC and 1 M OxymaPure® in DMF. Fmoc deprotection was performed with 10% piperazine in DMF (v/v).

After completion of the synthesis, the resin (0.2 mmol) was washed with DMF and DCM. Then, the resin of L6Orn, L6Orn(-H)-K and L6K was swollen in DMF (14 mL). The azide-PEG₁₂-NHS or biotin-PEG₁₂-NHS (1 eq) was added to the resin, followed by DIEA (1 eq) and stirred for 72 hours at room temperature. The resin was washed with DMF and DCM.

The peptides were cleaved from the resin manually under gentle agitation over 1.5 hour at room temperature by treatment with TFA/TIS/water (90/5/5, v/v/v). After filtration and evaporation of the cleavage mixture with a nitrogen stream, the crude peptides were precipitated with cold diethyl ether, centrifuged, washed with cold diethyl ether and dried.

For disulfide bond formation, the crude peptides (0.1 mmol) were resuspended in 10 m1 DMSO, then 2 eq of 2 M NH₃ in MeOH and 50 eq of hydrogen peroxide were added and stirred at room temperature for 30 min. The progress of the oxidation was monitored via analytical UPLC-MS. 10 ml of 0.1% TFA acid aqueous solution was added to the solution to stop the reaction. The peptides were isolated after HPLC purification (as described in Example 1).

### Preparation of L6Orn(-H)-N₃)

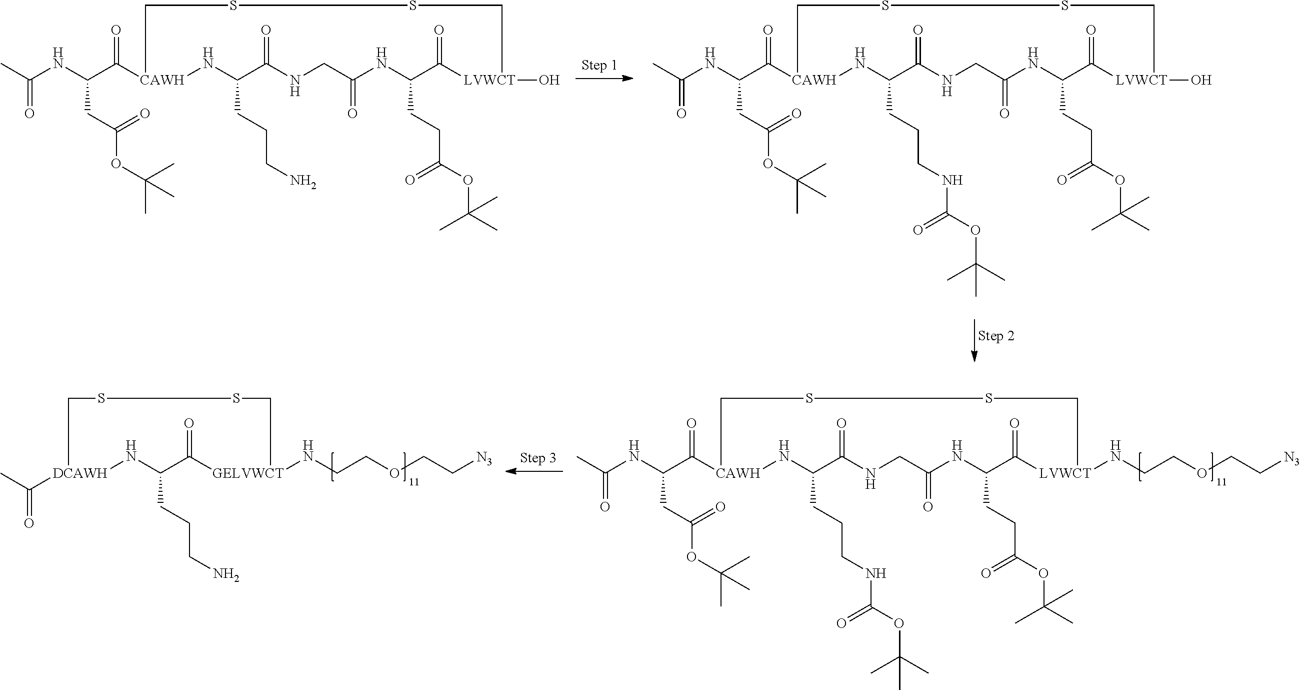

Step 1. Boc₂O (5.4 mg, 24.7 μmol, 1.1 eq.) and TEA (18.3 μL, 134.9 μmol, 6.0 eq.) were added to a solution of H-Fc-III—(OtBu)₂-L6Orn-OH (39.1 mg, 22.5 μmol, 1.0 eq.) in DMF (0.23 mL) at rt. After stirring at rt for 3 h, cold diethylether was added to the reaction mixture to precipitated the peptide. After centrifugation, the supernatant was removed, and the crude product was used in the next step assuming a quantitative yield. UPLC-MS (method 4): Rt=2.96 min, m/z=1787 [M+H]*, 1785 [M−H]⁻.

Step 2. DIEA (20.2 μL, 118.5 μmol, 6.0 eq.) and HATU·HPF₆ (10.8 mg, 27.6 μmol, 1.4 eq.) were added to a solution of Fc-III—(OtBu)₂-L6Orn(Boc)-OH (40.1 mg, 19.7 μmol, 1.0 eq.) in DMF (0.2 mL) at rt. The reaction mixture was stirred at rt for 1 min and then N₃-PEG₁₁-NH₂ (13.5 mg, 23.7 μmol, 1.2 eq.) was added. After stirring at rt for 2 h, HATU·HPF₆ (3.1 mg, 7.9 μmol, 0.4 eq.) was added to the reaction mixture. After stirring at rt for 2.5 h, the reaction mixture was neutralized with TFA then purification by HPLC preparative (25 to 60% of ACN+0.1% FA in H2O+0.1% FA) afforded BocHN-Fc-III—(OtBu)₂-L6Orn-PEG₁₁-N₃ (19.4 mg, 7.9 μmol, UV purity 95%, 40% yield) as a white powder. UPLC-MS (method 4): Rt=3.21 min, m/z=1170 [M+2H]²⁺, 1168 [M-2H]²⁻.

Step 3. BocHN-Fc-III—(OtBu)₂-L6Orn-PEG₁₁-N₃ (19.4 mg, UV purity 95%, 7.9 μmol, 1.0 eq.) was dissolved in TFA (0.1 mL). After stirring at rt for 20 min, the reaction mixture was concentrated under vacuo, water (4 mL) and ACN (4 mL) were added and the mixture was freeze-dried to afford L6Orn-PEG₁₁-N₃ (17.7 mg, 5.4 mmol, UV purity 75%, 51% yield) as a white powder. UPLC-MS (method 4): Rt=2.15 min, m/z=1064 [M+2H]²⁺, 1062 [M-2H]²⁻.

The MS characterization of peptides for solid support immobilization is shown in the table below.

TABLE 21
Characterization of peptides for solid support immobilization

| | Cal- | Cal- | | | | |
|---|---|---|---|---|---|---|
| | culated | culated | Found | Retention | | UPLC- |
| | Exact | [M + | [M + | time | Purity | MS |
| Compound | Mass | 2H]²⁺ | 2H]²⁺ | (min) | (%) | method |
| Biotin- | 2356.86 | 1179.43 | 1179 | 2.293 | 98 | 2 |
| L6Orn | | | | | | |
| L6Orn(-H)- | 2527.07 | 1264.54 | 1264.2 | 2.267 | 100 | 2 |
| K(-biotin) | | | | | | |
| N₃-L6Orn | 2156.56 | 1079.28 | 1079.78 | 1.73 | 95 | 1 |
| L6Orn(-H)- | 2125.5 | 1063.75 | 1064 | 2.15 | 75 | 4 |
| N₃ | | | | | | |
| N₃-L6K | 2170.56 | 1086.28 | 1086 | 2.416 | 95 | 2 |

The Fc-binding ligands with azide or biotin were converted into the reactive conjugates by coupling of compound 006 or 009 to the amino group of side chain of peptides for solid support immobilization according to the same procedure as described in Example 6 or 5, respectively. The structures of the compounds prepared in Example 13 are shown in the table below.

TABLE 22
Structures of FITC/DOTA-containing reactive conjugates for solid support
immobilization.

| Compound | Structure |
|---|---|
| Biotin- L6Orn- FITC | 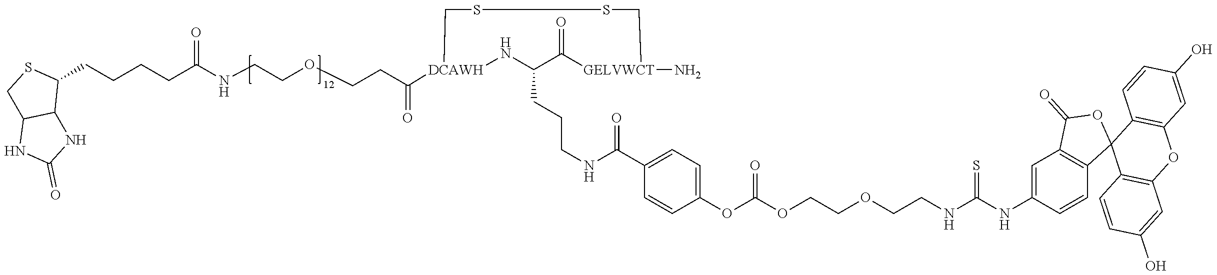 |
| L6Orn(-FITC)- K(-bio- tin) | 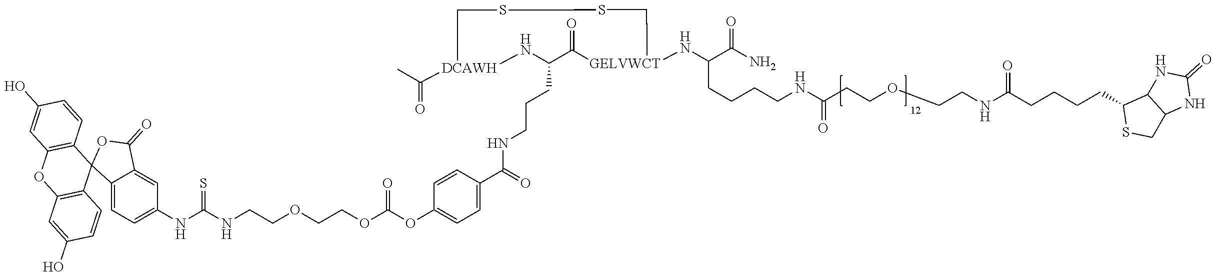 |
| N₃-L6Orn- FITC | 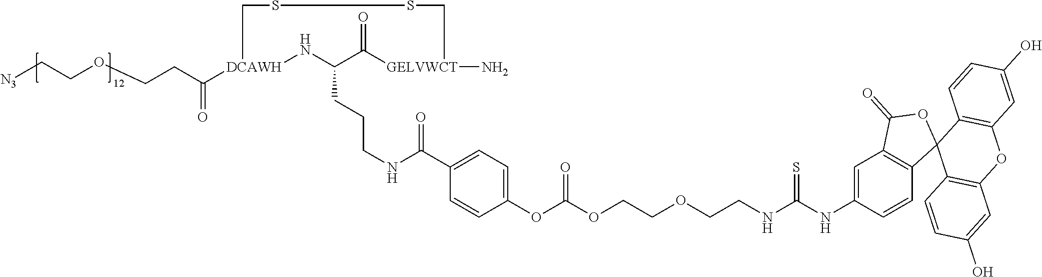 |
| L6Orn(-FITC)- N₃ | 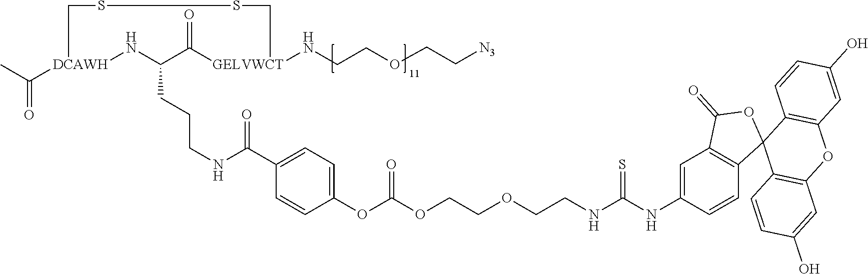 |
| L6Orn (-DOTA)-N₃ | 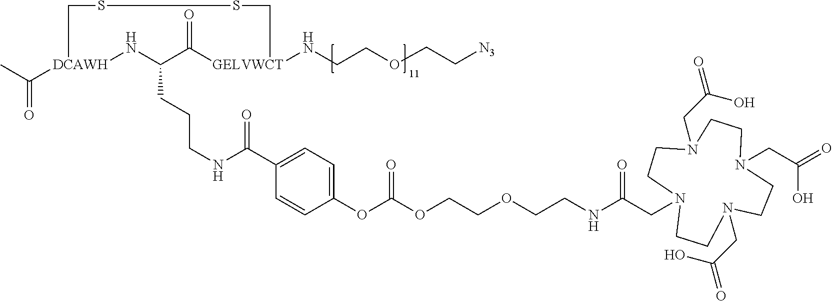 |
| N₃-L6K- FITC | 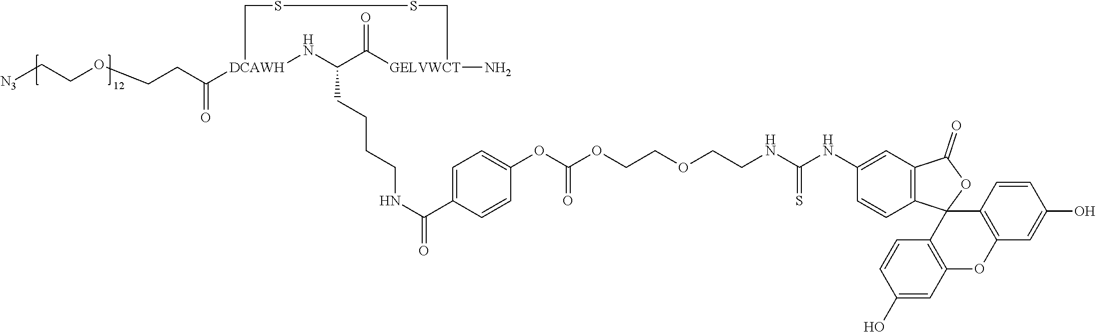 |

The MS characterization of FITC/DOTA-containing reactive conjugates for solid support immobilization is shown in the table below.

TABLE 23
Characterization of FITC/DOTA-containing reactive conjugates
for solid support immobilization

| | Cal- | Cal- | | | | |
|---|---|---|---|---|---|---|
| | culated | culated | Found | Retention | | UPLC- |
| | Exact | [M + | [M + | time | Purity | MS |
| Compound | Mass | 2H]²⁺ | 2H]²⁺ | (min) | (%) | method |
| Biotin- | 2997.86 | 1000.29 | 1000.81 | 2 | 98 | 1 |
| L6Orn- | | | | | | |
| FITC | | | | | | |
| L6Orn(- | 3168.07 | 1057.02 | 1057.54 | 1.97 | 97 | 1 |
| FITC)-K(- | | | | | | |
| biotin) | | | | | | |
| N₃-L6Orn- | 2797.56 | 1399.78 | 1399.1 | 2.907 | 91 | 2 |
| FITC | | | | | | |
| L6Orn(- | 2766.5 | 1384.25 | 1384.3 | 2.926 | 95 | 2 |
| FITC)-N₃ | | | | | | |
| L6Orn(- | 2762.5 | 1382.25 | 1382.8 | 2.458 | 100 | 2 |
| DOTA)-N₃ | | | | | | |
| N3-L6K- | 2811.56 | 1406.78 | 1407.15 | 2.15 | 91 | 1 |
| FITC | | | | | | |

The propensity of the reactive FITC/DOTA-containing reactive conjugates for solid support immobilization to react with an antibody was evaluated using trastuzumab as a model system. The trastuzumab-FITC/DOTA conjugates were first prepared in solution the same way as in Example 11.

The results of the HRMS analysis are shown in Table 24 below.

TABLE 24
Characterization of trastuzumab-FITC/DOTA
conjugates prepared in Example 13

| | | Labeled | | | |
|---|---|---|---|---|---|
| | | mAb | DoC | DoC | Selectivity |
| Compound | DoC | (%) | Fc | F(ab)₂ | Fc/F(ab)₂ |
| Biotin-L6Orn-FITC | 1.95 | 97.8 | 0.95 | 0.16 | 5.96 |
| L6Orn(-FITC)-K(- | 1.75 | 95.0 | 0.95 | 0.10 | 9.06 |
| biotin) | | | | | |
| N₃-L6Orn-FITC | 2.38 | 100.0 | 1.06 | 0.16 | 6.77 |
| L6Orn(-FITC)-N₃ | 2.62 | 99.6 | 1.12 | 0.31 | 3.55 |
| L6Orn(-DOTA)-N₃ | 1.84 | 97.8 | 1.74 | 0.17 | 10.06 |
| N₃-L6K-FITC | 1.27 | 77.7 | 0.58 | 0.27 | 2.13 |

To immobilize the biotinylated reactive conjugate on a solid support, NeutrAvidin Agarose Resin (Thermo Fisher) is packed into a column (Fisher Scientific) and washed with binding buffer (0.1 M phosphate buffer, 0.15 M sodium chloride, pH 7.2). A compound of the invention (2.1 nmol) is incubated with the washed NeutrAvidin agarose beads (40 μl beads: 7.5 μg peptide) for 30 min at room temperature.

The beads are washed 4 times with binding buffer and then, 50 mM NaHCO₃ pH 9.0 is added to increase the pH. Trastuzumab in PBS pH 7.0 (2.1 nmol) is added to the beads, the mixture is stirred for 2 h at room temperature, and then washed 3-4 times with binding buffer. Labeled Trastuzumab is eluted (100 μl, 0.1 M glycine, pH 2.5) into a collection tube containing neutralization buffer (1M phosphate buffer pH 8.5) at a 1:10 volumetric ratio. The elution step is repeated, and fractions are combined. The eluted labeled Trastuzumab is then buffer exchanged with PBS pH 7.0 using a 30 kDa MWCO Vivaspin® 500 centrifugal concentrators.

1. A compound of formula (1):
V—(Y—P)ₙ (1)
wherein:
V is a peptide comprising a vector that is capable of interacting with the fragment crystallizable (Fc) region of an antibody or fragment thereof, said antibody fragment optionally being incorporated into an Fc-fusion protein;
P is a group comprising one or more payloads P¹;
Y is a reactive moiety capable of reacting with the side chain of an amino acid, wherein Y is covalently attached to the side chain of an amino acid comprised in V; and
n is an integer from 1 to 3.

2. The compound of claim 1, wherein P is P¹, or P is represented by one of the following formulae (2a), (2b) and (2c):
P¹-L-* (2a)
(P¹-L)ₙ′-K—* (2b)
(P¹)ₙ′-K—* (2c)
wherein:
P¹ is a payload;
L is a linker, wherein said linker is optionally cleavable;
K is a branching group that is covalently bonded to group Y and to two or more linkers (L) in formula (2b), or two or more payloads (P¹) in formula (2c), so as to form a dendrimeric structure;
n′ is an integer of from 2 to 8; and
* indicates covalent attachment to the reactive moiety (Y).

3. The compound of claim 1, wherein P¹ is selected from the group consisting of:
(i) a moiety derived from:
a chromophore,
a labelling moiety which may include a radionuclide;
(ii) a moiety comprising a conjugation group selected from the group consisting of an optionally substituted conjugated diene, an optionally substituted tetrazine (TZ), an optionally substituted alkyne or azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, an optionally substituted halogenoacetamide, an optionally substituted maleimide, and an optionally substituted or protected thiol;
(iii) a moiety derived from a drug selected from the group consisting of:
an antineoplastic agent,
an immunomodulatory agent,
an anti-infectious disease agent, and
radioisotopes, metabolites, pharmaceutically acceptable salts, and/or prodrugs of any of the foregoing; and
(iv) a moiety comprising one or more solubilizing groups,
such that if more than one payload (P¹) is present, each P¹ is independently selected from the aforementioned moieties (i) to (iii) or (i) to (iv).

4. The compound of claim 1, wherein P¹ is a chelating agent that optionally comprises a chelated radionuclide.

5. The compound of claim 3, wherein the radionuclide is selected from the group consisting of ¹²⁴I, ¹³¹I, ⁸⁶Y, ⁹⁰Y, ¹⁷⁷Lu, ¹¹¹In, ¹⁸⁸Re, ⁵⁵Co, ⁶⁴Cu, ⁶⁷Cu, ⁶⁸Ga, ⁸⁹Zr, ²⁰³Pb, ²¹²Pb, ²¹²Bi, ²¹³Bi, ⁷²As, ²¹¹At, ²²⁵Ac, ²²³Ra, ⁹⁷Ru, ¹⁴⁹Tb, ¹⁵²Tb, ¹⁶¹Tb, ⁹⁹mTC, ²²⁶Th, ²²⁷Th, ²⁰¹Tl, ⁸⁹Sr, ^{44/43}Sc, ⁴⁷Sc, ¹⁵³Sm, ¹³³Xe, and Al¹⁸F.

6. The compound of claim 3, wherein P¹ is a moiety derived from exatecan, DM4, PNU-159682, amanitin, duocarmycin, auristatin, maytansine, tubulysin, calicheamicin, SN-38, taxol, tubulysin, daunomycin, vinblastine, doxorubicine, methotrexate, pyrrolobenzodiazepine, pyrrole-based kinesin spindle protein (KSP) inhibitors, indolino-benzodiazepine dimers, or radioisotopes and/or pharmaceutically acceptable salts thereof; and
if more than one payload (P¹) is present, each P¹ is independently selected from the aforementioned moieties (i) to (iii) or (i) to (iv).

7. The compound of claim 3, wherein at least one P¹ is a moiety derived from a compound comprising one or more polyethylene oxide groups, and
if more than one payload (P¹) is present, each P¹ is independently selected from the aforementioned moieties (i) to (iv).

8. The compound of claim 2, wherein the linker (L) is selected from the group consisting of:
(a1) an alkylene group having from 1 to 12 carbon atoms;
(b1) a polyalkylene oxide group with 2 or 3 carbon atoms having from 1 to 36 repeating units; and
(c1) a peptidic group comprising 2 to 12 amino acids, and;
if the compound of formula (1) comprises more than one linker (L), each L is independently selected from the aforementioned groups (a1) to (c1).

9. The compound of claim 2, wherein the branching group (K) is represented by the formula *—CH(R₁—**)(R₂—**) (3a), or the following formula (3b), 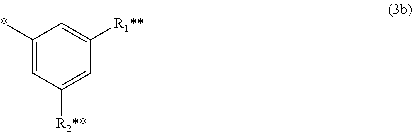
wherein,
R₁ and R₂ are each independently selected from the group consisting of —(CH₂)ₘ₁—** and —(CH₂)m₁R₃—**;
R₃ is selected from the group consisting of —NH—**, —(C═X)R₄—** and —NH(C═X)R₄—**;
R₄ is —(CH₂)ₘ₂—**, —(CH₂)ₘ₂S—** —CH(R₇—**)₂, —(CH₂)ₘ₂NH—**, or an aryl group of formula (3c): 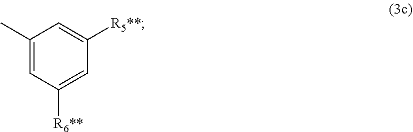
R₅ and R₆ are each independently selected from —(CH₂)ₘ₂—**, —(CH₂)ₘ₂S**—, —CH(CH₂S—**)₂, —(CH₂)ₘ₂NH—**, —CH(R₇—**)₂, —(CH₂)ₘ₂NH(C═X)R₈—**, and —CH₂(CH₂)ₘ₂H—**;
R₇ is —CH₂S—**, (CH₂)ₘ₂—**, or —(CH₂)ₘ₂R₉—**;
R₈ is —(CH₂)ₘ₃S—**;
R₉ is —NH(C═X)ₘ₃S—**;
each X is independently selected from O and S;
* indicates covalent attachment to the reactive moiety (Y), optionally via a linker (L2);
** indicates covalent attachment to linker (L) in formula (2b), or to payload (P¹) in formula (2c); and
m1, m2 and m3 are each independently selected from 0, 1, 2 and 3, with the proviso that if K is of formula (3a) m1 is not 0;

10. The compound of claim 2, wherein the branching group (K) is represented by one of the following formulae (3d) to (31): 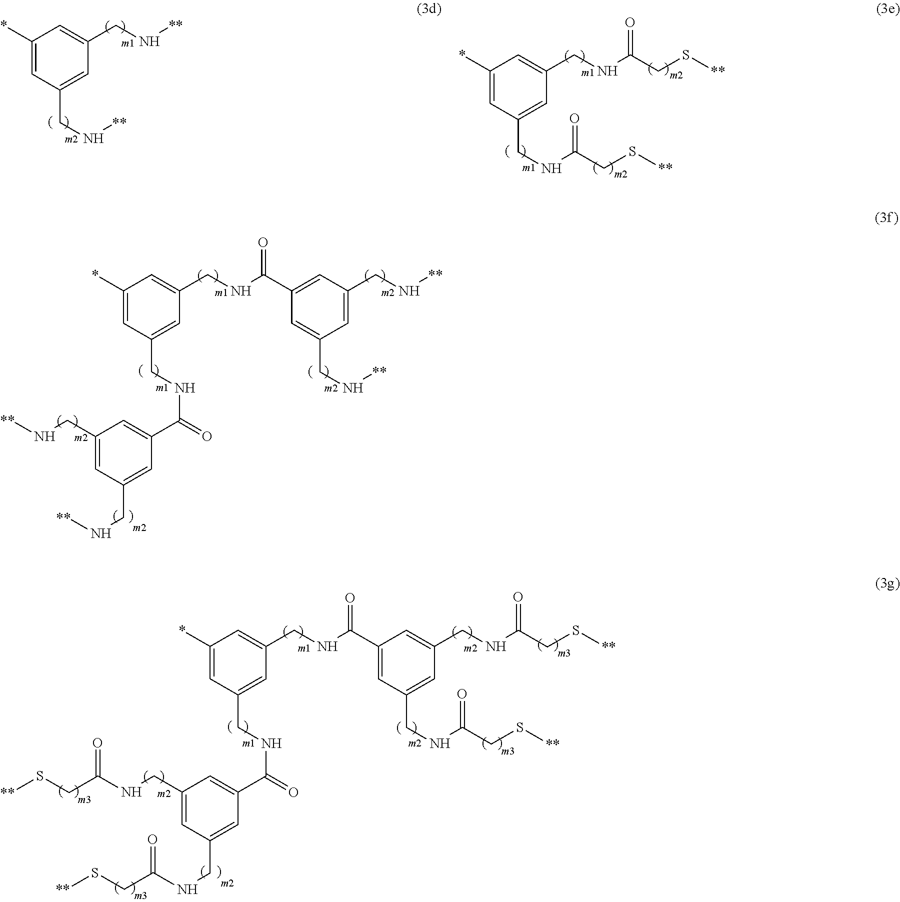 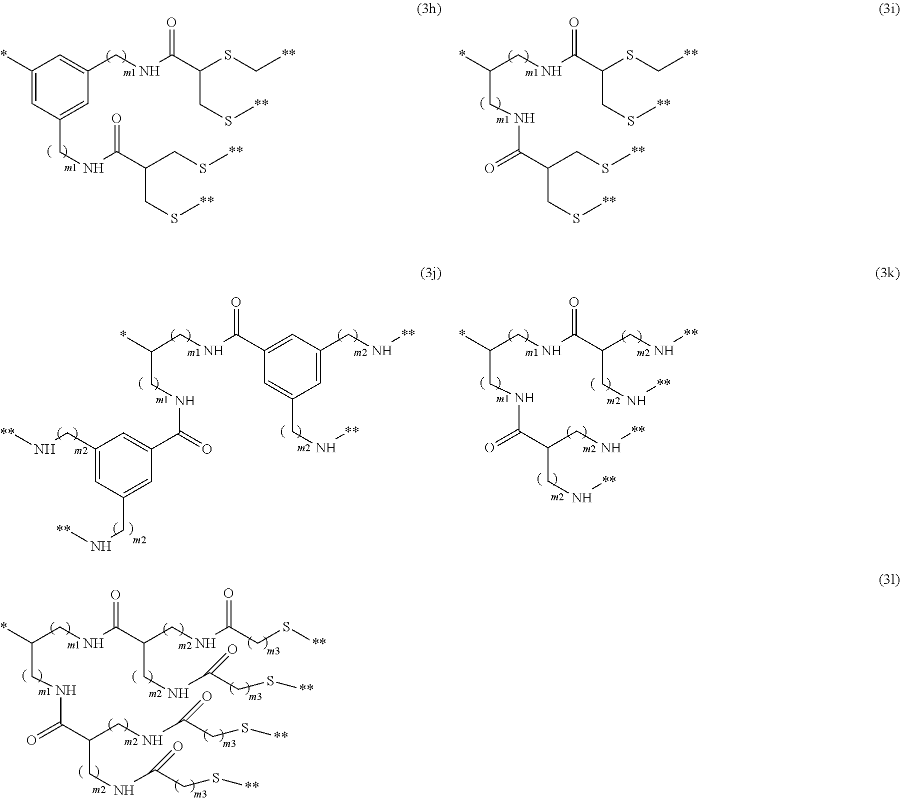
wherein:
m1 is 0, 1, 2 or 3;
m2 is 1, 2, 3 or 4;
m3 is 0, 1, 2 or 3;
* indicates covalent attachment to the reactive moiety (Y); and
** indicates covalent attachment to linker (L) or payload (P¹).

11. The compound of claim 2, wherein the branching group (K) is represented by the following formula (3m): 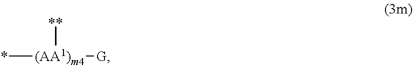
wherein:
m4 is an integer of 1 to 10;
each AA¹ is independently a moiety derived from a trifunctional amino acid, the side chain originating from the trifunctional amino acid is covalently attached to a linker (L) in formula (2b) or a payload (P¹) in formula (2c);
G is absent, or represents one selected from:
a hydrogen atom,
a group of formula —N(H)(R), wherein R is selected from a hydrogen atom, an alkyl group and a cycloalkyl group; and
a group of formula —N(H)(R)-(CH₂)ₙ₁₃—(C═O)N(H)(R), wherein each R is independently selected from the group consisting of a hydrogen atom, an alkyl group and a cycloalkyl group, and n13 is an integer of 1 to 6;
If G is absent, the resulting free valence forms a covalent bond to a linker (L) in formula (2b) or to a payload (P¹) in formula (2c);
* indicates covalent attachment to the reactive moiety (Y); and
** indicates covalent attachment to linker (L) or payload (P 1);
with the proviso that if m4 is 1, G is absent.

12. The compound of claim 1, wherein the reactive moiety (Y) is represented by one of the following formulae (4a) and (4b):
*′-F1-RC-F2-*** (4a)
*′-(F1-RC-F2)-M-*** (4b)
wherein:
RC is a reactive centre,
F1 is a single covalent bond, an atom, or a group of atoms;
F2 represents an atom, or a group of atoms;
M is a group capable of modulating the electron density and stability of F2;
*′ indicates covalent attachment to the group (P); and
*** indicates covalent attachment to the peptide (V).

13. The compound of claim 12, wherein the group (M) capable of modulating the electron density and stability of F2 is represented by the following formula (4c):
***‘--M’-B-E--*** (4c)
wherein:
M′ is a moiety derived from succinimide or an aryl group having 6, 10 or 14 ring members and 1, 2 or 3 condensed rings, respectively, or a heteroaryl group having 5 to 20 ring members, 1, 2 or 3 condensed rings and 1 to 4 heteroatoms independently selected from N, O and S, each group being optionally substituted with one or more substituents;
B is selected from:
a single covalent bond, O, S, or a group of atoms NR′, wherein R′ represents a hydrogen atom, —OH, an alkyl group, a cycloalkyl group, a C₂₋₆-alkenylene group, or a C₂₋₆-alkynylene group;
a group represented by the general formula (4d):
*—X¹—(CH₂CH₂O)ₙ₂—CH₂CH₂—** (4d)
wherein
X¹ is —C(═O)NH—, —C(═O)—, —NH— or —S—;
n2 is an integer of 1 to 24;
♦ indicates covalent attachment to M′, and
♦♦ indicates covalent attachment to E;
a peptidic group having 6 to 25 amino acids in the main chain;
and any combinations thereof,
E is C═O, C═S or C(═NR″), wherein R″ represents a hydrogen atom, OH, an alkyl group, a cycloalkyl group, S═O, or S(═O)₂;
***′ indicates covalent attachment to F2, and
*** indicates covalent attachment to the peptide (V).

14. The compound of claim 12, wherein the moiety (F1-RC-F2) is represented by one of the following formulae (4a′) to (41′) and/or the group (M) is represented by one of the following formulae (5a) to (5i′): 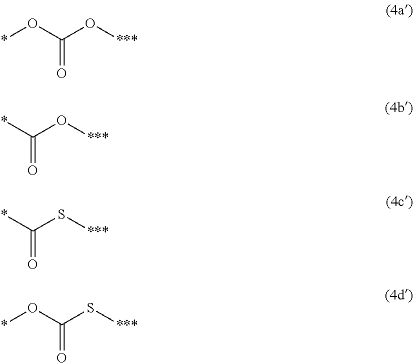 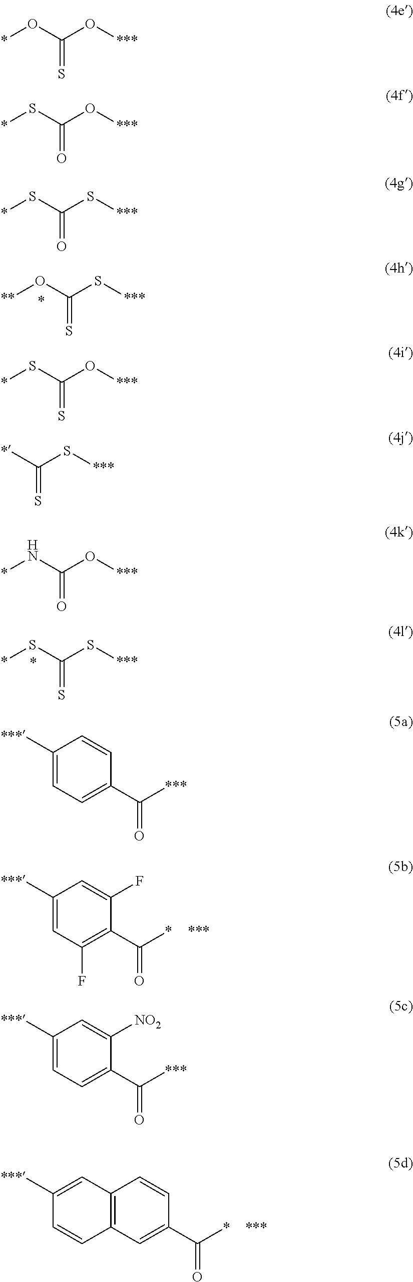 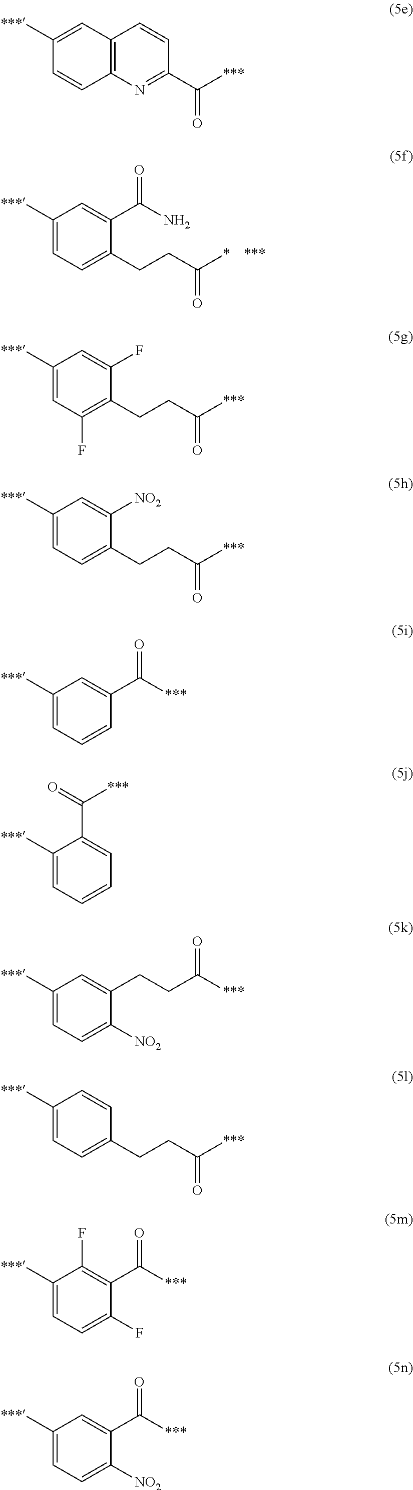 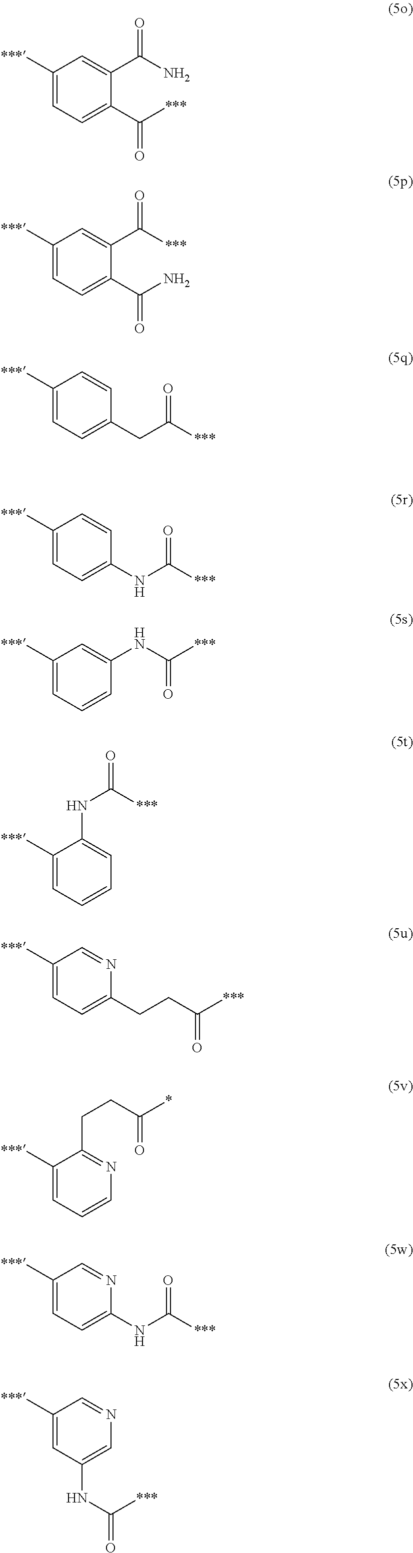 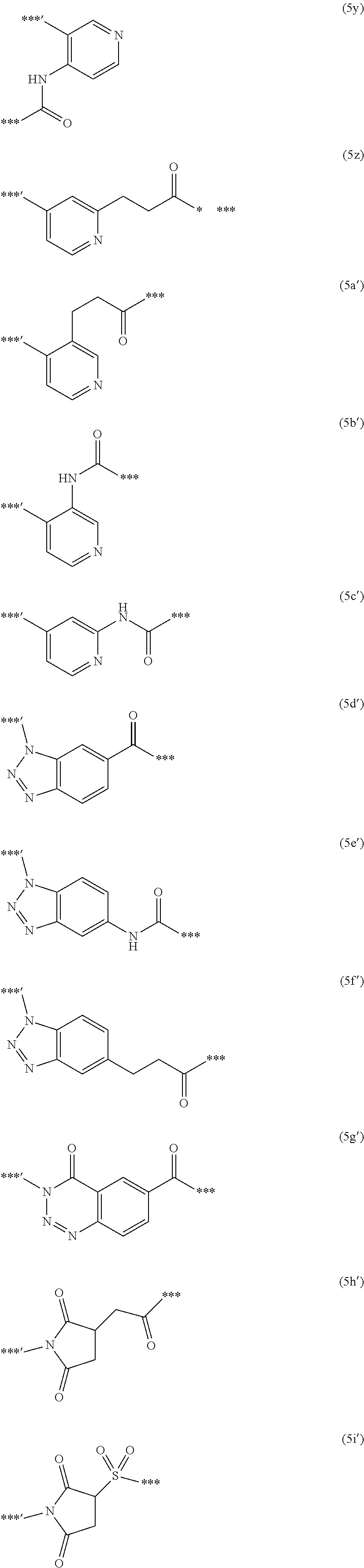
wherein:
*′ indicates covalent attachment to the group (P);
*** indicates covalent attachment to the peptide (V), or to the group (M) if M is present-; and
***′ indicates covalent attachment to F2 of the reactive moiety (Y).

15. The compound of claim 1, wherein the reactive moiety (Y) is represented by one of the following formulae (6a) to (6l′): 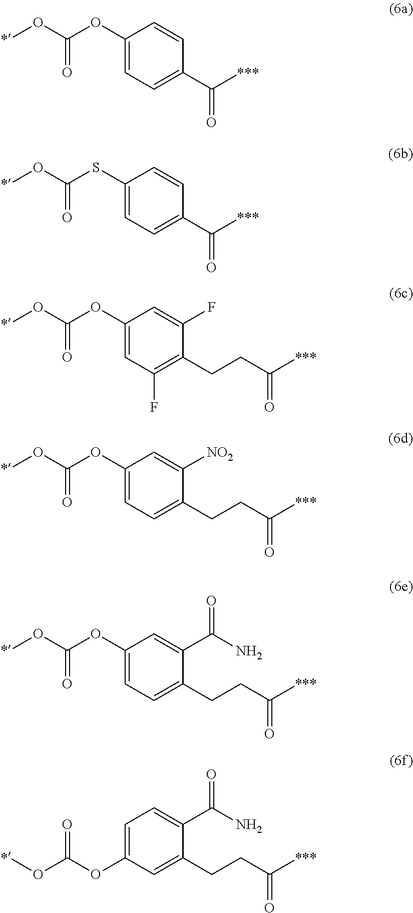 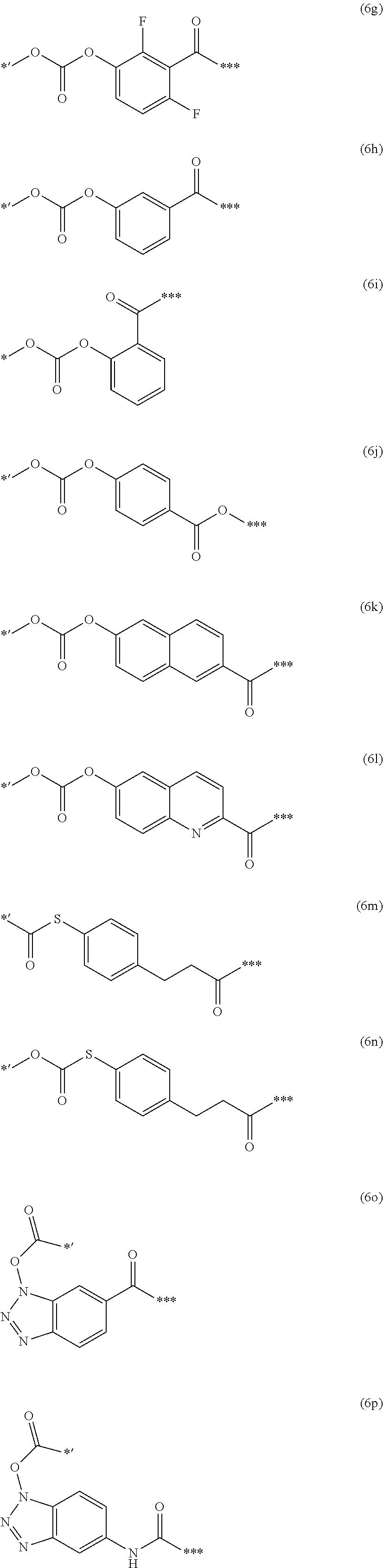 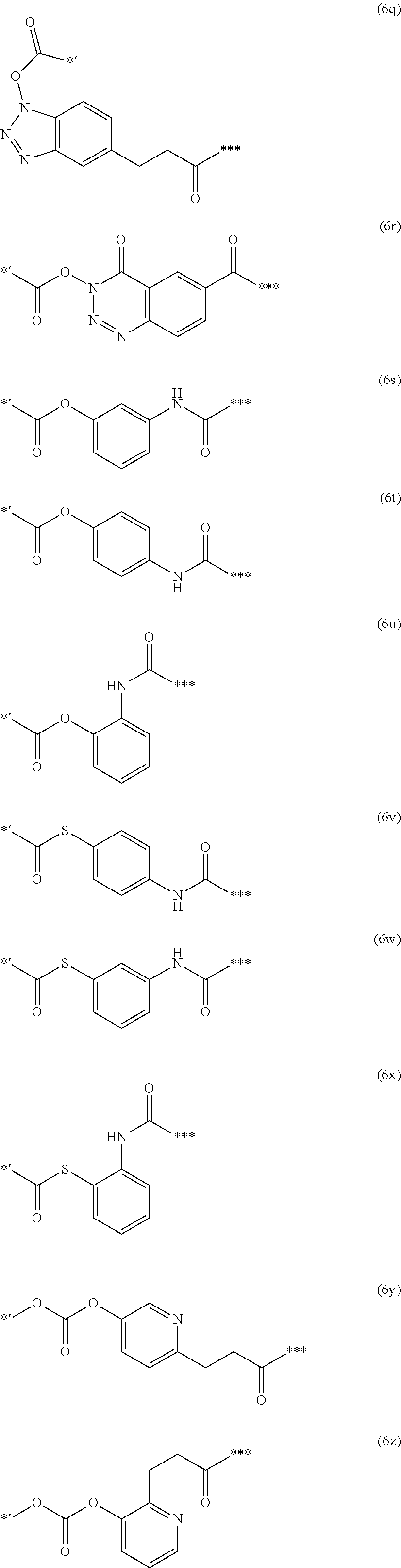 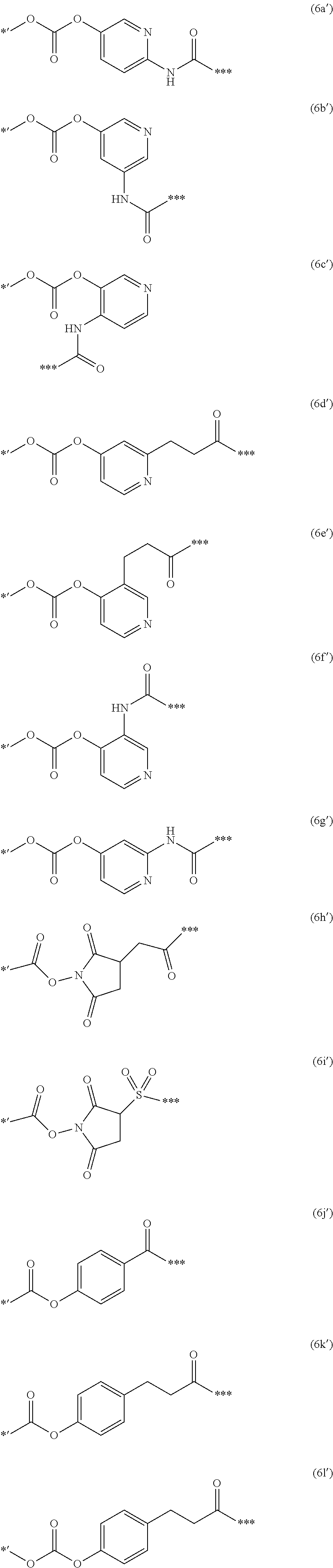
wherein:
*′ indicates covalent attachment to the group (P); and
*** indicates covalent attachment to the peptide (V).

16. The compound of claim 1, wherein the peptide (V) comprises a sequence of 11 to 17 amino acids which is represented by the following formula (7a): 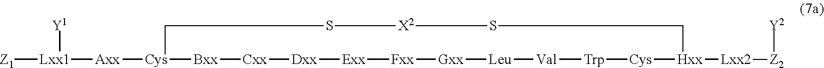
wherein:
Bxx represents an amino acid selected from an amino acid with an amino group-containing side chain, Ala, Tyr, homo-tyrosine (hTyr), and meta-tyrosine (mTyr);
Cxx, Dxx, Exx, Fxx, Gxx each independently represent an amino acid;
Axx represents an amino acid, a dicarboxylic acid, or a peptide moiety represented by the following formula (8a):
---Axx1-Axx2-Axx3--- (8a)
wherein, in formula (8a),
Axx1 represents a single covalent bond, or an amino acid such as Arg;
Axx2 represents an amino acid such as Gly or Cys, preferably Gly; and
Axx3 represents an amino acid such Asp or Asn;
Hxx represents an amino acid, or a peptide moiety represented by the following formula (8b):
---Hxx1-Hxx2-Hxx3--- (8b)
wherein, in formula (8b),
Hxx1 represents an amino acid such as Thr;
Hxx2 represents a single covalent bond or an amino acid such as Tyr or Cys; and
Hxx3 represents a single covalent bond, or an amino acid such as His; and the side chain of Axx2 may be covalently attached to the side chain of Hxx2 to form a ring;
if Axx2 is Cys and Hxx2 is Cys, the side chains of Axx2 and Hxx2 are linked together to form a group of formula —(S—X³—S)—, wherein X³ represents a single covalent bond or a divalent group comprising one or more atoms selected from carbon, nitrogen and oxygen such as a divalent maleimide group, a divalent acetone group or a divalent arylene group;
Lxx1 and Lxx2 each independently represent a single covalent bond or a trifunctional amino acid such as a diamino-carboxylic acid; with the proviso that at least one of Lxx1 and Lxx2 is a single covalent bond;
Z₁ represents a group selected from:
a group covalently attached to the N-terminus of Axx, if Lxx1 is a single covalent bond, which is selected from a hydrogen atom, a carbonyl-containing group, a C₂₋₃ polyalkylene oxide-containing group, and a group derived from a compound containing a conjugation group, an alkyne, an azide, a bromoacetamide, a maleimide and a thiol, wherein said conjugation group is optionally attached via a spacer (SI);
a group covalently attached to the N-terminus of Lxx1, if Lxx1 is a trifunctional amino acid and Y¹ is attached to the side chain of Lxx1, which is selected from a hydrogen atom, a carbonyl-containing group, and a C₂₋₃ polyalkylene oxide-containing group; and
a hydrogen atom or a C₂₋₃ polyalkylene oxide-containing group covalently attached to the side chain of Lxx1, if Lxx1 is a trifunctional amino acid and Y¹ is covalently attached to the N-terminus of Lxx1;
Z₂ represents a group selected from:
a group covalently attached to the C-terminus of Hxx, if Lxx2 is a single covalent bond, which is selected from —N(H)(R) wherein R represents a hydrogen atom, an alkyl or cycloalkyl group, a C₂₋₃ polyalkylene oxide-containing group, and a group derived from a compound containing a conjugation group, an alkyne, an azide, a bromoacetamide, a maleimide or a thiol, wherein said conjugation group is optionally attached via a spacer (S¹);
a group covalently attached to the C-terminus of Lxx2, if Lxx2 is a trifunctional amino acid and Y² is covalently attached to the side chain of Lxx2;
a hydrogen atom or a C₂₋₃ polyalkylene oxide-containing group, covalently attached to the side chain of Lxx2, if Lxx2 is trifunctional amino acid and Y² is covalently attached to the C-terminus of Lxx2;
Y¹ represents a moiety that is only present if Lxx1 is a trifunctional amino acid, wherein Y¹ is covalently attached to the side chain of Lxx1 if Z₁ is attached to the N-terminus of Lxx1, or to the N-terminus of Lxx1 if Z₁ is attached to the side chain of Lxx1;
wherein Y¹ is derived from a compound containing a conjugation group, and wherein said conjugation group is optionally attached via a spacer (S¹);
Y² represents a moiety that is only present if Lxx2 is a trifunctional amino acid, wherein Y² is covalently attached to the side chain of Lxx2 if Z₂ is attached to the C-terminus of Lxx2, or to the C-terminus of Lxx2 if Z₂ is attached to the side chain of Lxx2;
wherein Y² is derived from a compound containing a conjugation group, and wherein said conjugation group is optionally attached via a spacer (S1);
S¹ is a spacer represented by the following formula (8c):
α-X⁴—(CH₂CH₂O)ₙ₂—CH₂CH₂—X⁵-β (8c)
wherein:
X⁴ is NH, O or S;
X⁵ is NH or C═O;
n2 is an integer of 1 to 46; and
α indicates covalent attachment to Y¹ or Y² if X⁵ is covalently attached to the peptide (V), or covalent attachment to the peptide (V) if X⁵ is covalently attached to Y¹ or Y²; and
β indicates covalent attachment to the peptide (V) if X⁴ is covalently attached to
Y¹ or Y², or covalent attachment to Y¹ or Y² if X⁴ is covalently attached to the peptide (V);
X² represents a single covalent bond or a divalent group comprising one or more atoms selected from carbon, nitrogen and oxygen; and
wherein at least one of Axx, Bxx, Cxx, Dxx, Exx, Fxx, Gxx and Hxx represents an amino acid with an amino group-containing side chain, Tyr, hTyr, or mTyr, which is covalently attached to the reactive moiety (Y) via its side chain; with the proviso that the peptide (V) does not comprise more than three amino acids with an amino group-containing side chain.

17. The compound of claim 16, wherein at least one of Axx, Cxx, Dxx, Exx, Fxx, Gxx, Hxx, Lxx1 and Lxx2 is defined as follows:
Axx represents an amino acid selected from Ala, 2,3-diamino-propionic acid (Dap), Asp, Glu, 2-amino suberic acid, α-amino butyric acid, Asn and Gln, a dicarboxylic acid selected from succinic acid, glutaric acid and adipic acid; or a peptide moiety of formula (8a), wherein Axx1 is a single covalent bond, Axx2 is Cys, and Axx3 is Asp;
Cxx represents an amino acid selected from Trp, Phe, Tyr, phenylglycine (Phg), 3-benzothiopen-2-yl-L-alanine, 3-naphthalen-2-yl-L-alanine, 3-biphenyl-4-yl-L-alanine and 3-naphthalen-1-yl-L-alanine; preferably Trp;
Dxx represents an amino acid selected from His, Ala, 3-pyridin-2-yl-L-alanine, mTyr and Phe;
Exx represents an amino acid selected from Ala, 2-amino-butyric acid (Abu), Gly, Leu, Ile, Val, Met, cyclohexyl alanine (Cha), Phe, Thr, Cys, Tyr, and norleucine (Nle);
Fxx represents an amino acid selected from Ala, Gly, Asn, Ser, Abu, and Asp;
Gxx represents an amino acid selected from Ala, Glu, Asp, Gln, His, Arg, Ser, and Asn; preferably Asp or Glu; more preferably Glu;
Hxx represents an amino acid selected from Thr, Ser, Ala, Asn, Val, Abu, Ile, Met, Leu, Pro, Gln, and Cys; or a peptide moiety of formula (8b), wherein Hxx1 is Thr, Hxx2 is Cys, and Hxx3 is a single covalent bond;
Lxx1 and Lxx2 each independently represent an amino acid selected from Dap, Dab, Lys, Orn and hLys; and
if Dxx or Exx represents an amino acid with an amino group-containing side chain, Tyr, hTyr, or mTyr, which is covalently attached to the reactive moiety (Y) via its side chain, Gxx is Glu, Gln, His, Arg or Asn.

18. The compound of claim 16, wherein the peptide (V) is represented by the following formula (9a): 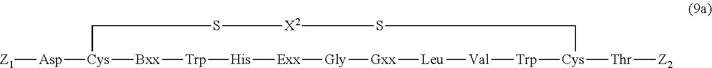
wherein,
Z₁, Z₂, Bxx, Exx, Gxx and X² are as defined in claim 16; and
at least one of Bxx, Exx and Gxx, represents an amino acid with an amino-group containing side chain, Tyr, hTyr, or mTyr; said amino acid being covalently bonded to the reactive moiety (Y) via its side chain; and
if Exx represents an amino acid with an amino group-containing side chain, Tyr, hTyr, or mTyr, which is covalently attached to the reactive moiety (Y) via its side chain, Gxx is Glu, Gln, His, Arg or Asn;

19. The compound of claim 14, wherein the peptide (V) is represented by one of the following formulae (10a) to (10v), (10b′) and (10g′): 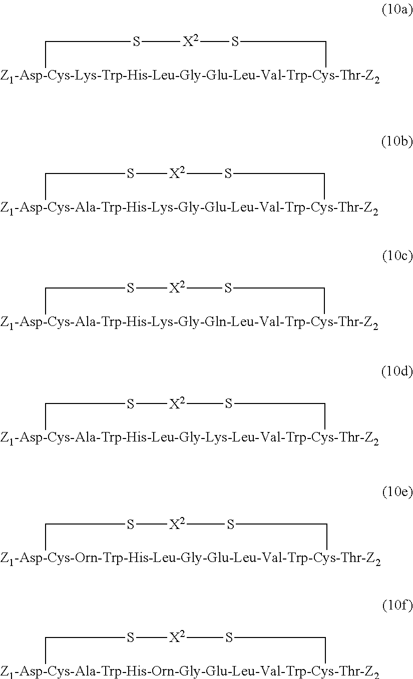 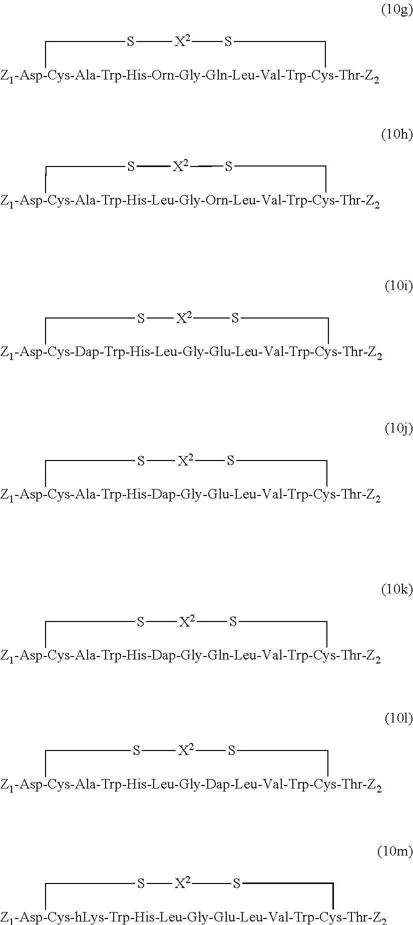 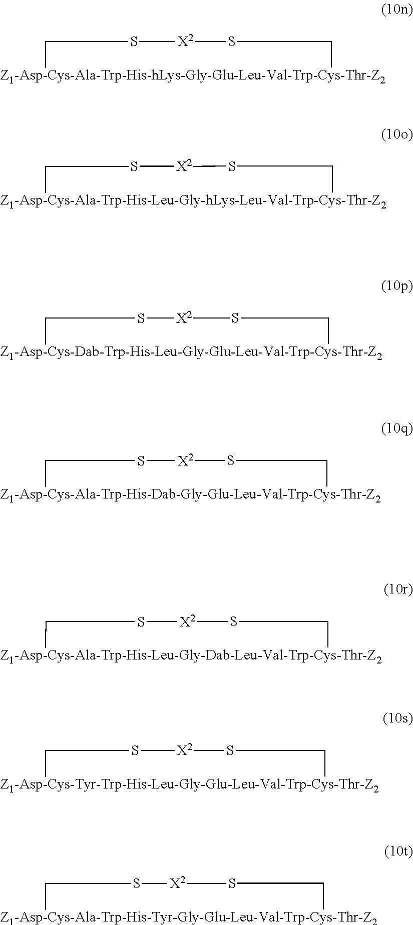 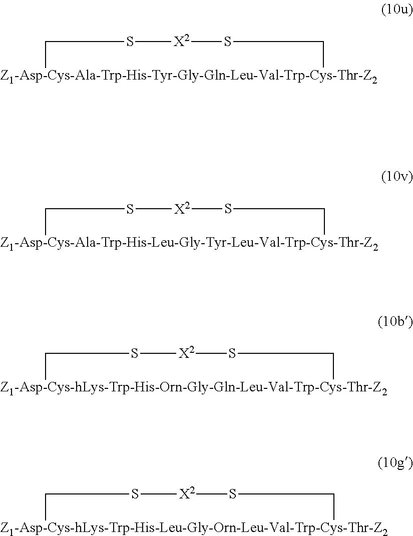
wherein,
Z₁, Z₂, and X² are as defined in claim 14; and
in the formulae (10a) to (10v), (10b′) and (10g′), the peptide (V) is covalently attached to the reactive moiety (Y) via the side chain of Tyr, Lys, hLys, Orn, Dap, or Dab comprised in V.

20. The compound of claim 16, wherein at least one of Z₁ and Z₂ is a C₂₋₃ polyalkylene oxide-containing group,
wherein,
Z₁ is represented by the following formula (13a):
X⁸—(OCH₂CH₂)ₙ₇—X⁹— (13a)
wherein
X⁸ represents an alkyl group having 1 to 6 carbon atoms such as an ethyl group, or an amino-containing group;
X⁹ represents —(C═X)—, X being selected from O and S; and
n7 is an integer of 4 to 100;
and/or
Z₂ is represented by the following formula (13b):
—X¹⁰—(CH₂CH₂O)ₙ₉—X¹¹ (13b)
wherein
n9 is an integer of 4 to 100;
X¹⁰ is a single covalent bond, NH, O or S;
X¹¹ represents an alkyl group having 1 to 6 carbon atoms such as a methyl group, a carbonyl-containing group such an acetyl group or a group of formula —(CH₂)ₙ₁₀—CO₂H, a thiocarbonyl-containing group, a group of formula —(CH₂)ₙ₁₀OR, a group of formula —(CH₂)ₙ₁₀—SO₃H, or an amino-containing group such as a group of formula —(CH₂)ₙ₁₀—(C═X)—N(R′)(R) or —(CH₂)ₙ₁₀—N(R′)(R), in which X is O or S, R and R′ are each independently selected from a hydrogen atom, an alkyl group or a cycloalkyl group, and n10 is an integer of 1 to 6.

21. The compound of claim 20, wherein Z₁ is a polyethylene oxide-containing group comprising from 10 to 200, and Z₂ is a group represented by —N(H)(R) wherein R represents a hydrogen atom, an alkyl group, or a cycloalkyl group.

22. The compound of claim 1, which is selected from the group consisting of:

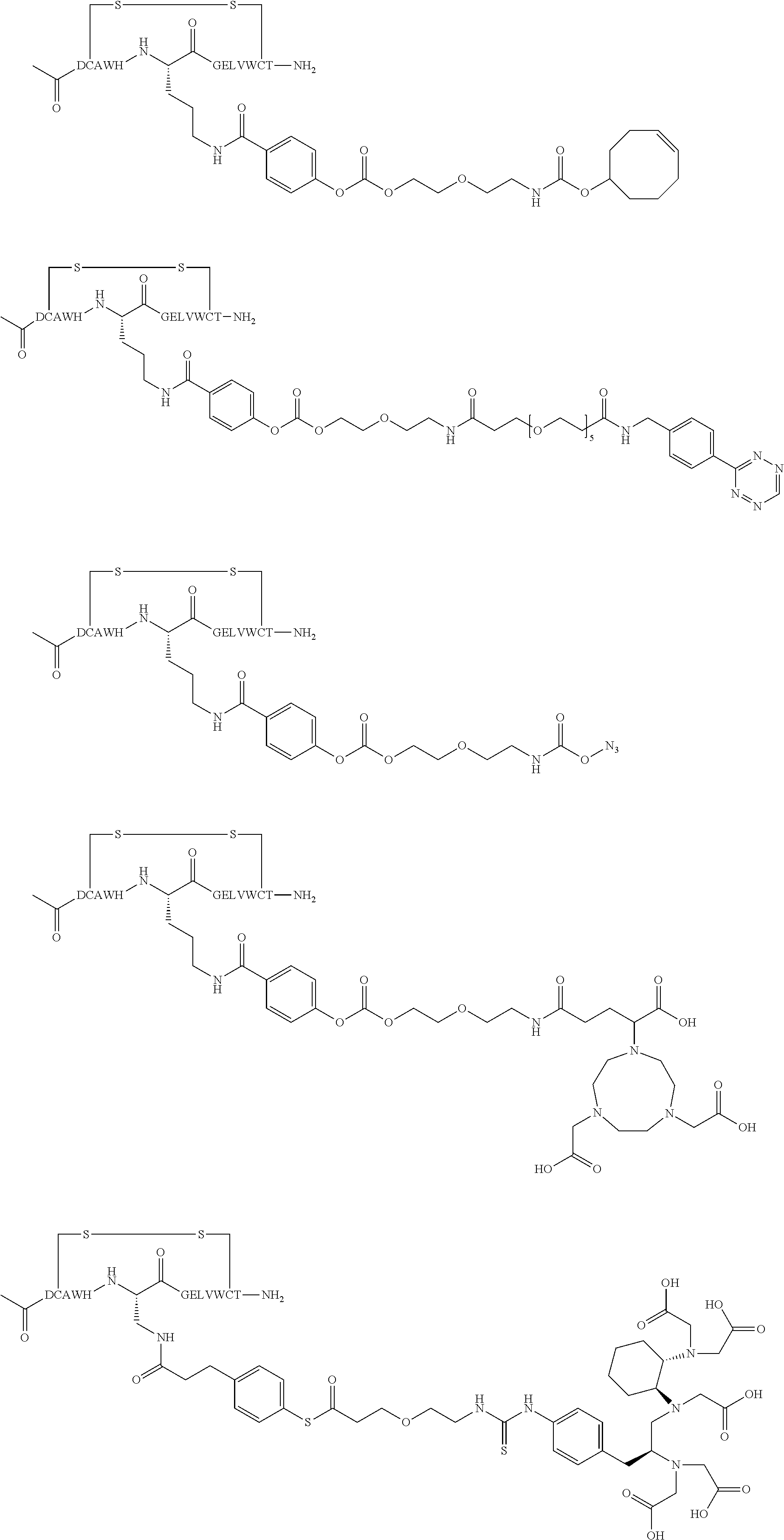

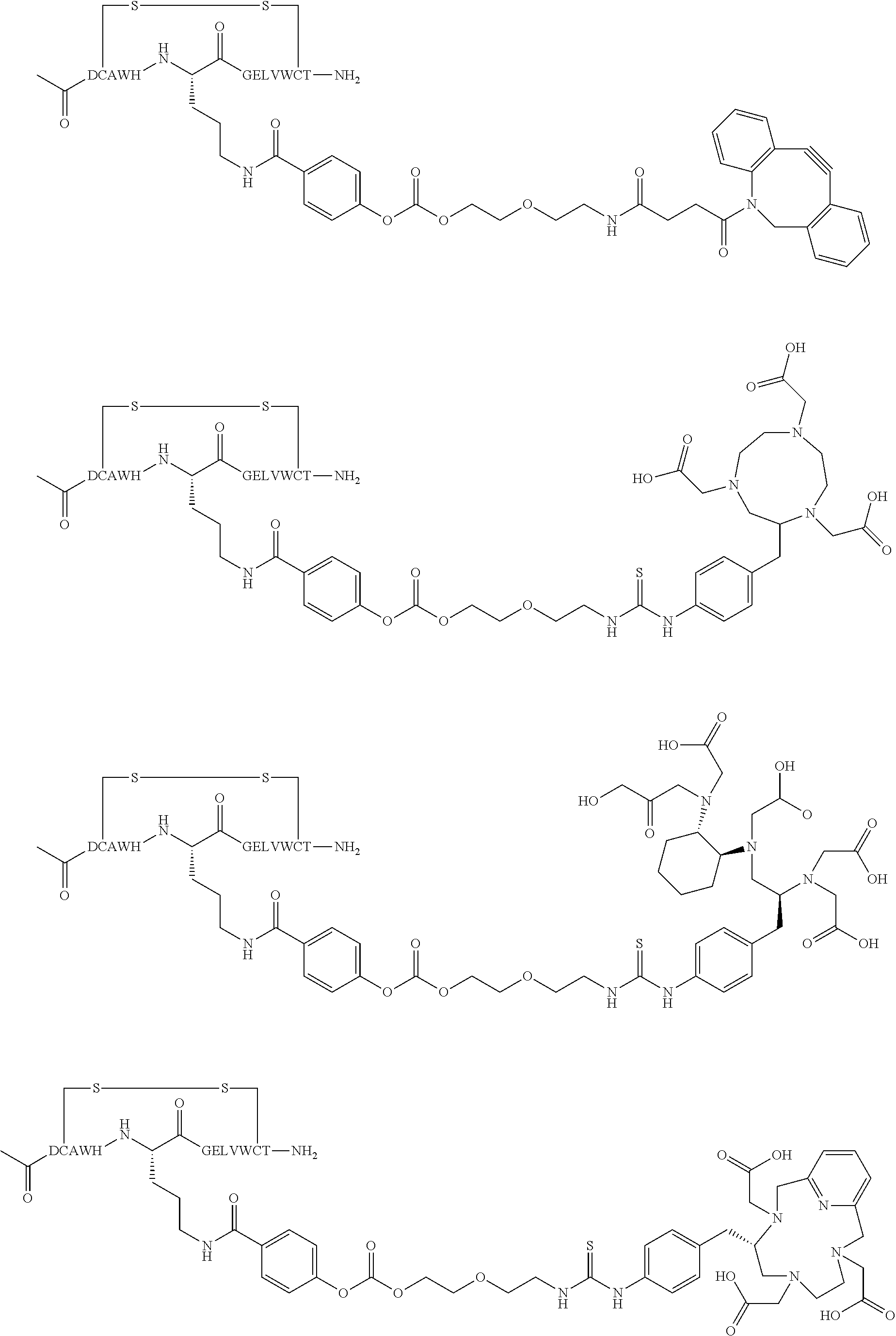

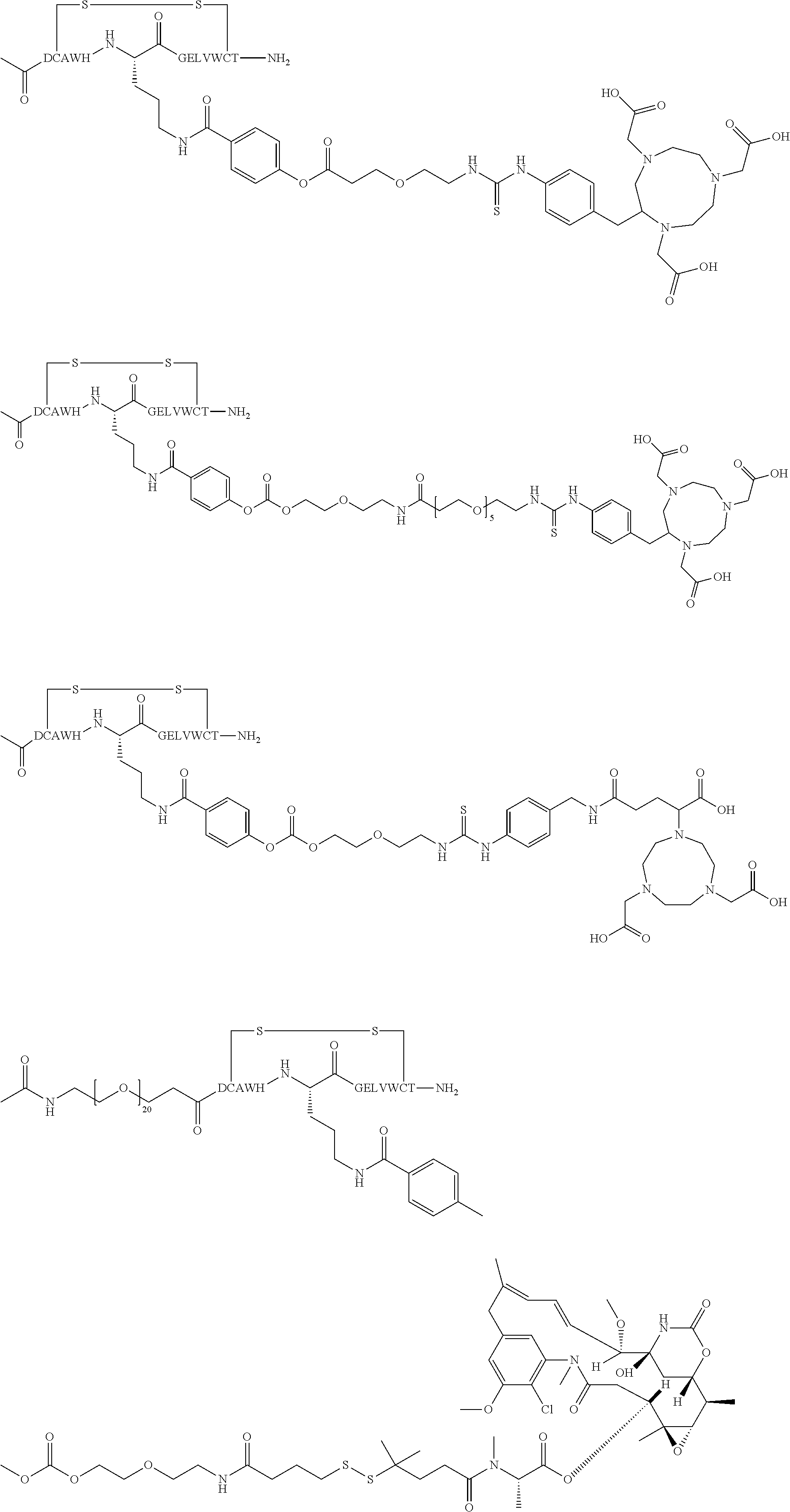

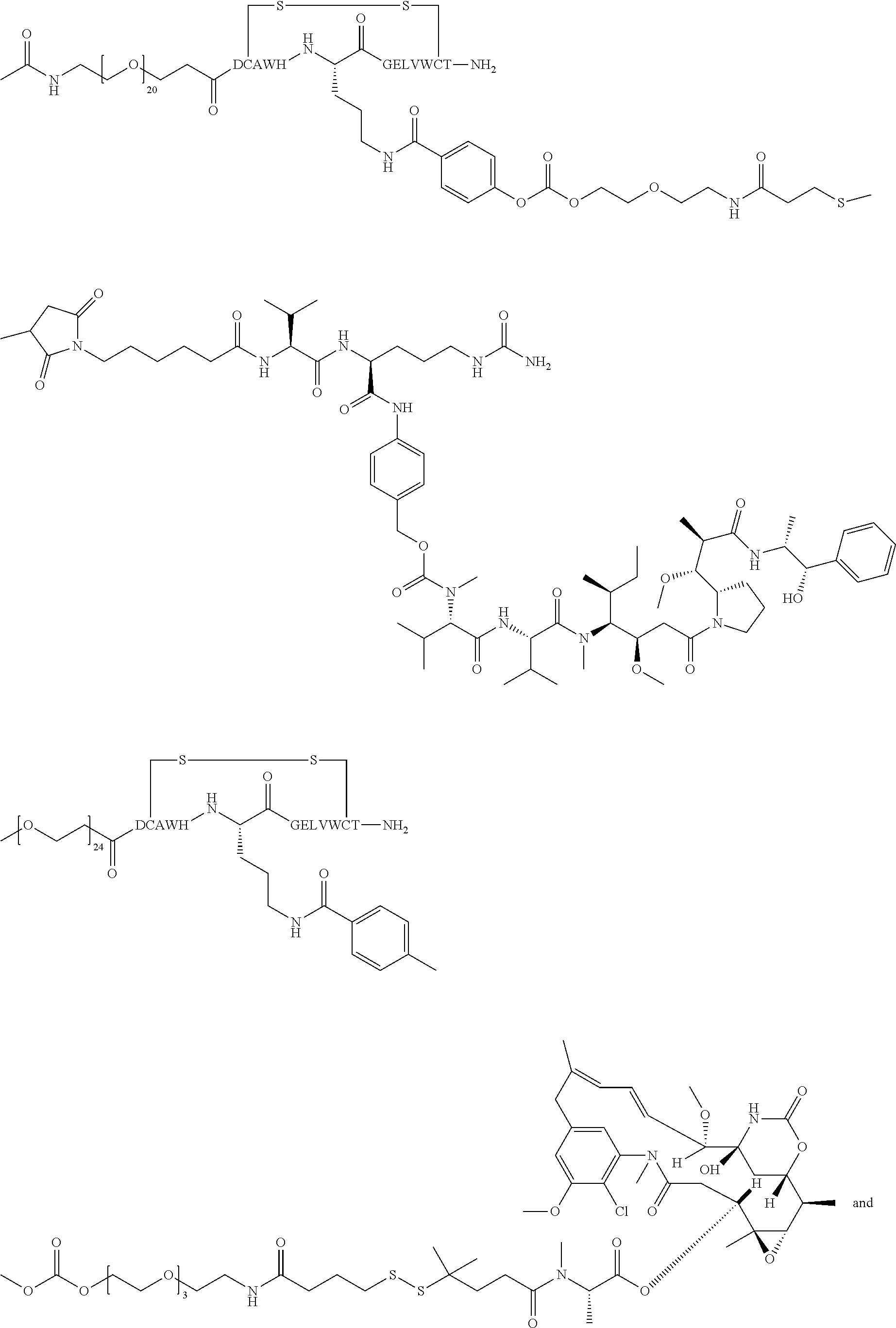

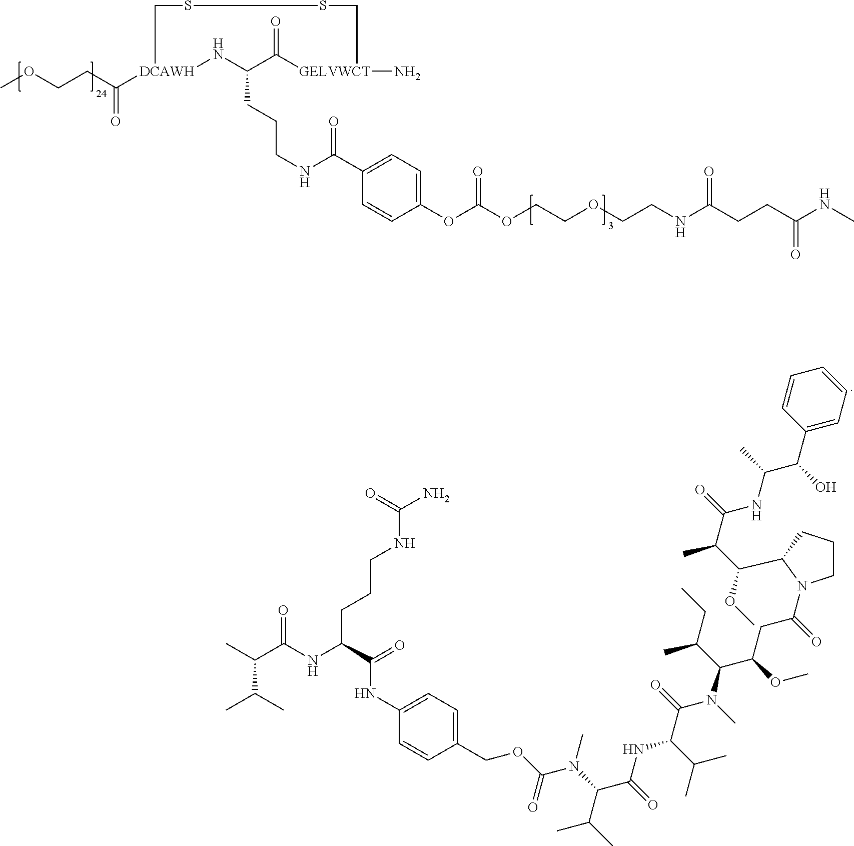

23. A kit for the regioselective modification of an antibody or fragment thereof, the antibody fragment being optionally incorporated into an Fc-fusion protein, the kit comprising the compound of claim 1 and a buffer.

24. The kit claim 23, wherein the compound is immobilized on a solid phase matrix, via a covalent linkage obtained by click reaction between an alkyne and azide, via covalent linkage obtained by reaction between a thiol and an acetamide, via covalent linkage obtained by reaction between a derivative of TCO and a derivative of TZ, or via covalent linkage obtained by reaction between a thiol and maleimide.

25. A method for the regioselective modification of an antibody or fragment thereof, the antibody fragment being optionally incorporated into an Fc-fusion protein, the method comprising reacting an antibody or fragment thereof with a compound according to claim 1.

26. The method of claim 25, wherein:
the antibody is a monoclonal antibody; or
the antibody fragment is incorporated into an Fc-fusion protein.

27. A modified antibody or modified antibody fragment obtainable by reacting an antibody or antibody fragment, the antibody fragment being optionally incorporated into an Fc-fusion protein, with a compound according to claim 1.

28. The modified antibody or modified antibody fragment of claim 27, which is represented by the following formula (11):
(P—W)ₚ-A (11)
wherein,
P is a group comprising one or more payloads (P¹), wherein P¹ is selected from the group consisting of:
(i) a moiety derived from:
a chromophore,
a labelling moiety which may include a radionuclide;
(ii) a moiety comprising a conjugation group selected from the group consisting of an optionally substituted conjugated diene, an optionally substituted tetrazine (TZ), an optionally substituted alkyne or azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, an optionally substituted halogenoacetamide, an optionally substituted maleimide, and an optionally substituted or protected thiol;
(iii) a moiety derived from a drug selected from the group consisting of:
an antineoplastic agent,
an immunomodulatory agent,
an anti-infectious disease agent, and
radioisotopes, metabolites, pharmaceutically acceptable salts, and/or prodrugs of any of the foregoing; and
(iv) a moiety comprising one or more solubilizing groups,
such that if more than one payload (P¹) is present, each P¹ is independently selected from the aforementioned moieties (i) to (iii) or (i) to (iv);
W is F1-RC′, wherein F1 is attached to P and RC′ is a moiety derived from a reactive center (RC) attached to A, F1 and RC F1 is a single covalent bond, an atom, or a group of atoms;
A is moiety derived from an antibody or an antibody fragment optionally incorporated into an Fc-fusion protein; and
p is an integer of 1 to 5.

29. A modified antibody or modified antibody fragment as defined in claim 27 for use in a method of diagnosing, monitoring, imaging or treating a disease and/or monitoring or imaging a treatment thereof, the method comprising administering the modified antibody or modified antibody fragment to a subject.

30. A method for diagnosing, monitoring, imaging or treating a disease comprising administering the modified antibody or modified antibody fragment according to claim 27 to a subject in need thereof.

31. The method according to claim 30, wherein the disease is a neurological disease, a cardiovascular disease, an auto-immune disease or a cancer.

32. The method according to claim 30, wherein the disease is selected from the group consisting of Alzheimer's Disease, Amyotrophic Lateral Sclerosis, Cerebral Arteriosclerosis, Encephalopathy, Huntington's Disease, Multiple Sclerosis, Parkinson's Disease, Progressive Multifocal Leukoencephalopathy, Systemic Lupus Erythematosus, systemic sclerosis, Angina including unstable angina, Aortic aneurysm, Atherosclerosis, Cardiac transplant, Cardiotoxicity diagnosis, Coronary artery bypass graft, Heart failure including atrial fibrillation terminated systolic heart failure, hypercholesterolaemia, Ischemia, Myocardial infarction, Thromboembolism, Thrombosis, Ankylosing spondylitis, Autoimmune cytopenias, Autoimmune myocarditis, Crohn's disease, Graft Versus Host disease, Granulomatosis with Polyangiitis, Idiopathic thrombocytopenic purpura, Juvenile arthritis, Juvenile diabetes (Type 1 diabetes), Lupus, Microscopic polyangiitis, Multiple sclerosis, Plaque psoriasis, Psoriasis, Psoriatic arthritis, Rheumatoid arthritis, Ulcerative colitis (UC), Uveitis, and Vasculitis.

33. The method according to claim 30, wherein the disease is cancer and involves cells selected from the group consisting of lymphoma cells, myeloma cells, renal cancer cells, breast cancer cells, prostate cancer cells, ovarian cancer cells, colorectal cancer cells, gastric cancer cells, squamous cancer cells, lung cancer cells, testicular cancer cells, pancreatic cancer cells, liver cancer cells, melanoma, head-and-neck cancer cells, and any cells growing and dividing at an unregulated and quickened pace to cause cancers; preferably selected from breast cancer cells, lung cancer cells, lymphoma cells, colorectal cancer cells, and head-and-neck cancer cells.
